SEQUENCE LISTING (12 sequences)

SEQ ID NO: 1 - protein, 2549 aa, Artificial Sequence
  Synthetic polypeptide
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 2 - protein, 1210 aa, Artificial Sequence [sequence omitted]

SEQ ID NO: 3 - protein, 1255 aa, Artificial Sequence [sequence omitted]

SEQ ID NO: 4 - protein, 2527 aa, Artificial Sequence
  Synthetic polypeptide
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 5 - protein, 189 aa, Artificial Sequence [sequence omitted]

SEQ ID NO: 6 - protein, 188 aa, Artificial Sequence [sequence omitted]

SEQ ID NO: 7 - protein, 2102 aa, Artificial Sequence
  Synthetic polypeptide
MAAAPARGGGGGGGGGGGCSGSGSSASRGFYFNTVLSLARSLAVQRPASLEKVQKLLCMCPVDFHGIFQLDERRRDAVIALGIFLIESDLQHKDCVVPYLLRLLKGLPKVYWVEESTARKGRGALPVAESFSFCLVTLLSDVAYRDPSLRDEILEVLLQVLHVLLGMCQALEIQDKEYLCKYAIPCLIGISRAFGRYSNMEESLLSKLFPKIPPHSLRVLEELEGVRRRSFNDFRSILPSNLLTVCQEGTLKRKTSSVSSISQVSPERGMPPPSSPGGSAFHYFEASCLPDGTALEPEYYFSTISSSFSVSPLFNGVTYKEFNIPLEMLRELLNLVKKIVEEAVLKSLDAIVASVMEANPSADLYYTSFSDPLYLTMFKMLRDTLYYMKDLPTSFVKEIHDFVLEQFNTSQGELQKILHDADRIHNELSPLKLRCQANAACVDLMVWAVKDEQGAENLCIKLSEKLQSKTSSKVIIAHLPLLICCLQGLGRLCERFPVVVHSVTPSLRDFLVIPSPVLVKLYKYHSQYHTVAGNDIKISVTNEHSESTLNVMSGKKSQPSMYEQLRDIAIDNICRCLKAGLTVDPVIVEAFLASLSNRLYISQESDKDAHLIPDHTIRALGHIAVALRDTPKVMEPILQILQQKFCQPPSPLDVLIIDQLGCLVITGNQYIYQEVWNLFQQISVKASSVVYSATKDYKDHGYRHCSLAVINALANIAANIQDEHLVDELLMNLLELFVQLGLEGKRASERASEKGPALKASSSAGNLGVLIPVIAVLTRRLPPIKEAKPRLQKLFRDFWLYSVLMGFAVEGSGLWPEEWYEGVCEIATKSPLLTFPSKEPLRSVLQYNSAMKNDTVTPAELSELRSTIINLLDPPPEVSALINKLDFAMSTYLLSVYRLEYMRVLRSTDPDRFQVMFCYFEDKAIQKDKSGMMQCVIAVADKVFDAFLNMMADKAKTKENEEELERHAQFLLVNFNHIHKRIRRVADKYLSGLVDKFPHLLWSGTVLKTMLDILQTLSLSLSADIHKDQPYYDIPDAPYRITVPDTYEARESIVKDFAARCGMILQEAMKWAPTVTKSHLQEYLNKHQNWVSGLSQHTGLAMATESILHFAGYNKQNTTLGATQLSERPACVKKDYSNFMASLNLRNRYAGEVYGMIRFSGTTGQMSDLNKMMVQDLHSALDRSHPQHYTQAMFKLTAMLISSKDCDPQLLHHLCWGPLRMFNEHGMETALACWEWLLAGKDGVEVPFMREMAGAWHMTVEQKFGLFSAEIKEADPLAASEASQPKPCPPEVTPHYIWIDFLVQRFEIAKYCSSDQVEIFSSLLQRSMSLNIGGAKGSMNRHVAAIGPRFKLLTLGLSLLHADVVPNATIRNVLREKIYSTAFDYFSCPPKFPTQGEKRLREDISIMIKFWTAMFSDKKYLTASQLVPPDNQDTRSNLDITVGSRQQATQGWINTYPLSSGMSTISKKSGMSKKTNRGSQLHKYYMKRRTLLLSLLATEIERLITWYNPLSAPELELDQAGENSVANWRSKYISLSEKQWKDNVNLAWSISPYLAVQLPARFKNTEAIGNEVTRLVRLDPGAVSDVPEAIKFLVTWHTIDADAPELSHVLCWAPTDPPTGLSYFSSMYPPHPLTAQYGVKVLRSFPPDAILFYIPQIVQALRYDKMGYVREYILWAASKSQLLAHQFIWNMKTNIYLDEEGHQKDPDIGDLLDQLVEEITGSLSGPAKDFYQREFDFFNKITNVSAIIKPYPKGDERKKACLSALSEVKVQPGCYLPSNPEAIVLDIDYKSGTPMQSAAKAPYLAKFKVKRCGVSELEKEGLRCRSDSEDECSTQEADGQKISWQAAIFKVGDDCRQDMLALQIIDLFKNIFQLVGLDLFVFPYRVVATAPGCGVIECIPDCTSRDQLGRQTDFGMYDYFTRQYGDESTLAFQQARYNFIRSMAAYSLLLFLLQIKDRHNGNIMLDKKGHIIHIDFGFMFESSPGGNLGWEPDIKLTDEMVMIMGGKMEATPFKWFMEMCVRGYLAVRPYMDAVVSLVTLMLDTGLPCFRGQTIKLLKHRFSPNMTEREAANFIMKVIQSCFLSNRSRTYDMIQYYQNDIPY

SEQ ID NO: 8 - protein, 562 aa, Artificial Sequence
  Synthetic polypeptide
MASASSGPSSSVGFSSFDPAVPSCTLSSAASGIKRPMASEVLEARQDSYISLVPYASGMPIKKIGHRSVDSSGETTYKKTTSSALKGAIQLGITHTVGSLSTKPERDVLMQDFYVVESIFFPSEGSNLTPAHHYNDFRFKTYAPVAFRYFRELFGIRPDDYLYSLCSEPLIELCSSGASGSLFYVSSDDEFIIKTVQHKEAEFLQKLLPGYYMNLNQNPRTLLPKFYGLYCVQAGGKNIRIVVMNNLLPRSVKMHIKYDLKGSTYKRRASQKEREKPLPTFKDLDFLQDIPDGLFLDADMYNALCKTLQRDCLVLQSFKIMDYSLLMSIHNIDHAQREPLSSETQYSVDTRRPAPQKALYSTAMESIQGEARRGGTMETDDHMGGIPARNSKGERLLLYIGIIDILQSYRFVKKLEHSWKALVHDGDTVSVHRPGFYAERFQRFMCNTVFKKIPLKPSPSKKFRSGSSFSRRAGSSGNSCITYQPSVSGEHKAQVTTKAEVEPGVHLGRPDVLPQTPPLEEISEGSPIPDPSFSPLVGETLQMLTTSTTLEKLEVAESEFTH

SEQ ID NO: 9 - protein, 594 aa, Artificial Sequence
  Synthetic polypeptide
MGKKSRVKTQKSGTGATATVSPKEILNLTSELLQKCSSPAPGPGKEWEEYVQIRTLVEKIRKKQKGLSVTFDGKREDYFPDLMKWASENGASVEGFEMVNFKEEGFGLRATRDIKAEELFLWVPRKLLMTVESAKNSVLGPLYSQDRILQAMGNIALAFHLLCERASPNSFWQPYIQTLPSEYDTPLYFEEDEVRYLQSTQAIHDVFSQYKNTARQYAYFYKVIQTHPHANKLPLKDSFTYEDYRWAVSSVMTRQNQIPTEDGSRVTLALIPLWDMCNHTNGLITTGYNLEDDRCECVALQDFRAGEQIYIFYGTRSNAEFVIHSGFFFDNNSHDRVKIKLGVSKSDRLYAMKAEVLARAGIPTSSVFALHFTEPPISAQLLAFLRVFCMTEEELKEHLLGDSAIDRIFTLGNSEFPVSWDNEVKLWTFLEDRASLLLKTYKTTIEEDKSVLKNHDLSVRAKMAIKLRLGEKEILEKAVKSAAVNREYYRQQMEEKAPLPKYEESNLGLLESSVGDSRLPLVLRNLEEEAGVQDALNIREAISKAKATENGLVNGENSIPNGTRSENESLNQESKRAVEDAKGSSSDSTAGVKE

SEQ ID NO: 10 - protein, 3859 aa, Artificial Sequence
  Synthetic polypeptide
MAFVATQGATVVDQTTLMKKYLQFVAALTDVNTPDETKLKMMQEVSENFENVTSSPQYSTFLEHIIPRFLTFLQDGEVQFLQEKPAQQLRKLVLEIIHRIPTNEHLRPHTKNVLSVMFRFLETENEENVLICLRIIIELHKQFRPPITQEIHHFLDFVKQIYKELPKVVNRYFENPQVIPENTVPPPEMVGMITTIAVKVNPEREDSETRTHSIIPRGSLSLKVLAELPIIVVLMYQLYKLNIHNVVAEFVPLIMNTIAIQVSAQARQHKLYNKELYADFIAAQIKTLSFLAYIIRIYQELVTKYSQQMVKGMLQLLSNCPAETAHLRKELLIAAKHILTTELRNQFIPCMDKLFDESILIGSGYTARETLRPLAYSTLADLVHHVRQHLPLSDLSLAVQLFAKNIDDESLPSSIQTMSCKLLLNLVDCIRSKSEQESGNGRDVLMRMLEVFVLKFHTIARYQLSAIFKKCKPQSELGAVEAALPGVPTAPAAPGPAPSPAPVPAPPPPPPPPPPATPVTPAPVPPFEKQGEKDKEDKQTFQVTDCRSLVKTLVCGVKTITWGITSCKAPGEAQFIPNKQLQPKETQIYIKLVKYAMQALDIYQVQIAGNGQTYIRVANCQTVRMKEEKEVLEHFAGVFTMMNPLTFKEIFQTTVPYMVERISKNYALQIVANSFLANPTTSALFATILVEYLLDRLPEMGSNVELSNLYLKLFKLVFGSVSLFAAENEQMLKPHLHKIVNSSMELAQTAKEPYNYFLLLRALFRSIGGGSHDLLYQEFLPLLPNLLQGLNMLQSGLHKQHMKDLFVELCLTVPVRLSSLLPYLPMLMDPLVSALNGSQTLVSQGLRTLELCVDNLQPDFLYDHIQPVRAELMQALWRTLRNPADSISHVAYRVLGKFGGSNRKMLKESQKLHYVVTEVQGPSITVEFSDCKASLQLPMEKAIETALDCLKSANTEPYYRRQAWEVIKCFLVAMMSLEDNKHALYQLLAHPNFTEKTIPNVIISHRYKAQDTPARKTFEQALTGAFMSAVIKDLRPSALPFVASLIRHYTMVAVAQQCGPFLLPCYQVGSQPSTAMFHSEENGSKGMDPLVLIDAIAICMAYEEKELCKIGEVALAVIFDVASIILGSKERACQLPLFSYIVERLCACCYEQAWYAKLGGVVSIKFLMERLPLTWVLQNQQTFLKALLFVMMDLTGEVSNGAVAMAKTTLEQLLMRCATPLKDEERAEEIVAAQEKSFHHVTHDLVREVTSPNSTVRKQAMHSLQVLAQVTGKSVTVIMEPHKEVLQDMVPPKKHLLRHQPANAQIGLMEGNTFCTTLQPRLFTMDLNVVEHKVFYTELLNLCEAEDSALTKLPCYKSLPSLVPLRIAALNALAACNYLPQSREKIIAALFKALNSTNSELQEAGEACMRKFLEGATIEVDQIHTHMRPLLMMLGDYRSLTLNVVNRLTSVTRLFPNSFNDKFCDQMMQHLRKWMEVVVITHKGGQRSDGNESISECGRCPLSPFCQFEEMKICSAIINLFHLIPAAPQTLVKPLLEVVMKTERAMLIEAGSPFREPLIKFLTRHPSQTVELFMMEATLNDPQWSRMFMSFLKHKDARPLRDVLAANPNRFITLLLPGGAQTAVRPGSPSTSTMRLDLQFQAIKIISIIVKNDDSWLASQHSLVSQLRRVWVSENFQERHRKENMAATNWKEPKLLAYCLLNYCKRNYGDIELLFQLLRAFTGRFLCNMTFLKEYMEEEIPKNYSIAQKRALFFRFVDFNDPNFGDELKAKVLQHILNPAFLYSFEKGEGEQLLGPPNPEGDNPESITSVFITKVLDPEKQADMLDSLRIYLLQYATLLVEHAPHHIHDNNKNRNSKLRRLMTFAWPCLLSKACVDPACKYSGHLLLAHIIAKFAIHKKIVLQVFHSLLKAHAMEARAIVRQAMAILTPAVPARMEDGHQMLTHWTRKIIVEEGHTVPQLVHILHLIVQHFKVYYPVRHHLVQHMVSAMQRLGFTPSVTIEQRRLAVDLSEVVIKWELQRIKDQQPDSDMDPNSSGEGVNSVSSSIKRGLSVDSAQEVKRFRTATGAISAVFGRSQSLPGADSLLAKPIDKQHTDTVVNFLIRVACQVNDNTNTAGSPGEVLSRRCVNLLKTALRPDMWPKSELKLQWFDKLLMTVEQPNQVNYGNICTGLEVLSFLLTVLQSPAILSSFKPLQRGIAACMTCGNTKVLRAVHSLLSRLMSIFPTEPSTSSVASKYEELECLYAAVGKVIYEGLTNYEKATNANPSQLFGTLMILKSACSNNPSYIDRLISVFMRSLQKMVREHLNPQAASGSTEATSGTSELVMLSLELVKTRLAVMSMEMRKNFIQAILTSLIEKSPDAKILRAVVKIVEEWVKNNSPMAANQTPTLREKSILLVKMMTYIEKRFPEDLELNAQFLDLVNYVYRDETLSGSELTAKLEPAFLSGLRCAQPLIRAKFFEVFDNSMKRRVYERLLYVTCSQNWEAMGNHFWIKQCIELLLAVCEKSTPIGTSCQGAMLPSITNVINLADSHDRAAFAMVTHVKQEPRERENSESKEEDVEIDIELAPGDQTSTPKTKELSEKDIGNQLHMLTNRHDKFLDTLREVKTGALLSAFVQLCHISTTLAEKTWVQLFPRLWKILSDRQQHALAGEISPFLCSGSHQVQRDCQPSALNCFVEAMSQCVPPIPIRPCVLKYLGKTHNLWFRSTLMLEHQAFEKGLSLQIKPKQTTEFYEQESITPPQQEILDSLAELYSLLQEEDMWAGLWQKRCKYSETATAIAYEQHGFFEQAQESYEKAMDKAKKEHERSNASPAIFPEYQLWEDHWIRCSKELNQWEALTEYGQSKGHINPYLVLECAWRVSNWTAMKEALVQVEVSCPKEMAWKVNMYRGYLAICHPEEQQLSFIERLVEMASSLAIREWRRLPHVVSHVHTPLLQAAQQIIELQEAAQINAGLQPTNLGRNNSLHDMKTVVKTWRNRLPIVSDDLSHWSSIFMWRQHHYQGKPTWSGMHSSSIVTAYENSSQHDPSSNNAMLGVHASASAIIQYGKIARKQGLVNVALDILSRIHTIPTVPIVDCFQKIRQQVKCYLQLAGVMGKNECMQGLEVIESTNLKYFTKEMTAEFYALKGMFLAQINKSEEANKAFSAAVQMHDVLVKAWAMWGDYLENIFVKERQLHLGVSAITCYLHACRHQNESKSRKYLAKVLWLLSFDDDKNTLADAVDKYCIGVPPIQWLAWIPQLLTCLVGSEGKLLLNLISQVGRVYPQAVYFPIRTLYLTLKIEQRERYKSDPGPIRATAPMWRCSRIMHMQRELHPTLLSSLEGIVDQMVWFRENWHEEVLRQLQQGLAKCYSVAFEKSGAVSDAKITPHTLNFVKKLVSTFGVGLENVSNVSTMFSSAASESLARRAQATAQDPVFQKLKGQFTTDFDFSVPGSMKLHNLISKLKKWIKILEAKTKQLPKFFLIEEKCRFLSNFSAQTAEVEIPGEFLMPKPTHYYIKIARFMPRVEIVQKHNTAARRLYIRGHNGKIYPYLVMNDACLTESRREERVLQLLRLLNPCLEKRKETTKRHLFFTVPRVVAVSPQMRLVEDNPSSLSLVEIYKQRCAKKGIEHDNPISRYYDRLATVQARGTQASHQVLRDILKEVQSNMVPRSMLKEWALHTFPNATDYWTFRKMFTIQLALIGFAEFVLHLNRLNPEMLQIAQDTGKLNVAYFRFDINDATGDLDANRPVPFRLTPNISEFLTTIGVSGPLTASMIAVARCFAQPNFKVDGILKTVLRDEIIAWHKKTQEDTSSPLSAAGQPENMDSQQLVSLVQKAVTAIMTRLHNLAQFEGGESKVNTLVAAANSLDNLCRMDPAWHPWL

SEQ ID NO: 11 - protein, 1239 aa, Artificial Sequence
  Synthetic polypeptide
MANDSPAKSLVDIDLSSLRDPAGIFELVEVVGNGTYGQVYKGRHVKTGQLAAIKVMDVTEDEEEEIKLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGAPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGEPSSIVNVPGESTLRRDFLRLQQENKERSEALRRQQLLQEQQLREQEEYKRQLLAERQKRIEQQKEQRRRLEEQQRREREARRQQEREQRRREQEEKRRLEELERRRKEEEERRRAEEEKRRVEREQEYIRRQLEEEQRHLEVLQQQLLQEQAMLLECRWREMEEHRQAERLQRQLQQEQAYLLSLQHDHRRPHPQHSQQPPPPQQERSKPSFHAPEPKAHYEPADRAREVEDRFRKTNHSSPEAQSKQTGRVLEPPVPSRSESFSNGNSESVHPALQRPAEPQVPVRTTSRSPVLSRRDSPLQGSGQQNSQAGQRNSTSIEPRLLWERVEKLVPRPGSGSSSGSSNSGSQPGSHPGSQSGSGERFRVRSSSKSEGSPSQRLENAVKKPEDKKEVFRPLKPADLTALAKELRAVEDVRPPHKVTDYSSSSEESGTTDEEDDDVEQEGADESTSGPEDTRAASSLNLSNGETESVKTMIVHDDVESEPAMTPSKEGTLIVRQTQSASSTLQKHKSSSSFTPFIDPRLLQISPSSGTTVTSVVGFSCDGMRPEAIRQDPTRKGSVVNVNPTNTRPQSDTPEIRKYKKRFNSEILCAALWGVNLLVGTESGLMLLDRSGQGKVYPLINRRRFQQMDVLEGLNVLVTISGKKDKLRVYYLSWLRNKILHNDPEVEKKQGWTTVGDLEGCVHYKVVKYERIKFLVIALKSSVEVYAWAPKPYHKFMAFKSFGELVHKPLLVDLTVEEGQRLKVIYGSCAGFHAVDVDSGSVYDIYLPTHIQCSIKPHAIIILPNTDGMELLVCYEDEGVYVNTYGRITKDVVLQWGEMPTSVAYIRSNQTMGWGEKAIEIRSVETGHLDGVFMHKRAQRLKFLCERNDKVFFASVRSGGSSQVYFMTLGRTSLLSW

SEQ ID NO: 12 - protein, 859 aa, Artificial Sequence
  Synthetic polypeptide
MACLHETRTPSPSFGGFVSTLSEASMRKLDPDTSDCTPEKDLTPTHVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTMEKLIKKRNVPQKLSPHSKRPDILKTESLLPKLDAALSGVGLPGCPKGPPSPGRSRRGKTRHRKASAKGSCGDLPGLRTAVPPHEPGGPGSPGGLGGGPSAWEACPPALRGLHHDLLLRKMSSSSPDLLSAALGSRGRGATGGAGDPGSPPPARGDTPPSEGSAPGSTSPDSPGGAKGEPPPPVGPGEGVGLLGTGREGTSGRGGSRAGSQHLTPAALLYRAAVTRSQKRGISSEEEEGEVDSEVELTSSQRWPQSLNMRQSLSTFSSENPSDGEEGTASEPSPSGTPEVGSTNTDERPDERSDDMCSQGSEIPLDPPPSEVIPGPEPSSLPIPHQELLRERGPPNSEDSDCDSTELDNSNSVDALRPPASLPP

(19) United States
(12) Patent Application Publication
(10) Pub. No.: US 2022/0193242 A1
(43) Pub. Date: Jun. 23, 2022

(54) IMMUNOPHILIN-DEPENDENT INHIBITORS AND USES THEREOF

(71) Applicant: The Regents of the University of California, Oakland, CA (US)

(72) Inventors: Kevan M. Shokat, San Francisco, CA (US); Ziyang Zhang, San Francisco, CA (US)

(21) Appl. No.: 17/428,080

(22) PCT Filed: Feb. 6, 2020
(86) PCT No.: PCT/US20/17017

§ 371 Date: Aug. 3, 2021

Related U.S. Application Data
(60) Provisional application No. 62/802,665, filed on Feb. 7, 2019.

(51) Int. Cl. A61K 47/55; A61K 47/54; A61K 47/64

(52) U.S. Cl. CPC A61K 47/55; A61K 47/64; A61K 47/545

(57) ABSTRACT

Disclosed herein, inter alia, are immunophilin binding compounds and methods of using the same.

### CROSS-REFERENCES TO RELATED APPLICATIONS

This application claims the benefit of U.S. Provisional Application No. 62/802,665, filed Feb. 7, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. U19 AI109622 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-635001WO_Sequence_Listing_ST25.txt, created Jan. 29, 2020, 150,758 bytes, machine format IBM-PC, MS Windows operating system, is hereby incorporated by reference.

### BACKGROUND

Protein kinases orchestrate an intricate network of cellular signaling events, and their dysregulation are implicated in many human diseases including cancer, autoimmunity and neurodegenerative disorders. Inhibition of aberrant kinases by small molecule ligands proves to be a fruitful therapeutic strategy that remains widely pursued in various disease areas (48 FDA-approved kinase inhibitors as of December 2018). Nonetheless, methods are lacking to allow reversal of the effects of kinase inhibitors or tissue-directed kinase inhibition. These features are highly desirable, as systemic kinase inhibition often is unnecessary and contributes to toxicity. Disclosed herein, inter alia, are solutions to these and other problems in the art.

### BRIEF SUMMARY OF THE INVENTION

In one aspect is provided a compound having the formula: A-L¹-R¹. A is an immunophilin-binding moiety. L¹ is a bond or a covalent linker. R¹ is a kinase inhibitor, a pseudokinase inhibitor, a GTPase inhibitor, a histone-modifying enzyme inhibitor; or a monovalent anti-viral agent; wherein the compound is not

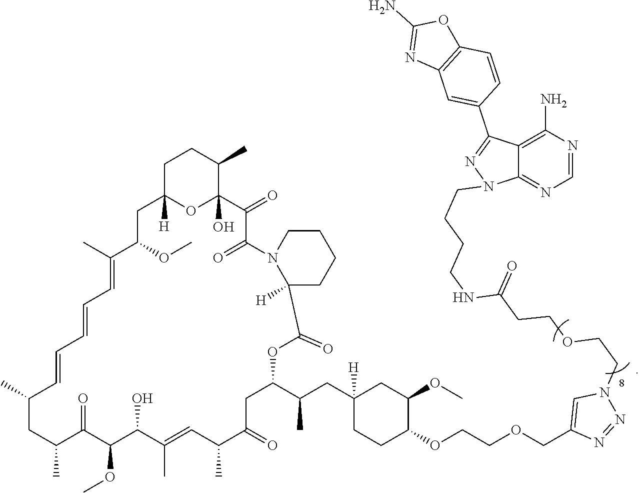

In one aspect is provided a compound as provided herein, including embodiments thereof, wherein the compound is not a calcineurin inhibitor.

In one aspect is provided a pharmaceutical composition including a pharmaceutically acceptable excipient and a compound as provided herein, including embodiments thereof.

In one aspect is provided a method of treating a disease associated with aberrant enzyme activity in a subject in need of such treatment, including administering a compound as provided herein, including embodiments thereof, to the subject.

In one aspect is provided a method of treating a disease in a subject in need of such treatment, including administering a compound as provided herein, including embodiments thereof, to the subject, wherein the disease is a viral disease, cancer, or a neurodegenerative disease.

### BRIEF DESCRIPTION OF THE DRAWINGS

FIGS. 1A-1D. Design and biochemical characterization of a bispecific kinase inhibitor. (FIG. 1A) structures of FK506, dasatinib, and FK-dasatinib. (FIG. 1B) Dose-dependent inhibition of Src, Csk and DDR2 by dasatinib and FK-dasatinib in the absence or presence of supplemented 10 μM recombinant FKBP12 protein Data is the average of two replicates. (FIG. 1C) Profiling of dasatinib and FK-dasatinib against a panel of 485 purified kinases (SelectScreen™) in the presence of FKBP12. Each dot on the scatter plot represent one kinase colored by the extent of their inhibition by dasatinib. (FIG. 1D) Schematic illustration of FK506, dasatinib, FK-dasatinib and FKBP-presented FK-dasatinib.

FIGS. 2A-2C. FK506-Dasatinib forms a stable ternary complex with Src and Dasatinib. (FIG. 2A) A mixture of recombinant Src kinase domain and FKBP12 (1:1.5 molar ratio) was incubated with buffer, dasatinib or FK-dasatinib for 1 h and analyzed by size exclusion chromatography (Superdex 75 10/300). Fractions of 0.5 mL were collected and analyzed by SDS-PAGE. Coomassie-stained gel image of fractions from the FK-dasatinib-treated sample is shown. (FIG. 2B) Thermal denaturation curves of a 1:1 mixture of Src kinase domain and FKBP12 treated with buffer, dasatinib, or FK-dasatinib. (FIG. 2C) Immunoprecipitation of HA-FKBP12 from Jurkat cell lysate (1 mg/mL) treated with DMSO, 1 μM FK506 or 1 μM FK-dasatinib.

FIGS. 3A-3D. FK506-Dasatinib is a potent cell-permeable Src-family kinase inhibitor with long cellular retention time. (FIGS. 3A and 3C) FK-Dasatinib potently inhibits TCR signaling, whereas dasatinib dimers failed to show cellular activity. (FIG. 3B) Profiling of intracellular kinase inhibition by dasatinib and FK-dasatinib using the chemoproteomic probe XO44. Each dot in the scatter plot represents one kinase captured by XO44, and kinases that show statistically significant inhibition (p<0.05, comparing to DMSO-treated samples, Student's t-test) in both dasatinib and FK-dasatinib-treated samples are colored blue. (FIG. 3D) Jurkat cells were treated with dasatinib or FK-dasatinib for 1 h and the drug-containing media were removed and replaced with fresh media. The phosphotyrosine levels were monitored by Western blot at various time points over 24 h.

FIGS. 4A-4C. A general approach to construct FKBP-dependent, programmable kinase inhibitors. (FIG. 4A) Structures of lapatinib and FK-lapatinib, their effects on HER2 signaling and the growth inhibition of SK-BR-3 cells by these compounds. (FIGS. 4B-4C) Structures of GNE7915 and FK-GNE7915 and their inhibition of LRRK2 autophosphorylation.

FIG. 5A. A bispecific molecule built from Dasatinib and a different FKBP ligand (SLF) shows greatly diminished activity (FIG. 5A).

FIGS. 6A-6B. The structure of three dasatinib homodimers (FIG. 6A). Dasatinib homodimers are ineffective at inhibiting Src family kinases (FIG. 6B).

FIG. 7. In cell profiling of kinase inhibition by dasatinib and FK-dasatinib using chemoproteomic probe XO44.

FIG. 8. On-target, off-site drug engagement is an important source of toxicity.

FIG. 9. Building polar components onto existing high-affinity FKBP ligand scaffolds.

FIG. 10. On-target, off-site drug engagement is an important source of toxicity.

FIG. 11A-11B. Immunophilin-dependent kinase inhibitors.

FIG. 12. Immunophilin-dependent kinase inhibitors: Distribution of [3H]FK506 binding sites in brain and peripheral tissues.

FIG. 13A-13C. Potential advantages. (FIG. 13A) Improvement in potency and blocking protein-protein interactions. (FIG. 13B) Possible increase in selectivity and greater intracellular retention. (FIG. 13C) Tissue-specific effects.

FIG. 14. Proof of concept, a rudimentary approach.

FIG. 15. Selected Kinase Inhibitors.

FIG. 16. Case Study 1: Src Kinase Inhibitors. Proof of concept study and brain tumor applications. Immunophilin ligands.

FIG. 17. Design of chimeric kinase inhibitors.

FIG. 18. FK506-Dasatinib hybrid maintains potent FKBP12 binding but attenuated kinase inhibition.

FIG. 19. Activity of 05-022 is dependent on FKBP12.

FIG. 20A-20B. 05-022 has similar target scope to dasatinib. (FIG. 20A) A scatter plot comparing inhibitory activity of dasatinib and 05-022. (FIG. 20B) Percent inhibition of dasatinib and 05-022 against various kinases.

FIG. 21. Src, FKBP12, and 05-022 form a stable ternary complex. Concentrations at injection: Src kinase domain (50 μM), FKBP12 (50 μM), Dasatinib or 05-022 (100 μM).

FIG. 22. Src, FKBP12, and 05-022 form a stable ternary complex. Assay concentrations: Src kinase domain (1 μM), FKBP12 (0 or 1 μM), Dasatinib (1 μM), 05-022 (1 μM), SYPRO Orange (5×).

FIG. 23. Src, FKBP12, and 05-022 form a stable ternary complex. Pulldown was performed with Jurkat cell lysate (1 mg/mL, 200 μL), supplemented with 2 μg HA-FKBP12. Pulldown/wash buffer: 50 mM Tris 7.4, 120 mM NaCl, 1% NP-40, 1 mM EDTA, phosphatase/protease inhibitors.

FIG. 24A-24B. 05-022 potently inhibits p-Tyr signaling in Jurkat cells.

FIG. 25. 05-022 potently inhibits p-Tyr signaling from CD3 crosslinking in Jurkat cells. Jurkat cells (1×10⁶/mL) were treated with the indicated drugs for 1 h, then stimulated with anti-CD3 mAb OKT3 (5 μg/mL) for 5 min. before lysis and analysis.

FIG. 26. Effect of 05-022 is durable after washout. Jurkat cells (1×10⁶/mL) were treated with 100 nM of the indicated compounds for 1 h, then were washed 3 times with PBS and resuspended in culture media. Samples were taken at the indicated time points and lysed immediately.

FIG. 27. 05-022 shows FKBP-dependent growth inhibition of Bcr-Abl Cell Line. K562 (seeding density 5×10⁴/mL) cells, 72 h treatment.

FIG. 28. Generation of FKBP-dependent EGFR inhibitors through multiple rounds of chemical evolution.

FIG. 29. FK-EGFRi displays similar pharmacology to parent inhibitor, albeit slightly less polar. PC-9 or SK-BR-3 cells, 4 h treatment.

FIG. 30. Compound 08-074 demonstrates more potent cellular activity than its parent compound GNE-7915. 3T3 or RAW264.7 cells (MJFF cell line), 2 h treatment.

FIG. 31. Dimerizing KRAS and immunophilins.

FIG. 32. KRAS Inhibitors.

FIG. 33A-33B. Immunophilins accelerate the reaction between KRAS^{G12C} and hybrid ligands. Assay conditions: 4 μM K-Ras+10 μM immunophilin (if indicated)+10 μM Compound; 20 nM HEPES 7.5, 150 mM NaCl, 1 mM MgCl₂, 23° C., 1% DMSO. Percentage labeled was measured by LC-MS analysis of the reaction mixture.

FIG. 34. KRAS^{G12C}, once labeled with 07-014B, forms a stable 1:1 complex with CypA.

FIG. 35. The KRAS.CypA.07-014 complex displays 2-stage melting curve.

FIG. 36A-36B. Limited linker chemistry improves reaction kinetics. Assay conditions: 4 μM K-Ras+10 μM immunophilin (if indicated)+10 μM Compound; 20 nM HEPES 7.5, 150 mM NaCl, 1 mM MgCl₂, 23° C., 1% DMSO.

FIG. 37A-37B. Changing the linker. Assay conditions: 4 μM K-Ras+10 μM immunophilin (if indicated)+10 μM Compound; 20 nM HEPES 7.5, 150 mM NaCl, 1 mM MgCl₂, 23° C., 1% DMSO

FIG. 38. Cellular efficacy, 24 h.

FIG. 39. Cellular efficacy, 24 h.

FIG. 40. Overexpression of either FKBP or CypA did not improve cellular efficacy. H358 cells, treated with inhibitors for another 24 h, 24 h post-transfection.

FIG. 41. The M72C inhibitor scaffold offers a handle to tackle the GTP state.

FIG. 42. Molecules built on the M72C inhibitor scaffold display similar dependence on immunophilins. Assay conditions: 4 μM H-Ras M72C (GDP)+10 μM immunophilin (if indicated)+10 μM Compound; 20 nM HEPES 7.5, 150 mM NaCl, 1 mM MgCl₂, 23° C., 1% DMSO.

FIG. 43A-43B. Molecules built on the M72C inhibitor scaffold display similar dependence on immunophilins.

FIG. 44. HRAS.CypA.08-058 forms a ternary complex, and inhibits Sos-mediated nucleotide exchange. Assay conditions: 1 μM Ras.GDP, 1 μM Mant-GDP, 20 mM EPES 7.5, 150 mM NaCl, 10 mM EDTA or 1 μM Sos. 95A is synonymous to 06-031.

FIG. 45. HRAS.CypA.08-058 ternary complex does not seem to impair Ras.Raf binding. Pulldown conditions: 100 nM KRAS, 50 μg/mL BSA, 20 mM HEPES 7.5, 150 mM NaCl, 5 mM MgCl₂, 1 mM DDT, 1% NP-40. GppNHp loaded proteins were prepared by EDTA-mediated nucleotide exchange.

FIG. 46. Independent Ras.Raf binding TR-FRET assay confirm no significant inhibition of Raf binding.

FIG. 47. Screening novel “dimerizers”.

FIG. 48. Additional brain targets and inhibitors. HGK inhibitor 12k (Bos et al. Cell Chem. Bio 2019), DLK inhibitor 8 (Siu et al. J. Med. Chem. 2018), FKBP-dependent HGK inhibitor, and FKBP-dependent DLK inhibitor.

FIG. 49. PI4K inhibitor of interest (Rutanganira, et al. J. Med. Chem., 2016, 59 (5), 1830-1839) and an example of an FKBP-dependent PI4K inhibitor.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

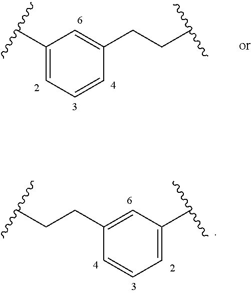 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃,

—CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′,

—C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen,

—SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂,
—R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCH Br₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H,
   —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo,
      halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H,
      —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo,
      halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.;

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido or maleimide groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold, or react with maleimides;

(h) amine or sulfhydryl groups (e.g., present in cysteine), which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g., phosphines) to form, for example, phosphate diester bonds;

(n) azides coupled to alkynes using copper catalyzed cycloaddition click chemistry; and

(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

A “detectable agent” or “detectable moiety” is a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Compounds or functional moieties are “polar” when there are opposing charges (i.e., having partial positive and partial negative charges) from polar bonds arranged asymmetrically. The polarity of a molecule can be measured, for example, by its partition coefficient, P, defined as the ratio of the concentrations of a solute between two immiscible solvents. When one of the solvents is water, the c log P value is a measure of lipophilicity or hydrophobicity. In embodiments, the compound has a c log P of about 5. In embodiments, the compound has a c log P of less than 5. Polarity can also be measured, for example, by its topological polar surface area (PSA), which is the surface sum over all polar atoms, primarily oxygen and nitrogen, also including their attached hydrogen atoms. Molecules with a PSA of greater than 140 Å tend to be poor at permeating cell membranes. In embodiments, for molecules to penetrate the blood-brain barrier, a PSA less than 90 Å is usually necessary. In embodiments, the compound described herein has a PSA between 90 Å and 140 Å. In embodiments, the compound described herein has a PSA between 100 Å and 140 Å.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Co-administer” is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In some embodiments inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule).

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “neurodegenerative disorder” or “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

“Anti-neurodegenerative disease agent” is used in accordance with its plain ordinary meaning and refers to a composition (e.g., compound, drug, antagonist, inhibitor, modulator) capable of inhibiting neurodegeneration. In some embodiments, an anti-neurodegenerative disease agent is an agent identified herein having utility in methods of treating a neurodegenerative disease. In some embodiments, an anti-neurodegenerative disease agent is an agent approved by the FDA or similar regulatory agency of a country other than the USA, for treating a neurodegenerative disease. Examples of anti-neurodegenerative disease agents include, but are not limited to, galantamine, rivastigmine, donepezil, memantine, imatinib, tamibarotene, bexarotene, carmustine, thalidomide, sildenafil, trazodone, clioquinol, nilvadipine, levodopa, pramipexole, repinirole, rotigotine, apomorphine, selegiline, rasagiline, safinamide, amantadine, milotinib, zonisamide, selegiline, methylphenidate, salbutamol, exenatide, tetrabenazine, tiapride, clozapine, olanzapine, risperidone, quetiapine, memantine, mitoxantrone, cyclophosphamide, cladribine, amiloride, ibudilast, mastinib, dolutegravir, abacavir, lamivudine, retigabine, and tamoxifen.

As used herein, the term “metabolic disease” or “metabolic disorder” refers to a disease or condition in which a subject's metabolism or metabolic system (e.g., function of storing or utilizing energy) becomes impaired. Examples of metabolic diseases that may be treated with a compound, pharmaceutical composition, or method described herein include diabetes (e.g., type I or type II), obesity, metabolic syndrome, or a mitochondrial disease (e.g., dysfunction of mitochondria or aberrant mitochondrial function).

The term “cellular component associated disease” (e.g., the cellular component may be a protein, ion, lipid, nucleic acid, nucleotide, amino acid, protein, particle, organelle, cellular compartment, microorganism, virus, vesicle, small molecule, protein complex, protein aggregate, or macromolecule; the disease may be a neurodegenerative disease, cancer, a metabolic disease, autoimmune disease, inflammatory disease, or infectious disease) (also referred to herein as “cellular component related disease”) refers to a disease caused by the cellular component. Other diseases that are associated with aberrant activity or level of the cellular component are well known in the art and determining such diseases are within the skill of a person of skill in the art.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

As a non-limiting example, the compounds described herein can be co-administered with conventional chemotherapeutic agents including alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin, etc.), and the like.

The compounds described herein can also be co-administered with conventional hormonal therapeutic agents including, but not limited to, steroids (e.g., dexamethasone), finasteride, aromatase inhibitors, tamoxifen, and gonadotropin-releasing hormone agonists (GnRH) such as goserelin.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰ or ¹³¹I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

The term “associated” or “associated with” in the context of a substance or substance activity or function associated with a disease (e.g., a protein associated disease, disease associated with a cellular component) means that the disease (e.g., neurodegenerative disease, cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). For example, a neurodegenerative disease associated with a protein aggregate may be a neurodegenerative disease that results (entirely or partially) from aberrant protein aggregation or a neurodegenerative disease wherein a particular symptom of the disease is caused (entirely or partially) by aberrant protein aggregation. As used herein, what is described as being associated with a disease, if a causative agent, could be a target for treatment of the disease. For example, a neurodegenerative disease associated with aberrant protein aggregation or a protein aggregate associated neurodegenerative disease, may be treated with a protein aggregate modulator or protein aggregate targeted autophagy degrader, in the instance where increased protein aggregation causes the neurodegenerative disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g. a monovalent form of an agent listed above).

“Chemotherapeutic” or “chemotherapeutic agent” is used in accordance with its plain ordinary meaning and refers to a chemical composition or compound having antineoplastic properties or the ability to inhibit the growth or proliferation of cells.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond. In some embodiments, the electrophilic substituent of the compound is capable of reacting with a cysteine residue. In some embodiments, the electrophilic substituent is capable of forming a covalent bond with a cysteine residue and may be referred to as a “covalent cysteine modifier moiety” or “covalent cysteine modifier substituent.” The covalent bond formed between the electrophilic substituent and the sulfhydryl group of the cysteine may be a reversible or irreversible bond. In some embodiments, the electrophilic substituent of the compound is capable of reacting with a lysine residue. In some embodiments, the electrophilic substituent of the compound is capable of reacting with a serine residue. In some embodiments, the electrophilic substituent of the compound is capable of reacting with a methionine residue.

“Nucleophilic” as used herein refers to a chemical group that is capable of donating electron density.

An amino acid residue in a protein “corresponds” to a given residue when it occupies the same essential structural position within the protein as the given residue. Instead of a primary sequence alignment, a three dimensional structural alignment can also be used, e.g., where the structure of the selected protein is aligned for maximum correspondence with the human protein and the overall structures compared. In this case, an amino acid that occupies the same essential position as a specified amino acid in the structural model is said to correspond to the specified residue.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes. In embodiments, the protein aggregate is TDP43, HTT, APP, SNCA, or MAPT. In embodiments, the protein aggregate includes the protein Beta amyloid, Amyloid precursor protein, IAPP, Alpha-synuclein, PrPSc, PrPSc, Huntingtin, Calcitonin, Atrial natriuretic factor, Apolipoprotein A1, Serum amyloid A, Medin, Prolactin, Transthyretin, Lysozyme, Beta-2 microglobulin, Gelsolin, Keratoepithelin, Beta amyloid, Cystatin, Immunoglobulin light chain AL, TDP43, or S-IBM.

The term “vesicle” is used in accordance with its plain ordinary meaning and refers to a small membrane enclosed compartment within a cell. Vesicles are typically involved in transport, buoyancy control, or enzyme storage within a cell. Some vesicles, for example a lysosome, may include enzymes, proteins, polysaccharides, lipids, nucleic acids, or organelles within the compartment. Vesicles are typically formed within cells as a result of exocytosis or phagocytosis, however some vesicles are formed at the Golgi complex and transported to the cell membrane. Vesicles may be unilamellar or multilamellar.

The term “small molecule” refers to a low molecular weight organic compound that may regulate a biological process. In embodiments, the small molecule is a compound that weighs less than 900 daltons. In embodiments, the small molecule weighs less than 800 daltons. In embodiments, the small molecule weighs less than 700 daltons. In embodiments, the small molecule weighs less than 600 daltons. In embodiments, the small molecule weighs less than 500 daltons. In embodiments, the small molecule weighs less than 450 daltons. In embodiments, the small molecule weighs less than 400 daltons.

The term “mTOR” refers to the protein “mechanistic target of rapamycin (serine/threonine kinase)” or “mammalian target of rapamycin.” The term “mTOR” may refer to the nucleotide sequence or protein sequence of human mTOR (e.g., Entrez 2475, Uniprot P42345, RefSeq NM_004958, or RefSeq NP_004949). The term “mTOR” includes both the wild-type form of the nucleotide sequences or proteins as well as any mutants thereof. In some embodiments, “mTOR” is wild-type mTOR. In some embodiments, “mTOR” is one or more mutant forms. The term “mTOR” XYZ refers to a nucleotide sequence or protein of a mutant mTOR wherein the Y numbered amino acid of mTOR that normally has an X amino acid in the wildtype, instead has a Z amino acid in the mutant. In embodiments, an mTOR is the human mTOR. In embodiments, the mTOR has the following amino acid sequence:

(SEQ ID NO: 1)
MLGTGPAAATTAATTSSNVSVLQQFASGLKSRNEETRAKAAKELQHYVTMELREMSQEEST
RFYDQLNHHIFELVSSSDANERKGGILAIASLIGVEGGNATRIGRFANYLRNLLPSNDPVVME
MASKAIGRLAMAGDTFTAEYVEFEVKRALEWLGADRNEGRRHAAVLVLRELAISVPTFFFQ
QVQPFFDNIFVAVWDPKQAIREGAVAALRACLILTTQREPKEMQKPQWYRHTFEEAEKGFDE
TLAKEKGMNRDDRIHGALLILNELVRISSMEGERLREEMEEITQQQLVHDKYCKDLMGFGTK
PRHITPFTSFQAVQPQQSNALVGLLGYSSHQGLMGFGTSPSPAKSTLVESRCCRDLMEEKFDQ
VCQWVLKCRNSKNSLIQMTILNLLPRLAAFRPSAFTDTQYLQDTMNHVLSCVKKEKERTAAF
QALGLLSVAVRSEFKVYLPRVLDIIRAALPPKDFAHKRQKAMQVDATVFTCISMLARAMGPG
IQQDIKELLEPMLAVGLSPALTAVLYDLSRQIPQLKKDIQDGLLKMLSLVLMHKPLRHPGMP
KGLAHQLASPGLTTLPEASDVGSITLALRTLGSFEFEGHSLTQFVRHCADHFLNSEHKEIRME
AARTCSRLLTPSIHLISGHAHVVSQTAVQVVADVLSKLLVVGITDPDPDIRYCVLASLDERFD
AHLAQAENLQALFVALNDQVFEIRELAICTVGRLSSMNPAFVMPFLRKMLIQILTELEHSGIG
RIKEQSARMLGHLVSNAPRLIRPYMEPILKALILKLKDPDPDPNPGVINNVLATIGELAQVSGL
EMRKWVDELFIIIMDMLQDSSLLAKRQVALWTLGQLVASTGYVVEPYRKYPTLLEVLLNFL
KTEQNQGTRREAIRVLGLLGALDPYKHKVNIGMIDQSRDASAVSLSESKSSQDSSDYSTSEML
VNMGNLPLDEFYPAVSMVALMRIFRDQSLSHHHTMVVQAITFIFKSLGLKCVQFLPQVMPTF
LNVIRVCDGAIREFLFQQLGMLVSFVKSHIRPYMDEIVTLMREFWVMNTSIQSTIILLIEQIVVA
LGGEFKLYLPQLIPHMLRVFMHDNSPGRIVSIKLLAAIQLFGANLDDYLHLLLPPIVKLFDAPE
APLPSRKAALETVDRLTESLDFTDYASRIIHPIVRTLDQSPELRSTAMDTLSSLVFQLGKKYQIF
IPMVNKVLVRHRINHQRYDVLICRIVKGYTLADEEEDPLIYQHRMLRSGQGDALASGPVETG
PMKKLHVSTINLQKAWGAARRVSKDDWLEWLRRLSLELLKDSSSPSLRSCWALAQAYNPM
ARDLFNAAFVSCWSELNEDQQDELIRSIELALTSQDIAEVTQTLLNLAEFMEHSDKGPLPLRD
DNGIVLLGERAAKCRAYAKALHYKELEFQKGPTPAILESLISINNKLQQPEAAAGVLEYAMK
HFGELEIQATWYEKLHEWEDALVAYDKKMDTNKDDPELMLGRMRCLEALGEWGQLHQQC
CEKWTLVNDETQAKMARMAAAAAWGLGQWDSMEEYTCMIPRDTHDGAFYRAVLALHQD
LFSLAQQCIDKARDLLDAELTAMAGESYSRAYGAMVSCHMLSELEEVIQYKLVPERREIIRQI
WWERLQGCQRIVEDWQKILMVRSLVVSPHEDMRTWLKYASLCGKSGRLALAHKTLVLLLG
VDPSRQLDHPLPTVHPQVTYAYMKNMWKSARKIDAFQHMQHFVQTMQQQAQHAIATEDQ
QHKQELHKLMARCFLKLGEWQLNLQGINESTIPKVLQYYSAATEHDRSWYKAWHAWAVM
NFEAVLHYKHQNQARDEKKKLRHASGANITNATTAATTAATATTTASTEGSNSESEAESTEN
SPTPSPLQKKVTEDLSKTLLMYTVPAVQGFFRSISLSRGNNLQDTLRVLTLWFDYGHWPDVN
EALVEGVKAIQIDTWLQVIPQLIARIDTPRPLVGRLIHQLLTDIGRYHPQALIYPLTVASKSTTT
ARHNAANKILKNMCEHSNTLVQQAMMVSEELIRVAILWHEMWHEGLEEASRLYFGERNVK
GMFEVLEPLHAMMERGPQTLKETSFNQAYGRDLMEAQEWCRKYMKSGNVKDLTQAWDLY
YHVFRRISKQLPQLTSLELQYVSPKLLMCRDLELAVPGTYDPNQPIIRIQSIAPSLQVITSKQRP
RKLTLMGSNGHEFVFLLKGHEDLRQDERVMQLFGLVNTLLANDPTSLRKNLSIQRYAVIPLS
TNSGLIGWVPHCDTLHALIRDYREKKKILLNIEHRIMLRMAPDYDHLTLMQKVEVFEHAVNN
TAGDDLAKLLWLKSPSSEVWFDRRTNYTRSLAVMSMVGYILGLGDRHPSNLMLDRLSGKIL
HIDFGDCFEVAMTREKFPEKIPFRLTRMLTNAMEVTGLDGNYRITCHTVMEVLREHKDSVM
AVLEAFVYDPLLNWRLMDTNTKGNKRSRTRTDSYSAGQSVEILDGVELGEPAHKKTGTTVP
ESIHSFIGDGLVKPEALNKKAIQIINRVRDKLTGRDFSHDDTLDVPTQVELLIKQATSHENLCQ
CYIGWCPFW.

The term “mTORC1” refers to the protein complex including mTOR and Raptor (regulatory-associated protein of mTOR). mTORC1 may also include MLST8 (mammalian lethal with SEC13 protein 8), PRAS40, and/or DEPTOR. mTORC1 may function as a nutrient/energy/redox sensor and regulator of protein synthesis. The term “mTORC1 pathway” or “mTORC1 signal transduction pathway” refers to a cellular pathway including mTORC1. An mTORC1 pathway includes the pathway components upstream and downstream from mTORC1. An mTORC1 pathway is a signaling pathway that is modulated by modulation of mTORC1 activity. In embodiments, an mTORC1 pathway is a signaling pathway that is modulated by modulation of mTORC1 activity but not by modulation of mTORC2 activity. In embodiments, an mTORC1 pathway is a signaling pathway that is modulated to a greater extent by modulation of mTORC1 activity than by modulation of mTORC2 activity.

The term “mTORC2” refers to the protein complex including mTOR and RICTOR (rapamycin-insensitive companion of mTOR). mTORC2 may also include GβL, mSIN1 (mammalian stress-activated protein kinase interacting protein 1), Protor 1/2, DEPTOR, TTI1, and/or TEL2. mTORC2 may regulate cellular metabolism and the cytoskeleton. The term “mTORC2 pathway” or “mTORC2 signal transduction pathway” refers to a cellular pathway including mTORC2. An mTORC2 pathway includes the pathway components upstream and downstream from mTORC2. An mTORC2 pathway is a signaling pathway that is modulated by modulation of mTORC2 activity. In embodiments, an mTORC2 pathway is a signaling pathway that is modulated by modulation of mTORC2 activity but not by modulation of mTORC1 activity. In embodiments, an mTORC2 pathway is a signaling pathway that is modulated to a greater extent by modulation of mTORC2 activity than by modulation of mTORC1 activity.

The term “pseudokinase” is used in accordance with its well understood meaning in Biology and Chemistry and refers to proteins that are variants of kinases (e.g., having similar or identical protein structures or folds) that are catalytically deficient in kinase enzymatic activity.

The term “GTPase” is used in accordance with its well understood meaning in Biology and Chemistry and refers to hydrolase enzymes capable of binding and hydrolyzing GTP.

The term “histone modifying enzyme is used in accordance with its well understood meaning in Biology and Chemistry and refers to proteins that are capable of modifying histones at one or more of various sites. In embodiments a histone modifying enzyme is an enzyme capable of acetylation, methylation, demethylation, phosphorylation, ubiquitination, sumoylation, ADP-ribosylation, deamination, and/or proline isomerization; all of one or more histone proteins. In embodiments, the histone modifying enzyme is a histone deacetylase, histone methyltransferase, or histone acetyltransferase. In embodiments, the histone modifying enzyme is SETD3.

The term “kinase inhibitor” refers to an agent (e.g., small molecule, nucleic acid, protein, or antibody) that can reduce the activity or level of a kinase.

The term “pseudokinase inhibitor” refers to an agent (e.g., small molecule, nucleic acid, protein, or antibody) that can reduce the activity or level of a pseudokinase.

The term “GTPase inhibitor” refers to an agent (e.g., small molecule, nucleic acid, protein, or antibody) that can reduce the activity or level of a GTPase.

The term “histone modifying enzyme inhibitor” refers to an agent (e.g., small molecule, nucleic acid, protein, or antibody) that can reduce the activity or level of a histone modifying enzyme.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “viral disease” is an infection that occurs when an organism's body is invaded by pathogenic viruses and infectious virus particles attach to and enter susceptible cells.

The term “anti-viral agent” refers to an agent (e.g., small molecule, nucleic acid, protein, or antibody) that can reduce the activity or level of a virus (e.g., in a subject or patient).

The term “FKBP” refers to a protein Peptidyl-prolyl cis-trans isomerase. For non-limiting examples of FKBP, see Cell Mol Life Sci. 2013 September; 70(18):3243-75. In embodiments, “FKBP” refers to “FKBP-12” or “FKBP 12” or “FKBP1A”. In embodiments, “FKBP” refers to the human protein. Included in the term “FKBP” is the wildtype and mutant forms of the protein. In embodiments, “FKBP” refers to the wildtype human protein. In embodiments, “FKBP” refers to the wildtype human nucleic acid. In embodiments, the FKBP is a mutant FKBP. In embodiments, the mutant FKBP is associated with a disease that is not associated with wildtype FKBP. In embodiments, the FKBP includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype FKBP. In embodiments, FKBP refers to human AIP, AIPL1, FKBP1A, FKBP1B, FKBP2, FKBP3, FKBP5, FKBP6, FKBP7, FKBP8, FKBP9, FKBP9L, FKBP10, FKBP11, FKBP14, FKBP15, FKBP52, FKBP51, or LOC541473.

The term “FKBP-12” or “FKBP 12” or “FKBP1A” refers to the protein “Peptidyl-prolyl cis-trans isomerase FKBP1A”. In embodiments, ““FKBP-12” or “FKBP 12” or “FKBP1A” refers to the human protein. Included in the term “FKBP-12” or “FKBP 12” or “FKBP1A” are the wildtype and mutant forms of the protein. In embodiments, “FKBP-12” or “FKBP 12” or “FKBP1A” refers to the protein associated with Entrez Gene 2280, OMIM 186945, UniProt P62942, and/or RefSeq (protein) NP_000792. In embodiments, the reference numbers immediately above refer to the protein, and associated nucleic acids, known as of the date of filing of this application. In embodiments, “FKBP-12” or “FKBP 12” or “FKBP1A” refers to the wildtype human protein. In embodiments, “FKBP-12” or “FKBP 12” or “FKBP1A” refers to the wildtype human nucleic acid. In embodiments, the FKBP-12 is a mutant FKBP-12. In embodiments, the mutant FKBP-12 is associated with a disease that is not associated with wildtype FKBP-12. In embodiments, the FKBP-12 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype FKBP-12. In embodiments, the FKBP-12 has the protein sequence corresponding to RefSeq NP_000792.1. In embodiments, the FKBP-12 has the protein sequence corresponding to RefSeq NM_000801.5.

The term “Calcineurin” refers to a protein, which is a calcium and calmodulin dependent serine/threonine protein phosphatase, also known as a protein phosphatase 3, and calcium-dependent serine-threonine phosphatase. Calcineurin is a heterodimer of a 61-kD calmodulin-binding catalytic subunit, calcineurin A and a 19-kD Ca2+-binding regulatory subunit, calcineurin B. There are three isozymes of the catalytic subunit, each encoded by a separate gene (PPP3CA, PPP3CB, and PPP3CC) and two isoforms of the regulatory, also encoded by separate genes (PPP3R1, PPP3R2).

The term “immunophilins” refers to cytosolic peptidyl-prolyl isomerases that catalyze the interconversion between the cis and trans isomers of peptide bonds containing the amino acid proline. Immunophilins can be classified into two main families: “cyclosporin-binding cyclophilins” and “FK506-binding proteins.” Immunophilins act as receptors for immunosuppressive drugs, such as cyclosporin and tacrolimus (or FK506), which inhibit the prolyl isomerase activity of immunophilins. In embodiments, the compound described herein is an immunophilin-binding compound. In embodiments, the compound includes an immunophilin-binding moiety.

The term “cyclophilin” refers to a family of proteins that bind to cyclosporin, which is an immunosuppressant usually used to suppress rejection after internal organ transplants. Cyclophilins have peptidyl prolyl isomerase activity. In embodiments, the compound described herein is a cyclophilin-binding compound. In embodiments, the compound includes a cyclophilin-binding moiety.

The term “FK506-binding protein” or “FKBP” refers to a family of proteins that have peptidyl prolyl isomerase activity. FKBP12 is notable in humans for binding tacrolimus (or FK506), which is an immunosuppressant used in treating subjects after organ transplant as well as subjects suffering from autoimmune disorders. Both the FKBP-FK506 complex and the cyclosporin-cyclophilin complex inhibit calcineurin, thus blocking signal transduction in the T-lymphocyte transduction pathway.

The term “EGFR” or “ErbB-1” or “HER1” refers to the protein “Epidermal growth factor receptor”. In embodiments, “EGFR” or “ErbB-1” or “HER1” refers to the human protein. Included in the term “EGFR” or “ErbB-1” or “HER1” are the wildtype and mutant forms of the protein. In embodiments, “EGFR” or “ErbB-1” or “HER1” refers to the protein associated with Entrez Gene 1956, OMIM 131550, UniProt P00533, and/or RefSeq (protein) NP_005219, RefSeq (protein) NP_958439, RefSeq (protein) NP_958440, or RefSeq (protein) NP_958441. In embodiments, the reference numbers immediately above refer to the protein, and associated nucleic acids, known as of the date of filing of this application. In embodiments, “EGFR” or “ErbB-1” or “HER1” refers to the wildtype human protein. In embodiments, “EGFR” or “ErbB-1” or “HER1” refers to the wildtype human nucleic acid. In embodiments, the EGFR is a mutant EGFR. In embodiments, the mutant EGFR is associated with a disease that is not associated with wildtype EGFR. In embodiments, the mutant EGFR is associated with cancer. In embodiments, the EGFR includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype EGFR. In embodiments, the EGFR has the protein sequence corresponding to RefSeq NP_005219.2. In embodiments, the EGFR has the protein sequence corresponding to RefSeq NM_005219.2. In embodiments, the EGFR has the following amino acid sequence:

(SEQ ID NO: 2)
MRPSGTAGAALLALLAALCPASRALEEKKVCQGTSNKLTQLGTFEDHFLSLQRMFNNCEVV
LGNLEITYVQRNYDLSFLKTIQEVAGYVLIALNTVERIPLENLQIIRGNMYYENSYALAVLSN
YDANKTGLKELPMRNLQEILHGAVRFSNNPALCNVESIQWRDIVSSDFLSNMSMDFQNHLGS
CQKCDPSCPNGSCWGAGEENCQKLTKIICAQQCSGRCRGKSPSDCCHNQCAAGCTGPRESDC
LVCRKFRDEATCKDTCPPLMLYNPTTYQMDVNPEGKYSFGATCVKKCPRNYVVTDHGSCV
RACGADSYEMEEDGVRKCKKCEGPCRKVCNGIGIGEFKDSLSINATNIKHFKNCTSISGDLHI
LPVAFRGDSFTHTPPLDPQELDILKTVKEITGFLLIQAWPENRTDLHAFENLEIIRGRTKQHGQ
FSLAVVSLNITSLGLRSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFVENSECIQCHPEC
LPQAMNITCTGRGPDNCIQCAHYIDGPHCVKTCPAGVMGENNTLVWKYADAGHVCHLCHN
CTYGCTGPGLEGCPTNGPKIPSIATGMVGALLLLLVVALGIGLFMRRRHIVRKRTLRRLLQER
ELVEPLTPSGEAPNQALLRILKETEFKKIKVLGSGAFGTVYKGLWIPEGEKVKIPVAIKELREA
TSPKANKEILDEAYVMASVDNPHVCRLLGICLTSTVQLITQLMPFGCLLDYVREHKDNIGSQY
LLNWCVQIAKGMNYLEDRRLVHRDLAARNVLVKTPQHVKITDFGLAKLLGAEEKEYHAEG
GKVPIKWMALESILHRIYTHQSDVWSYGVTVWELMTFGSKPYDGIPASEISSILEKGERLPQPP
ICTIDVYMIMVKCWMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACIDRNGLQSCPIKEDS
FLQRYSSDPTGALTEDSIDDTFLPVPEYINQSVPKRPAGSVQNPVYHNQPLNPAPSRDPHYQD
PHSTAVGNPEYLNTVQPTCVNSTFDSPAHWAQKGSHQISLDNPDYQQDFFPKEAKPNGIFKG
STAENAEYLRVAPQSSEFIGA.

The term “HER2” or “ErbB-2” or “ERBB2” refers to the protein “human epidermal growth factor receptor 2”. In embodiments, “HER2” or “ErbB-2” or “ERBB2” refers to the protein “receptor tyrosine-protein kinase erbB-2”. In embodiments, “HER2” or “ErbB-2” or “ERBB2” refers to the human protein. Included in the term “HER2” or “ErbB-2” or “ERBB2” are the wildtype and mutant forms of the protein. In embodiments, “HER2” or “ErbB-2” or “ERBB2” refers to the protein associated with Entrez Gene 2064, OMIM 164870, UniProt P04626, and/or RefSeq (protein) NP_004439. In embodiments, the reference numbers immediately above refer to the protein, and associated nucleic acids, known as of the date of filing of this application. In embodiments, “HER2” or “ErbB-2” or “ERBB2” refers to the wildtype human protein. In embodiments, “HER2” or “ErbB-2” or “ERBB2” refers to the wildtype human nucleic acid. In embodiments, the HER2 protein is a mutant HER2 protein. In embodiments, the mutant HER2 protein is associated with a disease that is not associated with wildtype HER-2. In embodiments, the mutant HER-2 is associated with cancer. In embodiments, the mutant HER-2 is associated with breast cancer. In embodiments, the HER-2 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype HER-2. In embodiments, the HER-2 protein has the protein sequence corresponding to RefSeq NP_004439.2. In embodiments, the HER-2 protein has the protein sequence corresponding to RefSeq NM_004448.3. In embodiments, the HER2 has the following amino acid sequence:

(SEQ ID NO: 3)
MELAALCRWGLLLALLPPGAASTQVCTGTDMKLRLPASPETHLDMLRHLYQGCQVVQGNL
ELTYLPTNASLSFLQDIQEVQGYVLIAHNQVRQVPLQRLRIVRGTQLFEDNYALAVLDNGDP
LNNTTPVTGASPGGLRELQLRSLTEILKGGVLIQRNPQLCYQDTILWKDIFHKNNQLALTLIDT
NRSRACHPCSPMCKGSRCWGESSEDCQSLTRTVCAGGCARCKGPLPTDCCHEQCAAGCTGP
KHSDCLACLHFNHSGICELHCPALVTYNTDTFESMPNPEGRYTFGASCVTACPYNYLSTDVG
SCTLVCPLHNQEVTAEDGTQRCEKCSKPCARVCYGLGMEHLREVRAVTSANIQEFAGCKKIF
GSLAFLPESFDGDPASNTAPLQPEQLQVFETLEEITGYLYISAWPDSLPDLSVFQNLQVIRGRIL
HNGAYSLTLQGLGISWLGLRSLRELGSGLALIHHNTHLCFVHTVPWDQLFRNPHQALLHTAN
RPEDECVGEGLACHQLCARGHCWGPGPTQCVNCSQFLRGQECVEECRVLQGLPREYVNAR
HCLPCHPECQPQNGSVTCFGPEADQCVACAHYKDPPFCVARCPSGVKPDLSYMPIWKFPDEE
GACQPCPINCTHSCVDLDDKGCPAEQRASPLTSIISAVVGILLVVVLGVVFGILIKRRQQKIRK
YTMRRLLQETELVEPLTPSGAMPNQAQMRILKETELRKVKVLGSGAFGTVYKGIWIPDGENV
KIPVAIKVLRENTSPKANKEILDEAYVMAGVGSPYVSRLLGICLTSTVQLVTQLMPYGCLLDH
VRENRGRLGSQDLLNWCMQIAKGMSYLEDVRLVHRDLAARNVLVKSPNHVKITDFGLARL
LDIDETEYHADGGKVPIKWMALESILRRRFTHQSDVWSYGVTVWELMTFGAKPYDGIPAREI
PDLLEKGERLPQPPICTIDVYMIMVKCWMIDSECRPRFRELVSEFSRMARDPQRFVVIQNEDL
GPASPLDSTFYRSLLEDDDMGDLVDAEEYLVPQQGFFCPDPAPGAGGMVHHRHRSSSTRSG
GGDLTLGLEPSEEEAPRSPLAPSEGAGSDVFDGDLGMGAAKGLQSLPTHDPSPLQRYSEDPTV
PLPSETDGYVAPLTCSPQPEYVNQPDVRPQPPSPREGPLPAARPAGATLERPKTLSPGKNGVV
KDVFAFGGAVENPEYLTPQGGAAPQPHPPPAFSPAFDNLYYWDQDPPERGAPPSTFKGTPTA
ENPEYLGLDVPV.

The term “LRRK” or “LRKK2” or “dardarin” refers to the protein “Leucine-rich repeat kinase 2”. In embodiments, “LRRK” or “LRKK2” or “dardarin” refers to the human protein. Included in the term “LRRK” or “LRKK2” or “dardarin” are the wildtype and mutant forms of the protein. In embodiments, “LRRK” or “LRKK2” or “dardarin” refers to the protein associated with Entrez Gene 120892, OMIM 609007, UniProt Q5S007, and/or RefSeq (protein) NP_940980. In embodiments, the reference numbers immediately above refer to the protein, and associated nucleic acids, known as of the date of filing of this application. In embodiments, “LRRK” or “LRKK2” or “dardarin” refers to the wildtype human protein. In embodiments, “LRRK” or “LRKK2” or “dardarin” refers to the wildtype human nucleic acid. In embodiments, the LRKK2 is a mutant LRKK2 protein. In embodiments, the mutant LRKK2 is associated with a disease that is not associated with wildtype LRKK2. In embodiments, the LRKK2 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype LRKK2. In embodiments, the LRKK2 protein has the protein sequence corresponding to RefSeq NP_940980.3. In embodiments, the LRKK2 protein has the protein sequence corresponding to RefSeq NM_198578.3. In embodiments, the LRRK2 has the following amino acid sequence:

(SEQ ID NO: 4)
MASGSCQGCEEDEETLKKLIVRLNNVQEGKQIETLVQILEDLLVFTYSERASKLFQGKNIHVP
LLIVLDSYMRVASVQQVGWSLLCKLIEVCPGTMQSLMGPQDVGNDWEVLGVHQLILKMLT
VHNASVNLSVIGLKTLDLLLTSGKITLLILDEESDIFMLIFDAMHSFPANDEVQKLGCKALHVL
FERVSEEQLTEFVENKDYMILLSALTNFKDEEEIVLHVLHCLHSLAIPCNNVEVLMSGNVRCY
NIVVEAMKAFPMSERIQEVSCCLLHRLTLGNFFNILVLNEVHEFVVKAVQQYPENAALQISAL
SCLALLTETIFLNQDLEEKNENQENDDEGEEDKLFWLEACYKALTWHRKNKHVQEAACWA
LNNLLMYQNSLHEKIGDEDGHFPAHREVMLSMLMHSSSKEVFQASANALSTLLEQNVNFRK
ILLSKGIHLNVLELMQKHIHSPEVAESGCKMLNHLFEGSNTSLDIMAAVVPKILTVMKRHETS
LPVQLEALRAILHFIVPGMPEESREDTEFHHKLNMVKKQCFKNDIHKLVLAALNRFIGNPGIQ
KCGLKVISSIVHFPDALEMLSLEGAMDSVLHTLQMYPDDQEIQCLGLSLIGYLITKKNVFIGTG
HLLAKILVSSLYRFKDVAEIQTKGFQTILAILKLSASFSKLLVHHSFDLVIFHQMSSNIMEQKD
QQFLNLCCKCFAKVAMDDYLKNVMLERACDQNNSIMVECLLLLGADANQAKEGSSLICQV
CEKESSPKLVELLLNSGSREQDVRKALTISIGKGDSQIISLLLRRLALDVANNSICLGGFCIGKV
EPSWLGPLFPDKTSNLRKQTNIASTLARMVIRYQMKSAVEEGTASGSDGNFSEDVLSKFDEW
TFIPDSSMDSVFAQSDDLDSEGSEGSFLVKKKSNSISVGEFYRDAVLQRCSPNLQRHSNSLGPI
FDHEDLLKRKRKILSSDDSLRSSKLQSHMRHSDSISSLASEREYITSLDLSANELRDIDALSQKC
CISVHLEHLEKLELHQNALTSFPQQLCETLKSLTHLDLHSNKFTSFPSYLLKMSCIANLDVSRN
DIGPSVVLDPTVKCPTLKQFNLSYNQLSFVPENLTDVVEKLEQLILEGNKISGICSPLRLKELKI
LNLSKNHISSLSENFLEACPKVESFSARMNFLAAMPFLPPSMTILKLSQNKFSCIPEAILNLPHL
RSLDMSSNDIQYLPGPAHWKSLNLRELLFSHNQISILDLSEKAYLWSRVEKLHLSHNKLKEIPP
EIGCLENLTSLDVSYNLELRSFPNEMGKLSKIWDLPLDELHLNFDFKHIGCKAKDIIRFLQQRL
KKAVPYNRMKLMIVGNTGSGKTTLLQQLMKTKKSDLGMQSATVGIDVKDWPIQIRDKRKR
DLVLNVWDFAGREEFYSTHPHFMTQRALYLAVYDLSKGQAEVDAMKPWLFNIKARASSSP
VILVGTHLDVSDEKQRKACMSKITKELLNKRGFPAIRDYHFVNATEESDALAKLRKTIINESL
NFKIRDQLVVGQLIPDCYVELEKIILSERKNVPIEFPVIDRKRLLQLVRENQLQLDENELPHAV
HFLNESGVLLHFQDPALQLSDLYFVEPKWLCKIMAQILTVKVEGCPKHPKGIISRRDVEKFLS
KKRKFPKNYMSQYFKLLEKFQIALPIGEEYLLVPSSLSDHRPVIELPHCENSEIIIRLYEMPYFP
MGFWSRLINRLLEISPYMLSGRERALRPNRMYWRQGIYLNWSPEAYCLVGSEVLDNHPESFL
KITVPSCRKGCILLGQVVDHIDSLMEEWFPGLLEIDICGEGETLLKKWALYSFNDGEEHQKILL
DDLMKKAEEGDLLVNPDQPRLTIPISQIAPDLILADLPRNIMLNNDELEFEQAPEFLLGDGSFG
SVYRAAYEGEEVAVKIFNKHTSLRLLRQELVVLCHLHHPSLISLLAAGIRPRMLVMELASKGS
LDRLLQQDKASLTRTLQHRIALHVADGLRYLHSAMIIYRDLKPHNVLLFTLYPNAAIIAKIAD
YGIAQYCCRMGIKTSEGTPGFRAPEVARGNVIYNQQADVYSFGLLLYDILTTGGRIVEGLKFP
NEFDELEIQGKLPDPVKEYGCAPWPMVEKLIKQCLKENPQERPTSAQVFDILNSAELVCLTRR
ILLPKNVIVECMVATHHNSRNASIWLGCGHTDRGQLSFLDLNTEGYTSEEVADSRILCLALVH
LPVEKESWIVSGTQSGTLLVINTEDGKKRHTLEKMTDSVTCLYCNSFSKQSKQKNFLLVGTA
DGKLAIFEDKTVKLKGAAPLKILNIGNVSTPLMCLSESTNSTERNVMWGGCGTKIFSFSNDFTI
QKLIETRTSQLFSYAAFSDSNIITVVVDTALYIAKQNSPVVEVWDKKTEKLCGLIDCVHFLRE
VMVKENKESKHKMSYSGRVKTLCLQKNTALWIGTGGGHILLLDLSTRRLIRVIYNFCNSVRV
MMTAQLGSLKNVMLVLGYNRKNTEGTQKQKEIQSCLTVWDINLPHEVQNLEKHIEVRKELA
EKMRRTSVE.

The term “KRAS” or “K-Ras” or “Ki-ras” refers to the protein “Kirsten Rat Sarcoma”. In embodiments, “KRAS” or “K-Ras” or “Ki-ras” refers to the human protein. Included in the term “KRAS” or “K-Ras” or “Ki-ras” are the wildtype and mutant forms of the protein. In embodiments, “KRAS” or “K-Ras” or “Ki-ras” refers to the protein associated with Entrez Gene 3845, OMIM 190070, UniProt P01116, and/or RefSeq (protein) NP_004976, RefSeq (protein) NP_004976.2, or RefSeq (protein) NP_203524. In embodiments, the reference numbers immediately above refer to the protein, and associated nucleic acids, known as of the date of filing of this application. In embodiments, “KRAS” or “K-Ras” or “Ki-ras” refers to the wildtype human protein. In embodiments, “KRAS” or “K-Ras” or “Ki-ras” refers to the wildtype human nucleic acid. In embodiments, the KRAS is a mutant KRAS protein. In embodiments, the mutant KRAS is associated with a disease that is not associated with wildtype KRAS. In embodiments, the KRAS includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype KRAS. In embodiments, the KRAS protein has the protein sequence corresponding to RefSeq NP_004976.2. In embodiments, the KRAS protein has the protein sequence corresponding to RefSeq NM_004985.4. In embodiments, the KRAS has the following amino acid sequence:

In embodiments, the KRAS has the following amino
acid sequence:
(SEQ ID NO: 5)
MTEYKLVVVGAGGVGKSALTIQLIQNHFVDEYDPTIEDSYRKQVVIDGETC
LLDILDTAGQEEYSAMRDQYMRTGEGFLCVFAINNTKSFEDIHHYREQIKR
VKDSEDVPMVLVGNKCDLPSRTVDTKQAQDLARSYGIPFIETSAKTRQRVE
DAFYTLVREIRQYRLKKISKEEKTPGCVKIKKCIIM.
In embodiments, the KRAS has the following amino
acid sequence:
(SEQ ID NO: 6)
MTEYKLVVVGAGGVGKSALTIQLIQNHFVDEYDPTIEDSYRKQVVIDGETC
LLDILDTAGQEEYSAMRDQYMRTGEGFLCVFAINNTKSFEDIHHYREQIKR
VKDSEDVPMVLVGNKCDLPSRTVDTKQAQDLARSYGIPFIETSAKTRQGVD
DAFYTLVREIRKHKEKMSKDGKKKKKKSKTKCVIM.

The term “PI4KA” or “PI4K-ALPHA” refers to the protein “Phosphatidylinositol 4-kinase alpha”. In embodiments, “PI4KA” or “PI4K-ALPHA” refers to the human protein. Included in the term “PI4KA” or “PI4K-ALPHA” are the wildtype and mutant forms of the protein. In embodiments, “PI4KA” refers to PI4KIIIβ. In embodiments, “PI4KA” or “PI4K-ALPHA” refers to the protein associated with Entrez Gene 5297, UniProt P42356, and/or RefSeq (protein) NP_477352. In embodiments, the reference numbers immediately above refer to the protein, and associated nucleic acids, known as of the date of filing of this application. In embodiments, “PI4KA” or “PI4K-ALPHA” refers to the wildtype human protein. In embodiments, “PI4KA” or “PI4K-ALPHA” refers to the wildtype human nucleic acid. In embodiments, the PI4KA is a mutant PI4KA protein. In embodiments, the mutant PI4KA is associated with a disease that is not associated with wildtype PI4KA. In embodiments, the PI4KA includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype PI4KA. In embodiments, the PI4KA protein has the protein sequence corresponding to RefSeq NP_477352.3. In embodiments, the PI4KA protein has the protein sequence corresponding to RefSeq NM_058004.3. In embodiments, the PI4KA has the following amino acid sequence:

(SEQ ID NO: 7)
MAAAPARGGGGGGGGGGGCSGSGSSASRGFYFNTVLSLARSLAVQRPASLEKVQKLLCMCP
VDFHGIFQLDERRRDAVIALGIFLIESDLQHKDCVVPYLLRLLKGLPKVYWVEESTARKGRG
ALPVAESFSFCLVTLLSDVAYRDPSLRDEILEVLLQVLHVLLGMCQALEIQDKEYLCKYAIPC
LIGISRAFGRYSNMEESLLSKLFPKIPPHSLRVLEELEGVRRRSFNDFRSILPSNLLTVCQEGTL
KRKTSSVSSISQVSPERGMPPPSSPGGSAFHYFEASCLPDGTALEPEYYFSTISSSFSVSPLFNGV
TYKEFNIPLEMLRELLNLVKKIVEEAVLKSLDAIVASVMEANPSADLYYTSFSDPLYLTMFKM
LRDTLYYMKDLPTSFVKEIHDFVLEQFNTSQGELQKILHDADRIHNELSPLKLRCQANAACV
DLMVWAVKDEQGAENLCIKLSEKLQSKTSSKVIIAHLPLLICCLQGLGRLCERFPVVVHSVTP
SLRDFLVIPSPVLVKLYKYHSQYHTVAGNDIKISVTNEHSESTLNVMSGKKSQPSMYEQLRDI
AIDNICRCLKAGLTVDPVIVEAFLASLSNRLYISQESDKDAHLIPDHTIRALGHIAVALRDTPK
VMEPILQILQQKFCQPPSPLDVLIIDQLGCLVITGNQYIYQEVWNLFQQISVKASSVVYSATKD
YKDHGYRHCSLAVINALANIAANIQDEHLVDELLMNLLELFVQLGLEGKRASERASEKGPAL
KASSSAGNLGVLIPVIAVLTRRLPPIKEAKPRLQKLFRDFWLYSVLMGFAVEGSGLWPEEWY
EGVCEIATKSPLLTFPSKEPLRSVLQYNSAMKNDTVTPAELSELRSTIINLLDPPPEVSALINKL
DFAMSTYLLSVYRLEYMRVLRSTDPDRFQVMFCYFEDKAIQKDKSGMMQCVIAVADKVFD
AFLNMMADKAKTKENEEELERHAQFLLVNFNHIHKRIRRVADKYLSGLVDKFPHLLWSGTV
LKTMLDILQTLSLSLSADIHKDQPYYDIPDAPYRITVPDTYEARESIVKDFAARCGMILQEAM
KWAPTVTKSHLQEYLNKHQNWVSGLSQHTGLAMATESILHFAGYNKQNTTLGATQLSERPA
CVKKDYSNFMASLNLRNRYAGEVYGMIRFSGTTGQMSDLNKMMVQDLHSALDRSHPQHYT
QAMFKLTAMLISSKDCDPQLLHHLCWGPLRMFNEHGMETALACWEWLLAGKDGVEVPFM
REMAGAWHMTVEQKFGLFSAEIKEADPLAASEASQPKPCPPEVTPHYIWIDFLVQRFEIAKYC
SSDQVEIFSSLLQRSMSLNIGGAKGSMNRHVAAIGPRFKLLTLGLSLLHADVVPNATIRNVLR
EKIYSTAFDYFSCPPKFPTQGEKRLREDISIMIKFWTAMFSDKKYLTASQLVPPDNQDTRSNLD
ITVGSRQQATQGWINTYPLSSGMSTISKKSGMSKKTNRGSQLHKYYMKRRTLLLSLLATEIER
LITWYNPLSAPELELDQAGENSVANWRSKYISLSEKQWKDNVNLAWSISPYLAVQLPARFKN
TEAIGNEVTRLVRLDPGAVSDVPEAIKFLVTWHTIDADAPELSHVLCWAPTDPPTGLSYFSSM
YPPHPLTAQYGVKVLRSFPPDAILFYIPQIVQALRYDKMGYVREYILWAASKSQLLAHQFIWN
MKTNIYLDEEGHQKDPDIGDLLDQLVEEITGSLSGPAKDFYQREFDFFNKITNVSAIIKPYPKG
DERKKACLSALSEVKVQPGCYLPSNPEAIVLDIDYKSGTPMQSAAKAPYLAKFKVKRCGVSE
LEKEGLRCRSDSEDECSTQEADGQKISWQAAIFKVGDDCRQDMLALQIIDLFKNIFQLVGLDL
FVFPYRVVATAPGCGVIECIPDCTSRDQLGRQTDFGMYDYFTRQYGDESTLAFQQARYNFIRS
MAAYSLLLFLLQIKDRHNGNIMLDKKGHIIHIDFGFMFESSPGGNLGWEPDIKLTDEMVMIM
GGKMEATPFKWFMEMCVRGYLAVRPYMDAVVSLVTLMLDTGLPCFRGQTIKLLKHRFSPN
MTEREAANFIMKVIQSCFLSNRSRTYDMIQYYQNDIPY.

The term “PIP5K” or “PI4P5K” or “PI5K” refers to the protein “Phosphatidylinositol 4-phosphate 5-kinase”. In embodiments, “PIP5K” or “PI4P5K” or “PI5K” refers to the human protein. Included in the term “PIP5K” or “PI4P5K” or “PI5K” are the wildtype and mutant forms of the protein. In embodiments, “PIP5K” refers to “PIP5K1A”. In embodiments, “PIP5K” or “PI4P5K” or “PI5K” refers to the protein associated with UniProt Q99755, and/or RefSeq (protein) NP_001129110. In embodiments, the reference numbers immediately above refer to the protein, and associated nucleic acids, known as of the date of filing of this application. In embodiments, “PIP5K” or “PI4P5K” or “PI5K” refers to the wildtype human protein. In embodiments, “PIP5K” or “PI4P5K” or “PI5K” refers to the wildtype human nucleic acid. In embodiments, the PIP5K is a mutant PIP5K protein. In embodiments, the mutant PIP5K is associated with a disease that is not associated with wildtype PIP5K. In embodiments, the PIP5K includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype PIP5K. In embodiments, the PIP5K protein has the protein sequence corresponding to RefSeq NP_001129110.1. In embodiments, the PIP5K protein has the protein sequence corresponding to RefSeq NM_001135638.2. In embodiments, the PIP5K has the following amino acid sequence:

(SEQ ID NO: 8)
MASASSGPSSSVGFSSFDPAVPSCTLSSAASGIKRPMASEVLEARQDSYIS
LVPYASGMPIKKIGHRSVDSSGETTYKKTTSSALKGAIQLGITHTVGSLST
KPERDVLMQDFYVVESIFFPSEGSNLTPAHHYNDFRFKTYAPVAFRYFREL
FGIRPDDYLYSLCSEPLIELCSSGASGSLFYVSSDDEFIIKTVQHKEAEFL
QKLLPGYYMNLNQNPRTLLPKFYGLYCVQAGGKNIRIVVMNNLLPRSVKMH
IKYDLKGSTYKRRASQKEREKPLPTFKDLDFLQDIPDGLFLDADMYNALCK
TLQRDCLVLQSFKIMDYSLLMSIHNIDHAQREPLSSETQYSVDTRRPAPQK
ALYSTAMESIQGEARRGGTMETDDHMGGIPARNSKGERLLLYIGIIDILQS
YRFVKKLEHSWKALVHDGDTVSVHRPGFYAERFQRFMCNTVFKKIPLKPSP
SKKFRSGSSFSRRAGSSGNSCITYQPSVSGEHKAQVTTKAEVEPGVHLGRP
DVLPQTPPLEEISEGSPIPDPSFSPLVGETLQMLTTSTTLEKLEVAESEFT
H.

The term “SETD3” refers to the protein “SET domain containing 3 protein”. In embodiments, the term “SETD3” refers to the protein “Su(var)3-9, Enhancer of Zeste, Trithorax domain containing Histone-lysine N-methyltransferase”. In embodiments, “SETD3” refers to the human protein. Included in the term “SETD3” are the wildtype and mutant forms of the protein. In embodiments, “SETD3” refers to the protein associated with Entrez Gene 84193, UniProt Q86TU7, and/or RefSeq (protein) NP_115609. In embodiments, the reference numbers immediately above refer to the protein, and associated nucleic acids, known as of the date of filing of this application. In embodiments, “SETD3” refers to the wildtype human protein. In embodiments, “SETD3” refers to the wildtype human nucleic acid. In embodiments, the SETD3 is a mutant SETD3 protein. In embodiments, the mutant SETD3 is associated with a disease that is not associated with wildtype SETD3. In embodiments, the SETD3 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype SETD3. In embodiments, the SETD3 protein has the protein sequence corresponding to RefSeq NP_115609.2. In embodiments, the SETD3 protein has the protein sequence corresponding to RefSeq NM_032233.3. In embodiments, the SETD3 has the following amino acid sequence:

(SEQ ID NO: 9)
MGKKSRVKTQKSGTGATATVSPKEILNLTSELLQKCSSPAPGPGKEWEEYV
QIRTLVEKIRKKQKGLSVTFDGKREDYFPDLMKWASENGASVEGFEMVNFK
EEGFGLRATRDIKAEELFLWVPRKLLMTVESAKNSVLGPLYSQDRILQAMG
NIALAFHLLCERASPNSFWQPYIQTLPSEYDTPLYFEEDEVRYLQSTQAIH
DVFSQYKNTARQYAYFYKVIQTHPHANKLPLKDSFTYEDYRWAVSSVMTRQ
NQIPTEDGSRVTLALIPLWDMCNHTNGLITTGYNLEDDRCECVALQDFRAG
EQIYIFYGTRSNAEFVIHSGFFFDNNSHDRVKIKLGVSKSDRLYAMKAEVL
ARAGIPTSSVFALHFTEPPISAQLLAFLRVFCMTEEELKEHLLGDSAIDRI
FTLGNSEFPVSWDNEVKLWTFLEDRASLLLKTYKTTIEEDKSVLKNHDLSV
RAKMAIKLRLGEKEILEKAVKSAAVNREYYRQQMEEKAPLPKYEESNLGLL
ESSVGDSRLPLVLRNLEEEAGVQDALNIREAISKAKATENGLVNGENSIPN
GTRSENESLNQESKRAVEDAKGSSSDSTAGVKE.

The term “TRRAP” refers to the protein “Transformation/transcription domain-associated protein”. In embodiments, “TRRAP” refers to the human protein. Included in the term “TRRAP” are the wildtype and mutant forms of the protein. In embodiments, “TRRAP” refers to the protein associated with Entrez Gene 8295, UniProt Q9Y4A5, and/or RefSeq (protein) NP_001231509. In embodiments, the reference numbers immediately above refer to the protein, and associated nucleic acids, known as of the date of filing of this application. In embodiments, “TRRAP” refers to the wildtype human protein. In embodiments, “TRRAP” refers to the wildtype human nucleic acid. In embodiments, the TRRAP is a mutant TRRAP protein. In embodiments, the mutant TRRAP is associated with a disease that is not associated with wildtype TRRAP. In embodiments, the TRRAP includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype TRRAP. In embodiments, the TRRAP protein has the protein sequence corresponding to RefSeq NP_001231509.1. In embodiments, the TRRAP protein has the protein sequence corresponding to RefSeq NM_001244580.1. In embodiments, the TRRAP has the following amino acid sequence:

(SEQ ID NO: 10)
MAFVATQGATVVDQTTLMKKYLQFVAALTDVNTPDETKLKMMQEVSENFENVTSSPQYST
FLEHIIPRFLTFLQDGEVQFLQEKPAQQLRKLVLEIIHRIPTNEHLRPHTKNVLSVMFRFLETEN
EENVLICLRIIIELHKQFRPPITQEIHHFLDFVKQIYKELPKVVNRYFENPQVIPENTVPPPEMVG
MITTIAVKVNPEREDSETRTHSIIPRGSLSLKVLAELPIIVVLMYQLYKLNIHNVVAEFVPLIMN
TIAIQVSAQARQHKLYNKELYADFIAAQIKTLSFLAYIIRIYQELVTKYSQQMVKGMLQLLSN
CPAETAHLRKELLIAAKHILTTELRNQFIPCMDKLFDESILIGSGYTARETLRPLAYSTLADLV
HHVRQHLPLSDLSLAVQLFAKNIDDESLPSSIQTMSCKLLLNLVDCIRSKSEQESGNGRDVLM
RMLEVFVLKFHTIARYQLSAIFKKCKPQSELGAVEAALPGVPTAPAAPGPAPSPAPVPAPPPPP
PPPPPATPVTPAPVPPFEKQGEKDKEDKQTFQVTDCRSLVKTLVCGVKTITWGITSCKAPGEA
QFIPNKQLQPKETQIYIKLVKYAMQALDIYQVQIAGNGQTYIRVANCQTVRMKEEKEVLEHF
AGVFTMMNPLTFKEIFQTTVPYMVERISKNYALQIVANSFLANPTTSALFATILVEYLLDRLPE
MGSNVELSNLYLKLFKLVFGSVSLFAAENEQMLKPHLHKIVNSSMELAQTAKEPYNYFLLLR
ALFRSIGGGSHDLLYQEFLPLLPNLLQGLNMLQSGLHKQHMKDLFVELCLTVPVRLSSLLPYL
PMLMDPLVSALNGSQTLVSQGLRTLELCVDNLQPDFLYDHIQPVRAELMQALWRTLRNPAD
SISHVAYRVLGKFGGSNRKMLKESQKLHYVVTEVQGPSITVEFSDCKASLQLPMEKAIETAL
DCLKSANTEPYYRRQAWEVIKCFLVAMMSLEDNKHALYQLLAHPNFTEKTIPNVIISHRYKA
QDTPARKTFEQALTGAFMSAVIKDLRPSALPFVASLIRHYTMVAVAQQCGPFLLPCYQVGSQ
PSTAMFHSEENGSKGMDPLVLIDAIAICMAYEEKELCKIGEVALAVIFDVASIILGSKERACQL
PLFSYIVERLCACCYEQAWYAKLGGVVSIKFLMERLPLTWVLQNQQTFLKALLFVMMDLTG
EVSNGAVAMAKTTLEQLLMRCATPLKDEERAEEIVAAQEKSFHHVTHDLVREVTSPNSTVR
KQAMHSLQVLAQVTGKSVTVIMEPHKEVLQDMVPPKKHLLRHQPANAQIGLMEGNTFCTTL
QPRLFTMDLNVVEHKVFYTELLNLCEAEDSALTKLPCYKSLPSLVPLRIAALNALAACNYLP
QSREKIIAALFKALNSTNSELQEAGEACMRKFLEGATIEVDQIHTHMRPLLMMLGDYRSLTL
NVVNRLTSVTRLFPNSFNDKFCDQMMQHLRKWMEVVVITHKGGQRSDGNESISECGRCPLS
PFCQFEEMKICSAIINLFHLIPAAPQTLVKPLLEVVMKTERAMLIEAGSPFREPLIKFLTRHPSQ
TVELFMMEATLNDPQWSRMFMSFLKHKDARPLRDVLAANPNRFITLLLPGGAQTAVRPGSP
STSTMRLDLQFQAIKIISIIVKNDDSWLASQHSLVSQLRRVWVSENFQERHRKENMAATNWK
EPKLLAYCLLNYCKRNYGDIELLFQLLRAFTGRFLCNMTFLKEYMEEEIPKNYSIAQKRALFF
RFVDFNDPNFGDELKAKVLQHILNPAFLYSFEKGEGEQLLGPPNPEGDNPESITSVFITKVLDP
EKQADMLDSLRIYLLQYATLLVEHAPHHIHDNNKNRNSKLRRLMTFAWPCLLSKACVDPAC
KYSGHLLLAHIIAKFAIHKKIVLQVFHSLLKAHAMEARAIVRQAMAILTPAVPARMEDGHQM
LTHWTRKIIVEEGHTVPQLVHILHLIVQHFKVYYPVRHHLVQHMVSAMQRLGFTPSVTIEQR
RLAVDLSEVVIKWELQRIKDQQPDSDMDPNSSGEGVNSVSSSIKRGLSVDSAQEVKRFRTAT
GAISAVFGRSQSLPGADSLLAKPIDKQHTDTVVNFLIRVACQVNDNTNTAGSPGEVLSRRCV
NLLKTALRPDMWPKSELKLQWFDKLLMTVEQPNQVNYGNICTGLEVLSFLLTVLQSPAILSS
FKPLQRGIAACMTCGNTKVLRAVHSLLSRLMSIFPTEPSTSSVASKYEELECLYAAVGKVIYE
GLTNYEKATNANPSQLFGTLMILKSACSNNPSYIDRLISVFMRSLQKMVREHLNPQAASGSTE
ATSGTSELVMLSLELVKTRLAVMSMEMRKNFIQAILTSLIEKSPDAKILRAVVKIVEEWVKNN
SPMAANQTPTLREKSILLVKMMTYIEKRFPEDLELNAQFLDLVNYVYRDETLSGSELTAKLEP
AFLSGLRCAQPLIRAKFFEVFDNSMKRRVYERLLYVTCSQNWEAMGNHFWIKQCIELLLAVC
EKSTPIGTSCQGAMLPSITNVINLADSHDRAAFAMVTHVKQEPRERENSESKEEDVEIDIELAP
GDQTSTPKTKELSEKDIGNQLHMLTNRHDKFLDTLREVKTGALLSAFVQLCHISTTLAEKTW
VQLFPRLWKILSDRQQHALAGEISPFLCSGSHQVQRDCQPSALNCFVEAMSQCVPPIPIRPCVL
KYLGKTHNLWFRSTLMLEHQAFEKGLSLQIKPKQTTEFYEQESITPPQQEILDSLAELYSLLQE
EDMWAGLWQKRCKYSETATAIAYEQHGFFEQAQESYEKAMDKAKKEHERSNASPAIFPEY
QLWEDHWIRCSKELNQWEALTEYGQSKGHINPYLVLECAWRVSNWTAMKEALVQVEVSCP
KEMAWKVNMYRGYLAICHPEEQQLSFIERLVEMASSLAIREWRRLPHVVSHVHTPLLQAAQ
QIIELQEAAQINAGLQPTNLGRNNSLHDMKTVVKTWRNRLPIVSDDLSHWSSIFMWRQHHY
QGKPTWSGMHSSSIVTAYENSSQHDPSSNNAMLGVHASASAIIQYGKIARKQGLVNVALDIL
SRIHTIPTVPIVDCFQKIRQQVKCYLQLAGVMGKNECMQGLEVIESTNLKYFTKEMTAEFYAL
KGMFLAQINKSEEANKAFSAAVQMHDVLVKAWAMWGDYLENIFVKERQLHLGVSAITCYL
HACRHQNESKSRKYLAKVLWLLSFDDDKNTLADAVDKYCIGVPPIQWLAWIPQLLTCLVGS
EGKLLLNLISQVGRVYPQAVYFPIRTLYLTLKIEQRERYKSDPGPIRATAPMWRCSRIMHMQR
ELHPTLLSSLEGIVDQMVWFRENWHEEVLRQLQQGLAKCYSVAFEKSGAVSDAKITPHTLNF
VKKLVSTFGVGLENVSNVSTMFSSAASESLARRAQATAQDPVFQKLKGQFTTDFDFSVPGSM
KLHNLISKLKKWIKILEAKTKQLPKFFLIEEKCRFLSNFSAQTAEVEIPGEFLMPKPTHYYIKIA
RFMPRVEIVQKHNTAARRLYIRGHNGKIYPYLVMNDACLTESRREERVLQLLRLLNPCLEKR
KETTKRHLFFTVPRVVAVSPQMRLVEDNPSSLSLVEIYKQRCAKKGIEHDNPISRYYDRLATV
QARGTQASHQVLRDILKEVQSNMVPRSMLKEWALHTFPNATDYWTFRKMFTIQLALIGFAE
FVLHLNRLNPEMLQIAQDTGKLNVAYFRFDINDATGDLDANRPVPFRLTPNISEFLTTIGVSGP
LTASMIAVARCFAQPNFKVDGILKTVLRDEIIAWHKKTQEDTSSPLSAAGQPENMDSQQLVS
LVQKAVTAIMTRLHNLAQFEGGESKVNTLVAAANSLDNLCRMDPAWHPWL.

The term “MAP4K” or “mitogen-activated protein kinase kinase kinase kinase” refers to the family of serine/threonine kinases involved in cellular signal transduction. In embodiments, MAP4K is MAP4K1 or hematopoietic progenitor kinase 1 (HPK1). In embodiments, MAP4K is MAP4K2 or germinal center kinase (GCK). In embodiments, MAP4K is MAP4K3 or germinal center kinase-like kinase (GLK). In embodiments, MAP4K is MAP4K4 or hepatocyte progenitor kinase-like/germinal center kinase-like kinase (HGK). In embodiments, MAP4K is MAP4K5 or kinase homologous to SPS1/STE20 (KHS). In embodiments, MAP4K is MAP4K6 or misshapen-like kinase 1 (MINK).

The term “hepatocyte progenitor kinase-like/germinal center kinase-like kinase” or “HGK” or “MAP4K4” is encoded by the MAP4K4 gene. The term “HGK” may refer to the nucleotide sequence or protein sequence of human HGK (e.g., Entrez 9448, Uniprot 095819, RefSeq NM_00124559.1, RefSeq NM_001242560, RefSeq NM_004834.4, RefSeq NM_145686.3, RefSeq NM_145687.3, RefSeq NP_001229488.1, RefSeq NP_001229489, RefSeq NP_004825.3, RefSeq NP_663719.2, or RefSeq NP_663720.1). The term “HGK” includes both the wild-type form of the nucleotide sequences or proteins as well as any mutants thereof. In some embodiments, “HGK” is wild-type HGK. In some embodiments, “HGK” is one or more mutant forms. The term “HGK” XYZ refers to a nucleotide sequence or protein of a mutant HGK wherein the Y numbered amino acid of HGK that normally has an X amino acid in the wildtype, instead has a Z amino acid in the mutant. In embodiments, an HGK is the human HGK. In embodiments, the HGK has the following amino acid sequence.

(SEQ ID NO: 11)
MANDSPAKSLVDIDLSSLRDPAGIFELVEVVGNGTYGQVYKGRHVKTGQLAAIKVMDVTED
EEEEIKLEINMLKKYSHHRNIATYYGAFIKKSPPGHDDQLWLVMEFCGAGSITDLVKNTKGN
TLKEDWIAYISREILRGLAHLHIHHVIHRDIKGQNVLLTENAEVKLVDFGVSAQLDRTVGRRN
TFIGTPYWMAPEVIACDENPDATYDYRSDLWSCGITAIEMAEGAPPLCDMHPMRALFLIPRNP
PPRLKSKKWSKKFFSFIEGCLVKNYMQRPSTEQLLKHPFIRDQPNERQVRIQLKDHIDRTRKK
RGEKDETEYEYSGSEEEEEEVPEQEGEPSSIVNVPGESTLRRDFLRLQQENKERSEALRRQQLL
QEQQLREQEEYKRQLLAERQKRIEQQKEQRRRLEEQQRREREARRQQEREQRRREQEEKRR
LEELERRRKEEEERRRAEEEKRRVEREQEYIRRQLEEEQRHLEVLQQQLLQEQAMLLECRWR
EMEEHRQAERLQRQLQQEQAYLLSLQHDHRRPHPQHSQQPPPPQQERSKPSFHAPEPKAHYE
PADRAREVEDRFRKTNHSSPEAQSKQTGRVLEPPVPSRSESFSNGNSESVHPALQRPAEPQVP
VRTTSRSPVLSRRDSPLQGSGQQNSQAGQRNSTSIEPRLLWERVEKLVPRPGSGSSSGSSNSGS
QPGSHPGSQSGSGERFRVRSSSKSEGSPSQRLENAVKKPEDKKEVFRPLKPADLTALAKELRA
VEDVRPPHKVTDYSSSSEESGTTDEEDDDVEQEGADESTSGPEDTRAASSLNLSNGETESVKT
MIVHDDVESEPAMTPSKEGTLIVRQTQSASSTLQKHKSSSSFTPFIDPRLLQISPSSGTTVTSVV
GFSCDGMRPEAIRQDPTRKGSVVNVNPTNTRPQSDTPEIRKYKKRFNSEILCAALWGVNLLV
GTESGLMLLDRSGQGKVYPLINRRRFQQMDVLEGLNVLVTISGKKDKLRVYYLSWLRNKIL
HNDPEVEKKQGWTTVGDLEGCVHYKVVKYERIKFLVIALKSSVEVYAWAPKPYHKFMAFK
SFGELVHKPLLVDLTVEEGQRLKVIYGSCAGFHAVDVDSGSVYDIYLPTHIQCSIKPHAIIILPN
TDGMELLVCYEDEGVYVNTYGRITKDVVLQWGEMPTSVAYIRSNQTMGWGEKAIEIRSVET
GHLDGVFMHKRAQRLKFLCERNDKVFFASVRSGGSSQVYFMTLGRTSLLSW.

The term “MAP3K” or “mitogen-activated protein kinase kinase kinase” refers to the family of serine/threonine-specific protein kinases. In embodiments, MAP3K is MAP3K12 or dual leucine zipper bearing kinase (DLK).

The term “dual leucine zipper bearing kinase” or “DLK” or “MAP3K12” is encoded by the MAP3K12 gene. The term “DLK” may refer to the nucleotide sequence or protein sequence of human DLK (e.g., Entrez 7786, Uniprot Q12852, RefSeq NM_001193511.1, RefSeq NM_006301.3, RefSeq NP_001180440.1, or RefSeq NP_006292.3). The term “DLK” includes both the wild-type form of the nucleotide sequences or proteins as well as any mutants thereof. In some embodiments, “DLK” is wild-type DLK. In some embodiments, “DLK” is one or more mutant forms. The term “DLK” XYZ refers to a nucleotide sequence or protein of a mutant DLK wherein the Y numbered amino acid of DLK that normally has an X amino acid in the wildtype, instead has a Z amino acid in the mutant. In embodiments, an DLK is the human DLK. In embodiments, the DLK has the following amino acid sequence:

(SEQ ID NO: 12)
MACLHETRTPSPSFGGFVSTLSEASMRKLDPDTSDCTPEKDLTPTHVLQLHEQDAGGPGGAA
GSPESRASRVRADEVRLQCQSGSGFLEGLFGCLRPVWTMIGKAYSTEHKQQQEDLWEVPFEE
ILDLQWVGSGAQGAVFLGRFHGEEVAVKKVRDLKETDIKHLRKLKHPNIITFKGVCTQAPCY
CILMEFCAQGQLYEVLRAGRPVTPSLLVDWSMGIAGGMNYLHLHKIIHRDLKSPNMLITYDD
VVKISDFGTSKELSDKSTKMSFAGTVAWMAPEVIRNEPVSEKVDIWSFGVVLWELLTGEIPY
KDVDSSAIIWGVGSNSLHLPVPSSCPDGFKILLRQCWNSKPRNRPSFRQILLHLDIASADVLST
PQETYFKSQAEWREEVKLHFEKIKSEGTCLHRLEEELVMRRREELRHALDIREHYERKLERA
NNLYMELNALMLQLELKERELLRREQALERRCPGLLKPHPSRGLLHGNTMEKLIKKRNVPQ
KLSPHSKRPDILKTESLLPKLDAALSGVGLPGCPKGPPSPGRSRRGKTRHRKASAKGSCGDLP
GLRTAVPPHEPGGPGSPGGLGGGPSAWEACPPALRGLHHDLLLRKMSSSSPDLLSAALGSRG
RGATGGAGDPGSPPPARGDTPPSEGSAPGSTSPDSPGGAKGEPPPPVGPGEGVGLLGTGREGT
SGRGGSRAGSQHLTPAALLYRAAVTRSQKRGISSEEEEGEVDSEVELTSSQRWPQSLNMRQS
LSTFSSENPSDGEEGTASEPSPSGTPEVGSTNTDERPDERSDDMCSQGSEIPLDPPPSEVIPGPEP
SSLPIPHQELLRERGPPNSEDSDCDSTELDNSNSVDALRPPASLPP.

An “immunophilin blocking agent” is an agent (e.g., compound, small molecule, nucleic acid, or protein) capable of inhibiting or reducing contact between an immunophilin binding compound described herein and an immunophilin wherein the immunophilin blocking agent is deficient in biological activity (e.g., not capable of inhibiting an immune response or T cell activity, reduced or lacking binding to calcineurin) not associated with blocking binding to immunophilin of a separate immunophilin binding compound (e.g., compound described herein).

### II. Compounds

In one aspect is provided a compound having the formula:

A-L¹-R¹.

A is an immunophilin-binding moiety.

L¹ is a bond or a covalent linker.

R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

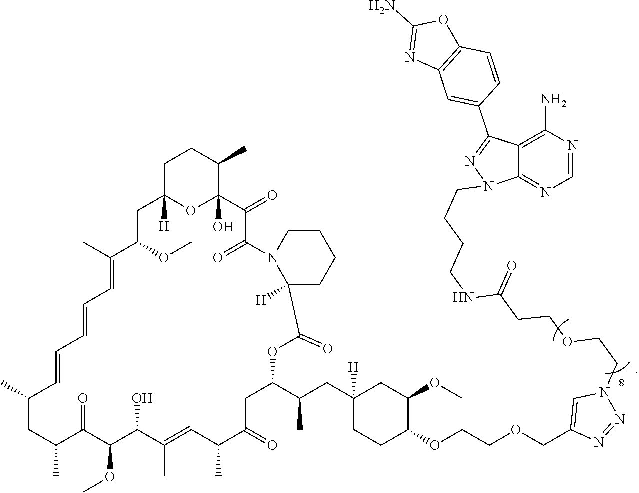

In an aspect is provided a compound having the formula:

A-L¹-R¹; A is an immunophilin-binding moiety; L¹ is a bond or a covalent linker; and R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

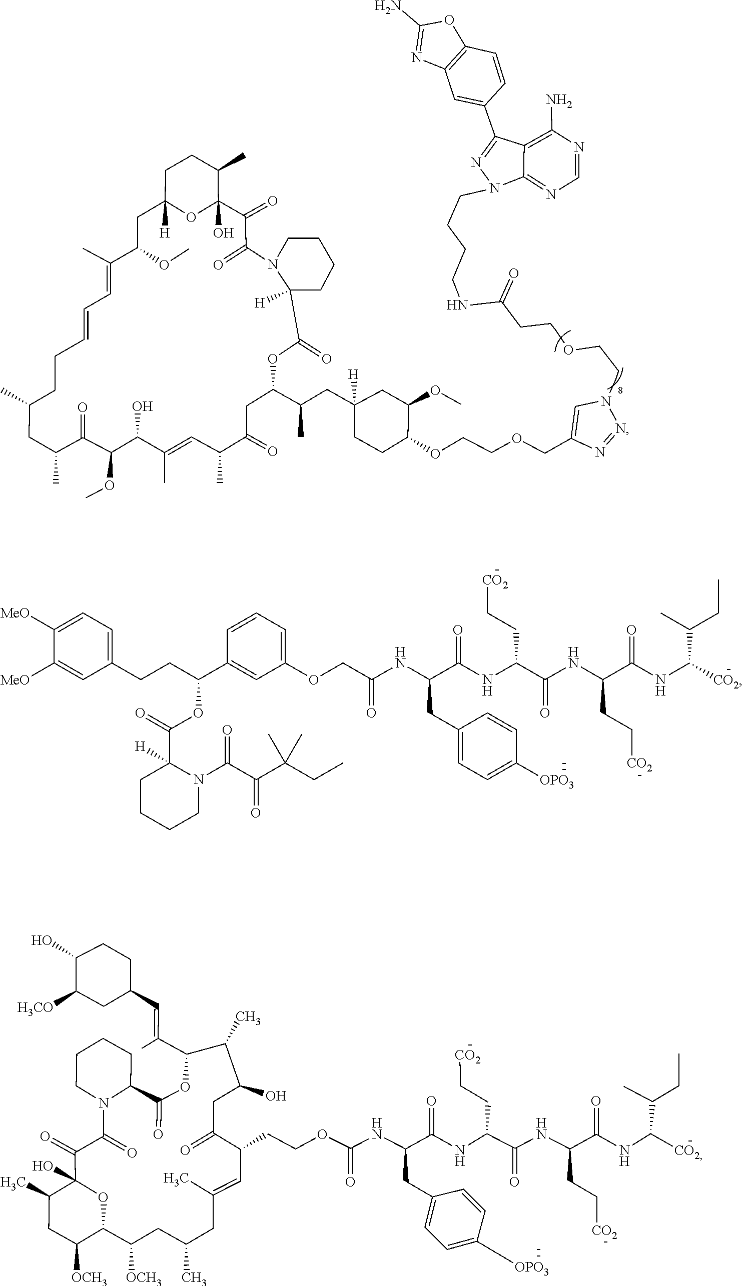

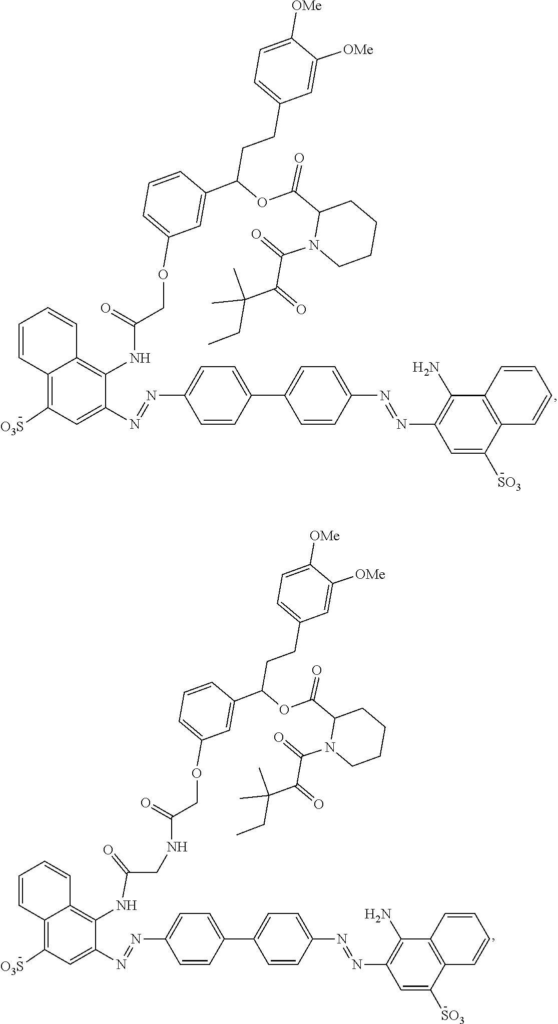

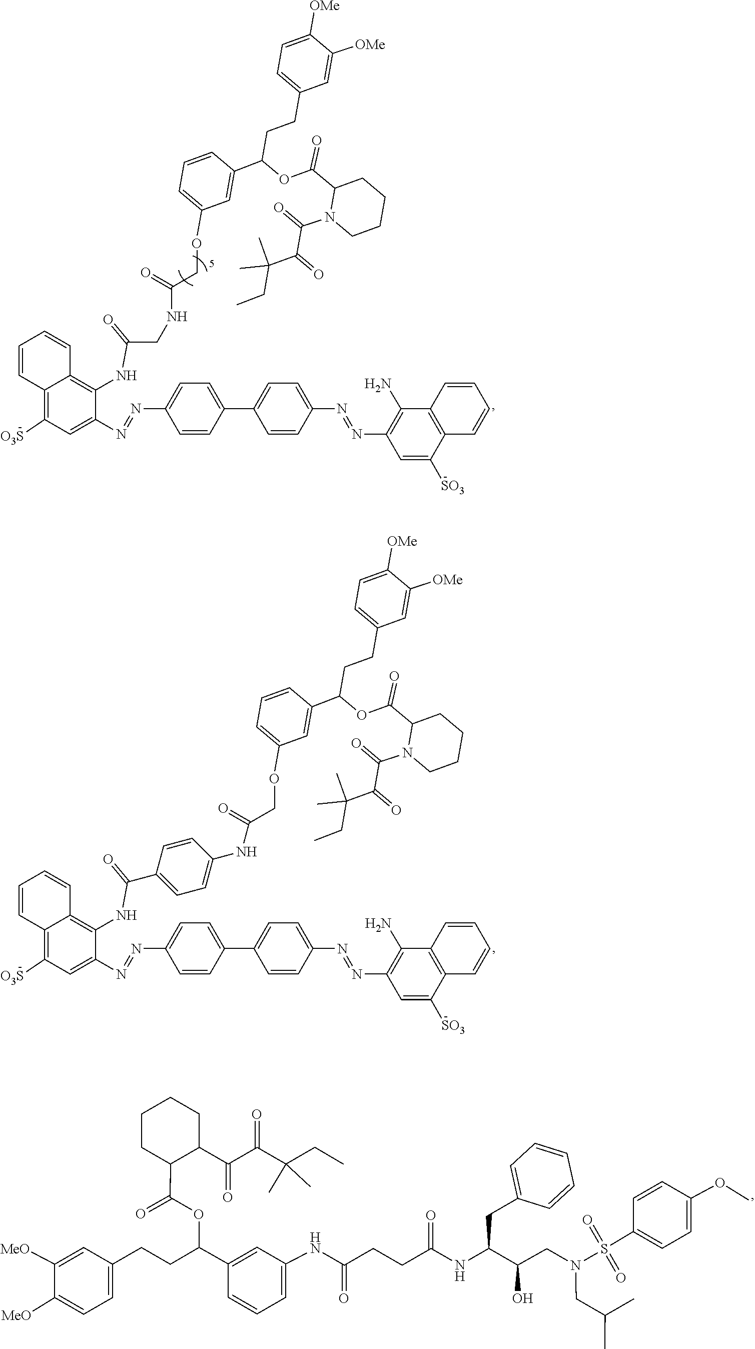

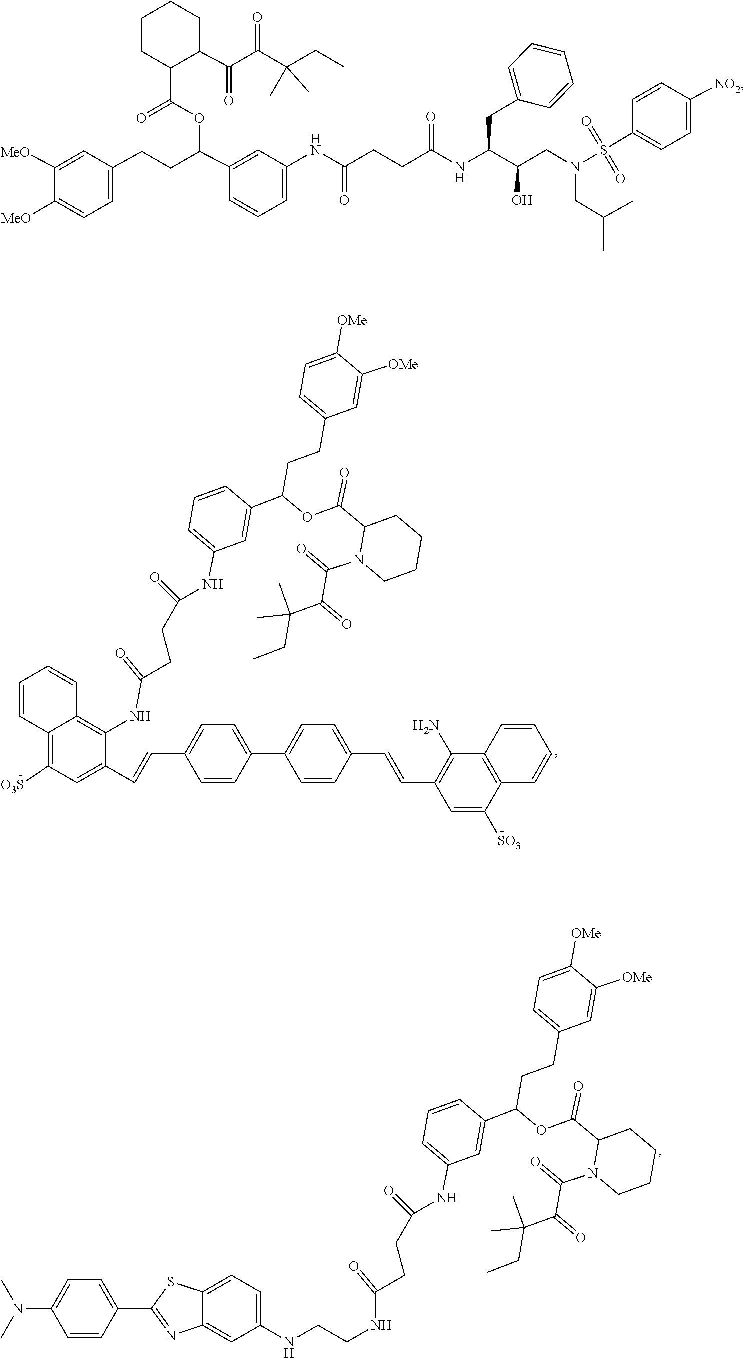

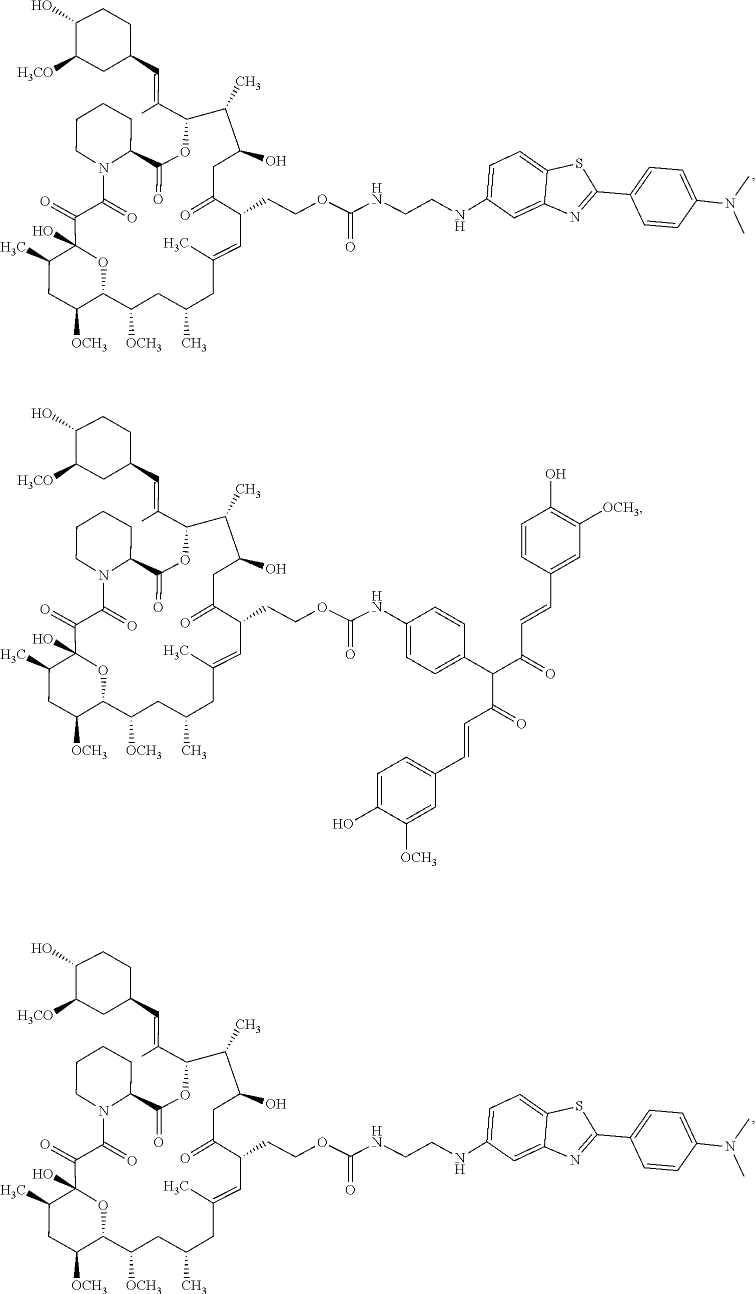

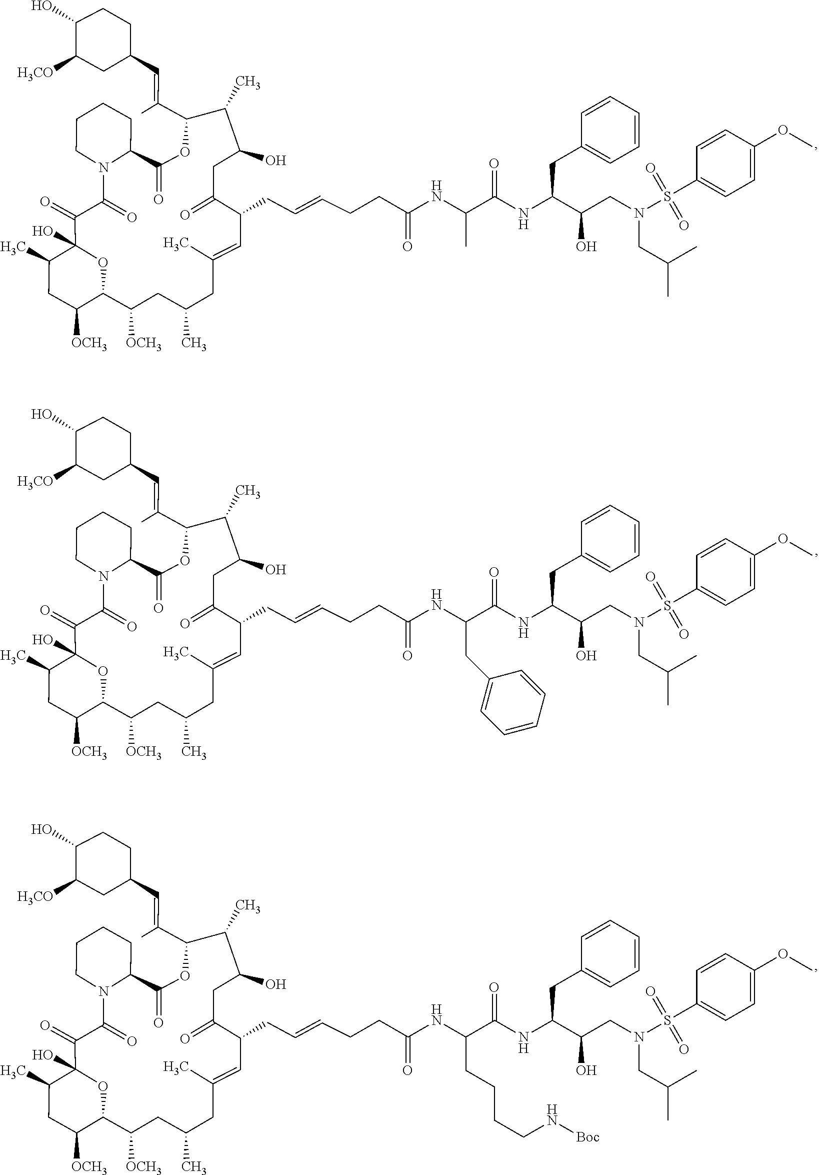

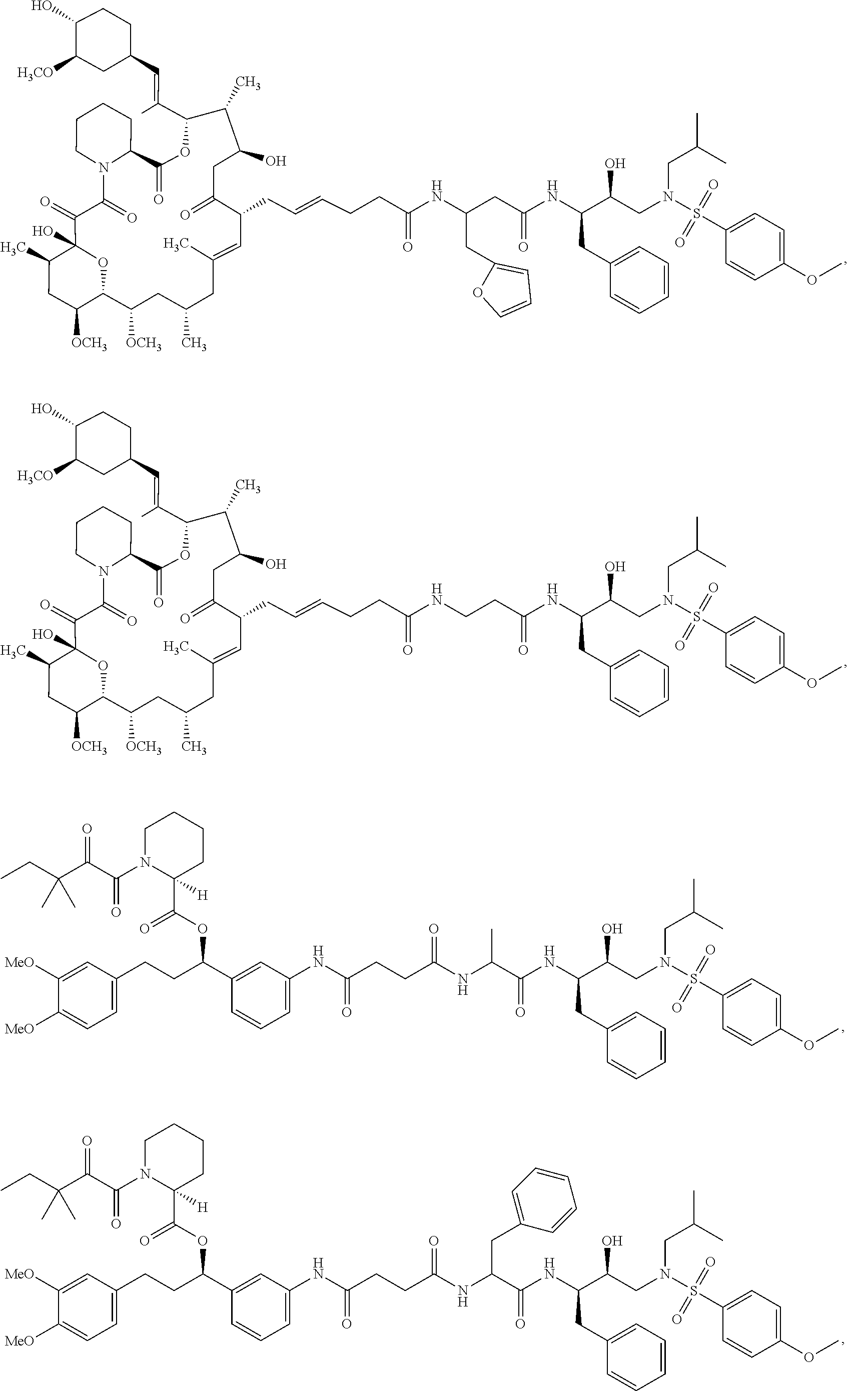

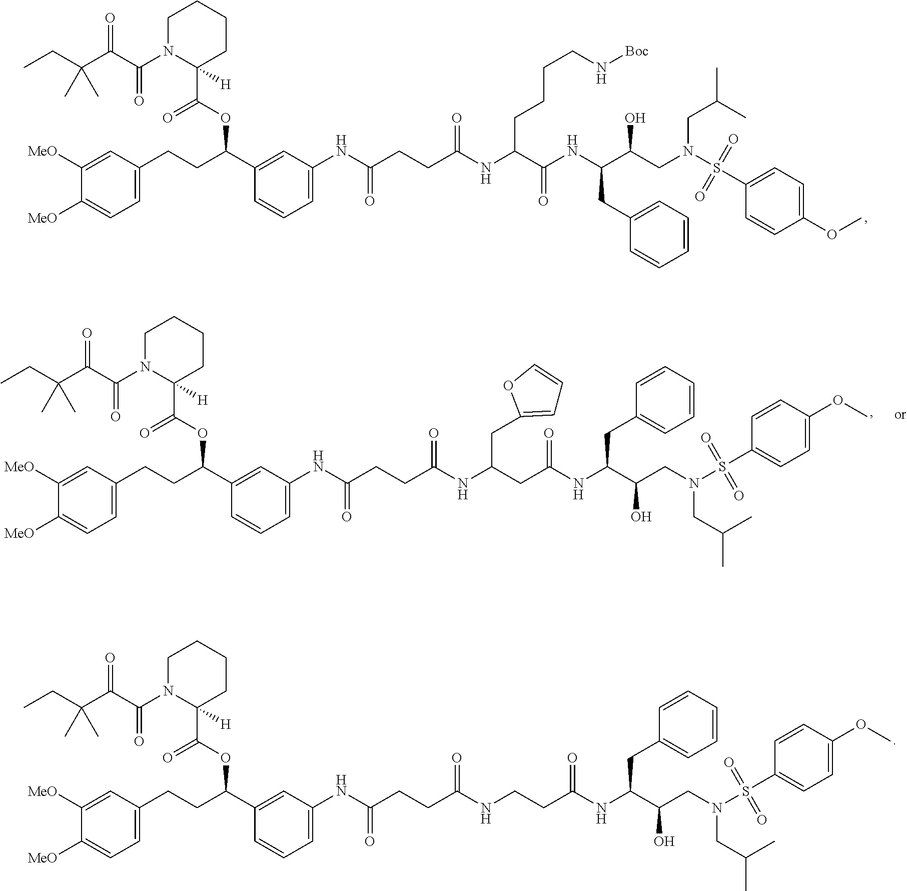

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

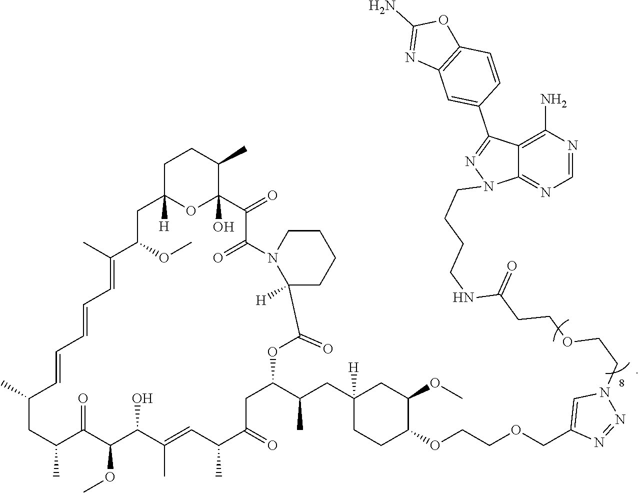

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

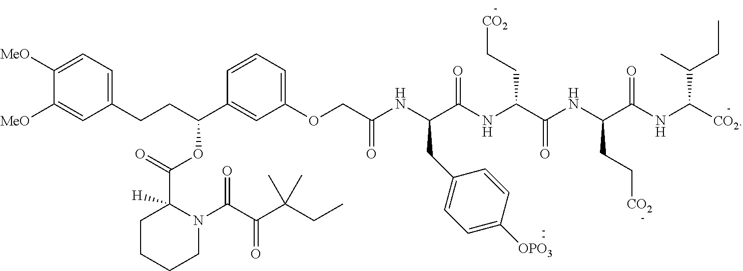

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

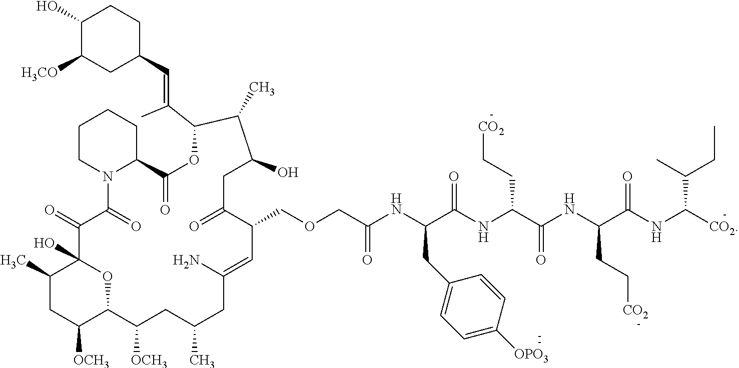

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

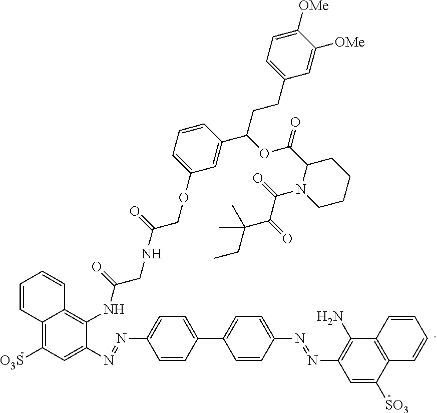

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

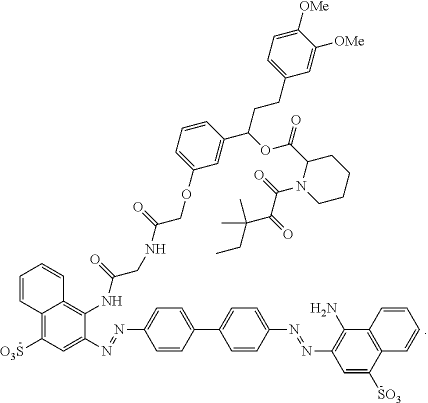

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

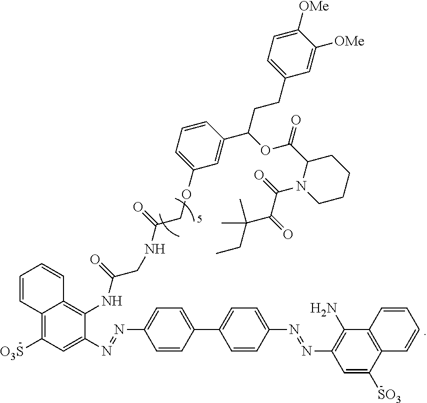

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

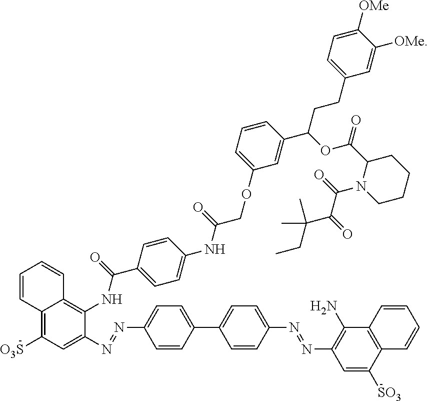

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

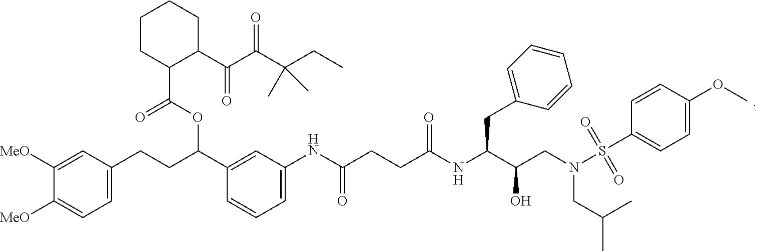 [SMILES: CCC(C)(C)C(=O)C(=O)C1CCCCC1C(=O)OC(CCc1ccc(OC)c(OC)c1)c1cccc(NC(=O)CCC(=O)N[C@@H](Cc2ccccc2)[C@H](O)CN(CC(C)C)S(=O)(=O)c2ccc(OC)cc2)c1]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

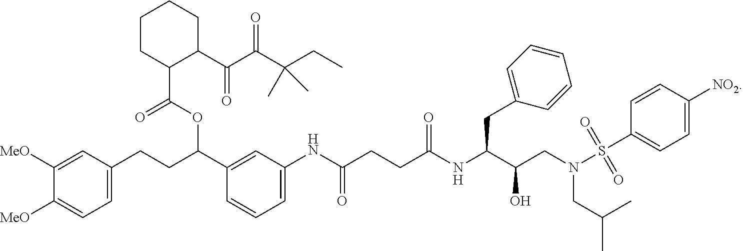 [SMILES: CCC(C)(C)C(=O)C(=O)C1CCCCC1C(=O)OC(CCc1ccc(OC)c(OC)c1)c1cccc(NC(=O)CCC(=O)N[C@@H](Cc2ccccc2)[C@H](O)CN(CC(C)C)S(=O)(=O)c2ccc([N+](=O)[O-])cc2)c1]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

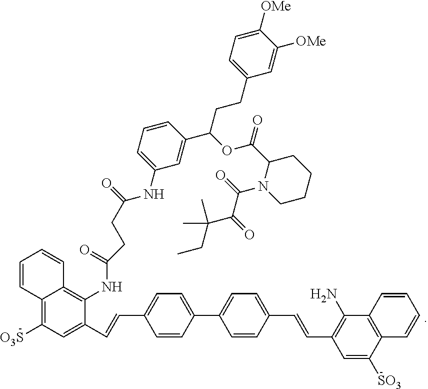

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

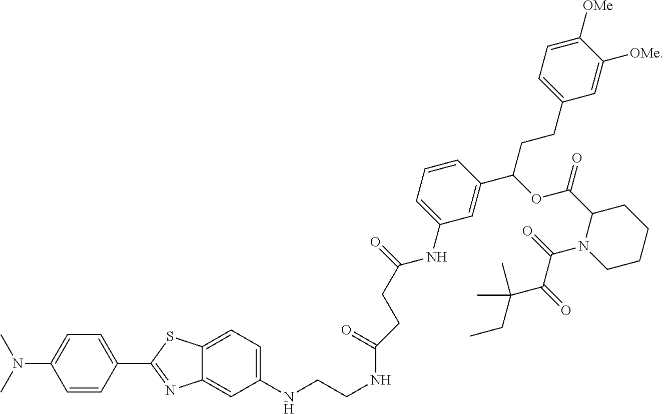 [SMILES: CCC(C)(C)C(=O)C(=O)N1CCCCC1C(=O)OC(CCc1ccc(OC)c(OC)c1)c1cccc(NC(=O)CCC(=O)NCCNc2ccc3sc(-c4ccc(N(C)C)cc4)nc3c2)c1]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

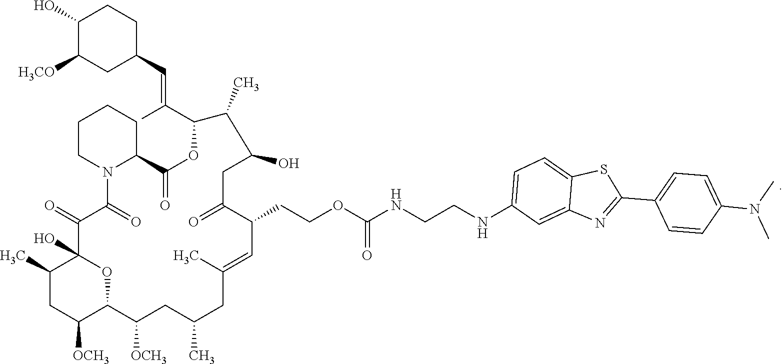

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

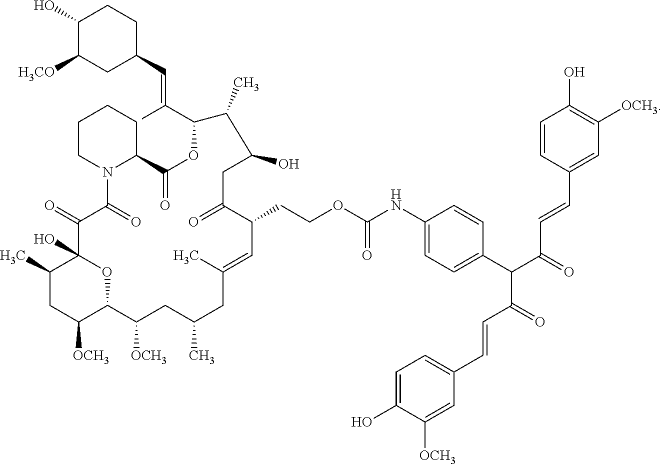 [SMILES: COc1cc(/C=C/C(=O)C(C(=O)/C=C/c2ccc(O)c(OC)c2)c2ccc(NC(=O)OCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/[C@@H]5CC[C@@H](O)[C@H](OC)C5)[C@H](C)[C@@H](O)CC3=O)[C@H](C)C[C@@H]4OC)cc2)ccc1O]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

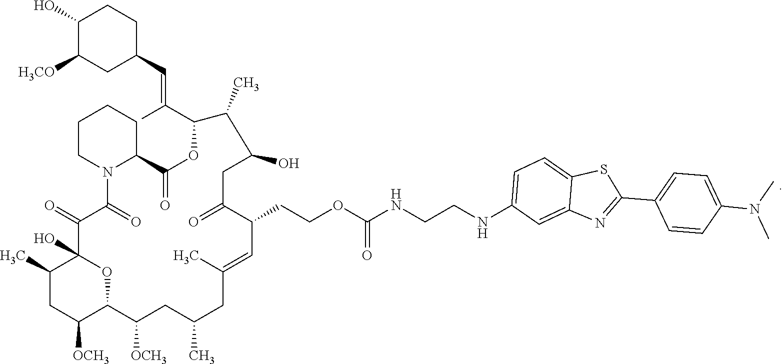

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

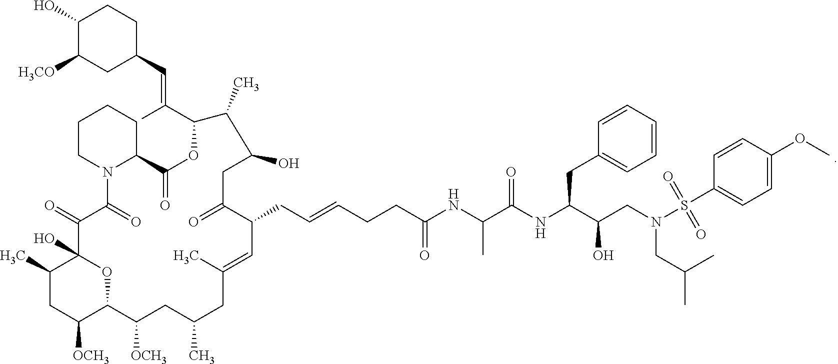

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

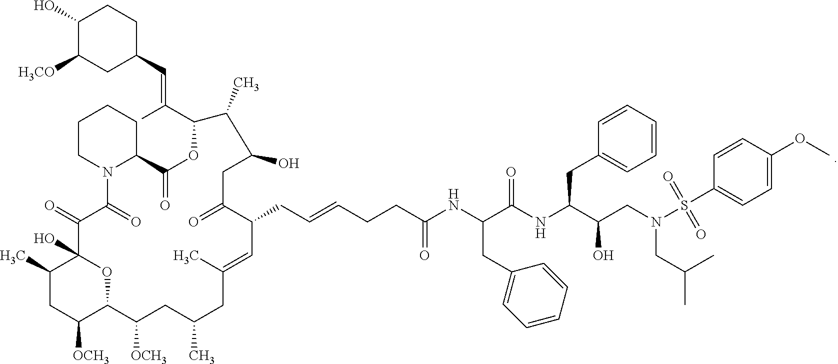

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

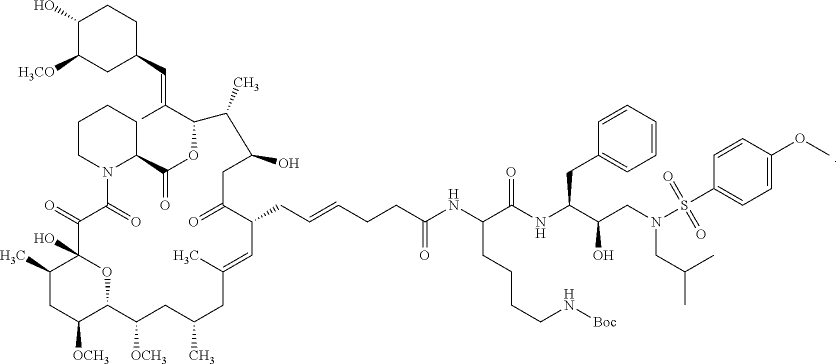

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

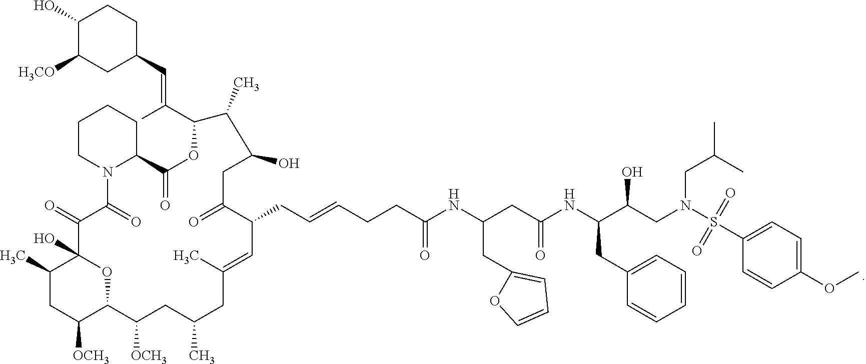

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

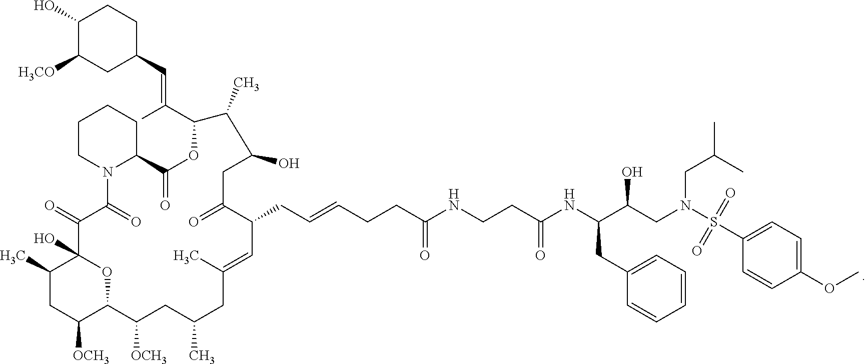

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

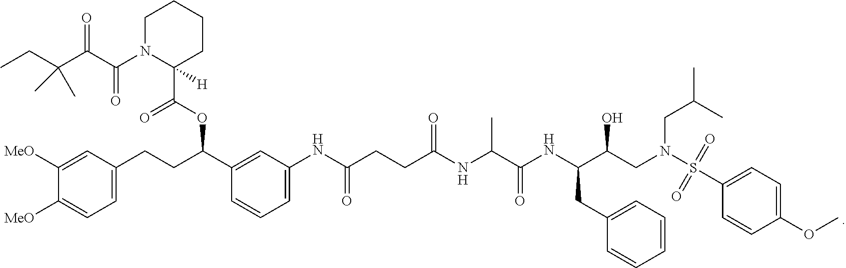 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(C)C(=O)N[C@H](Cc2ccccc2)[C@@H](O)CN(CC(C)C)S(=O)(=O)c2ccc(OC)cc2)c1]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

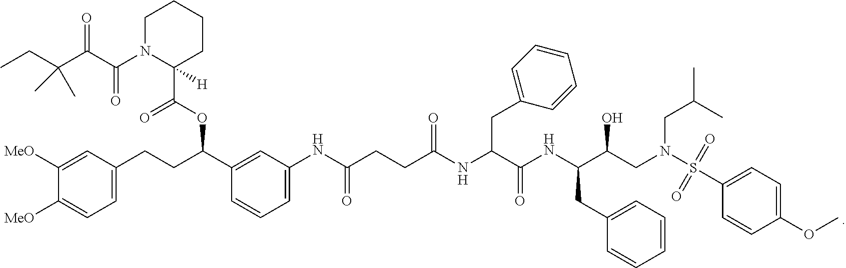 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(Cc2ccccc2)C(=O)N[C@H](Cc2ccccc2)[C@@H](O)CN(CC(C)C)S(=O)(=O)c2ccc(OC)cc2)c1]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

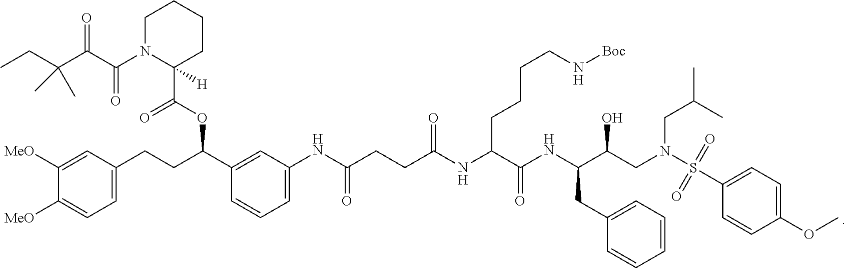 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(CCCCNC(=O)OC(C)(C)C)C(=O)N[C@H](Cc2ccccc2)[C@@H](O)CN(CC(C)C)S(=O)(=O)c2ccc(OC)cc2)c1]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

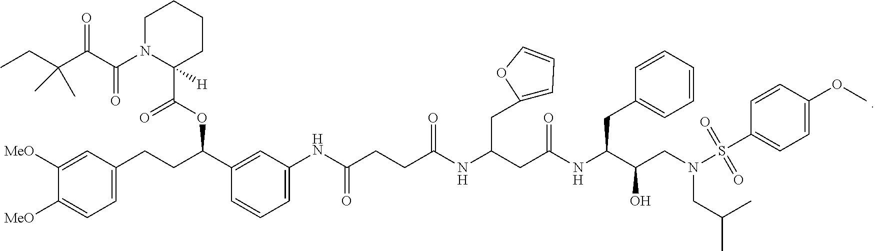 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(CC(=O)N[C@@H](Cc2ccccc2)[C@H](O)CN(CC(C)C)S(=O)(=O)c2ccc(OC)cc2)Cc2ccco2)c1]

In embodiments, R¹ is a monovalent kinase inhibitor, a monovalent pseudokinase inhibitor, a monovalent GTPase inhibitor, a monovalent histone-modifying enzyme inhibitor, or monovalent anti-viral agent; wherein the compound is not

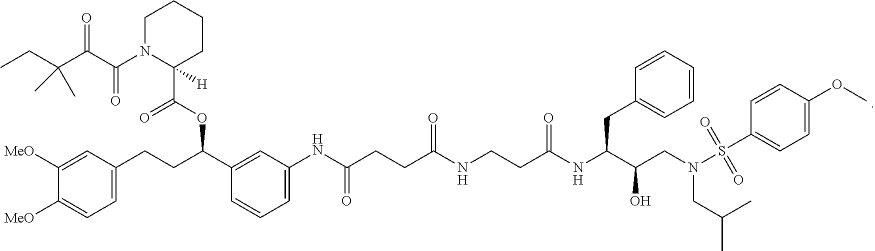 [SMILES: CCC(C)(C)C(=O)C(=O)N1CCCC[C@H]1C(=O)O[C@H](CCc1ccc(OC)c(OC)c1)c1cccc(NC(=O)CCC(=O)NCCC(=O)N[C@@H](Cc2ccccc2)[C@H](O)CN(CC(C)C)S(=O)(=O)c2ccc(OC)cc2)c1]

In embodiments, the immunophilin-binding moiety is a cyclophilin-binding moiety or an FKBP-binding moiety. In embodiments, the immunophilin-binding moiety is

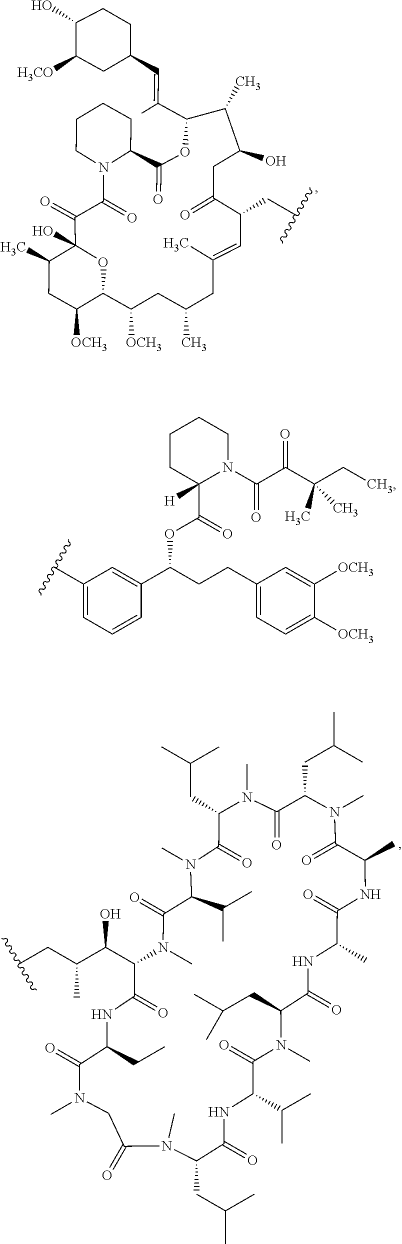

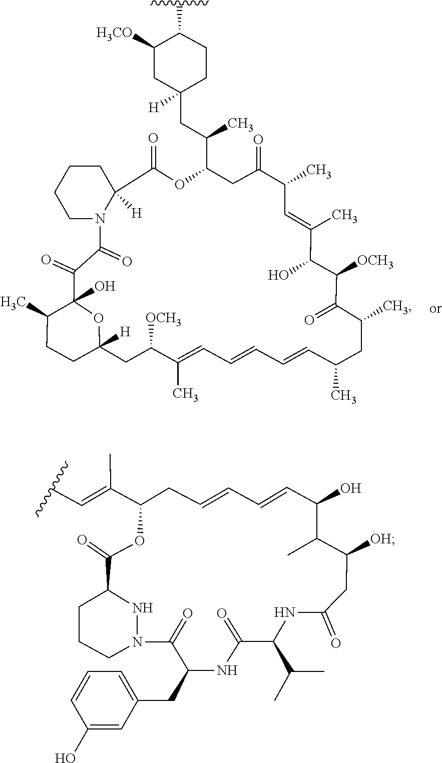 [SMILES: *[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC ; */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

or an analog thereof.

In embodiments, the immunophilin-binding moiety is

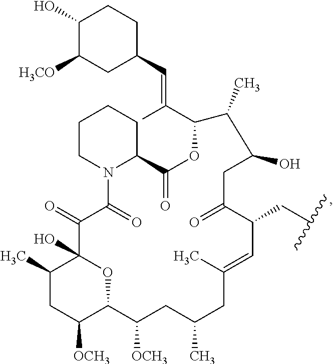 [SMILES: *C[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

or an analog thereof.

In embodiments, the immunophilin-binding moiety is

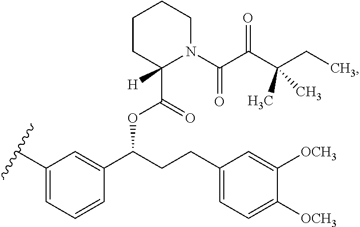

or an analog thereof.

In embodiments, the immunophilin-binding moiety is

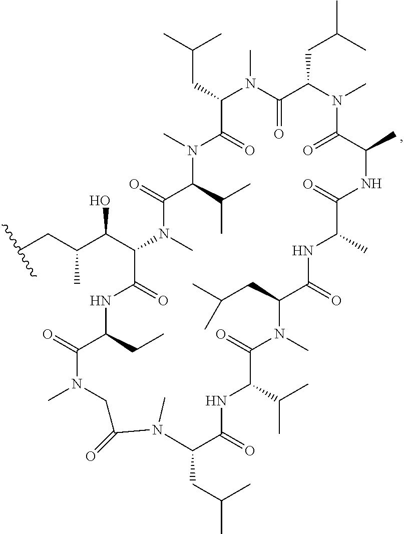 [SMILES: *C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

or an analog thereof.

In embodiments, the immunophilin-binding moiety is

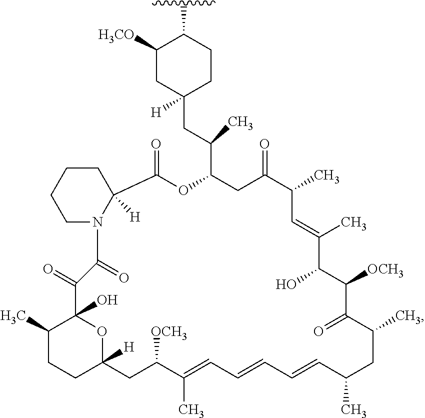 [SMILES: *[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

or an analog thereof.

In embodiments, the immunophilin-binding moiety is or

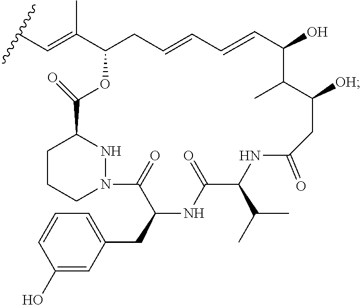 [SMILES: */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

or an analog thereof.

In embodiments, the immunophilin-binding moiety is

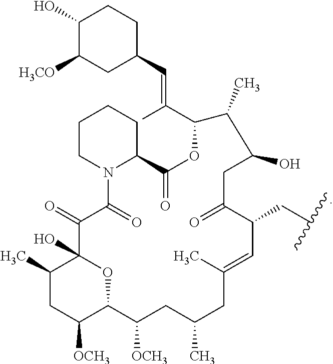 [SMILES: *C[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

In embodiments, the immunophilin-binding moiety is

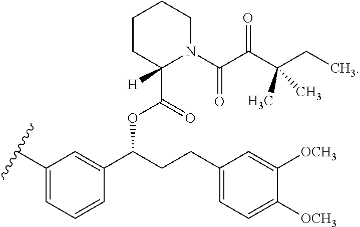

In embodiments, the immunophilin-binding moiety is

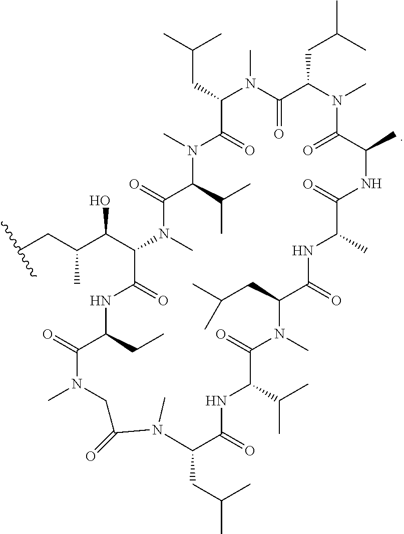 [SMILES: *C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

In embodiments, the immunophilin-binding moiety is

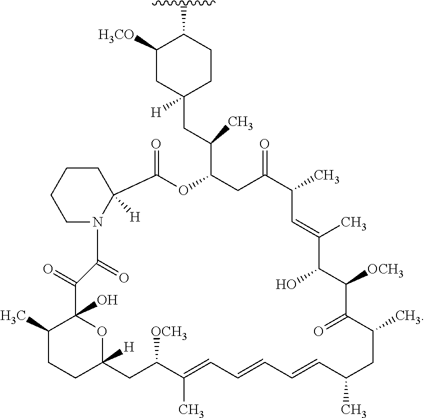 [SMILES: *[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

In embodiments, the immunophilin-binding moiety is

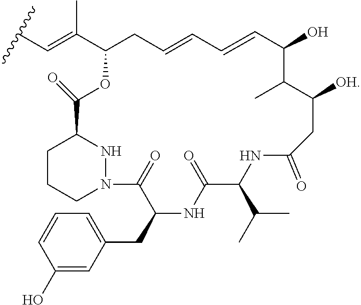 [SMILES: */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

In embodiments, the immunophilin-binding moiety is

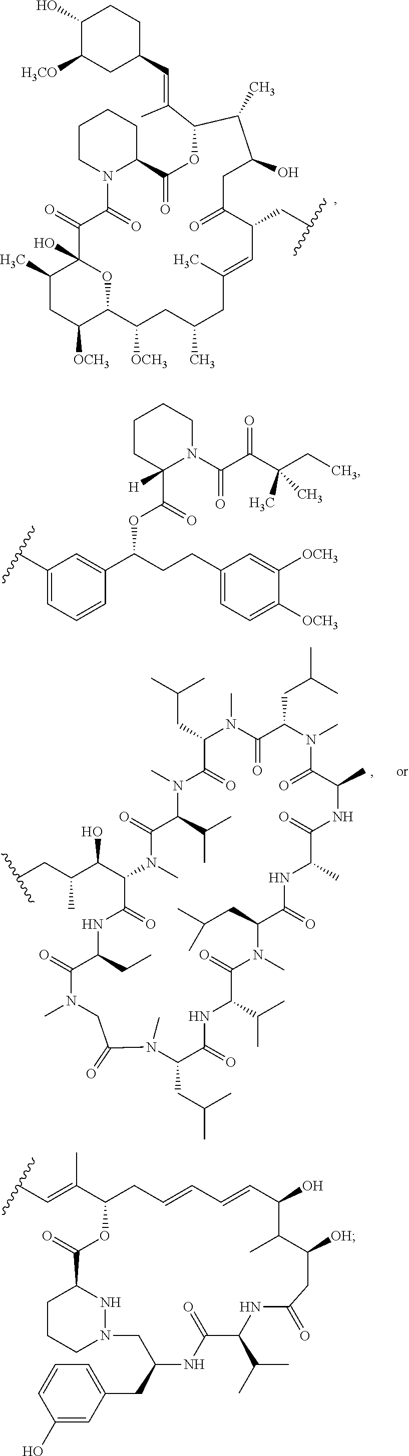

or an analog thereof.

In embodiments, the immunophilin-binding moiety is

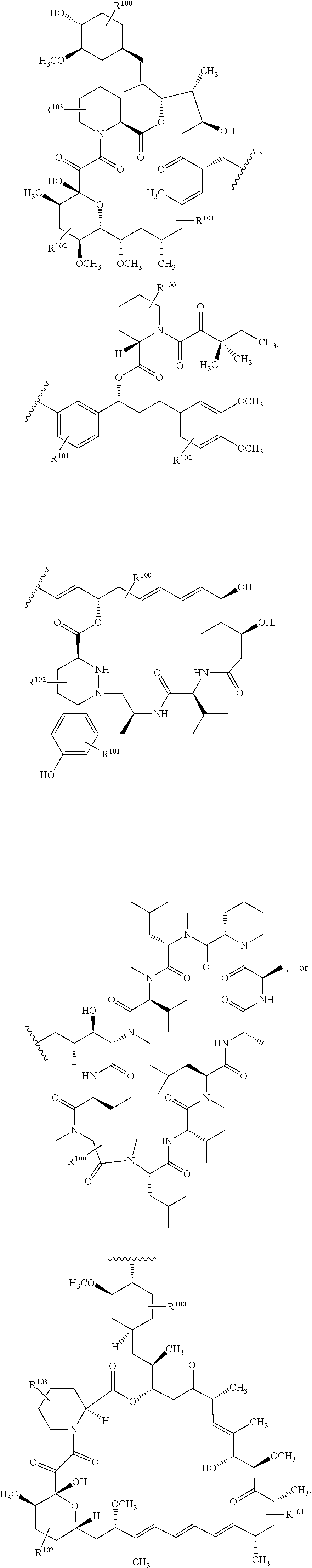

wherein R¹⁰⁰, R¹⁰¹, R¹⁰², and R¹⁰³ are as described herein and may be bonded to any atom in the ring (R¹⁰⁰, R¹⁰¹, and R¹⁰², and R¹⁰³ are floating substituents). In embodiments, the immunophilin-binding moiety is

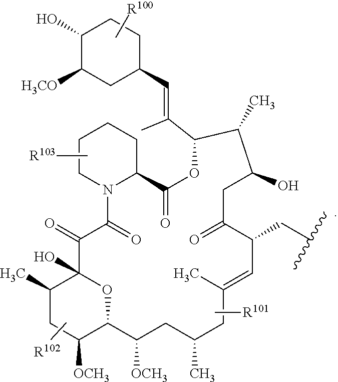

R¹⁰⁰, R¹⁰¹, R¹⁰², and R¹⁰³ are as described herein. In embodiments, the immunophilin-binding moiety is

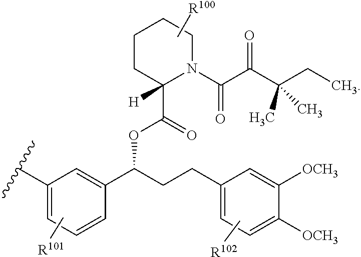

R¹⁰⁰, R¹⁰¹, and R¹⁰² are as described herein. In embodiments, the immunophilin-binding moiety is

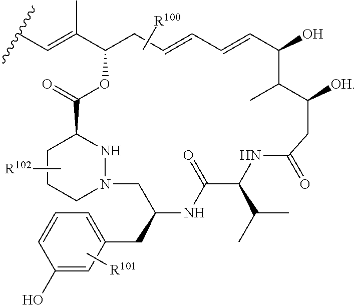

R¹⁰⁰, R¹⁰¹, and R¹⁰² are as described herein. In embodiments, the immunophilin-binding moiety is

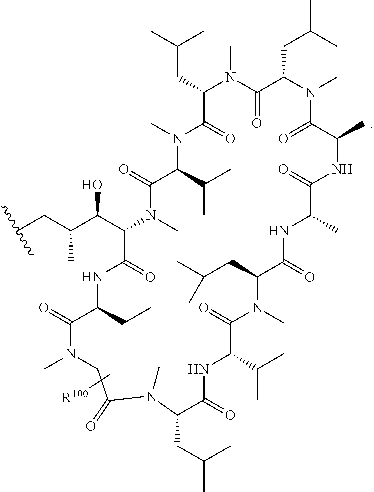

R¹⁰⁰ is as described herein. In embodiments, the immunophilin-binding moiety is

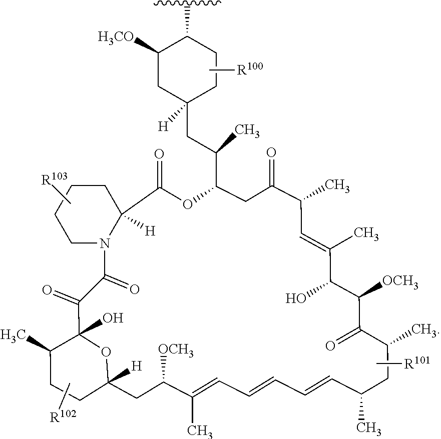

R¹⁰⁰, R¹⁰¹, R¹⁰², and R¹⁰³ are as described herein.

R¹⁰⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹⁰⁰ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁰⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰⁰ is substituted with a plurality of groups selected from substituent groups, size-limited substituent groups, and lower substituent groups; each substituent group, size-limited substituent group, and/or lower substituent group may optionally be different. In embodiments, when R¹⁰⁰ is substituted, it is substituted with at least one substituent group. In embodiments, when R¹⁰⁰ is substituted, it is substituted with at least one size-limited substituent group. In embodiments, when R¹⁰⁰ is substituted, it is substituted with at least one lower substituent group.

In embodiments, R¹⁰⁰ is independently hydrogen. In embodiments, R¹⁰⁰ is independently halogen. In embodiments, R¹⁰⁰ is independently —CCl₃. In embodiments, R¹⁰⁰ is independently —CBr₃. In embodiments, R¹⁰⁰ is independently —CF₃. In embodiments, R¹⁰⁰ is independently —CI₃. In embodiments, R¹⁰⁰ is independently —CH₂Cl. In embodiments, R¹⁰⁰ is independently —CH₂Br. In embodiments, R¹⁰⁰ is independently —CH₂F. In embodiments, R¹⁰⁰ is independently —CH₂I. In embodiments, R¹⁰⁰ is independently —CHCl₂. In embodiments, R¹⁰⁰ is independently —CHBr₂. In embodiments, R¹⁰⁰ is independently —CHF₂. In embodiments, R¹⁰⁰ is independently —CHI₂. In embodiments, R¹⁰⁰ is independently —CN. In embodiments, R¹⁰⁰ is independently —OH. In embodiments, R¹⁰⁰ is independently —NH₂. In embodiments, R¹⁰⁰ is independently —COOH. In embodiments, R¹⁰⁰ is independently —CONH₂. In embodiments, R¹⁰⁰ is independently —NO₂. In embodiments, R¹⁰⁰ is independently —SH. In embodiments, R¹⁰⁰ is independently —SO₃H. In embodiments, R¹⁰⁰ is independently —SO₄H. In embodiments, R¹⁰⁰ is independently —SO₂NH₂. In embodiments, R¹⁰⁰ is independently —NHNH₂. In embodiments, R¹⁰⁰ is independently —ONH₂. In embodiments, R¹⁰⁰ is independently —NHC(O)NHNH₂. In embodiments, R¹⁰⁰ is independently —NHC(O)NH₂. In embodiments, R¹⁰⁰ is independently —NHSO₂H. In embodiments, R¹⁰⁰ is independently —NHC(O)H. In embodiments, R¹⁰⁰ is independently —NHC(O)OH. In embodiments, R¹⁰⁰ is independently —NHC(NH)H. In embodiments, R¹⁰⁰ is independently —NHC(NH)NH₂. In embodiments, R¹⁰⁰ is independently —NHOH. In embodiments, R¹⁰⁰ is independently —OCCl₃. In embodiments, R¹⁰⁰ is independently —OCBr₃. In embodiments, R¹⁰⁰ is independently —OCF₃. In embodiments, R¹⁰⁰ is independently —OCI₃. In embodiments, R¹⁰⁰ is independently —OCH₂Cl. In embodiments, R¹⁰⁰ is independently —OCH₂Br. In embodiments, R¹⁰⁰ is independently —OCH₂F. In embodiments, R¹⁰⁰ is independently —OCH₂I. In embodiments, R¹⁰⁰ is independently —OCHCl₂. In embodiments, R¹⁰⁰ is independently —OCHBr₂. In embodiments, R¹⁰⁰ is independently —OCHF₂. In embodiments, R¹⁰⁰ is independently —OCHI₂. In embodiments, R¹⁰⁰ is independently —N₃. In embodiments, R¹⁰⁰ is independently substituted or unsubstituted alkyl (e.g., C₁-C₈, C₁-C₆, C₁-C₄, or C₁-C₂). In embodiments, R¹⁰⁰ is independently substituted alkyl (e.g., C₁-C₈, C₁-C₆, C₁-C₄, or C₁-C₂). In embodiments, R¹⁰⁰ is independently unsubstituted alkyl (e.g., C₁-C₈, C₁-C₆, C₁-C₄, or C₁-C₂). In embodiments, R¹⁰⁰ is independently substituted or unsubstituted heteroalkyl (e.g., 2 to 8 membered, 2 to 6 membered, 4 to 6 membered, 2 to 3 membered, or 4 to 5 membered). In embodiments, R¹⁰⁰ is independently substituted heteroalkyl (e.g., 2 to 8 membered, 2 to 6 membered, 4 to 6 membered, 2 to 3 membered, or 4 to 5 membered). In embodiments, R¹⁰⁰ is independently unsubstituted heteroalkyl (e.g., 2 to 8 membered, 2 to 6 membered, 4 to 6 membered, 2 to 3 membered, or 4 to 5 membered). In embodiments, R¹⁰⁰ is independently substituted or unsubstituted cycloalkyl (e.g., C₃-C₈, C₃-C₆, C₄-C₆, or C₅-C₆). In embodiments, R¹⁰⁰ is independently substituted cycloalkyl (e.g., C₃-C₈, C₃-C₆, C₄-C₆, or C₅-C₆). In embodiments, R¹⁰⁰ is independently unsubstituted cycloalkyl (e.g., C₃-C₈, C₃-C₆, C₄-C₆, or C₅-C₆). In embodiments, R¹⁰⁰ is independently substituted or unsubstituted heterocycloalkyl (e.g., 3 to 8 membered, 3 to 6 membered, 4 to 6 membered, 4 to 5 membered, or 5 to 6 membered). In embodiments, R¹⁰⁰ is independently substituted heterocycloalkyl (e.g., 3 to 8 membered, 3 to 6 membered, 4 to 6 membered, 4 to 5 membered, or 5 to 6 membered). In embodiments, R¹⁰⁰ is independently unsubstituted heterocycloalkyl (e.g., 3 to 8 membered, 3 to 6 membered, 4 to 6 membered, 4 to 5 membered, or 5 to 6 membered). In embodiments, R¹⁰⁰ is independently substituted or unsubstituted aryl (e.g., C₆-C₁₀ or phenyl). In embodiments, R¹⁰⁰ is independently substituted aryl (e.g., C₆-C₁₀ or phenyl). In embodiments, R¹⁰⁰ is independently unsubstituted aryl (e.g., C₆-C₁₀ or phenyl). In embodiments, R¹⁰⁰ is independently substituted or unsubstituted heteroaryl (e.g., 5 to 10 membered, 5 to 9 membered, or 5 to 6 membered). In embodiments, R¹⁰⁰ is independently substituted heteroaryl (e.g., 5 to 10 membered, 5 to 9 membered, or 5 to 6 membered). In embodiments, R¹⁰⁰ is independently unsubstituted heteroaryl (e.g., 5 to 10 membered, 5 to 9 membered, or 5 to 6 membered).

R¹⁰¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹⁰¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁰¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰¹ is substituted with a plurality of groups selected from substituent groups, size-limited substituent groups, and lower substituent groups; each substituent group, size-limited substituent group, and/or lower substituent group may optionally be different. In embodiments, when R¹⁰¹ is substituted, it is substituted with at least one substituent group. In embodiments, when R¹⁰¹ is substituted, it is substituted with at least one size-limited substituent group. In embodiments, when R¹⁰¹ is substituted, it is substituted with at least one lower substituent group.

In embodiments, R¹⁰¹ is independently hydrogen. In embodiments, R¹⁰¹ is independently halogen. In embodiments, R¹⁰¹ is independently —CCl₃. In embodiments, R¹⁰¹ is independently —CBr₃. In embodiments, R¹⁰¹ is independently —CF₃. In embodiments, R¹⁰¹ is independently —CI₃. In embodiments, R¹⁰¹ is independently —CH₂Cl. In embodiments, R¹⁰¹ is independently —CH₂Br. In embodiments, R¹⁰¹ is independently —CH₂F. In embodiments, R¹⁰¹ is independently —CH₂I. In embodiments, R¹⁰¹ is independently —CHCl₂. In embodiments, R¹⁰¹ is independently —CHBr₂. In embodiments, R¹⁰¹ is independently —CHF₂. In embodiments, R¹⁰¹ is independently —CHI₂. In embodiments, R¹⁰¹ is independently —CN. In embodiments, R¹⁰¹ is independently —OH. In embodiments, R¹⁰¹ is independently —NH₂. In embodiments, R¹⁰¹ is independently —COOH. In embodiments, R¹⁰¹ is independently —CONH₂. In embodiments, R¹⁰¹ is independently —NO₂. In embodiments, R¹⁰¹ is independently —SH. In embodiments, R¹⁰¹ is independently —SO₃H. In embodiments, R¹⁰¹ is independently —SO₄H. In embodiments, R¹⁰¹ is independently —SO₂NH₂. In embodiments, R¹⁰¹ is independently —NHNH₂. In embodiments, R¹⁰¹ is independently —ONH₂. In embodiments, R¹⁰¹ is independently —NHC(O)NHNH₂. In embodiments, R¹⁰¹ is independently —NHC(O)NH₂. In embodiments, R¹⁰¹ is independently —NHSO₂H. In embodiments, R¹⁰¹ is independently —NHC(O)H. In embodiments, R¹⁰¹ is independently —NHC(O)OH. In embodiments, R¹⁰¹ is independently —NHC(NH)H. In embodiments, R¹⁰¹ is independently —NHC(NH)NH₂. In embodiments, R¹⁰¹ is independently —NHOH. In embodiments, R¹⁰¹ is independently —OCCl₃. In embodiments, R¹⁰¹ is independently —OCBr₃. In embodiments, R¹⁰¹ is independently —OCF₃. In embodiments, R¹⁰¹ is independently —OCI₃. In embodiments, R¹⁰¹ is independently —OCH₂Cl. In embodiments, R¹⁰¹ is independently —OCH₂Br. In embodiments, R¹⁰¹ is independently —OCH₂F. In embodiments, R¹⁰¹ is independently —OCH₂I. In embodiments, R¹⁰¹ is independently —OCHCl₂. In embodiments, R¹⁰¹ is independently —OCHBr₂. In embodiments, R¹⁰¹ is independently —OCHF₂. In embodiments, R¹⁰¹ is independently —OCHI₂. In embodiments, R¹⁰¹ is independently —N₃. In embodiments, R¹⁰¹ is independently substituted or unsubstituted alkyl (e.g., C₁-C₈, C₁-C₆, C₁-C₄, or C₁-C₂). In embodiments, R¹⁰¹ is independently substituted alkyl (e.g., C₁-C₈, C₁-C₆, C₁-C₄, or C₁-C₂). In embodiments, R¹⁰¹ is independently unsubstituted alkyl (e.g., C₁-C₈, C₁-C₆, C₁-C₄, or C₁-C₂). In embodiments, R¹⁰¹ is independently substituted or unsubstituted heteroalkyl (e.g., 2 to 8 membered, 2 to 6 membered, 4 to 6 membered, 2 to 3 membered, or 4 to 5 membered). In embodiments, R¹⁰¹ is independently substituted heteroalkyl (e.g., 2 to 8 membered, 2 to 6 membered, 4 to 6 membered, 2 to 3 membered, or 4 to 5 membered). In embodiments, R¹⁰¹ is independently unsubstituted heteroalkyl (e.g., 2 to 8 membered, 2 to 6 membered, 4 to 6 membered, 2 to 3 membered, or 4 to 5 membered). In embodiments, R¹⁰¹ is independently substituted or unsubstituted cycloalkyl (e.g., C₃-C₈, C₃-C₆, C₄-C₆, or C₅-C₆). In embodiments, R¹⁰¹ is independently substituted cycloalkyl (e.g., C₃-C₈, C₃-C₆, C₄-C₆, or C₅-C₆). In embodiments, R¹⁰¹ is independently unsubstituted cycloalkyl (e.g., C₃-C₈, C₃-C₆, C₄-C₆, or C₅-C₆). In embodiments, R¹⁰¹ is independently substituted or unsubstituted heterocycloalkyl (e.g., 3 to 8 membered, 3 to 6 membered, 4 to 6 membered, 4 to 5 membered, or 5 to 6 membered). In embodiments, R¹⁰¹ is independently substituted heterocycloalkyl (e.g., 3 to 8 membered, 3 to 6 membered, 4 to 6 membered, 4 to 5 membered, or 5 to 6 membered). In embodiments, R¹⁰¹ is independently unsubstituted heterocycloalkyl (e.g., 3 to 8 membered, 3 to 6 membered, 4 to 6 membered, 4 to 5 membered, or 5 to 6 membered). In embodiments, R¹⁰¹ is independently substituted or unsubstituted aryl (e.g., C₆-C₁₀ or phenyl). In embodiments, R¹⁰¹ is independently substituted aryl (e.g., C₆-C₁₀ or phenyl). In embodiments, R¹⁰¹ is independently unsubstituted aryl (e.g., C₆-C₁₀ or phenyl). In embodiments, R¹⁰¹ is independently substituted or unsubstituted heteroaryl (e.g., 5 to 10 membered, 5 to 9 membered, or 5 to 6 membered). In embodiments, R¹⁰¹ is independently substituted heteroaryl (e.g., 5 to 10 membered, 5 to 9 membered, or 5 to 6 membered). In embodiments, R¹⁰¹ is independently unsubstituted heteroaryl (e.g., 5 to 10 membered, 5 to 9 membered, or 5 to 6 membered).

R¹⁰² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹⁰² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰² is substituted with a plurality of groups selected from substituent groups, size-limited substituent groups, and lower substituent groups; each substituent group, size-limited substituent group, and/or lower substituent group may optionally be different. In embodiments, when R¹⁰² is substituted, it is substituted with at least one substituent group. In embodiments, when R¹⁰² is substituted, it is substituted with at least one size-limited substituent group. In embodiments, when R¹⁰² is substituted, it is substituted with at least one lower substituent group.

In embodiments, R¹⁰² is independently hydrogen. In embodiments, R¹⁰² is independently halogen. In embodiments, R¹⁰² is independently —CCl₃. In embodiments, R¹⁰² is independently —CBr₃. In embodiments, R¹⁰² is independently —CF₃. In embodiments, R¹⁰² is independently —CI₃. In embodiments, R¹⁰² is independently —CH₂Cl. In embodiments, R¹⁰² is independently —CH₂Br. In embodiments, R¹⁰² is independently —CH₂F. In embodiments, R¹⁰² is independently —CH₂I. In embodiments, R¹⁰² is independently —CHCl₂. In embodiments, R¹⁰² is independently —CHBr₂. In embodiments, R¹⁰² is independently —CHF₂. In embodiments, R¹⁰² is independently —CHI₂. In embodiments, R¹⁰² is independently —CN. In embodiments, R¹⁰² is independently —OH. In embodiments, R¹⁰² is independently —NH₂. In embodiments, R¹⁰² is independently —COOH. In embodiments, R¹⁰² is independently —CONH₂. In embodiments, R¹⁰² is independently —NO₂. In embodiments, R¹⁰² is independently —SH. In embodiments, R¹⁰² is independently —SO₃H. In embodiments, R¹⁰² is independently —SO₄H. In embodiments, R¹⁰² is independently —SO₂NH₂. In embodiments, R¹⁰² is independently —NHNH₂. In embodiments, R¹⁰² is independently —ONH₂. In embodiments, R¹⁰² is independently —NHC(O)NHNH₂. In embodiments, R¹⁰² is independently —NHC(O)NH₂. In embodiments, R¹⁰² is independently —NHSO₂H. In embodiments, R¹⁰² is independently —NHC(O)H. In embodiments, R¹⁰² is independently —NHC(O)OH. In embodiments, R¹⁰² is independently —NHC(NH)H. In embodiments, R¹⁰² is independently —NHC(NH)NH₂. In embodiments, R¹⁰² is independently —NHOH. In embodiments, R¹⁰² is independently —OCCl₃. In embodiments, R¹⁰² is independently —OCBr₃. In embodiments, R¹⁰² is independently —OCF₃. In embodiments, R¹⁰² is independently —OCI₃. In embodiments, R¹⁰² is independently —OCH₂Cl. In embodiments, R¹⁰² is independently —OCH₂Br. In embodiments, R¹⁰² is independently —OCH₂F. In embodiments, R¹⁰² is independently —OCH₂I. In embodiments, R¹⁰² is independently —OCHCl₂. In embodiments, R¹⁰² is independently —OCHBr₂. In embodiments, R¹⁰² is independently —OCHF₂. In embodiments, R¹⁰² is independently —OCHI₂. In embodiments, R¹⁰² is independently —N₃. In embodiments, R¹⁰² is independently substituted or unsubstituted alkyl (e.g., C₁-C₈, C₁-C₆, C₁-C₄, or C₁-C₂). In embodiments, R¹⁰² is independently substituted alkyl (e.g., C₁-C₈, C₁-C₆, C₁-C₄, or C₁-C₂). In embodiments, R¹⁰² is independently unsubstituted alkyl (e.g., C₁-C₈, C₁-C₆, C₁-C₄, or C₁-C₂). In embodiments, R¹⁰² is independently substituted or unsubstituted heteroalkyl (e.g., 2 to 8 membered, 2 to 6 membered, 4 to 6 membered, 2 to 3 membered, or 4 to 5 membered). In embodiments, R¹⁰² is independently substituted heteroalkyl (e.g., 2 to 8 membered, 2 to 6 membered, 4 to 6 membered, 2 to 3 membered, or 4 to 5 membered). In embodiments, R¹⁰² is independently unsubstituted heteroalkyl (e.g., 2 to 8 membered, 2 to 6 membered, 4 to 6 membered, 2 to 3 membered, or 4 to 5 membered). In embodiments, R¹⁰² is independently substituted or unsubstituted cycloalkyl (e.g., C₃-C₈, C₃-C₆, C₄-C₆, or C₅-C₆). In embodiments, R¹⁰² is independently substituted cycloalkyl (e.g., C₃-C₈, C₃-C₆, C₄-C₆, or C₅-C₆). In embodiments, R¹⁰² is independently unsubstituted cycloalkyl (e.g., C₃-C₈, C₃-C₆, C₄-C₆, or C₅-C₆). In embodiments, R¹⁰² is independently substituted or unsubstituted heterocycloalkyl (e.g., 3 to 8 membered, 3 to 6 membered, 4 to 6 membered, 4 to 5 membered, or 5 to 6 membered). In embodiments, R¹⁰² is independently substituted heterocycloalkyl (e.g., 3 to 8 membered, 3 to 6 membered, 4 to 6 membered, 4 to 5 membered, or 5 to 6 membered). In embodiments, R¹⁰² is independently unsubstituted heterocycloalkyl (e.g., 3 to 8 membered, 3 to 6 membered, 4 to 6 membered, 4 to 5 membered, or 5 to 6 membered). In embodiments, R¹⁰² is independently substituted or unsubstituted aryl (e.g., C₆-C₁₀ or phenyl). In embodiments, R¹⁰² is independently substituted aryl (e.g., C₆-C₁₀ or phenyl). In embodiments, R¹⁰² is independently unsubstituted aryl (e.g., C₆-C₁₀ or phenyl). In embodiments, R¹⁰² is independently substituted or unsubstituted heteroaryl (e.g., 5 to 10 membered, 5 to 9 membered, or 5 to 6 membered). In embodiments, R¹⁰² is independently substituted heteroaryl (e.g., 5 to 10 membered, 5 to 9 membered, or 5 to 6 membered). In embodiments, R¹⁰² is independently unsubstituted heteroaryl (e.g., 5 to 10 membered, 5 to 9 membered, or 5 to 6 membered).

R¹⁰³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹⁰³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHC(NH)H, —NHC(NH)NH₂, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R¹⁰³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰³ is substituted with a plurality of groups selected from substituent groups, size-limited substituent groups, and lower substituent groups; each substituent group, size-limited substituent group, and/or lower substituent group may optionally be different. In embodiments, when R¹⁰³ is substituted, it is substituted with at least one substituent group. In embodiments, when R¹⁰³ is substituted, it is substituted with at least one size-limited substituent group. In embodiments, when R¹⁰³ is substituted, it is substituted with at least one lower substituent group.

In embodiments, R¹⁰³ is independently hydrogen. In embodiments, R¹⁰³ is independently halogen. In embodiments, R¹⁰³ is independently —CCl₃. In embodiments, R¹⁰³ is independently —CBr₃. In embodiments, R¹⁰³ is independently —CF₃. In embodiments, R¹⁰³ is independently —CI₃. In embodiments, R¹⁰³ is independently —CH₂Cl. In embodiments, R¹⁰³ is independently —CH₂Br. In embodiments, R¹⁰³ is independently —CH₂F. In embodiments, R¹⁰³ is independently —CH₂I. In embodiments, R¹⁰³ is independently —CHCl₂. In embodiments, R¹⁰³ is independently —CHBr₂. In embodiments, R¹⁰³ is independently —CHF₂. In embodiments, R¹⁰³ is independently —CHI₂. In embodiments, R¹⁰³ is independently —CN. In embodiments, R¹⁰³ is independently —OH. In embodiments, R¹⁰³ is independently —NH₂. In embodiments, R¹⁰³ is independently —COOH. In embodiments, R¹⁰³ is independently —CONH₂. In embodiments, R¹⁰³ is independently —NO₂. In embodiments, R¹⁰³ is independently —SH. In embodiments, R¹⁰³ is independently —SO₃H. In embodiments, R¹⁰³ is independently —SO₄H. In embodiments, R¹⁰³ is independently —SO₂NH₂. In embodiments, R¹⁰3 is independently —NHNH₂. In embodiments, R¹⁰³ is independently —ONH₂. In embodiments, R¹⁰³ is independently —NHC(O)NHNH₂. In embodiments, R¹⁰³ is independently —NHC(O)NH₂. In embodiments, R¹⁰³ is independently —NHSO₂H. In embodiments, R¹⁰³ is independently —NHC(O)H. In embodiments, R¹⁰³ is independently —NHC(O)OH. In embodiments, R¹⁰³ is independently —NHC(NH)H. In embodiments, R¹⁰³ is independently —NHC(NH)NH₂. In embodiments, R¹⁰³ is independently —NHOH. In embodiments, R¹⁰³ is independently —OCCl₃. In embodiments, R¹⁰³ is independently —OCBr₃. In embodiments, R¹⁰³ is independently —OCF₃. In embodiments, R¹⁰³ is independently —OCI₃. In embodiments, R¹⁰³ is independently —OCH₂Cl. In embodiments, R¹⁰³ is independently —OCH₂Br. In embodiments, R¹⁰³ is independently —OCH₂F. In embodiments, R¹⁰³ is independently —OCH₂I. In embodiments, R¹⁰³ is independently —OCHCl₂. In embodiments, R¹⁰³ is independently —OCHBr₂. In embodiments, R¹⁰³ is independently —OCHF₂. In embodiments, R¹⁰³ is independently —OCHI₂. In embodiments, R¹⁰³ is independently —N₃. In embodiments, R¹⁰³ is independently substituted or unsubstituted alkyl (e.g., C₁-C₈, C₁-C₆, C₁-C₄, or C₁-C₂). In embodiments, R¹⁰³ is independently substituted alkyl (e.g., C₁-C₈, C₁-C₆, C₁-C₄, or C₁-C₂). In embodiments, R¹⁰³ is independently unsubstituted alkyl (e.g., C₁-C₈, C₁-C₆, C₁-C₄, or C₁-C₂). In embodiments, R¹⁰³ is independently substituted or unsubstituted heteroalkyl (e.g., 2 to 8 membered, 2 to 6 membered, 4 to 6 membered, 2 to 3 membered, or 4 to 5 membered). In embodiments, R¹⁰³ is independently substituted heteroalkyl (e.g., 2 to 8 membered, 2 to 6 membered, 4 to 6 membered, 2 to 3 membered, or 4 to 5 membered). In embodiments, R¹⁰³ is independently unsubstituted heteroalkyl (e.g., 2 to 8 membered, 2 to 6 membered, 4 to 6 membered, 2 to 3 membered, or 4 to 5 membered). In embodiments, R¹⁰³ is independently substituted or unsubstituted cycloalkyl (e.g., C₃-C₈, C₃-C₆, C₄-C₆, or C₅-C₆). In embodiments, R¹⁰³ is independently substituted cycloalkyl (e.g., C₃-C₈, C₃-C₆, C₄-C₆, or C₅-C₆). In embodiments, R¹⁰³ is independently unsubstituted cycloalkyl (e.g., C₃-C₈, C₃-C₆, C₄-C₆, or C₅-C₆). In embodiments, R¹⁰³ is independently substituted or unsubstituted heterocycloalkyl (e.g., 3 to 8 membered, 3 to 6 membered, 4 to 6 membered, 4 to 5 membered, or 5 to 6 membered). In embodiments, R¹⁰³ is independently substituted heterocycloalkyl (e.g., 3 to 8 membered, 3 to 6 membered, 4 to 6 membered, 4 to 5 membered, or 5 to 6 membered). In embodiments, R¹⁰³ is independently unsubstituted heterocycloalkyl (e.g., 3 to 8 membered, 3 to 6 membered, 4 to 6 membered, 4 to 5 membered, or 5 to 6 membered). In embodiments, R¹⁰³ is independently substituted or unsubstituted aryl (e.g., C₆-C₁₀ or phenyl). In embodiments, R¹⁰³ is independently substituted aryl (e.g., C₆-C₁₀ or phenyl). In embodiments, R¹⁰³ is independently unsubstituted aryl (e.g., C₆-C₁₀ or phenyl). In embodiments, R¹⁰³ is independently substituted or unsubstituted heteroaryl (e.g., 5 to 10 membered, 5 to 9 membered, or 5 to 6 membered). In embodiments, R¹⁰³ is independently substituted heteroaryl (e.g., 5 to 10 membered, 5 to 9 membered, or 5 to 6 membered). In embodiments, R¹⁰³ is independently unsubstituted heteroaryl (e.g., 5 to 10 membered, 5 to 9 membered, or 5 to 6 membered).

In embodiments, L¹ is L²-L³-L⁴-L⁵-L⁶.

L² is connected directly to the moiety of an immunophilin-binding compound.

L² is a bond, —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

L³ is a bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

L⁴ is a bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

L⁵ is a

bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

L⁶ is a

bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

R², R³, R⁴, R⁵, and R⁶ are independently hydrogen, halogen, —CCl₃, —CBr₃, —CF₃, —CI₃,

—CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, L²

is —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

In embodiments, L²

is —S(O)₂—, —N(R²⁶)—, —O—, —S—, —C(O)—, —C(O)N(R²⁶)—, —N(R²⁶)C(O)—, —N(R²⁶)C(O)NH—, —NHC(O)N(R²⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

In embodiments, L³ is a

bond, —S(O)₂—, —N(R²⁹)—, —O—, —S—, —C(O)—, —C(O)N(R²⁹)—, —N(R²⁹)C(O)—, —N(R²⁹)C(O)NH—, —NHC(O)N(R²⁹)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

In embodiments, L⁴ is a

bond, —S(O)₂—, —N(R³²)—, —O—, —S—, —C(O)—, —C(O)N(R³²)—, —N(R³²)C(O)—, —N(R³²)C(O)NH—, —NHC(O)N(R³²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

In embodiments, L⁵ is a

bond, —S(O)₂—, —N(R³⁵)—, —O—, —S—, —C(O)—, —C(O)N(R³⁵)—, —N(R³⁵)C(O)—, —N(R³⁵)C(O)NH—, —N HC(O)N(R³⁵)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

In embodiments, L⁶ is a

bond, —S(O)₂—, —N(R³⁸)—, —O—, —S—, —C(O)—, —C(O)N(R³⁸)—, —N(R³⁸)C(O)—, —N(R³⁸)C(O)NH—, —N HC(O)N(R³⁸)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or bioconjugate linker.

In embodiments, L²

is —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L² is —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀, C₁₀, or phenylene), or substituted or unsubstituted heteroarylene (e.g., 5 to 10 membered, 5 to 9 membered, or 5 to 6 membered).

In embodiments, L² is —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted (e.g., substituted with at least one substituent group, size-limited substituent group, or lower substituent group) or unsubstituted alkylene (e.g., C₁-C₈, C₁-C₆, or C₁-C₄), substituted (e.g., substituted with at least one substituent group, size-limited substituent group, or lower substituent group) or unsubstituted heteroalkylene (e.g., 2 to 8 membered, 2 to 6 membered, or 2 to 4 membered), substituted (e.g., substituted with at least one substituent group, size-limited substituent group, or lower substituent group) or unsubstituted cycloalkylene (e.g., C₃-C₈, C₃-C₆, or C₅-C₆), substituted (e.g., substituted with at least one substituent group, size-limited substituent group, or lower substituent group) or unsubstituted heterocycloalkylene (e.g., 3 to 8 membered, 3 to 6 membered, or 5 to 6 membered), substituted (e.g., substituted with at least one substituent group, size-limited substituent group, or lower substituent group) or unsubstituted arylene (e.g., C₆-C₁₀, C₁₀, or phenylene), or substituted (e.g., substituted with at least one substituent group, size-limited substituent group, or lower substituent group) or unsubstituted heteroarylene (e.g., 5 to 10 membered, 5 to 9 membered, or 5 to 6 membered).

In embodiments, a substituted L² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted or unsubstituted C₁-C₆ alkylene, or substituted or unsubstituted 2 to 6 membered heteroalkylene.

In embodiments, R² is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, L³ is a

bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L³ is a bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, —OC(O)—, substituted (e.g., substituted with at least one substituent group, size-limited substituent group, or lower substituent group) or unsubstituted alkylene (e.g., C₁-C₈, C₁-C₆, or C₁-C₄), substituted (e.g., substituted with at least one substituent group, size-limited substituent group, or lower substituent group) or unsubstituted heteroalkylene (e.g., 2 to 8 membered, 2 to 6 membered, or 2 to 4 membered), substituted (e.g., substituted with at least one substituent group, size-limited substituent group, or lower substituent group) or unsubstituted cycloalkylene (e.g., C₃-C₈, C₃-C₆, or C₅-C₆), substituted (e.g., substituted with at least one substituent group, size-limited substituent group, or lower substituent group) or unsubstituted heterocycloalkylene (e.g., 3 to 8 membered, 3 to 6 membered, or 5 to 6 membered), substituted (e.g., substituted with at least one substituent group, size-limited substituent group, or lower substituent group) or unsubstituted arylene (e.g., C₆-C₁₀, C₁₀, or phenylene), or substituted (e.g., substituted with at least one substituent group, size-limited substituent group, or lower substituent group) or unsubstituted heteroarylene (e.g., 5 to 10 membered, 5 to 9 membered, or 5 to 6 membered).

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, —OC(O)—, substituted or unsubstituted C₁-C₆ alkylene, or substituted or unsubstituted 2 to 6 membered heteroalkylene.

In embodiments, R³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, L⁴ is a

bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L⁴ is a bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, substituted (e.g., substituted with at least one substituent group, size-limited substituent group, or lower substituent group) or unsubstituted alkylene (e.g., C₁-C₈, C₁-C₆, or C₁-C₄), substituted (e.g., substituted with at least one substituent group, size-limited substituent group, or lower substituent group) or unsubstituted heteroalkylene (e.g., 2 to 8 membered, 2 to 6 membered, or 2 to 4 membered), substituted (e.g., substituted with at least one substituent group, size-limited substituent group, or lower substituent group) or unsubstituted cycloalkylene (e.g., C₃-C₈, C₃-C₆, or C₅-C₆), substituted (e.g., substituted with at least one substituent group, size-limited substituent group, or lower substituent group) or unsubstituted heterocycloalkylene (e.g., 3 to 8 membered, 3 to 6 membered, or 5 to 6 membered), substituted (e.g., substituted with at least one substituent group, size-limited substituent group, or lower substituent group) or unsubstituted arylene (e.g., C₆-C₁₀, C₁₀, or phenylene), or substituted (e.g., substituted with at least one substituent group, size-limited substituent group, or lower substituent group) or unsubstituted heteroarylene (e.g., 5 to 10 membered, 5 to 9 membered, or 5 to 6 membered).

In embodiments, a substituted L⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁴ is substituted with a plurality of groups selected from substituent groups, size-limited substituent groups, and lower substituent groups; each substituent group, size-limited substituent group, and/or lower substituent group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, L⁴ is a bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, substituted or unsubstituted C₁-C₆ alkylene, or substituted or unsubstituted 2 to 6 membered heteroalkylene.

In embodiments, R⁴ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, L⁵ is a

bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L⁵ is a bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, substituted (e.g., substituted with at least one substituent group, size-limited substituent group, or lower substituent group) or unsubstituted alkylene (e.g., C₁-C₈, C₁-C₆, or C₁-C₄), substituted (e.g., substituted with at least one substituent group, size-limited substituent group, or lower substituent group) or unsubstituted heteroalkylene (e.g., 2 to 8 membered, 2 to 6 membered, or 2 to 4 membered), substituted (e.g., substituted with at least one substituent group, size-limited substituent group, or lower substituent group) or unsubstituted cycloalkylene (e.g., C₃-C₈, C₃-C₆, or C₅-C₆), substituted (e.g., substituted with at least one substituent group, size-limited substituent group, or lower substituent group) or unsubstituted heterocycloalkylene (e.g., 3 to 8 membered, 3 to 6 membered, or 5 to 6 membered), substituted (e.g., substituted with at least one substituent group, size-limited substituent group, or lower substituent group) or unsubstituted arylene (e.g., C₆-C₁₀, C₁₀, or phenylene), or substituted (e.g., substituted with at least one substituent group, size-limited substituent group, or lower substituent group) or unsubstituted heteroarylene (e.g., 5 to 10 membered, 5 to 9 membered, or 5 to 6 membered).

In embodiments, a substituted L⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁵ is substituted with a plurality of groups selected from substituent groups, size-limited substituent groups, and lower substituent groups; each substituent group, size-limited substituent group, and/or lower substituent group may optionally be different. In embodiments, when L⁵ is substituted, it is substituted with at least one substituent group. In embodiments, when L⁵ is substituted, it is substituted with at least one size-limited substituent group. In embodiments, when L⁵ is substituted, it is substituted with at least one lower substituent group.

In embodiments, L⁵ is a bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, substituted or unsubstituted C₁-C₆ alkylene, or substituted or unsubstituted 2 to 6 membered heteroalkylene.

In embodiments, R⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, L⁶ is a

bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L⁶ is a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted (e.g., substituted with at least one substituent group, size-limited substituent group, or lower substituent group) or unsubstituted alkylene (e.g., C₁-C₈, C₁-C₆, or C₁-C₄), substituted (e.g., substituted with at least one substituent group, size-limited substituent group, or lower substituent group) or unsubstituted heteroalkylene (e.g., 2 to 8 membered, 2 to 6 membered, or 2 to 4 membered), substituted (e.g., substituted with at least one substituent group, size-limited substituent group, or lower substituent group) or unsubstituted cycloalkylene (e.g., C₃-C₈, C₃-C₆, or C₅-C₆), substituted (e.g., substituted with at least one substituent group, size-limited substituent group, or lower substituent group) or unsubstituted heterocycloalkylene (e.g., 3 to 8 membered, 3 to 6 membered, or 5 to 6 membered), substituted (e.g., substituted with at least one substituent group, size-limited substituent group, or lower substituent group) or unsubstituted arylene (e.g., C₆-C₁₀, C₁₀, or phenylene), or substituted (e.g., substituted with at least one substituent group, size-limited substituent group, or lower substituent group) or unsubstituted heteroarylene (e.g., 5 to 10 membered, 5 to 9 membered, or 5 to 6 membered).

In embodiments, a substituted L⁶ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁶ is substituted with a plurality of groups selected from substituent groups, size-limited substituent groups, and lower substituent groups; each substituent group, size-limited substituent group, and/or lower substituent group may optionally be different. In embodiments, when L⁶ is substituted, it is substituted with at least one substituent group. In embodiments, when L⁶ is substituted, it is substituted with at least one size-limited substituent group. In embodiments, when L⁶ is substituted, it is substituted with at least one lower substituent group.

In embodiments, L⁶ is a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted C₁-C₆ alkylene, or substituted or unsubstituted 2 to 6 membered heteroalkylene.

In embodiments, R⁶ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, L³, L⁴, L⁵, and L⁶ are a bond.

In embodiments, L² is a substituted or unsubstituted alkylene, substituted or unsubstituted heteroalkylene, substituted or unsubstituted cycloalkylene, or substituted or unsubstituted heterocycloalkylene; L³ is a bond, substituted or unsubstituted alkylene, substituted or unsubstituted heteroalkylene, substituted or unsubstituted cycloalkylene, or substituted or unsubstituted heterocycloalkylene; L⁴ is a bond, substituted or unsubstituted alkylene, or substituted or unsubstituted heteroalkylene; L⁵ is a bond; and L⁶ is a bond.

In embodiments, L² is a substituted or unsubstituted alkylene, substituted or unsubstituted heteroalkylene, substituted or unsubstituted cycloalkylene, substituted or unsubstituted heterocycloalkylene, or bioconjugate linker.

In embodiments, L³ is a bond, substituted or unsubstituted alkylene, substituted or unsubstituted heteroalkylene, substituted or unsubstituted cycloalkylene, substituted or unsubstituted heterocycloalkylene, or bioconjugate linker.

In embodiments, L⁴ is a bond, substituted or unsubstituted alkylene, substituted or unsubstituted heteroalkylene, or bioconjugate linker.

In embodiments, L⁵ is a bond.

In embodiments, L⁶ is a bond.

In embodiments, L² is a substituted or unsubstituted alkylene, substituted or unsubstituted heteroalkylene, substituted or unsubstituted cycloalkylene, or substituted or unsubstituted heterocycloalkylene.

In embodiments, L³ is a bond, substituted or unsubstituted alkylene, substituted or unsubstituted heteroalkylene, substituted or unsubstituted cycloalkylene, or substituted or unsubstituted heterocycloalkylene.

In embodiments, L⁴ is a bond, substituted or unsubstituted alkylene, or substituted or unsubstituted heteroalkylene.

In embodiments, L² is an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene, or a bioconjugate linker; L³ is a bond, an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene, an unsubstituted 5 to 6 membered heterocycloalkylene, or a bioconjugate linker; L⁴ is a bond, an unsubstituted C₃-C₇ alkylene, an oxo-substituted C₃-C₇ alkylene, an unsubstituted 3 to 17 membered heteroalkylene, an oxo-substituted 3 to 17 membered heteroalkylene, or a bioconjugate linker; L⁵ is a bond; and L⁶ is a bond.

In embodiments, L² is an unsubstituted C₃-C₇ alkylene, an oxo-substituted C₃-C₁₀ alkylene, an unsubstituted 3 to 17 membered heteroalkylene, or an oxo-substituted 3 to 17 membered heteroalkylene; L³ is a bond, an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene, or an unsubstituted 5 to 6 membered heterocycloalkylene; L⁴ is a bond, an unsubstituted C₃-C₇ alkylene, an oxo-substituted C₃-C₁₀ alkylene, an unsubstituted 3 to 17 membered heteroalkylene, or an oxo-substituted 3 to 17 membered heteroalkylene; L⁵ is a bond; and L⁶ is a bond.

In embodiments, L² is an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene, or a bioconjugate linker.

In embodiments, L³ is a bond, an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene, an unsubstituted 5 to 6 membered heterocycloalkylene, or a bioconjugate linker.

In embodiments, L⁴ is a bond, an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene, or a bioconjugate linker.

In embodiments, L² is an unsubstituted C₃-C₇ alkylene, an oxo-substituted C₃-C₁₀ alkylene, an unsubstituted 3 to 17 membered heteroalkylene, or an oxo-substituted 3 to 17 membered heteroalkylene.

In embodiments, L³ is a bond, an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene, or an unsubstituted 5 to 6 membered heterocycloalkylene.

In embodiments, L⁴ is a bond, an unsubstituted C₃-C₇ alkylene, an oxo-substituted C₃-C₁₀ alkylene, an unsubstituted 3 to 17 membered heteroalkylene, or an oxo-substituted 3 to 17 membered heteroalkylene.

In embodiments, L¹ is a bond, an unsubstituted C₃-C₇ alkylene, an oxo-substituted C₃-C₁₀ alkylene, an unsubstituted 3 to 17 membered heteroalkylene, an oxo-substituted 3 to 17 membered heteroalkylene; or a bioconjugate linker.

In embodiments, L¹ is a bond, an unsubstituted C₃-C₇ alkylene, an oxo-substituted C₃-C₁₀ alkylene, an unsubstituted 3 to 17 membered heteroalkylene, or an oxo-substituted 3 to 17 membered heteroalkylene.

In embodiments, L¹ is

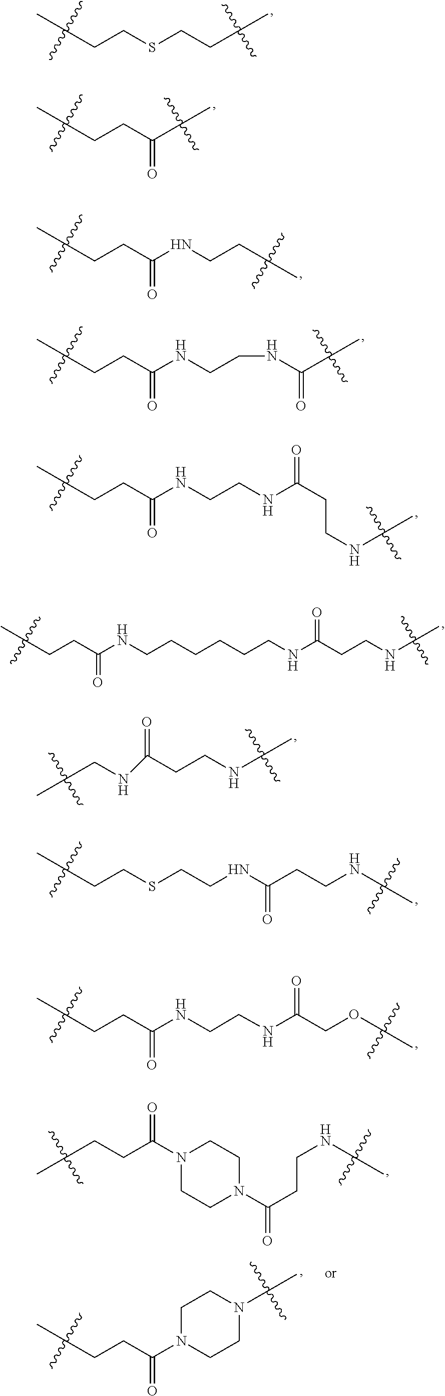

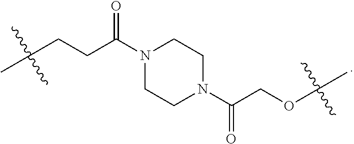 [SMILES: *CCC(=O)N1CCN(C(=O)CO*)CC1]

In embodiments, L¹ is

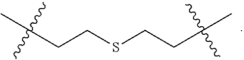 [SMILES: *CCSCC*]

In embodiments, L¹ is

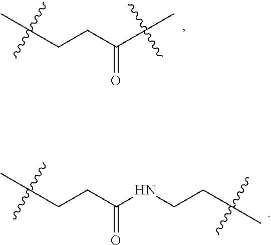 [SMILES: *CCC(*)=O ; *CCNC(=O)CC*]

In embodiments, L¹ is

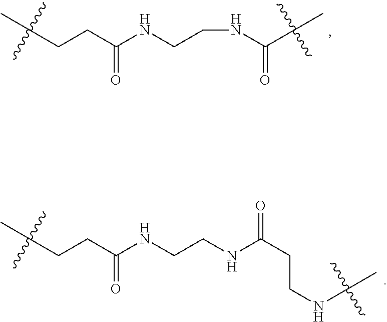 [SMILES: *CCC(=O)NCCNC(*)=O ; *CCC(=O)NCCNC(=O)CCN*]

In embodiments, L¹ is

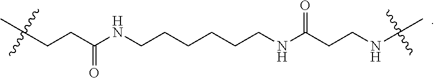 [SMILES: *CCC(=O)NCCCCCCNC(=O)CCN*]

In embodiments, L¹ is

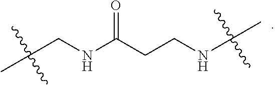 [SMILES: *CNC(=O)CCN*]

In embodiments, L¹ is

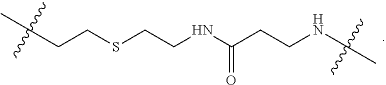 [SMILES: *CCSCCNC(=O)CCN*]

In embodiments, L¹ is

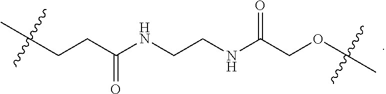 [SMILES: *CCC(=O)NCCNC(=O)CO*]

In embodiments, L¹ is

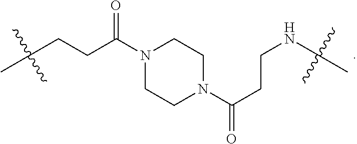 [SMILES: *CCC(=O)N1CCN(C(=O)CCN*)CC1]

In embodiments L¹ is

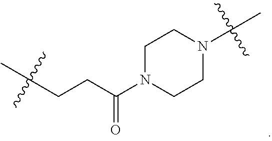 [SMILES: *CCC(=O)N1CCN(*)CC1]

In embodiments, L¹ is

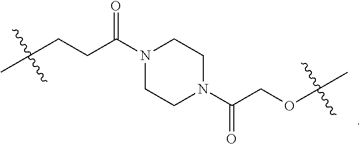 [SMILES: *CCC(=O)N1CCN(C(=O)CO*)CC1]

In embodiments, L¹ is a bond. In embodiments, L¹ is a substituted or unsubstituted alkylene or substituted or unsubstituted heteroalkylene. In embodiments, L¹ is a substituted alkylene. In embodiments, L¹ is an unsubstituted alkylene. In embodiments, L¹ is a substituted heteroalkylene. In embodiments, L¹ is an unsubstituted heteroalkylene.

In embodiments, R¹ is a monovalent kinase inhibitor, a monovalent pseudokinase inhibitor, a monovalent GTPase inhibitor, or a monovalent histone-modifying enzyme inhibitor.

In embodiments, R¹ is a monovalent kinase inhibitor.

In embodiments, the kinase is not mTOR.

In embodiments, the monovalent kinase inhibitor is a monovalent Src kinase inhibitor.

In embodiments, the monovalent Src kinase inhibitor is a monovalent dasatinib or monovalent dasatinib derivative.

In embodiments, the monovalent dasatinib derivative has the formula:

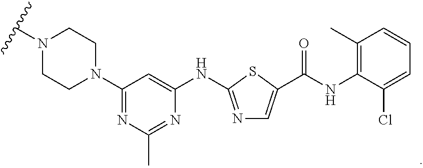 [SMILES: *N1CCN(c2cc(Nc3ncc(C(=O)Nc4c(C)cccc4Cl)s3)nc(C)n2)CC1]

In embodiments, the monovalent kinase inhibitor is a monovalent Raf inhibitor, VEGFR inhibitor, PDGFR inhibitor, or c-Kit inhibitor.

In embodiments, the monovalent Raf inhibitor, VEGFR inhibitor, PDGFR inhibitor, or c-Kit inhibitor is a monovalent sorafenib or monovalent sorafenib derivative.

In embodiments, the monovalent sorafenib derivative has the formula:

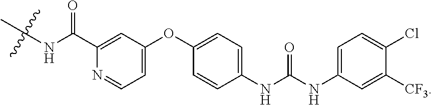 [SMILES: *NC(=O)c1cc(Oc2ccc(NC(=O)Nc3ccc(Cl)c(C(F)(F)F)c3)cc2)ccn1]

In embodiments, the monovalent kinase inhibitor is a monovalent EGFR inhibitor.

In embodiments, the monovalent EGFR inhibitor is a monovalent lapatinib, monovalent lapatinib derivative, monovalent erlotinib, monovalent erlotinib derivative, monovalent gefitinib, or monovalent gefitinib derivative.

In embodiments, the monovalent EGFR inhibitor has the formula:

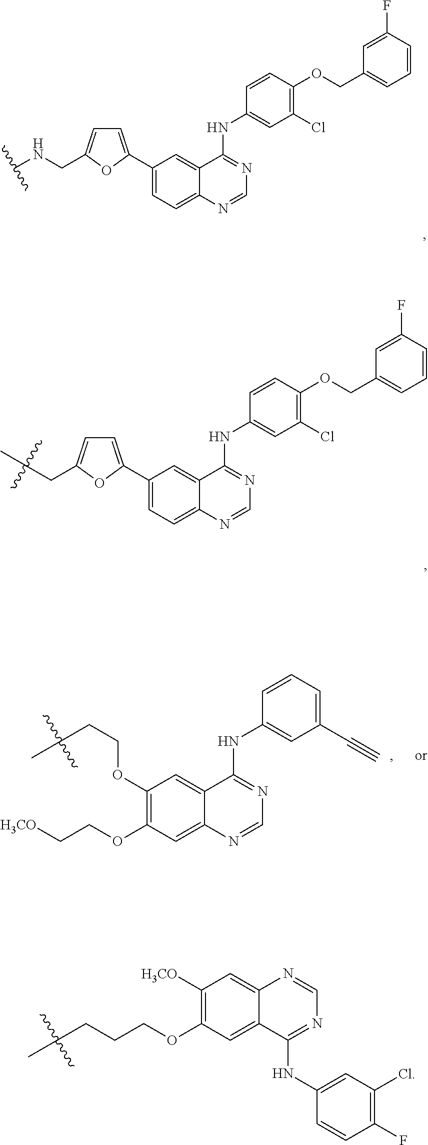 [SMILES: *NCc1ccc(-c2ccc3ncnc(Nc4ccc(OCc5cccc(F)c5)c(Cl)c4)c3c2)o1 ; *Cc1ccc(-c2ccc3ncnc(Nc4ccc(OCc5cccc(F)c5)c(Cl)c4)c3c2)o1 ; *CCOc1cc2c(Nc3cccc(C#C)c3)ncnc2cc1OCCOC ; *CCCOc1cc2c(Nc3ccc(F)c(Cl)c3)ncnc2cc1OC]

In embodiments, the monovalent kinase inhibitor is a monovalent LRRK2 inhibitor.

In embodiments, the monovalent LRRK2 inhibitor is a monovalent GNE-7915 or monovalent GNE-7915 derivative.

In embodiments, the monovalent GNE-7915 derivative has the formula:

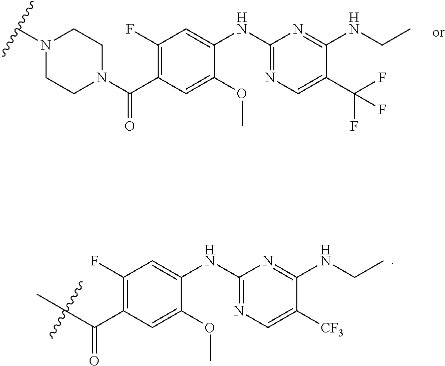 [SMILES: *N1CCN(C(=O)c2cc(OC)c(Nc3ncc(C(F)(F)F)c(NCC)n3)cc2F)CC1 ; *C(=O)c1cc(OC)c(Nc2ncc(C(F)(F)F)c(NCC)n2)cc1F]

In embodiments, the monovalent kinase inhibitor is a monovalent MAP4K inhibitor.

In embodiments, the monovalent MAP4K inhibitor is a monovalent HGK inhibitor.

In embodiments, the monovalent HGK inhibitor has the formula:

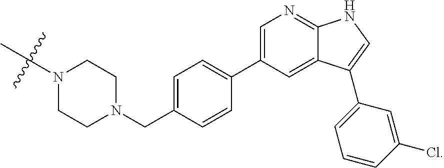 [SMILES: *N1CCN(Cc2ccc(-c3cnc4[nH]cc(-c5cccc(Cl)c5)c4c3)cc2)CC1]

In embodiments, the monovalent kinase inhibitor is a monovalent MAP3K inhibitor.

In embodiments, the monovalent MAP3K inhibitor is a monovalent DLK inhibitor.

In embodiments, the monovalent DLK inhibitor has the formula:

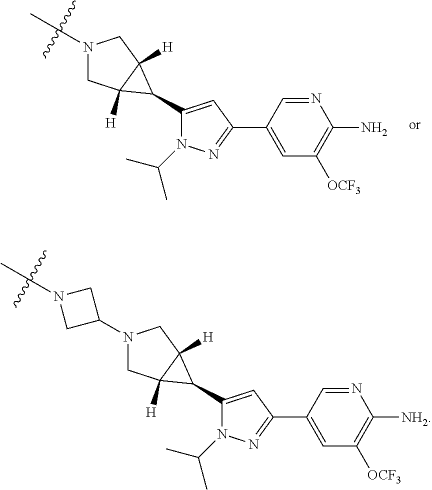 [SMILES: *N1C[C@@H]2[C@H](C1)[C@H]2c1cc(-c2cnc(N)c(OC(F)(F)F)c2)nn1C(C)C ; *N1CC(N2C[C@@H]3[C@H](C2)[C@H]3c2cc(-c3cnc(N)c(OC(F)(F)F)c3)nn2C(C)C)C1]

In embodiments, the monovalent DLK inhibitor has the formula:

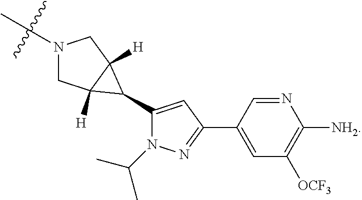 [SMILES: *N1C[C@@H]2[C@H](C1)[C@H]2c1cc(-c2cnc(N)c(OC(F)(F)F)c2)nn1C(C)C]

In embodiments, the monovalent DLK inhibitor has the formula:

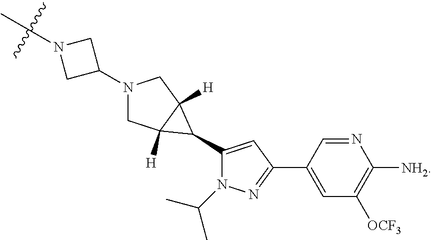 [SMILES: *N1CC(N2C[C@@H]3[C@H](C2)[C@H]3c2cc(-c3cnc(N)c(OC(F)(F)F)c3)nn2C(C)C)C1]

In embodiments, R¹ is a monovalent KRAS inhibitor.

In embodiments, the monovalent KRAS inhibitor is a monovalent KRAS G12C inhibitor or a monovalent KRAS M72C inhibitor. In embodiments, the monovalent KRAS inhibitor is a monovalent KRAS G12C inhibitor. In embodiments, the monovalent KRAS inhibitor has the formula:

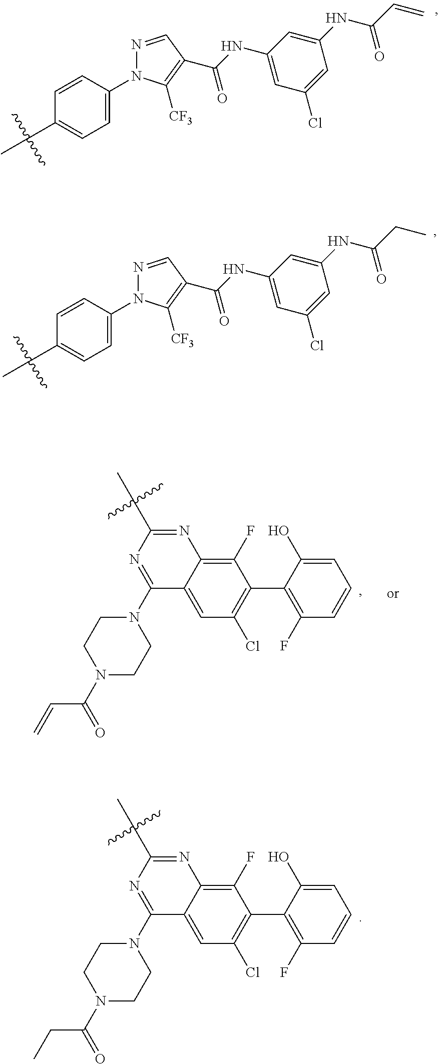

In embodiments, R¹ is a monovalent PI4K inhibitor.

In embodiments, the monovalent PI4K inhibitor has the formula:

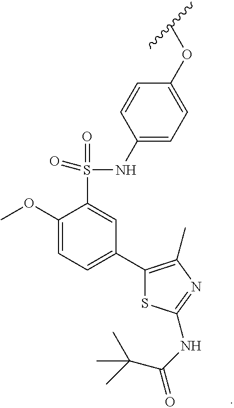 [SMILES: *Oc1ccc(NS(=O)(=O)c2cc(-c3sc(NC(=O)C(C)(C)C)nc3C)ccc2OC)cc1]

In embodiments, the monovalent PI4K inhibitor has the formula:

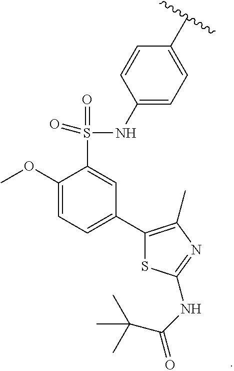 [SMILES: *c1ccc(NS(=O)(=O)c2cc(-c3sc(NC(=O)C(C)(C)C)nc3C)ccc2OC)cc1]

In embodiments, the monovalent PI4K inhibitor has the formula:

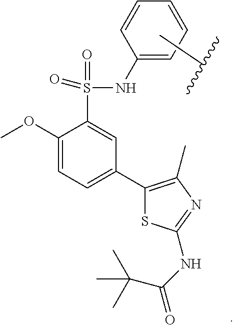

In embodiments, the monovalent PI4K inhibitor has the formula:

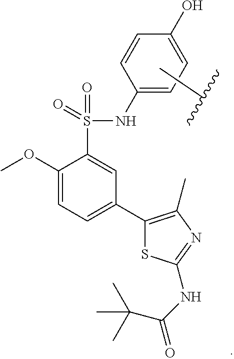

In embodiments, the monovalent PI4K inhibitor has the formula:

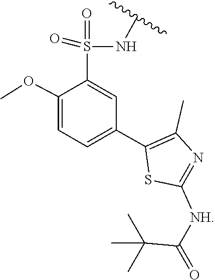 [SMILES: *NS(=O)(=O)c1cc(-c2sc(NC(=O)C(C)(C)C)nc2C)ccc1OC]

In embodiments, the monovalent PI4K inhibitor has the formula:

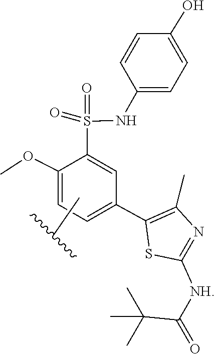

In embodiments, the compound is not a calcineurin inhibitor.

In embodiments, the covalent linker is at least or about 1.5 Å in length (e.g., at least or about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Å in length). In embodiments, the covalent linker is at least or about the length of 1 methylene groups (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 methylene groups). In embodiments, the covalent linker is at least or about the length of 5 methylene groups (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 methylene groups). In embodiments, the covalent linker is at least or about the length of 11 methylene groups (e.g., at least about or about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 methylene groups). In embodiments, the covalent linker is at least or about the length of 27 methylene groups (e.g., 27, 28, 29, 30, 31, 32, 33, 34, 35, 36, 37, 38, 39, 40, 41, 42, 43, 44, 45, 46, 47, 48, 49, or 50 methylene groups). In embodiments, the covalent linker is from about 5 to 54 Å in length. In embodiments, the covalent linker is from about 6 to 54 Å in length. In embodiments, the covalent linker is from about 7 to 54 Å in length. In embodiments, the covalent linker is from about 9 to 54 Ain length. In embodiments, the covalent linker is from about 11 to 54 Å in length. In embodiments, the covalent linker is from about 13 to 54 Å in length. In embodiments, the covalent linker is from about 15 to 54 Å in length. In embodiments, the covalent linker is from about 20 to 54 Å in length. In embodiments, the covalent linker is from about 24 to 54 Ain length. In embodiments, the covalent linker is from about 28 to 54 Å in length. In embodiments, the covalent linker is from about 5 to 50 Å in length. In embodiments, the covalent linker is from about 5 to 46 Å in length. In embodiments, the covalent linker is from about 5 to 42 Ain length. In embodiments, the covalent linker is from about 5 to 38 Ain length. In embodiments, the covalent linker is from about 5 to 34 Å in length. In embodiments, the covalent linker is from about 5 to 30 Ain length. In embodiments, the covalent linker is from about 5 to 26 Ain length. In embodiments, the covalent linker is from about 5 to 22 Ain length. In embodiments, the covalent linker is from about 5 to 39 Ain length. In embodiments, the covalent linker is from about 7 to 37 Å in length. In embodiments, the covalent linker is from about 9 to 35 Å in length. In embodiments, the covalent linker is from about 11 to 33 Ain length. In embodiments, the covalent linker is from about 13 to 31 Ain length. In embodiments, the covalent linker is from about 15 to 29 Ain length. In embodiments, the covalent linker is from about 15 to 25 Å in length. In embodiments, the covalent linker is from about 15 to 23 Å in length. In embodiments, the covalent linker is at least or about 32 Å in length (e.g., at least or about 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Ain length). In embodiments, the covalent linker is at least or about the length of 27 methylene groups. In embodiments, the covalent linker is from about 32 to 54 Å in length. In embodiments, the covalent linker is from about 33 to 53 Ain length. In embodiments, the covalent linker is from about 34 to 52 Ain length. In embodiments, the covalent linker is from about 35 to 51 Å in length. In embodiments, the covalent linker is from about 36 to 50 Å in length. In embodiments, the covalent linker is from about 37 to 49 Å in length. In embodiments, the covalent linker is from about 38 to 48 Å in length. In embodiments, the covalent linker is from about 39 to 47 Ain length. In embodiments, the covalent linker is from about 40 to 46 Å in length. In embodiments, the covalent linker is from about 41 to 45 Å in length. In embodiments, the covalent linker is from about 42 to 44 Å in length. In embodiments, the covalent linker is from about 32 to 52 Å in length. In embodiments, the covalent linker is from about 32 to 50 Å in length. In embodiments, the covalent linker is from about 32 to 48 Å in length. In embodiments, the covalent linker is from about 32 to 46 Å in length. In embodiments, the covalent linker is from about 32 to 44 Å in length. In embodiments, the covalent linker is from about 32 to 42 Å in length. In embodiments, the covalent linker is from about 32 to 40 Å in length. In embodiments, the covalent linker is from about 32 to 38 Å in length. In embodiments, the covalent linker is from about 32 to 36 Å in length. In embodiments, the covalent linker is from about 34 to 54 Å in length. In embodiments, the covalent linker is from about 36 to 54 Å in length. In embodiments, the covalent linker is from about 38 to 54 Ain length. In embodiments, the covalent linker is from about 40 to 54 Å in length. In embodiments, the covalent linker is from about 42 to 54 Å in length. In embodiments, the covalent linker is from about 44 to 54 Ain length. In embodiments, the covalent linker is from about 46 to 54 Å in length. In embodiments, the covalent linker is from about 48 to 54 Ain length. In embodiments, the covalent linker is from about 50 to 54 Å in length.

The specified length of a linker is the through space distance between the ends of the linker (i.e., the ends or termini that are connected to the two parts of the molecule connected by the linker) wherein the length of the linker is measured when the linker is fully extended and wherein the linker termini are the furthest apart they may naturally exist in solution (i.e., the longest distance between the ends of the linker wherein the linker adopts allowable conformations, bond lengths, and bond angles following the principles of Chemistry), (e.g., without adopting non-natural bond lengths, non-allowed or non-preferred bond angles, or high energy non-preferred or non-natural interactions of different components of the linker). In embodiments, the linker length is measured when included in a compound as described herein (e.g., aspect, embodiment, example, figures, table, claim). It will be understood that a linker may adopt a through space distance that is less than the fully extended conformation used to define the linker length.

In embodiments, the linker is a hydrolysable linker (e.g., in solution). In embodiments, the linker is a non-hydrolysable linker (e.g., in solution). In embodiments, the linker may be cleaved by an enzyme (e.g., hydrolase, protease, cytochrome). In embodiments, the linker is not cleavable by an enzyme (e.g., under normal cellular conditions). In embodiments, the linker is a polyethylene glycol linker. In embodiments, the linker is hydrophilic. In embodiments, the linker is hydrophobic. In embodiments, the linker includes a disulfide bond. In embodiments, the linker includes a hydrazone bond. In embodiments, the linker includes an ester. In embodiments, the linker includes a sulfonyl. In embodiments, the linker includes a thioether. In embodiments, the linker includes a phosphinate. In embodiments, the linker includes an alkyloxime bond. In embodiments, the linker includes one or more amino acids. In embodiments, the linker consists of amino acids. In embodiments, the linker includes an amino acid analog. In embodiments, the linker includes an amino acid mimetic. In embodiments, the linker is a linker known in the art for use in linking antibodies to agents (e.g., antibody drug conjugates). In embodiments, the linker is a linker as described in Bioconjugate Techniques (Second Edition) by Greg T. Hermanson (2008), which is herein incorporated by referenced in its entirety for all purposes. In embodiments, the linker is a linker as described in Flygare J A, Pillow T H, Aristoff P., Antibody-drug conjugates for the treatment of cancer. Chemical Biology and Drug Design. 2013 January; 81(1):113-21, which is herein incorporated by referenced in its entirety for all purposes. In embodiments, the linker is a linker as described in Drachman J G, Senter P D., Antibody-drug conjugates: the chemistry behind empowering antibodies to fight cancer. Hematology Am Soc Hematol Educ Program. 2013; 2013:306-10, which is herein incorporated by referenced in its entirety for all purposes.

In embodiments, the compound has the formula:

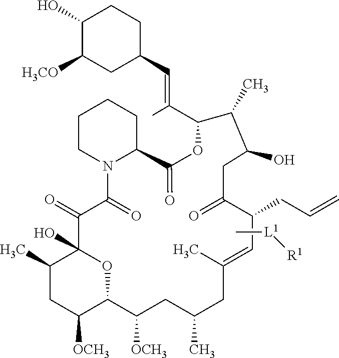

wherein L¹ is as described herein and may be bonded to any atom in the ring (L¹ is a floating substituent) and R¹ is as described herein.

In embodiments, the compound has the formula:

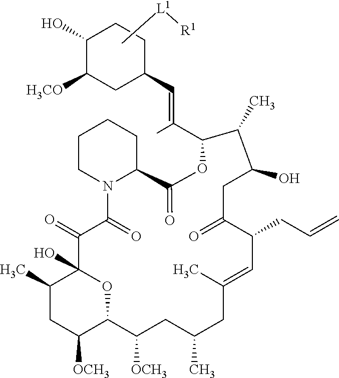

wherein L¹ is as described herein and may be bonded to any atom in the ring (L¹ is a floating substituent) and R¹ is as described herein.

In embodiments, the compound has the formula:

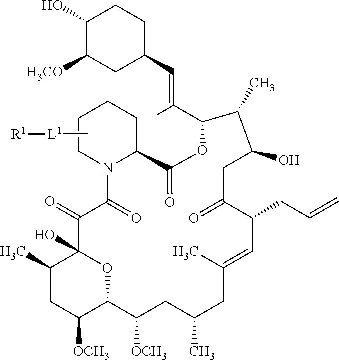

wherein L¹ is as described herein and may be bonded to any atom in the ring (L¹ is a floating substituent) and R¹ is as described herein.

In embodiments, the compound has the formula:

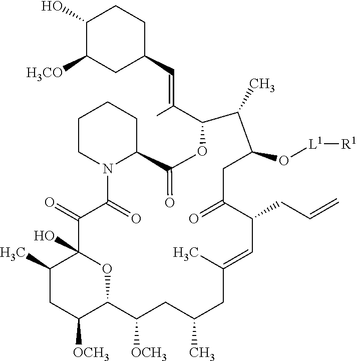

wherein L¹ is as described herein and R¹ is as described herein.

In embodiments, the compound has the formula:

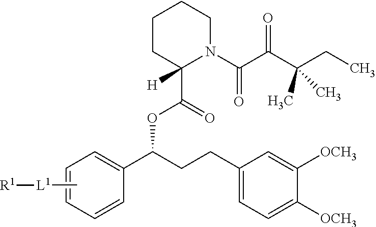

wherein L¹ is as described herein and may be bonded to any atom in the ring (L¹ is a floating substituent) and R¹ is as described herein.

In embodiments, the compound has the formula:

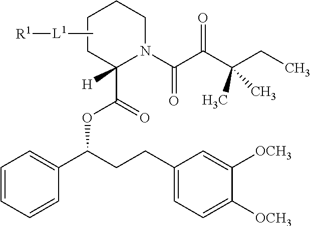

wherein L¹ is as described herein and may be bonded to any atom in the ring (L¹ is a floating substituent) and R¹ is as described herein.

In embodiments, the compound has the formula:

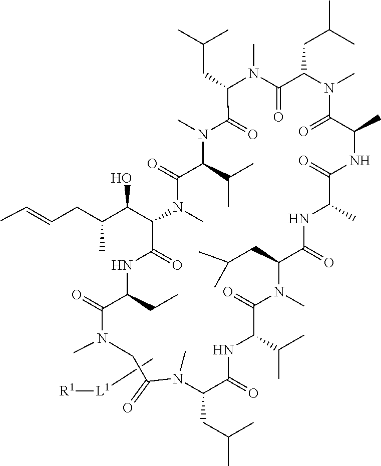

wherein L¹ is as described herein and may be bonded to any atom in the ring (L¹ is a floating substituent) and R¹ is as described herein.

In embodiments, the compound has the formula:

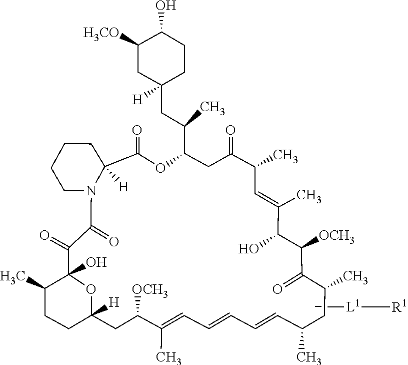

wherein L¹ is as described herein and may be bonded to any atom in the ring (L¹ is a floating substituent) and R¹ is as described herein.

In embodiments, the compound has the formula:

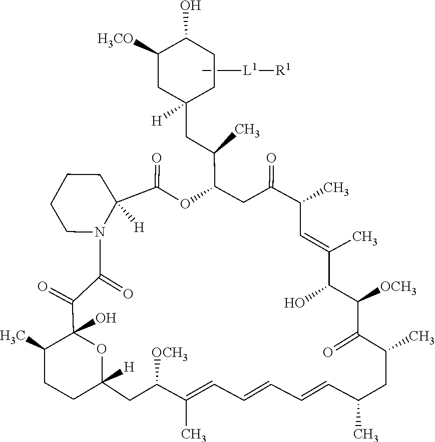

wherein L¹ is as described herein and may be bonded to any atom in the ring (L¹ is a floating substituent) and R¹ is as described herein.

In embodiments, the compound has the formula:

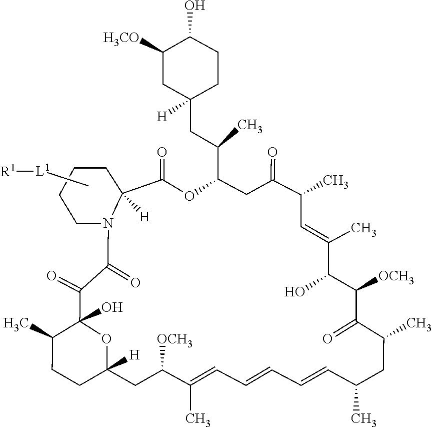

wherein L¹ is as described herein and may be bonded to any atom in the ring (L¹ is a floating substituent) and R¹ is as described herein.

In embodiments, the compound has the formula:

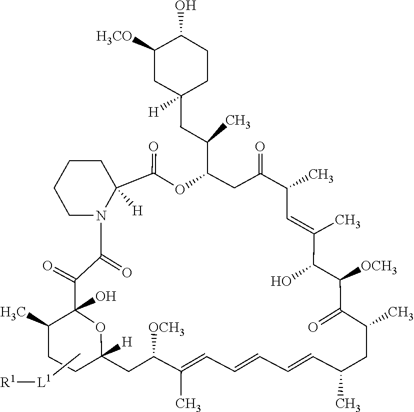

wherein L¹ is as described herein and may be bonded to any atom in the ring (L¹ is a floating substituent) and R¹ is as described herein.

In embodiments, the compound has the formula:

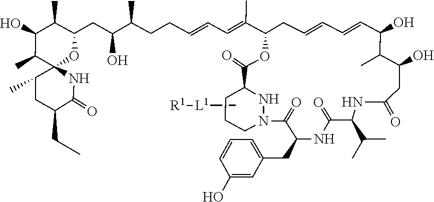

wherein L¹ is as described herein and may be bonded to any atom in the ring (L¹ is a floating substituent) and R¹ is as described herein.

In embodiments, the compound has the formula:

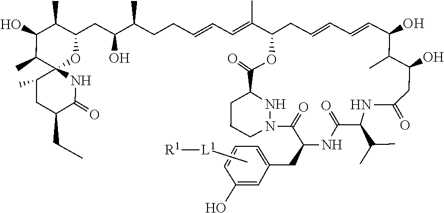

wherein L¹ is as described herein and may be bonded to any atom in the ring (L¹ is a floating substituent) and R¹ is as described herein.

In embodiments, the compound has the formula:

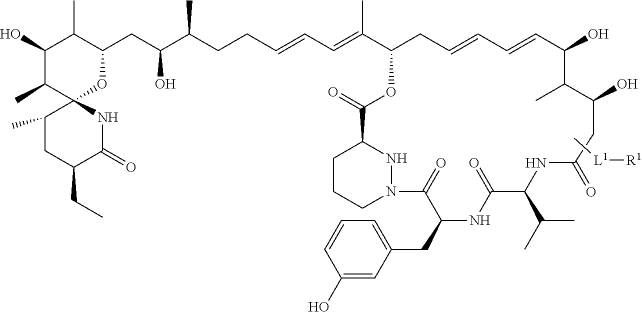

wherein L¹ is as described herein and may be bonded to any atom in the ring (L¹ is a floating substituent) and R¹ is as described herein.

In embodiments, R¹ is a kinase inhibitor moiety In embodiments, R¹ is a pseudokinase inhibitor moiety. In embodiments, R¹ is a GTPase inhibitor moiety. In embodiments, R¹ is a histone-modifying enzyme inhibitor moiety. In embodiments, R¹ is a monovalent anti-viral agent.

In embodiments, R¹ is a kinase inhibitor moiety. In embodiments, R¹ is a protein kinase inhibitor moiety, a lipid kinase inhibitor moiety, or a carbohydrate kinase inhibitor moiety. In embodiments, R¹ is a cyclin dependent kinase inhibitor moiety or a mitogen-activated protein kinase inhibitor moiety. In embodiments, R¹ is a phosphatidylinositol kinase inhibitor moiety or a sphingosine kinase inhibitor moiety. In embodiments, R¹ is a nucleoside-phosphate kinase inhibitor moiety or a nucleoside-diphosphate kinase inhibitor moiety. In embodiments, R¹ is a thymidine kinase inhibitor moiety or a riboflavin kinase inhibitor moiety.

In embodiments, R¹ is a protein kinase inhibitor moiety. In embodiments, R¹ is an AGC kinase inhibitor moiety, a CAM kinase inhibitor moiety, a CK1 kinase inhibitor moiety, a CMGC kinase inhibitor moiety, a STE kinase inhibitor moiety, a TK kinase inhibitor moiety or a TKL kinase inhibitor moiety. In embodiments, R¹ is PKA kinase inhibitor moiety, a PCK kinase inhibitor moiety, or a PKG kinase inhibitor moiety. In embodiments, R¹ is CDK kinase inhibitor moiety, a MAPK kinase inhibitor moiety, a GSK3 kinase inhibitor moiety, or a CLK kinase inhibitor moiety.

In embodiments, R¹ is a serine/threonine-specific protein kinase inhibitor moiety, a tyrosine-specific protein kinase inhibitor moiety, or a histidine-specific protein kinase inhibitor moiety. In embodiments, R¹ is a MAP4K inhibitor moiety. In embodiments, R¹ is a MAP3K inhibitor moiety.

In embodiments, R¹ is a serine/threonine-specific protein kinase inhibitor moiety. In embodiments, R¹ is a CK2 kinase inhibitor moiety, a protein kinase A inhibitor, a protein kinase C inhibitor, a Mos kinase inhibitor moiety, a Raf kinase inhibitor moiety, a mitogen-activated protein kinase (MAPK) inhibitor, a Ca2+/calmodulin-dependent (CaM) protein kinase inhibitor moiety, a phosphorylase kinase inhibitor moiety, a protein kinase B (AKT) inhibitor, or a leucine-rich repeat kinase (LRRK) inhibitor. In embodiments, R¹ is a Raf kinase inhibitor moiety. In embodiments, R¹ is a leucine-rich repeat kinase (LRRK) inhibitor. In embodiments, R¹ is a DLK inhibitor moiety. In embodiments, R¹ is a MAP3K12 inhibitor moiety. In embodiments, R¹ is a HGK inhibitor moiety.

In embodiments, R¹ is a tyrosine-specific protein kinase inhibitor moiety. In embodiments, R¹ is a receptor tyrosine kinase inhibitor moiety or a non-receptor tyrosine kinase inhibitor moiety.

In embodiments, R¹ is a receptor tyrosine kinase inhibitor moiety. In embodiments, R¹ is a platelet-derived growth factor (PDGFR) kinase inhibitor moiety, an epidermal growth factor (EGFR) kinase inhibitor moiety, a HER2 kinase inhibitor moiety, an insulin receptor kinase inhibitor moiety, an insulin-like growth factor 1 (IGF1R) kinase inhibitor moiety, a vascular endothelial growth factor (VEGFR) inhibitor, a stem cell factor (SCF) kinase inhibitor moiety, a fibroblast growth factor (FGF) kinase inhibitor moiety, a colon carcinoma kinase 4 (CCK4) kinase inhibitor moiety, a NGF kinase inhibitor moiety, a c-KIT kinase inhibitor moiety, or a hepatocyte growth factor receptor (HGFR) kinase inhibitor moiety. In embodiments, R¹ is a platelet-derived growth factor (PDGFR) kinase inhibitor moiety. In embodiments, R¹ is an epidermal growth factor (EGFR) kinase inhibitor moiety. In embodiments, R¹ is a vascular endothelial growth factor (VEGFR) kinase inhibitor moiety. In embodiments, R¹ is a c-KIT kinase inhibitor moiety.

In embodiments, R¹ is a non-receptor tyrosine kinase inhibitor moiety. In embodiments, R¹ is an Abl kinase inhibitor moiety, an Ack kinase inhibitor moiety, a Csk kinase inhibitor moiety, a Fak kinase inhibitor moiety, a Fes kinase inhibitor moiety, a Frk kinase inhibitor moiety, a Jak kinase inhibitor moiety, a Src kinase inhibitor moiety, a Syk kinase inhibitor moiety, or a Tec kinase inhibitor moiety. In embodiments, R¹ is a Src kinase inhibitor moiety. In embodiments, R¹ is a PERK kinase inhibitor moiety. In embodiments, R¹ is a GSK3 kinase inhibitor moiety. In embodiments, R¹ is a p38α MAPK kinase inhibitor moiety.

In embodiments, R¹ is a pseudokinase inhibitor moiety (e.g., a HER3 inhibitor moiety).

In embodiments, R¹ is a GTPase inhibitor moiety (e.g., K-Ras inhibitor, K-RAs4A inhibitor, K-Ras4B inhibitor).

In embodiments, R¹ is a histone modifying enzyme inhibitor moiety (e.g., SET3D).

In embodiments, R¹ is a monovalent an anti-cancer agent (e.g., as described herein). In embodiments, R¹ is a monovalent a chemotherapeutic agent (e.g., as described herein). In embodiments, R¹ is a monovalent anti-neurodegenerative disease agent (e.g., as described herein). In embodiments, R¹ is a monovalent anti-viral agent (e.g., as described herein).

In embodiments, R¹ is a monovalent anti-viral agent. In embodiments, R¹ is not a monovalent anti-viral agent. In embodiments, R¹ is not an anti-HIV agent. In embodiments, R¹ is not an HIV inhibitor. In embodiments, R¹ is not an HIV protease inhibitor. In embodiments, R¹ is not a viral protease inhibitor. In embodiments, R¹ is not a monovalent HIV inhibitor. In embodiments, R¹ is not a monovalent HIV protease inhibitor. In embodiments, R¹ is not a monovalent viral protease inhibitor. In embodiments, R¹ is not a monovalent amprenavir, or analog thereof. In embodiments, R¹ is not a monovalent amprenavir. In embodiments, R¹ is not a monovalent 4-methoxy amprenavir, or analog thereof. In embodiments, R¹ is not a monovalent 4-methoxy amprenavir.

In embodiments, R¹ is not an amyloid R aggregation inhibitor. In embodiments, R¹ is not a monovalent congo red, or analog thereof. In embodiments, R¹ is not a monovalent thioflavin T, or analog thereof. In embodiments, R¹ is not a monovalent curcumin, or analog thereof.

In embodiments, the monovalent Src kinase inhibitor is a monovalent dasatinib, monovalent saracatinib, monovalent bosutinib, or monovalent KXO1, or an analog thereof.

In embodiments, the monovalent Raf, VEGFR, PDGFR, or c-Kit inhibitor is a monovalent sorafenib, monovalent imatinib, monovalent nilotinib, monovalent sunitinib, monovalent dasatinib, monovalent pazopanib, monovalent vandetanib, monovalent axitinib, monovalent levatinib, monovalent regorafenib, or an analog thereof.

In embodiments, the monovalent EGFR inhibitor is a monovalent lapatinib, monovalent erlotinib, monovalent gefitinib, monovalent vandetanib, monovalent osimertinib, monovalent regorafenib, monovalent AZD 9291, monovalent AG 1478, monovalent dacomitinib, monovalent afatinib, monovalent WZ 4002, monovalent CO-1686, monovalent neratinib, monovalent canertinib, monovalent AC-480, monovalent AZD 8931, monovalent AST 1306, or monovalent EKB 569, or an analog thereof.

In embodiments, the monovalent LRRK2 inhibitor is a monovalent staurosporine, monovalent K-252a, monovalent K-252b, monovalent G66976, monovalent GF109203X, monovalent Ro31-8220, monovalent 5-iodotubericidin, monovalent sorafenib, monovalent GW5074 (Raf-1 kinase inhibitor), monovalent indirubin-3′-monooxime, monovalent sunitinib, monovalent H-1152, monovalent Compound 4, monovalent Y-27632, monovalent SP600125, monovalent damnacanthal, monovalent LDN-73794, monovalent LDN-22684, monovalent CZC-25146, monovalent CZC-54252, monovalent LRRK2-IN-1, monovalent HG-10-102-1, monovalent GSK2578215A, monovalent JH-II-127, monovalent GNE-0877, monovalent GNE-9605, monovalent PF-06447475, monovalent MLi-2, or monovalent DNL201, or analog thereof.

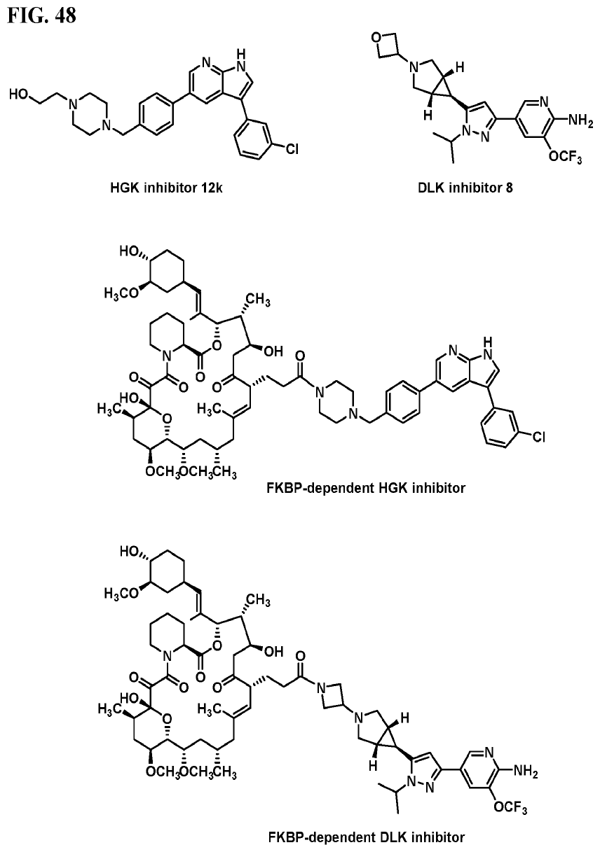

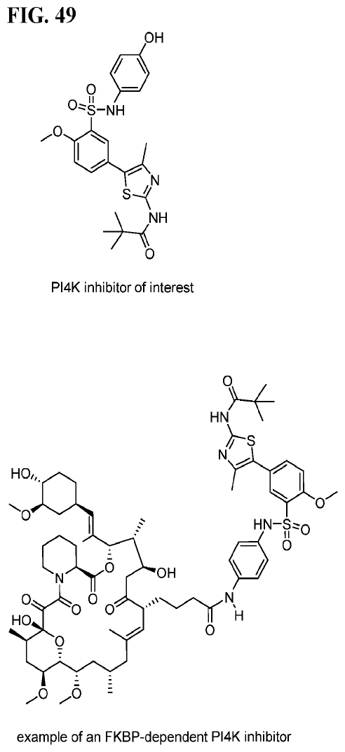

In embodiments, the monovalent HGK inhibitor is a monovalent compound 12k (as shown in FIG. 48 and as described in Cell Chemical Biology, 2019, 26, 1703-1715, which is herein incorporated by reference in its entirety for all purposes). In embodiments, the monovalent HGK inhibitor is a monovalent URMC-099, a monovalent PF06260933, or a monovalent GNE-495.

In embodiments, the monovalent DLK inhibitor is a monovalent DLK inhibitor 8 (as shown in FIG. 48 and as described in J. Med. Chem., 2018, 61, 8078-8087, which is herein incorporated by reference in its entirety for all purposes). In embodiments, the monovalent DLK inhibitor is a monovalent sunitinib, monovalent tozasertib, monovalent GNE-8505, or monovalent GNE-3511.

In embodiments, the monovalent KRAS inhibitor is a monovalent KRAS G12C inhibitor, monovalent KRAS M72C inhibitor, monovalent AMG510, monovalent MRTX849, monovalent ARS-1620, or analog thereof.

In embodiments, the monovalent PI4KIIIβ inhibitor is as described in J. Med. Chem., 2016, 59 (5), 1830-1839. In embodiments, the monovalent PI4KIIIβ inhibitor is a monovalent PI4K inhibitor as shown in FIG. 48 (and as described in J Med. Chem., 2016, 59 (5), 1830-1839, which is herein incorporated by reference in its entirety for all purposes). In embodiments, the monovalent PI4KIIIβ inhibitor is

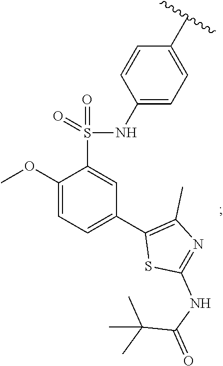 [SMILES: *c1ccc(NS(=O)(=O)c2cc(-c3sc(NC(=O)C(C)(C)C)nc3C)ccc2OC)cc1]

or an analog thereof.

In embodiments, R¹ is

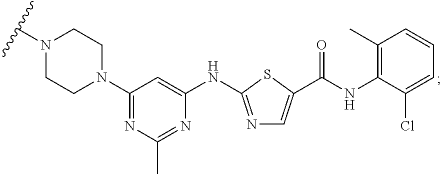 [SMILES: *N1CCN(c2cc(Nc3ncc(C(=O)Nc4c(C)cccc4Cl)s3)nc(C)n2)CC1]

or an analog thereof.

In embodiments, R¹ is

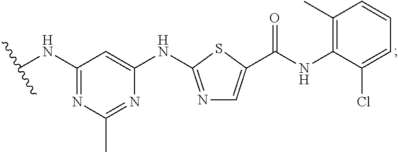 [SMILES: *Nc1cc(Nc2ncc(C(=O)Nc3c(C)cccc3Cl)s2)nc(C)n1]

or an analog thereof.

In embodiments, R¹ is

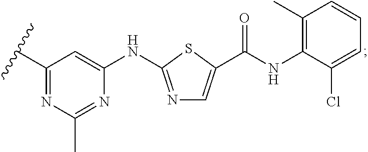 [SMILES: *c1cc(Nc2ncc(C(=O)Nc3c(C)cccc3Cl)s2)nc(C)n1]

or an analog thereof.

In embodiments, R¹ is

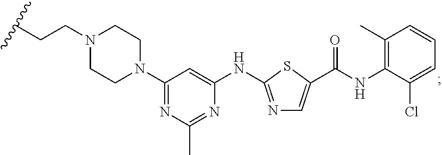 [SMILES: *CCN1CCN(c2cc(Nc3ncc(C(=O)Nc4c(C)cccc4Cl)s3)nc(C)n2)CC1]

or an analog thereof.

In embodiments, R¹ is

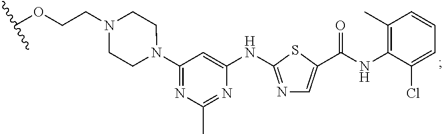 [SMILES: *OCCN1CCN(c2cc(Nc3ncc(C(=O)Nc4c(C)cccc4Cl)s3)nc(C)n2)CC1]

or an analog thereof.

In embodiments, R¹ is

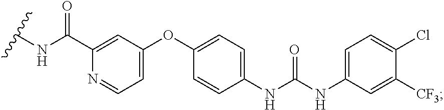 [SMILES: *NC(=O)c1cc(Oc2ccc(NC(=O)Nc3ccc(Cl)c(C(F)(F)F)c3)cc2)ccn1]

or an analog thereof.

In embodiments, R¹ is

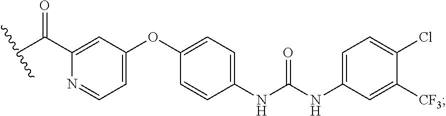 [SMILES: *C(=O)c1cc(Oc2ccc(NC(=O)Nc3ccc(Cl)c(C(F)(F)F)c3)cc2)ccn1]

or an analog thereof.

In embodiments, R¹ is

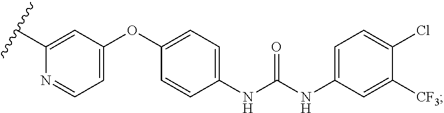 [SMILES: *c1cc(Oc2ccc(NC(=O)Nc3ccc(Cl)c(C(F)(F)F)c3)cc2)ccn1]

or an analog thereof.

In embodiments, R¹ is

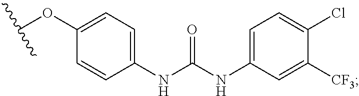 [SMILES: *Oc1ccc(NC(=O)Nc2ccc(Cl)c(C(F)(F)F)c2)cc1]

or an analog thereof.

In embodiments, R¹ is

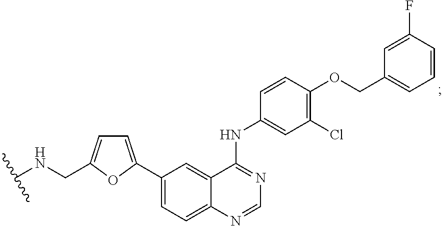 [SMILES: *NCc1ccc(-c2ccc3ncnc(Nc4ccc(OCc5cccc(F)c5)c(Cl)c4)c3c2)o1]

or an analog thereof.

In embodiments, R¹ is

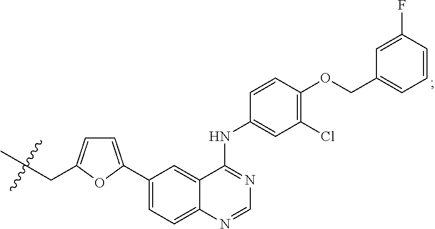 [SMILES: *Cc1ccc(-c2ccc3ncnc(Nc4ccc(OCc5cccc(F)c5)c(Cl)c4)c3c2)o1]

or an analog thereof.

In embodiments, R¹ is

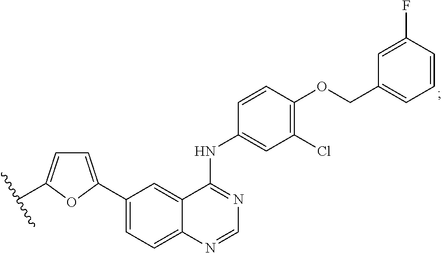 [SMILES: *c1ccc(-c2ccc3ncnc(Nc4ccc(OCc5cccc(F)c5)c(Cl)c4)c3c2)o1]

or an analog thereof.

In embodiments, R¹ is

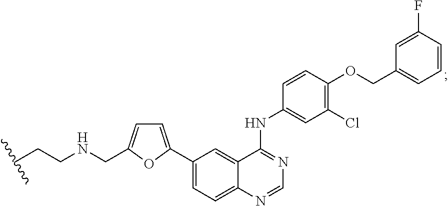 [SMILES: *CCNCc1ccc(-c2ccc3ncnc(Nc4ccc(OCc5cccc(F)c5)c(Cl)c4)c3c2)o1]

or an analog thereof.

In embodiments, R¹ is

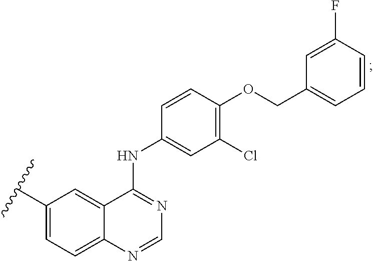 [SMILES: *c1ccc2ncnc(Nc3ccc(OCc4cccc(F)c4)c(Cl)c3)c2c1]

or an analog thereof.

In embodiments, R¹ is

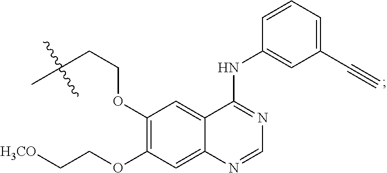 [SMILES: *CCOc1cc2c(Nc3cccc(C#C)c3)ncnc2cc1OCCOC]

or an analog thereof.

In embodiments, R¹ is

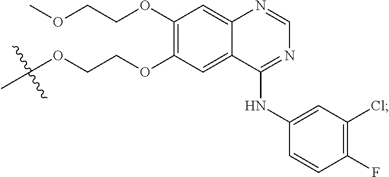 [SMILES: *OCCOc1cc2c(Nc3ccc(F)c(Cl)c3)ncnc2cc1OCCOC]

or an analog thereof.

In embodiments, R¹ is

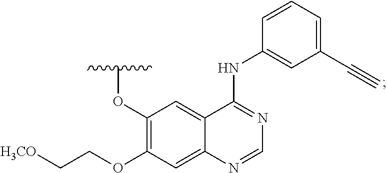 [SMILES: *Oc1cc2c(Nc3cccc(C#C)c3)ncnc2cc1OCCOC]

or an analog thereof.

In embodiments, R¹ is

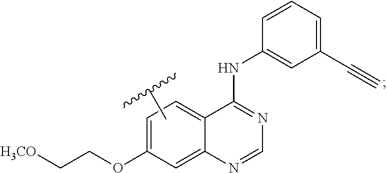

or an analog thereof.

In embodiments, R¹ is

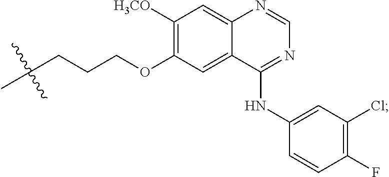 [SMILES: *CCCOc1cc2c(Nc3ccc(F)c(Cl)c3)ncnc2cc1OC]

or an analog thereof.

In embodiments, R¹ is

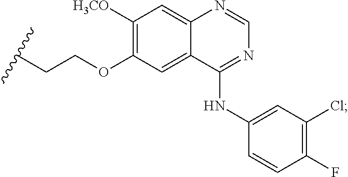 [SMILES: *CCOc1cc2c(Nc3ccc(F)c(Cl)c3)ncnc2cc1OC]

or an analog thereof.

In embodiments, R¹ is

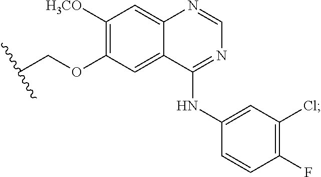 [SMILES: *COc1cc2c(Nc3ccc(F)c(Cl)c3)ncnc2cc1OC]

or an analog thereof.

In embodiments, R¹ is

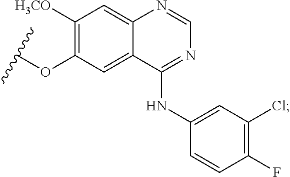 [SMILES: *Oc1cc2c(Nc3ccc(F)c(Cl)c3)ncnc2cc1OC]

or an analog thereof.

In embodiments, R¹ is

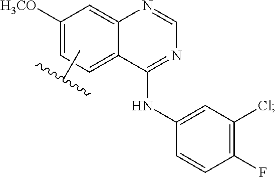

or an analog thereof.

In embodiments, R¹ is

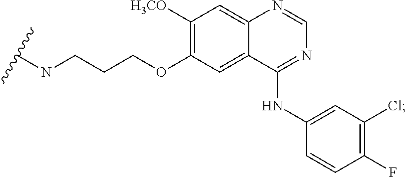 [SMILES: *NCCCOc1cc2c(Nc3ccc(F)c(Cl)c3)ncnc2cc1OC]

or an analog thereof.

In embodiments, R¹ is

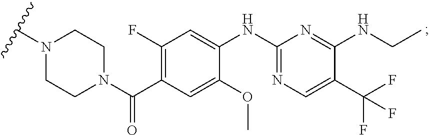 [SMILES: *N1CCN(C(=O)c2cc(OC)c(Nc3ncc(C(F)(F)F)c(NCC)n3)cc2F)CC1]

or an analog thereof.

In embodiments, R¹

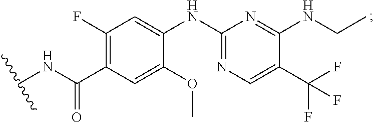 [SMILES: *NC(=O)c1cc(OC)c(Nc2ncc(C(F)(F)F)c(NCC)n2)cc1F]

or an analog thereof.

In embodiments, R¹

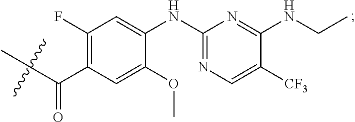 [SMILES: *C(=O)c1cc(OC)c(Nc2ncc(C(F)(F)F)c(NCC)n2)cc1F]

or an analog thereof.

In embodiments, R¹ is

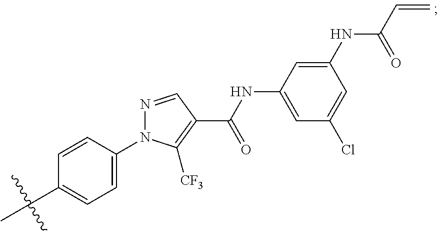 [SMILES: *c1ccc(-n2ncc(C(=O)Nc3cc(Cl)cc(NC(=O)C=C)c3)c2C(F)(F)F)cc1]

or an analog thereof.

In embodiments, R¹ is

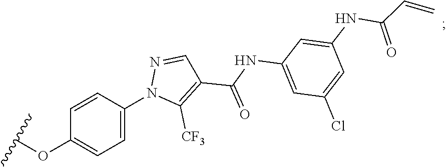 [SMILES: *Oc1ccc(-n2ncc(C(=O)Nc3cc(Cl)cc(NC(=O)C=C)c3)c2C(F)(F)F)cc1]

or an analog thereof.

In embodiments, R¹ is

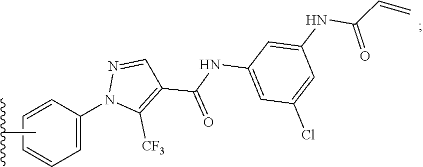

or an analog thereof.

In embodiments, R¹ is

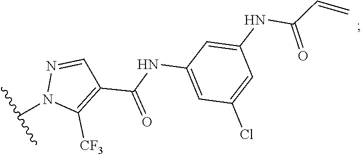 [SMILES: *n1ncc(C(=O)Nc2cc(Cl)cc(NC(=O)C=C)c2)c1C(F)(F)F]

or an analog thereof.

In embodiments, R¹ is

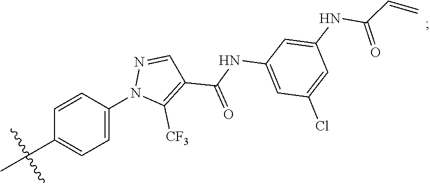 [SMILES: *c1ccc(-n2ncc(C(=O)Nc3cc(Cl)cc(NC(=O)C=C)c3)c2C(F)(F)F)cc1]

or an analog thereof.

In embodiments, R¹ is

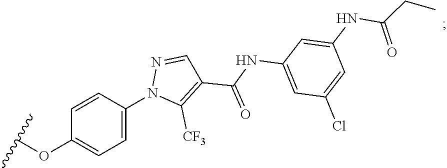 [SMILES: *Oc1ccc(-n2ncc(C(=O)Nc3cc(Cl)cc(NC(=O)CC)c3)c2C(F)(F)F)cc1]

or an analog thereof.

In embodiments, R¹ is

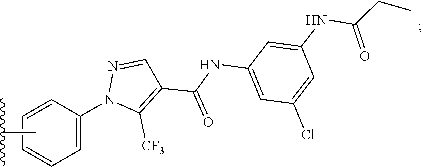

or an analog thereof.

In embodiments, R¹ is

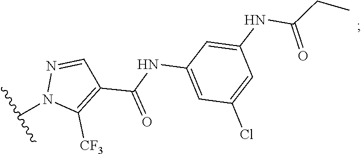 [SMILES: *n1ncc(C(=O)Nc2cc(Cl)cc(NC(=O)CC)c2)c1C(F)(F)F]

or an analog thereof.

In embodiments, R¹ is

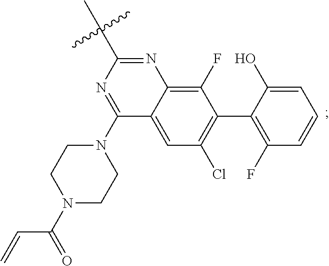 [SMILES: *c1nc(N2CCN(C(=O)C=C)CC2)c2cc(Cl)c(-c3c(O)cccc3F)c(F)c2n1]

or an analog thereof.

In embodiments, R¹ is

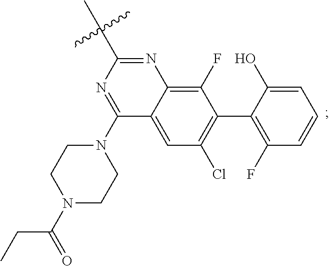 [SMILES: *c1nc(N2CCN(C(=O)CC)CC2)c2cc(Cl)c(-c3c(O)cccc3F)c(F)c2n1]

or an analog thereof.

In embodiments, R¹ is

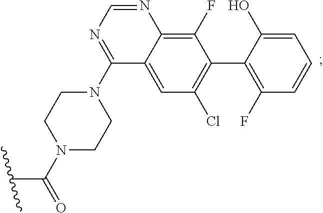 [SMILES: *C(=O)N1CCN(c2ncnc3c(F)c(-c4c(O)cccc4F)c(Cl)cc23)CC1]

or an analog thereof.

In embodiments, R¹ is

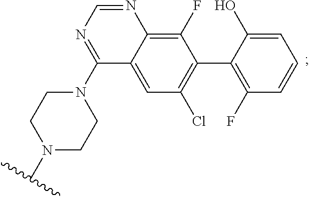 [SMILES: *N1CCN(c2ncnc3c(F)c(-c4c(O)cccc4F)c(Cl)cc23)CC1]

or an analog thereof.

In embodiments, R¹ is

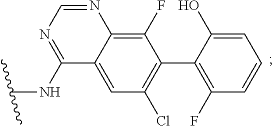 [SMILES: *Nc1ncnc2c(F)c(-c3c(O)cccc3F)c(Cl)cc12]

or an analog thereof.

In embodiments, R¹ is

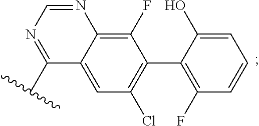 [SMILES: *c1ncnc2c(F)c(-c3c(O)cccc3F)c(Cl)cc12]

or an analog thereof.

In embodiments, R¹ is

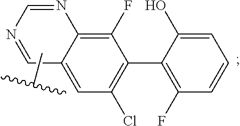

or an analog thereof.

In embodiments, R¹ is

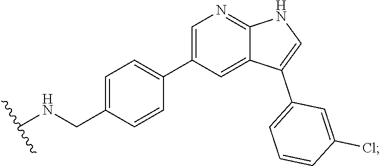 [SMILES: *NCc1ccc(-c2cnc3[nH]cc(-c4cccc(Cl)c4)c3c2)cc1]

or an analog thereof.

In embodiments, R¹ is

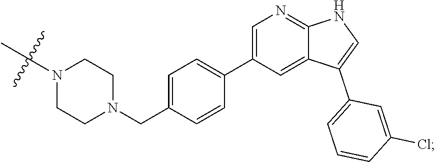 [SMILES: *N1CCN(Cc2ccc(-c3cnc4[nH]cc(-c5cccc(Cl)c5)c4c3)cc2)CC1]

or an analog thereof.

In embodiments, R¹ is

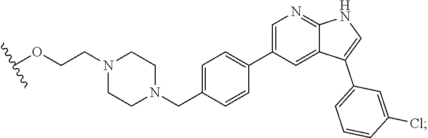 [SMILES: *OCCN1CCN(Cc2ccc(-c3cnc4[nH]cc(-c5cccc(Cl)c5)c4c3)cc2)CC1]

or an analog thereof.

In embodiments, R¹ is

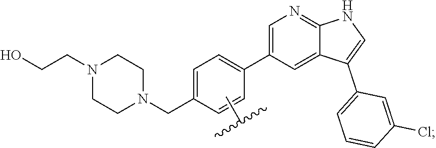

or an analog thereof.

In embodiments, R¹ is

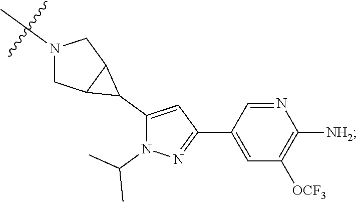 [SMILES: *N1CC2C(C1)C2c1cc(-c2cnc(N)c(OC(F)(F)F)c2)nn1C(C)C]

or an analog thereof.

In embodiments, R¹ is

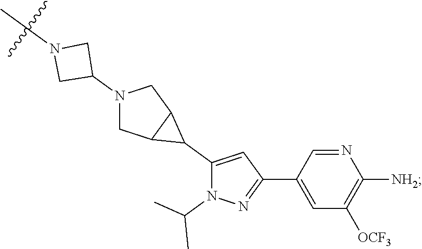 [SMILES: *N1CC(N2CC3C(C2)C3c2cc(-c3cnc(N)c(OC(F)(F)F)c3)nn2C(C)C)C1]

or an analog thereof.

In embodiments, R¹ is

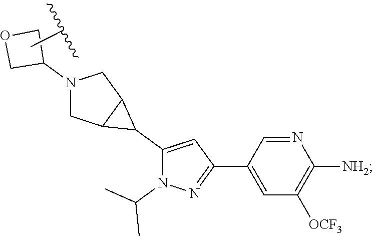

or an analog thereof.

In embodiments, R¹ is

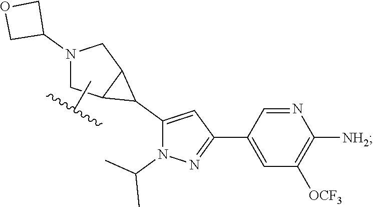

or an analog thereof.

In embodiments, R¹ is

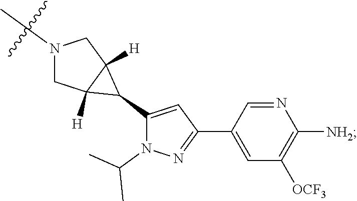 [SMILES: *N1C[C@@H]2[C@H](C1)[C@H]2c1cc(-c2cnc(N)c(OC(F)(F)F)c2)nn1C(C)C]

or an analog thereof.

In embodiments, R¹ is

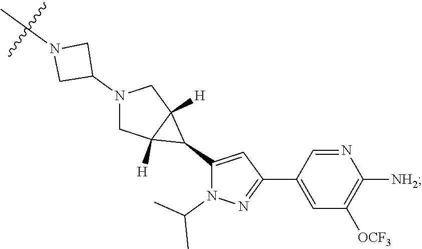 [SMILES: *N1CC(N2C[C@@H]3[C@H](C2)[C@H]3c2cc(-c3cnc(N)c(OC(F)(F)F)c3)nn2C(C)C)C1]

or an analog thereof.

In embodiments, R¹ is

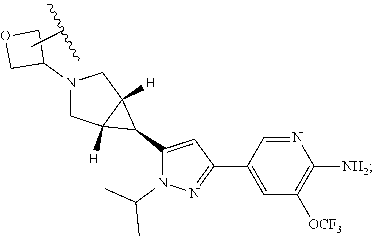

or an analog thereof.

In embodiments, R¹ is

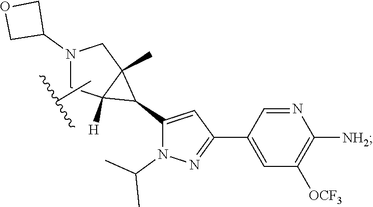

or an analog thereof.

In embodiments, R¹ is

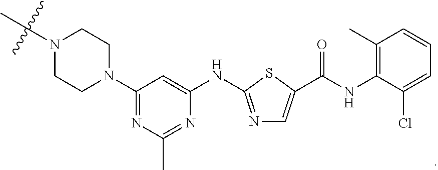 [SMILES: *N1CCN(c2cc(Nc3ncc(C(=O)Nc4c(C)cccc4Cl)s3)nc(C)n2)CC1]

In embodiments, R¹ is

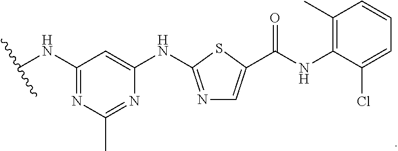 [SMILES: *Nc1cc(Nc2ncc(C(=O)Nc3c(C)cccc3Cl)s2)nc(C)n1]

In embodiments, R¹ is

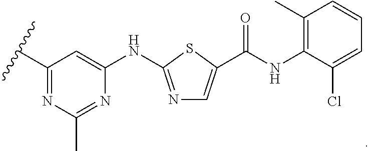 [SMILES: *c1cc(Nc2ncc(C(=O)Nc3c(C)cccc3Cl)s2)nc(C)n1]

In embodiments, R¹ is

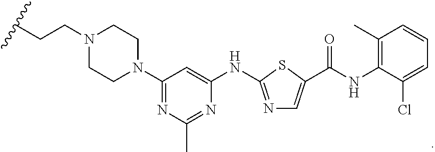 [SMILES: *CCN1CCN(c2cc(Nc3ncc(C(=O)Nc4c(C)cccc4Cl)s3)nc(C)n2)CC1]

In embodiments, R¹ is

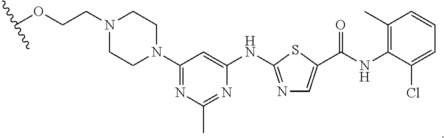 [SMILES: *OCCN1CCN(c2cc(Nc3ncc(C(=O)Nc4c(C)cccc4Cl)s3)nc(C)n2)CC1 ; "."]

In embodiments, R¹ is

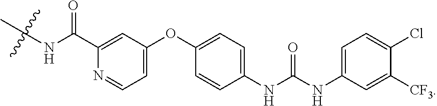 [SMILES: *NC(=O)c1cc(Oc2ccc(NC(=O)Nc3ccc(Cl)c(C(F)(F)F)c3)cc2)ccn1]

In embodiments, R¹ is

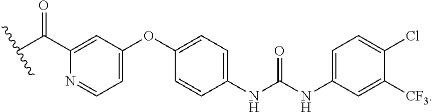 [SMILES: *C(=O)c1cc(Oc2ccc(NC(=O)Nc3ccc(Cl)c(C(F)(F)F)c3)cc2)ccn1]

In embodiments, R¹ is

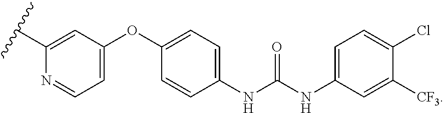 [SMILES: *c1cc(Oc2ccc(NC(=O)Nc3ccc(Cl)c(C(F)(F)F)c3)cc2)ccn1]

In embodiments, R¹ is

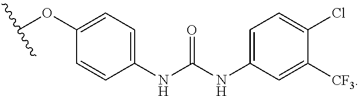 [SMILES: *Oc1ccc(NC(=O)Nc2ccc(Cl)c(C(F)(F)F)c2)cc1]

In embodiments, R¹ is

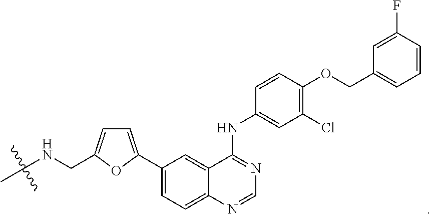 [SMILES: *NCc1ccc(-c2ccc3ncnc(Nc4ccc(OCc5cccc(F)c5)c(Cl)c4)c3c2)o1 ; "."]

In embodiments, R¹ is

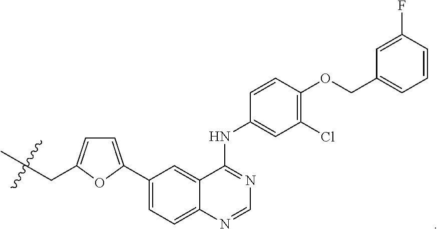 [SMILES: *Cc1ccc(-c2ccc3ncnc(Nc4ccc(OCc5cccc(F)c5)c(Cl)c4)c3c2)o1 ; "."]

In embodiments, R¹ is

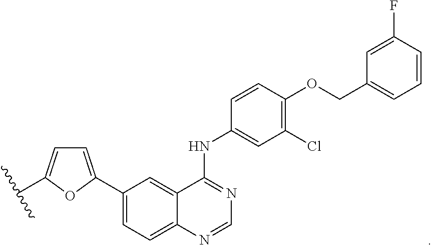 [SMILES: *c1ccc(-c2ccc3ncnc(Nc4ccc(OCc5cccc(F)c5)c(Cl)c4)c3c2)o1 ; "."]

In embodiments, R¹ is

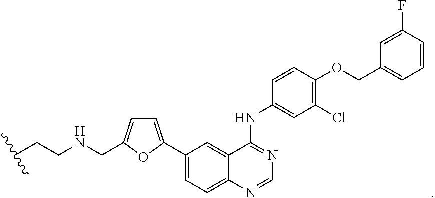 [SMILES: *CCNCc1ccc(-c2ccc3ncnc(Nc4ccc(OCc5cccc(F)c5)c(Cl)c4)c3c2)o1 ; "."]

In embodiments, R¹ is

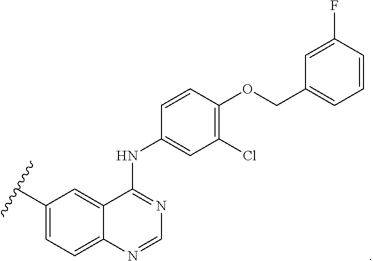 [SMILES: *c1ccc2ncnc(Nc3ccc(OCc4cccc(F)c4)c(Cl)c3)c2c1 ; "."]

In embodiments, R¹ is

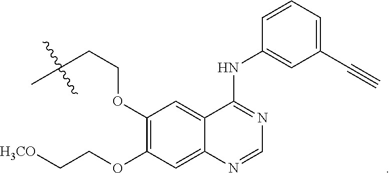 [SMILES: *CCOc1cc2c(Nc3cccc(C#C)c3)ncnc2cc1OCCOC ; "."]

In embodiments, R¹ is

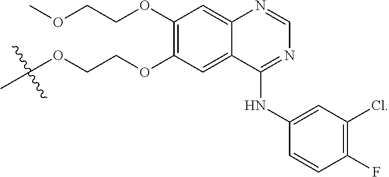 [SMILES: *OCCOc1cc2c(Nc3ccc(F)c(Cl)c3)ncnc2cc1OCCOC]

In embodiments, R¹ is

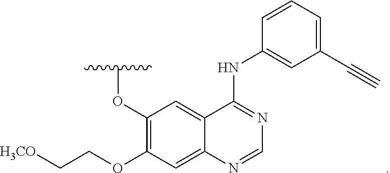 [SMILES: *Oc1cc2c(Nc3cccc(C#C)c3)ncnc2cc1OCCOC ; "."]

In embodiments, R¹ is

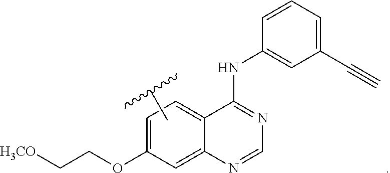

In embodiments, R¹ is

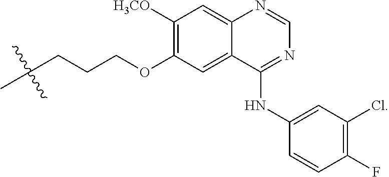 [SMILES: *CCCOc1cc2c(Nc3ccc(F)c(Cl)c3)ncnc2cc1OC]

In embodiments, R¹ is

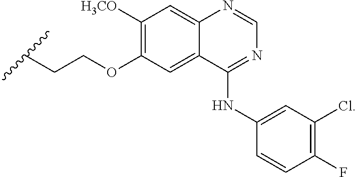 [SMILES: *CCOc1cc2c(Nc3ccc(F)c(Cl)c3)ncnc2cc1OC]

In embodiments, R¹ is

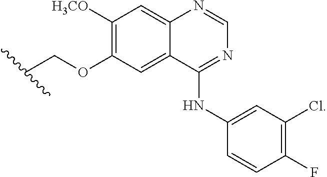 [SMILES: *COc1cc2c(Nc3ccc(F)c(Cl)c3)ncnc2cc1OC]

In embodiments, R¹ is

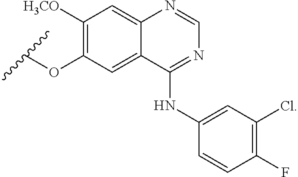 [SMILES: *Oc1cc2c(Nc3ccc(F)c(Cl)c3)ncnc2cc1OC]

In embodiments, R¹ is

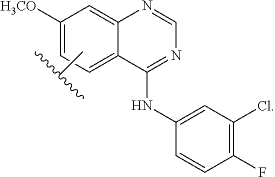

In embodiments, R¹ is

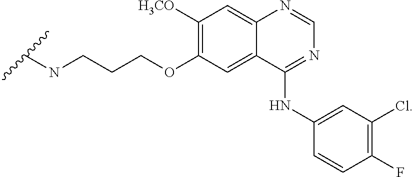 [SMILES: *NCCCOc1cc2c(Nc3ccc(F)c(Cl)c3)ncnc2cc1OC]

In embodiments, R¹ is

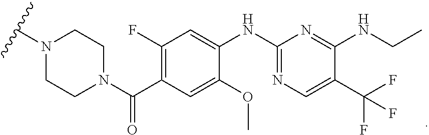 [SMILES: *N1CCN(C(=O)c2cc(OC)c(Nc3ncc(C(F)(F)F)c(NCC)n3)cc2F)CC1]

In embodiments, R¹ is

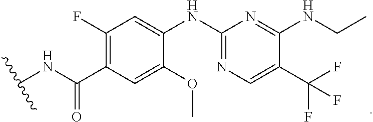 [SMILES: *NC(=O)c1cc(OC)c(Nc2ncc(C(F)(F)F)c(NCC)n2)cc1F]

In embodiments, R¹ is

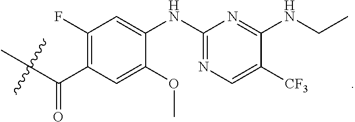 [SMILES: *C(=O)c1cc(OC)c(Nc2ncc(C(F)(F)F)c(NCC)n2)cc1F]

In embodiments, R¹ is

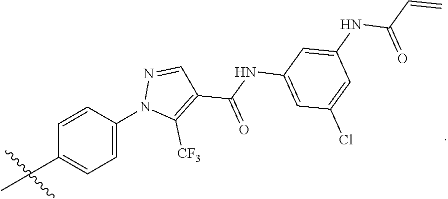 [SMILES: *c1ccc(-n2ncc(C(=O)Nc3cc(Cl)cc(NC(=O)C=C)c3)c2C(F)(F)F)cc1]

In embodiments, R¹ is

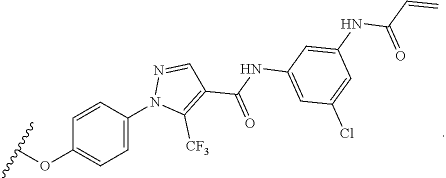 [SMILES: *Oc1ccc(-n2ncc(C(=O)Nc3cc(Cl)cc(NC(=O)C=C)c3)c2C(F)(F)F)cc1]

In embodiments, R¹ is

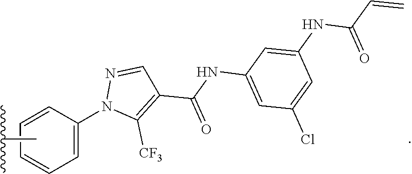

In embodiments, R¹ is

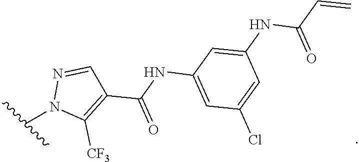 [SMILES: *n1ncc(C(=O)Nc2cc(Cl)cc(NC(=O)C=C)c2)c1C(F)(F)F]

In embodiments, R¹ is

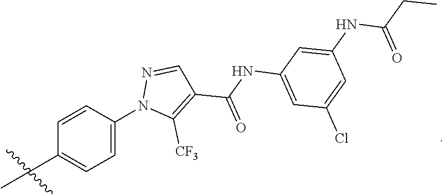 [SMILES: *c1ccc(-n2ncc(C(=O)Nc3cc(Cl)cc(NC(=O)CC)c3)c2C(F)(F)F)cc1]

In embodiments, R¹ is

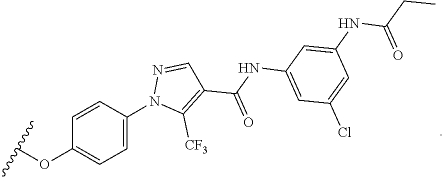 [SMILES: *Oc1ccc(-n2ncc(C(=O)Nc3cc(Cl)cc(NC(=O)CC)c3)c2C(F)(F)F)cc1]

In embodiments, R¹ is

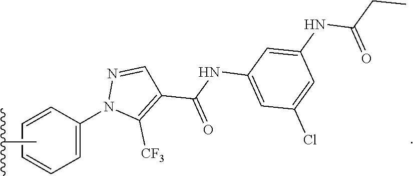

In embodiments, R¹ is

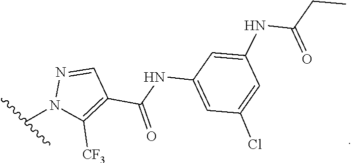 [SMILES: *n1ncc(C(=O)Nc2cc(Cl)cc(NC(=O)CC)c2)c1C(F)(F)F]

In embodiments, R¹ is

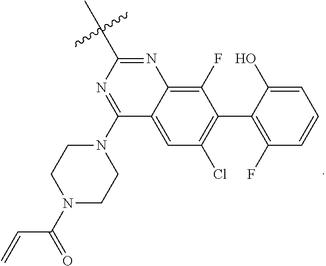 [SMILES: *c1nc(N2CCN(C(=O)C=C)CC2)c2cc(Cl)c(-c3c(O)cccc3F)c(F)c2n1]

In embodiments, R¹ is

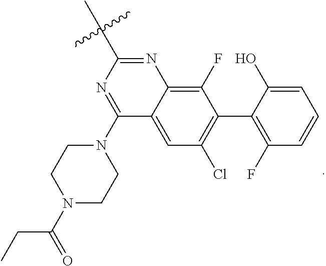 [SMILES: *c1nc(N2CCN(C(=O)CC)CC2)c2cc(Cl)c(-c3c(O)cccc3F)c(F)c2n1]

In embodiments, R¹ is

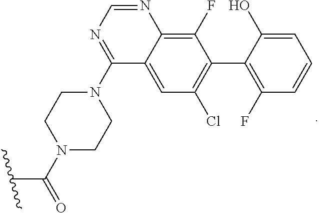 [SMILES: *C(=O)N1CCN(c2ncnc3c(F)c(-c4c(O)cccc4F)c(Cl)cc23)CC1]

In embodiments, R¹ is

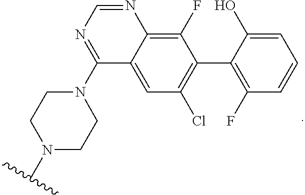 [SMILES: *N1CCN(c2ncnc3c(F)c(-c4c(O)cccc4F)c(Cl)cc23)CC1]

In embodiments, R¹ is

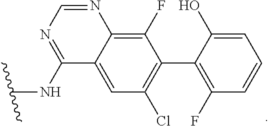 [SMILES: *Nc1ncnc2c(F)c(-c3c(O)cccc3F)c(Cl)cc12]

In embodiments, R¹ is

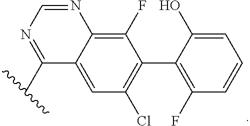 [SMILES: *c1ncnc2c(F)c(-c3c(O)cccc3F)c(Cl)cc12]

In embodiments, R¹ is

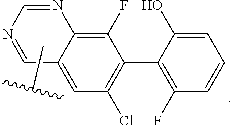

In embodiments, R¹ is

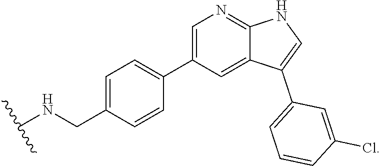 [SMILES: *NCc1ccc(-c2cnc3[nH]cc(-c4cccc(Cl)c4)c3c2)cc1]

In embodiments, R¹

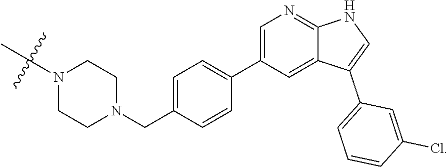 [SMILES: *N1CCN(Cc2ccc(-c3cnc4[nH]cc(-c5cccc(Cl)c5)c4c3)cc2)CC1]

In embodiments, R¹ is

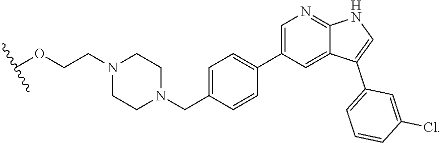 [SMILES: *OCCN1CCN(Cc2ccc(-c3cnc4[nH]cc(-c5cccc(Cl)c5)c4c3)cc2)CC1]

In embodiments, R¹ is

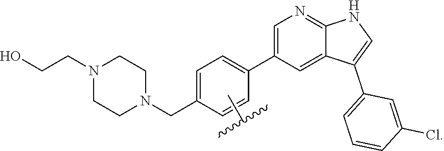

In embodiments, R¹ is

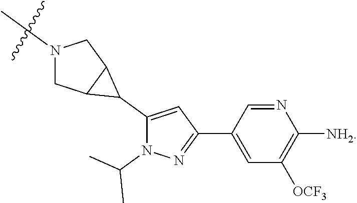 [SMILES: *N1CC2C(C1)C2c1cc(-c2cnc(N)c(OC(F)(F)F)c2)nn1C(C)C]

In embodiments, R¹ is

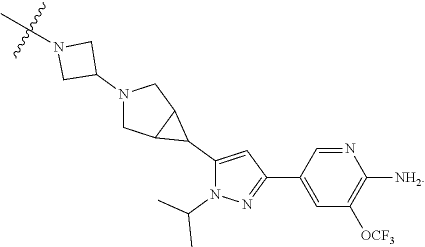 [SMILES: *N1CC(N2CC3C(C2)C3c2cc(-c3cnc(N)c(OC(F)(F)F)c3)nn2C(C)C)C1]

In embodiments, R¹ is

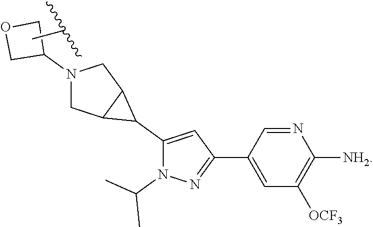

In embodiments, R¹ is

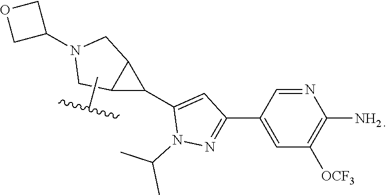

In embodiments, R¹ is

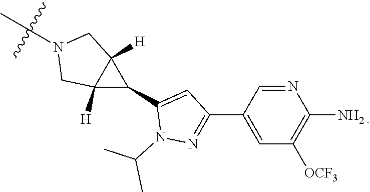 [SMILES: *N1C[C@@H]2[C@H](C1)[C@H]2c1cc(-c2cnc(N)c(OC(F)(F)F)c2)nn1C(C)C]

In embodiments, R¹ is

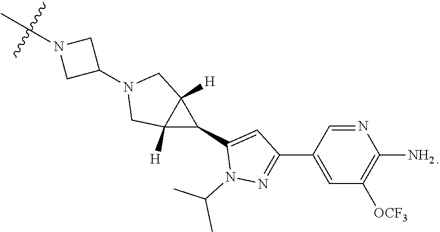 [SMILES: *N1CC(N2C[C@@H]3[C@H](C2)[C@H]3c2cc(-c3cnc(N)c(OC(F)(F)F)c3)nn2C(C)C)C1]

In embodiments, R¹ is

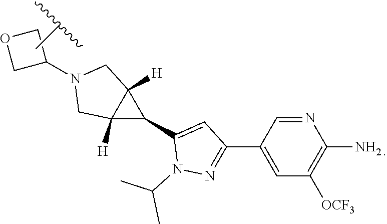

In embodiments, R¹ is

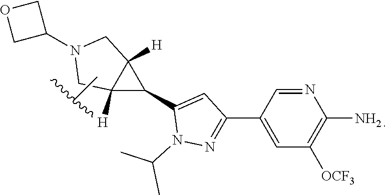

In embodiments, R¹ is

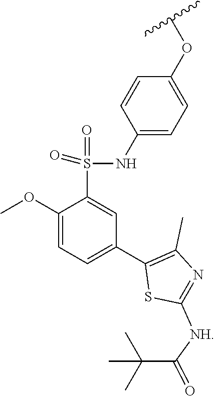 [SMILES: *Oc1ccc(NS(=O)(=O)c2cc(-c3sc(NC(=O)C(C)(C)C)nc3C)ccc2OC)cc1]

In embodiments, R¹ is

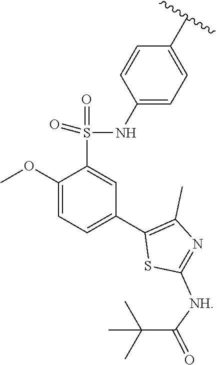 [SMILES: *c1ccc(NS(=O)(=O)c2cc(-c3sc(NC(=O)C(C)(C)C)nc3C)ccc2OC)cc1]

In embodiments, R¹ is

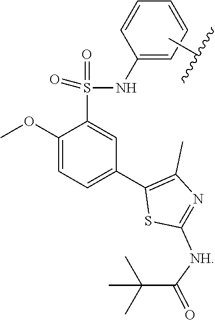

In embodiments, R¹ is

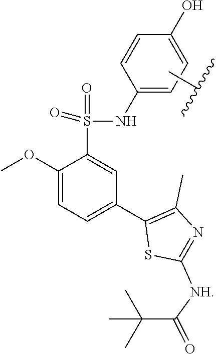

In embodiments, R¹ is

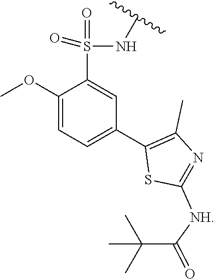 [SMILES: *NS(=O)(=O)c1cc(-c2sc(NC(=O)C(C)(C)C)nc2C)ccc1OC]

In embodiments, R¹ is

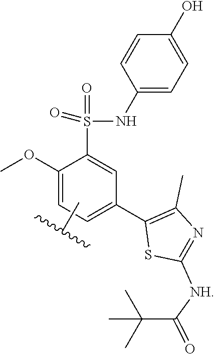

In embodiments, L¹ is substituted (e.g., substituted with at least one substituent group, size-limited substituent group, or lower substituent group) or unsubstituted alkylene (e.g., C₁-C₈, C₁-C₆, C₁-C₄, or C₁-C₂), substituted (e.g., substituted with at least one substituent group, size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkylene (e.g., C₃-C₈, C₃-C₆, C₄-C₆, or C₅-C₆), substituted (e.g., substituted with at least one substituent group, size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted arylene (e.g., C₆-C₁₀ or phenylene), or substituted (e.g., substituted with at least one substituent group, size-limited substituent group, or lower substituent group) or unsubstituted heteroarylene (e.g., 5 to 10 membered, 5 to 9 membered, or 5 to 6 membered).

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is substituted or unsubstituted alkylene, substituted or unsubstituted heteroalkylene, substituted or unsubstituted heterocycloalkylene.

In embodiments, L¹ is substituted or unsubstituted C₁-C₁₀ alkylene, substituted or unsubstituted 2 to 15 membered heteroalkylene, or substituted or unsubstituted 5 to 6 membered heterocycloalkylene.

In embodiments, L¹ is substituted or unsubstituted C₁-C₁₀ alkylene. In embodiments, L¹ is substituted or unsubstituted C₁-C₅ alkylene. In embodiments, L¹ is substituted or unsubstituted C₁-C₃ alkylene. In embodiments, L¹ is substituted or unsubstituted C₁-C₂ alkylene. In embodiments, L¹ is substituted or unsubstituted 2 to 15 membered heteroalkylene. In embodiments, L¹ is substituted or unsubstituted 2 to 10 membered heteroalkylene. In embodiments, L¹ is oxo substituted 2 to 5 membered heteroalkylene. In embodiments, L¹ is substituted or unsubstituted 2 to 5 membered heteroalkylene. In embodiments, L¹ is substituted or unsubstituted 2 to 3 membered heteroalkylene. In embodiments, L¹ is oxo substituted 2 to 3 membered heteroalkylene. In embodiments, L¹ is substituted or unsubstituted 5 to 6 membered heterocycloalkylene. In embodiments, L¹ is substituted or unsubstituted piperazinylene. In embodiments, L¹ is unsubstituted piperazinylene.

In embodiments, L² is —O—, substituted or unsubstituted alkylene, substituted or unsubstituted heteroalkylene, substituted or unsubstituted heterocycloalkylene.

In embodiments, L² is —O—. In embodiments, L² is —NH—. In embodiments, L² is substituted or unsubstituted C₁-C₁₀ alkylene. In embodiments, L² is substituted or unsubstituted C₁-C₅ alkylene. In embodiments, L² is substituted or unsubstituted C₁-C₃ alkylene. In embodiments, L² is substituted or unsubstituted C₁-C₂ alkylene. In embodiments, L² is oxo substituted C₁-C₁₀ alkylene. In embodiments, L² is oxo substituted C₂-C₃ alkylene. In embodiments, L² is substituted or unsubstituted 2 to 15 membered heteroalkylene. In embodiments, L² is substituted or unsubstituted 2 to 10 membered heteroalkylene. In embodiments, L² is substituted or unsubstituted 2 to 5 membered heteroalkylene. In embodiments, L² is substituted or unsubstituted 2 to 3 membered heteroalkylene. In embodiments, L² is oxo substituted 2 to 15 membered heteroalkylene. In embodiments, L² is oxo substituted 2 to 10 membered heteroalkylene. In embodiments, L² is oxo substituted 2 to 5 membered heteroalkylene. In embodiments, L² is oxo substituted 2 to 3 membered heteroalkylene. In embodiments, L² is substituted or unsubstituted 5 to 6 membered heterocycloalkylene. In embodiments, L² is substituted or unsubstituted piperazinylene. In embodiments, L² is unsubstituted piperazinylene.

In embodiments, L³ is substituted or unsubstituted alkylene, substituted or unsubstituted heteroalkylene, substituted or unsubstituted heterocycloalkylene, substituted or unsubstituted arylene, or substituted or unsubstituted heteroarylene.

In embodiments, L³ is substituted or unsubstituted C₁-C₁₀ alkylene. In embodiments, L³ is substituted or unsubstituted C₁-C₅ alkylene. In embodiments, L³ is substituted or unsubstituted C₁-C₃ alkylene. In embodiments, L³ is substituted or unsubstituted C₁-C₂ alkylene. In embodiments, L³ is substituted or unsubstituted C₁-C₂ alkylene. In embodiments, L³ is oxo substituted C₁-C₁₀ alkylene. In embodiments, L³ is oxo substituted C₂-C₃ alkylene. In embodiments, L³ is substituted or unsubstituted 2 to 15 membered heteroalkylene. In embodiments, L³ is substituted or unsubstituted 2 to 10 membered heteroalkylene In embodiments, L³ is substituted or unsubstituted 2 to 5 membered heteroalkylene. In embodiments, L³ is substituted or unsubstituted 2 to 3 membered heteroalkylene. In embodiments, L³ is oxo substituted 2 to 15 membered heteroalkylene. In embodiments, L³ is oxo substituted 2 to 10 membered heteroalkylene. In embodiments, L³ is oxo substituted 2 to 5 membered heteroalkylene. In embodiments, L³ is oxo substituted 2 to 3 membered heteroalkylene. In embodiments, L³ is substituted or unsubstituted 5 to 6 membered heterocycloalkylene. In embodiments, L³ is substituted or unsubstituted piperazinylene. In embodiments, L³ is 5 to 6-membered substituted or unsubstituted arylene. In embodiments, L³ is substituted or unsubstituted phenylene. In embodiments, L³ is 5 to 6-membered substituted or unsubstituted heteroarylene. In embodiments, L³ is substituted or unsubstituted pyridinylene. In embodiments, L³ is substituted or unsubstituted furanylene. In embodiments, L³ is unsubstituted pyridinylene. In embodiments, L³ is unsubstituted furanylene.

In embodiments, L⁴ is —O—. In embodiments, L⁴ is —NH—. In embodiments, L⁴ is substituted or unsubstituted C₁-C₁₀ alkylene. In embodiments, L⁴ is substituted or unsubstituted C₁-C₅ alkylene. In embodiments, L⁴ is substituted or unsubstituted C₁-C₃ alkylene. In embodiments, L⁴ is substituted or unsubstituted C₁-C₂ alkylene. In embodiments, L⁴ is substituted or unsubstituted C₁-C₂ alkylene. In embodiments, L⁴ is oxo substituted C₁-C₁₀ alkylene. In embodiments, L⁴ is oxo substituted C₂-C₃ alkylene. In embodiments, L⁴ is substituted or unsubstituted 2 to 15 membered heteroalkylene. In embodiments, L⁴ is substituted or unsubstituted 2 to 10 membered heteroalkylene. In embodiments, L⁴ is substituted or unsubstituted 2 to 5 membered heteroalkylene. In embodiments, L⁴ is substituted or unsubstituted 2 to 3 membered heteroalkylene. In embodiments, L⁴ is oxo substituted 2 to 15 membered heteroalkylene. In embodiments, L⁴ is oxo substituted 2 to 10 membered heteroalkylene. In embodiments, L⁴ is oxo substituted 2 to 5 membered heteroalkylene. In embodiments, L⁴ is oxo substituted 2 to 3 membered heteroalkylene. In embodiments, L⁴ is substituted or unsubstituted 5 to 6 membered heterocycloalkylene. In embodiments, L⁴ is substituted or unsubstituted piperazinylene. In embodiments, L⁴ is unsubstituted piperazinylene.

In embodiments, L¹ is, —NH—, —NR²³—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹ is L²-L³-L⁴-L⁵-L⁶. In embodiments, L² is connected directly to a monovalent FK506 or a monovalent FK506 analog. In embodiments, L² is connected directly to a monovalent SLF or a monovalent SLF analog. In embodiments, L² is connected directly to a monovalent cyclosporin A or a monovalent cyclosporin A analog. In embodiments, L² is connected directly to a monovalent rapamycin or a monovalent rapamycin analog. In embodiments, L² is connected directly to a monovalent sangliferin A or a monovalent sangliferin A analog. In embodiments, L² is independently a bond, —NH—, —NR²⁶—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is a bond, —NH—, —NR²⁹—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁴ is a bond, —NH—, —NR³²—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁵ is a bond, —NH—, —NR³⁵—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁶ is a bond, —NH—, —NR³⁸—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁴ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁵ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁶ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹ is a divalent linker including one or more amino acids. In embodiments, L¹ is a divalent linker consisting of amino acids. In embodiments, L¹ is a divalent linker including an amino acid analog. In embodiments, L¹ is a divalent linker including an amino acid mimetic. In embodiments, L¹ is a divalent linker consisting of amino acid analogs. In embodiments, L¹ is a divalent linker consisting of amino acid mimetics.

In embodiments, L²

is —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, or —OC(O)—.

In embodiments, L³ is a

bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, or —OC(O)—.

In embodiments, L⁴ is a

bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, or —OC(O)—.

In embodiments, L⁵ is a

bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, or —OC(O)—.

In embodiments, L⁶ is a

bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, or —OC(O)—.

In embodiments, L³ is a bond. In embodiments, L⁴ is a bond. In embodiments, L⁵ is a bond. In embodiments, L⁶ is a bond.

In embodiments, L² is substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L² is substituted or unsubstituted 3 to 8 membered heteroalkylene. In embodiments, L² is —CH₂CH₂OCH₂—. In embodiments, L² is unsubstituted 3 to 8 membered heteroalkylene. In embodiments, L² is unsubstituted 3 to 6 membered heteroalkylene. In embodiments, L² is unsubstituted 3 to 5 membered heteroalkylene. In embodiments, L² is a divalent linker including one or more amino acids. In embodiments, L² is a divalent linker consisting of amino acids. In embodiments, L² is a divalent linker including an amino acid analog. In embodiments, L² is a divalent linker including an amino acid mimetic. In embodiments, L² is a divalent linker consisting of amino acid analogs. In embodiments, L² is a divalent linker consisting of amino acid mimetics. In embodiments, L² is a bioconjugate linker.

In embodiments, L³ is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L³ is a substituted or unsubstituted 5 to 10 membered heteroarylene. In embodiments, L³ is a bond. In embodiments, L³ is a substituted or unsubstituted 5 to 6 membered heteroarylene. In embodiments, L³ is a unsubstituted 5 to 6 membered heteroarylene. In embodiments, L³ is unsubstituted divalent triazole. In embodiments, L³ is unsubstituted divalent 1H-1,2,3-triazole. In embodiments, L³ is unsubstituted divalent 2H-1,2,3-triazole. In embodiments, L³ is a divalent linker including one or more amino acids. In embodiments, L³ is a divalent linker consisting of amino acids. In embodiments, L³ is a divalent linker including an amino acid analog. In embodiments, L³ is a divalent linker including an amino acid mimetic. In embodiments, L³ is a divalent linker consisting of amino acid analogs. In embodiments, L³ is a divalent linker consisting of amino acid mimetics. In embodiments, L³ is a bioconjugate linker.

In embodiments, L⁴ is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L⁴ is a substituted or unsubstituted 2 to 12 membered heteroalkylene. In embodiments, L⁴ is an unsubstituted 2 to 12 membered heteroalkylene. In embodiments, L⁴ is a bond. In embodiments, L⁴ is a divalent linker including one or more amino acids. In embodiments, L⁴ is a divalent linker consisting of amino acids. In embodiments, L⁴ is a divalent linker including an amino acid analog. In embodiments, L⁴ is a divalent linker including an amino acid mimetic. In embodiments, L⁴ is a divalent linker consisting of amino acid analogs. In embodiments, L⁴ is a divalent linker consisting of amino acid mimetics. In embodiments, L⁴ is a bioconjugate linker.

In embodiments, L⁵ is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L⁵ is a substituted or unsubstituted 2 to 12 membered heteroalkylene. In embodiments, L⁵ is an unsubstituted 2 to 12 membered heteroalkylene. In embodiments, L⁵ is a bond. In embodiments, L⁵ is a divalent linker including one or more amino acids. In embodiments, L⁵ is a divalent linker consisting of amino acids. In embodiments, L⁵ is a divalent linker including an amino acid analog. In embodiments, L⁵ is a divalent linker including an amino acid mimetic. In embodiments, L⁵ is a divalent linker consisting of amino acid analogs. In embodiments, L⁵ is a divalent linker consisting of amino acid mimetics. In embodiments, L⁵ is a bioconjugate linker.

In embodiments, L⁶ is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L⁶ is a substituted or unsubstituted 2 to 12 membered heteroalkylene. In embodiments, L⁶ is an unsubstituted 2 to 12 membered heteroalkylene. In embodiments, L⁶ is a bond. In embodiments, L⁶ is a divalent linker including one or more amino acids. In embodiments, L⁶ is a divalent linker consisting of amino acids. In embodiments, L⁶ is a divalent linker including an amino acid analog. In embodiments, L⁶ is a divalent linker including an amino acid mimetic. In embodiments, L⁶ is a divalent linker consisting of amino acid analogs. In embodiments, L⁶ is a divalent linker consisting of amino acid mimetics. In embodiments, L⁶ is a bioconjugate linker.

In embodiments, L⁵ is a divalent oligomer of ethylene oxide. In embodiments, L⁵ is a divalent polyethylene glycol. In embodiments, L⁵ is a divalent oligomer of ethylene oxide having 2 to 30 linear atoms (carbon and oxygen) between the two termini connecting to the remainder of the compound. In embodiments, L⁵ is a —(CH₂)₄C(O)NH—. In embodiments, L⁵ is a 2 to 8 membered substituted heteroalkylene. In embodiments, L⁵ is a 3 to 6 membered substituted heteroalkylene. In embodiments, L⁵ is a 5 to 6 membered substituted heteroalkylene. In embodiments, L⁵ is a 5 to 7 membered oxo substituted heteroalkylene. In embodiments, L⁵ is an unsubstituted C₁-C₆ alkylene.

In embodiments, L⁴ is a divalent oligomer of ethylene oxide. In embodiments, L⁴ is a divalent polyethylene glycol. In embodiments, L⁴ is a divalent oligomer of ethylene oxide having 2 to 30 linear atoms (carbon and oxygen) between the two termini connecting to the remainder of the compound. In embodiments, L⁴ is —(CH₂CH₂O)_{b}CH₂CH₂— and b is an integer from 1 to 16. In embodiments, L⁴ is —(CH₂CH₂O)_{b}CH₂— and b is an integer from 1 to 16. In embodiments, L⁴ is —(CH₂CH₂O)_{b}— and b is an integer from 1 to 16. In embodiments, b is an integer from 2 to 15. In embodiments, b is an integer from 3 to 14. In embodiments, b is an integer from 4 to 12. In embodiments, b is an integer from 5 to 10. In embodiments, b is an integer from 5 to 8. In embodiments, b is an integer from 6 to 7.

In embodiments, L⁴-L⁵ is a 2 to 36 membered substituted heteroalkylene. In embodiments, L⁴-L⁵ is a 2 to 34 membered substituted heteroalkylene. In embodiments, L⁴-L⁵ is a 2 to 32 membered substituted heteroalkylene. In embodiments, L⁴-L⁵ is a 2 to 30 membered substituted heteroalkylene. In embodiments, L⁴-L⁵ is a 2 to 28 membered substituted heteroalkylene. In embodiments, L⁴-L⁵ is a 2 to 24 membered substituted heteroalkylene. In embodiments, L⁴-L⁵ is a 2 to 30 membered substituted heteroalkylene. In embodiments, L⁴-L⁵ is a 2 to 22 membered substituted heteroalkylene. In embodiments, L⁴-L⁵ is a 2 to 20 membered substituted heteroalkylene. In embodiments, L⁴-L⁵ is a 2 to 18 membered substituted heteroalkylene. In embodiments, L⁴-L⁵ is a 2 to 16 membered substituted heteroalkylene. In embodiments, L⁴-L⁵ is a 2 to 14 membered substituted heteroalkylene. In embodiments, L⁴-L⁵ is a 2 to 12 membered substituted heteroalkylene. In embodiments, L⁴-L⁵ is a 4 to 36 membered substituted heteroalkylene. In embodiments, L⁴-L⁵ is a 6 to 36 membered substituted heteroalkylene. In embodiments, L⁴-L⁵ is a 8 to 36 membered substituted heteroalkylene. In embodiments, L⁴-L⁵ is a 10 to 36 membered substituted heteroalkylene. In embodiments, L⁴-L⁵ is a 12 to 36 membered substituted heteroalkylene. In embodiments, L⁴-L⁵ is a 14 to 36 membered substituted heteroalkylene. In embodiments, L⁴-L⁵ is a 16 to 36 membered substituted heteroalkylene. In embodiments, L⁴-L⁵ is a 18 to 36 membered substituted heteroalkylene. In embodiments, L⁴-L⁵ is a 20 to 36 membered substituted heteroalkylene. In embodiments, L⁴-L⁵ is a 22 to 36 membered substituted heteroalkylene. In embodiments, L⁴-L⁵ is a 24 to 36 membered substituted heteroalkylene. In embodiments, L⁴-L⁵ is a 4 to 32 membered substituted heteroalkylene. In embodiments, L⁴-L⁵ is a 4 to 28 membered substituted heteroalkylene. In embodiments, L⁴-L⁵ is a 8 to 26 membered substituted heteroalkylene. In embodiments, L⁴-L⁵ is a 12 to 26 membered substituted heteroalkylene. In embodiments, L⁴-L⁵ is a 16 to 26 membered substituted heteroalkylene. In embodiments, L⁴-L⁵ is a 20 to 26 membered substituted heteroalkylene. In embodiments, L⁴-L⁵ is a 22 to 26 membered substituted heteroalkylene.

In embodiments, the linker is formed by a conjugation or bioconjugation reaction combining a first reactant moiety covalently bonded to the immunophilin binding moiety and a second reactant moiety covalently bonded to the R¹ moiety. In such embodiments, the compound formed by such conjugation or bioconjugation reaction (including compounds as described herein) may be referred to as a conjugate or bioconjugate or bioconjugate linker.

In embodiments, L¹ is independently R²³-substituted or unsubstituted alkylene, R²³-substituted or unsubstituted heteroalkylene, R²³-substituted or unsubstituted cycloalkylene, R²³-substituted or unsubstituted heterocycloalkylene, R²³-substituted or unsubstituted arylene, or R²³-substituted or unsubstituted heteroarylene.

In embodiments, L¹ is a

bond, —NH—, —NR²³—, —S—, —O—, —C(O)—, —NHC(O)—, —C(O)NH—, —NHC(O)NH—, —NHC(NH)NH—, —C(S)—, R²³-substituted or unsubstituted C₁-C₂₀ alkylene, R²³-substituted or unsubstituted 2 to 20 membered heteroalkylene, R²³-substituted or unsubstituted C₃-C₈ cycloalkylene, R²³-substituted or unsubstituted 3 to 8 membered heterocycloalkylene, R²³-substituted or unsubstituted C₆-C₁₀ arylene, or R²³-substituted or unsubstituted 5 to 10 membered heteroarylene. In embodiments, L¹ is a bond. In embodiments, L¹ is —NH—. In embodiments, L¹ is —NR²³—. In embodiments, L¹ is —S—. In embodiments, L¹ is —O—. In embodiments, L¹ is —C(O)—. In embodiments, L¹ is —NHC(O)—. In embodiments, L¹ is —C(O)NH—. In embodiments, L¹ is —NHC(O)NH—. In embodiments, L¹ is —NHC(NH)NH—. In embodiments, L¹ is —C(S)—. In embodiments, L¹ is R²³-substituted or unsubstituted C₁-C₂₀ alkylene. In embodiments, L¹ is R²³-substituted or unsubstituted 2 to 20 membered heteroalkylene. In embodiments, L¹ is R²³-substituted or unsubstituted C₃-C₈ cycloalkylene. In embodiments, L¹ is R²³-substituted or unsubstituted 3 to 8 membered heterocycloalkylene. In embodiments, L¹ is R²³-substituted or unsubstituted C₆-C₁₀ arylene. In embodiments, L¹ is R²³-substituted or unsubstituted 5 to 10 membered heteroarylene. In embodiments, L¹ is R²³-substituted C₁-C₂₀ alkylene. In embodiments, L¹ is R²³-substituted 2 to 20 membered heteroalkylene. In embodiments, L¹ is R²³-substituted C₃-C₈ cycloalkylene. In embodiments, L¹ is R²³-substituted 3 to 8 membered heterocycloalkylene. In embodiments, L¹ is R²³-substituted C₆-C₁₀ arylene. In embodiments, L¹ is R²³-substituted 5 to 10 membered heteroarylene. In embodiments, L¹ is unsubstituted C₁-C₂₀ alkylene. In embodiments, L¹ is unsubstituted 2 to 20 membered heteroalkylene. In embodiments, L¹ is unsubstituted C₃-C₈ cycloalkylene. In embodiments, L¹ is unsubstituted 3 to 8 membered heterocycloalkylene. In embodiments, L¹ is unsubstituted C₆-C₁₀ arylene. In embodiments, L¹ is unsubstituted 5 to 10 membered heteroarylene. In embodiments, L¹ is R²³-substituted C₁-C₁₅ alkylene. In embodiments, L¹ is R²³-substituted 2 to 15 membered heteroalkylene. In embodiments, L¹ is R²³-substituted C₃-C₆ cycloalkylene. In embodiments, L¹ is R²³-substituted 3 to 6 membered heterocycloalkylene. In embodiments, L¹ is R²³-substituted phenylene. In embodiments, L¹ is R²³-substituted 5 to 6 membered heteroarylene. In embodiments, L¹ is unsubstituted C₁-C₁₅ alkylene. In embodiments, L¹ is unsubstituted 2 to 15 membered heteroalkylene. In embodiments, L¹ is unsubstituted C₃-C₆ cycloalkylene. In embodiments, L¹ is unsubstituted 3 to 6 membered heterocycloalkylene. In embodiments, L¹ is unsubstituted phenylene. In embodiments, L¹ is unsubstituted 5 to 6 membered heteroarylene. In embodiments, L¹ is R²³-substituted C₁-C₁₀ alkylene. In embodiments, L¹ is R²³-substituted 2 to 10 membered heteroalkylene. In embodiments, L¹ is R²³-substituted C₄-C₆ cycloalkylene. In embodiments, L¹ is R²³-substituted 4 to 6 membered heterocycloalkylene. In embodiments, L¹ is R²³-substituted phenylene. In embodiments, L¹ is R²³-substituted 5 membered heteroarylene. In embodiments, L¹ is R²³-substituted C₁-C₈ alkylene. In embodiments, L¹ is R²³-substituted 2 to 8 membered heteroalkylene. In embodiments, L¹ is R²³-substituted C₅-C₆ cycloalkylene. In embodiments, L¹ is R²³-substituted 5 to 6 membered heterocycloalkylene. In embodiments, L¹ is R²³-substituted 6 membered heteroarylene. In embodiments, L¹ is R²³-substituted C₁-C₆ alkylene. In embodiments, L¹ is R²³-substituted 2 to 6 membered heteroalkylene. In embodiments, L¹ is R²³-substituted C₆-C₂₀ alkylene. In embodiments, L¹ is R²³-substituted 6 to 20 membered heteroalkylene. In embodiments, L¹ is unsubstituted C₁-C₁₀ alkylene. In embodiments, L¹ is unsubstituted 2 to 10 membered heteroalkylene. In embodiments, L¹ is unsubstituted C₄-C₆ cycloalkylene. In embodiments, L¹ is unsubstituted 4 to 6 membered heterocycloalkylene. In embodiments, L¹ is unsubstituted phenylene. In embodiments, L¹ is unsubstituted 5 membered heteroarylene. In embodiments, L¹ is unsubstituted C₁-C₈ alkylene. In embodiments, L¹ is unsubstituted 2 to 8 membered heteroalkylene. In embodiments, L¹ is unsubstituted C₅-C₆ cycloalkylene. In embodiments, L¹ is unsubstituted 5 to 6 membered heterocycloalkylene. In embodiments, L¹ is unsubstituted 6 membered heteroarylene. In embodiments, L¹ is unsubstituted C₁-C₆ alkylene. In embodiments, L¹ is unsubstituted 2 to 6 membered heteroalkylene. In embodiments, L¹ is unsubstituted C₆-C₂₀ alkylene. In embodiments, L¹ is unsubstituted 6 to 20 membered heteroalkylene.

R²³ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R²⁴-substituted or unsubstituted alkyl, R²⁴-substituted or unsubstituted heteroalkyl, R²⁴-substituted or unsubstituted cycloalkyl, R²⁴-substituted or unsubstituted heterocycloalkyl, R²⁴-substituted or unsubstituted aryl, or R²⁴-substituted or unsubstituted heteroaryl.

In embodiments, R²³ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R²⁴-substituted or unsubstituted alkyl (e.g., C₁-C₈, C₁-C₆, C₁-C₄, or C₁-C₂), R²⁴-substituted or unsubstituted heteroalkyl (e.g., 2 to 8 membered, 2 to 6 membered, 4 to 6 membered, 2 to 3 membered, or 4 to 5 membered), R²⁴-substituted or unsubstituted cycloalkyl (e.g., C₃-C₈, C₃-C₆, C₄-C₆, or C₅-C₆), R²⁴-substituted or unsubstituted heterocycloalkyl (e.g., 3 to 8 membered, 3 to 6 membered, 4 to 6 membered, 4 to 5 membered, or 5 to 6 membered), R²⁴-substituted or unsubstituted aryl (e.g., C₆-C₁₀ or phenyl), or R²⁴-substituted or unsubstituted heteroaryl (e.g., 5 to 10 membered, 5 to 9 membered, or 5 to 6 membered).

In embodiments, R²³ is independently —NH₂. In embodiments, R²³ is independently —OH. In embodiments, R²³ is independently halogen. In embodiments, R²³ is independently —CN. In embodiments, R²³ is independently oxo. In embodiments, R²³ is independently —CF₃. In embodiments, R²³ is independently —COOH. In embodiments, R²³ is independently —CONH₂. In embodiments, R²³ is independently —NO₂. In embodiments, R²³ is independently —SH. In embodiments, R²³ is independently —SO₃H. In embodiments, R²³ is independently —SO₄H. In embodiments, R²³ is independently —SO₂NH₂. In embodiments, R²³ is independently —NHNH₂. In embodiments, R²³ is independently —ONH₂. In embodiments, R²³ is independently —NHC═(O)NHNH₂. In embodiments, R²³ is independently —NHC═(O)NH₂. In embodiments, R²³ is independently —NHSO₂H. In embodiments, R²³ is independently —NHC═(O)H. In embodiments, R²³ is independently —NHC(O)—OH. In embodiments, R²³ is independently —NHOH. In embodiments, R²³ is independently —OCF₃. In embodiments, R²³ is independently —OCHF₂. In embodiments, R²³ is independently —CCl₃. In embodiments, R²³ is independently —CBr₃. In embodiments, R²³ is independently —CI₃. In embodiments, R²³ is independently —F. In embodiments, R²³ is independently —Cl. In embodiments, R²³ is independently —Br. In embodiments, R²³ is independently —I. In embodiments, R²³ is independently R²⁴-substituted C₁-C₄ alkyl. In embodiments, R²³ is independently R²⁴-substituted 2 to 4 membered heteroalkyl. In embodiments, R²³ is independently R²⁴-substituted C₃-C₆ cycloalkyl. In embodiments, R²³ is independently R²⁴-substituted 3 to 6 membered heterocycloalkyl. In embodiments, R²³ is independently R²⁴-substituted phenyl. In embodiments, R²³ is independently R²⁴-substituted 5 to 6 membered heteroaryl. In embodiments, R²³ is independently unsubstituted C₁-C₄ alkyl. In embodiments, R²³ is independently unsubstituted 2 to 4 membered heteroalkyl. In embodiments, R²³ is independently unsubstituted C₃-C₆ cycloalkyl. In embodiments, R²³ is independently unsubstituted 3 to 6 membered heterocycloalkyl. In embodiments, R²³ is independently unsubstituted phenyl. In embodiments, R²³ is independently unsubstituted 5 to 6 membered heteroaryl.

R²⁴ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, or R²⁵-substituted or unsubstituted heteroaryl.

In embodiments, R²⁴ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R²⁵-substituted or unsubstituted alkyl (e.g., C₁-C₈, C₁-C₆, C₁-C₄, or C₁-C₂), R²⁴-substituted or unsubstituted heteroalkyl (e.g., 2 to 8 membered, 2 to 6 membered, 4 to 6 membered, 2 to 3 membered, or 4 to 5 membered), R²⁵-substituted or unsubstituted cycloalkyl (e.g., C₃-C₈, C₃-C₆, C₄-C₆, or C₅-C₆), R²⁵-substituted or unsubstituted heterocycloalkyl (e.g., 3 to 8 membered, 3 to 6 membered, 4 to 6 membered, 4 to 5 membered, or 5 to 6 membered), R²⁵-substituted or unsubstituted aryl (e.g., C₆-C₁₀ or phenyl), or R²⁵-substituted or unsubstituted heteroaryl (e.g., 5 to 10 membered, 5 to 9 membered, or 5 to 6 membered).

In embodiments, R²⁴ is independently —NH₂. In embodiments, R²⁴ is independently —OH. In embodiments, R²⁴ is independently halogen. In embodiments, R²⁴ is independently —CN. In embodiments, R²⁴ is independently oxo. In embodiments, R²⁴ is independently —CF₃. In embodiments, R²⁴ is independently —COOH. In embodiments, R²⁴ is independently —CONH₂. In embodiments, R²⁴ is independently —NO₂. In embodiments, R²⁴ is independently —SH. In embodiments, R²⁴ is independently —SO₃H. In embodiments, R²⁴ is independently —SO₄H. In embodiments, R²⁴ is independently —SO₂NH₂. In embodiments, R²⁴ is independently —NHNH₂. In embodiments, R²⁴ is independently —ONH₂. In embodiments, R²⁴ is independently —NHC═(O)NHNH₂. In embodiments, R²⁴ is independently —NHC═(O) NH₂. In embodiments, R²⁴ is independently —NHSO₂H. In embodiments, R²⁴ is independently —NHC═(O)H. In embodiments, R²⁴ is independently —NHC(O)—OH. In embodiments, R²⁴ is independently —NHOH. In embodiments, R²⁴ is independently —OCF₃. In embodiments, R²⁴ is independently —OCHF₂. In embodiments, R²⁴ is independently —CCl₃. In embodiments, R²⁴ is independently —CBr₃. In embodiments, R²⁴ is independently —CI₃. In embodiments, R²⁴ is independently —F. In embodiments, R²⁴ is independently —Cl. In embodiments, R²⁴ is independently —Br. In embodiments, R²⁴ is independently —I. In embodiments, R²⁴ is independently R²⁵-substituted C₁-C₄ alkyl. In embodiments, R²⁴ is independently R²⁵-substituted 2 to 4 membered heteroalkyl. In embodiments, R²⁴ is independently R²⁵-substituted C₃-C₆ cycloalkyl. In embodiments, R²⁴ is independently R⁵-substituted 3 to 6 membered heterocycloalkyl. In embodiments, R²⁴ is independently R²⁵-substituted phenyl. In embodiments, R²⁴ is independently R²⁵-substituted 5 to 6 membered heteroaryl. In embodiments, R²⁴ is independently unsubstituted C₁-C₄ alkyl. In embodiments, R²⁴ is independently unsubstituted 2 to 4 membered heteroalkyl. In embodiments, R²⁴ is independently unsubstituted C₃-C₆ cycloalkyl. In embodiments, R²⁴ is independently unsubstituted 3 to 6 membered heterocycloalkyl. In embodiments, R²⁴ is independently unsubstituted phenyl. In embodiments, R²⁴ is independently unsubstituted 5 to 6 membered heteroaryl.

In embodiments, L² is independently a bond, R²⁶-substituted or unsubstituted alkylene, R²⁶-substituted or unsubstituted heteroalkylene, R²⁶-substituted or unsubstituted cycloalkylene, R²⁶-substituted or unsubstituted heterocycloalkylene, R²⁶-substituted or unsubstituted arylene, or R²⁶-substituted or unsubstituted heteroarylene.

In embodiments, L² is independently bond, R²⁶-substituted or unsubstituted alkylene, R²⁶-substituted or unsubstituted heteroalkylene, R²⁶-substituted or unsubstituted cycloalkylene, R²⁶-substituted or unsubstituted heterocycloalkylene, R²⁶-substituted or unsubstituted arylene, or R²⁶-substituted or unsubstituted heteroarylene.

In embodiments, L² is independently a

bond, —NH—, —NR²⁶—, —S—, —O—, —C(O)—, —NHC(O)—, —C(O)NH—, —NHC(O)NH—, —NHC(NH)NH—, —C(S)—, R²⁶-substituted or unsubstituted C₁-C₂₀ alkylene, R²⁶-substituted or unsubstituted 2 to 20 membered heteroalkylene, R²⁶-substituted or unsubstituted C₃-C₈ cycloalkylene, R²⁶-substituted or unsubstituted 3 to 8 membered heterocycloalkylene, R²⁶-substituted or unsubstituted C₆-C₁₀ arylene, or R²⁶-substituted or unsubstituted 5 to 10 membered heteroarylene. In embodiments, L² is —NH—. In embodiments, L² is —NR²⁶—. In embodiments, L² is —S—. In embodiments, L² is —O—. In embodiments, L² is —C(O)—. In embodiments, L² is —NHC(O)—. In embodiments, L² is —C(O)NH—. In embodiments, L² is —NHC(O)NH—. In embodiments, L² is —NHC(NH)NH—. In embodiments, L² is —C(S)—. In embodiments, L² is R²⁶-substituted or unsubstituted C₁-C₂₀ alkylene. In embodiments, L² is R²⁶-substituted or unsubstituted 2 to 20 membered heteroalkylene. In embodiments, L² is R²⁶-substituted or unsubstituted C₃-C₈ cycloalkylene. In embodiments, L² is R²⁶-substituted or unsubstituted 3 to 8 membered heterocycloalkylene. In embodiments, L² is R²⁶-substituted or unsubstituted C₆-C₁₀ arylene. In embodiments, L² is R²⁶-substituted or unsubstituted 5 to 10 membered heteroarylene. In embodiments, L² is R²⁶-substituted C₁-C₂₀ alkylene. In embodiments, L² is R²⁶-substituted 2 to 20 membered heteroalkylene. In embodiments, L² is R²⁶-substituted C₃-C₅ cycloalkylene. In embodiments, L² is R²⁶-substituted 3 to 8 membered heterocycloalkylene. In embodiments, L² is R²⁶-substituted C₆-C₁₀ arylene. In embodiments, L² is R²⁶-substituted 5 to 10 membered heteroarylene. In embodiments, L² is unsubstituted C₁-C₂₀ alkylene. In embodiments, L² is unsubstituted 2 to 20 membered heteroalkylene. In embodiments, L² is unsubstituted C₃-C₈ cycloalkylene. In embodiments, L² is unsubstituted 3 to 8 membered heterocycloalkylene. In embodiments, L² is unsubstituted C₆-C₁₀ arylene. In embodiments, L² is unsubstituted 5 to 10 membered heteroarylene. In embodiments, L² is R²⁶-substituted C₁-C₁₅ alkylene. In embodiments, L² is R²⁶-substituted 2 to 15 membered heteroalkylene. In embodiments, L² is R²⁶-substituted C₃-C₆ cycloalkylene. In embodiments, L² is R²⁶-substituted 3 to 6 membered heterocycloalkylene. In embodiments, L² is R²⁶-substituted phenylene. In embodiments, L² is R²⁶-substituted 5 to 6 membered heteroarylene. In embodiments, L² is unsubstituted C₁-C₁₅ alkylene. In embodiments, L² is unsubstituted 2 to 15 membered heteroalkylene. In embodiments, L² is unsubstituted C₃-C₆ cycloalkylene. In embodiments, L² is unsubstituted 3 to 6 membered heterocycloalkylene. In embodiments, L² is unsubstituted phenylene. In embodiments, L² is unsubstituted 5 to 6 membered heteroarylene. In embodiments, L² is R²⁶-substituted C₁-C₁₀ alkylene. In embodiments, L² is R²⁶-substituted 2 to 10 membered heteroalkylene. In embodiments, L² is R²⁶-substituted C₄-C₆ cycloalkylene. In embodiments, L² is R²⁶-substituted 4 to 6 membered heterocycloalkylene. In embodiments, L² is R²⁶-substituted phenylene. In embodiments, L² is R²⁶-substituted 5 membered heteroarylene. In embodiments, L² is R²⁶-substituted C₁-C₈ alkylene. In embodiments, L² is R²⁶-substituted 2 to 8 membered heteroalkylene. In embodiments, L² is R²⁶-substituted C₅-C₆ cycloalkylene. In embodiments, L² is R²⁶-substituted 5 to 6 membered heterocycloalkylene. In embodiments, L² is R²⁶-substituted 6 membered heteroarylene. In embodiments, L² is R²⁶-substituted C₁-C₆ alkylene. In embodiments, L² is R²⁶-substituted 2 to 6 membered heteroalkylene. In embodiments, L² is R²⁶-substituted C₆-C₂₀ alkylene. In embodiments, L² is R²⁶-substituted 6 to 20 membered heteroalkylene. In embodiments, L² is unsubstituted C₁-C₁₀ alkylene. In embodiments, L² is unsubstituted 2 to 10 membered heteroalkylene. In embodiments, L² is unsubstituted C₄-C₆ cycloalkylene. In embodiments, L² is unsubstituted 4 to 6 membered heterocycloalkylene. In embodiments, L² is unsubstituted phenylene. In embodiments, L² is unsubstituted 5 membered heteroarylene. In embodiments, L² is unsubstituted C₁-C₈ alkylene. In embodiments, L² is unsubstituted 2 to 8 membered heteroalkylene. In embodiments, L² is unsubstituted C₅-C₆ cycloalkylene. In embodiments, L² is unsubstituted 5 to 6 membered heterocycloalkylene. In embodiments, L² is unsubstituted 6 membered heteroarylene. In embodiments, L² is unsubstituted C₁-C₆ alkylene. In embodiments, L² is unsubstituted 2 to 6 membered heteroalkylene. In embodiments, L² is unsubstituted C₆-C₂₀ alkylene. In embodiments, L² is unsubstituted 6 to 20 membered heteroalkylene.

R²⁶ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R²⁷-substituted or unsubstituted alkyl, R²⁷-substituted or unsubstituted heteroalkyl, R²⁷-substituted or unsubstituted cycloalkyl, R²⁷-substituted or unsubstituted heterocycloalkyl, R²⁷-substituted or unsubstituted aryl, or R²⁷-substituted or unsubstituted heteroaryl.

In embodiments, R²⁶ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R²⁷-substituted or unsubstituted alkyl (e.g., C₁-C₈, C₁-C₆, C₁-C₄, or C₁-C₂), R²⁷-substituted or unsubstituted heteroalkyl (e.g., 2 to 8 membered, 2 to 6 membered, 4 to 6 membered, 2 to 3 membered, or 4 to 5 membered), R²⁷-substituted or unsubstituted cycloalkyl (e.g., C₃-C₈, C₃-C₆, C₄-C₆, or C₅-C₆), R²⁷-substituted or unsubstituted heterocycloalkyl (e.g., 3 to 8 membered, 3 to 6 membered, 4 to 6 membered, 4 to 5 membered, or 5 to 6 membered), R²⁷-substituted or unsubstituted aryl (e.g., C₆-C₁₀ or phenyl), or R²⁷-substituted or unsubstituted heteroaryl (e.g., 5 to 10 membered, 5 to 9 membered, or 5 to 6 membered).

In embodiments, R²⁶ is independently —NH₂. In embodiments, R²⁶ is independently —OH. In embodiments, R²⁶ is independently halogen. In embodiments, R²⁶ is independently —CN. In embodiments, R²⁶ is independently oxo. In embodiments, R²⁶ is independently —CF₃. In embodiments, R²⁶ is independently —COOH. In embodiments, R²⁶ is independently —CONH₂. In embodiments, R²⁶ is independently —NO₂. In embodiments, R²⁶ is independently —SH. In embodiments, R²⁶ is independently —SO₃H. In embodiments, R²⁶ is independently —SO₄H. In embodiments, R²⁶ is independently —SO₂NH₂. In embodiments, R²⁶ is independently —NHNH₂. In embodiments, R²⁶ is independently —ONH₂. In embodiments, R²⁶ is independently —NHC═(O)NHNH₂. In embodiments, R²⁶ is independently —NHC═(O) NH₂. In embodiments, R²⁶ is independently —NHSO₂H. In embodiments, R²⁶ is independently —NHC═(O)H. In embodiments, R²⁶ is independently —NHC(O)—OH. In embodiments, R²⁶ is independently —NHOH. In embodiments, R²⁶ is independently —OCF₃. In embodiments, R²⁶ is independently —OCHF₂. In embodiments, R²⁶ is independently —CCl₃. In embodiments, R²⁶ is independently —CBr₃. In embodiments, R²⁶ is independently —CI₃. In embodiments, R²⁶ is independently —F. In embodiments, R²⁶ is independently —Cl. In embodiments, R²⁶ is independently —Br. In embodiments, R²⁶ is independently —I. In embodiments, R²⁶ is independently R²⁷-substituted C₁-C₄ alkyl. In embodiments, R²⁶ is independently R²⁷-substituted 2 to 4 membered heteroalkyl. In embodiments, R²⁶ is independently R²⁷-substituted C₃-C₆ cycloalkyl. In embodiments, R²⁶ is independently R²⁷-substituted 3 to 6 membered heterocycloalkyl. In embodiments, R²⁶ is independently R²⁷-substituted phenyl. In embodiments, R²⁶ is independently R²⁷-substituted 5 to 6 membered heteroaryl. In embodiments, R²⁶ is independently unsubstituted C₁-C₄ alkyl. In embodiments, R²⁶ is independently unsubstituted 2 to 4 membered heteroalkyl. In embodiments, R²⁶ is independently unsubstituted C₃-C₆ cycloalkyl. In embodiments, R²⁶ is independently unsubstituted 3 to 6 membered heterocycloalkyl. In embodiments, R²⁶ is independently unsubstituted phenyl. In embodiments, R²⁶ is independently unsubstituted 5 to 6 membered heteroaryl.

R²⁷ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R²⁸-substituted or unsubstituted alkyl, R²⁸-substituted or unsubstituted heteroalkyl, R²⁸-substituted or unsubstituted cycloalkyl, R²⁸-substituted or unsubstituted heterocycloalkyl, R²⁸-substituted or unsubstituted aryl, or R²⁸-substituted or unsubstituted heteroaryl.

In embodiments, R²⁷ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R²⁸-substituted or unsubstituted alkyl (e.g., C₁-C₈, C₁-C₆, C₁-C₄, or C₁-C₂), R²⁸-substituted or unsubstituted heteroalkyl (e.g., 2 to 8 membered, 2 to 6 membered, 4 to 6 membered, 2 to 3 membered, or 4 to 5 membered), R²⁸-substituted or unsubstituted cycloalkyl (e.g., C₃-C₈, C₃-C₆, C₄-C₆, or C₅-C₆), R²⁸-substituted or unsubstituted heterocycloalkyl (e.g., 3 to 8 membered, 3 to 6 membered, 4 to 6 membered, 4 to 5 membered, or 5 to 6 membered), R²⁸-substituted or unsubstituted aryl (e.g., C₆-C₁₀ or phenyl), or R²⁸-substituted or unsubstituted heteroaryl (e.g., 5 to 10 membered, 5 to 9 membered, or 5 to 6 membered).

In embodiments, R²⁷ is independently —NH₂. In embodiments, R²⁷ is independently —OH. In embodiments, R²⁷ is independently halogen. In embodiments, R²⁷ is independently —CN. In embodiments, R²⁷ is independently oxo. In embodiments, R²⁷ is independently —CF₃. In embodiments, R²⁷ is independently —COOH. In embodiments, R²⁷ is independently —CONH₂. In embodiments, R²⁷ is independently —NO₂. In embodiments, R²⁷ is independently —SH. In embodiments, R²⁷ is independently —SO₃H. In embodiments, R²⁷ is independently —SO₄H. In embodiments, R²⁷ is independently —SO₂NH₂. In embodiments, R²⁷ is independently —NHNH₂. In embodiments, R²⁷ is independently —ONH₂. In embodiments, R²⁷ is independently —NHC═(O)NHNH₂. In embodiments, R²⁷ is independently —NHC═(O) NH₂. In embodiments, R²⁷ is independently —NHSO₂H. In embodiments, R²⁷ is independently —NHC═(O)H. In embodiments, R²⁷ is independently —NHC(O)—OH. In embodiments, R²⁷ is independently —NHOH. In embodiments, R²⁷ is independently —OCF₃. In embodiments, R²⁷ is independently —OCHF₂. In embodiments, R²⁷ is independently —CCl₃. In embodiments, R²⁷ is independently —CBr₃. In embodiments, R²⁷ is independently —CI₃. In embodiments, R²⁷ is independently —F. In embodiments, R²⁷ is independently —Cl. In embodiments, R²⁷ is independently —Br. In embodiments, R²⁷ is independently —I. In embodiments, R²⁷ is independently R²⁸-substituted C₁-C₄ alkyl. In embodiments, R²⁷ is independently R²⁸-substituted 2 to 4 membered heteroalkyl. In embodiments, R²⁷ is independently R²⁸-substituted C₃-C₆ cycloalkyl. In embodiments, R²⁷ is independently R²⁸-substituted 3 to 6 membered heterocycloalkyl. In embodiments, R²⁷ is independently R²⁸-substituted phenyl. In embodiments, R²⁷ is independently R²⁸-substituted 5 to 6 membered heteroaryl. In embodiments, R²⁷ is independently unsubstituted C₁-C₄ alkyl. In embodiments, R²⁷ is independently unsubstituted 2 to 4 membered heteroalkyl. In embodiments, R²⁷ is independently unsubstituted C₃-C₆ cycloalkyl. In embodiments, R²⁷ is independently unsubstituted 3 to 6 membered heterocycloalkyl. In embodiments, R²⁷ is independently unsubstituted phenyl. In embodiments, R²⁷ is independently unsubstituted 5 to 6 membered heteroaryl.

In embodiments, L³ is independently a bond, R²⁹-substituted or unsubstituted alkylene, R²⁹-substituted or unsubstituted heteroalkylene, R²⁹-substituted or unsubstituted cycloalkylene, R²⁹-substituted or unsubstituted heterocycloalkylene, R²⁹-substituted or unsubstituted arylene, or R²⁹-substituted or unsubstituted heteroarylene.

In embodiments, L³ is a

bond, —NH—, —NR²⁹—, —S—, —O—, —C(O)—, —NHC(O)—, —C(O)NH—, —NHC(O)NH—, —NHC(NH)NH—, —C(S)—, R²⁹-substituted or unsubstituted C₁-C₂₀ alkylene, R²⁹-substituted or unsubstituted 2 to 20 membered heteroalkylene, R²⁹-substituted or unsubstituted C₃-C₈ cycloalkylene, R²⁹-substituted or unsubstituted 3 to 8 membered heterocycloalkylene, R²⁹-substituted or unsubstituted C₆-C₁₀ arylene, or R²⁹-substituted or unsubstituted 5 to 10 membered heteroarylene. In embodiments, L³ is a bond. In embodiments, L³ is —NH—. In embodiments, L³ is —NR²⁹—. In embodiments, L³ is —S—. In embodiments, L³ is —O—. In embodiments, L³ is —C(O)—. In embodiments, L³ is —NHC(O)—. In embodiments, L³ is —C(O)NH—. In embodiments, L³ is —NHC(O)NH—. In embodiments, L³ is —NHC(NH)NH—. In embodiments, L³ is —C(S)—. In embodiments, L³ is R²⁹-substituted or unsubstituted C₁-C₂₀ alkylene. In embodiments, L³ is R²⁹-substituted or unsubstituted 2 to 20 membered heteroalkylene. In embodiments, L³ is R²⁹-substituted or unsubstituted C₃-C₈ cycloalkylene. In embodiments, L³ is R²⁹-substituted or unsubstituted 3 to 8 membered heterocycloalkylene. In embodiments, L³ is R²⁹-substituted or unsubstituted C₆-C₁₀ arylene. In embodiments, L³ is R²⁹-substituted or unsubstituted 5 to 10 membered heteroarylene. In embodiments, L³ is R²⁹-substituted C₁-C₂₀ alkylene. In embodiments, L³ is R²⁹-substituted 2 to 20 membered heteroalkylene. In embodiments, L³ is R²⁹-substituted C₃-C₈ cycloalkylene. In embodiments, L³ is R²⁹-substituted 3 to 8 membered heterocycloalkylene. In embodiments, L³ is R²⁹-substituted C₆-C₁₀ arylene. In embodiments, L³ is R²⁹-substituted 5 to 10 membered heteroarylene. In embodiments, L³ is unsubstituted C₁-C₂₀ alkylene. In embodiments, L³ is unsubstituted 2 to 20 membered heteroalkylene. In embodiments, L³ is unsubstituted C₃-C₈ cycloalkylene. In embodiments, L³ is unsubstituted 3 to 8 membered heterocycloalkylene. In embodiments, L³ is unsubstituted C₆-C₁₀ arylene. In embodiments, L³ is unsubstituted 5 to 10 membered heteroarylene. In embodiments, L³ is R²⁹-substituted C₁-C₁₅ alkylene. In embodiments, L³ is R²⁹-substituted 2 to 15 membered heteroalkylene. In embodiments, L³ is R²⁹-substituted C₃-C₆ cycloalkylene. In embodiments, L³ is R²⁹-substituted 3 to 6 membered heterocycloalkylene. In embodiments, L³ is R²⁹-substituted phenylene. In embodiments, L³ is R²⁹-substituted 5 to 6 membered heteroarylene. In embodiments, L³ is unsubstituted C₁-C₁₅ alkylene. In embodiments, L³ is unsubstituted 2 to 15 membered heteroalkylene. In embodiments, L³ is unsubstituted C₃-C₆ cycloalkylene. In embodiments, L³ is unsubstituted 3 to 6 membered heterocycloalkylene. In embodiments, L³ is unsubstituted phenylene. In embodiments, L³ is unsubstituted 5 to 6 membered heteroarylene. In embodiments, L³ is R²⁹-substituted C₁-C₁₀ alkylene. In embodiments, L³ is R²⁹-substituted 2 to 10 membered heteroalkylene. In embodiments, L³ is R²⁹-substituted C₄-C₆ cycloalkylene. In embodiments, L³ is R²⁹-substituted 4 to 6 membered heterocycloalkylene. In embodiments, L³ is R²⁹-substituted phenylene. In embodiments, L³ is R²⁹-substituted 5 membered heteroarylene. In embodiments, L³ is R²⁹-substituted C₁-C₈ alkylene. In embodiments, L³ is R²⁹-substituted 2 to 8 membered heteroalkylene. In embodiments, L³ is R²⁹-substituted C₅-C₆ cycloalkylene. In embodiments, L³ is R²⁹-substituted 5 to 6 membered heterocycloalkylene. In embodiments, L³ is R²⁹-substituted 6 membered heteroarylene. In embodiments, L³ is R²⁹-substituted C₁-C₆ alkylene. In embodiments, L³ is R²⁹-substituted 2 to 6 membered heteroalkylene. In embodiments, L³ is R²⁹-substituted C₆-C₂₀ alkylene. In embodiments, L³ is R²⁹-substituted 6 to 20 membered heteroalkylene. In embodiments, L³ is unsubstituted C₁-C₁₀ alkylene. In embodiments, L³ is unsubstituted 2 to 10 membered heteroalkylene. In embodiments, L³ is unsubstituted C₄-C₆ cycloalkylene. In embodiments, L³ is unsubstituted 4 to 6 membered heterocycloalkylene. In embodiments, L³ is unsubstituted phenylene. In embodiments, L³ is unsubstituted 5 membered heteroarylene. In embodiments, L³ is unsubstituted C₁-C₈ alkylene. In embodiments, L³ is unsubstituted 2 to 8 membered heteroalkylene. In embodiments, L³ is unsubstituted C₅-C₆ cycloalkylene. In embodiments, L³ is unsubstituted 5 to 6 membered heterocycloalkylene. In embodiments, L³ is unsubstituted 6 membered heteroarylene. In embodiments, L³ is unsubstituted C₁-C₆ alkylene. In embodiments, L³ is unsubstituted 2 to 6 membered heteroalkylene. In embodiments, L³ is unsubstituted C₆-C₂₀ alkylene. In embodiments, L³ is unsubstituted 6 to 20 membered heteroalkylene.

R²⁹ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁰-substituted or unsubstituted alkyl, R³⁰-substituted or unsubstituted heteroalkyl, R³⁰-substituted or unsubstituted cycloalkyl, R³⁰-substituted or unsubstituted heterocycloalkyl, R³⁰-substituted or unsubstituted aryl, or R³⁰-substituted or unsubstituted heteroaryl.

In embodiments, R²⁹ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R³⁰-substituted or unsubstituted alkyl (e.g., C₁-C₈, C₁-C₆, C₁-C₄, or C₁-C₂), R³⁰-substituted or unsubstituted heteroalkyl (e.g., 2 to 8 membered, 2 to 6 membered, 4 to 6 membered, 2 to 3 membered, or 4 to 5 membered), R³⁰-substituted or unsubstituted cycloalkyl (e.g., C₃-C₈, C₃-C₆, C₄-C₆, or C₅-C₆), R³⁰-substituted or unsubstituted heterocycloalkyl (e.g., 3 to 8 membered, 3 to 6 membered, 4 to 6 membered, 4 to 5 membered, or 5 to 6 membered), R³⁰-substituted or unsubstituted aryl (e.g., C₆-C₁₀ or phenyl), or R³⁰-substituted or unsubstituted heteroaryl (e.g., 5 to 10 membered, 5 to 9 membered, or 5 to 6 membered).

In embodiments, R²⁹ is independently —NH₂. In embodiments, R²⁹ is independently —OH. In embodiments, R²⁹ is independently halogen. In embodiments, R²⁹ is independently —CN. In embodiments, R²⁹ is independently oxo. In embodiments, R²⁹ is independently —CF₃. In embodiments, R²⁹ is independently —COOH. In embodiments, R²⁹ is independently —CONH₂. In embodiments, R²⁹ is independently —NO₂. In embodiments, R²⁹ is independently —SH. In embodiments, R²⁹ is independently —SO₃H. In embodiments, R²⁹ is independently —SO₄H. In embodiments, R²⁹ is independently —SO₂NH₂. In embodiments, R²⁹ is independently —NHNH₂. In embodiments, R²⁹ is independently —ONH₂. In embodiments, R²⁹ is independently —NHC═(O)NHNH₂. In embodiments, R²⁹ is independently —NHC═(O) NH₂. In embodiments, R²⁹ is independently —NHSO₂H. In embodiments, R²⁹ is independently —NHC═(O)H. In embodiments, R²⁹ is independently —NHC(O)—OH. In embodiments, R²⁹ is independently —NHOH. In embodiments, R²⁹ is independently —OCF₃. In embodiments, R²⁹ is independently —OCHF₂. In embodiments, R²⁹ is independently —CCl₃. In embodiments, R²⁹ is independently —CBr₃. In embodiments, R²⁹ is independently —CI₃. In embodiments, R²⁹ is independently —F. In embodiments, R²⁹ is independently —Cl. In embodiments, R²⁹ is independently —Br. In embodiments, R²⁹ is independently —I. In embodiments, R²⁹ is independently R³⁰-substituted C₁-C₄ alkyl. In embodiments, R²⁹ is independently R³⁰-substituted 2 to 4 membered heteroalkyl. In embodiments, R²⁹ is independently R³⁰-substituted C₃-C₆ cycloalkyl. In embodiments, R²⁹ is independently R³⁰-substituted 3 to 6 membered heterocycloalkyl. In embodiments, R²⁹ is independently R³⁰-substituted phenyl. In embodiments, R²⁹ is independently R³⁰-substituted 5 to 6 membered heteroaryl. In embodiments, R²⁹ is independently unsubstituted C₁-C₄ alkyl. In embodiments, R²⁹ is independently unsubstituted 2 to 4 membered heteroalkyl. In embodiments, R²⁹ is independently unsubstituted C₃-C₆ cycloalkyl. In embodiments, R²⁹ is independently unsubstituted 3 to 6 membered heterocycloalkyl. In embodiments, R²⁹ is independently unsubstituted phenyl. In embodiments, R²⁹ is independently unsubstituted 5 to 6 membered heteroaryl.

R³⁰ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl.

In embodiments, R³⁰ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered).

In embodiments, R³⁰ is independently —NH₂. In embodiments, R³⁰ is independently —OH. In embodiments, R³⁰ is independently halogen. In embodiments, R³⁰ is independently —CN. In embodiments, R³⁰ is independently oxo. In embodiments, R³⁰ is independently —CF₃. In embodiments, R³⁰ is independently —COOH. In embodiments, R³⁰ is independently —CONH₂. In embodiments, R³⁰ is independently —NO₂. In embodiments, R³⁰ is independently —SH. In embodiments, R³⁰ is independently —SO₃H. In embodiments, R³⁰ is independently —SO₄H. In embodiments, R³⁰ is independently —SO₂NH₂. In embodiments, R³⁰ is independently —NHNH₂. In embodiments, R³⁰ is independently —ONH₂. In embodiments, R³⁰ is independently —NHC═(O)NHNH₂. In embodiments, R³⁰ is independently —NHC═(O) NH₂. In embodiments, R³⁰ is independently —NHSO₂H. In embodiments, R³⁰ is independently —NHC═(O)H. In embodiments, R³⁰ is independently —NHC(O)—OH. In embodiments, R³⁰ is independently —NHOH. In embodiments, R³⁰ is independently —OCF₃. In embodiments, R³⁰ is independently —OCHF₂. In embodiments, R³⁰ is independently —CCl₃. In embodiments, R³⁰ is independently —CBr₃. In embodiments, R³⁰ is independently —CI₃. In embodiments, R³⁰ is independently —F. In embodiments, R³⁰ is independently —Cl. In embodiments, R³⁰ is independently —Br. In embodiments, R³⁰ is independently —I. In embodiments, R³⁰ is independently R³¹-substituted C₁-C₄ alkyl. In embodiments, R³⁰ is independently R³¹-substituted 2 to 4 membered heteroalkyl. In embodiments, R³⁰ is independently R³¹-substituted C₃-C₆ cycloalkyl. In embodiments, R³⁰ is independently R³¹-substituted 3 to 6 membered heterocycloalkyl. In embodiments, R³⁰ is independently R³¹-substituted phenyl. In embodiments, R³⁰ is independently R³¹-substituted 5 to 6 membered heteroaryl. In embodiments, R³⁰ is independently unsubstituted C₁-C₄ alkyl. In embodiments, R³⁰ is independently unsubstituted 2 to 4 membered heteroalkyl. In embodiments, R³⁰ is independently unsubstituted C₃-C₆ cycloalkyl. In embodiments, R³⁰ is independently unsubstituted 3 to 6 membered heterocycloalkyl. In embodiments, R³⁰ is independently unsubstituted phenyl. In embodiments, R³⁰ is independently unsubstituted 5 to 6 membered heteroaryl.

In embodiments, L⁴ is independently a bond, R³²-substituted or unsubstituted alkylene, R³²-substituted or unsubstituted heteroalkylene, R³²-substituted or unsubstituted cycloalkylene, R³²-substituted or unsubstituted heterocycloalkylene, R³²-substituted or unsubstituted arylene, or R³²-substituted or unsubstituted heteroarylene.

In embodiments, L⁴ is a bond, —NH—, —NR³²—, —S—, —O—, —C(O)—, —NHC(O)—, —C(O)NH—, —NHC(O)NH—, —NHC(NH)NH—, —C(S)—, R³²-substituted or unsubstituted C₁-C₂₀ alkylene, R³²-substituted or unsubstituted 2 to 20 membered heteroalkylene, R³²-substituted or unsubstituted C₃-C₈ cycloalkylene, R³²-substituted or unsubstituted 3 to 8 membered heterocycloalkylene, R³²-substituted or unsubstituted C₆-C₁₀ arylene, or R³²-substituted or unsubstituted 5 to 10 membered heteroarylene. In embodiments, L⁴ is a bond. In embodiments, L⁴ is —NH—. In embodiments, L⁴ is —NR³²—. In embodiments, L⁴ is —S—. In embodiments, L⁴ is —O—. In embodiments, L⁴ is —C(O)—. In embodiments, L⁴ is —NHC(O)—. In embodiments, L⁴ is —C(O)NH—. In embodiments, L⁴ is —NHC(O)NH—. In embodiments, L⁴ is —NHC(NH)NH—. In embodiments, L⁴ is —C(S)—. In embodiments, L⁴ is R³²-substituted or unsubstituted C₁-C₂₀ alkylene. In embodiments, L⁴ is R³²-substituted or unsubstituted 2 to 20 membered heteroalkylene. In embodiments, L⁴ is R³²-substituted or unsubstituted C₃-C₈ cycloalkylene. In embodiments, L⁴ is R³²-substituted or unsubstituted 3 to 8 membered heterocycloalkylene. In embodiments, L⁴ is R³²-substituted or unsubstituted C₆-C₁₀ arylene. In embodiments, L⁴ is R³²-substituted or unsubstituted 5 to 10 membered heteroarylene. In embodiments, L⁴ is R³²-substituted C₁-C₂₀ alkylene. In embodiments, L⁴ is R³²-substituted 2 to 20 membered heteroalkylene. In embodiments, L⁴ is R³²-substituted C₃-C₈ cycloalkylene. In embodiments, L⁴ is R³²-substituted 3 to 8 membered heterocycloalkylene. In embodiments, L⁴ is R³²-substituted C₆-C₁₀ arylene. In embodiments, L⁴ is R³²-substituted 5 to 10 membered heteroarylene. In embodiments, L⁴ is unsubstituted C₁-C₂₀ alkylene. In embodiments, L⁴ is unsubstituted 2 to 20 membered heteroalkylene. In embodiments, L⁴ is unsubstituted C₃-C₈ cycloalkylene. In embodiments, L⁴ is unsubstituted 3 to 8 membered heterocycloalkylene. In embodiments, L⁴ is unsubstituted C₆-C₁₀ arylene. In embodiments, L⁴ is unsubstituted 5 to 10 membered heteroarylene. In embodiments, L⁴ is R³²-substituted C₁-C₁₅ alkylene. In embodiments, L⁴ is R³²-substituted 2 to 15 membered heteroalkylene. In embodiments, L⁴ is R³²-substituted C₃-C₆ cycloalkylene. In embodiments, L⁴ is R³²-substituted 3 to 6 membered heterocycloalkylene. In embodiments, L⁴ is R³²-substituted phenylene. In embodiments, L⁴ is R³²-substituted 5 to 6 membered heteroarylene. In embodiments, L⁴ is unsubstituted C₁-C₁₅ alkylene. In embodiments, L⁴ is unsubstituted 2 to 15 membered heteroalkylene. In embodiments, L⁴ is unsubstituted C₃-C₆ cycloalkylene. In embodiments, L⁴ is unsubstituted 3 to 6 membered heterocycloalkylene. In embodiments, L⁴ is unsubstituted phenylene. In embodiments, L⁴ is unsubstituted 5 to 6 membered heteroarylene. In embodiments, L⁴ is R³²-substituted C₁-C₁₀ alkylene. In embodiments, L⁴ is R³²-substituted 2 to 10 membered heteroalkylene. In embodiments, L⁴ is R³²-substituted C₄-C₆ cycloalkylene. In embodiments, L⁴ is R³²-substituted 4 to 6 membered heterocycloalkylene. In embodiments, L⁴ is R³²-substituted phenylene. In embodiments, L⁴ is R³²-substituted 5 membered heteroarylene. In embodiments, L⁴ is R³²-substituted C₁-C₈ alkylene. In embodiments, L⁴ is R³²-substituted 2 to 8 membered heteroalkylene. In embodiments, L⁴ is R³²-substituted C₅-C₆ cycloalkylene. In embodiments, L⁴ is R³²-substituted 5 to 6 membered heterocycloalkylene. In embodiments, L⁴ is R³²-substituted 6 membered heteroarylene. In embodiments, L⁴ is R³²-substituted C₁-C₆ alkylene. In embodiments, L⁴ is R³²-substituted 2 to 6 membered heteroalkylene. In embodiments, L⁴ is R³²-substituted C₆-C₂₀ alkylene. In embodiments, L⁴ is R³²-substituted 6 to 20 membered heteroalkylene. In embodiments, L⁴ is unsubstituted C₁-C₁₀ alkylene. In embodiments, L⁴ is unsubstituted 2 to 10 membered heteroalkylene. In embodiments, L⁴ is unsubstituted C₄-C₆ cycloalkylene. In embodiments, L⁴ is unsubstituted 4 to 6 membered heterocycloalkylene. In embodiments, L⁴ is unsubstituted phenylene. In embodiments, L⁴ is unsubstituted 5 membered heteroarylene. In embodiments, L⁴ is unsubstituted C₁-C₈ alkylene. In embodiments, L⁴ is unsubstituted 2 to 8 membered heteroalkylene. In embodiments, L⁴ is unsubstituted C₅-C₆ cycloalkylene. In embodiments, L⁴ is unsubstituted 5 to 6 membered heterocycloalkylene. In embodiments, L⁴ is unsubstituted 6 membered heteroarylene. In embodiments, L⁴ is unsubstituted C₁-C₆ alkylene. In embodiments, L⁴ is unsubstituted 2 to 6 membered heteroalkylene. In embodiments, L⁴ is unsubstituted C₆-C₂₀ alkylene. In embodiments, L⁴ is unsubstituted 6 to 20 membered heteroalkylene.

R³² is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³³-substituted or unsubstituted alkyl, R³³-substituted or unsubstituted heteroalkyl, R³³-substituted or unsubstituted cycloalkyl, R³³-substituted or unsubstituted heterocycloalkyl, R³³-substituted or unsubstituted aryl, or R³³-substituted or unsubstituted heteroaryl.

In embodiments, R³² is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R³³-substituted or unsubstituted alkyl (e.g., C₁-C₈, C₁-C₆, C₁-C₄, or C₁-C₂), R³³-substituted or unsubstituted heteroalkyl (e.g., 2 to 8 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 8 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered).

In embodiments, R³² is independently —NH₂. In embodiments, R³² is independently —OH. In embodiments, R³² is independently halogen. In embodiments, R³² is independently —CN. In embodiments, R³² is independently oxo. In embodiments, R³² is independently —CF₃. In embodiments, R³² is independently —COOH. In embodiments, R³² is independently —CONH₂. In embodiments, R³² is independently —NO₂. In embodiments, R³² is independently —SH. In embodiments, R³² is independently —SO₃H. In embodiments, R³² is independently —SO₄H. In embodiments, R³² is independently —SO₂NH₂. In embodiments, R³² is independently —NHNH₂. In embodiments, R³² is independently —ONH₂. In embodiments, R³² is independently —NHC═(O)NHNH₂. In embodiments, R³² is independently —NHC═(O) NH₂. In embodiments, R³² is independently —NHSO₂H. In embodiments, R³² is independently —NHC═(O)H. In embodiments, R³² is independently —NHC(O)—OH. In embodiments, R³² is independently —NHOH. In embodiments, R³² is independently —OCF₃. In embodiments, R³² is independently —OCHF₂. In embodiments, R³² is independently —CCl₃. In embodiments, R³² is independently —CBr₃. In embodiments, R³² is independently —CI₃. In embodiments, R³² is independently —F. In embodiments, R³² is independently —Cl. In embodiments, R³² is independently —Br. In embodiments, R³² is independently —I. In embodiments, R³² is independently R³³-substituted C₁-C₄ alkyl. In embodiments, R³² is independently R³³-substituted 2 to 4 membered heteroalkyl. In embodiments, R³² is independently R³³-substituted C₃-C₆ cycloalkyl. In embodiments, R³² is independently R³³-substituted 3 to 6 membered heterocycloalkyl. In embodiments, R³² is independently R³³-substituted phenyl. In embodiments, R³² is independently R³³-substituted 5 to 6 membered heteroaryl. In embodiments, R³² is independently unsubstituted C₁-C₄ alkyl. In embodiments, R³² is independently unsubstituted 2 to 4 membered heteroalkyl. In embodiments, R³² is independently unsubstituted C₃-C₆ cycloalkyl. In embodiments, R³² is independently unsubstituted 3 to 6 membered heterocycloalkyl. In embodiments, R³² is independently unsubstituted phenyl. In embodiments, R³² is independently unsubstituted 5 to 6 membered heteroaryl.

R³³ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl.

In embodiments, R³³ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R³⁴-substituted or unsubstituted alkyl (e.g., C₁-C₈, C₁-C₆, C₁-C₄, or C₁-C₂), R³⁴-substituted or unsubstituted heteroalkyl (e.g., 2 to 8 membered, 2 to 6 membered, 4 to 6 membered, 2 to 3 membered, or 4 to 5 membered), R³⁴-substituted or unsubstituted cycloalkyl (e.g., C₃-C₈, C₃-C₆, C₄-C₆, or C₅-C₆), R³⁴-substituted or unsubstituted heterocycloalkyl (e.g., 3 to 8 membered, 3 to 6 membered, 4 to 6 membered, 4 to 5 membered, or 5 to 6 membered), R³⁴-substituted or unsubstituted aryl (e.g., C₆-C₁₀ or phenyl), or R³⁴-substituted or unsubstituted heteroaryl (e.g., 5 to 10 membered, 5 to 9 membered, or 5 to 6 membered).

In embodiments, R³³ is independently —NH₂. In embodiments, R³³ is independently —OH. In embodiments, R³³ is independently halogen. In embodiments, R³³ is independently —CN. In embodiments, R³³ is independently oxo. In embodiments, R³³ is independently —CF₃. In embodiments, R³³ is independently —COOH. In embodiments, R³³ is independently —CONH₂. In embodiments, R³³ is independently —NO₂. In embodiments, R³³ is independently —SH. In embodiments, R³³ is independently —SO₃H. In embodiments, R³³ is independently —SO₄H. In embodiments, R³³ is independently —SO₂NH₂. In embodiments, R³³ is independently —NHNH₂. In embodiments, R³³ is independently —ONH₂. In embodiments, R³³ is independently —NHC═(O)NHNH₂. In embodiments, R³³ is independently —NHC═(O) NH₂. In embodiments, R³³ is independently —NHSO₂H. In embodiments, R³³ is independently —NHC═(O)H. In embodiments, R³³ is independently —NHC(O)—OH. In embodiments, R³³ is independently —NHOH. In embodiments, R³³ is independently —OCF₃. In embodiments, R³³ is independently —OCHF₂. In embodiments, R³³ is independently —CCl₃. In embodiments, R³³ is independently —CBr₃. In embodiments, R³³ is independently —CI₃. In embodiments, R³³ is independently —F. In embodiments, R³³ is independently —Cl. In embodiments, R³³ is independently —Br. In embodiments, R³³ is independently —I. In embodiments, R³³ is independently R³⁴-substituted C₁-C₄ alkyl. In embodiments, R³³ is independently R³⁴-substituted 2 to 4 membered heteroalkyl. In embodiments, R³³ is independently R³⁴-substituted C₃-C₆ cycloalkyl. In embodiments, R³³ is independently R³⁴-substituted 3 to 6 membered heterocycloalkyl. In embodiments, R³³ is independently R³⁴-substituted phenyl. In embodiments, R³³ is independently R³⁴-substituted 5 to 6 membered heteroaryl. In embodiments, R³³ is independently unsubstituted C₁-C₄ alkyl. In embodiments, R³³ is independently unsubstituted 2 to 4 membered heteroalkyl. In embodiments, R³³ is independently unsubstituted C₃-C₆ cycloalkyl. In embodiments, R³³ is independently unsubstituted 3 to 6 membered heterocycloalkyl. In embodiments, R³³ is independently unsubstituted phenyl. In embodiments, R³³ is independently unsubstituted 5 to 6 membered heteroaryl.

In embodiments, L⁵ is independently a bond, R³⁵-substituted or unsubstituted alkylene, R³⁵-substituted or unsubstituted heteroalkylene, R³⁵-substituted or unsubstituted cycloalkylene, R³⁵-substituted or unsubstituted heterocycloalkylene, R³⁵-substituted or unsubstituted arylene, or R³⁵-substituted or unsubstituted heteroarylene.

In embodiments, L⁵ is a

bond, —NH—, —NR³⁵—, —S—, —O—, —C(O)—, —NHC(O)—, —C(O)NH—, —NHC(O)NH—, —NHC(NH)NH—, —C(S)—, R³⁵-substituted or unsubstituted C₁-C₂₀ alkylene, R³⁵-substituted or unsubstituted 2 to 20 membered heteroalkylene, R³⁵-substituted or unsubstituted C₃-C₈ cycloalkylene, R³⁵-substituted or unsubstituted 3 to 8 membered heterocycloalkylene, R³⁵-substituted or unsubstituted C₆-C₁₀ arylene, or R³⁵-substituted or unsubstituted 5 to 10 membered heteroarylene. In embodiments, L⁵ is a bond. In embodiments, L⁵ is —NH—. In embodiments, L⁵ is —NR³⁵—. In embodiments, L⁵ is —S—. In embodiments, L⁵ is —O—. In embodiments, L⁵ is —C(O)—. In embodiments, L⁵ is —NHC(O)—. In embodiments, L⁵ is —C(O)NH—. In embodiments, L⁵ is —NHC(O)NH—. In embodiments, L⁵ is —NHC(NH)NH—. In embodiments, L⁵ is —C(S)—. In embodiments, L⁵ is R³⁵-substituted or unsubstituted C₁-C₂₀ alkylene. In embodiments, L⁵ is R³⁵-substituted or unsubstituted 2 to 20 membered heteroalkylene. In embodiments, L⁵ is R³⁵-substituted or unsubstituted C₃-C₈ cycloalkylene. In embodiments, L⁵ is R³⁵-substituted or unsubstituted 3 to 8 membered heterocycloalkylene. In embodiments, L⁵ is R³⁵-substituted or unsubstituted C₆-C₁₀ arylene. In embodiments, L⁵ is R³⁵-substituted or unsubstituted 5 to 10 membered heteroarylene. In embodiments, L⁵ is R³⁵-substituted C₁-C₂₀ alkylene. In embodiments, L⁵ is R³⁵-substituted 2 to 20 membered heteroalkylene. In embodiments, L⁵ is R³⁵-substituted C₃-C₈ cycloalkylene. In embodiments, L⁵ is R³⁵-substituted 3 to 8 membered heterocycloalkylene. In embodiments, L⁵ is R³⁵-substituted C₆-C₁₀ arylene. In embodiments, L⁵ is R³⁵-substituted 5 to 10 membered heteroarylene. In embodiments, L⁵ is unsubstituted C₁-C₂₀ alkylene. In embodiments, L⁵ is unsubstituted 2 to 20 membered heteroalkylene. In embodiments, L⁵ is unsubstituted C₃-C₈ cycloalkylene. In embodiments, L⁵ is unsubstituted 3 to 8 membered heterocycloalkylene. In embodiments, L⁵ is unsubstituted C₆-C₁₀ arylene. In embodiments, L⁵ is unsubstituted 5 to 10 membered heteroarylene. In embodiments, L⁵ is R³⁵-substituted C₁-C₁₅ alkylene. In embodiments, L⁵ is R³⁵-substituted 2 to 15 membered heteroalkylene. In embodiments, L⁵ is R³⁵-substituted C₃-C₆ cycloalkylene. In embodiments, L⁵ is R³⁵-substituted 3 to 6 membered heterocycloalkylene. In embodiments, L⁵ is R³⁵-substituted phenylene. In embodiments, L⁵ is R³⁵-substituted 5 to 6 membered heteroarylene. In embodiments, L⁵ is unsubstituted C₁-C₁₅ alkylene. In embodiments, L⁵ is unsubstituted 2 to 15 membered heteroalkylene. In embodiments, L⁵ is unsubstituted C₃-C₆ cycloalkylene. In embodiments, L⁵ is unsubstituted 3 to 6 membered heterocycloalkylene. In embodiments, L⁵ is unsubstituted phenylene. In embodiments, L⁵ is unsubstituted 5 to 6 membered heteroarylene. In embodiments, L⁵ is R³⁵-substituted C₁-C₁₀ alkylene. In embodiments, L⁵ is R³⁵-substituted 2 to 10 membered heteroalkylene. In embodiments, L⁵ is R³⁵-substituted C₄-C₆ cycloalkylene. In embodiments, L⁵ is R³⁵-substituted 4 to 6 membered heterocycloalkylene. In embodiments, L⁵ is R³⁵-substituted phenylene. In embodiments, L⁵ is R³⁵-substituted 5 membered heteroarylene. In embodiments, L⁵ is R³⁵-substituted C₁-C₈ alkylene. In embodiments, L⁵ is R³⁵-substituted 2 to 8 membered heteroalkylene. In embodiments, L⁵ is R³⁵-substituted C₅-C₆ cycloalkylene. In embodiments, L⁵ is R³⁵-substituted 5 to 6 membered heterocycloalkylene. In embodiments, L⁵ is R³⁵-substituted 6 membered heteroarylene. In embodiments, L⁵ is R³⁵-substituted C₁-C₆ alkylene. In embodiments, L⁵ is R³⁵-substituted 2 to 6 membered heteroalkylene. In embodiments, L⁵ is R³⁵-substituted C₆-C₂₀ alkylene. In embodiments, L⁵ is R³⁵-substituted 6 to 20 membered heteroalkylene. In embodiments, L⁵ is unsubstituted C₁-C₁₀ alkylene. In embodiments, L⁵ is unsubstituted 2 to 10 membered heteroalkylene. In embodiments, L⁵ is unsubstituted C₄-C₆ cycloalkylene. In embodiments, L⁵ is unsubstituted 4 to 6 membered heterocycloalkylene. In embodiments, L⁵ is unsubstituted phenylene. In embodiments, L⁵ is unsubstituted 5 membered heteroarylene. In embodiments, L⁵ is unsubstituted C₁-C₈ alkylene. In embodiments, L⁵ is unsubstituted 2 to 8 membered heteroalkylene. In embodiments, L⁵ is unsubstituted C₅-C₆ cycloalkylene. In embodiments, L⁵ is unsubstituted 5 to 6 membered heterocycloalkylene. In embodiments, L⁵ is unsubstituted 6 membered heteroarylene. In embodiments, L⁵ is unsubstituted C₁-C₆ alkylene. In embodiments, L⁵ is unsubstituted 2 to 6 membered heteroalkylene. In embodiments, L⁵ is unsubstituted C₆-C₂₀ alkylene. In embodiments, L⁵ is unsubstituted 6 to 20 membered heteroalkylene.

R³⁵ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁶-substituted or unsubstituted alkyl, R³⁶-substituted or unsubstituted heteroalkyl, R³⁶-substituted or unsubstituted cycloalkyl, R³⁶-substituted or unsubstituted heterocycloalkyl, R³⁶-substituted or unsubstituted aryl, or R³⁶-substituted or unsubstituted heteroaryl.

In embodiments, R³⁵ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R³⁶-substituted or unsubstituted alkyl (e.g., C₁-C₈, C₁-C₆, C₁-C₄, or C₁-C₂), R³⁶-substituted or unsubstituted heteroalkyl (e.g., 2 to 8 membered, 2 to 6 membered, 4 to 6 membered, 2 to 3 membered, or 4 to 5 membered), R³⁶-substituted or unsubstituted cycloalkyl (e.g., C₃-C₈, C₃-C₆, C₄-C₆, or C₅-C₆), R³⁶-substituted or unsubstituted heterocycloalkyl (e.g., 3 to 8 membered, 3 to 6 membered, 4 to 6 membered, 4 to 5 membered, or 5 to 6 membered), R³⁶-substituted or unsubstituted aryl (e.g., C₆-C₁₀ or phenyl), or R³⁶-substituted or unsubstituted heteroaryl (e.g., 5 to 10 membered, 5 to 9 membered, or 5 to 6 membered).

In embodiments, R³⁵ is independently —NH₂. In embodiments, R³⁵ is independently —OH. In embodiments, R³⁵ is independently halogen. In embodiments, R³⁵ is independently —CN. In embodiments, R³⁵ is independently oxo. In embodiments, R³⁵ is independently —CF₃. In embodiments, R³⁵ is independently —COOH. In embodiments, R³⁵ is independently —CONH₂. In embodiments, R³⁵ is independently —NO₂. In embodiments, R³⁵ is independently —SH. In embodiments, R³⁵ is independently —SO₃H. In embodiments, R³⁵ is independently —SO₄H. In embodiments, R³⁵ is independently —SO₂NH₂. In embodiments, R³⁵ is independently —NHNH₂. In embodiments, R³⁵ is independently —ONH₂. In embodiments, R³⁵ is independently —NHC═(O)NHNH₂. In embodiments, R³⁵ is independently —NHC═(O) NH₂. In embodiments, R³⁵ is independently —NHSO₂H. In embodiments, R³⁵ is independently —NHC═(O)H. In embodiments, R³⁵ is independently —NHC(O)—OH. In embodiments, R³⁵ is independently —NHOH. In embodiments, R³⁵ is independently —OCF₃. In embodiments, R³⁵ is independently —OCHF₂. In embodiments, R³⁵ is independently —CCl₃. In embodiments, R³⁵ is independently —CBr₃. In embodiments, R³⁵ is independently —CI₃. In embodiments, R³⁵ is independently —F. In embodiments, R³⁵ is independently —Cl. In embodiments, R³⁵ is independently —Br. In embodiments, R³⁵ is independently —I. In embodiments, R³⁵ is independently R³⁶-substituted C₁-C₄ alkyl. In embodiments, R³⁵ is independently R³⁶-substituted 2 to 4 membered heteroalkyl. In embodiments, R³⁵ is independently R³⁶-substituted C₃-C₆ cycloalkyl. In embodiments, R³⁵ is independently R³⁶-substituted 3 to 6 membered heterocycloalkyl. In embodiments, R³⁵ is independently R³⁶-substituted phenyl. In embodiments, R³⁵ is independently R³⁶-substituted 5 to 6 membered heteroaryl. In embodiments, R³⁵ is independently unsubstituted C₁-C₄ alkyl. In embodiments, R³⁵ is independently unsubstituted 2 to 4 membered heteroalkyl. In embodiments, R³⁵ is independently unsubstituted C₃-C₆ cycloalkyl. In embodiments, R³⁵ is independently unsubstituted 3 to 6 membered heterocycloalkyl. In embodiments, R³⁵ is independently unsubstituted phenyl. In embodiments, R³⁵ is independently unsubstituted 5 to 6 membered heteroaryl.

R³⁶ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁷-substituted or unsubstituted alkyl, R³⁷-substituted or unsubstituted heteroalkyl, R³⁷-substituted or unsubstituted cycloalkyl, R³⁷-substituted or unsubstituted heterocycloalkyl, R³⁷-substituted or unsubstituted aryl, or R³⁷-substituted or unsubstituted heteroaryl.

In embodiments, R³⁶ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered).

In embodiments, R³⁶ is independently —NH₂. In embodiments, R³⁶ is independently —OH. In embodiments, R³⁶ is independently halogen. In embodiments, R³⁶ is independently —CN. In embodiments, R³⁶ is independently oxo. In embodiments, R³⁶ is independently —CF₃. In embodiments, R³⁶ is independently —COOH. In embodiments, R³⁶ is independently —CONH₂. In embodiments, R³⁶ is independently —NO₂. In embodiments, R³⁶ is independently —SH. In embodiments, R³⁶ is independently —SO₃H. In embodiments, R³⁶ is independently —SO₄H. In embodiments, R³⁶ is independently —SO₂NH₂. In embodiments, R³⁶ is independently —NHNH₂. In embodiments, R³⁶ is independently —ONH₂. In embodiments, R³⁶ is independently —NHC═(O)NHNH₂. In embodiments, R³⁶ is independently —NHC═(O) NH₂. In embodiments, R³⁶ is independently —NHSO₂H. In embodiments, R³⁶ is independently —NHC═(O)H. In embodiments, R³⁶ is independently —NHC(O)—OH. In embodiments, R³⁶ is independently —NHOH. In embodiments, R³⁶ is independently —OCF₃. In embodiments, R³⁶ is independently —OCHF₂. In embodiments, R³⁶ is independently —CCl₃. In embodiments, R³⁶ is independently —CBr₃. In embodiments, R³⁶ is independently —CI₃. In embodiments, R³⁶ is independently —F. In embodiments, R³⁶ is independently —Cl. In embodiments, R³⁶ is independently —Br. In embodiments, R³⁶ is independently —I. In embodiments, R³⁶ is independently R³⁷-substituted C₁-C₄ alkyl. In embodiments, R³⁶ is independently R³⁷-substituted 2 to 4 membered heteroalkyl. In embodiments, R³⁶ is independently R³⁷-substituted C₃-C₆ cycloalkyl. In embodiments, R³⁶ is independently R³⁷-substituted 3 to 6 membered heterocycloalkyl. In embodiments, R³⁶ is independently R³⁷-substituted phenyl. In embodiments, R³⁶ is independently R³⁷-substituted 5 to 6 membered heteroaryl. In embodiments, R³⁶ is independently unsubstituted C₁-C₄ alkyl. In embodiments, R³⁶ is independently unsubstituted 2 to 4 membered heteroalkyl. In embodiments, R³⁶ is independently unsubstituted C₃-C₆ cycloalkyl. In embodiments, R³⁶ is independently unsubstituted 3 to 6 membered heterocycloalkyl. In embodiments, R³⁶ is independently unsubstituted phenyl. In embodiments, R³⁶ is independently unsubstituted 5 to 6 membered heteroaryl.

In embodiments, L⁶ is independently a bond, R³⁸-substituted or unsubstituted alkylene, R³⁸-substituted or unsubstituted heteroalkylene, R³⁸-substituted or unsubstituted cycloalkylene, R³⁸-substituted or unsubstituted heterocycloalkylene, R³⁸-substituted or unsubstituted arylene, or R³⁸-substituted or unsubstituted heteroarylene.

In embodiments, L⁶ is a

bond, —NH—, —NR³⁸—, —S—, —O—, —C(O)—, —NHC(O)—, —C(O)NH—, —NHC(O)NH—, —NHC(NH)NH—, —C(S)—, R³⁸-substituted or unsubstituted C₁-C₂₀ alkylene, R³⁸-substituted or unsubstituted 2 to 20 membered heteroalkylene, R³⁸-substituted or unsubstituted C₃-C₈ cycloalkylene, R³⁸-substituted or unsubstituted 3 to 8 membered heterocycloalkylene, R³⁸-substituted or unsubstituted C₆-C₁₀ arylene, or R³⁸-substituted or unsubstituted 5 to 10 membered heteroarylene. In embodiments, L⁶ is a bond. In embodiments, L⁶ is —NH—. In embodiments, L⁶ is —NR³⁸—. In embodiments, L⁶ is —S—. In embodiments, L⁶ is —O—. In embodiments, L⁶ is —C(O)—. In embodiments, L⁶ is —NHC(O)—. In embodiments, L⁶ is —C(O)NH—. In embodiments, L⁶ is —NHC(O)NH—. In embodiments, L⁶ is —NHC(NH)NH—. In embodiments, L⁶ is —C(S)—. In embodiments, L⁶ is R³⁸-substituted or unsubstituted C₁-C₂₀ alkylene. In embodiments, L⁶ is R³⁸-substituted or unsubstituted 2 to 20 membered heteroalkylene. In embodiments, L⁶ is R³⁸-substituted or unsubstituted C₃-C₈ cycloalkylene. In embodiments, L⁶ is R³⁸-substituted or unsubstituted 3 to 8 membered heterocycloalkylene. In embodiments, L⁶ is R³⁸-substituted or unsubstituted C₆-C₁₀ arylene. In embodiments, L⁶ is R³⁸-substituted or unsubstituted 5 to 10 membered heteroarylene. In embodiments, L⁶ is R³⁸-substituted C₁-C₂₀ alkylene. In embodiments, L⁶ is R³⁸-substituted 2 to 20 membered heteroalkylene. In embodiments, L⁶ is R³⁸-substituted C₃-C₈ cycloalkylene. In embodiments, L⁶ is R³⁸-substituted 3 to 8 membered heterocycloalkylene. In embodiments, L⁶ is R³⁸-substituted C₆-C₁₀ arylene. In embodiments, L⁶ is R³⁸-substituted 5 to 10 membered heteroarylene. In embodiments, L⁶ is unsubstituted C₁-C₂₀ alkylene. In embodiments, L⁶ is unsubstituted 2 to 20 membered heteroalkylene. In embodiments, L⁶ is unsubstituted C₃-C₈ cycloalkylene. In embodiments, L⁶ is unsubstituted 3 to 8 membered heterocycloalkylene. In embodiments, L⁶ is unsubstituted C₆-C₁₀ arylene. In embodiments, L⁶ is unsubstituted 5 to 10 membered heteroarylene. In embodiments, L⁶ is R³⁸-substituted C₁-C₁₅ alkylene. In embodiments, L⁶ is R³⁸-substituted 2 to 15 membered heteroalkylene. In embodiments, L⁶ is R³⁸-substituted C₃-C₆ cycloalkylene. In embodiments, L⁶ is R³⁸-substituted 3 to 6 membered heterocycloalkylene. In embodiments, L⁶ is R³⁸-substituted phenylene. In embodiments, L⁶ is R³⁸-substituted 5 to 6 membered heteroarylene. In embodiments, L⁶ is unsubstituted C₁-C₁₅ alkylene. In embodiments, L⁶ is unsubstituted 2 to 15 membered heteroalkylene. In embodiments, L⁶ is unsubstituted C₃-C₆ cycloalkylene. In embodiments, L⁶ is unsubstituted 3 to 6 membered heterocycloalkylene. In embodiments, L⁶ is unsubstituted phenylene. In embodiments, L⁶ is unsubstituted 5 to 6 membered heteroarylene. In embodiments, L⁶ is R³⁸-substituted C₁-C₁₀ alkylene. In embodiments, L⁶ is R³⁸-substituted 2 to 10 membered heteroalkylene. In embodiments, L⁶ is R³⁸-substituted C₄-C₆ cycloalkylene. In embodiments, L⁶ is R³⁸-substituted 4 to 6 membered heterocycloalkylene. In embodiments, L⁶ is R³⁸-substituted phenylene. In embodiments, L⁶ is R³⁸-substituted 5 membered heteroarylene. In embodiments, L⁶ is R³⁸-substituted C₁-C₈ alkylene. In embodiments, L⁶ is R³⁸-substituted 2 to 8 membered heteroalkylene. In embodiments, L⁶ is R³⁸-substituted C₅-C₆ cycloalkylene. In embodiments, L⁶ is R³⁸-substituted 5 to 6 membered heterocycloalkylene. In embodiments, L⁶ is R³⁸-substituted 6 membered heteroarylene. In embodiments, L⁶ is R³⁸-substituted C₁-C₆ alkylene. In embodiments, L⁶ is R³⁸-substituted 2 to 6 membered heteroalkylene. In embodiments, L⁶ is R³⁸-substituted C₆-C₂₀ alkylene. In embodiments, L⁶ is R³⁸-substituted 6 to 20 membered heteroalkylene. In embodiments, L⁶ is unsubstituted C₁-C₁₀ alkylene. In embodiments, L⁶ is unsubstituted 2 to 10 membered heteroalkylene. In embodiments, L⁶ is unsubstituted C₄-C₆ cycloalkylene. In embodiments, L⁶ is unsubstituted 4 to 6 membered heterocycloalkylene. In embodiments, L⁶ is unsubstituted phenylene. In embodiments, L⁶ is unsubstituted 5 membered heteroarylene. In embodiments, L⁶ is unsubstituted C₁-C₈ alkylene. In embodiments, L⁶ is unsubstituted 2 to 8 membered heteroalkylene. In embodiments, L⁶ is unsubstituted C₅-C₆ cycloalkylene. In embodiments, L⁶ is unsubstituted 5 to 6 membered heterocycloalkylene. In embodiments, L⁶ is unsubstituted 6 membered heteroarylene. In embodiments, L⁶ is unsubstituted C₁-C₆ alkylene. In embodiments, L⁶ is unsubstituted 2 to 6 membered heteroalkylene. In embodiments, L⁶ is unsubstituted C₆-C₂₀ alkylene. In embodiments, L⁶ is unsubstituted 6 to 20 membered heteroalkylene.

R³⁸ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁹-substituted or unsubstituted alkyl, R³⁹-substituted or unsubstituted heteroalkyl, R³⁹-substituted or unsubstituted cycloalkyl, R³⁹-substituted or unsubstituted heterocycloalkyl, R³⁹-substituted or unsubstituted aryl, or R³⁹-substituted or unsubstituted heteroaryl.

In embodiments, R³⁸ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R³⁹-substituted or unsubstituted alkyl (e.g., C₁-C₈, C₁-C₆, C₁-C₄, or C₁-C₂), R³⁹-substituted or unsubstituted heteroalkyl (e.g., 2 to 8 membered, 2 to 6 membered, 4 to 6 membered, 2 to 3 membered, or 4 to 5 membered), R³⁹-substituted or unsubstituted cycloalkyl (e.g., C₃-C₈, C₃-C₆, C₄-C₆, or C₅-C₆), R³⁹-substituted or unsubstituted heterocycloalkyl (e.g., 3 to 8 membered, 3 to 6 membered, 4 to 6 membered, 4 to 5 membered, or 5 to 6 membered), R³⁹-substituted or unsubstituted aryl (e.g., C₆-C₁₀ or phenyl), or R³⁹-substituted or unsubstituted heteroaryl (e.g., 5 to 10 membered, 5 to 9 membered, or 5 to 6 membered).

In embodiments, R³⁸ is independently —NH₂. In embodiments, R³⁸ is independently —OH. In embodiments, R³⁸ is independently halogen. In embodiments, R³⁸ is independently —CN. In embodiments, R³⁸ is independently oxo. In embodiments, R³⁸ is independently —CF₃. In embodiments, R³⁸ is independently —COOH. In embodiments, R³⁸ is independently —CONH₂. In embodiments, R³⁸ is independently —NO₂. In embodiments, R³⁸ is independently —SH. In embodiments, R³⁸ is independently —SO₃H. In embodiments, R³⁸ is independently —SO₄H. In embodiments, R³⁸ is independently —SO₂NH₂. In embodiments, R³⁸ is independently —NHNH₂. In embodiments, R³⁸ is independently —ONH₂. In embodiments, R³⁸ is independently —NHC═(O)NHNH₂. In embodiments, R³⁸ is independently —NHC═(O) NH₂. In embodiments, R³⁸ is independently —NHSO₂H. In embodiments, R³⁸ is independently —NHC═(O)H. In embodiments, R³⁸ is independently —NHC(O)—OH. In embodiments, R³⁸ is independently —NHOH. In embodiments, R³⁸ is independently —OCF₃. In embodiments, R³⁸ is independently —OCHF₂. In embodiments, R³⁸ is independently —CCl₃. In embodiments, R³⁸ is independently —CBr₃. In embodiments, R³⁸ is independently —CI₃. In embodiments, R³⁸ is independently —F. In embodiments, R³⁸ is independently —Cl. In embodiments, R³⁸ is independently —Br. In embodiments, R³⁸ is independently —I. In embodiments, R³⁸ is independently R³⁹-substituted C₁-C₄ alkyl. In embodiments, R³⁸ is independently R³⁹-substituted 2 to 4 membered heteroalkyl. In embodiments, R³⁸ is independently R³⁹-substituted C₃-C₆ cycloalkyl. In embodiments, R³⁸ is independently R³⁹-substituted 3 to 6 membered heterocycloalkyl. In embodiments, R³⁸ is independently R³⁹-substituted phenyl. In embodiments, R³⁸ is independently R³⁹-substituted 5 to 6 membered heteroaryl. In embodiments, R³⁸ is independently unsubstituted C₁-C₄ alkyl. In embodiments, R³⁸ is independently unsubstituted 2 to 4 membered heteroalkyl. In embodiments, R³⁸ is independently unsubstituted C₃-C₆ cycloalkyl. In embodiments, R³⁸ is independently unsubstituted 3 to 6 membered heterocycloalkyl. In embodiments, R³⁸ is independently unsubstituted phenyl. In embodiments, R³⁸ is independently unsubstituted 5 to 6 membered heteroaryl.

R³⁹ is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴⁰-substituted or unsubstituted alkyl, R⁴⁰-substituted or unsubstituted heteroalkyl, R⁴⁰-substituted or unsubstituted cycloalkyl, R⁴⁰-substituted or unsubstituted heterocycloalkyl, R⁴⁰-substituted or unsubstituted aryl, or R⁴⁰-substituted or unsubstituted heteroaryl.

In embodiments, R³⁹ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R⁴⁰-substituted or unsubstituted alkyl (e.g., C₁-C₈, C₁-C₆, C₁-C₄, or C₁-C₂), R⁴⁰-substituted or unsubstituted heteroalkyl (e.g., 2 to 8 membered, 2 to 6 membered, 4 to 6 membered, 2 to 3 membered, or 4 to 5 membered), R⁴⁰-substituted or unsubstituted cycloalkyl (e.g., C₃-C₈, C₃-C₆, C₄-C₆, or C₅-C₆), R⁴⁰-substituted or unsubstituted heterocycloalkyl (e.g., 3 to 8 membered, 3 to 6 membered, 4 to 6 membered, 4 to 5 membered, or 5 to 6 membered), R⁴⁰-substituted or unsubstituted aryl (e.g., C₆-C₁₀ or phenyl), or R⁴⁰-substituted or unsubstituted heteroaryl (e.g., 5 to 10 membered, 5 to 9 membered, or 5 to 6 membered).

In embodiments, R³⁹ is independently —NH₂. In embodiments, R³⁹ is independently —OH. In embodiments, R³⁹ is independently halogen. In embodiments, R³⁹ is independently —CN. In embodiments, R³⁹ is independently oxo. In embodiments, R³⁹ is independently —CF₃. In embodiments, R³⁹ is independently —COOH. In embodiments, R³⁹ is independently —CONH₂. In embodiments, R³⁹ is independently —NO₂. In embodiments, R³⁹ is independently —SH. In embodiments, R³⁹ is independently —SO₃H. In embodiments, R³⁹ is independently —SO₄H. In embodiments, R³⁹ is independently —SO₂NH₂. In embodiments, R³⁹ is independently —NHNH₂. In embodiments, R³⁹ is independently —ONH₂. In embodiments, R³⁹ is independently —NHC═(O)NHNH₂. In embodiments, R³⁹ is independently —NHC═(O) NH₂. In embodiments, R³⁹ is independently —NHSO₂H. In embodiments, R³⁹ is independently —NHC═(O)H. In embodiments, R³⁹ is independently —NHC(O)—OH. In embodiments, R³⁹ is independently —NHOH. In embodiments, R³⁹ is independently —OCF₃. In embodiments, R³⁹ is independently —OCHF₂. In embodiments, R³⁹ is independently —CCl₃. In embodiments, R³⁹ is independently —CBr₃. In embodiments, R³⁹ is independently —CI₃. In embodiments, R³⁹ is independently —F. In embodiments, R³⁹ is independently —Cl. In embodiments, R³⁹ is independently —Br. In embodiments, R³⁹ is independently —I. In embodiments, R³⁹ is independently R⁴⁰-substituted C₁-C₄ alkyl. In embodiments, R³⁹ is independently R⁴⁰-substituted 2 to 4 membered heteroalkyl. In embodiments, R³⁹ is independently R⁴⁰-substituted C₃-C₆ cycloalkyl. In embodiments, R³⁹ is independently R⁴⁰-substituted 3 to 6 membered heterocycloalkyl. In embodiments, R³⁹ is independently R⁴⁰-substituted phenyl. In embodiments, R³⁹ is independently R⁴⁰-substituted 5 to 6 membered heteroaryl. In embodiments, R³⁹ is independently unsubstituted C₁-C₄ alkyl. In embodiments, R³⁹ is independently unsubstituted 2 to 4 membered heteroalkyl. In embodiments, R³⁹ is independently unsubstituted C₃-C₆ cycloalkyl. In embodiments, R³⁹ is independently unsubstituted 3 to 6 membered heterocycloalkyl. In embodiments, R³⁹ is independently unsubstituted phenyl. In embodiments, R³⁹ is independently unsubstituted 5 to 6 membered heteroaryl.

R²⁵, R²⁸, R³¹, R³⁴, R³⁷, and R⁴⁰ are independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, the compound is

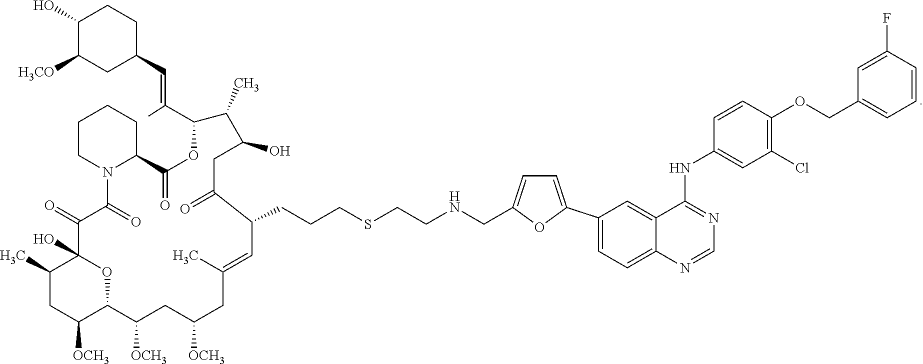 [SMILES: CO[C@H]1C/C(C)=C/[C@@H](CCCSCCNCc2ccc(-c3ccc4ncnc(Nc5ccc(OCc6cccc(F)c6)c(Cl)c5)c4c3)o2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]([C@@H](OC)C1)[C@@H](OC)C[C@H]2C]

In embodiments, the compound is

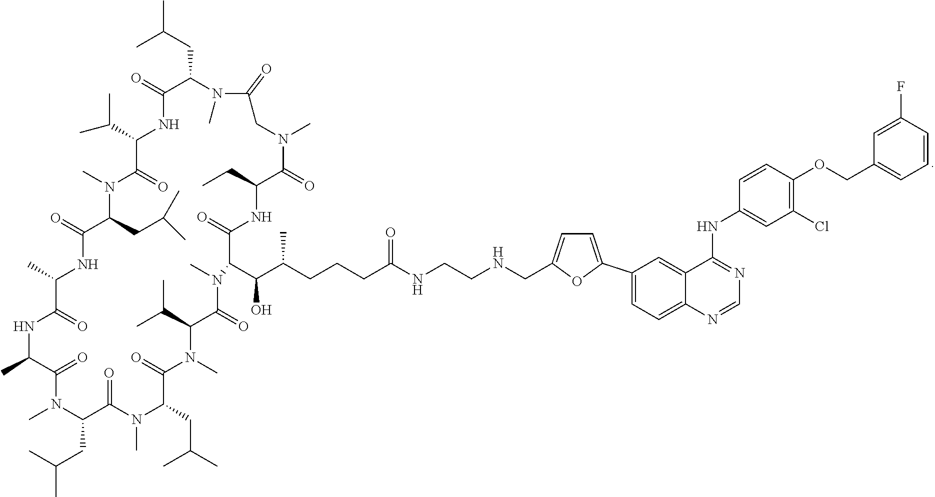 [SMILES: CC[C@@H]1NC(=O)[C@H]([C@H](O)[C@H](C)CCCC(=O)NCCNCc2ccc(-c3ccc4ncnc(Nc5ccc(OCc6cccc(F)c6)c(Cl)c5)c4c3)o2)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

In embodiments, the compound is

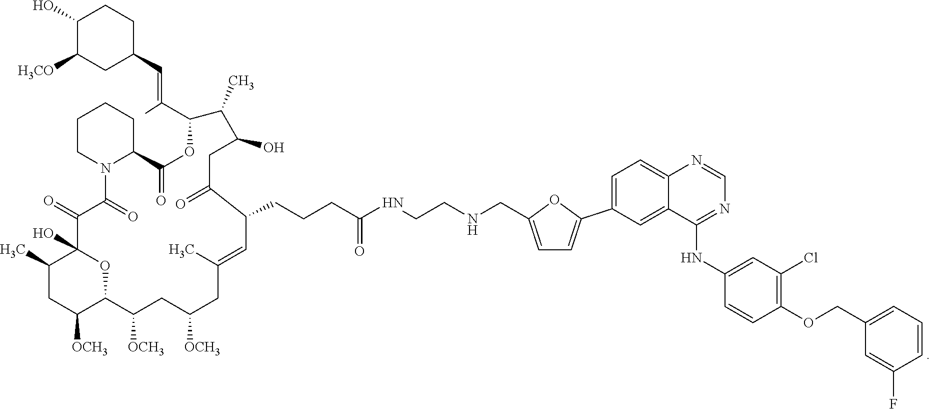 [SMILES: CO[C@H]1C/C(C)=C/[C@@H](CCCC(=O)NCCNCc2ccc(-c3ccc4ncnc(Nc5ccc(OCc6cccc(F)c6)c(Cl)c5)c4c3)o2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]([C@@H](OC)C1)[C@@H](OC)C[C@H]2C]

In embodiments, the compound is

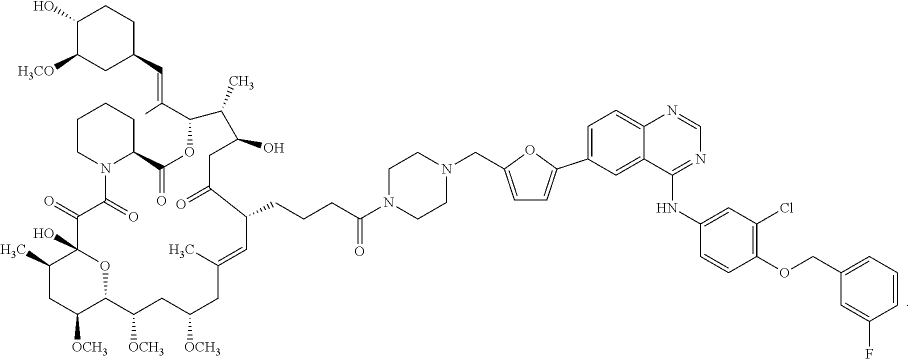 [SMILES: CO[C@H]1C/C(C)=C/[C@@H](CCCC(=O)N2CCN(Cc3ccc(-c4ccc5ncnc(Nc6ccc(OCc7cccc(F)c7)c(Cl)c6)c5c4)o3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]([C@@H](OC)C1)[C@@H](OC)C[C@H]2C]

In embodiments, the compound is

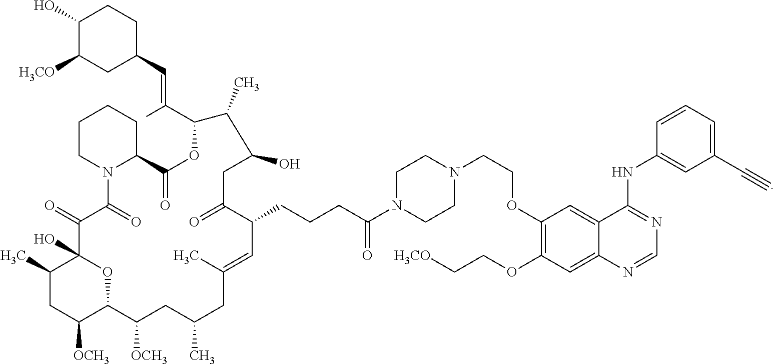

In embodiments, the compound is

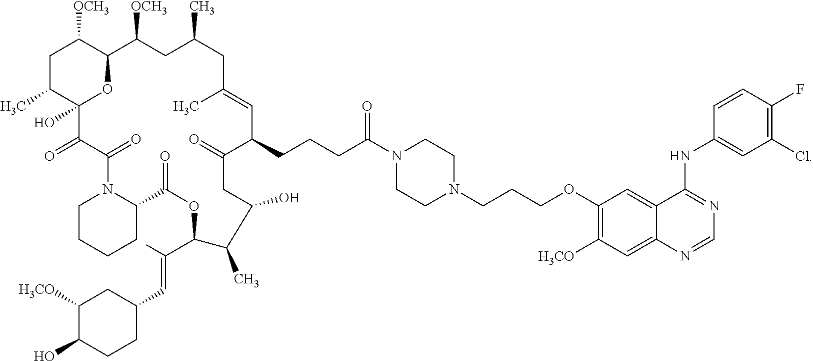 [SMILES: COc1cc2ncnc(Nc3ccc(F)c(Cl)c3)c2cc1OCCCN1CCN(C(=O)CCC[C@@H]2/C=C(\C)C[C@H](C)C[C@H](OC)[C@H]3O[C@@](O)(C(=O)C(=O)N4CCCC[C@H]4C(=O)O[C@H](/C(C)=C/[C@@H]4CC[C@@H](O)[C@H](OC)C4)[C@H](C)[C@@H](O)CC2=O)[C@H](C)C[C@@H]3OC)CC1]

In embodiments, the compound is

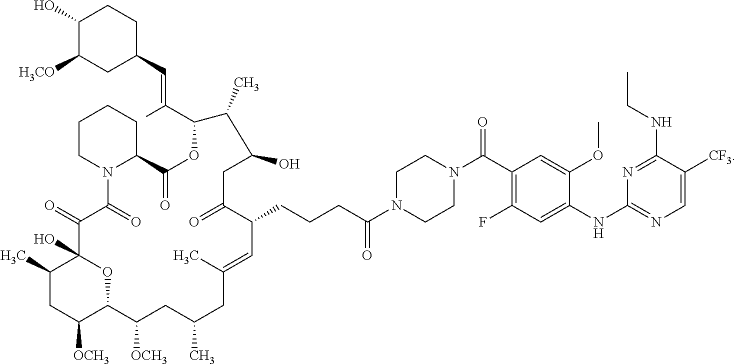 [SMILES: CCNc1nc(Nc2cc(F)c(C(=O)N3CCN(C(=O)CCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)CC3)cc2OC)ncc1C(F)(F)F]

In embodiments, the compound is

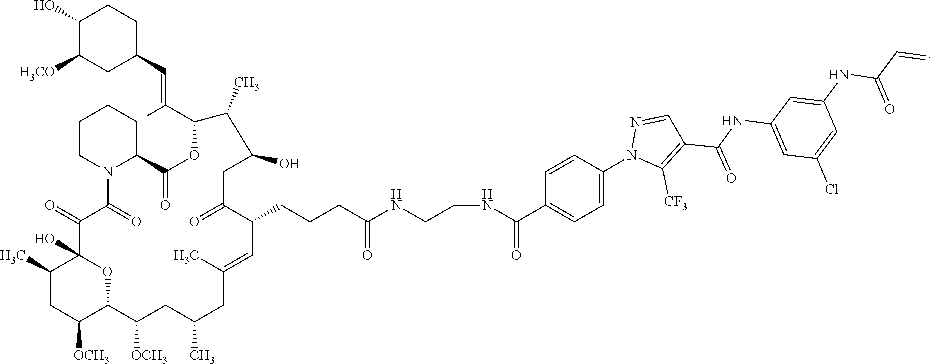

In embodiments, the compound is

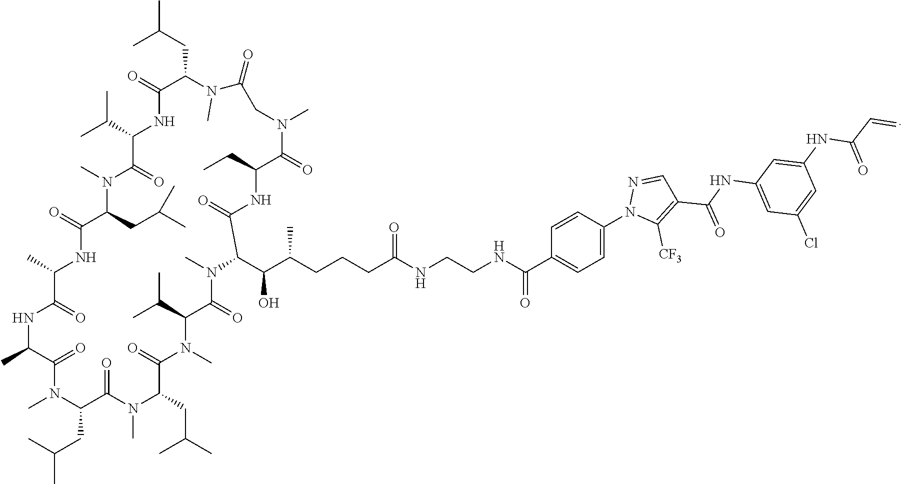

In embodiments, the compound is

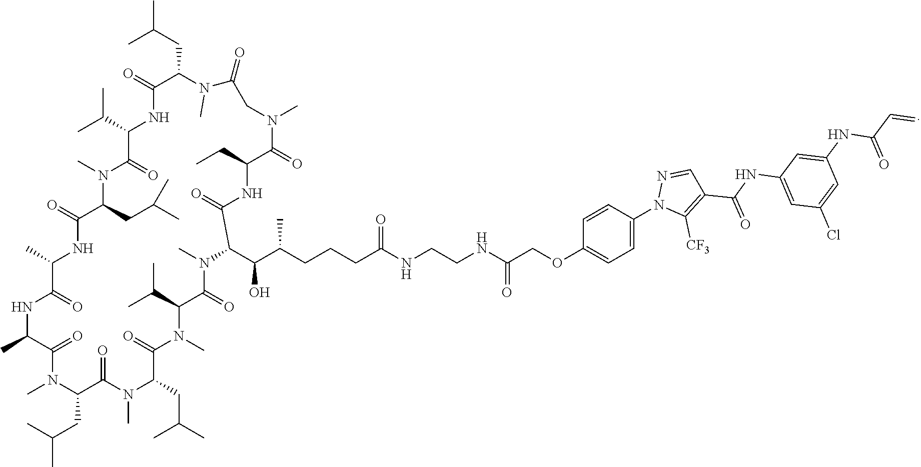

In embodiments, the compound is

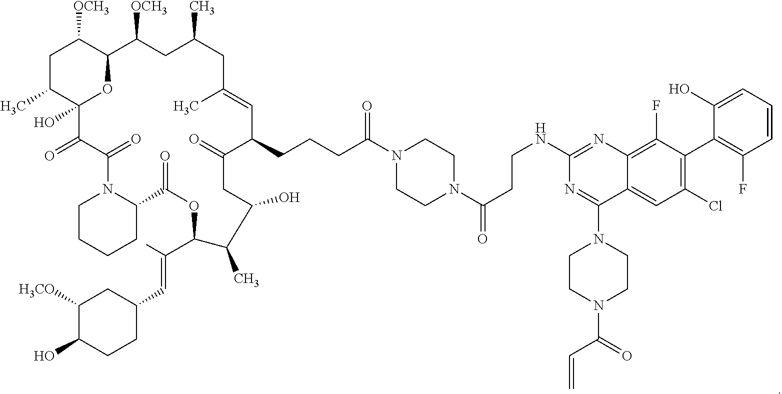 [SMILES: C=CC(=O)N1CCN(c2nc(NCCC(=O)N3CCN(C(=O)CCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)CC3)nc3c(F)c(-c4c(O)cccc4F)c(Cl)cc23)CC1 ; "."]

In embodiments, the compound is

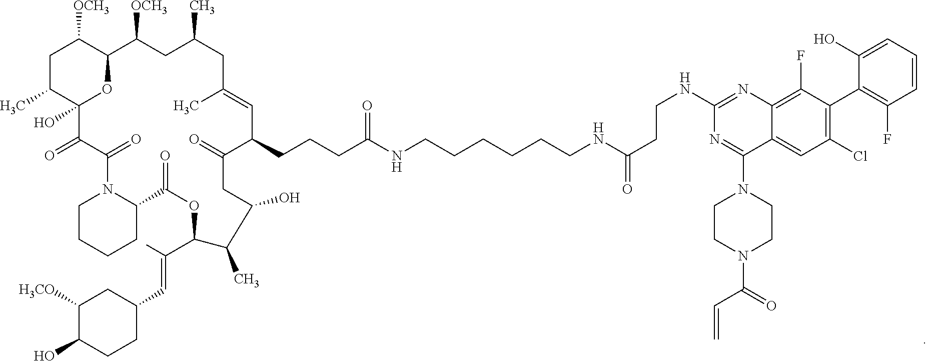 [SMILES: C=CC(=O)N1CCN(c2nc(NCCC(=O)NCCCCCCNC(=O)CCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/[C@@H]5CC[C@@H](O)[C@H](OC)C5)[C@H](C)[C@@H](O)CC3=O)[C@H](C)C[C@@H]4OC)nc3c(F)c(-c4c(O)cccc4F)c(Cl)cc23)CC1 ; "."]

In embodiments, the compound is

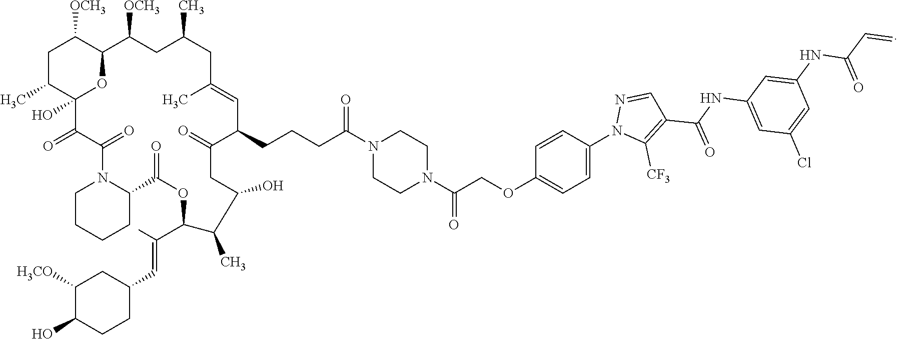

In embodiments, the compound is

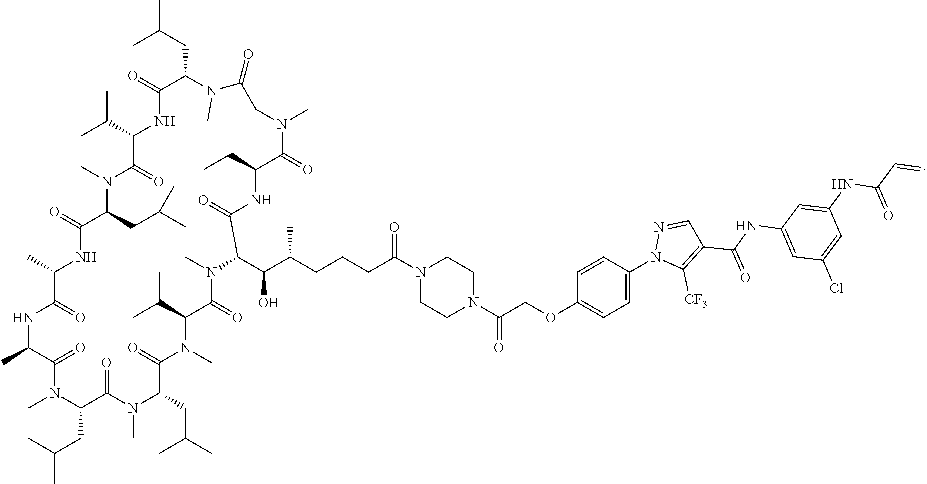

In embodiments, the compound is

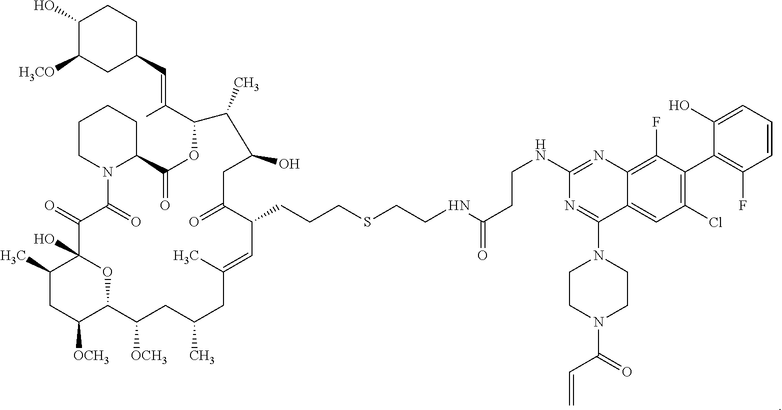 [SMILES: C=CC(=O)N1CCN(c2nc(NCCC(=O)NCCSCCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/[C@@H]5CC[C@@H](O)[C@H](OC)C5)[C@H](C)[C@@H](O)CC3=O)[C@H](C)C[C@@H]4OC)nc3c(F)c(-c4c(O)cccc4F)c(Cl)cc23)CC1 ; "."]

In embodiments, the compound is

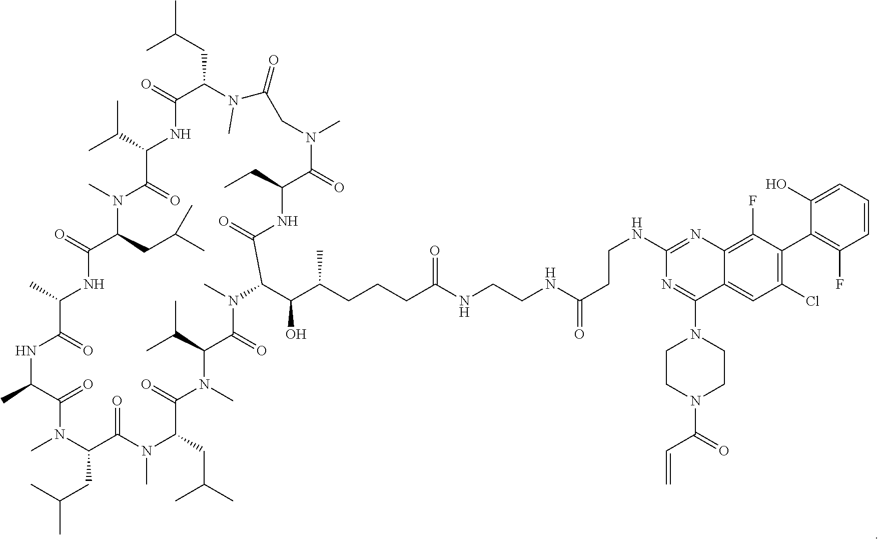 [SMILES: C=CC(=O)N1CCN(c2nc(NCCC(=O)NCCNC(=O)CCC[C@@H](C)[C@@H](O)[C@H]3C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N3C)nc3c(F)c(-c4c(O)cccc4F)c(Cl)cc23)CC1 ; "."]

In embodiments, the compound is

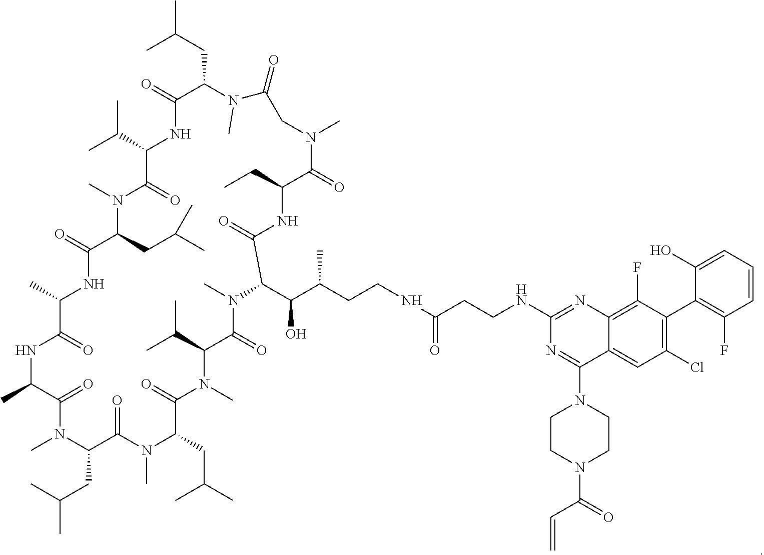 [SMILES: C=CC(=O)N1CCN(c2nc(NCCC(=O)NCC[C@@H](C)[C@@H](O)[C@H]3C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N3C)nc3c(F)c(-c4c(O)cccc4F)c(Cl)cc23)CC1 ; "."]

In embodiments, the compound is

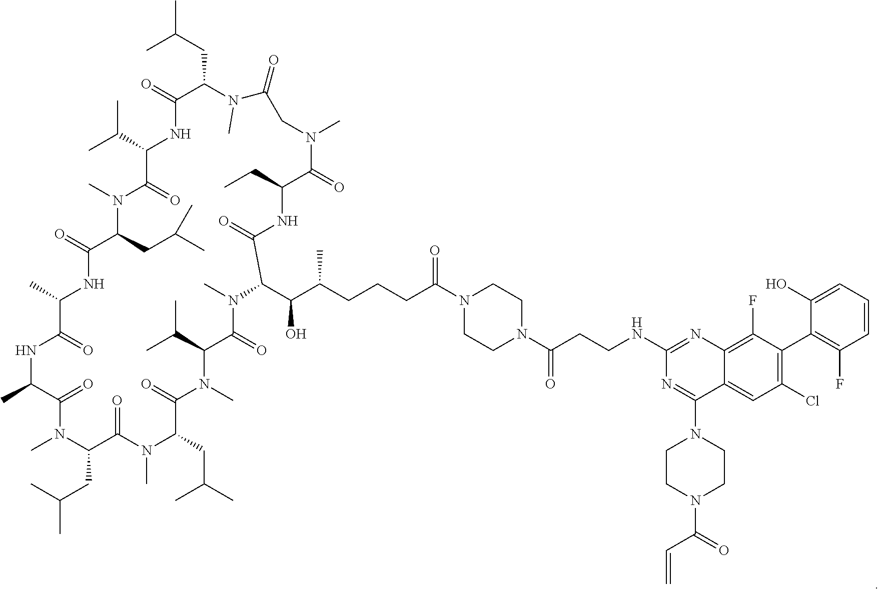 [SMILES: C=CC(=O)N1CCN(c2nc(NCCC(=O)N3CCN(C(=O)CCC[C@@H](C)[C@@H](O)[C@H]4C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N4C)CC3)nc3c(F)c(-c4c(O)cccc4F)c(Cl)cc23)CC1 ; "."]

In embodiments, the compound is

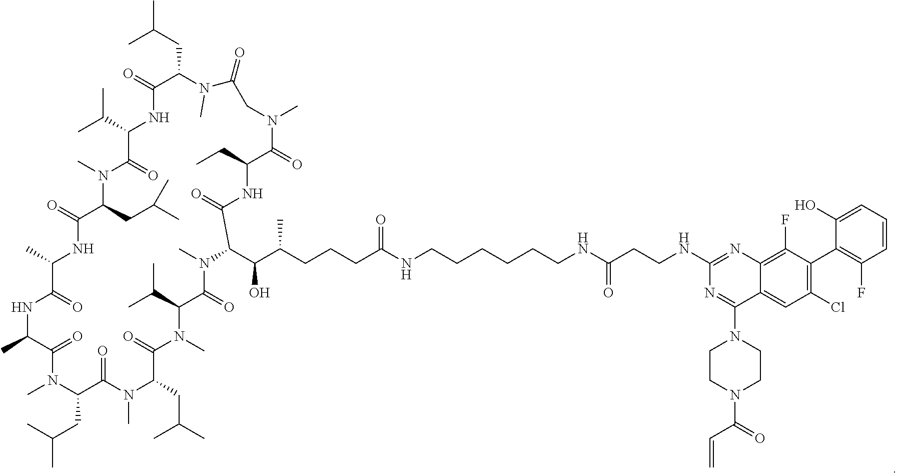 [SMILES: C=CC(=O)N1CCN(c2nc(NCCC(=O)NCCCCCCNC(=O)CCC[C@@H](C)[C@@H](O)[C@H]3C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N3C)nc3c(F)c(-c4c(O)cccc4F)c(Cl)cc23)CC1 ; "."]

In embodiments, the compound is

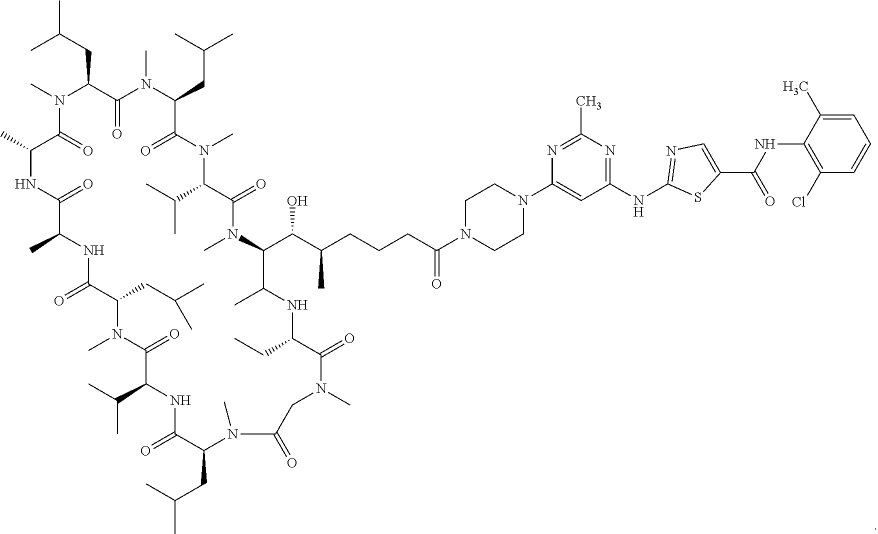 [SMILES: CC[C@@H]1NC(C)[C@H]([C@H](O)[C@H](C)CCCC(=O)N2CCN(c3cc(Nc4ncc(C(=O)Nc5c(C)cccc5Cl)s4)nc(C)n3)CC2)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

In embodiments, the compound is

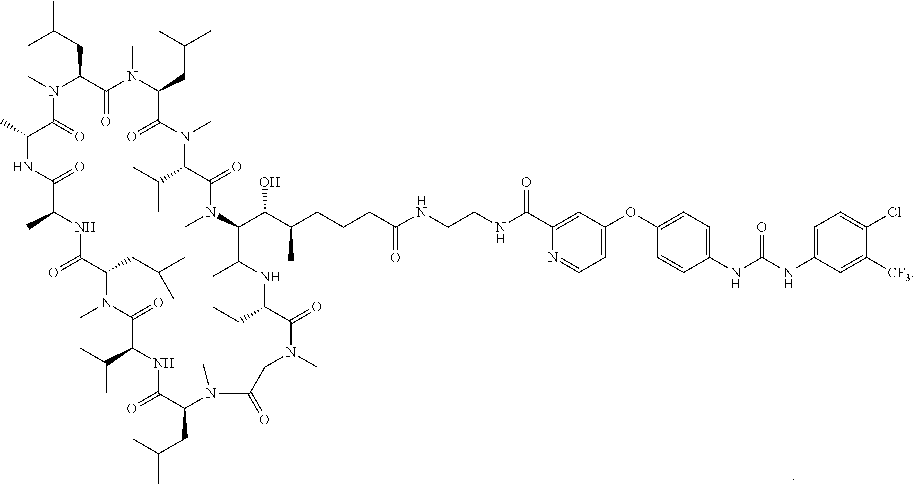 [SMILES: CC[C@@H]1NC(C)[C@H]([C@H](O)[C@H](C)CCCC(=O)NCCNC(=O)c2cc(Oc3ccc(NC(=O)Nc4ccc(Cl)c(C(F)(F)F)c4)cc3)ccn2)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

In embodiments, the compound is

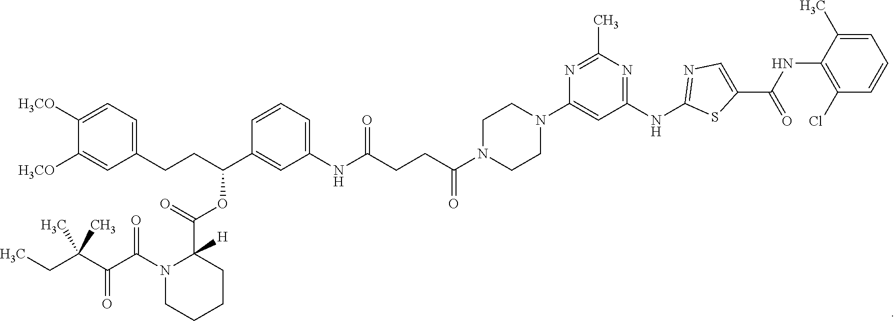

In embodiments, the compound is

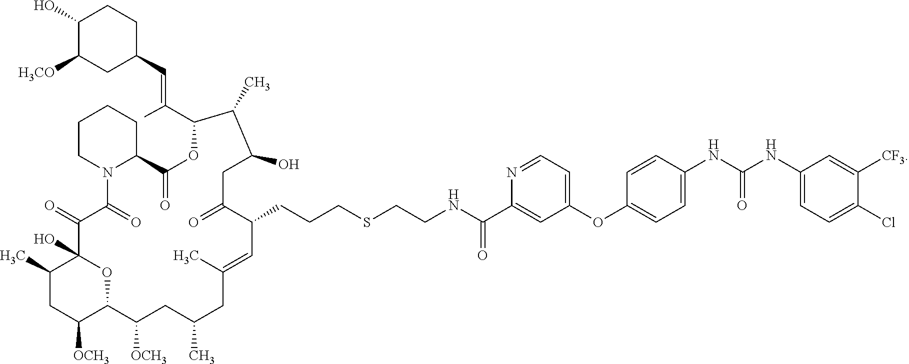 [SMILES: CO[C@H]1C[C@@H](C)C/C(C)=C/[C@@H](CCCSCCNC(=O)c2cc(Oc3ccc(NC(=O)Nc4ccc(Cl)c(C(F)(F)F)c4)cc3)ccn2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

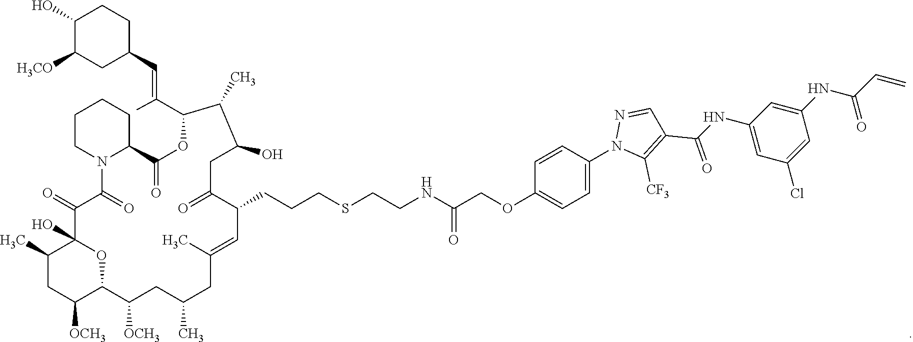 [SMILES: C=CC(=O)Nc1cc(Cl)cc(NC(=O)c2cnn(-c3ccc(OCC(=O)NCCSCCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)cc3)c2C(F)(F)F)c1]

In embodiments, the compound is

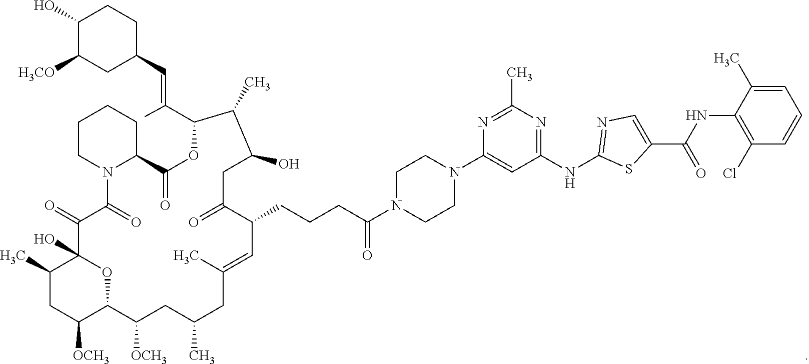 [SMILES: CO[C@H]1C[C@@H](C)C/C(C)=C/[C@@H](CCCC(=O)N2CCN(c3cc(Nc4ncc(C(=O)Nc5c(C)cccc5Cl)s4)nc(C)n3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

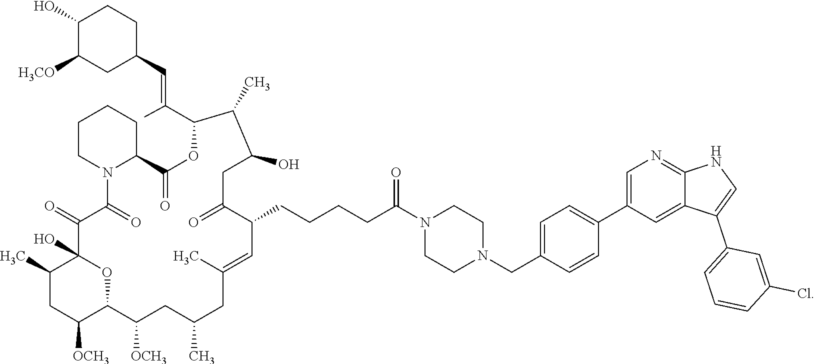 [SMILES: CO[C@H]1C[C@@H](C)C/C(C)=C/[C@@H](CCCCC(=O)N2CCN(Cc3ccc(-c4cnc5[nH]cc(-c6cccc(Cl)c6)c5c4)cc3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

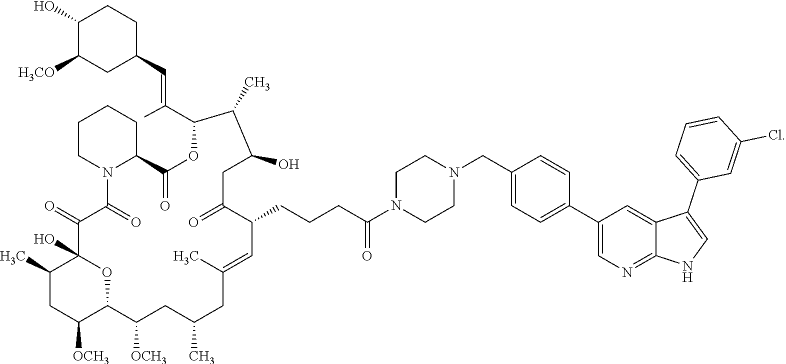 [SMILES: CO[C@H]1C[C@@H](C)C/C(C)=C/[C@@H](CCCC(=O)N2CCN(Cc3ccc(-c4cnc5[nH]cc(-c6cccc(Cl)c6)c5c4)cc3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

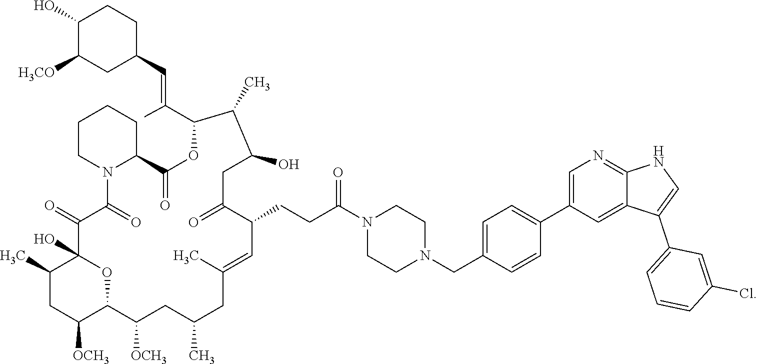 [SMILES: CO[C@H]1C[C@@H](C)C/C(C)=C/[C@@H](CCC(=O)N2CCN(Cc3ccc(-c4cnc5[nH]cc(-c6cccc(Cl)c6)c5c4)cc3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

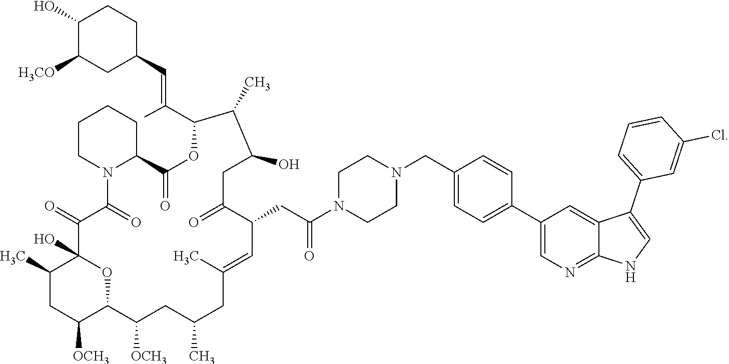 [SMILES: CO[C@H]1C[C@@H](C)C/C(C)=C/[C@@H](CC(=O)N2CCN(Cc3ccc(-c4cnc5[nH]cc(-c6cccc(Cl)c6)c5c4)cc3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

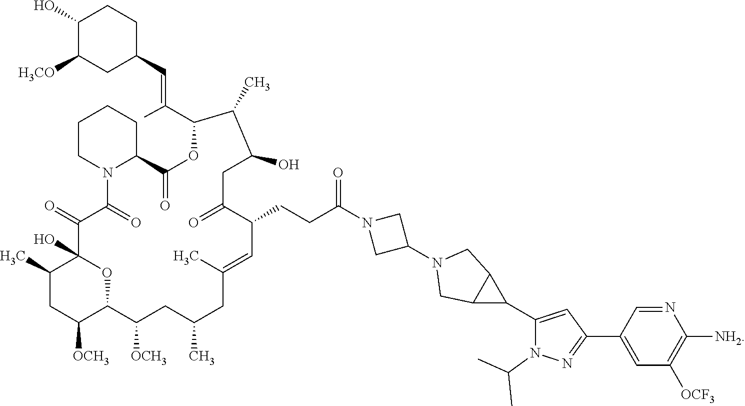 [SMILES: CO[C@H]1C[C@@H](C)C/C(C)=C/[C@@H](CCC(=O)N2CC(N3CC4C(C3)C4c3cc(-c4cnc(N)c(OC(F)(F)F)c4)nn3C(C)C)C2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

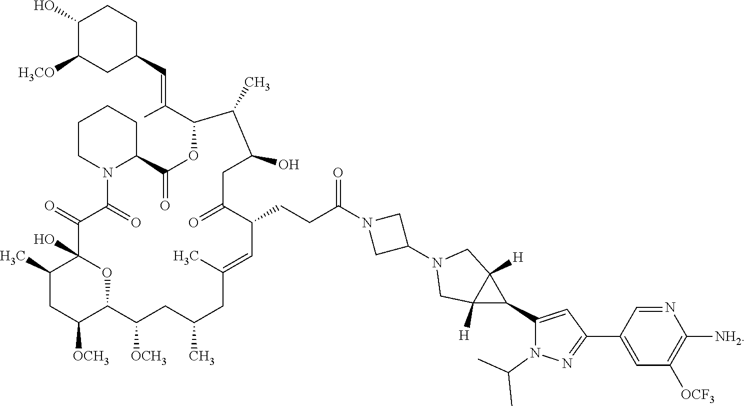 [SMILES: CO[C@H]1C[C@@H](C)C/C(C)=C/[C@@H](CCC(=O)N2CC(N3C[C@@H]4[C@H](C3)[C@H]4c3cc(-c4cnc(N)c(OC(F)(F)F)c4)nn3C(C)C)C2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

In embodiments, the compound is

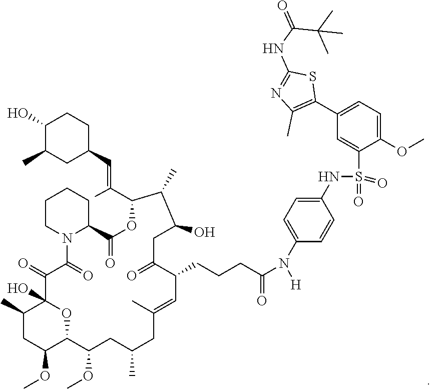

In embodiments, the compound is not

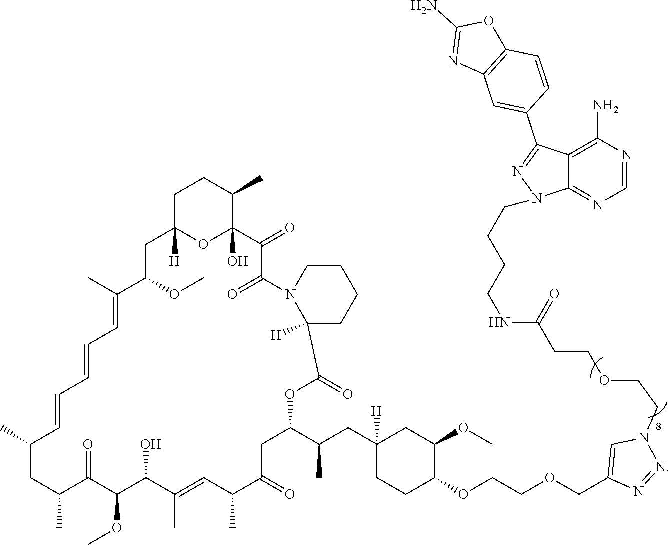

In embodiments, the compound does not include a monovalent active site mTOR inhibitor covalently bound to a monovalent rapamycin or a monovalent rapamycin analog.

In embodiments, compound does not include a divalent linker that binds the monovalent active site mTOR inhibitor (active site mTOR inhibitor moiety) to the monovalent rapamycin (rapamycin moiety) or the monovalent rapamycin analog (rapamycin analog moiety). In embodiments, the divalent linker is not bonded to rapamycin or a rapamycin analog at a position capable of being modified to include a linker. For example, a linker may not be bonded to rapamycin or a rapamycin analog at position 10, 16, 27, 28, 39, or 40, among others (as indicated in figure immediately below). In embodiments, a linker is not bonded to position 10 of rapamycin or a rapamycin analog. In embodiments, a linker is not bonded to position 16 of rapamycin or a rapamycin analog. In embodiments, a linker is not bonded to position 27 of rapamycin or a rapamycin analog. In embodiments, a linker is not bonded to position 28 of rapamycin or a rapamycin analog. In embodiments, a linker is not bonded to position 39 of rapamycin or a rapamycin analog. In embodiments, a linker is not bonded to position 40 of rapamycin or a rapamycin analog.

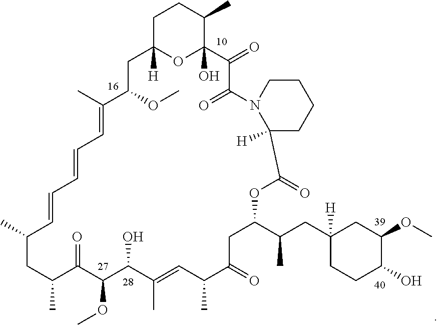

In embodiments, the divalent linker is not at least or about 5 Ain length (e.g., at least or about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Ain length). In embodiments, the divalent linker is not at least or about the length of 5 methylene groups (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 methylene groups). In embodiments, the divalent linker is not at least or about the length of 11 methylene groups (e.g., at least or about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 methylene groups). In embodiments, the divalent linker is not at least or about the length of 27 methylene groups (e.g., 27, 28, 29, 30, 31, 32, 33, 34, 35, 36, 37, 38, 39, 40, 41, 42, 43, 44, 45, 46, 47, 48, 49, or 50 methylene groups).

It will be understood that a linker may adopt a through space distance (e.g., in solution, when bound to mTORC1, when bound to mTOR) that is less than the fully extended conformation used to define the linker length.

In embodiments, the linker is not a hydrolysable linker (e.g., in solution). In embodiments, the linker is not a non-hydrolysable linker (e.g., in solution). In embodiments, the linker is not cleaved by an enzyme (e.g., hydrolase, protease, cytochrome). In embodiments, the linker is cleavable by an enzyme (e.g., under normal cellular conditions). In embodiments, the linker is not a polyethylene glycol linker. In embodiments, the linker is not hydrophilic. In embodiments, the linker is not hydrophobic. In embodiments, the linker does not include a disulfide bond. In embodiments, the linker does not include a hydrazone bond. In embodiments, the linker does not include an ester. In embodiments, the linker does not include a sulfonyl. In embodiments, the linker does not include a thioether. In embodiments, the linker does not include a phosphinate. In embodiments, the linker does not include an alkyloxime bond. In embodiments, the linker does not include one or more amino acids.

In embodiments, the compound does not include a divalent linker covalently bound to the monovalent active site mTOR inhibitor and the monovalent rapamycin or monovalent rapamycin analog. In embodiments, the compound does not include a divalent linker covalently bound directly to the monovalent active site mTOR inhibitor and directly to the monovalent rapamycin or monovalent rapamycin analog.

In embodiments, the compound does not have the formula:

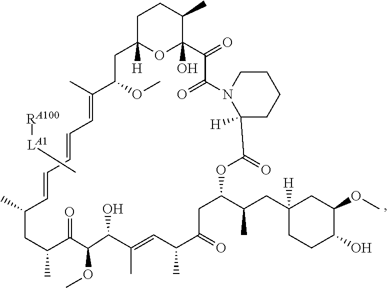

wherein L^{A1} is as described herein and may be bonded to any atom in the ring (L^{A1} is a floating substituent) and R^{A100} is a monovalent active site mTOR inhibitor.

In embodiments, the compound does not have the formula:

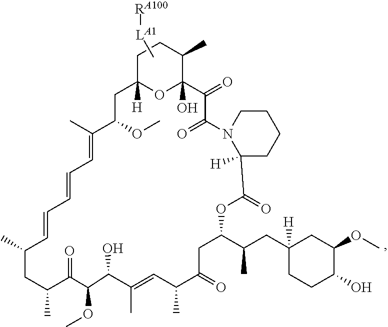

wherein L^{A1} is as described herein and may be bonded to any atom in the ring (L^{A1} is a floating substituent) and R^{A100} is a monovalent active site mTOR inhibitor.

In embodiments, the compound does not have the formula:

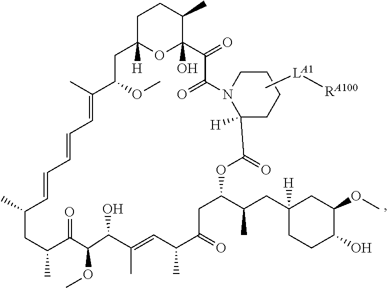

wherein L^{A1} is as described herein and may be bonded to any atom in the ring (L^{A1} is a floating substituent) and R^{A100} is a monovalent active site mTOR inhibitor.

In embodiments, the compound does not have the formula:

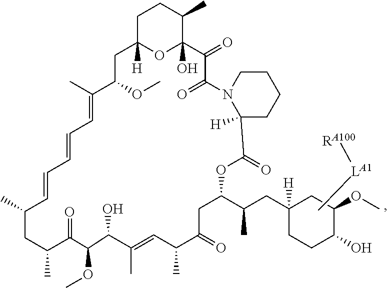

wherein L^{A1} is as described herein and may be bonded to any atom in the ring (L^{A1} is a floating substituent) and R^{A100} is a monovalent active site mTOR inhibitor.

R^{A100} is a monovalent active site mTOR inhibitor. In embodiments, R^{A100} is

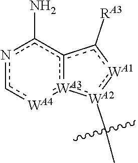

wherein W^{A1}, W^{A2}, W^{A3}, W^{A4}, and R^{A3} are as described herein. In embodiments, R^{A100} is

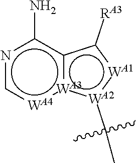

wherein W^{A1}, W^{A2}, W^{A3}, W^{A4}, and R^{A3} are as described herein. In embodiments, R^{A100} is

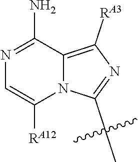

wherein R^{A3} and R^{A12} are as described herein. In embodiments, R^{A100} is

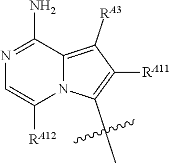

wherein R^{A3}, R^{A11}, and R^{A12} are as described herein. In embodiments, R^{A100} is

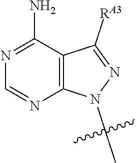

wherein R^{A3} is as described herein. In embodiments, R^{A100} is

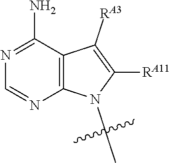

wherein R^{A3} and R^{A11} are as described herein. In embodiments, R^{A100} is

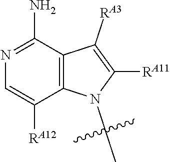

wherein R^{A3}, R^{A11}, and R^{A12} are as described herein. In embodiments, R^{A100} is

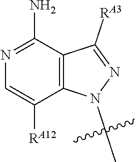

wherein R^{A3} and R^{A12} are as described herein.

In embodiments, the compound does not have the formula:

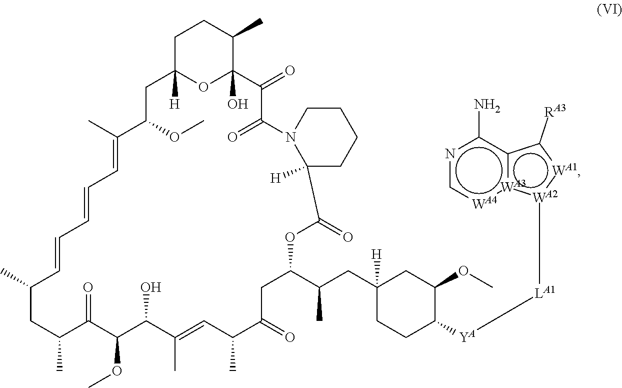

wherein W^{A1}, W^{A2}, W^{A3}, W^{A4}, L^{A1}, Y^{A}, and R^{A3} are as described herein.

In embodiments, the compound does not have the formula:

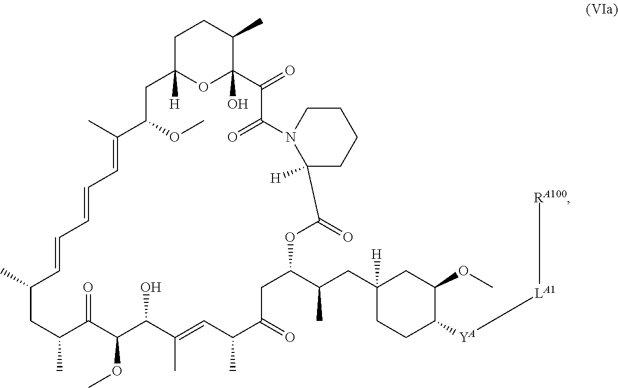

wherein L^{A1}, Y^{A}, and R^{A100} are as described herein.

In embodiments, the compound does not have the formula:

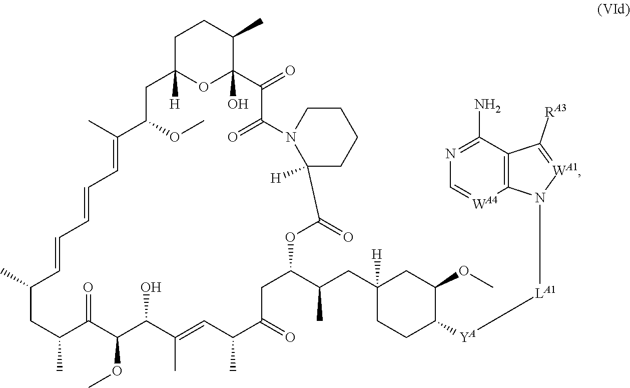

R^{A3}, W^{A1}, and W^{A4} are as described herein.

L^{A1} is a covalent linker as described herein. W^{A1} is N or CR^{A11}. W^{A2} is N and W^{A3} is C or, alternatively, W^{A2} is C and W^{A3} is N. W^{A4} is N or CR^{A12}. Y^{A} is O or NR^{A13}, R^{A3} is hydrogen, oxo, halogen, —CX^{A}₃, —CN, —SO₂Cl, —SO_{nA}R^{A10}, —SO_{VA}NR^{A7}R^{A8}, —NHNH₂—ONR^{A7}R^{A8}, —NHC(O)NHNH₂,

—NHC(O)NR^{A7}R^{A8}, —N(O)_{mA}, —NR^{A7}R^{A8}, —C(O)R^{A9}, —C(O)OR^{A9}, —C(O)NR^{A7}R^{A8}, —OR^{A10}, —NR^{A7}SO₂R^{A10}, —NR^{A7}C(O)R^{A9}, —NR^{A7}C(O)OR^{A9}, —NR^{A7}OR^{A9}, —OCX^{A}₃, —OCHX^{A}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{A7}, R^{A8}, R^{A9}, R^{A10}, R^{A11}, R^{A12} and R^{A13} are independently hydrogen, halogen, —CF₃, —CN, —OH,
—NH₂, —COOH, —CONH₂, —NO₂, —SH, —SO₃H, —SO₄H, —SO₂NH₂, —NHNH₂, —ONH₂, —NHC(O)NHNH₂,
—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{A7} and R^{A8} substituents bonded to the same nitrogen atom may optionally be joined to form a substituted or unsubstituted heterocycloalkyl or substituted or unsubstituted heteroaryl. The variables mA and vA are independently 1 or 2. The variable nA is independently an integer from 0 to 4. The variable X^{A} is independently —Cl, —Br, —I, or —F.

In embodiments, R^{A3} is hydrogen, oxo, halogen, —CX^{A3}, —CN, —SO₂Cl, —SO_{nA}R^{A10}, —SO_{VA}NR^{A7}R^{A8}, —NHNH₂, —ONR^{A7}R^{A8}, —NHC(O)NHNH₂, —NHC(O)NR^{A7}R^{A8}, —N(O)_{mA}, —NR^{A7}R^{A8}, —C(O)R^{A9}, —C(O)OR^{A9}, —C(O)NR^{A7}R^{A8}, —OR^{A10}, —NR^{A7}SO₂R^{A10}, —NR^{A7}C(O)R^{A9}, —NR^{A7}C(O)OR^{A9}, —NR^{A7}OR^{A9}, —OCX^{A3}, —OCHX^{A2}, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A3} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A3} is substituted with a plurality of groups selected from substituent groups, size-limited substituent groups, and lower substituent groups; each substituent group, size-limited substituent group, and/or lower substituent group may optionally be different. In embodiments, when R^{A3} is substituted, it is substituted with at least one substituent group. In embodiments, when R^{A3} is substituted, it is substituted with at least one size-limited substituent group. In embodiments, when R^{A3} is substituted, it is substituted with at least one lower substituent group.

In embodiments, R^{A7} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{A7} and R^{A8} substituents bonded to the same nitrogen atom may optionally be joined to form a substituted or unsubstituted heterocycloalkyl or substituted or unsubstituted heteroaryl.

In embodiments, R^{A7} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A7} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A7} is substituted with a plurality of groups selected from substituent groups, size-limited substituent groups, and lower substituent groups; each substituent group, size-limited substituent group, and/or lower substituent group may optionally be different. In embodiments, when R^{A7} is substituted, it is substituted with at least one substituent group. In embodiments, when R^{A7} is substituted, it is substituted with at least one size-limited substituent group. In embodiments, when R^{A7} is substituted, it is substituted with at least one lower substituent group.

In embodiments, R^{A8} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{A8} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A8} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A8} is substituted with a plurality of groups selected from substituent groups, size-limited substituent groups, and lower substituent groups; each substituent group, size-limited substituent group, and/or lower substituent group may optionally be different. In embodiments, when R^{A8} is substituted, it is substituted with at least one substituent group. In embodiments, when R^{A8} is substituted, it is substituted with at least one size-limited substituent group. In embodiments, when R^{A8} is substituted, it is substituted with at least one lower substituent group.

In embodiments, R^{A7} and R^{A8} substituents bonded to the same nitrogen atom may optionally be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered) or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted moiety formed by joining R^{A7} and R^{A8} substituents bonded to the same nitrogen atom (e.g., substituted heterocycloalkyl and/or substituted heteroaryl) is substituted with at least one substituent group, size-limited substituent group, or lower substituent group; wherein if the substituted moiety formed by joining R^{A7} and R^{A8} substituents bonded to the same nitrogen atom is substituted with a plurality of groups selected from substituent groups, size-limited substituent groups, and lower substituent groups; each substituent group, size-limited substituent group, and/or lower substituent group may optionally be different. In embodiments, when the moiety formed by joining R^{A7} and R^{A8} substituents bonded to the same nitrogen atom is substituted, it is substituted with at least one substituent group. In embodiments, when the moiety formed by joining R^{A7} and R^{A8} substituents bonded to the same nitrogen atom is substituted, it is substituted with at least one size-limited substituent group. In embodiments, when the moiety formed by joining R^{A7} and R^{A8} substituents bonded to the same nitrogen atom is substituted, it is substituted with at least one lower substituent group.

In embodiments, R^{A9} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{A9} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A9} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A9} is substituted with a plurality of groups selected from substituent groups, size-limited substituent groups, and lower substituent groups; each substituent group, size-limited substituent group, and/or lower substituent group may optionally be different. In embodiments, when R^{A9} is substituted, it is substituted with at least one substituent group. In embodiments, when R^{A9} is substituted, it is substituted with at least one size-limited substituent group. In embodiments, when R^{A9} is substituted, it is substituted with at least one lower substituent group.

In embodiments, R^{A10} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{A10} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A10} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A10} is substituted with a plurality of groups selected from substituent groups, size-limited substituent groups, and lower substituent groups; each substituent group, size-limited substituent group, and/or lower substituent group may optionally be different. In embodiments, when R^{A10} is substituted, it is substituted with at least one substituent group. In embodiments, when R^{A10} is substituted, it is substituted with at least one size-limited substituent group. In embodiments, when R^{A10} is substituted, it is substituted with at least one lower substituent group.

In embodiments, R^{A11} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{A11} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A11} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A11} is substituted with a plurality of groups selected from substituent groups, size-limited substituent groups, and lower substituent groups; each substituent group, size-limited substituent group, and/or lower substituent group may optionally be different. In embodiments, when R^{A11} is substituted, it is substituted with at least one substituent group. In embodiments, when R^{A11} is substituted, it is substituted with at least one size-limited substituent group. In embodiments, when R^{A11} is substituted, it is substituted with at least one lower substituent group.

In embodiments, R^{A12} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{A12} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A12} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A12} is substituted with a plurality of groups selected from substituent groups, size-limited substituent groups, and lower substituent groups; each substituent group, size-limited substituent group, and/or lower substituent group may optionally be different. In embodiments, when R^{A12} is substituted, it is substituted with at least one substituent group. In embodiments, when R^{A12} is substituted, it is substituted with at least one size-limited substituent group. In embodiments, when R^{A12} is substituted, it is substituted with at least one lower substituent group.

In embodiments, R^{A13} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂,

—NHC(O)NH₂, —NHSO₂H, —NHC(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{A13} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R^{A13} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A13} is substituted with a plurality of groups selected from substituent groups, size-limited substituent groups, and lower substituent groups; each substituent group, size-limited substituent group, and/or lower substituent group may optionally be different. In embodiments, when R^{A13} is substituted, it is substituted with at least one substituent group. In embodiments, when R^{A13} is substituted, it is substituted with at least one size-limited substituent group. In embodiments, when R^{A13} is substituted, it is substituted with at least one lower substituent group.

In embodiments, the compound does not have the formula:

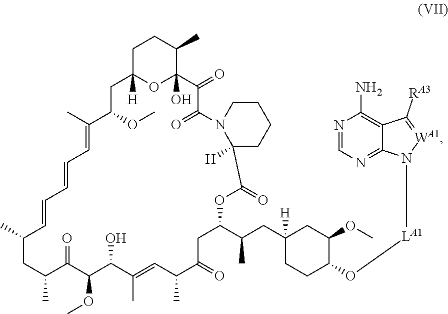

wherein W^{A1} is N or CH. In embodiments, W^{A1} is not N. In embodiments, W^{A1} is not CH.

In embodiments, the compound does not have the formula:

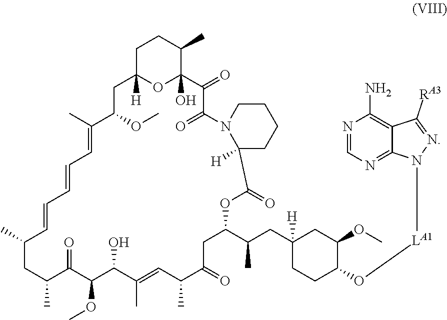

In embodiments, R^{A3} is not independently substituted benzoxazolyl, substituted pyrimidinyl, substituted thiophenyl, substituted furanyl, substituted indolyl, substituted benzoxadiazolyl, substituted benzodioxolyl, substituted benzodioxanyl, substituted thianaphthanyl, substituted pyrrolopyridinyl, substituted indazolyl, substituted quinolinyl, substituted quinoxalinyl, substituted pyridopyrazinyl, substituted quinazolinonyl, substituted benzoisoxazolyl, substituted imidazopyridinyl, substituted benzofuranyl, substituted benzothiophenyl, substituted phenyl, substituted naphthyl, substituted biphenyl, substituted pyrrolyl, substituted pyrazolyl, substituted imidazolyl, substituted pyrazinyl, substituted oxazolyl, substituted isoxazolyl, substituted thiazolyl, substituted furylthienyl, substituted pyridyl, substituted pyrimidyl, substituted benzothiazolyl, substituted purinyl, substituted benzimidazolyl, substituted isoquinolyl, substituted thiadiazolyl, substituted oxadiazolyl, substituted pyrrolyl, substituted diazolyl, substituted triazolyl, substituted tetrazolyl, substituted benzothiadiazolyl, substituted isothiazolyl, substituted pyrazolopyrimidinyl, substituted pyrrolopyrimidinyl, substituted benzotriazolyl, or substituted quinolyl. In embodiments, R^{A3} is not independently substituted benzoxazolyl.

In embodiments, R^{A3} is not independently hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R^{A20}-substituted or unsubstituted alkyl, R^{A20}-substituted or unsubstituted heteroalkyl, R^{A20}-substituted or unsubstituted cycloalkyl, R^{A20}-substituted or unsubstituted heterocycloalkyl, R^{A20}-substituted or unsubstituted aryl, or R^{A20}-substituted or unsubstituted heteroaryl.

In some embodiments, R^{A3} is not substituted with one or more substituents independently selected from halogen, —CF₃, —OH, and —NH₂. In some embodiments, R^{A3} is not substituted heteroaryl, such as benzoxazolyl or benzothiazolyl. In some embodiments, R^{A3} is not heteroaryl, such as benzoxazolyl or benzothiazolyl, substituted with one or more substituents independently selected from halogen, —CF₃, —OH, and —NH₂.

In embodiments, R^{A3} is not independently R^{A20}-substituted benzoxazolyl, R^{A20}-substituted pyrimidinyl, R^{A20}-substituted thiophenyl, R^{A20}-substituted furanyl, R^{A20}-substituted indolyl, R^{A20}-substituted benzoxadiazolyl, R^{A20}-substituted benzodioxolyl, R^{A20}-substituted benzodioxanyl, R^{A20}-substituted thianaphthanyl, R^{A20}-substituted pyrrolopyridinyl, R^{A20}-substituted indazolyl, R^{A20}-substituted quinolinyl, R^{A20}-substituted quinoxalinyl, R^{A20}-substituted pyridopyrazinyl, R^{A20}-substituted quinazolinonyl, R^{A20}-substituted benzoisoxazolyl, R^{A20}-substituted imidazopyridinyl, R^{A20}-substituted benzofuranyl, R^{A20}-substituted benzothiophenyl, R^{A20}-substituted phenyl, R^{A20}-substituted naphthyl, R^{A20}-substituted biphenyl, R^{A20}-substituted pyrrolyl, R^{A20}-substituted pyrazolyl, R^{A20}-substituted imidazolyl, R^{A20}-substituted pyrazinyl, R^{A20}-substituted oxazolyl, R^{A20}-substituted isoxazolyl, R^{A20}-substituted thiazolyl, R^{A20}-substituted furylthienyl, R^{A20}-substituted pyridyl, R^{A20}-substituted pyrimidyl, R^{A20}-substituted benzothiazolyl, R^{A20}-substituted purinyl, R^{A20}-substituted benzimidazolyl, R^{A20}-substituted isoquinolyl, R^{A20}-substituted thiadiazolyl, R^{A20}-substituted oxadiazolyl, R^{A20}-substituted pyrrolyl, R^{A20}-substituted diazolyl, R^{A20}-substituted triazolyl, R^{A20}-substituted tetrazolyl, R^{A20}-substituted benzothiadiazolyl, R^{A20}-substituted isothiazolyl, R^{A20}-substituted pyrazolopyrimidinyl, R^{A20}-substituted pyrrolopyrimidinyl, R^{A20}-substituted benzotriazolyl, or R^{A20}-substituted quinolyl. In embodiments, R^{A3} is not independently R^{A20}-substituted benzoxazolyl.

R^{A20} is independently oxo,

halogen, —CF₃, —CN, —OH, —NH₂, —COOH, —CONH₂, —NO₂, —SH,
—SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R^{A21}-substituted or unsubstituted alkyl, R^{A21}-substituted or unsubstituted heteroalkyl, R^{A21}-substituted or unsubstituted cycloalkyl, R^{A21}-substituted or unsubstituted heterocycloalkyl, R^{A21}-substituted or unsubstituted aryl, or R^{A21}-substituted or unsubstituted heteroaryl.

In embodiments, R^{A20} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R^{A21}-substituted or unsubstituted alkyl (e.g., C₁-C₈, C₁-C₆, C₁-C₄, or C₁-C₂), R^{A21}-substituted or unsubstituted heteroalkyl (e.g., 2 to 8 membered, 2 to 6 membered, 4 to 6 membered, 2 to 3 membered, or 4 to 5 membered), R^{A21}-substituted or unsubstituted cycloalkyl (e.g., C₃-C₈, C₃-C₆, C₄-C₆, or C₅-C₆), R^{A21}-substituted or unsubstituted heterocycloalkyl (e.g., 3 to 8 membered, 3 to 6 membered, 4 to 6 membered, 4 to 5 membered, or 5 to 6 membered), R^{A21}-substituted or unsubstituted aryl (e.g., C₆-C₁₀ or phenyl), or R^{A21}-substituted or unsubstituted heteroaryl (e.g., 5 to 10 membered, 5 to 9 membered, or 5 to 6 membered).

R^{A21} is independently oxo,

halogen, —CF₃, —CN, —OH, —NH₂, —COOH, —CONH₂, —NO₂, —SH,
—SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R^{A22}-substituted or unsubstituted alkyl, R^{A22}-substituted or unsubstituted heteroalkyl, R^{A22}-substituted or unsubstituted cycloalkyl, R^{A22}-substituted or unsubstituted heterocycloalkyl, R^{A22}-substituted or unsubstituted aryl, or R^{A22}-substituted or unsubstituted heteroaryl.

In embodiments, R^{A21} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, R^{A22}-substituted or unsubstituted alkyl (e.g., C₁-C₈, C₁-C₆, C₁-C₄, or C₁-C₂), R^{A22}-substituted or unsubstituted heteroalkyl (e.g., 2 to 8 membered, 2 to 6 membered, 4 to 6 membered, 2 to 3 membered, or 4 to 5 membered), R^{A22}-substituted or unsubstituted cycloalkyl (e.g., C₃-C₈, C₃-C₆, C₄-C₆, or C₅-C₆), R^{A22}-substituted or unsubstituted heterocycloalkyl (e.g., 3 to 8 membered, 3 to 6 membered, 4 to 6 membered, 4 to 5 membered, or 5 to 6 membered), R^{A22}-substituted or unsubstituted aryl (e.g., C₆-C₁₀ or phenyl), or R^{A22}-substituted or unsubstituted heteroaryl (e.g., 5 to 10 membered, 5 to 9 membered, or 5 to 6 membered).

R^{A22} is independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

In embodiments, R^{A22} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In some embodiments, L^{A1} is not L^{A2}-L^{A3}-L^{A4}-L^{A5}; L^{A2} is —CH₂CH₂OCH₂—; L^{A3} is 5 to 10 membered heteroarylene; L^{A4} is —(CH₂CH₂O)_{eA}—; eA is an integer from 2 to 8; L^{A5} is —CH₂CH₂C(O)NH(CH₂)_{eA10}—; and eA10 is an integer from 1 to 6. In some embodiments, L^{A1} is not L^{A2}-L^{A3}-L^{A4}-L^{A5}; L^{A2} is 2 to 8 membered heteroalkylene comprising at least one NH or O; L^{A3} is 5 to 10 membered heteroarylene; L^{A4} is —[(CH₂)_{eA11}O]_{eA12}—; eA11 is an integer from 1 to 3; eA12 is an integer from 1 to 8; L^{A5} is —CH₂CH₂C(O)NH(CH₂)_{eA10}; and eA10 is an integer from 1 to 6. In some embodiments, L^{A1} is not L^{A2}-L^{A3}-L^{A4}-L^{A5}; L^{A2} is —CH₂CH₂OCH₂—; L^{A3} is 5 membered heteroarylene; L^{A4} is —(CH₂CH₂O)_{eA}—; eA is an integer from 4 to 8; and L^{A5} is —CH₂CH₂C(O)NH(CH₂)₄. In some embodiments, L^{A1} is not L^{A2}-L^{A3}-L^{A4}-L^{A5}; L^{A2} is —CH₂CH₂OCH₂—; L^{A3} is triazolylene; L^{A4} is —(CH₂CH₂O)_{eA}—; eA is an integer from 4 to 8; and L^{A5} is —CH₂CH₂C(O)NH(CH₂)₄. In some embodiments, L^{A1} is L^{A2}-L^{A3}-L^{A4}-L^{A5}; L^{A2} is —CH₂CH₂OCH₂—; L^{A3} is 5 to 10 membered heteroarylene; L^{A4} is —(CH₂)_{eA}—; eA is an integer from 2 to 8; and L^{A5} is a bond.

In embodiments, L^{A2} is not substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L^{A2} is not substituted or unsubstituted 3 to 8 membered heteroalkylene. In embodiments, L^{A2} is not —CH₂CH₂OCH₂—. In embodiments, L^{A2} is not unsubstituted 3 to 8 membered heteroalkylene. In embodiments, L^{A2} is not unsubstituted 3 to 6 membered heteroalkylene. In embodiments, L^{A2} is not unsubstituted 3 to 5 membered heteroalkylene. In embodiments, L^{A2} is not a divalent linker including one or more amino acids. In embodiments, L^{A2} is not a divalent linker consisting of amino acids. In embodiments, L^{A2} is not a divalent linker including an amino acid analog. In embodiments, L^{A2} is not a divalent linker including an amino acid mimetic. In embodiments, L^{A2} is not a divalent linker consisting of amino acid analogs. In embodiments, L^{A2} is not a divalent linker consisting of amino acid mimetics.

In embodiments, L^{A3} is not a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L^{A3} is not a substituted or unsubstituted 5 to 10 membered heteroarylene. In embodiments, L^{A3} is not a bond. In embodiments, L^{A3} is not a substituted or unsubstituted 5 to 6 membered heteroarylene. In embodiments, L^{A3} is not a unsubstituted 5 to 6 membered heteroarylene. In embodiments, L^{A3} is not unsubstituted divalent triazole. In embodiments, L^{A3} is not unsubstituted divalent 1H-1,2,3-triazole. In embodiments, L^{A3} is not unsubstituted divalent 2H-1,2,3-triazole. In embodiments, L^{A3} is not a divalent linker including one or more amino acids. In embodiments, L^{A3} is not a divalent linker consisting of amino acids. In embodiments, L^{A3} is not a divalent linker including an amino acid analog. In embodiments, L^{A3} is not a divalent linker including an amino acid mimetic. In embodiments, L^{A3} is not a divalent linker consisting of amino acid analogs. In embodiments, L^{A3} is not a divalent linker consisting of amino acid mimetics.

In embodiments, L^{A4} is not a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L^{A4} is not a substituted or unsubstituted 2 to 12 membered heteroalkylene. In embodiments, L^{A4} is not a substituted or unsubstituted 2 to 32 membered heteroalkylene. In embodiments, L^{A4} is not a bond. In embodiments, L^{A4} is not a divalent linker including one or more amino acids. In embodiments, L^{A4} is not a divalent linker consisting of amino acids. In embodiments, L^{A4} is not a divalent linker including an amino acid analog. In embodiments, L^{A4} is not a divalent linker including an amino acid mimetic. In embodiments, L^{A4} is not a divalent linker consisting of amino acid analogs. In embodiments, L^{A4} is not a divalent linker consisting of amino acid mimetics.

In embodiments, L^{A5} is not a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L^{A5} is not a substituted or unsubstituted 2 to 12 membered heteroalkylene. In embodiments, L^{A5} is not a substituted or unsubstituted 2 to 32 membered heteroalkylene. In embodiments, L^{A5} is not a bond. In embodiments, L^{A5} is not a divalent linker including one or more amino acids. In embodiments, L^{A5} is not a divalent linker consisting of amino acids. In embodiments, L^{A5} is not a divalent linker including an amino acid analog. In embodiments, L^{A5} is not a divalent linker including an amino acid mimetic. In embodiments, L^{A5} is not a divalent linker consisting of amino acid analogs. In embodiments, L^{A5} is not a divalent linker consisting of amino acid mimetics.

In embodiments, L^{A5} is not a divalent oligomer of ethylene oxide. In embodiments, L^{A5} is not a divalent polyethylene glycol. In embodiments, L^{A5} is not a divalent oligomer of ethylene oxide having 2 to 30 linear atoms (carbon and oxygen) between the two termini connecting to the remainder of the compound. In embodiments, L^{A5} is not a —(CH₂)₄C(O)NH—. In embodiments, L^{A5} is not a 2 to 8 membered substituted heteroalkylene. In embodiments, L^{A5} is not a 3 to 6 membered substituted heteroalkylene. In embodiments, L^{A5} is not a 5 to 6 membered substituted heteroalkylene. In embodiments, L^{A5} is not a 5 to 7 membered oxo substituted heteroalkylene. In embodiments, L^{A5} is not an unsubstituted C₁-C₆ alkylene.

In embodiments, L^{A4} is not a divalent oligomer of ethylene oxide. In embodiments, L^{A4} is not a divalent polyethylene glycol. In embodiments, L^{A4} is not a divalent oligomer of ethylene oxide having 2 to 30 linear atoms (carbon and oxygen) between the two termini connecting to the remainder of the compound. In embodiments, L^{A4} is not —(CH₂CH₂O)ₑCH₂CH₂— and eA is not an integer from 1 to 16. In embodiments, L^{A4} is not —(CH₂CH₂O)ₑCH₂— and eA is not an integer from 1 to 16. In embodiments, L^{A4} is not —(CH₂CH₂O)ₑ— and eA is not an integer from 1 to 16. In embodiments, eA is not an integer from 2 to 15. In embodiments, eA is not an integer from 3 to 14. In embodiments, eA is not an integer from 4 to 12. In embodiments, eA is not an integer from 5 to 10. In embodiments, eA is not an integer from 5 to 8. In embodiments, eA is not an integer from 6 to 7.

In embodiments, L^{A2} is not substituted (e.g., substituted with at least one substituent group, size-limited substituent group, or lower substituent group) or unsubstituted C₁-C₂₀ alkylene, substituted (e.g., substituted with at least one substituent group, size-limited substituent group, or lower substituent group) or unsubstituted 2 to 20 membered heteroalkylene, substituted (e.g., substituted with at least one substituent group, size-limited substituent group, or lower substituent group) or unsubstituted C₃-C₈ cycloalkylene, substituted (e.g., substituted with at least one substituent group, size-limited substituent group, or lower substituent group) or unsubstituted 3 to 8 membered heterocycloalkylene, substituted (e.g., substituted with at least one substituent group, size-limited substituent group, or lower substituent group) or unsubstituted C₆-C₁₀ arylene, or substituted (e.g., substituted with at least one substituent group, size-limited substituent group, or lower substituent group) or unsubstituted 5 to 10 membered heteroarylene. In embodiments, L^{A2} is not substituted (e.g., substituted with at least one substituent group, size-limited substituent group, or lower substituent group) or unsubstituted 3 to 8 membered heteroalkylene.

In embodiments, a substituted L^{A2} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{A2} is substituted with a plurality of groups selected from substituent groups, size-limited substituent groups, and lower substituent groups; each substituent group, size-limited substituent group, and/or lower substituent group may optionally be different. In embodiments, when L^{A2} is substituted, it is substituted with at least one substituent group. In embodiments, when L^{A2} is substituted, it is substituted with at least one size-limited substituent group. In embodiments, when L^{A2} is substituted, it is substituted with at least one lower substituent group.

In embodiments, L^{A3} is not a bond, substituted (e.g., substituted with at least one substituent group, size-limited substituent group, or lower substituent group) or unsubstituted C₁-C₂₀ alkylene, substituted (e.g., substituted with at least one substituent group, size-limited substituent group, or lower substituent group) or unsubstituted 2 to 20 membered heteroalkylene, substituted (e.g., substituted with at least one substituent group, size-limited substituent group, or lower substituent group) or unsubstituted C₃-C₈ cycloalkylene, substituted (e.g., substituted with at least one substituent group, size-limited substituent group, or lower substituent group) or unsubstituted 3 to 8 membered heterocycloalkylene, substituted (e.g., substituted with at least one substituent group, size-limited substituent group, or lower substituent group) or unsubstituted C₆-C₁₀ arylene, or substituted (e.g., substituted with at least one substituent group, size-limited substituent group, or lower substituent group) or unsubstituted 5 to 10 membered heteroarylene. In embodiments, L^{A3} is not a substituted (e.g., substituted with at least one substituent group, size-limited substituent group, or lower substituent group) or unsubstituted 5 to 10 membered heteroarylene.

In embodiments, a substituted L^{A3} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{A3} is substituted with a plurality of groups selected from substituent groups, size-limited substituent groups, and lower substituent groups; each substituent group, size-limited substituent group, and/or lower substituent group may optionally be different. In embodiments, when L^{A3} is substituted, it is substituted with at least one substituent group. In embodiments, when L^{A3} is substituted, it is substituted with at least one size-limited substituent group. In embodiments, when L^{A3} is substituted, it is substituted with at least one lower substituent group.

In embodiments, L^{A4} is not a bond, substituted (e.g., substituted with at least one substituent group, size-limited substituent group, or lower substituent group) or unsubstituted C₁-C₂₀ alkylene, substituted (e.g., substituted with at least one substituent group, size-limited substituent group, or lower substituent group) or unsubstituted 2 to 20 membered heteroalkylene, substituted (e.g., substituted with at least one substituent group, size-limited substituent group, or lower substituent group) or unsubstituted C₃-C₈ cycloalkylene, substituted (e.g., substituted with at least one substituent group, size-limited substituent group, or lower substituent group) or unsubstituted 3 to 8 membered heterocycloalkylene, substituted (e.g., substituted with at least one substituent group, size-limited substituent group, or lower substituent group) or unsubstituted C₆-C₁₀ arylene, or substituted (e.g., substituted with at least one substituent group, size-limited substituent group, or lower substituent group) or unsubstituted 5 to 10 membered heteroarylene. In embodiments, L^{A4} is not a substituted (e.g., substituted with at least one substituent group, size-limited substituent group, or lower substituent group) or unsubstituted 2 to 12 membered heteroalkylene. In embodiments, L^{A4} is not a substituted (e.g., substituted with at least one substituent group, size-limited substituent group, or lower substituent group) or unsubstituted 2 to 32 membered heteroalkylene.

In embodiments, a substituted L^{A4} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{A4} is substituted with a plurality of groups selected from substituent groups, size-limited substituent groups, and lower substituent groups; each substituent group, size-limited substituent group, and/or lower substituent group may optionally be different. In embodiments, when L^{A4} is substituted, it is substituted with at least one substituent group. In embodiments, when L^{A4} is substituted, it is substituted with at least one size-limited substituent group. In embodiments, when L^{A4} is substituted, it is substituted with at least one lower substituent group.

In embodiments, L^{A5} is not a bond, substituted (e.g., substituted with at least one substituent group, size-limited substituent group, or lower substituent group) or unsubstituted C₁-C₂₀ alkylene, substituted (e.g., substituted with at least one substituent group, size-limited substituent group, or lower substituent group) or unsubstituted 2 to 20 membered heteroalkylene, substituted (e.g., substituted with at least one substituent group, size-limited substituent group, or lower substituent group) or unsubstituted C₃-C₈ cycloalkylene, substituted (e.g., substituted with at least one substituent group, size-limited substituent group, or lower substituent group) or unsubstituted 3 to 8 membered heterocycloalkylene, substituted (e.g., substituted with at least one substituent group, size-limited substituent group, or lower substituent group) or unsubstituted C₆-C₁₀ arylene, or substituted (e.g., substituted with at least one substituent group, size-limited substituent group, or lower substituent group) or unsubstituted 5 to 10 membered heteroarylene. In embodiments, L^{A5} is not a substituted (e.g., substituted with at least one substituent group, size-limited substituent group, or lower substituent group) or unsubstituted 2 to 12 membered heteroalkylene. In embodiments, L^{A5} is not a substituted (e.g., substituted with at least one substituent group, size-limited substituent group, or lower substituent group) or unsubstituted 2 to 32 membered heteroalkylene.

In embodiments, a substituted L^{A5} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{A5} is substituted with a plurality of groups selected from substituent groups, size-limited substituent groups, and lower substituent groups; each substituent group, size-limited substituent group, and/or lower substituent group may optionally be different. In embodiments, when L^{A5} is substituted, it is substituted with at least one substituent group. In embodiments, when L^{A5} is substituted, it is substituted with at least one size-limited substituent group. In embodiments, when L^{A5} is substituted, it is substituted with at least one lower substituent group.

In embodiments, L^{A5} is not a 2 to 8 membered substituted (e.g., substituted with at least one substituent group, size-limited substituent group, or lower substituent group) heteroalkylene. In embodiments, L^{A5} is not a 3 to 6 membered substituted (e.g., substituted with at least one substituent group, size-limited substituent group, or lower substituent group) heteroalkylene. In embodiments, L^{A5} is not a 5 to 6 membered substituted (e.g., substituted with at least one substituent group, size-limited substituent group, or lower substituent group) heteroalkylene. In embodiments, L^{A5} is not a 5 to 7 membered oxo substituted (e.g., substituted with at least one substituent group, size-limited substituent group, or lower substituent group) heteroalkylene.

In embodiments, the linker is not formed by a conjugation or bioconjugation reaction combining a first reactant moiety covalently bonded to the rapamycin or rapamycin analog and a second reactant moiety covalently bonded to the active site mTOR inhibitor.

In embodiments, the compound does not compete with rapamycin for binding to mTORC1. In embodiments, the compound does not bind an overlapping region of mTORC1 with the binding region of rapamycin. In embodiments, the compound does not compete with ATP for binding to mTOR. In embodiments, the compound does not compete with ATP for binding to mTORC1. In embodiments, the compound does not compete with rapamycin and ATP for binding to mTORC1.

In embodiments, the compound is not an mTORC1 specific inhibitor. In embodiments, the compound does not have a slow off-rate from mTORC1. In embodiments, the compound does not have an off-rate of slower than 0.1 per minute. In embodiments, the compound does not have an off-rate of slower than 0.01 per minute. In embodiments, the compound does not have an off-rate of slower than 0.001 per minute. In embodiments, the compound does not have an off-rate of slower than 0.0001 per minute.

In embodiments, the compound is not

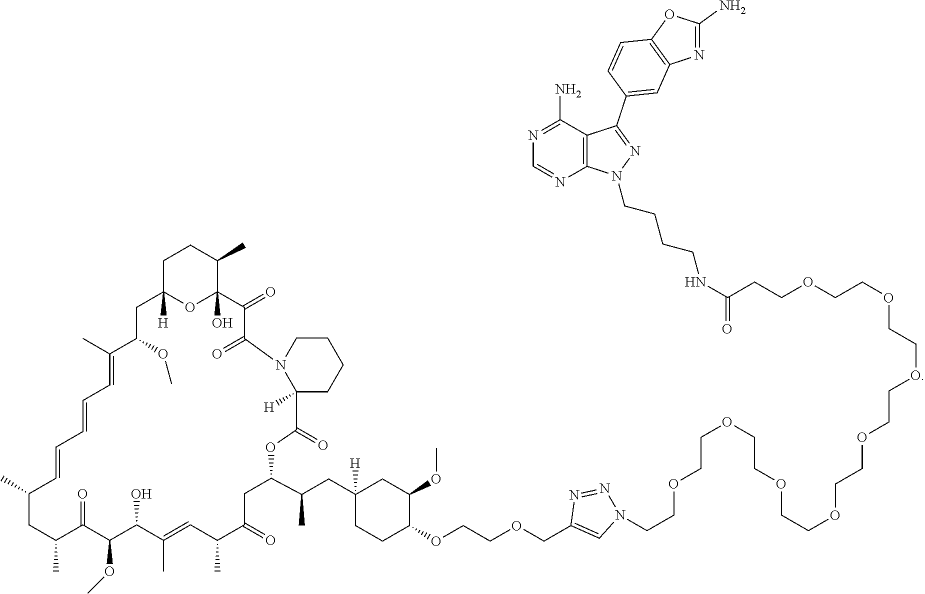 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCOCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

In embodiments, the compound is not

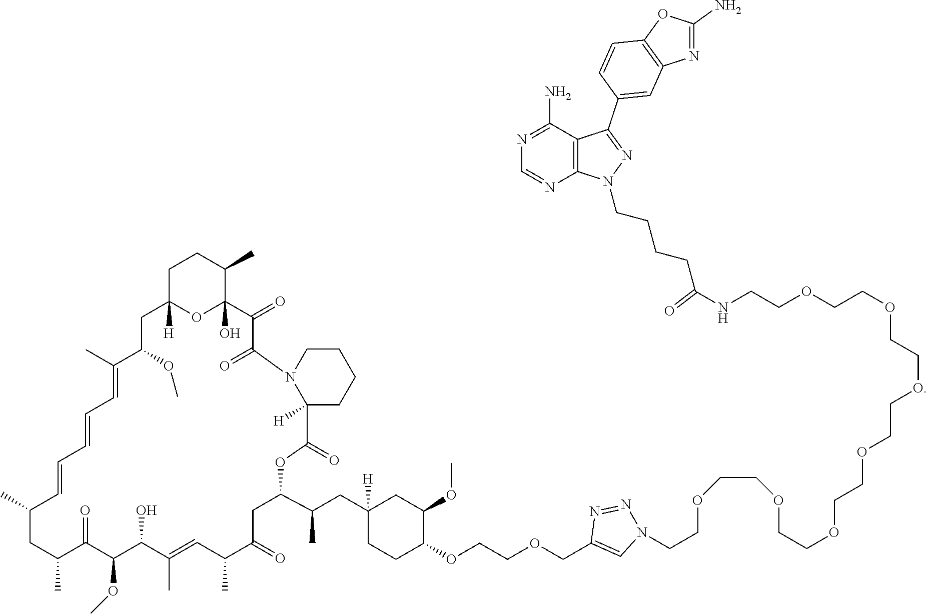 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCOCc3cn(CCOCCOCCOCCOCCOCCOCCOCCNC(=O)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

In embodiments, compound is not

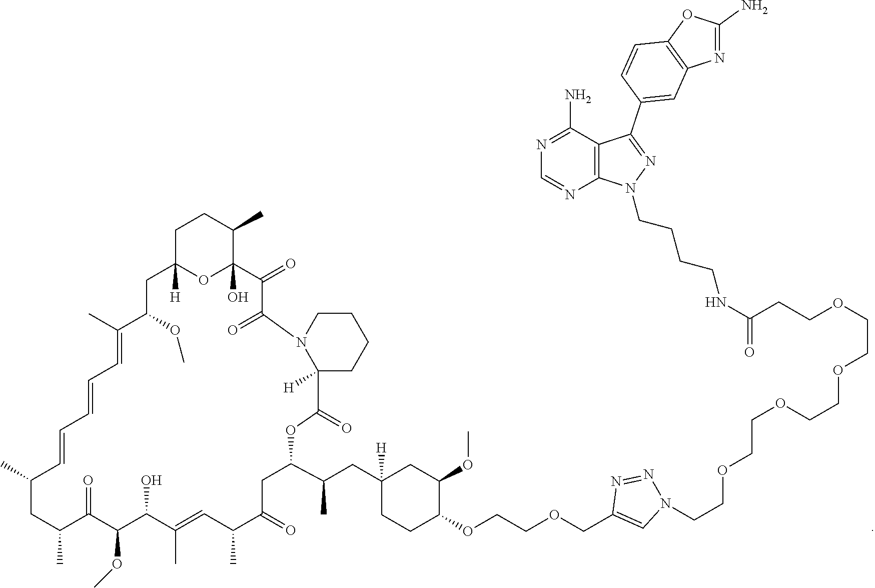 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCOCc3cn(CCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

In embodiments, the compound is not

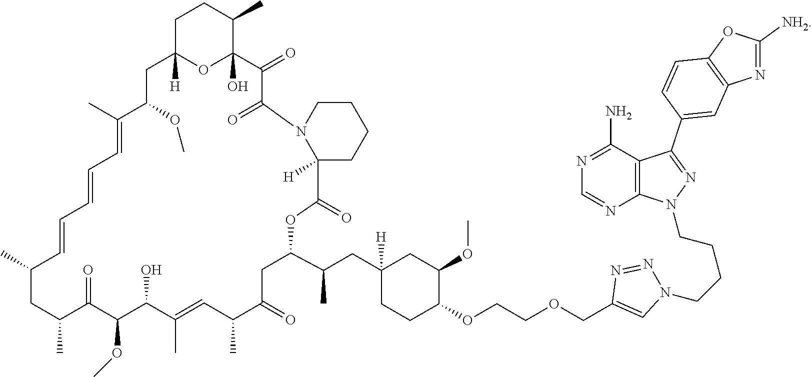 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCOCc3cn(CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

In embodiments, the active site mTOR inhibitor is not a monovalent MLN0128.

In embodiments, the active site mTOR inhibitor is not

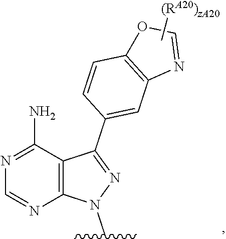

wherein R^{A20} is as described herein, including in embodiments. In embodiments, zA20 is not an integer from 0 to 4. In embodiments, the active site mTOR inhibitor is not

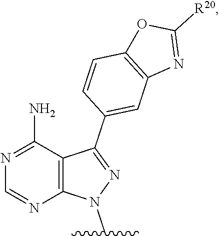

wherein R^{A20} is as described herein, including in embodiments. In embodiments, the active site mTOR inhibitor is not

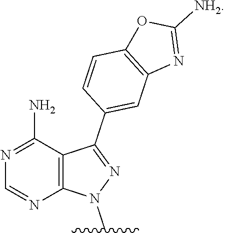 [SMILES: *n1nc(-c2ccc3oc(N)nc3c2)c2c(N)ncnc21]

In embodiments, the active site mTOR inhibitor is not

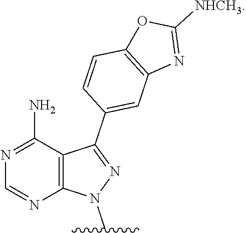 [SMILES: *n1nc(-c2ccc3oc(NC)nc3c2)c2c(N)ncnc21]

In embodiments, the active site mTOR inhibitor is not

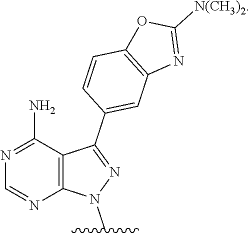 [SMILES: *n1nc(-c2ccc3oc(N(C)C)nc3c2)c2c(N)ncnc21]

In embodiments, the active site mTOR inhibitor is not

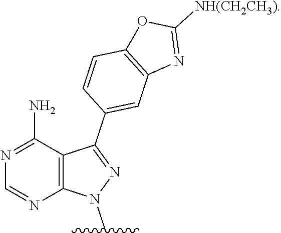

In embodiments, the active site mTOR inhibitor is not

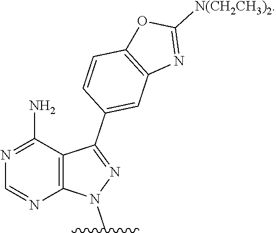

In embodiments, the active site mTOR inhibitor is not

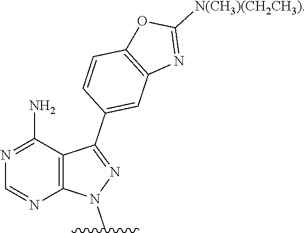

In embodiments, the active site mTOR inhibitor is not

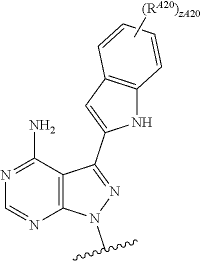

wherein R^{A20} is as described herein, including in embodiments. zA20 is an integer from 0 to 5. In embodiments, the active site mTOR inhibitor is not

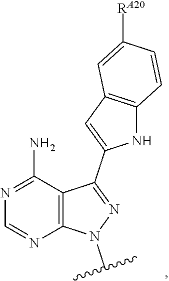

wherein R^{A20} is as described herein. In embodiments, the active site mTOR inhibitor is not

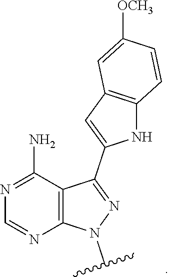 [SMILES: *n1nc(-c2cc3cc(OC)ccc3[nH]2)c2c(N)ncnc21]

In embodiments, the active site mTOR inhibitor is not

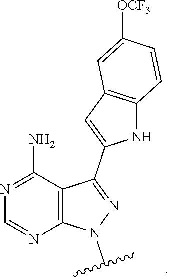 [SMILES: *n1nc(-c2cc3cc(OC(F)(F)F)ccc3[nH]2)c2c(N)ncnc21]

In embodiments, the active site mTOR inhibitor is not

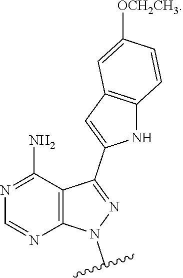 [SMILES: *n1nc(-c2cc3cc(OCC)ccc3[nH]2)c2c(N)ncnc21]

In embodiments, the active site mTOR inhibitor is not

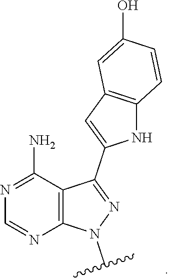 [SMILES: *n1nc(-c2cc3cc(O)ccc3[nH]2)c2c(N)ncnc21]

Without being limited by mechanism, the compound may not include an active site mTOR inhibitor that results in a preferential binding of the compound to mTORC1 over mTORC2 of at least 1.1-fold (e.g., at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 10000, 20000, 30000, 40000, 50000, 60000, 70000, 80000, 90000, 100000, 100000, 200000, 300000, 400000, 500000, 600000, 700000, 800000, 900000, or 1000000 fold). Without being limited by mechanism, the compound may not include an active site mTOR inhibitor that results in a preferential inhibition of mTORC1 over mTORC2 by the compound of at least 1.1-fold (e.g., at least 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 10000, 20000, 30000, 40000, 50000, 60000, 70000, 80000, 90000, 100000, 100000, 200000, 300000, 400000, 500000, 600000, 700000, 800000, 900000, or 1000000 fold).

In embodiments, the compound is not:

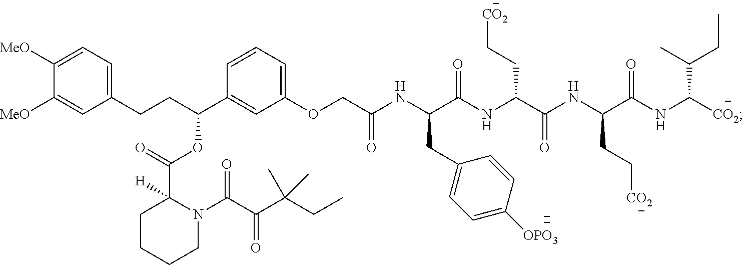

or an analog thereof.

In embodiments, the compound is not:

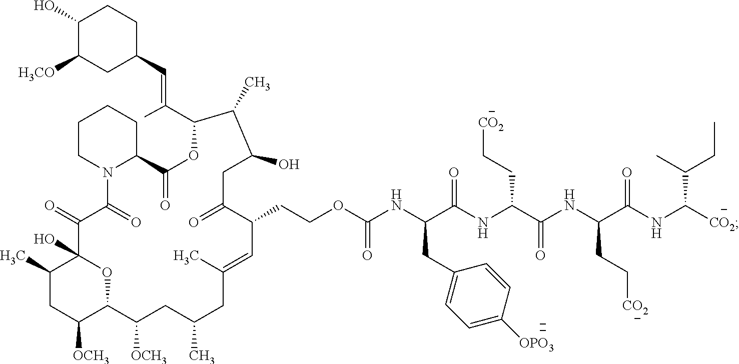

or an analog thereof.

In embodiments, the compound is not:

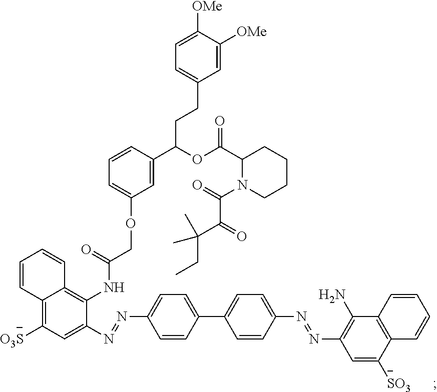

or an analog thereof.

In embodiments, the compound is not:

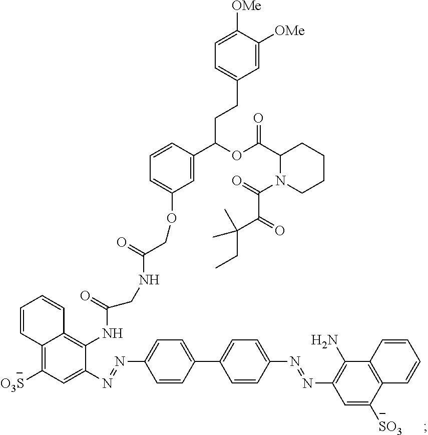

or an analog thereof.

In embodiments, the compound is not:

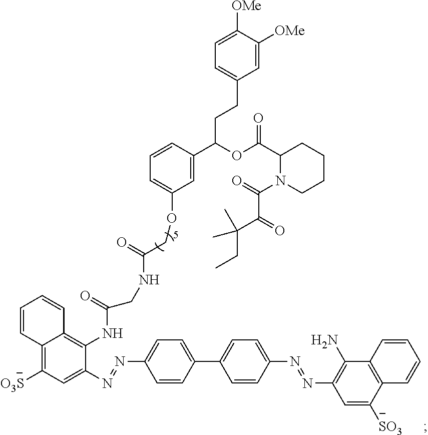

or an analog thereof.

In embodiments, the compound is not:

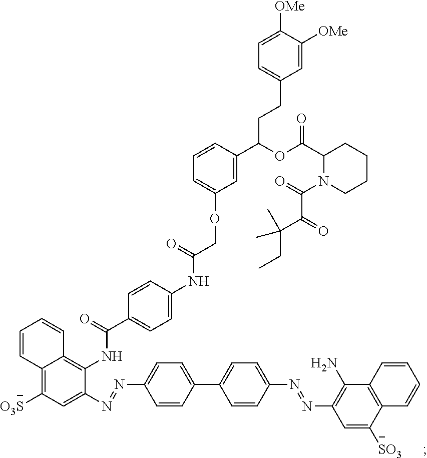

or an analog thereof.

In embodiments, the compound is not:

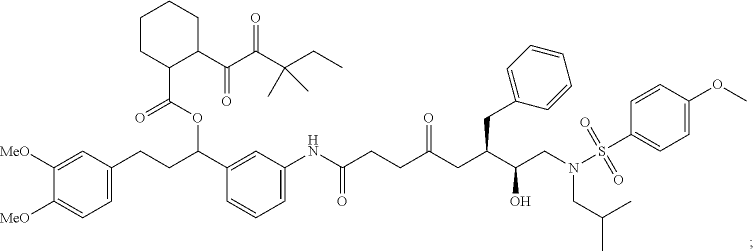 [SMILES: CCC(C)(C)C(=O)C(=O)C1CCCCC1C(=O)OC(CCc1ccc(OC)c(OC)c1)c1cccc(NC(=O)CCC(=O)C[C@@H](Cc2ccccc2)[C@H](O)CN(CC(C)C)S(=O)(=O)c2ccc(OC)cc2)c1]

or an analog thereof.

In embodiments, the compound is not:

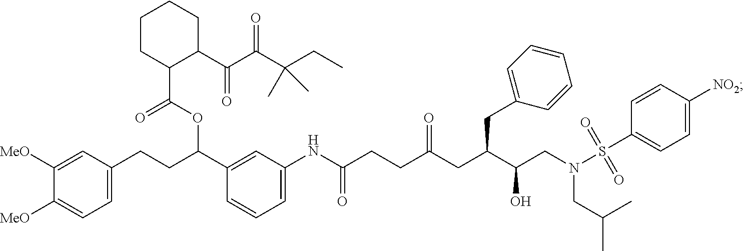 [SMILES: CCC(C)(C)C(=O)C(=O)C1CCCCC1C(=O)OC(CCc1ccc(OC)c(OC)c1)c1cccc(NC(=O)CCC(=O)C[C@@H](Cc2ccccc2)[C@H](O)CN(CC(C)C)S(=O)(=O)c2ccc([N+](=O)[O-])cc2)c1]

or an analog thereof.

In embodiments, the compound is not:

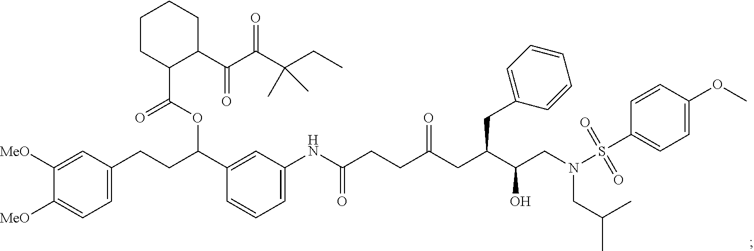 [SMILES: CCC(C)(C)C(=O)C(=O)C1CCCCC1C(=O)OC(CCc1ccc(OC)c(OC)c1)c1cccc(NC(=O)CCC(=O)C[C@@H](Cc2ccccc2)[C@H](O)CN(CC(C)C)S(=O)(=O)c2ccc(OC)cc2)c1]

or an analog thereof.

In embodiments, the compound is not:

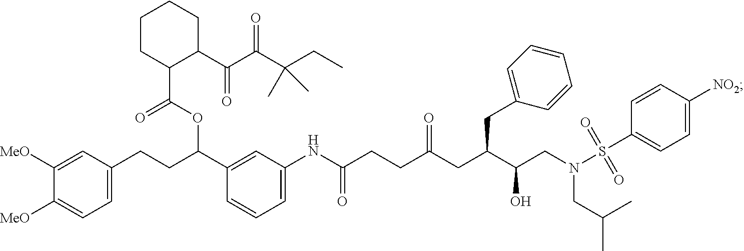 [SMILES: CCC(C)(C)C(=O)C(=O)C1CCCCC1C(=O)OC(CCc1ccc(OC)c(OC)c1)c1cccc(NC(=O)CCC(=O)C[C@@H](Cc2ccccc2)[C@H](O)CN(CC(C)C)S(=O)(=O)c2ccc([N+](=O)[O-])cc2)c1]

or an analog thereof.

In embodiments, the compound is not:

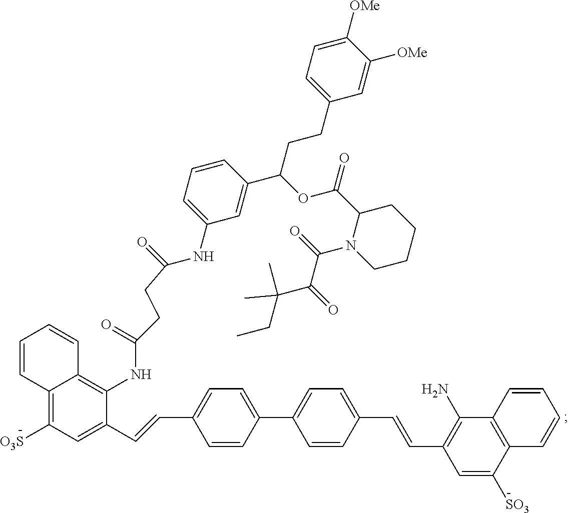

or an analog thereof.

In embodiments, the compound is not:

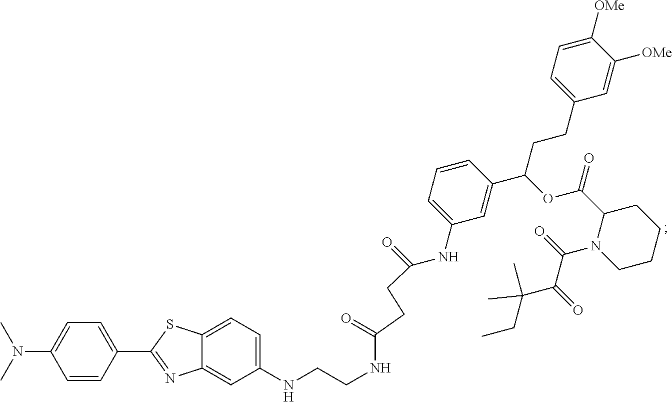 [SMILES: CCC(C)(C)C(=O)C(=O)N1CCCCC1C(=O)OC(CCc1ccc(OC)c(OC)c1)c1cccc(NC(=O)CCC(=O)NCCNc2ccc3sc(-c4ccc(N(C)C)cc4)nc3c2)c1]

or an analog thereof.

In embodiments, the compound is not:

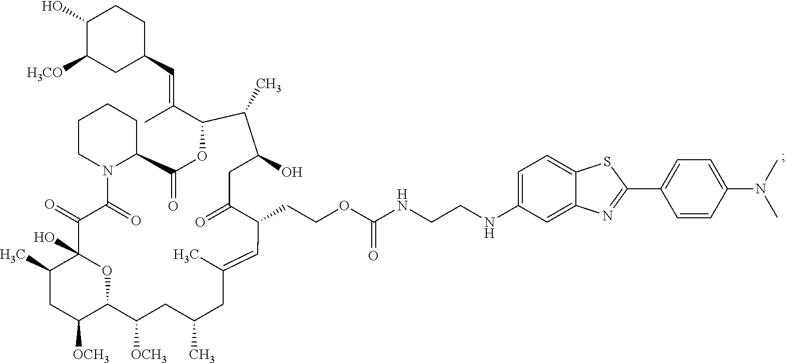

or an analog thereof.

In embodiments, the compound is not:

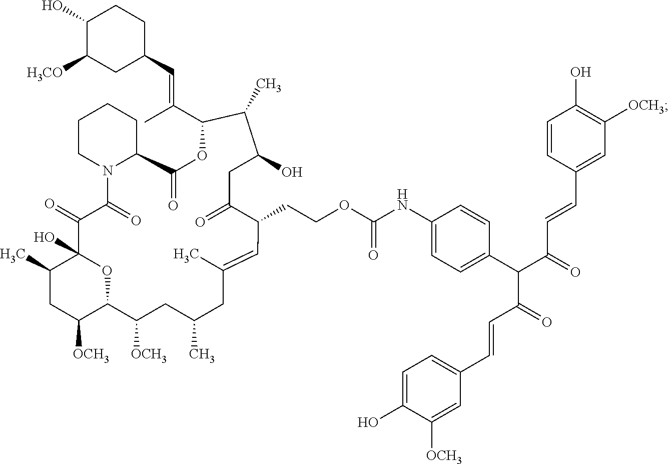 [SMILES: COc1cc(/C=C/C(=O)C(C(=O)/C=C/c2ccc(O)c(OC)c2)c2ccc(NC(=O)OCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/[C@@H]5CC[C@@H](O)[C@H](OC)C5)[C@H](C)[C@@H](O)CC3=O)[C@H](C)C[C@@H]4OC)cc2)ccc1O]

or an analog thereof.

In embodiments, the compound is not:

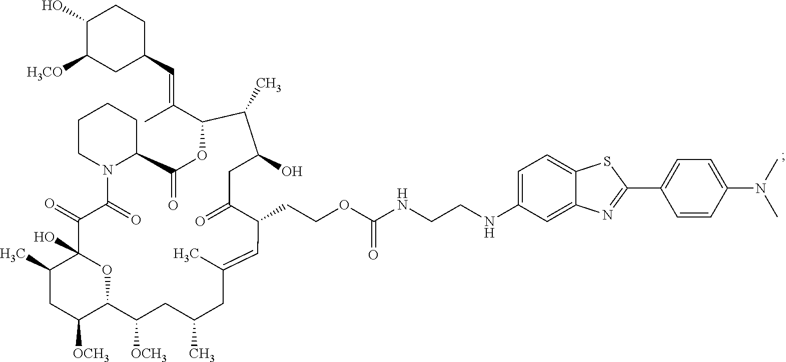

or an analog thereof.

In embodiments, the compound is not:

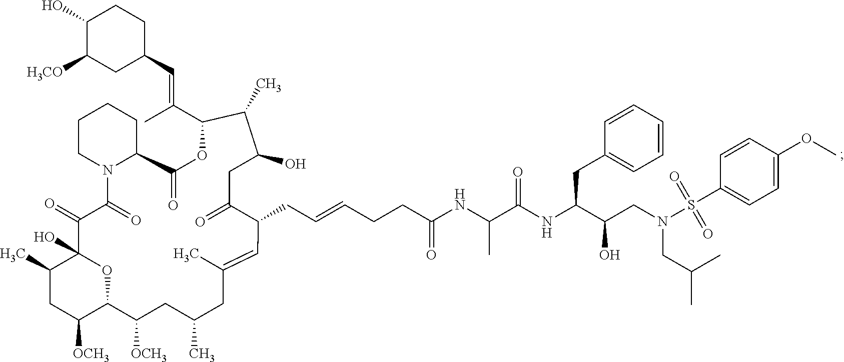

or an analog thereof.

In embodiments, the compound is not:

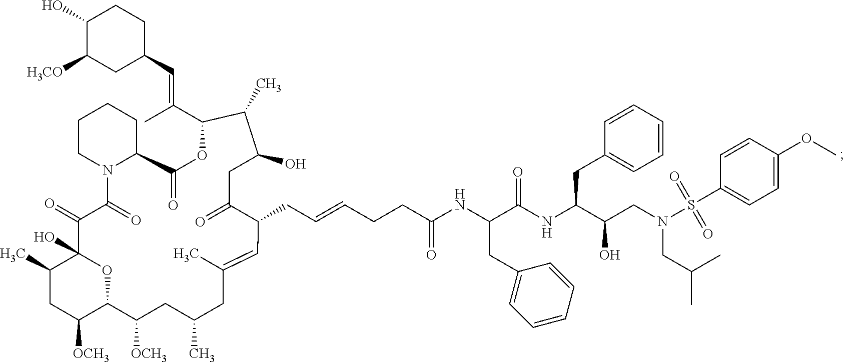

or an analog thereof.

In embodiments, the compound is not:

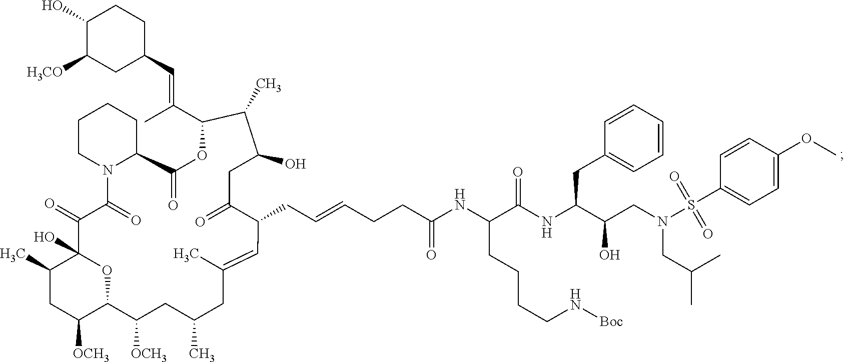

or an analog thereof.

In embodiments, the compound is not:

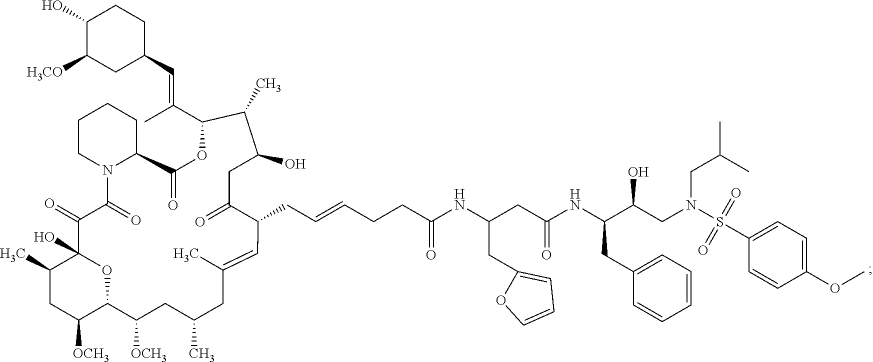

an analog thereof.

In embodiments, the compound is not:

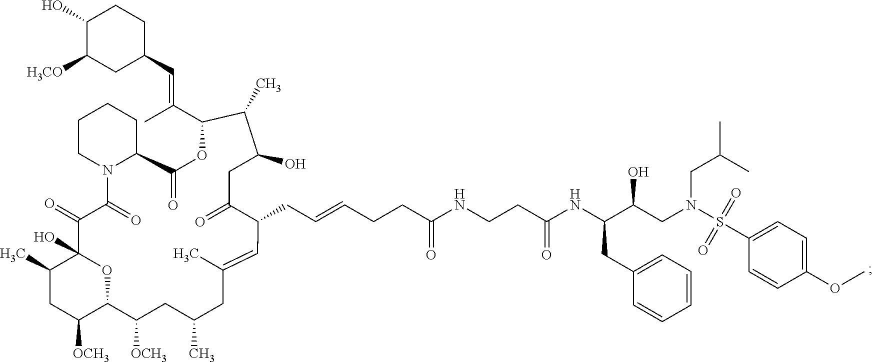

or an analog thereof.

In embodiments, the compound is not:

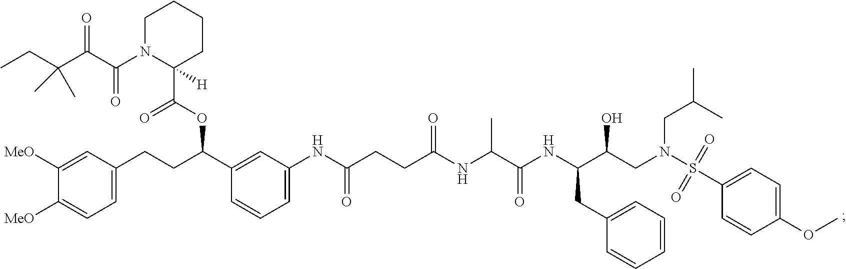 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(C)C(=O)N[C@H](Cc2ccccc2)[C@@H](O)CN(CC(C)C)S(=O)(=O)c2ccc(OC)cc2)c1]

or an analog thereof.

In embodiments, the compound is not:

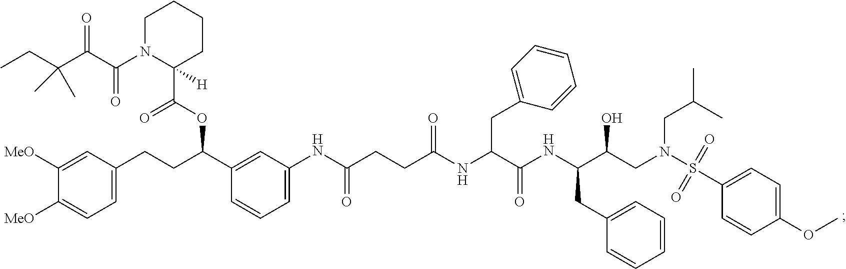 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(Cc2ccccc2)C(=O)N[C@H](Cc2ccccc2)[C@@H](O)CN(CC(C)C)S(=O)(=O)c2ccc(OC)cc2)c1]

or an analog thereof.

In embodiments, the compound is not:

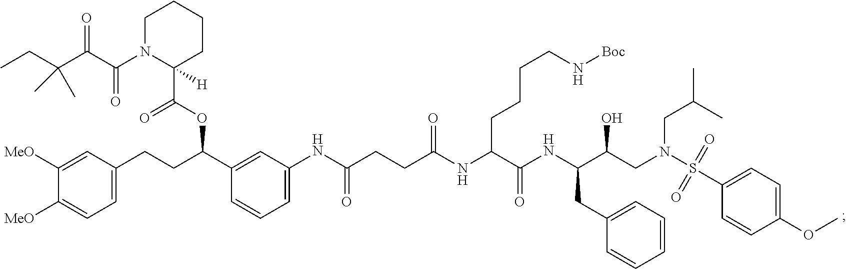 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(CCCCNC(=O)OC(C)(C)C)C(=O)N[C@H](Cc2ccccc2)[C@@H](O)CN(CC(C)C)S(=O)(=O)c2ccc(OC)cc2)c1]

or an analog thereof.

In embodiments, the compound is not:

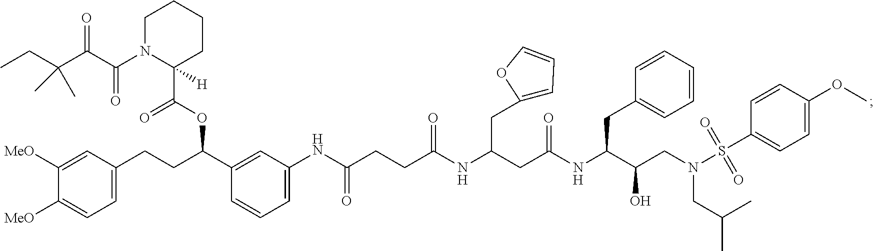 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(CC(=O)N[C@@H](Cc2ccccc2)[C@H](O)CN(CC(C)C)S(=O)(=O)c2ccc(OC)cc2)Cc2ccco2)c1]

or an analog thereof.

In embodiments, the compound is not:

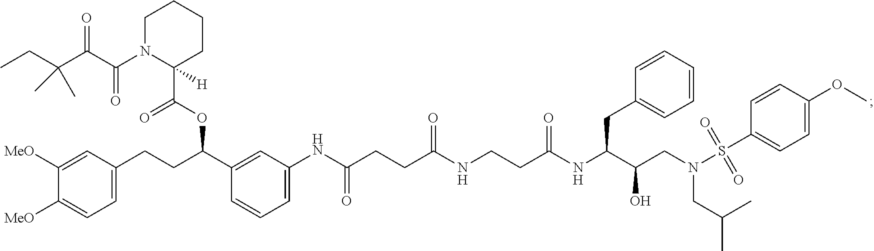 [SMILES: CCC(C)(C)C(=O)C(=O)N1CCCC[C@H]1C(=O)O[C@H](CCc1ccc(OC)c(OC)c1)c1cccc(NC(=O)CCC(=O)NCCC(=O)N[C@@H](Cc2ccccc2)[C@H](O)CN(CC(C)C)S(=O)(=O)c2ccc(OC)cc2)c1]

or an analog thereof.

In embodiments, the compound is not:

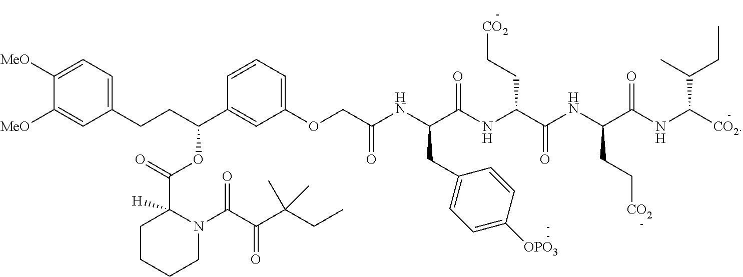

In embodiments, the compound is not:

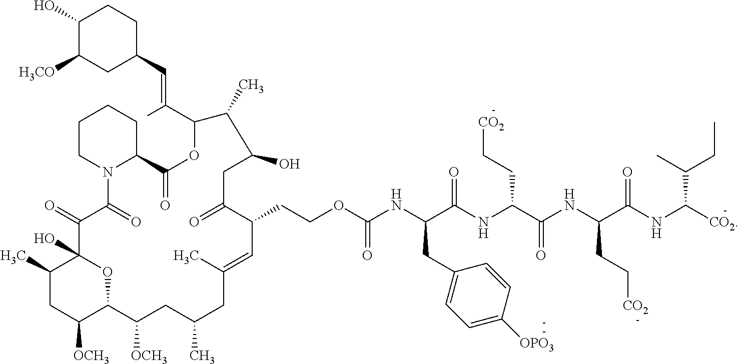

In embodiments, the compound is not:

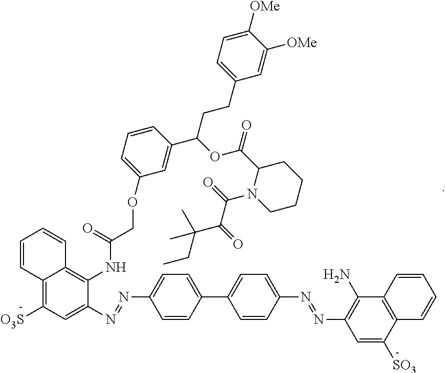

In embodiments, the compound is not:

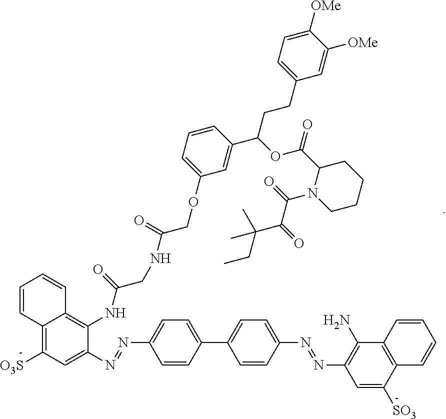

In embodiments, the compound is not:

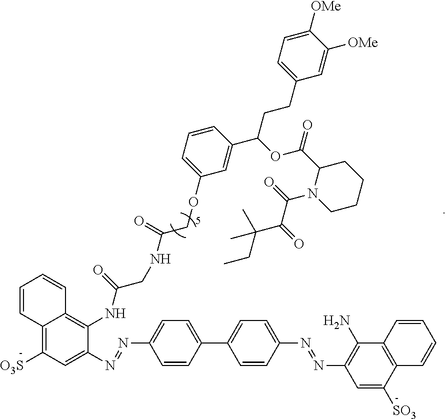

In embodiments, the compound is not:

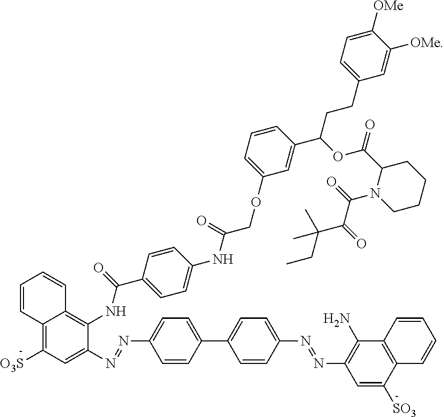

In embodiments, the compound is not:

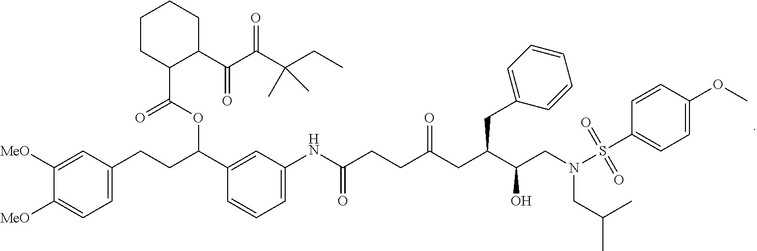 [SMILES: CCC(C)(C)C(=O)C(=O)C1CCCCC1C(=O)OC(CCc1ccc(OC)c(OC)c1)c1cccc(NC(=O)CCC(=O)C[C@@H](Cc2ccccc2)[C@H](O)CN(CC(C)C)S(=O)(=O)c2ccc(OC)cc2)c1]

In embodiments, the compound is not:

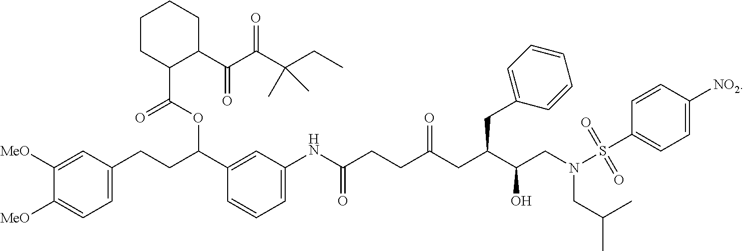 [SMILES: CCC(C)(C)C(=O)C(=O)C1CCCCC1C(=O)OC(CCc1ccc(OC)c(OC)c1)c1cccc(NC(=O)CCC(=O)C[C@@H](Cc2ccccc2)[C@H](O)CN(CC(C)C)S(=O)(=O)c2ccc([N+](=O)[O-])cc2)c1]

In embodiments, the compound is not:

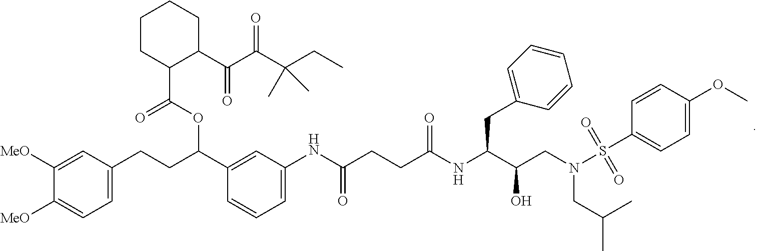 [SMILES: CCC(C)(C)C(=O)C(=O)C1CCCCC1C(=O)OC(CCc1ccc(OC)c(OC)c1)c1cccc(NC(=O)CCC(=O)N[C@@H](Cc2ccccc2)[C@H](O)CN(CC(C)C)S(=O)(=O)c2ccc(OC)cc2)c1]

In embodiments, the compound is not:

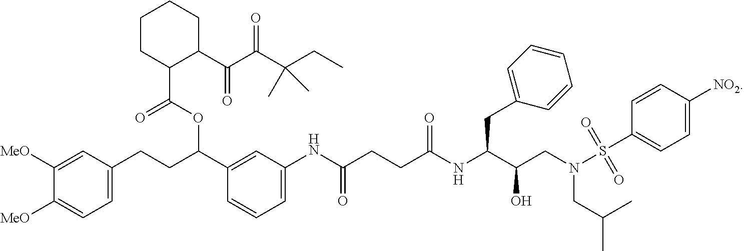 [SMILES: CCC(C)(C)C(=O)C(=O)C1CCCCC1C(=O)OC(CCc1ccc(OC)c(OC)c1)c1cccc(NC(=O)CCC(=O)N[C@@H](Cc2ccccc2)[C@H](O)CN(CC(C)C)S(=O)(=O)c2ccc([N+](=O)[O-])cc2)c1]

In embodiments, the compound is not:

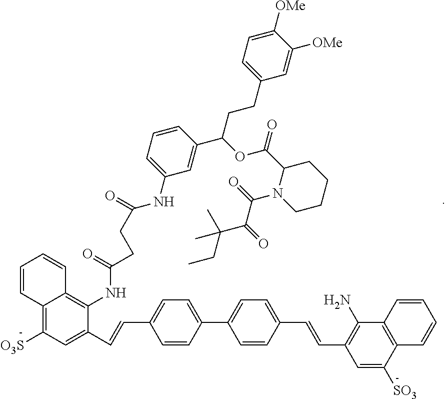

In embodiments, the compound is not:

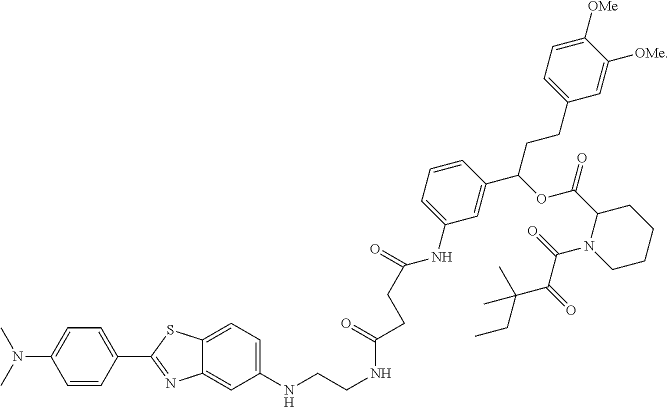 [SMILES: CCC(C)(C)C(=O)C(=O)N1CCCCC1C(=O)OC(CCc1ccc(OC)c(OC)c1)c1cccc(NC(=O)CCC(=O)NCCNc2ccc3sc(-c4ccc(N(C)C)cc4)nc3c2)c1]

In embodiments, the compound is not:

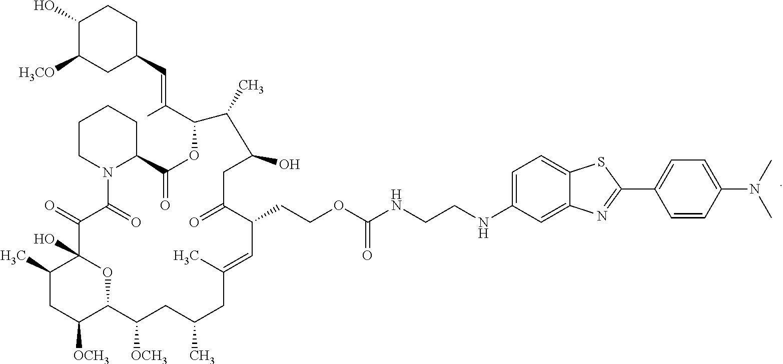

In embodiments, the compound is not:

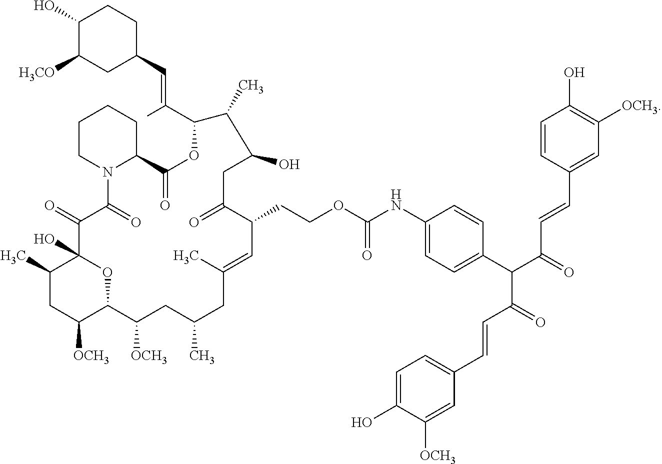 [SMILES: COc1cc(/C=C/C(=O)C(C(=O)/C=C/c2ccc(O)c(OC)c2)c2ccc(NC(=O)OCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/[C@@H]5CC[C@@H](O)[C@H](OC)C5)[C@H](C)[C@@H](O)CC3=O)[C@H](C)C[C@@H]4OC)cc2)ccc1O]

In embodiments, the compound is not:

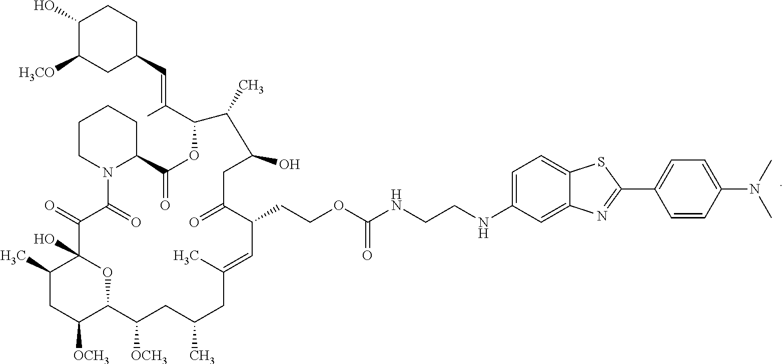

In embodiments, the compound is not:

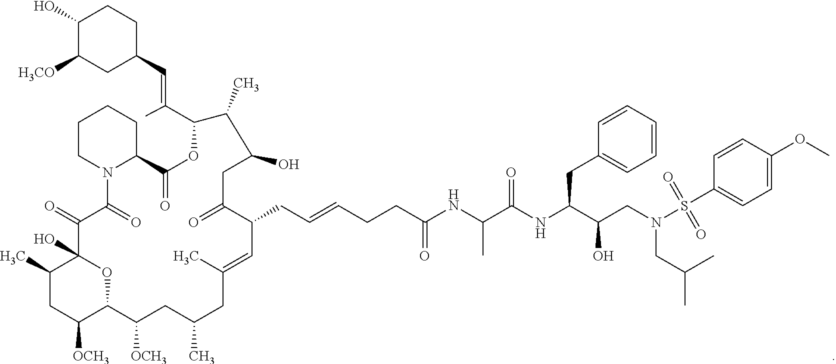

In embodiments, the compound is not:

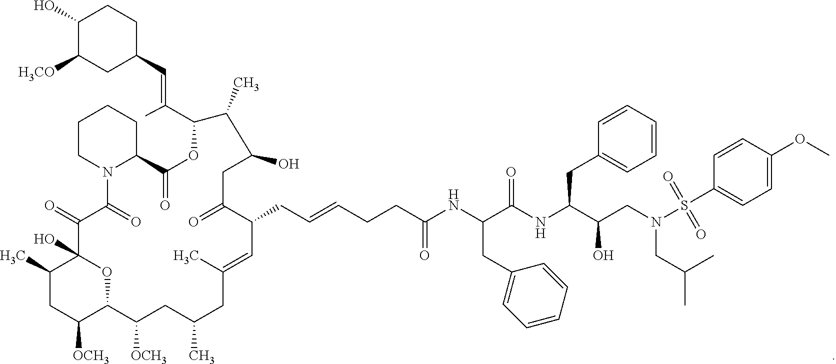

In embodiments, the compound is not:

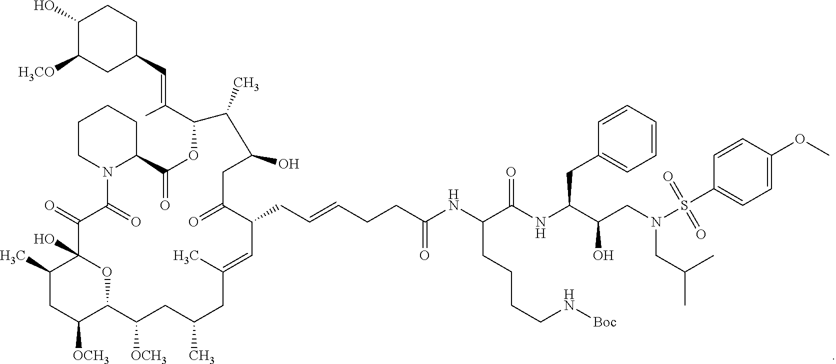

In embodiments, the compound is not:

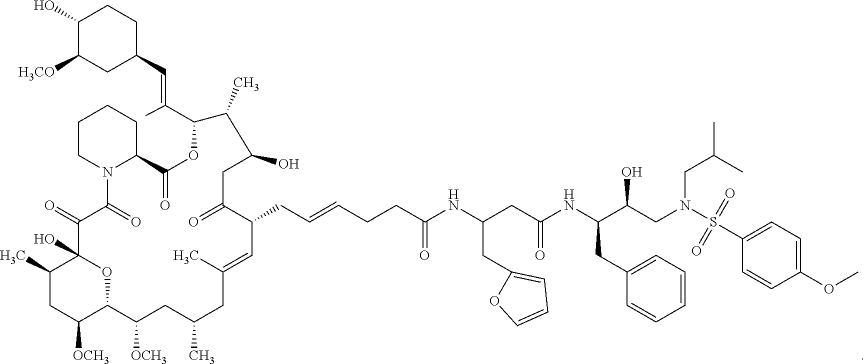

In embodiments, the compound is not:

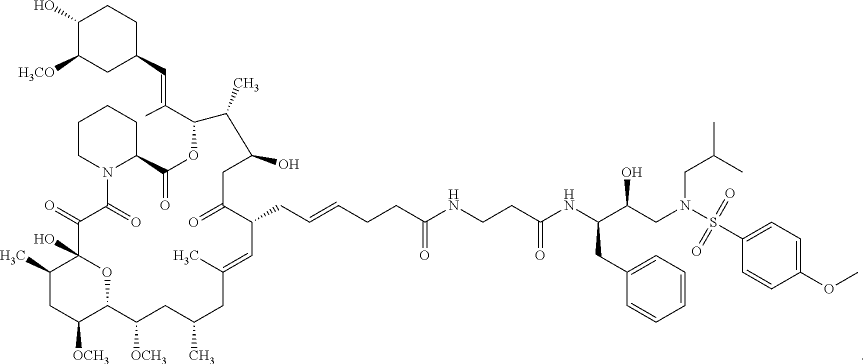

In embodiments, the compound is not:

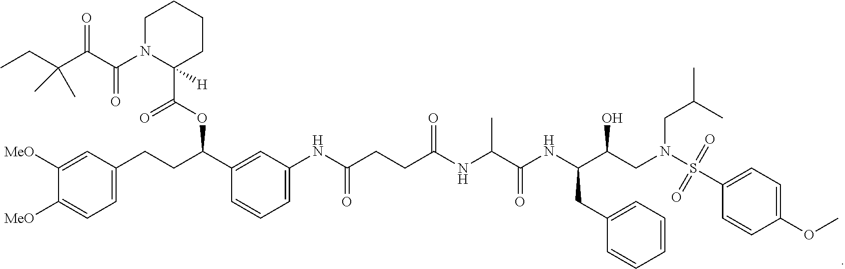 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(C)C(=O)N[C@H](Cc2ccccc2)[C@@H](O)CN(CC(C)C)S(=O)(=O)c2ccc(OC)cc2)c1]

In embodiments, the compound is not:

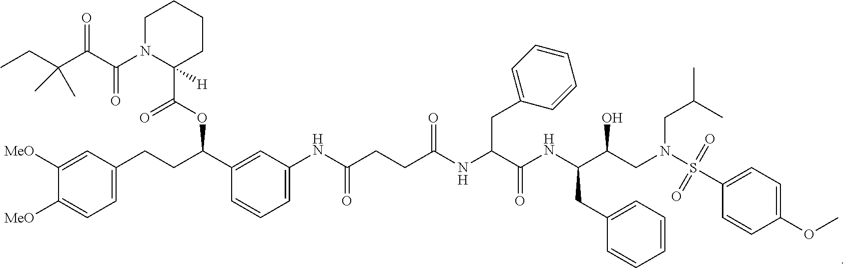 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(Cc2ccccc2)C(=O)N[C@H](Cc2ccccc2)[C@@H](O)CN(CC(C)C)S(=O)(=O)c2ccc(OC)cc2)c1]

In embodiments, the compound is not:

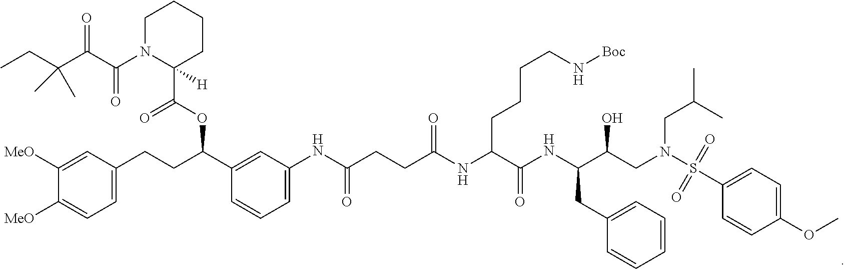 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(CCCCNC(=O)OC(C)(C)C)C(=O)N[C@H](Cc2ccccc2)[C@@H](O)CN(CC(C)C)S(=O)(=O)c2ccc(OC)cc2)c1]

In embodiments, the compound is not:

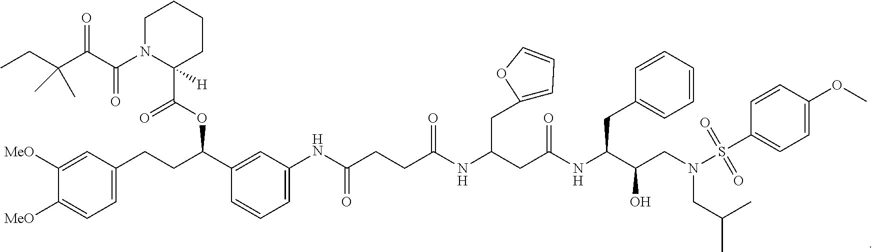 [SMILES: CCC(C)(C)C(=O)C(=O)N1CCCC[C@H]1C(=O)O[C@H](CCc1ccc(OC)c(OC)c1)c1cccc(NC(=O)CCC(=O)NC(CC(=O)N[C@@H](Cc2ccccc2)[C@H](O)CN(CC(C)C)S(=O)(=O)c2ccc(OC)cc2)Cc2ccco2)c1]

In embodiments, the compound is not:

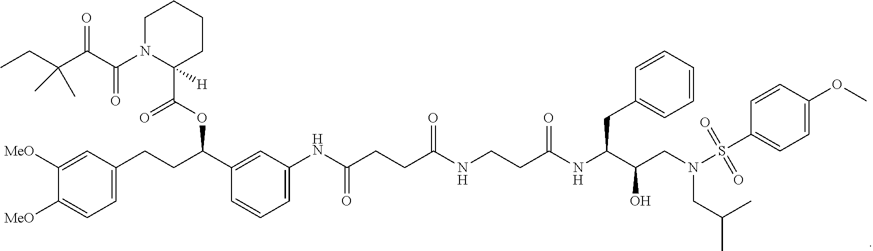 [SMILES: CCC(C)(C)C(=O)C(=O)N1CCCC[C@H]1C(=O)O[C@H](CCc1ccc(OC)c(OC)c1)c1cccc(NC(=O)CCC(=O)NCCC(=O)N[C@@H](Cc2ccccc2)[C@H](O)CN(CC(C)C)S(=O)(=O)c2ccc(OC)cc2)c1]

In embodiments, the compound does not bind mTORC1. In embodiments, the compound does not bind mTOR. In embodiments, the immunophilin binding moiety is not rapamycin or an analog thereof. In embodiments, the immunophilin binding moiety is not rapamycin.

In embodiments, the compound residence time in cells is from 1 to 24 hours. In embodiments, the compound residence time in cells is from 1 to 12 hours. In embodiments, the compound residence time in cells is from 12 to 24 hours. In embodiments, the compound residence time in cells is 1, 2, 3, 4, 5, 6, 7, 8, 9, 10, 11, 12, 13, 14, 15, 16, 17, 18, 19, 20, 21, 22, 23, or 24 hours. In embodiments, the compound residence time in cells is about 1, 2, 3, 4, 5, 6, 7, 8, 9, 10, 11, 12, 13, 14, 15, 16, 17, 18, 19, 20, 21, 22, 23, or 24 hours. In embodiments, the compound residence time in cells is at least 1, 2, 3, 4, 5, 6, 7, 8, 9, 10, 11, 12, 13, 14, 15, 16, 17, 18, 19, 20, 21, 22, 23, or 24 hours. In embodiments, the compound including a monovalent kinase inhibitor, a monovalent pseudokinase inhibitor, a monovalent GTPase inhibitor, a monovalent histone-modifying enzyme inhibitor, or monovalent anti-viral agent has a residence time in cells that is at least 1.1 fold (e.g., at least 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 fold) greater than the residence time of the corresponding kinase inhibitor, a pseudokinase inhibitor, a GTPase inhibitor, a histone-modifying enzyme inhibitor, or anti-viral agent.

### III. Pharmaceutical Compositions

In an aspect is provided a pharmaceutical composition including a pharmaceutically acceptable excipient and a compound as provided herein, including embodiments thereof.

In embodiments, the pharmaceutical composition includes an effective amount of the compound. In embodiments, the pharmaceutical composition includes a therapeutically effective amount of the compound.

### IV. Methods of Use

In one aspect is provided a method of treating a disease associated with aberrant enzyme activity in a subject in need of such treatment, including administering a compound as provided herein, including embodiments thereof, to the subject.

In embodiments, the enzyme activity is a kinase activity (e.g., a kinase described herein).

In embodiments, the kinase activity is in the CNS of the subject (e.g., brain).

In one aspect is provided a method of treating a disease in a subject in need of such treatment, including administering a compound as provided herein, including embodiments thereof, to the subject, wherein the disease is cancer or a neurodegenerative disease. In one aspect is provided a method of treating a disease in a subject in need of such treatment, including administering a compound as provided herein, including embodiments thereof, to the subject, wherein the disease is a viral disease.

In embodiments, the disease is cancer.

In embodiments, the cancer is glioblastoma or glioma.

In embodiments, the disease is a neurodegenerative disease. In embodiments, the neurodegenerative disease is not Alzheimer's Disease. In embodiments, the compound is not an amyloid β aggregation inhibitor. In embodiments, the compound does not include a monovalent amyloid β aggregation inhibitor.

In embodiments, the viral disease is not human immunodeficiency (HIV) virus. In embodiments, the compound is not an HIV inhibitor. In embodiments, the compound is not an HIV protease inhibitor. In embodiments, the compound is not a viral protease inhibitor. In embodiments, the compound does not include an HIV inhibitor. In embodiments, the compound does not include an HIV protease inhibitor. In embodiments, the compound does not include a viral protease inhibitor.

In embodiments the neurodegenerative disease is Parkinson's Disease. In embodiments the neurodegenerative disease is Amyotrophic lateral sclerosis (ALS). In embodiments the neurodegenerative disease is Alzheimer's Disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. Embodiments

Embodiment P1. A compound having the formula:

A-L¹-R¹;

wherein
A is an immunophilin-binding moiety;
L¹ is a bond or a covalent linker; and
R¹ is a kinase inhibitor, a pseudokinase inhibitor, a GTPase inhibitor, a histone-modifying enzyme inhibitor, or a monovalent anti-viral agent; wherein the compound is not

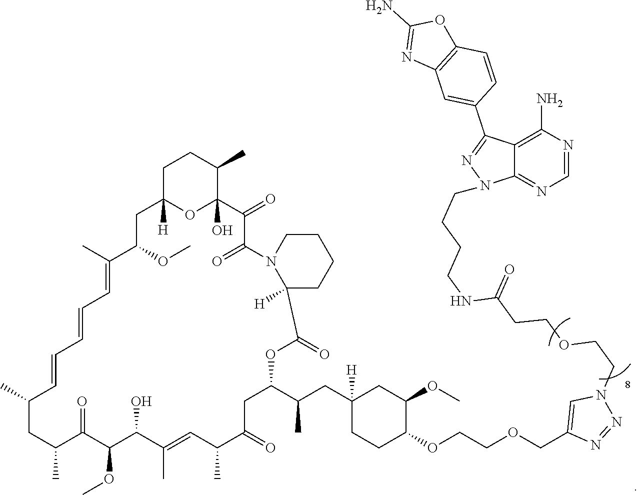

Embodiment P2. The compound of embodiment P1, wherein the immunophilin-binding moiety is a cyclophilin-binding moiety or an FKBP-binding moiety.

Embodiment P3. The compound of one of embodiments P1 to P2, wherein the immunophilin-binding moiety is

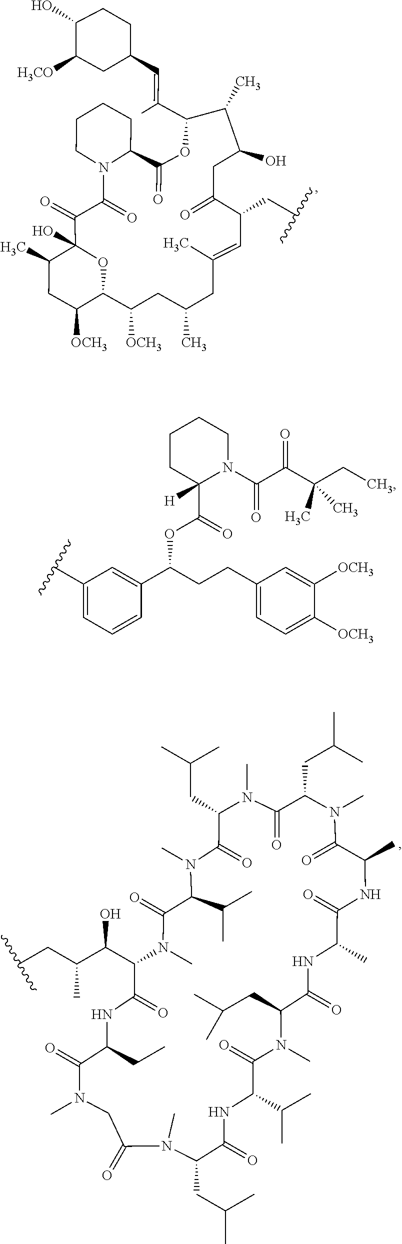

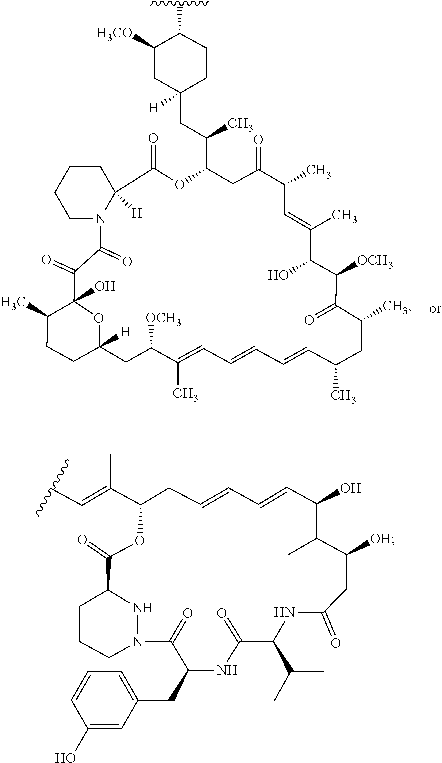 [SMILES: *[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC ; */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

or an analog thereof.

Embodiment P4. The compound of one of embodiments P1 to P2, wherein the immunophilin-binding moiety is

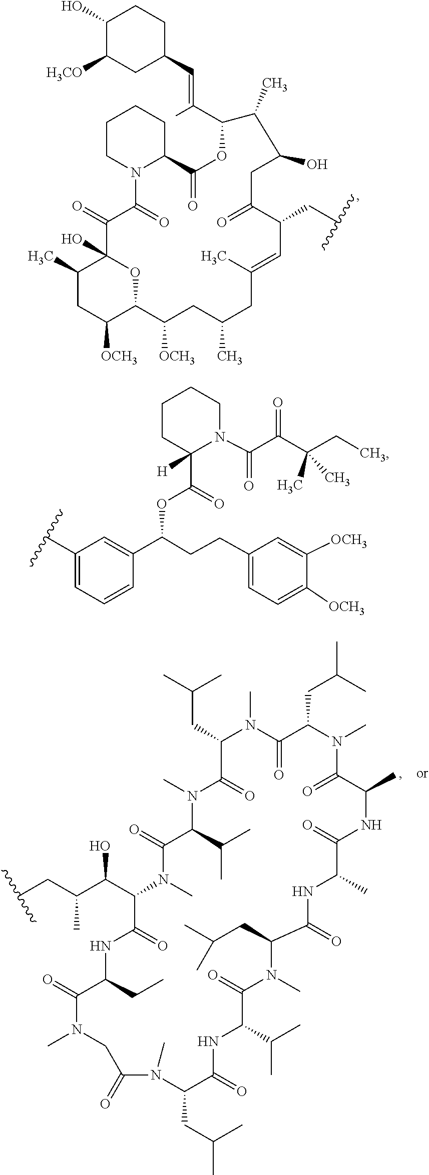

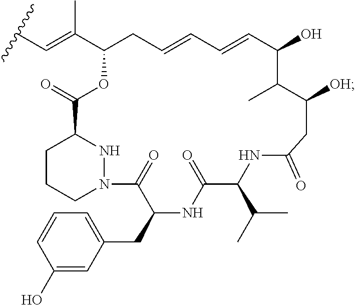 [SMILES: */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

or an analog thereof.

Embodiment P5. The compound of one of embodiments P1 to P3 wherein L¹ is L²-L³-L⁴-L⁵-L⁶;

L² is connected directly to the moiety of an immunophilin-binding compound;
L² is —S(O)₂—, —N(R²)—, —O—, —S—, —C(O)—, —C(O)N(R²)—, —N(R²)C(O)—,
—N(R²)C(O)NH—, —NHC(O)N(R²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

### L⁵ is a

bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

### L⁶ is a

bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R², R³, R⁴, R⁵, and R⁶ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC I₃,
—OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P6. The compound of embodiment P5, wherein L³, L⁴, L⁵, and L⁶ are a bond.

Embodiment P7. The compound of embodiment P5, wherein L² is a substituted or unsubstituted alkylene, substituted or unsubstituted heteroalkylene, substituted or unsubstituted cycloalkylene, or substituted or unsubstituted heterocycloalkylene;

L³ is a bond, substituted or unsubstituted alkylene, substituted or unsubstituted heteroalkylene, substituted or unsubstituted cycloalkylene, or substituted or unsubstituted heterocycloalkylene;
L⁴ is a bond, substituted or unsubstituted alkylene, or substituted or unsubstituted heteroalkylene;
L⁵ is a bond; and
L⁶ is a bond.

Embodiment P8. The compound of embodiment P5, wherein L² is an unsubstituted C₃-C₇ alkylene, an oxo-substituted C₃-C₇ alkylene, an unsubstituted 3 to 17 membered heteroalkylene, or an oxo-substituted 3 to 17 membered heteroalkylene;

L³ is a bond, an unsubstituted C₃-C₇ alkylene, an oxo-substituted C₃-C₇ alkylene, an unsubstituted 3 to 17 membered heteroalkylene, an oxo-substituted 3 to 17 membered heteroalkylene, or an unsubstituted 5 to 6 membered heterocycloalkylene, and
L⁴ is a bond, an unsubstituted C₃-C₇ alkylene, an oxo-substituted C₃-C₇ alkylene, an unsubstituted 3 to 17 membered heteroalkylene, or an oxo-substituted 3 to 17 membered heteroalkylene;
L⁵ is a bond; and
L⁶ is a bond.

Embodiment P9. The compound of one of embodiments P1 to P3, wherein L¹ is a bond, an unsubstituted C₃-C₇ alkylene, an oxo-substituted C₃-C₇ alkylene, an unsubstituted 3 to 17 membered heteroalkylene, or an oxo-substituted 3 to 17 membered heteroalkylene.

Embodiment P10. The compound of one of embodiments P1 to P3, wherein L¹ is

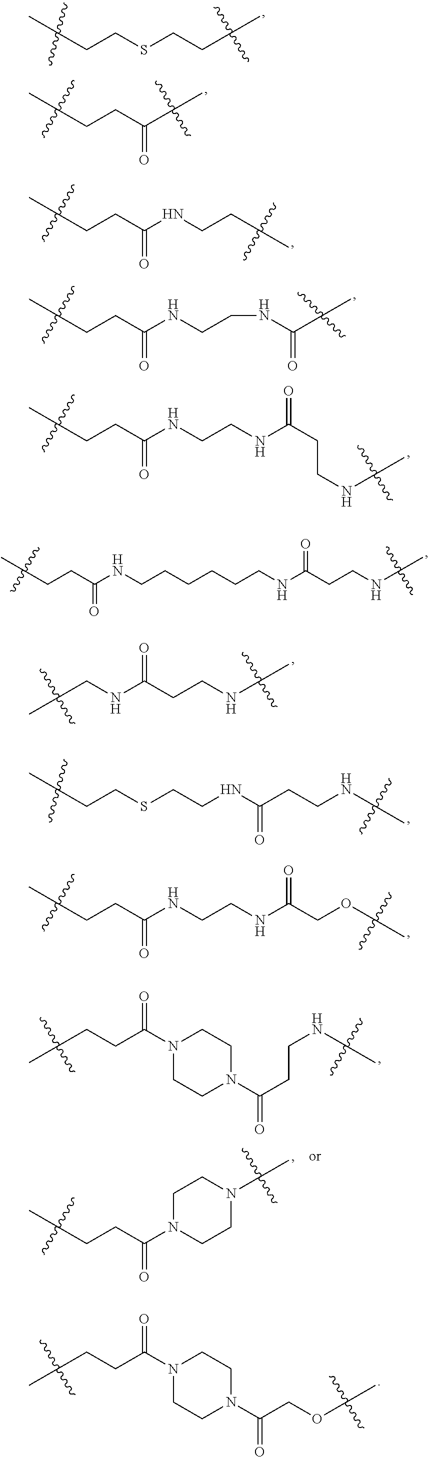

Embodiment P11. The compound of one of embodiments P1 to P3, wherein L¹ is a bond.

Embodiment P12. The compound of one of embodiments P1 to P3, wherein L¹ is a substituted or unsubstituted alkylene or substituted or unsubstituted heteroalkylene.

Embodiment P13. The compound of one of embodiments P1 to P12, wherein R¹ is a monovalent kinase inhibitor.

Embodiment P14. The compound of embodiment P13, wherein the kinase is not mTOR.

Embodiment P15. The compound of one of embodiments P13 to P14, wherein the monovalent kinase inhibitor is a monovalent Src kinase inhibitor.

Embodiment P16. The compound of embodiments P13 to P14, wherein the monovalent Src kinase inhibitor is a monovalent dasatinib or monovalent dasatinib derivative.

Embodiment P17. The compound of embodiment P16, wherein the monovalent dasatinib derivative has the formula:

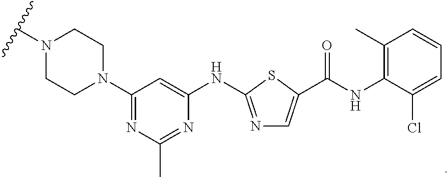 [SMILES: *N1CCN(c2cc(Nc3ncc(C(=O)Nc4c(C)cccc4Cl)s3)nc(C)n2)CC1]

Embodiment P18. The compound of one of embodiments P13 to P14, wherein the monovalent kinase inhibitor is a monovalent Raf inhibitor, VEGFR inhibitor, PDGFR inhibitor, or c-Kit inhibitor.

Embodiment P19. The compound of embodiment P18, wherein the monovalent Raf inhibitor, VEGFR inhibitor, PDGFR inhibitor, or c-Kit inhibitor is a monovalent sorafenib or monovalent sorafenib derivative.

Embodiment P20. The compound of embodiment P19, wherein the monovalent sorafenib derivative has the formula:

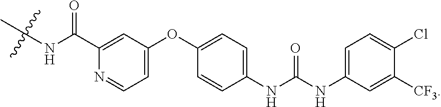 [SMILES: *NC(=O)c1cc(Oc2ccc(NC(=O)Nc3ccc(Cl)c(C(F)(F)F)c3)cc2)ccn1]

Embodiment P21. The compound of one of embodiments P13 to P14, wherein the monovalent kinase inhibitor is a monovalent EGFR inhibitor.

Embodiment P22. The compound of embodiment P21, wherein the monovalent EGFR inhibitor is a monovalent lapatinib, monovalent lapatinib derivative, monovalent erlotinib, monovalent erlotinib derivative, monovalent gefitinib, or monovalent gefitinib derivative.

Embodiment P23. The compound of embodiment P22, wherein the monovalent EGFR inhibitor has the formula:

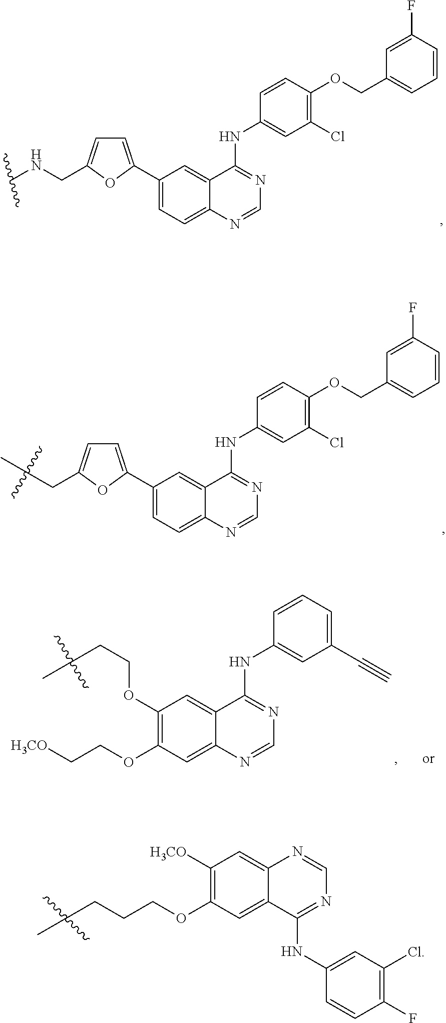 [SMILES: *NCc1ccc(-c2ccc3ncnc(Nc4ccc(OCc5cccc(F)c5)c(Cl)c4)c3c2)o1 ; *Cc1ccc(-c2ccc3ncnc(Nc4ccc(OCc5cccc(F)c5)c(Cl)c4)c3c2)o1 ; *CCOc1cc2c(Nc3cccc(C#C)c3)ncnc2cc1OCCOC ; *CCCOc1cc2c(Nc3ccc(F)c(Cl)c3)ncnc2cc1OC]

Embodiment P24. The compound of one of embodiments P13 to P14, wherein the monovalent kinase inhibitor is a monovalent LRRK2 inhibitor.

Embodiment P25. The compound of embodiment P24, wherein the monovalent LRRK2 inhibitor is a monovalent GNE-7915 or monovalent GNE-7915 derivative.

Embodiment P26. The compound of embodiment P25, wherein the monovalent GNE-7915 derivative has the formula:

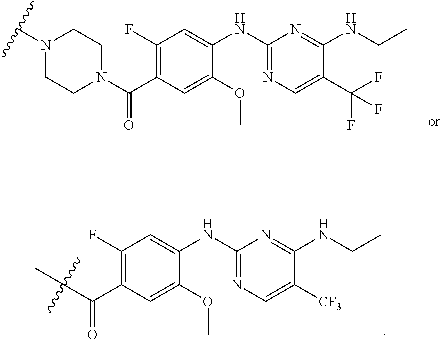 [SMILES: *N1CCN(C(=O)c2cc(OC)c(Nc3ncc(C(F)(F)F)c(NCC)n3)cc2F)CC1 ; *C(=O)c1cc(OC)c(Nc2ncc(C(F)(F)F)c(NCC)n2)cc1F]

Embodiment P27. The compound of one of embodiments P1 to P12, wherein R¹ is a monovalent KRAS inhibitor.

Embodiment P28. The compound of embodiment P27, wherein the monovalent KRAS inhibitor is a monovalent KRAS G12C inhibitor or a monovalent KRAS M72C inhibitor.

Embodiment P29. The compound of embodiment P28, wherein the monovalent KRAS inhibitor has the formula:

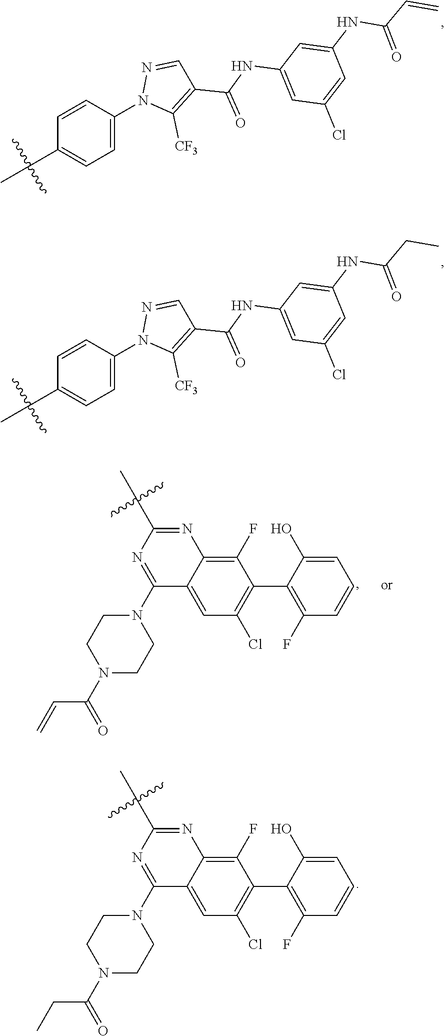

Embodiment P30. The compound of one of embodiments P1 to P29, wherein the compound is not a calcineurin inhibitor.

Embodiment P31. A pharmaceutical composition comprising a pharmaceutically acceptable excipient and a compound of one of embodiments P1 to P29.

Embodiment P32. A method of treating a disease associated with aberrant enzyme activity in a subject in need of such treatment, comprising administering a compound of one of embodiments 1 to 29 to the subject.

Embodiment P33. The method of embodiment P32, wherein the enzyme activity is a kinase activity.

Embodiment P34. The method of embodiment P33, wherein the kinase activity is in the CNS of the subject.

Embodiment P35. A method of treating a disease in a subject in need of such treatment, comprising administering a compound of one of embodiments P1 to P29 to the subject, wherein the disease is a viral disease, cancer, or a neurodegenerative disease.

Embodiment P36. The method of embodiment P35, wherein the disease is cancer.

Embodiment P37. The method of embodiment P36, wherein the cancer is glioblastoma or glioma.

Embodiment P38. The method of embodiment P35, wherein the disease is a neurodegenerative disease.

Embodiment P39. The method of embodiment P38, wherein the neurodegenerative disease is Parkinson's Disease.

### VI. Additional Embodiments

Embodiment 1. A compound having the formula:

A-L¹-R¹;

wherein
A is an immunophilin-binding moiety;
L¹ is a bond or a covalent linker; and
R¹ is a kinase inhibitor, a pseudokinase inhibitor, a GTPase inhibitor, a histone-modifying enzyme inhibitor, or a monovalent anti-viral agent; wherein the compound is not

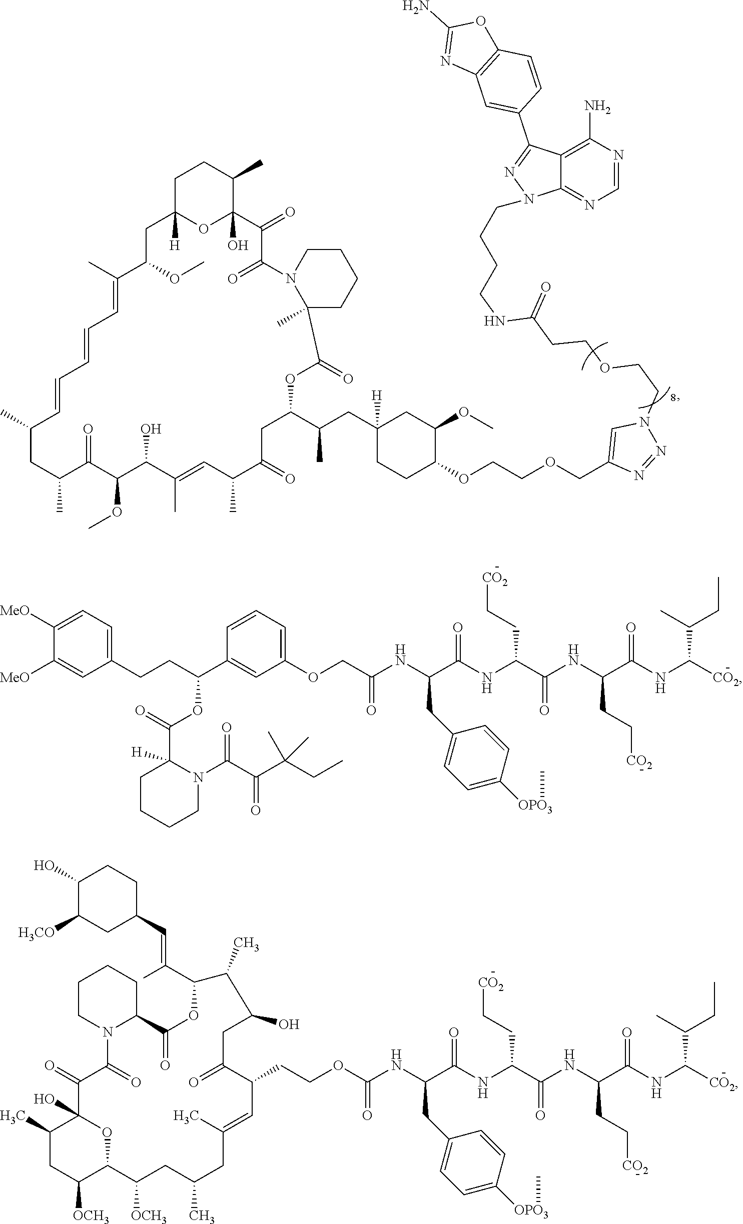

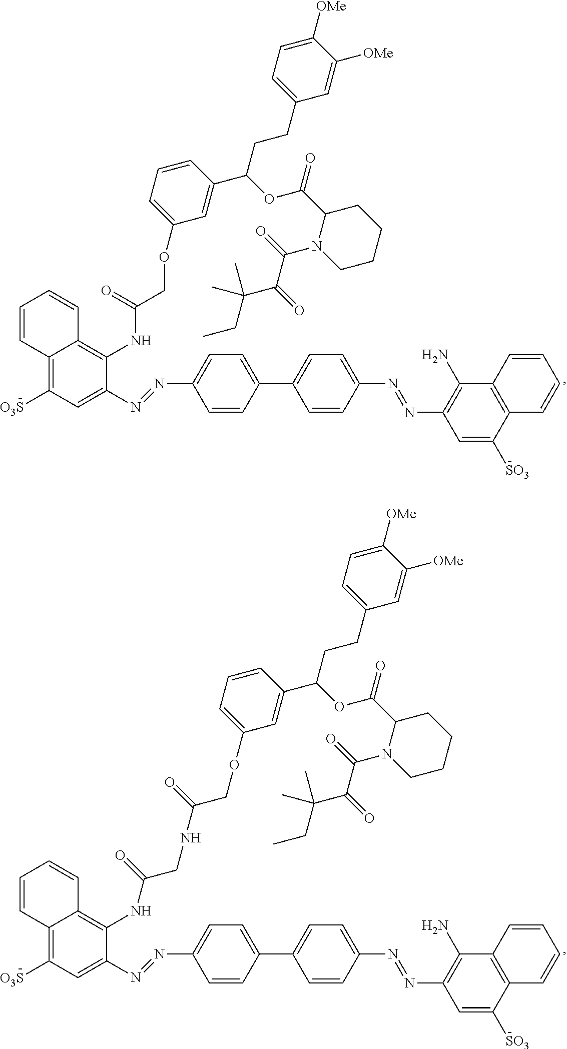

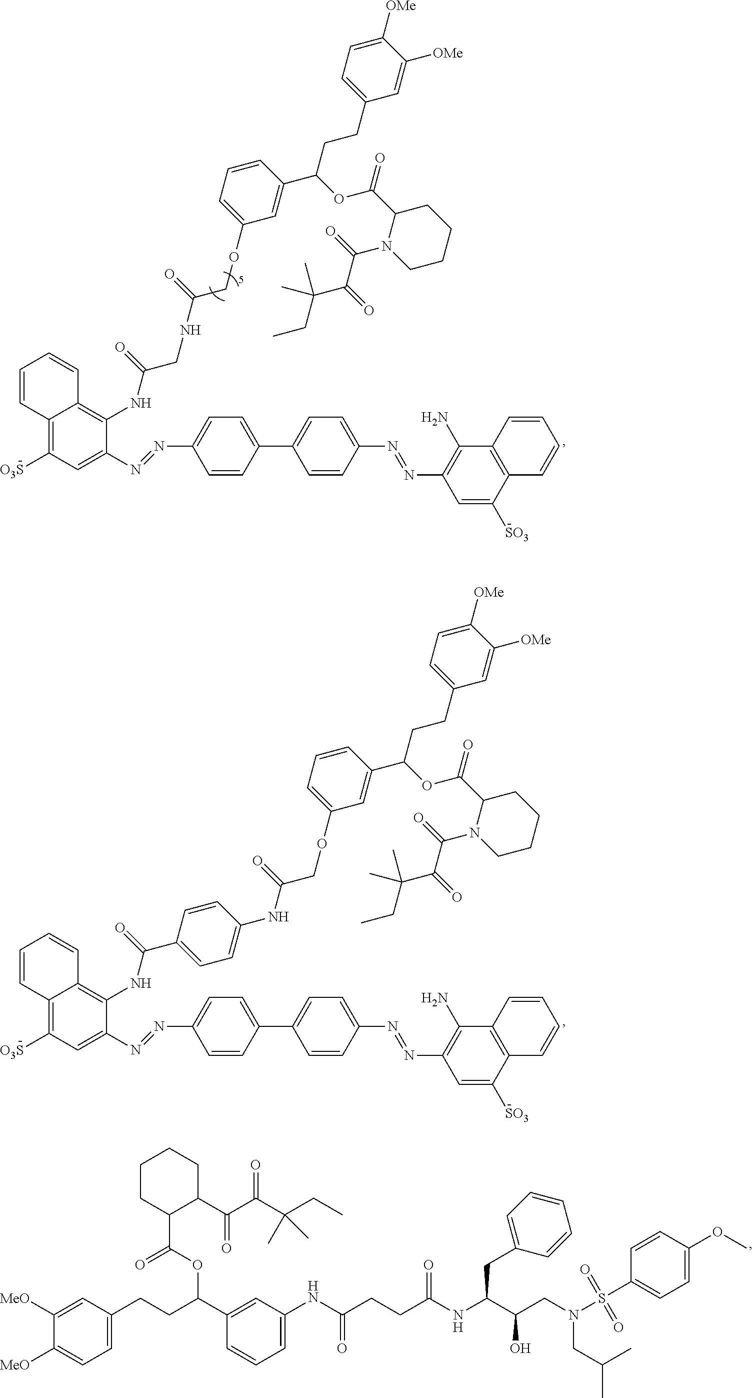

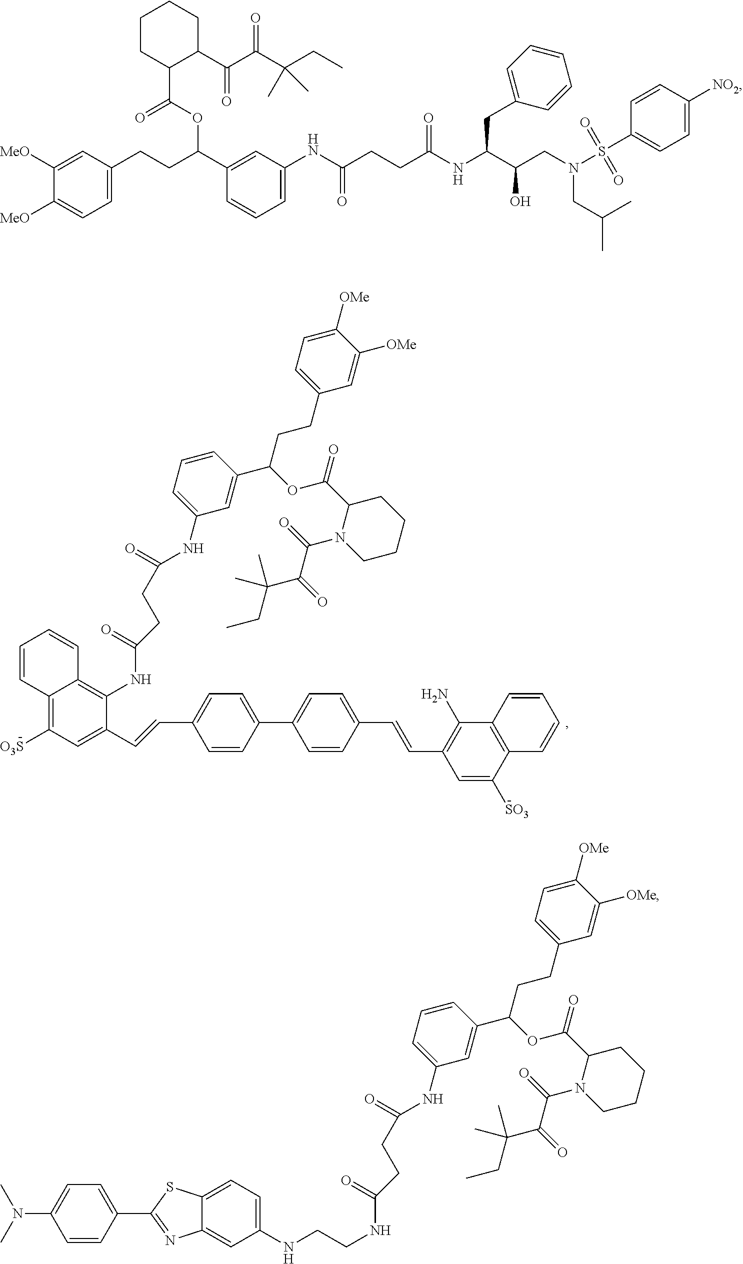

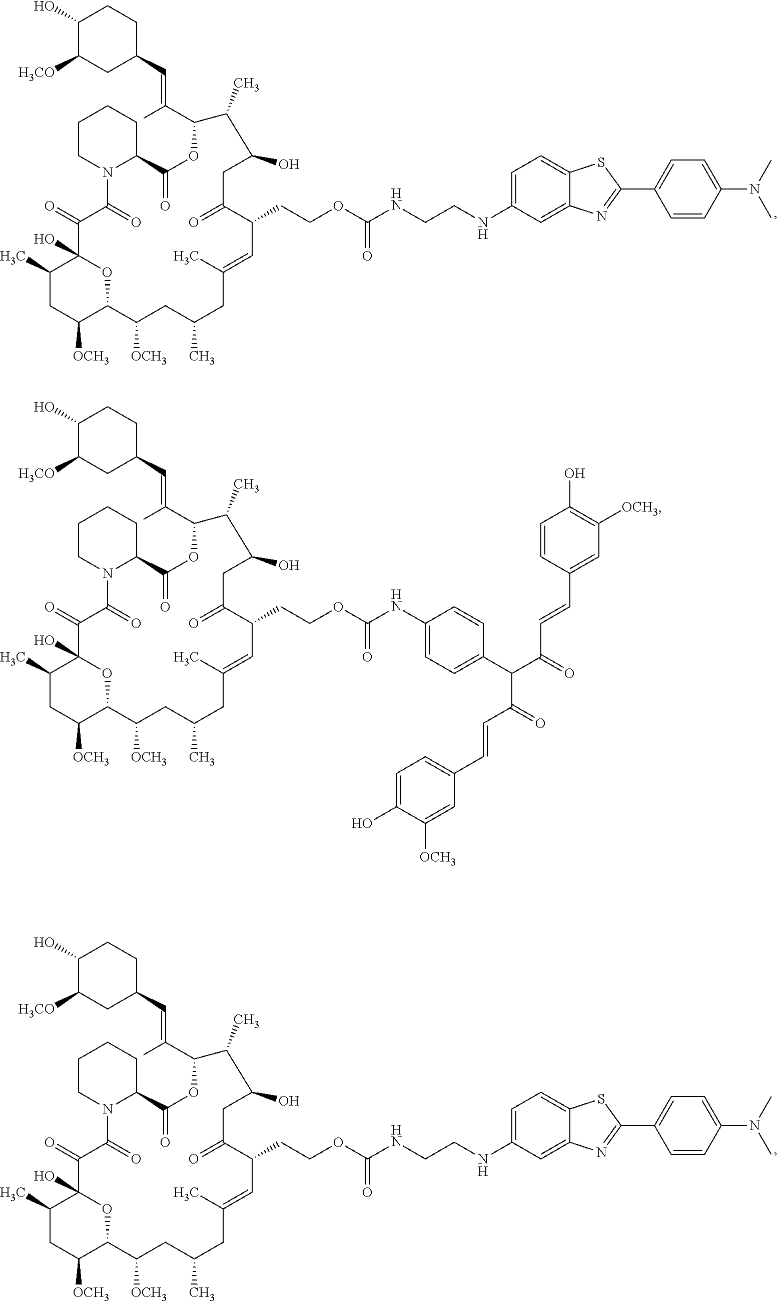

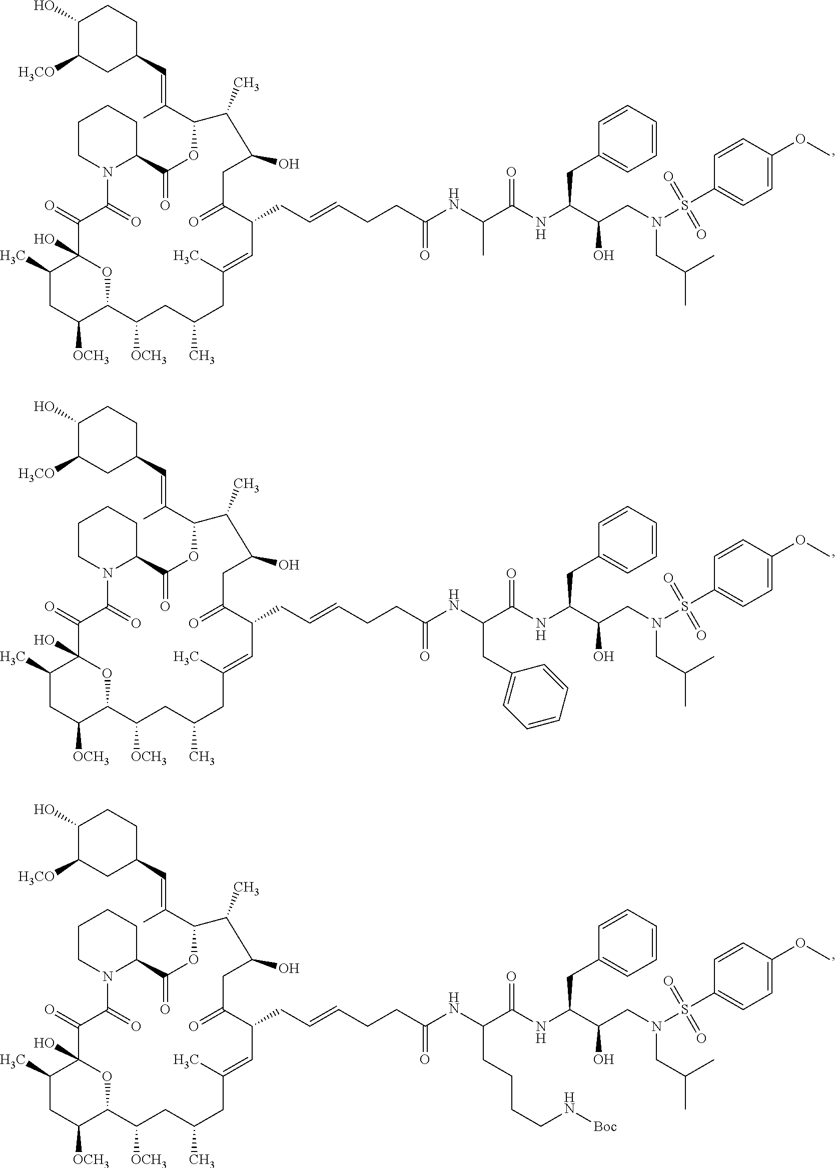

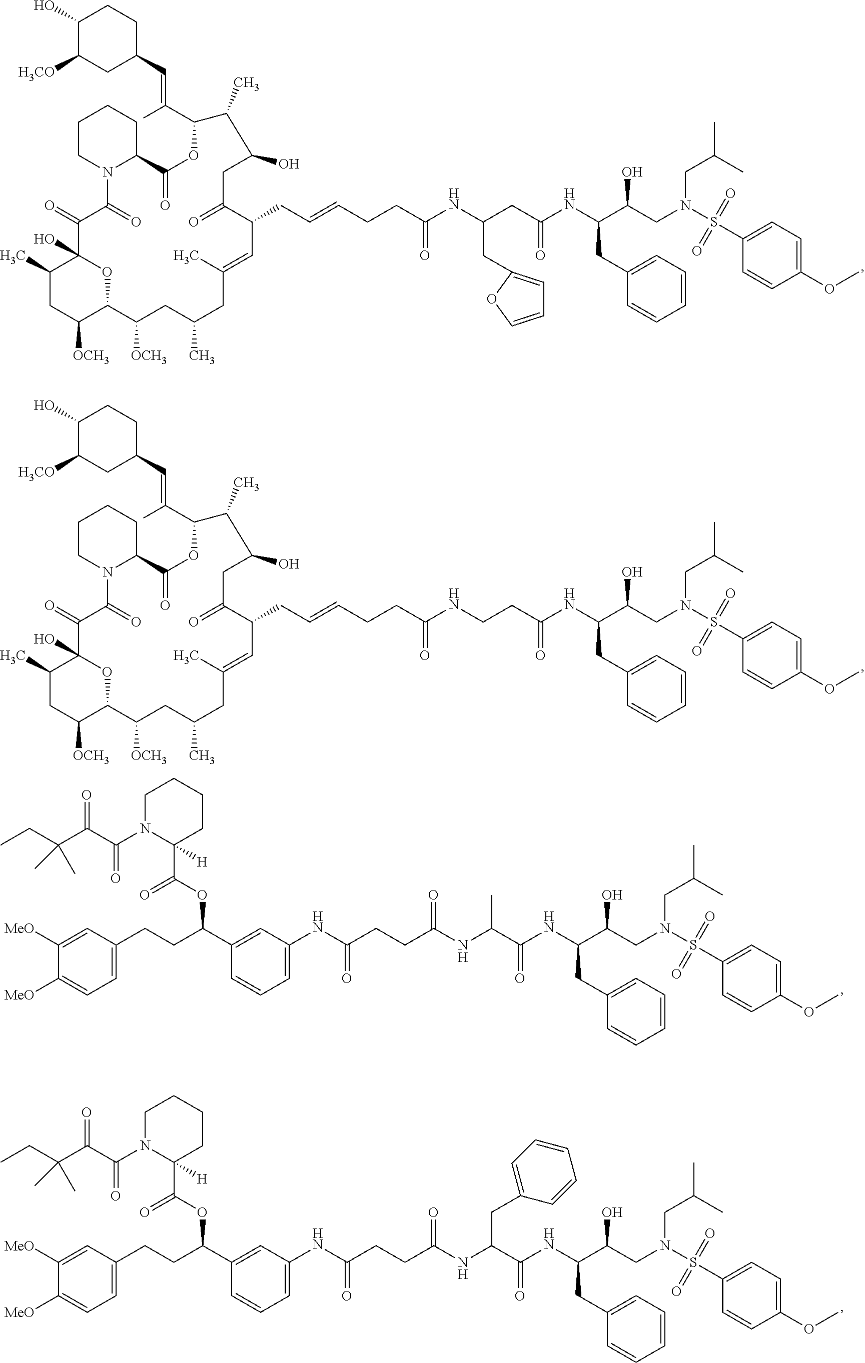

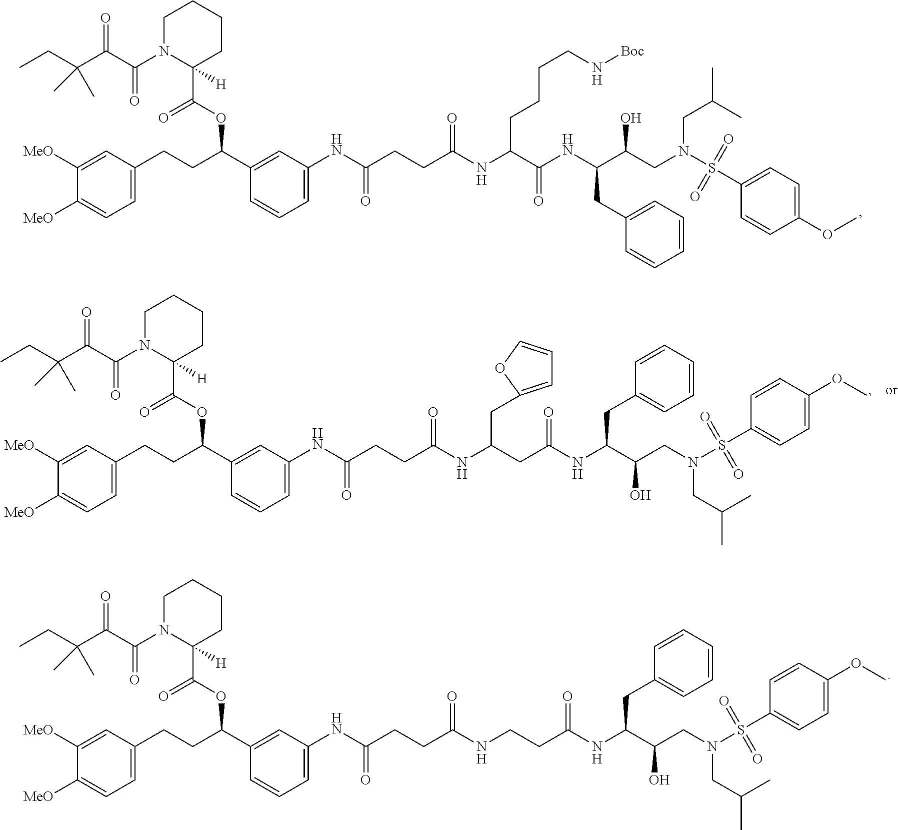

Embodiment 2. The compound of embodiment 1, wherein R¹ is not a monovalent human immunodeficiency (HIV) protease inhibitor or an amyloid β aggregation inhibitor.

Embodiment 3. The compound of embodiment 1, wherein the immunophilin-binding moiety is a cyclophilin-binding moiety or an FKBP-binding moiety.

Embodiment 4. The compound of one of embodiments 1 to 3, wherein the immunophilin-binding moiety is

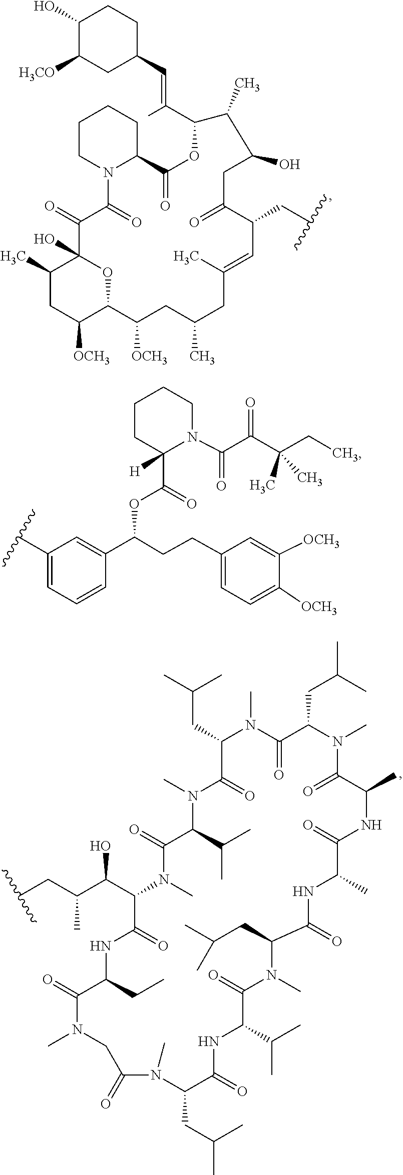

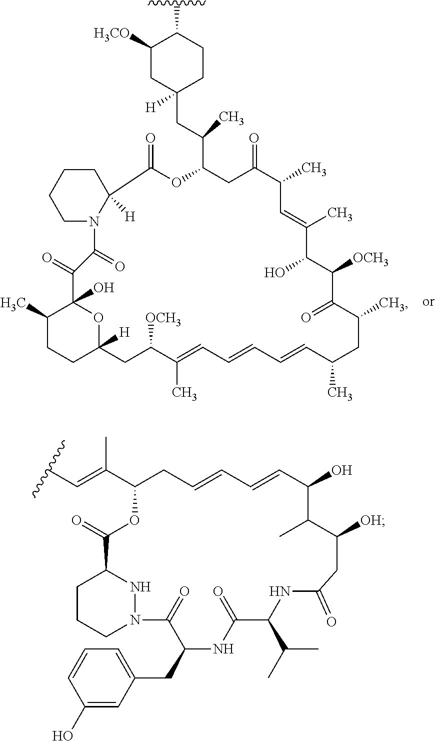 [SMILES: *[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC ; "or" ; */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

or an analog thereof.

Embodiment 5. The compound of one of embodiments 1 to 3, wherein the immunophilin-binding moiety is

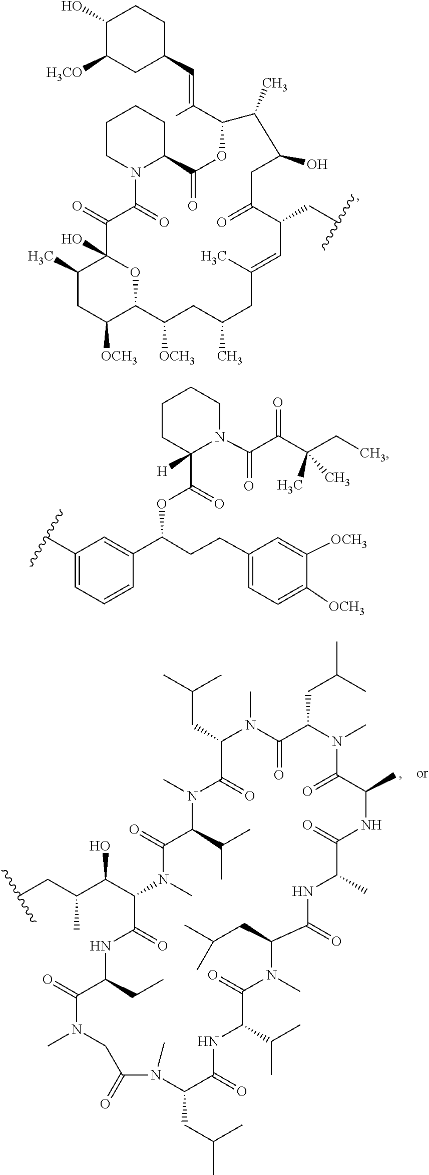

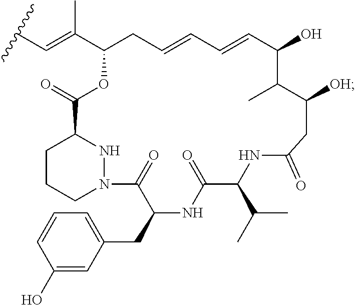 [SMILES: */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

or an analog thereof.

Embodiment 6. The compound of one of embodiments 1 to 4 wherein L¹ is L²-L³-L⁴-L⁵-L⁶;

L² is connected directly to the moiety of an immunophilin-binding compound;
L² is a bond, —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

### L⁵ is a

bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

### L⁶ is a

bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R², R³, R⁴, R⁵, and R⁶ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC I₃,
—OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 7. The compound of embodiment 6, wherein L³, L⁴, L⁵, and L⁶ are a bond.

Embodiment 8. The compound of embodiment 6, wherein L² is a substituted or unsubstituted alkylene, substituted or unsubstituted heteroalkylene, substituted or unsubstituted cycloalkylene, or substituted or unsubstituted heterocycloalkylene;

L³ is a bond, substituted or unsubstituted alkylene, substituted or unsubstituted heteroalkylene, substituted or unsubstituted cycloalkylene, or substituted or unsubstituted heterocycloalkylene;
L⁴ is a bond, substituted or unsubstituted alkylene, or substituted or unsubstituted heteroalkylene;
L⁵ is a bond; and
L⁶ is a bond.

Embodiment 9. The compound of embodiment 6, wherein L² is an unsubstituted C₃-C₇ alkylene, an oxo-substituted C₃-C₇ alkylene, an unsubstituted 3 to 17 membered heteroalkylene, or an oxo-substituted 3 to 17 membered heteroalkylene;

L³ is a bond, an unsubstituted C₃-C₇ alkylene, an oxo-substituted C₃-C₇ alkylene, an unsubstituted 3 to 17 membered heteroalkylene, an oxo-substituted 3 to 17 membered heteroalkylene, or an unsubstituted 5 to 6 membered heterocycloalkylene, and L⁴ is a bond, an unsubstituted C₃-C₇ alkylene, an oxo-substituted C₃-C₇ alkylene, an unsubstituted 3 to 17 membered heteroalkylene, or an oxo-substituted 3 to 17 membered heteroalkylene;
L⁵ is a bond; and
L⁶ is a bond.

Embodiment 10. The compound of one of embodiments 1 to 4, wherein L¹ is a bond, an unsubstituted C₃-C₇ alkylene, an oxo-substituted C₃-C₇ alkylene, an unsubstituted 3 to 17 membered heteroalkylene, or an oxo-substituted 3 to 17 membered heteroalkylene.

Embodiment 11. The compound of one of embodiments 1 to 4, wherein L¹ is

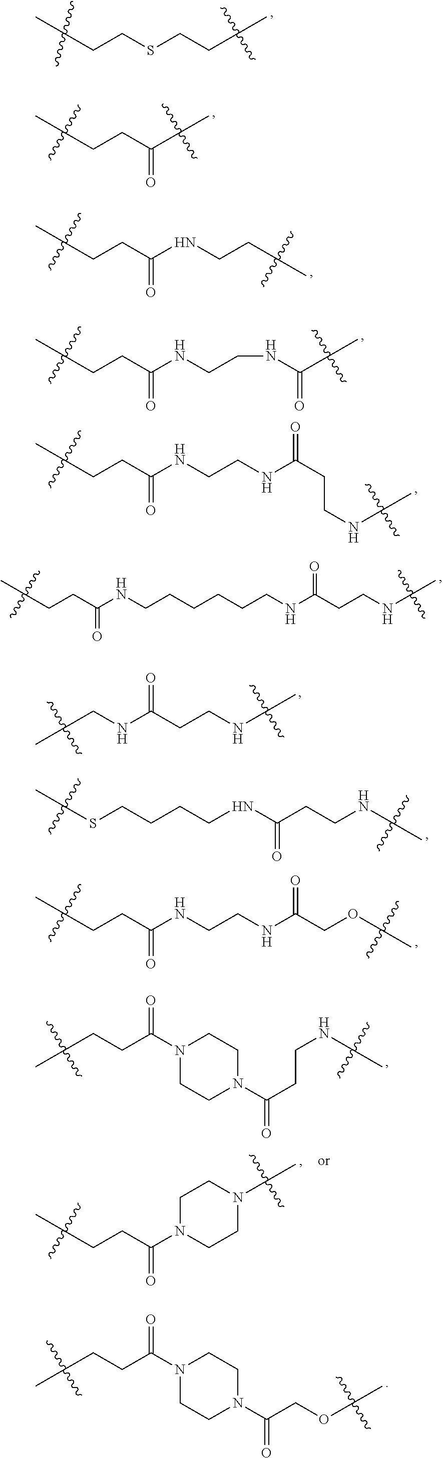

Embodiment 12. The compound of one of embodiments 1 to 4, wherein L¹ is a bond.

Embodiment 13. The compound of one of embodiments 1 to 4, wherein L¹ is a substituted or unsubstituted alkylene or substituted or unsubstituted heteroalkylene.

Embodiment 14. The compound of one of embodiments 1 to 13, wherein R¹ is a monovalent kinase inhibitor.

Embodiment 15. The compound of embodiment 14, wherein the kinase is not mTOR.

Embodiment 16. The compound of one of embodiments 14 to 15, wherein the monovalent kinase inhibitor is a monovalent Src kinase inhibitor.

Embodiment 17. The compound of one of embodiments 14 to 15, wherein the monovalent Src kinase inhibitor is a monovalent dasatinib or monovalent dasatinib derivative.

Embodiment 18. The compound of embodiment 17, wherein the monovalent dasatinib derivative has the formula:

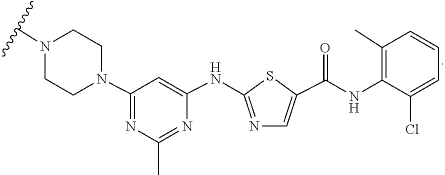 [SMILES: *N1CCN(c2cc(Nc3ncc(C(=O)Nc4c(C)cccc4Cl)s3)nc(C)n2)CC1]

Embodiment 19. The compound of one of embodiments 14 to 15, wherein the monovalent kinase inhibitor is a monovalent Raf inhibitor, VEGFR inhibitor, PDGFR inhibitor, or c-Kit inhibitor.

Embodiment 20. The compound of embodiment 19, wherein the monovalent Raf inhibitor, VEGFR inhibitor, PDGFR inhibitor, or c-Kit inhibitor is a monovalent sorafenib or monovalent sorafenib derivative.

Embodiment 21. The compound of embodiment 20, wherein the monovalent sorafenib derivative has the formula:

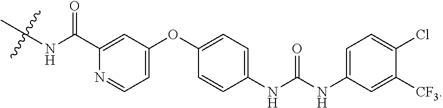 [SMILES: *NC(=O)c1cc(Oc2ccc(NC(=O)Nc3ccc(Cl)c(C(F)(F)F)c3)cc2)ccn1]

Embodiment 22. The compound of one of embodiments 14 to 15, wherein the monovalent kinase inhibitor is a monovalent EGFR inhibitor.

Embodiment 23. The compound of embodiment 22, wherein the monovalent EGFR inhibitor is a monovalent lapatinib, monovalent lapatinib derivative, monovalent erlotinib, monovalent erlotinib derivative, monovalent gefitinib, or monovalent gefitinib derivative.

Embodiment 24. The compound of embodiment 23, wherein the monovalent EGFR inhibitor has the formula:

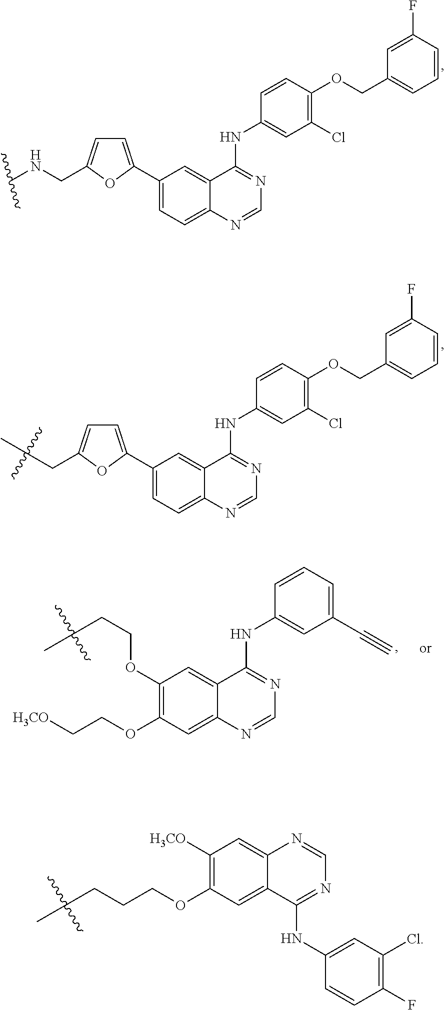 [SMILES: *NCc1ccc(-c2ccc3ncnc(Nc4ccc(OCc5cccc(F)c5)c(Cl)c4)c3c2)o1 ; *Cc1ccc(-c2ccc3ncnc(Nc4ccc(OCc5cccc(F)c5)c(Cl)c4)c3c2)o1 ; *CCOc1cc2c(Nc3cccc(C#C)c3)ncnc2cc1OCCOC ; *CCCOc1cc2c(Nc3ccc(F)c(Cl)c3)ncnc2cc1OC]

Embodiment 25. The compound of one of embodiments 14 to 15, wherein the monovalent kinase inhibitor is a monovalent LRRK2 inhibitor.

Embodiment 26. The compound of embodiment 25, wherein the monovalent LRRK2 inhibitor is a monovalent GNE-7915 or monovalent GNE-7915 derivative.

Embodiment 27. The compound of embodiment 26, wherein the monovalent GNE-7915 derivative has the formula:

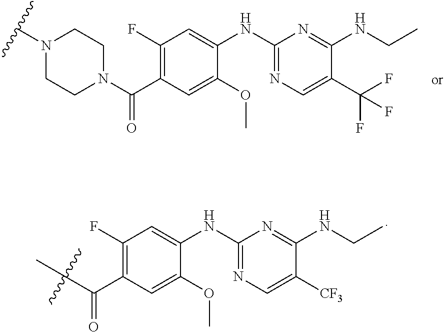 [SMILES: *N1CCN(C(=O)c2cc(OC)c(Nc3ncc(C(F)(F)F)c(NCC)n3)cc2F)CC1 ; *C(=O)c1cc(OC)c(Nc2ncc(C(F)(F)F)c(NCC)n2)cc1F]

Embodiment 28. The compound of one of embodiments 14 to 15, wherein the monovalent kinase inhibitor is a monovalent MAP4K inhibitor.

Embodiment 29. The compound of embodiment 28, wherein the monovalent MAP4K inhibitor is a monovalent HGK inhibitor.

Embodiment 30. The compound of embodiment 29, wherein the monovalent HGK inhibitor has the formula:

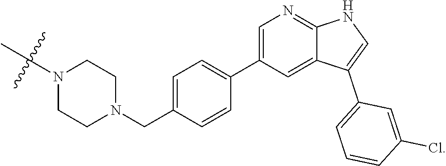 [SMILES: *N1CCN(Cc2ccc(-c3cnc4[nH]cc(-c5cccc(Cl)c5)c4c3)cc2)CC1]

Embodiment 31. The compound of one of embodiments 14 to 15, wherein the monovalent kinase inhibitor is a monovalent MAP3K inhibitor.

Embodiment 32. The compound of embodiment 31, wherein the monovalent MAP3K inhibitor is a monovalent DLK inhibitor.

Embodiment 33. The compound of embodiment 32, wherein the monovalent DLK inhibitor has the formula:

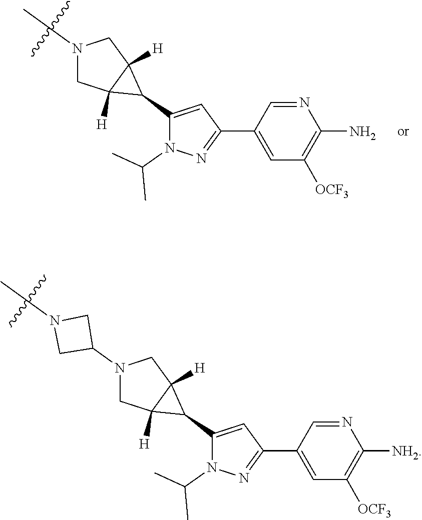 [SMILES: *N1C[C@@H]2[C@H](C1)[C@H]2c1cc(-c2cnc(N)c(OC(F)(F)F)c2)nn1C(C)C ; *N1CC(N2C[C@@H]3[C@H](C2)[C@H]3c2cc(-c3cnc(N)c(OC(F)(F)F)c3)nn2C(C)C)C1]

Embodiment 34. The compound of one of embodiments 1 to 13, wherein R¹ is a monovalent KRAS inhibitor.

Embodiment 35. The compound of embodiment 34, wherein the monovalent KRAS inhibitor is a monovalent KRAS G12C inhibitor or a monovalent KRAS M72C inhibitor.

Embodiment 36. The compound of embodiment 35, wherein the monovalent KRAS inhibitor has the formula:

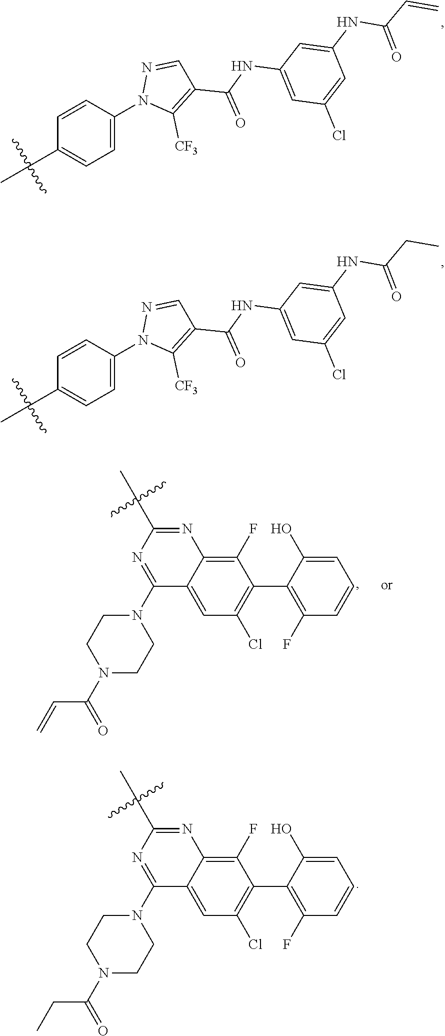

Embodiment 37. The compound of one of embodiments 1 to 36, wherein the compound is not a calcineurin inhibitor.

Embodiment 38. A pharmaceutical composition comprising a pharmaceutically acceptable excipient and a compound of one of embodiments 1 to 36.

Embodiment 39. A method of treating a disease associated with aberrant enzyme activity in a subject in need of such treatment, comprising administering a compound of one of embodiments 1 to 36 to the subject.

Embodiment 40. The method of embodiment 39, wherein the enzyme activity is a kinase activity.

Embodiment 41. The method of embodiment 40, wherein the kinase activity is in the CNS of the subject.

Embodiment 42. A method of treating a disease in a subject in need of such treatment, comprising administering a compound of one of embodiments 1 to 36 to the subject, wherein the disease is a viral disease, cancer, or a neurodegenerative disease.

Embodiment 43. The method of embodiment 42, wherein the disease is cancer.

Embodiment 44. The method of embodiment 43, wherein the cancer is glioblastoma or glioma.

Embodiment 45. The method of embodiment 42, wherein the disease is a neurodegenerative disease.

Embodiment 46. The method of embodiment 45, wherein the neurodegenerative disease is Parkinson's Disease.

Embodiment 47. The method of embodiment 45, wherein the neurodegenerative disease is Amyotrophic lateral sclerosis (ALS).

Embodiment 48. The method of embodiment 45, wherein the neurodegenerative disease is Alzheimer's disease.

### EXAMPLES

Here we describe a method to construct bispecific chemical ligands that induce the association of protein kinases and a ubiquitously expressed protein, FKBP12, by chemically linking a kinase inhibitor to a high affinity ligand of FKBP, FK506. We show that these bispecific ligands are cell-permeable and effect potent, specific and long-lasting inhibition of their respective targets, and that their cellular activity is amenable to modulation with a separate ligand of FKBP12. We exemplify our approach with three case studies: an inhibitor of Src-family kinases based on dasatinib, an inhibitor of EGFR/HER2 based on lapatinib, and an inhibitor of LRRK2 based GNE7915.

### Example 1: Ligand Design

We chose dasatinib, an FDA-approved pan-Src family kinase inhibitor, for our model study as abundant literature on its pharmacological properties and structural information is available to aid our initial design and analysis. Inspection of the crystal structures of FKBP12-FK506 complex (PDB: 1FKJ) and dasatinib-Src complex (PDB: 3G5D) revealed that the allyl group at the C21 position of FK506 and the hydroxylethyl group in dasatinib on the piperazine ring are exposed to solvent and serve as suitable sites for chemical fusing Previous structure-activity relationship studies on FK506 and dasatinib also indicate that chemical alterations at these two sites have minimal impact on their affinities for their respective targets. We envisioned that modifying the C21 allyl group confers an additional advantage: substituents larger than allyl at this position will ablate FK506's ability to inhibit its natural target calcineurin, an undesirable activity in our present application. To synthesize the bispecific ligand FK506-Dasatinib, we employed HATU-mediated amide coupling reaction to join a carboxylic acid derived from FK506 and a secondary amine derived from Dasatinib. The synthetic route used is amenable to incorporating linkers with various length and geometry for further optimization.

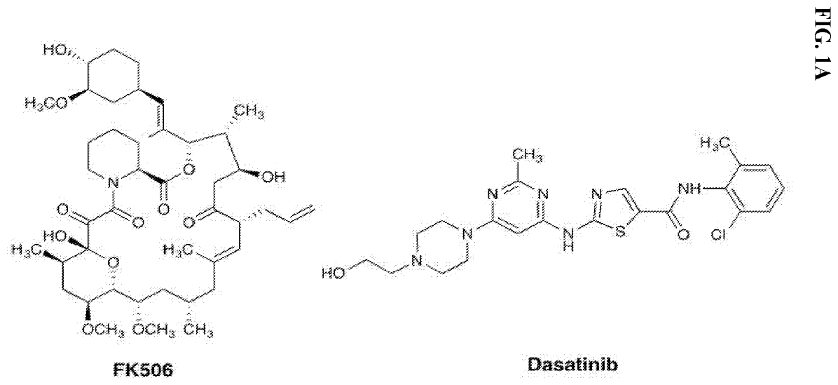

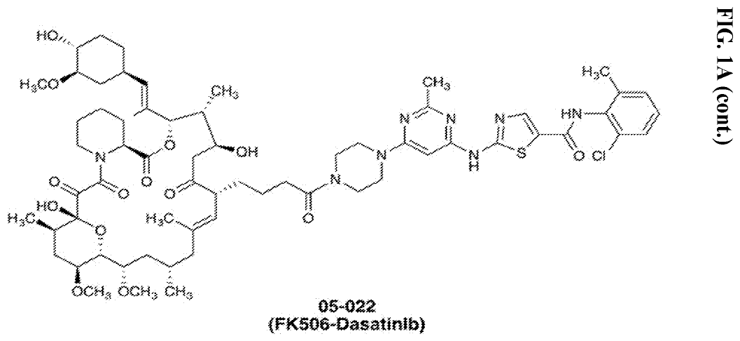

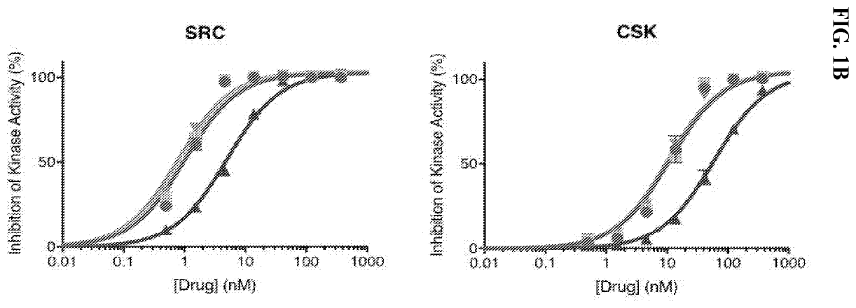

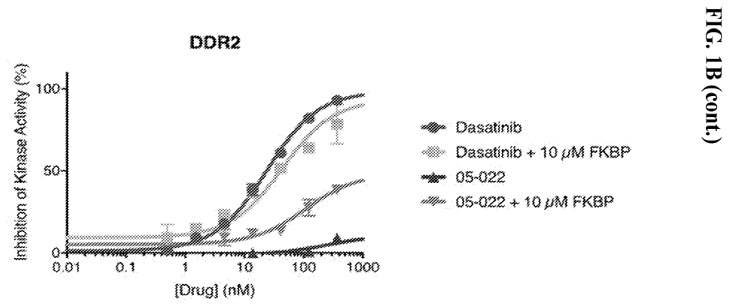

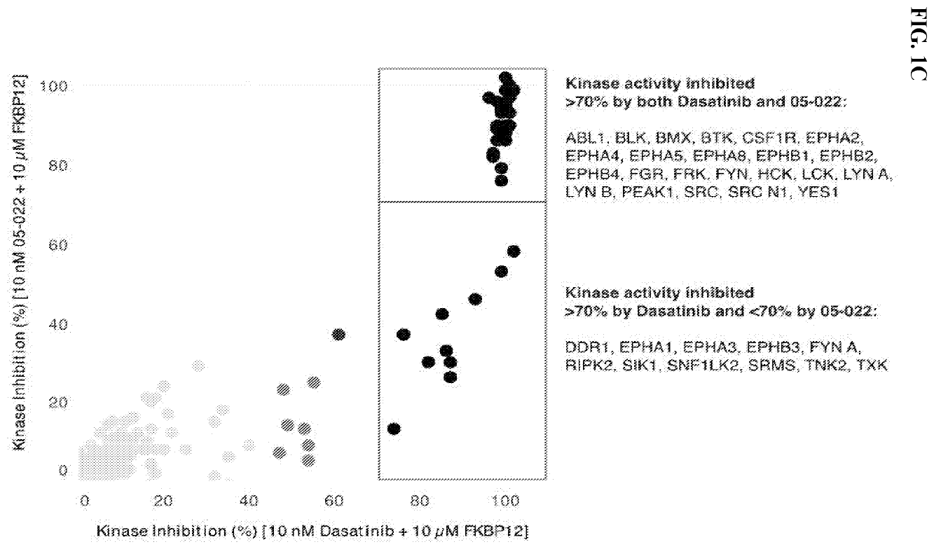

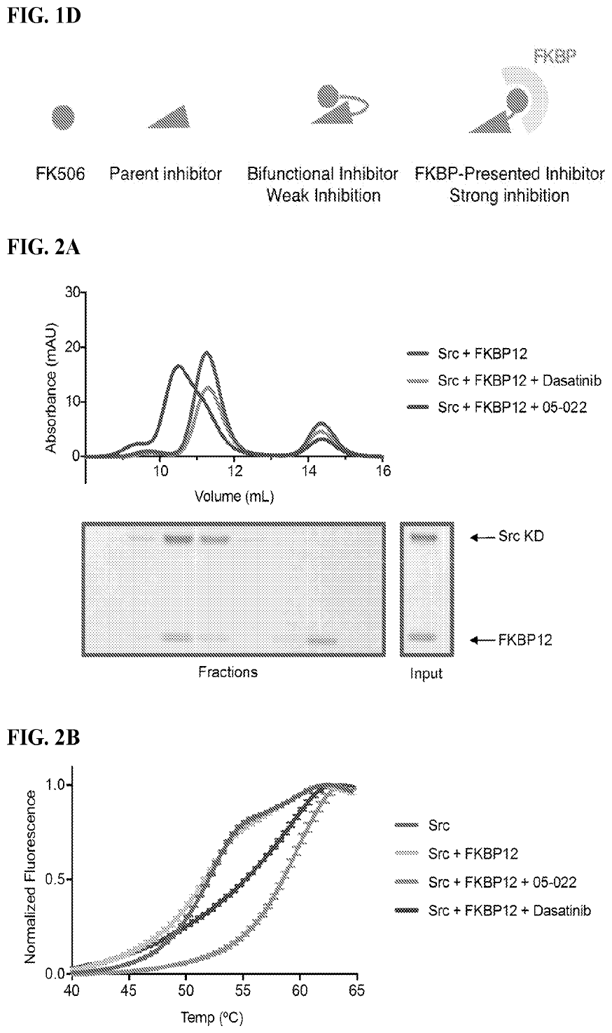

Using a fluorescence polarization assay, we found that FK506-Dasatinib maintained potent binding to FKBP12 (Kd=23 nM), consistent with our previous anticipation. To assess the kinase inhibition activity of FK-Dasatinib, we performed in vitro kinase assays with ATP concentrations at the apparent Km values of each kinase. Three kinases were chosen in this preliminary investigation: Src, Csk and DDR2. Src and Csk are both Src-family tyrosine kinases but with opposite functions in cellular signal transduction, while DDR2 is a receptor tyrosine kinase also potently inhibited by dasatinib. Under standard assay conditions, FK-Dasatinib showed weaker inhibitory activity toward all three kinases compared to dasatinib, with IC₅₀ values more than ten-fold greater those of the latter (FIG. 1). To further mimic the cellular environment, we supplemented the assay buffer with 10 μM recombinant FKBP12, a concentration chosen to match the estimated intracellular concentration of FKBP proteins. At this FKBP concentration, we also ensured that >99.7% of the FK-Dasatinib population would be in complex with FKBP12. Under the new assay conditions, we observed a significant left-shift of the inhibition curves for FK-Dasatinib, whereas the potency of dasatinib remained unchanged. For Src and Csk, the two inhibitors achieved equipotent inhibition upon FKBP12 supplementation. Meanwhile, for DDR2, though enhancement of activity of FK-Dasatinib was also observed, it was still inferior to dasatinib, failing to fully inhibit this kinase even at 1 μM concentration. This difference prompted us to investigate if linking FK506 to dasatinib had reshaped its selectivity for kinase targets. We profiled these two inhibitors against a panel of 485 protein kinases at 10 nM inhibitor concentration and with 10 μM supplemented FKBP12 protein (FIG. 1C). Of these 485 kinases, 23 were inhibited >70% by both inhibitors, and another 11 were inhibited >70% by dasatinib but not FK-Dasatinib. Overall, FK-Dasatinib did not achieve greater inhibition of any kinase tested than dasatinib at 10 nM, but certain kinases (for example, DDR1) appeared to be more disfavored by FK-Dasatinib than others. This differential attenuation of inhibitory activity may be attributed to the favorable or unfavorable interactions with FKBP12 that the kinase must experience in order to bind the FK-Dasatinib/FKBP12 complex. In this model, we envision that when FKBP12 binds FK-dasatinib, a composite surface is formed that presents the dasatinib moiety and surveys various proteins for energetically favorable binding events (FIG. 1D).

### Example 2: Complex Formation

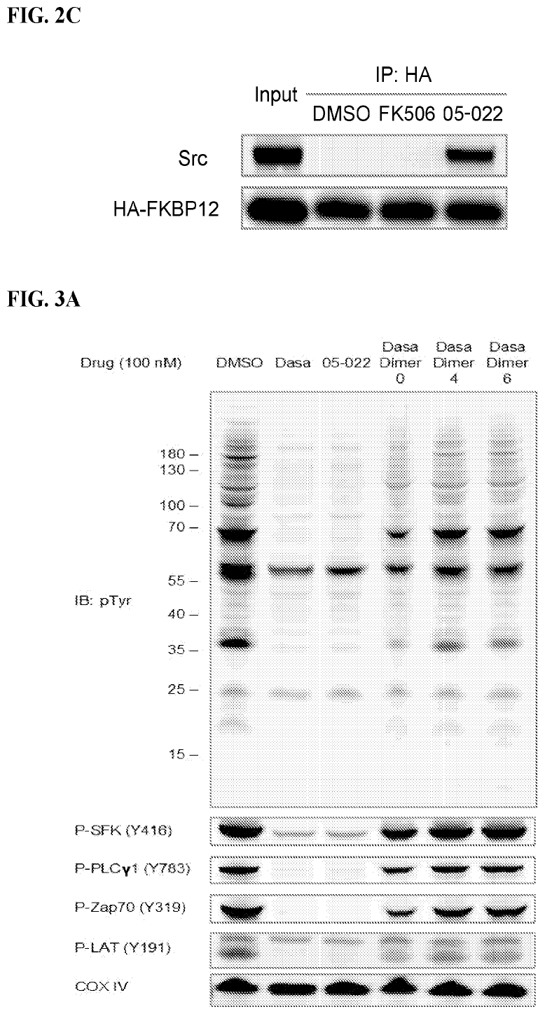

The participation of FKBP12 in the inhibition of kinases by FK-dasatinib is further revealed by its ligand-dependent association with kinases. Addition of FK-Dasatinib to a mixture of recombinant Src kinase domain (33 kDa) and FKBP12 (12 kDa) induced the formation of a stable complex (˜50 kDa) that can be purified by size exclusion chromatography (FIG. 2A). The molecular weight of the complex suggests a 1:1:1 stoichiometry consistent with the anticipated binding mechanism of FK-dasatinib. Differential scanning fluorimetry suggested that formation of this complex led to stabilization of both protein components toward thermal denaturation to a greater extent than dasatinib alone (FIG. 2B). Such tripartite interactions are preserved in more complex native environments—Src co-immunoprecipitated with HA-FKBP12 in Jurkat cell lysates treated with FK-Dasatinib, but not FK506 (FIG. 2C).

### Example 3: Evaluating Efficacy

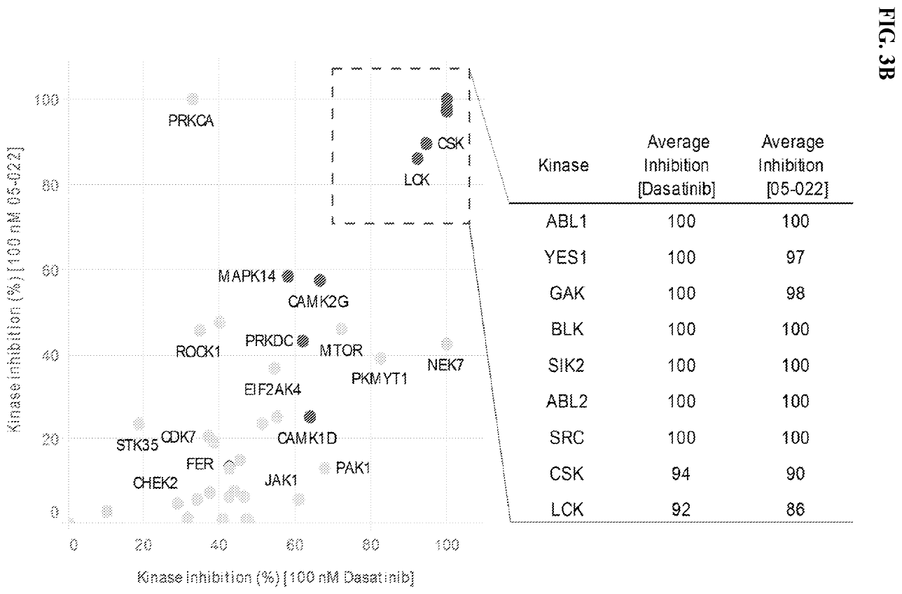

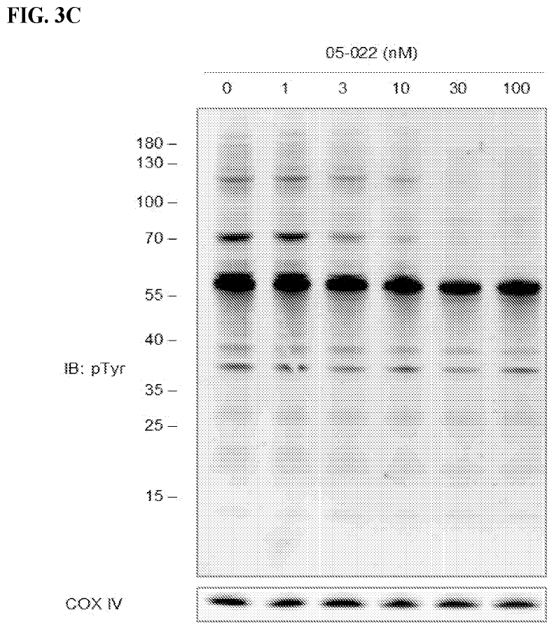

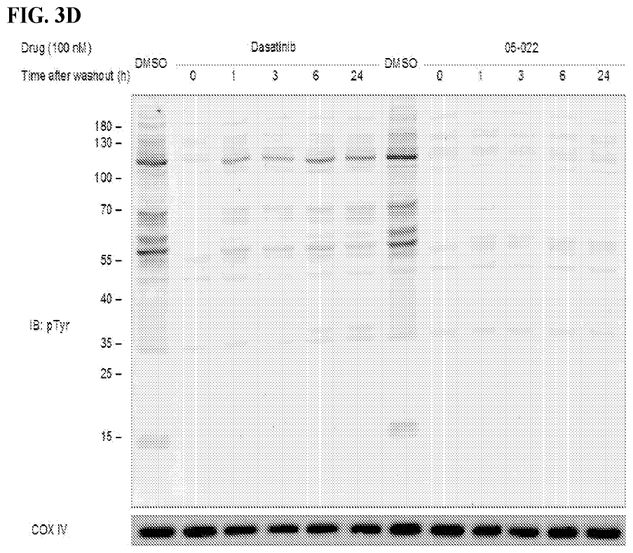

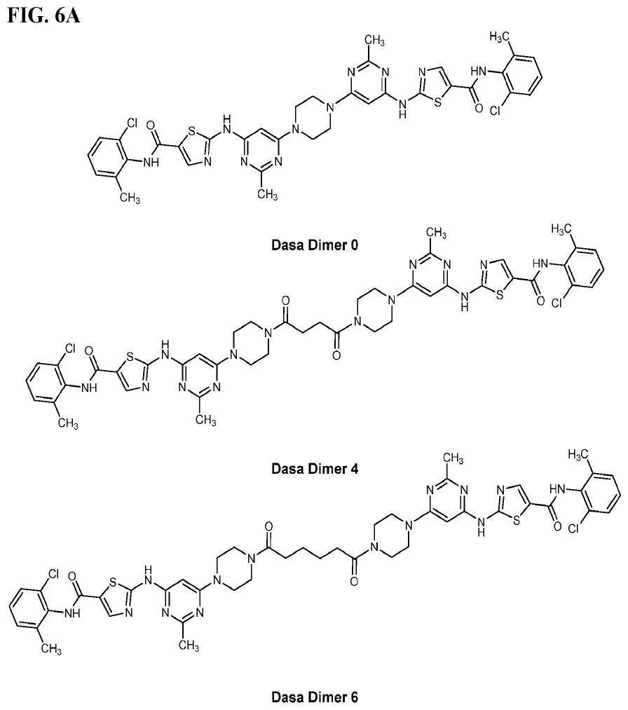

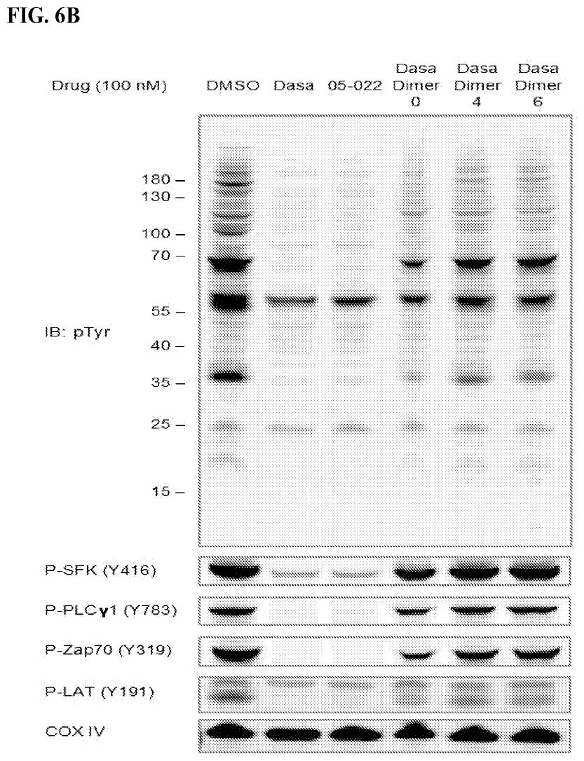

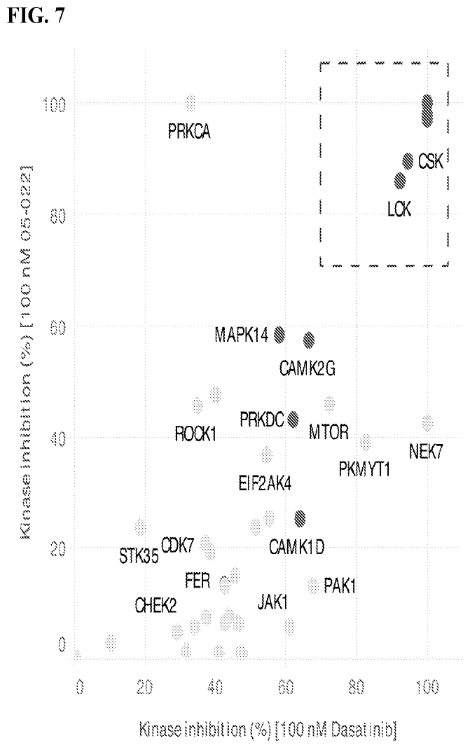

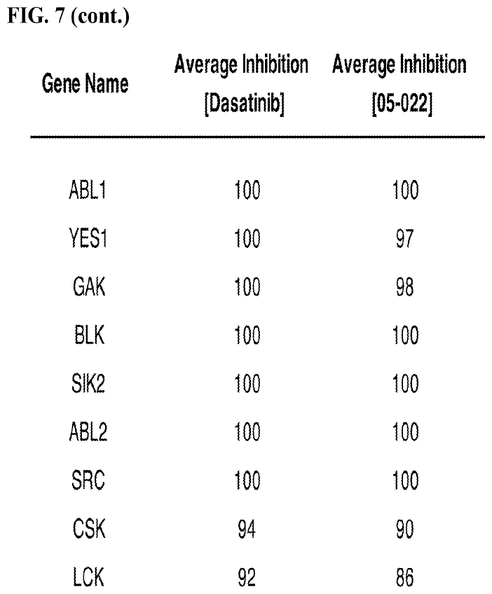

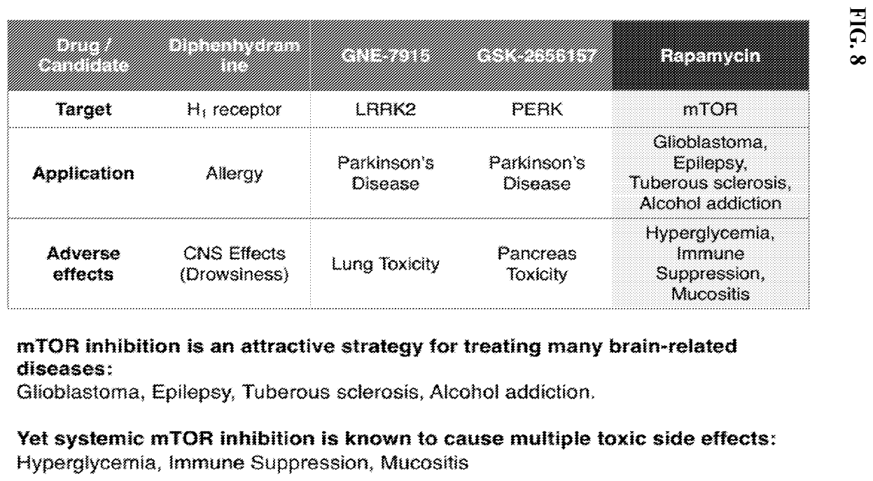

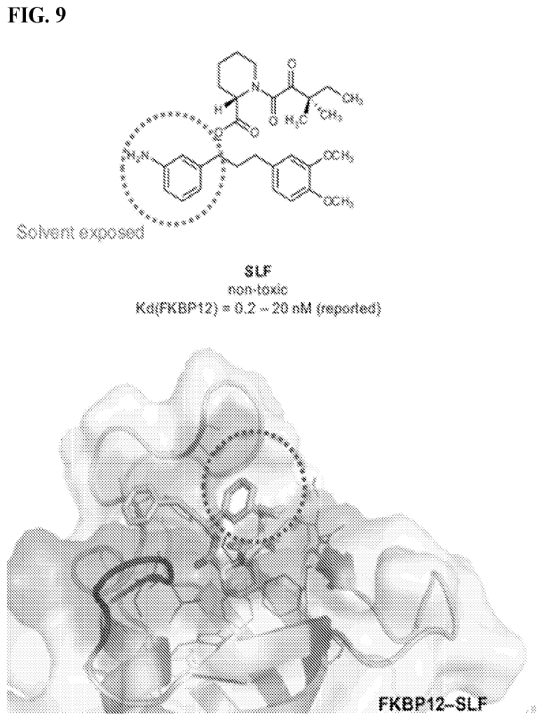

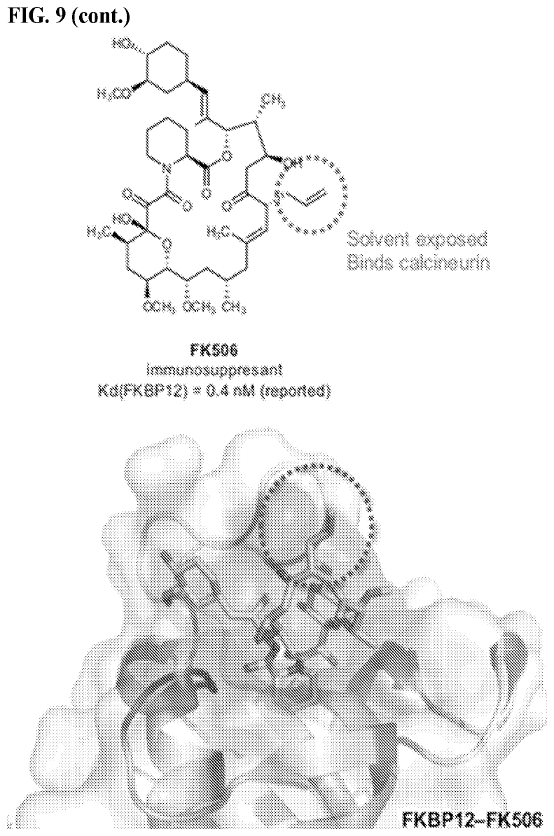

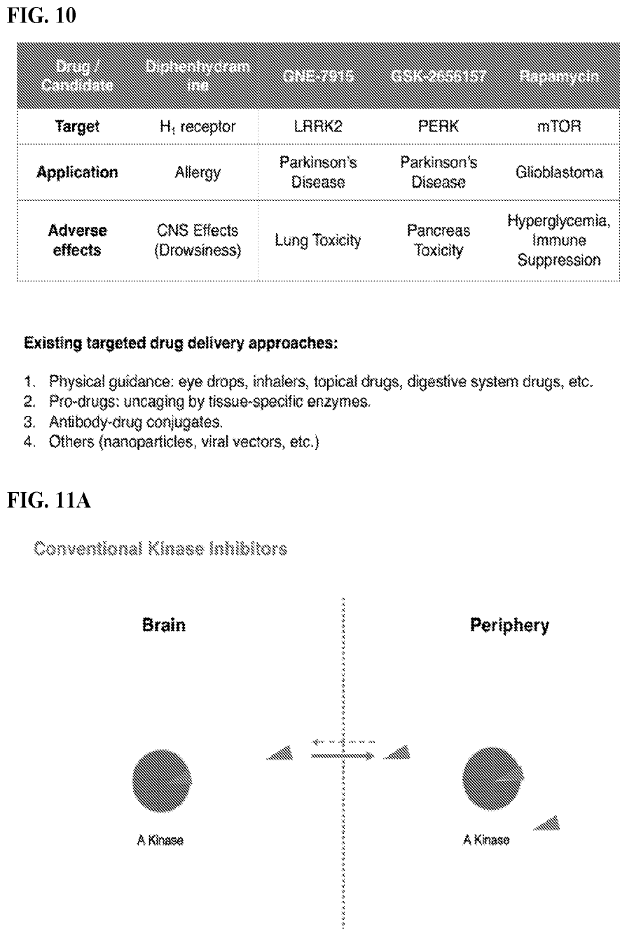

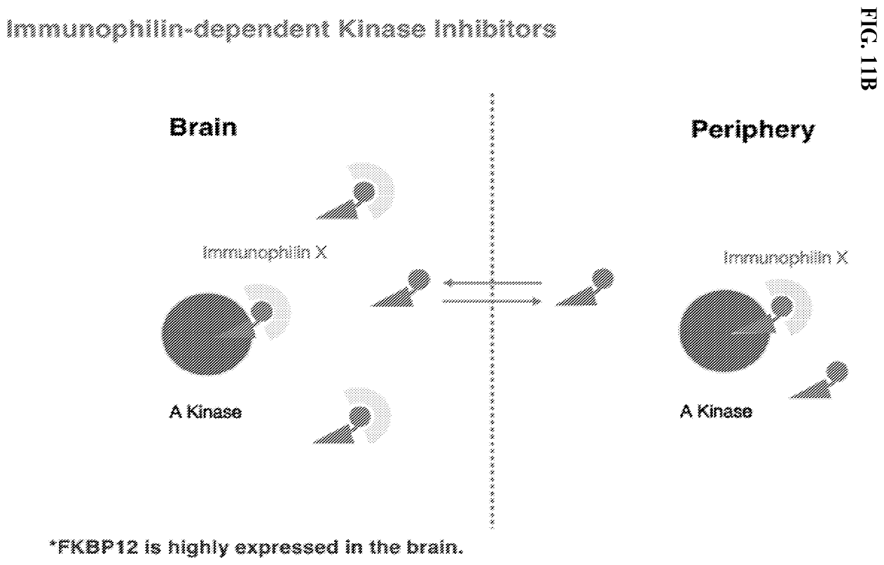

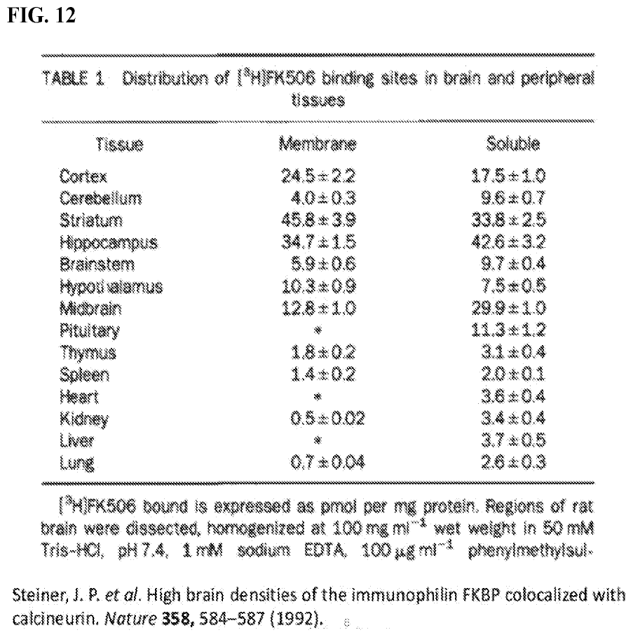

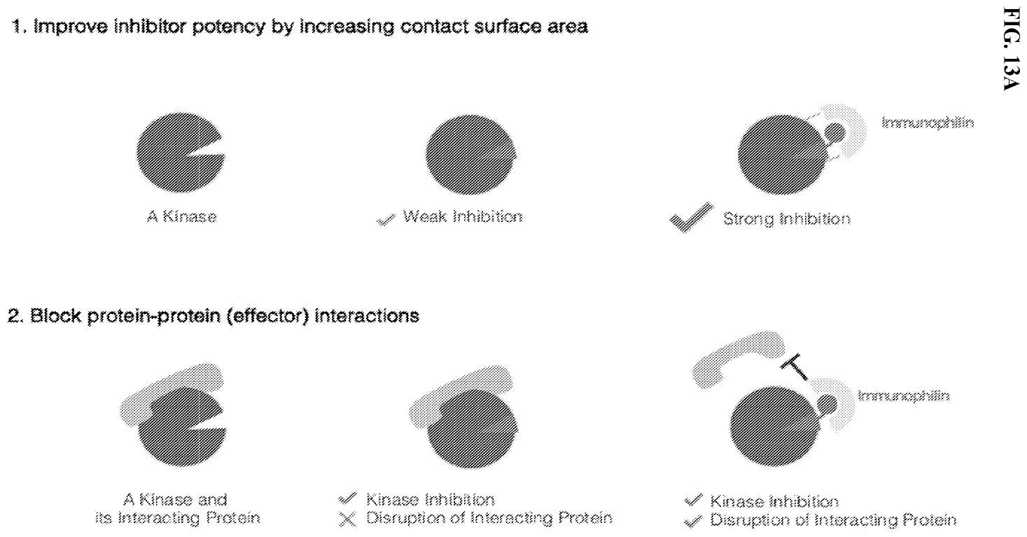

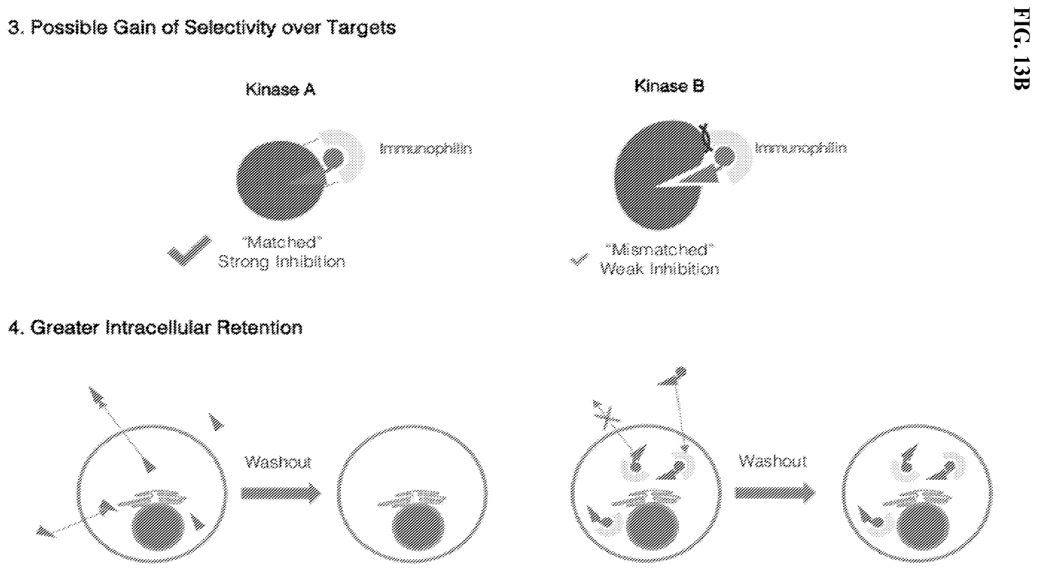

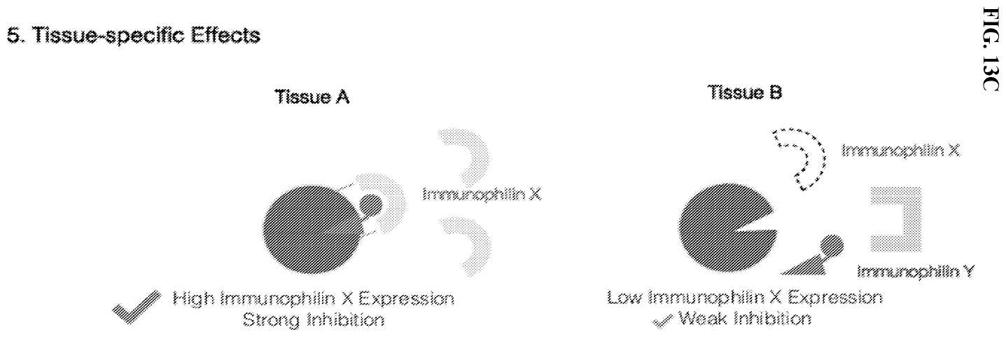

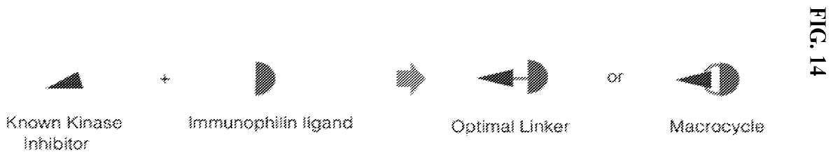

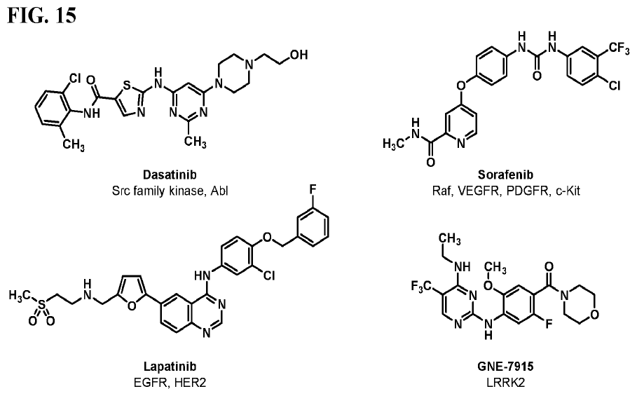

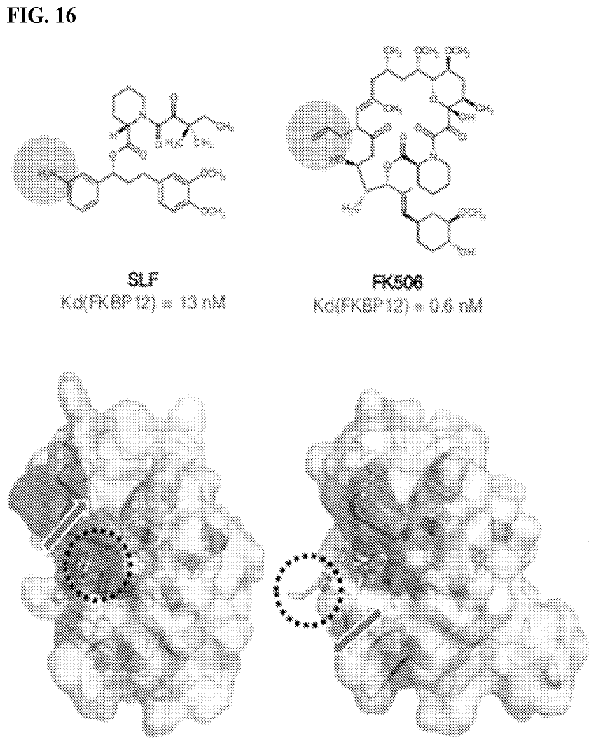

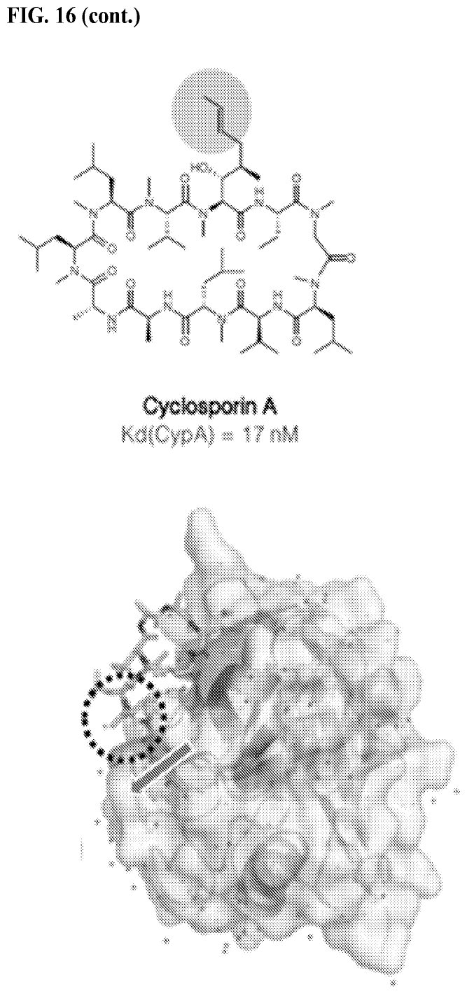

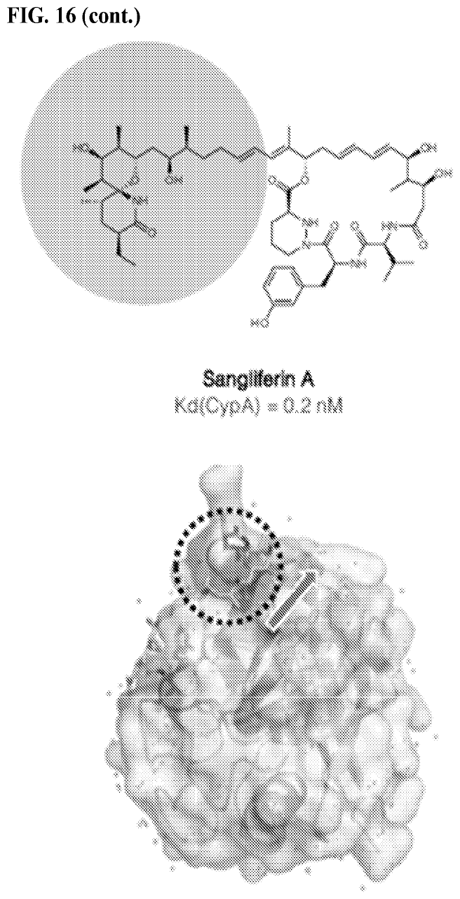

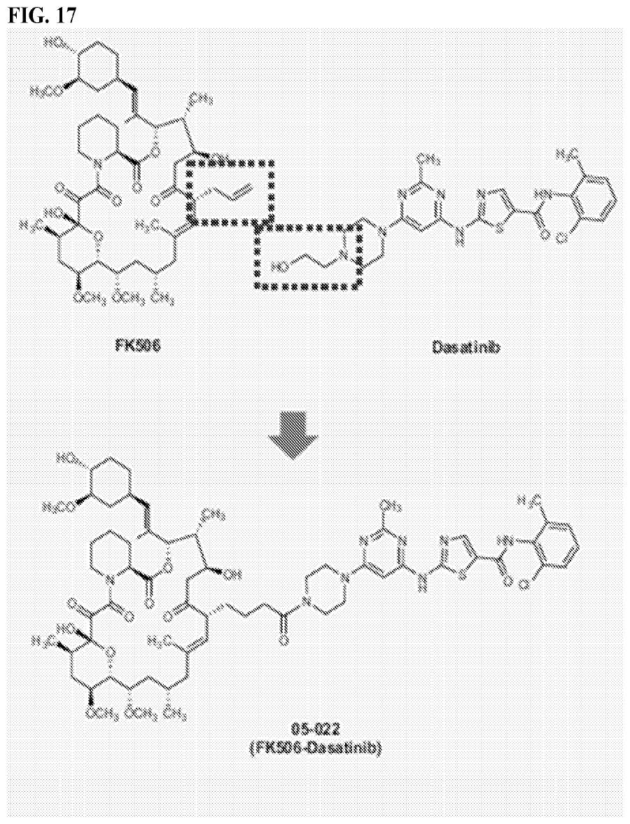

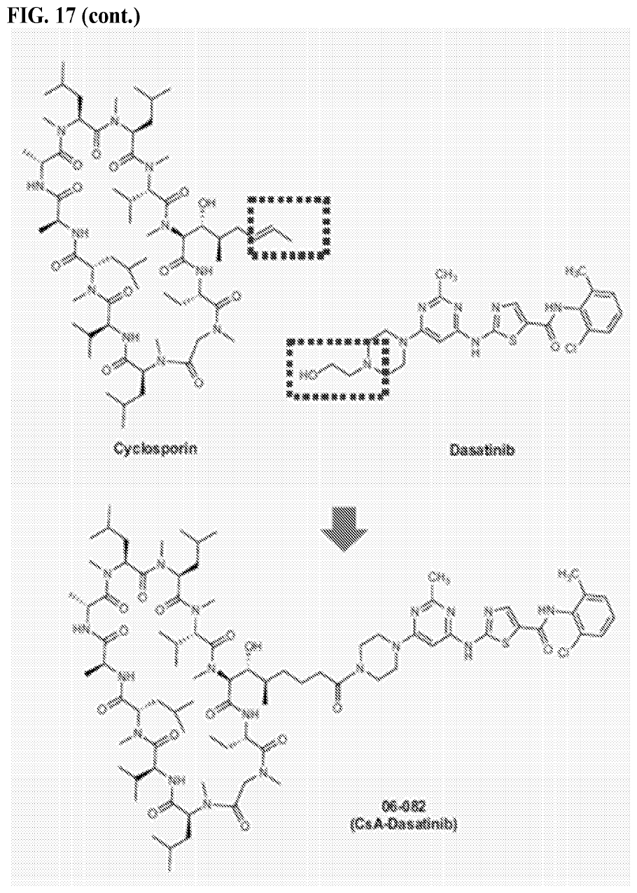

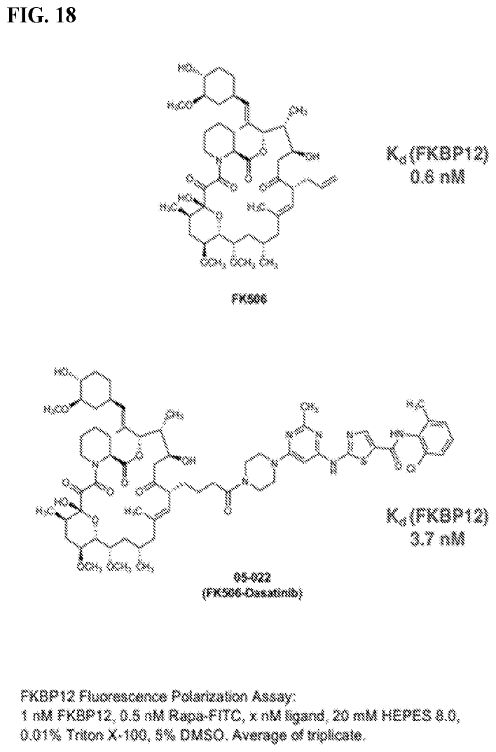

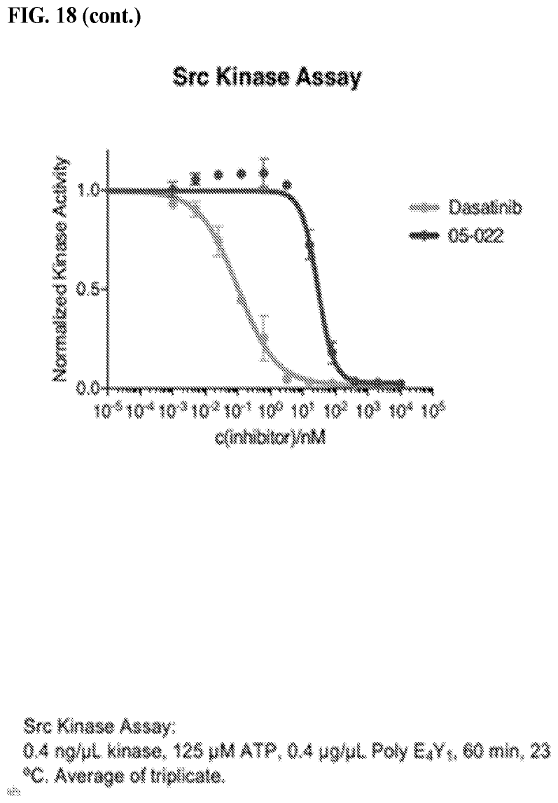

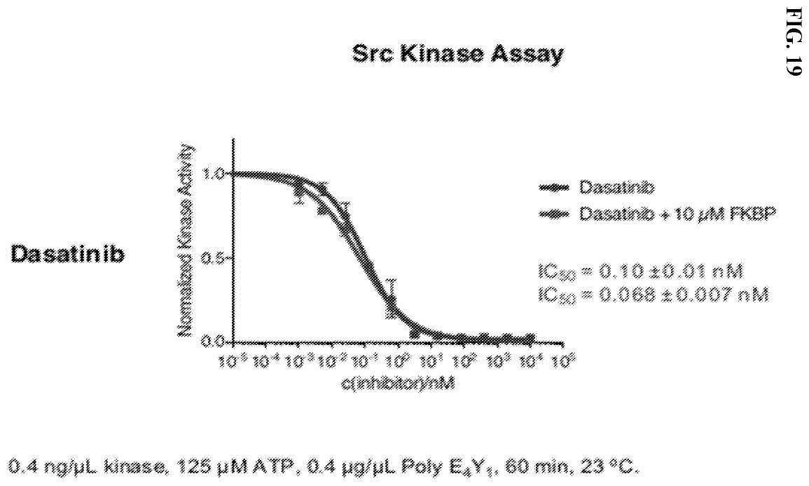

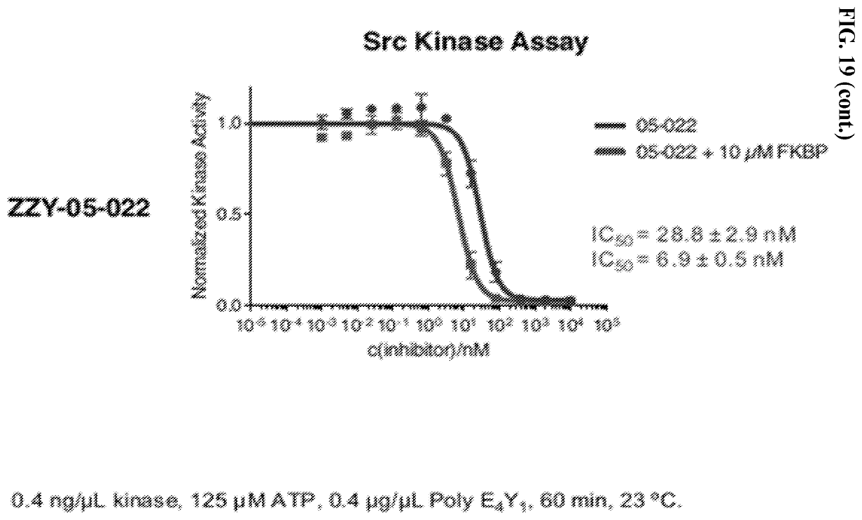

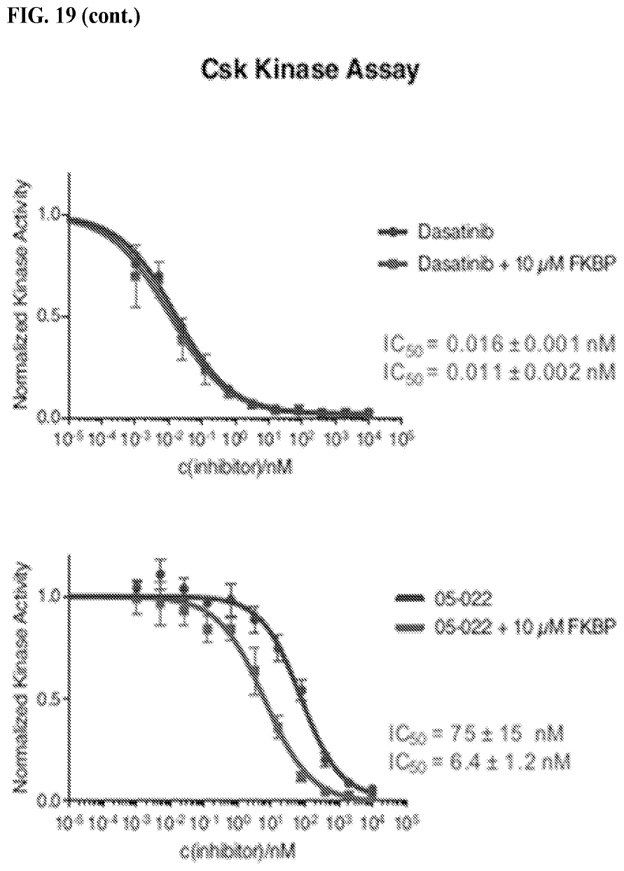

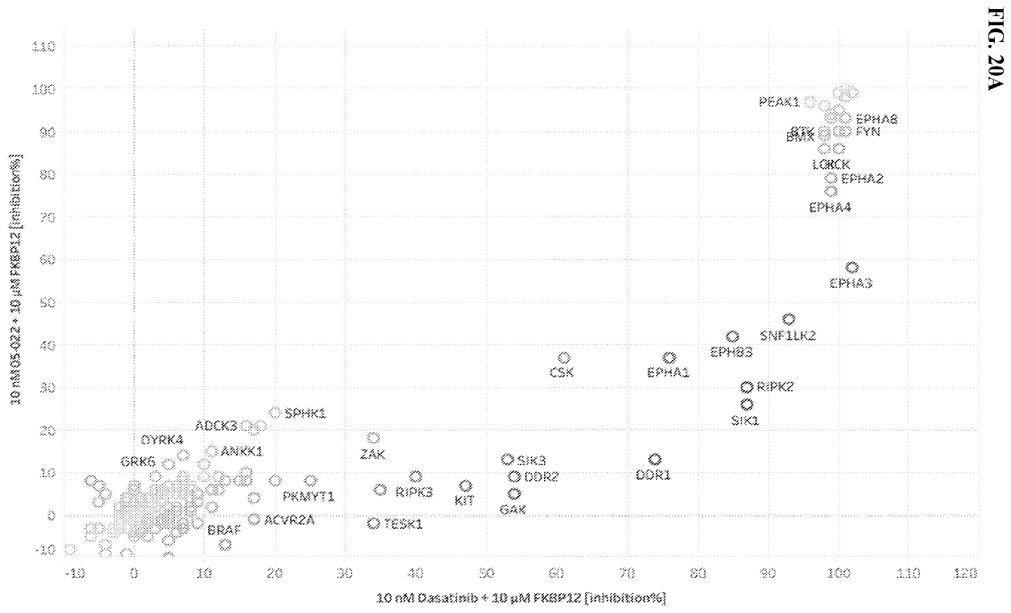

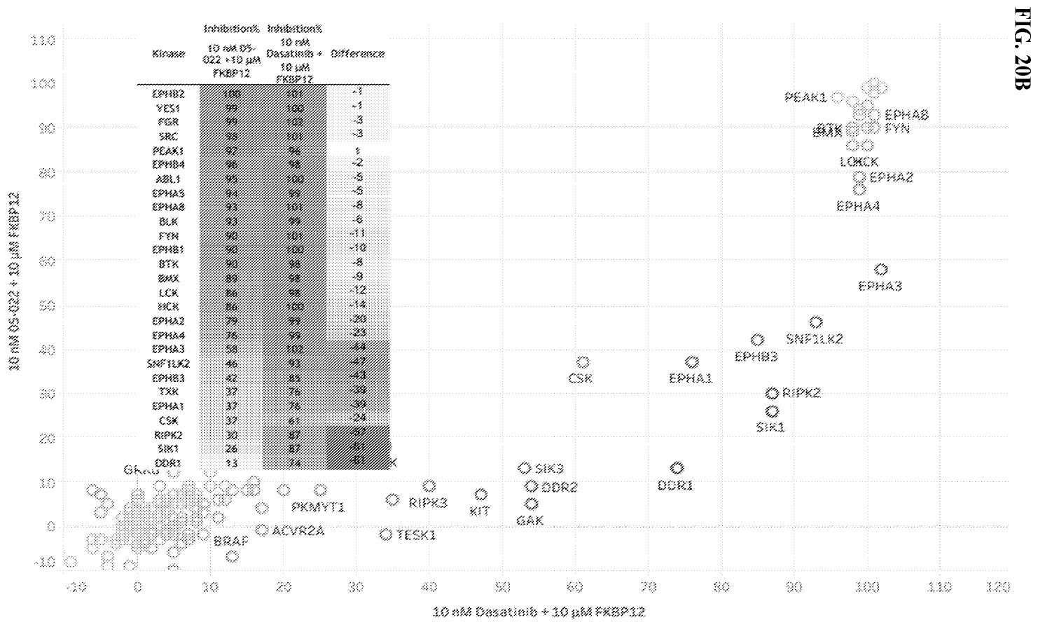

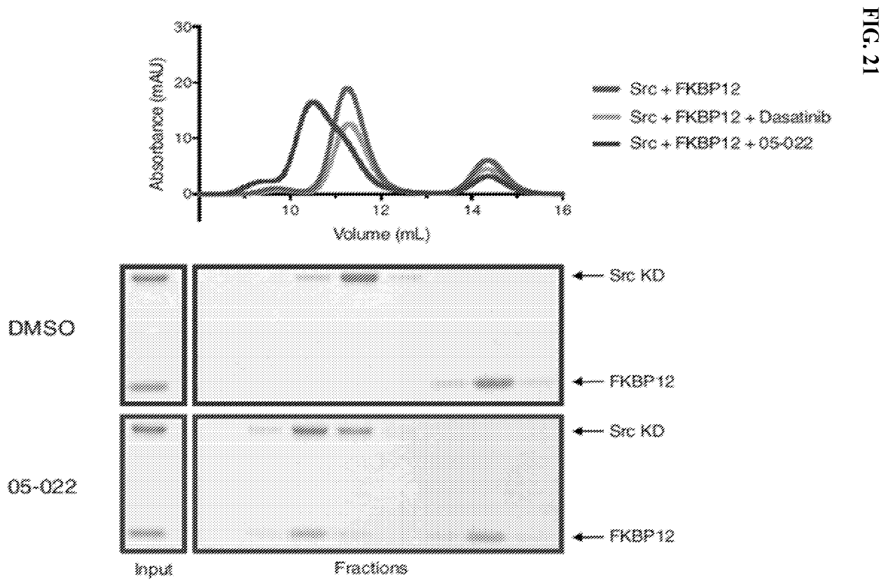

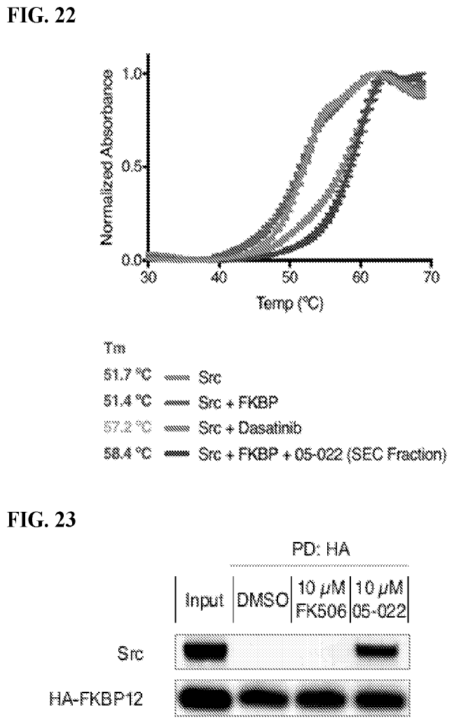

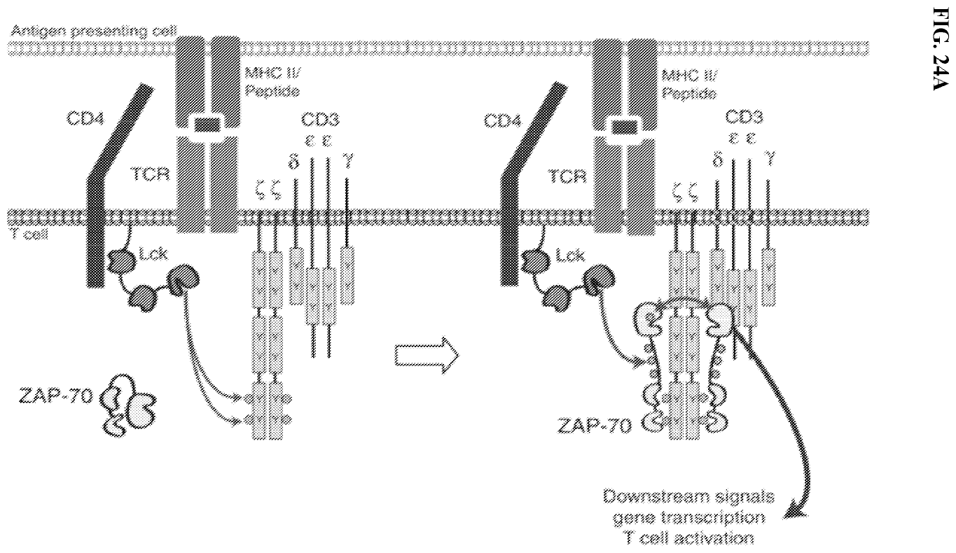

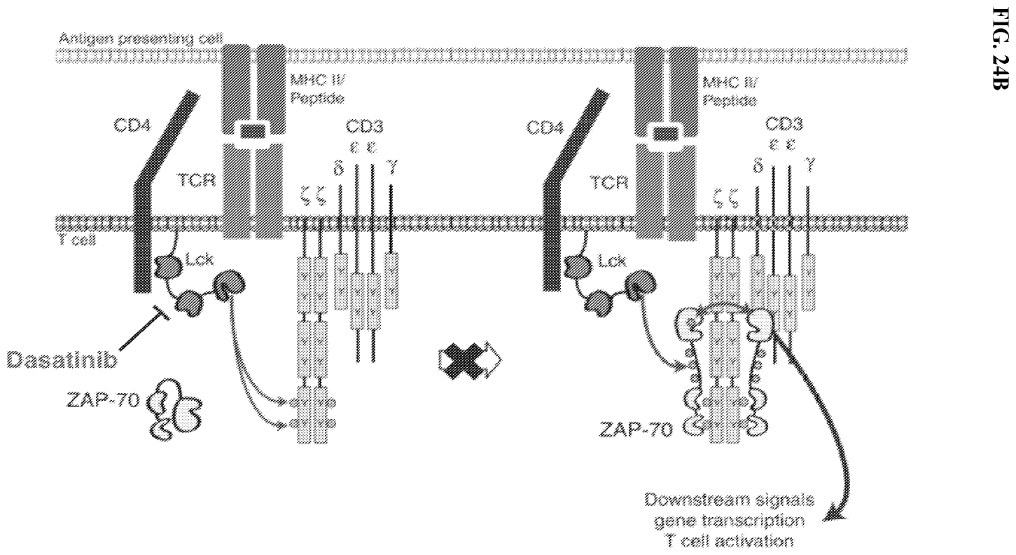

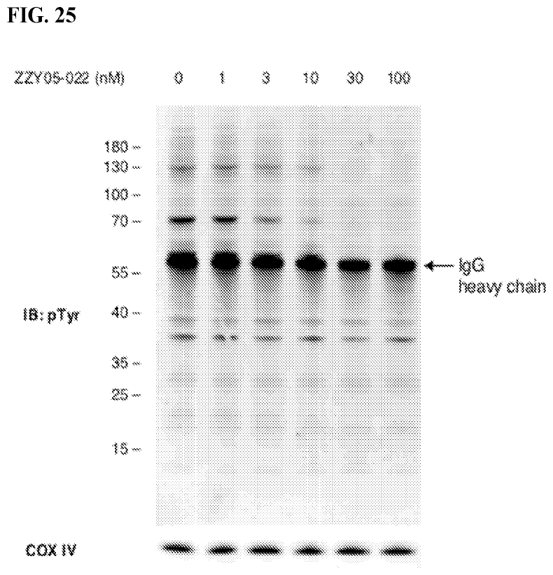

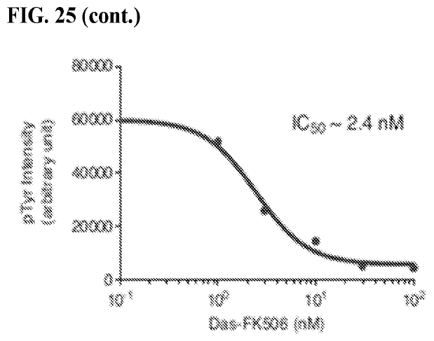

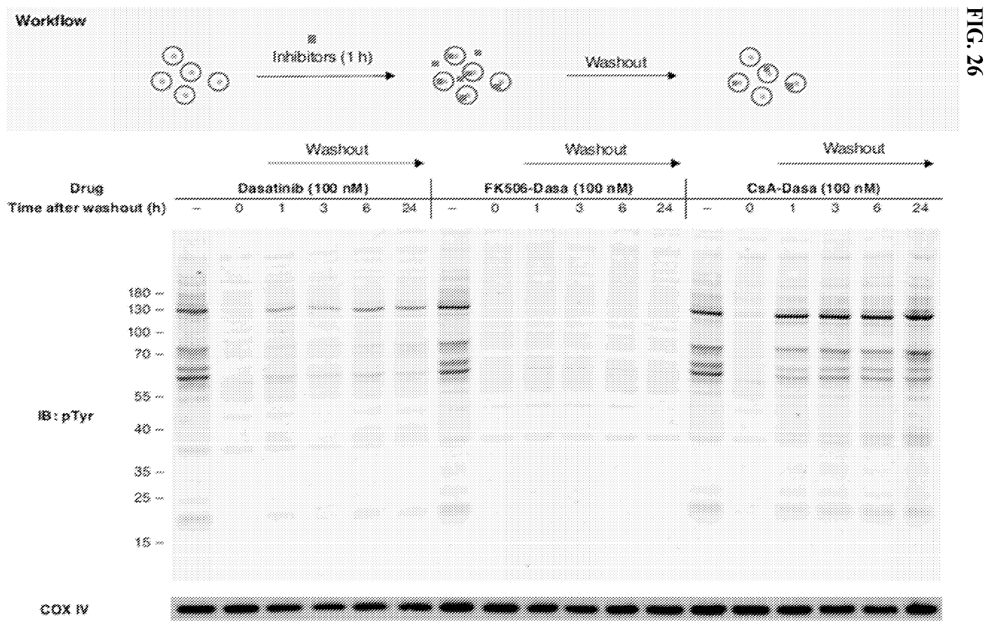

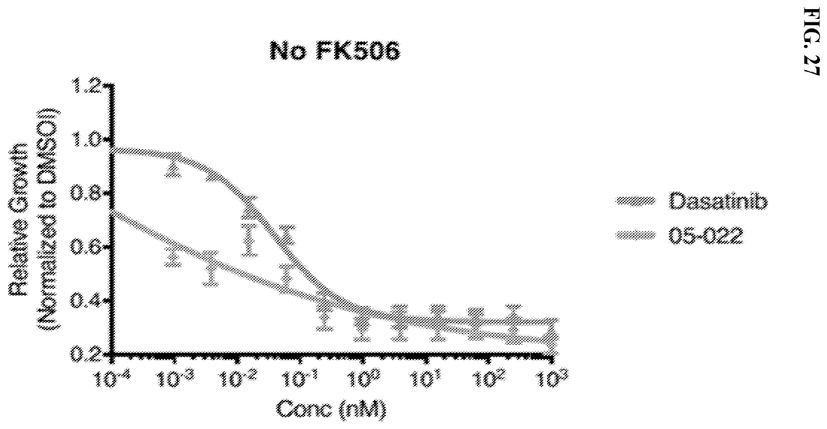

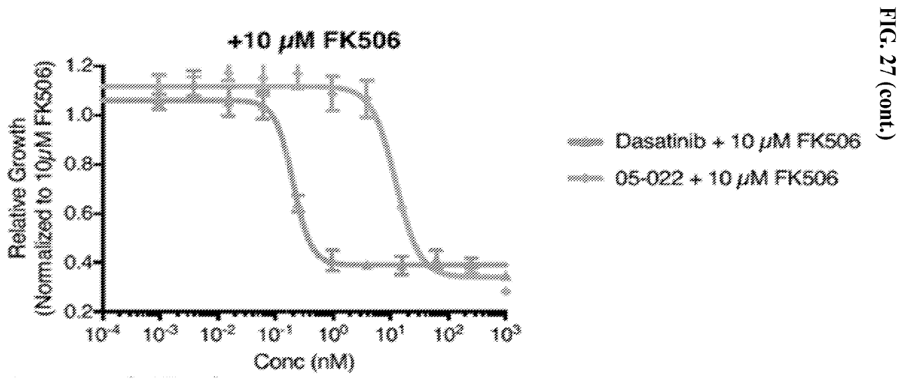

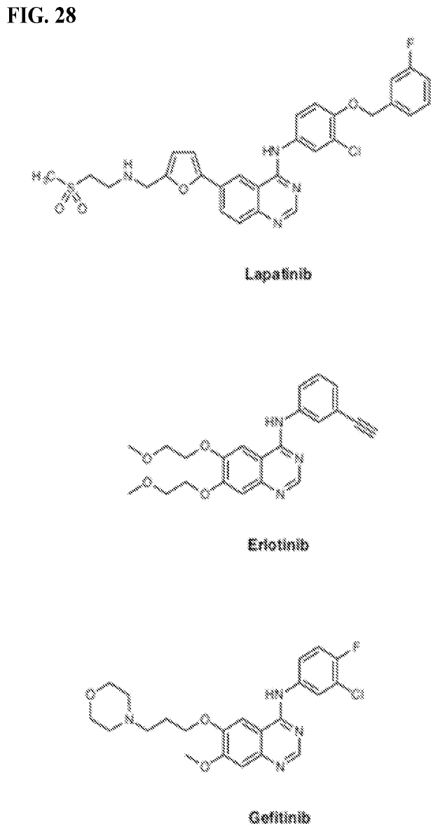

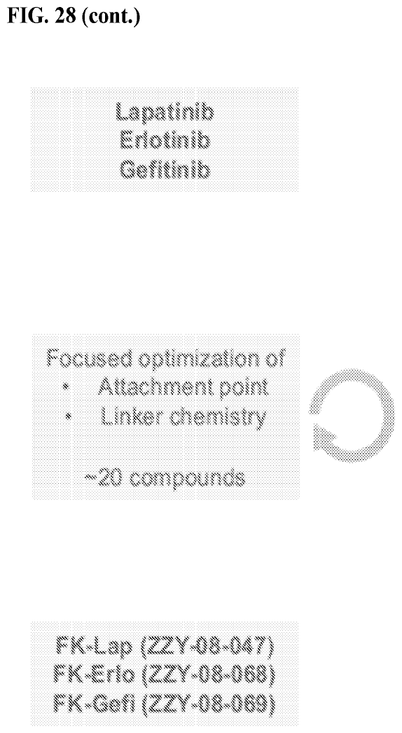

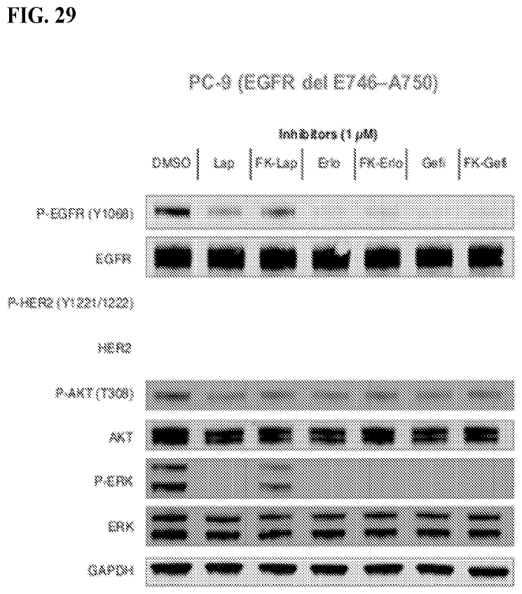

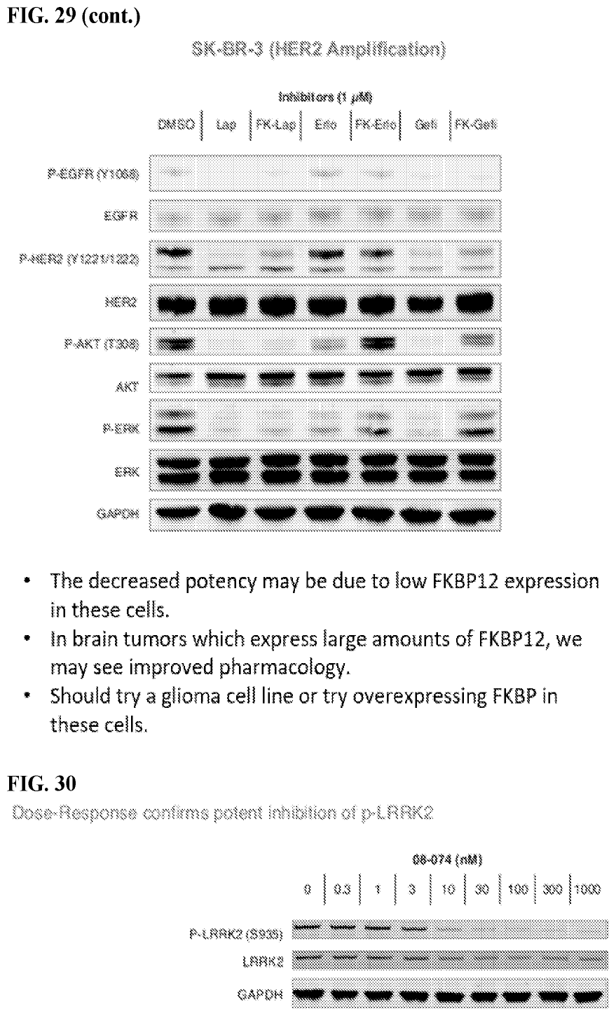

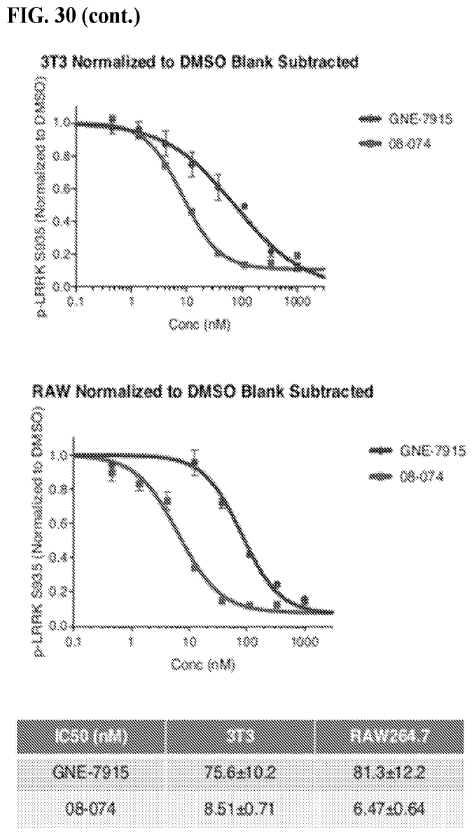

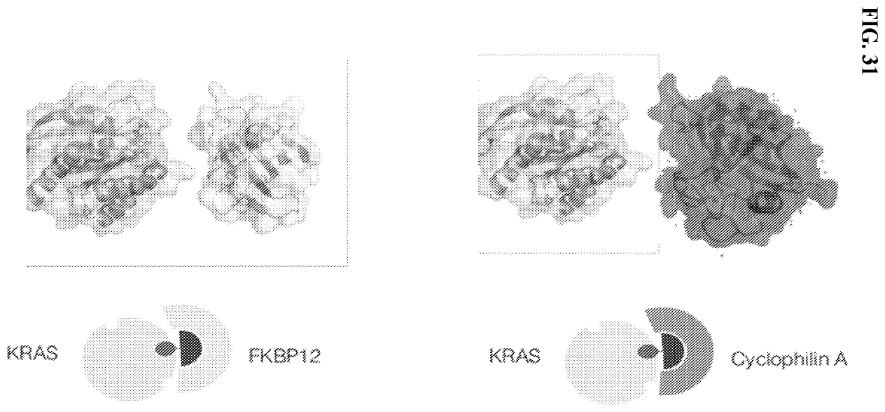

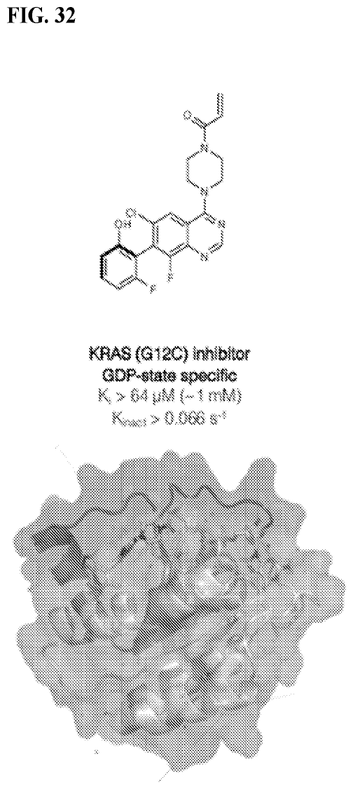

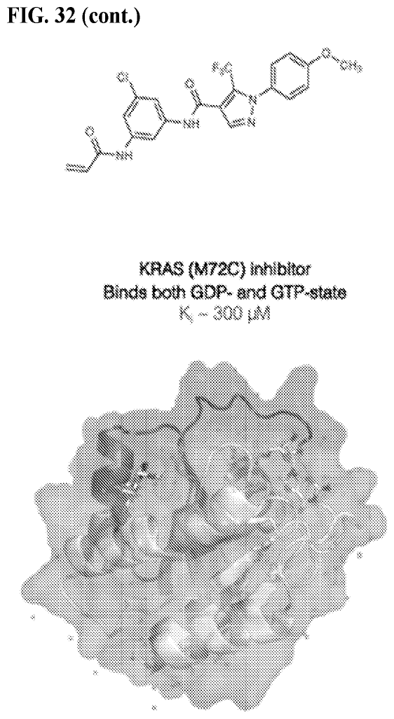

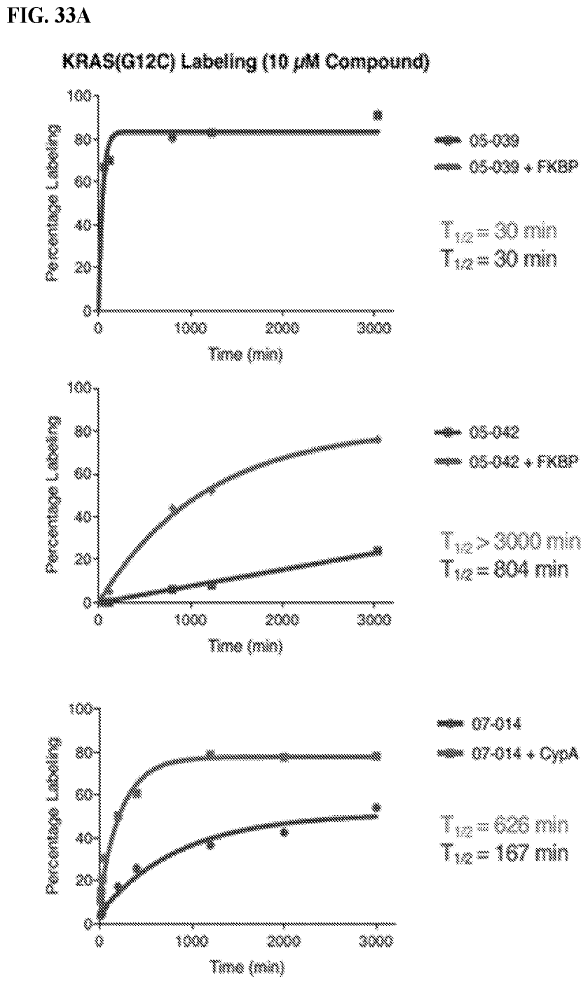

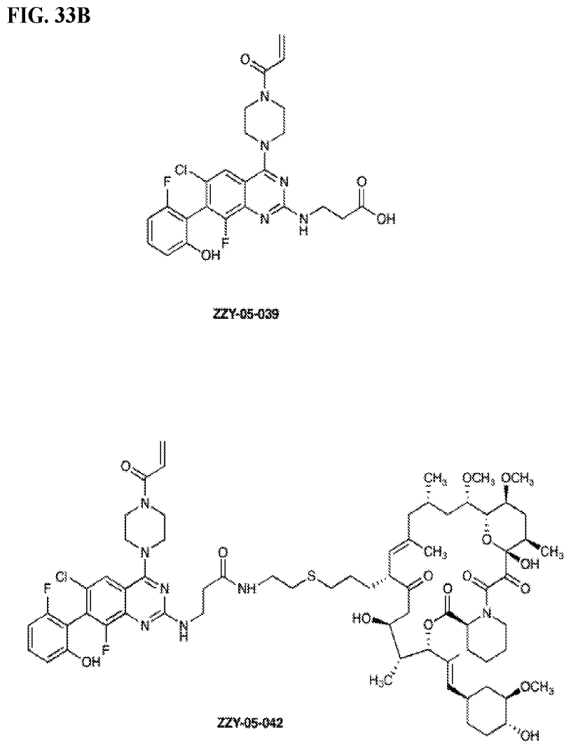

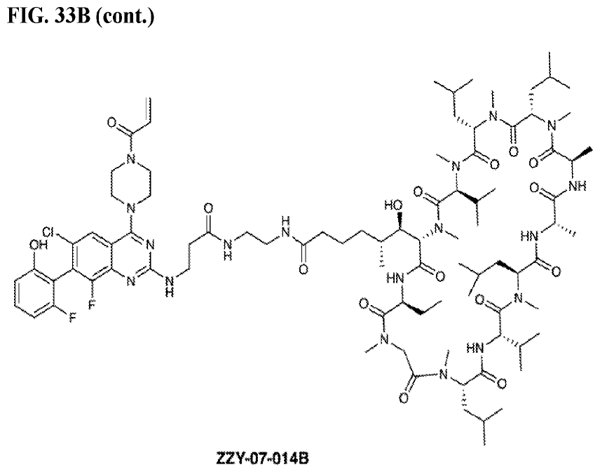

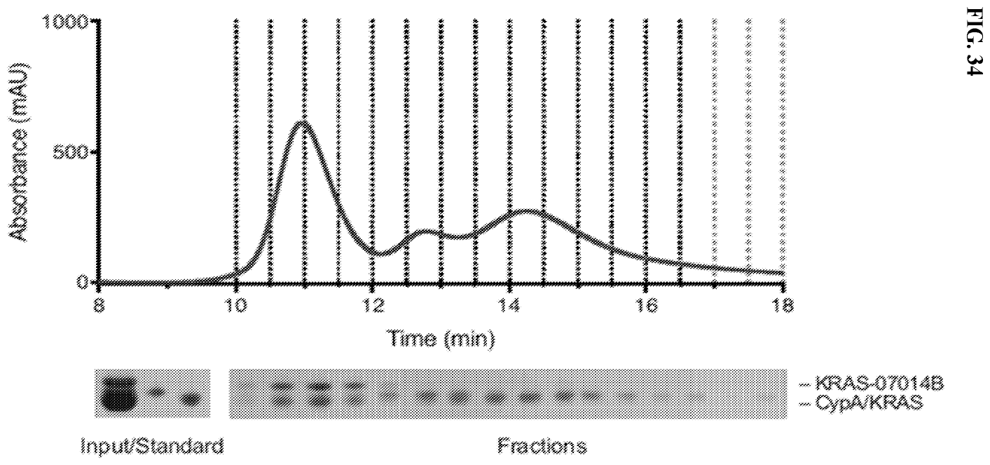

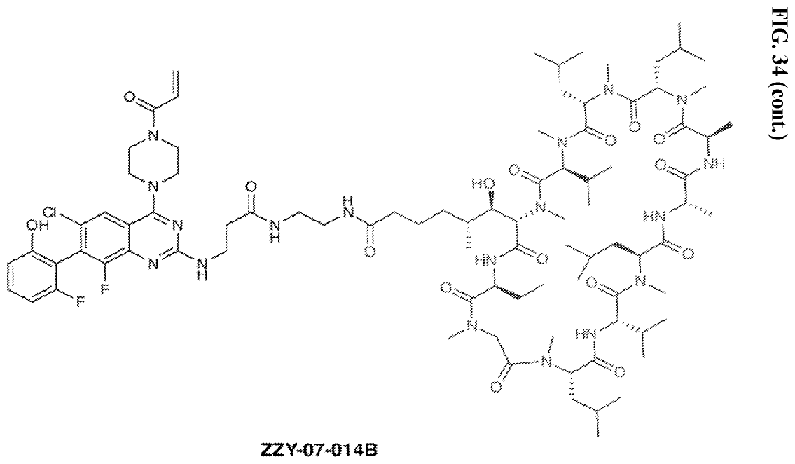

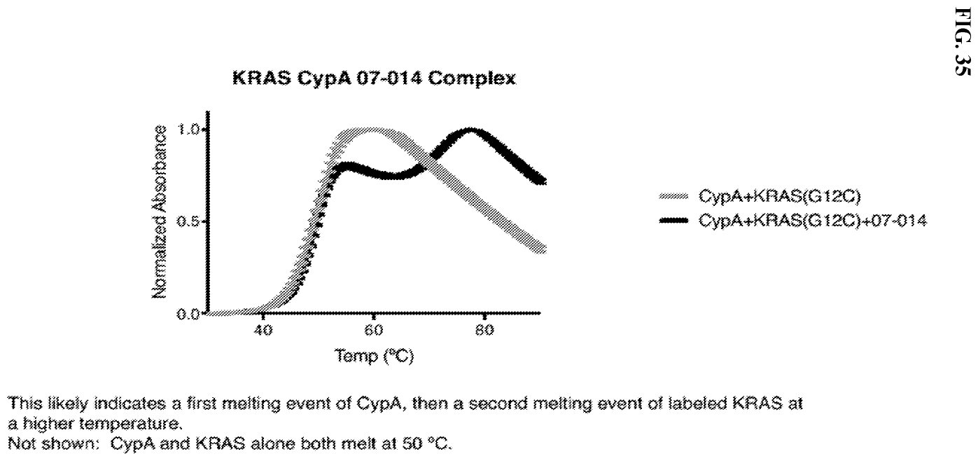

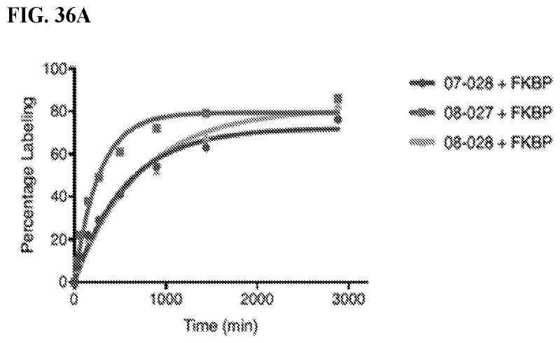

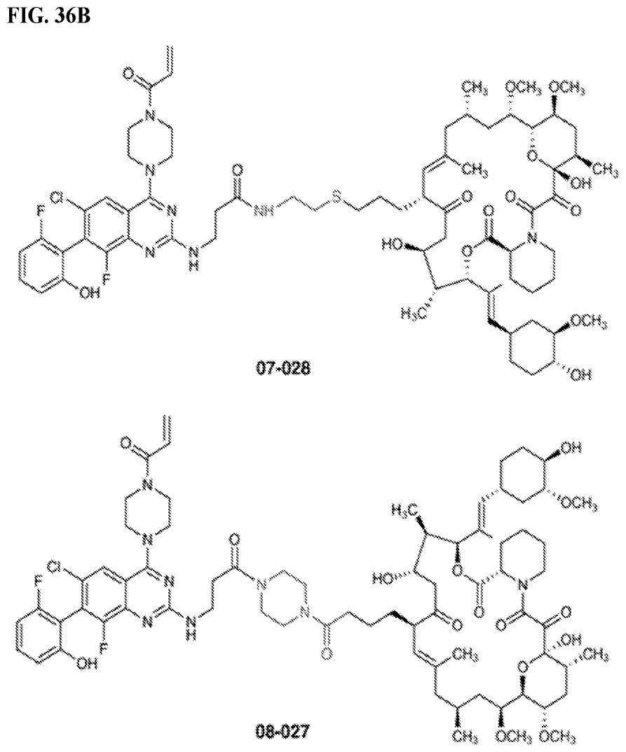

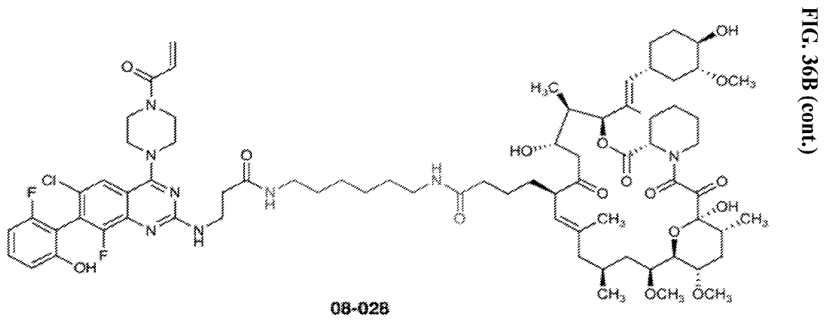

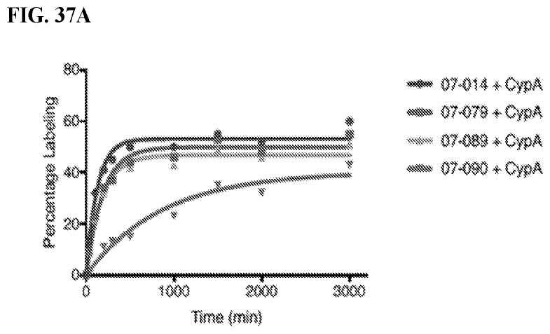

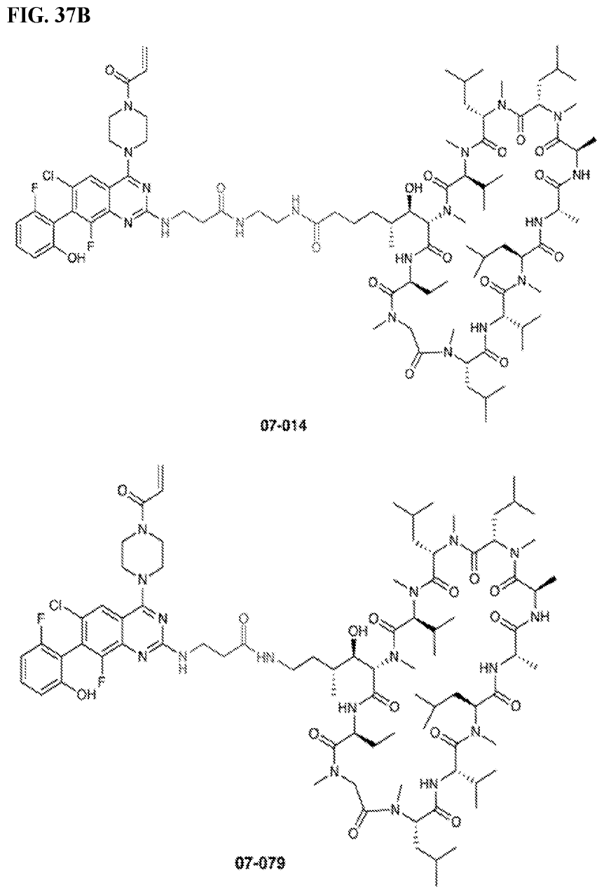

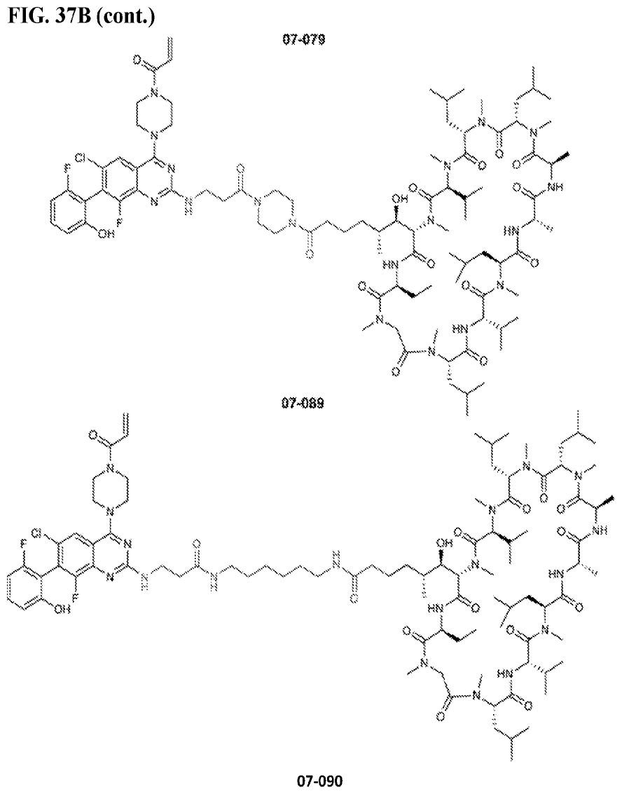

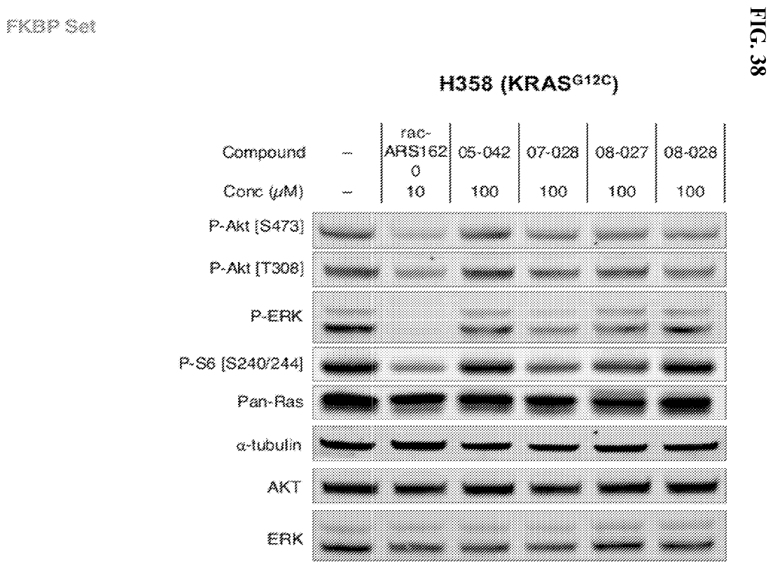

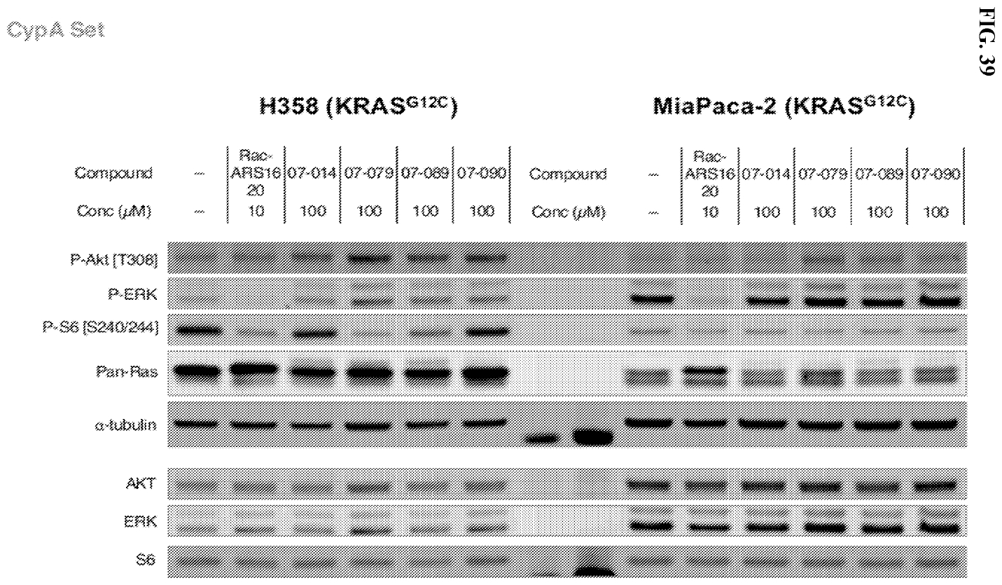

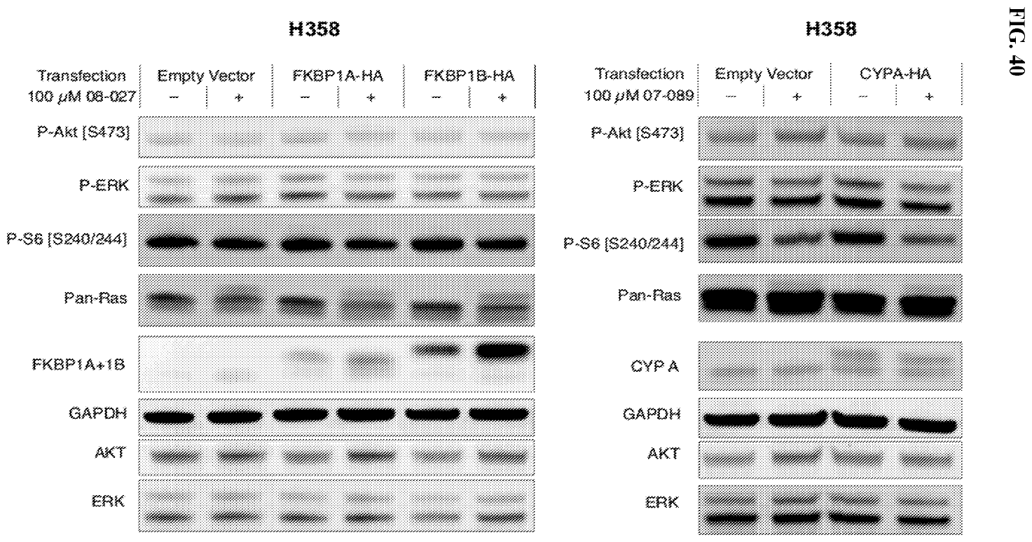

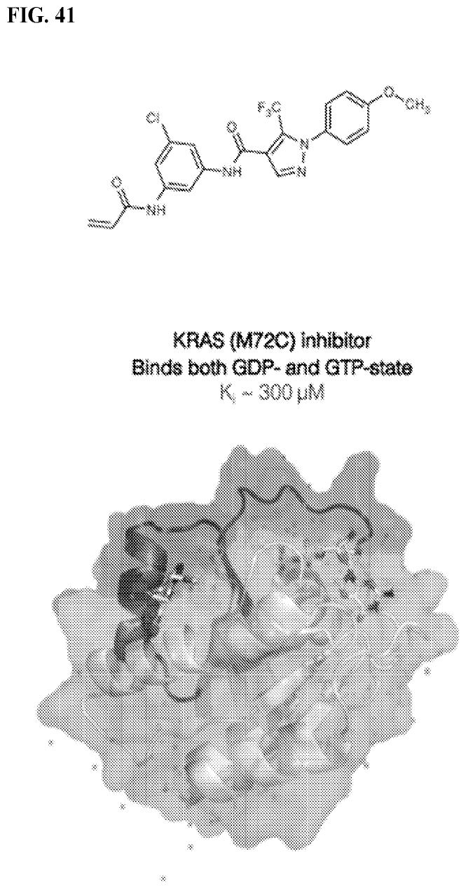

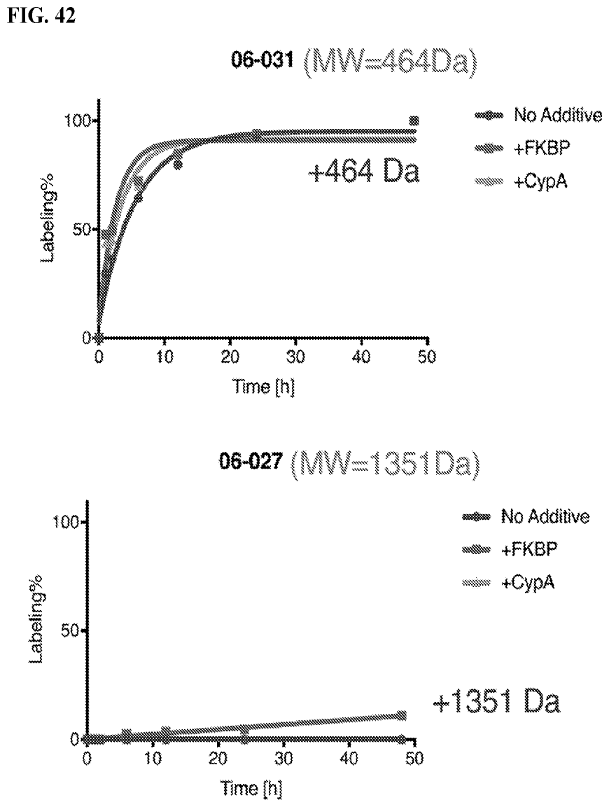

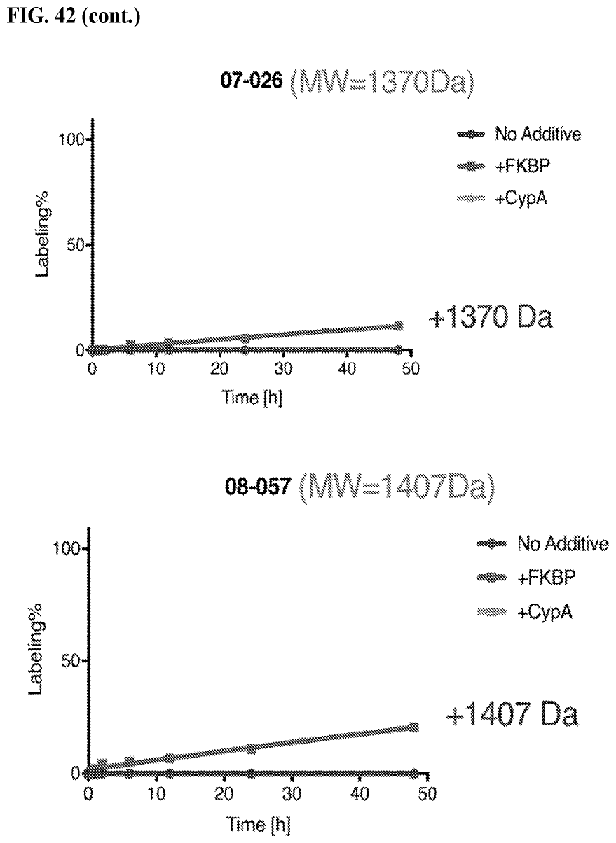

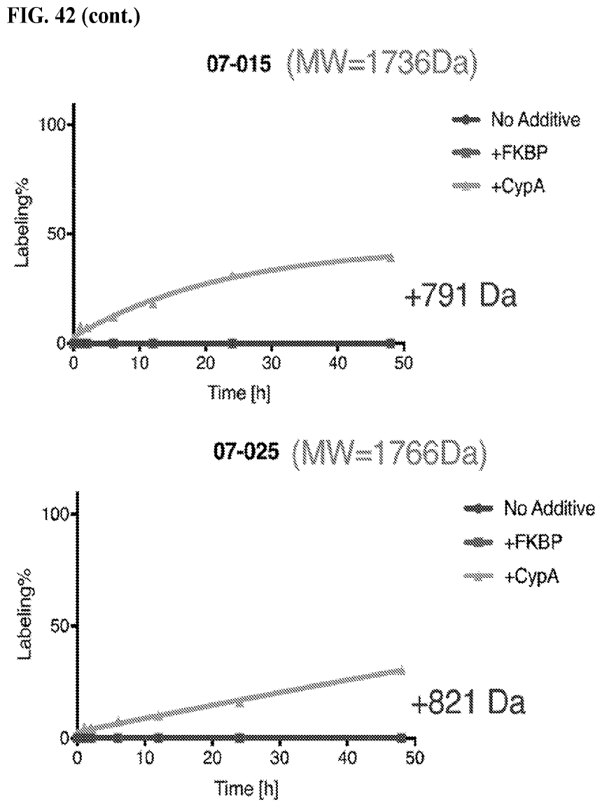

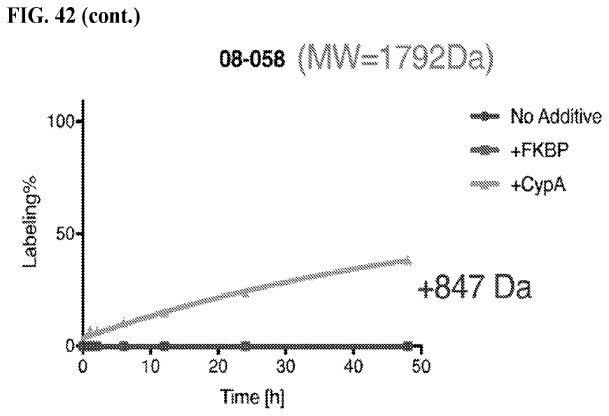

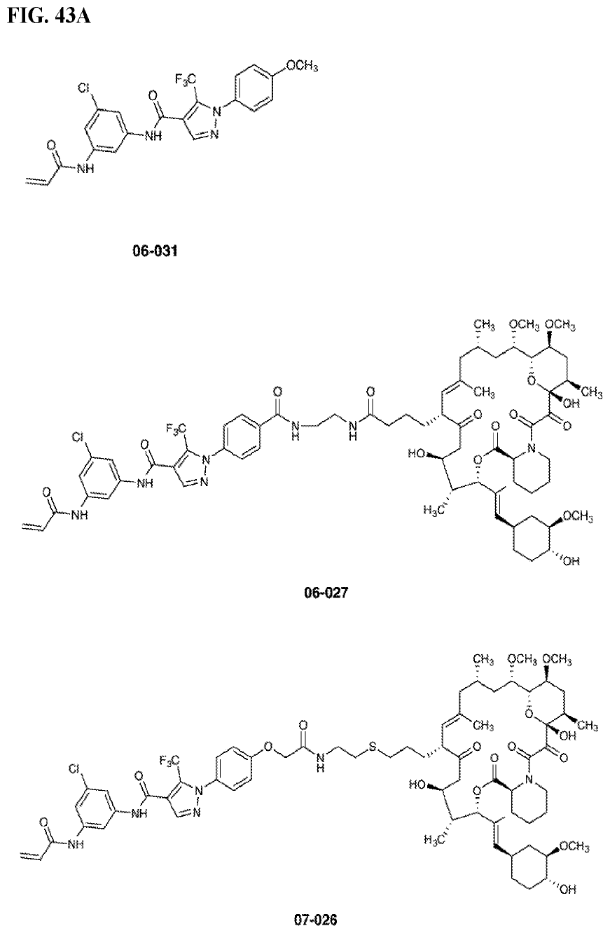

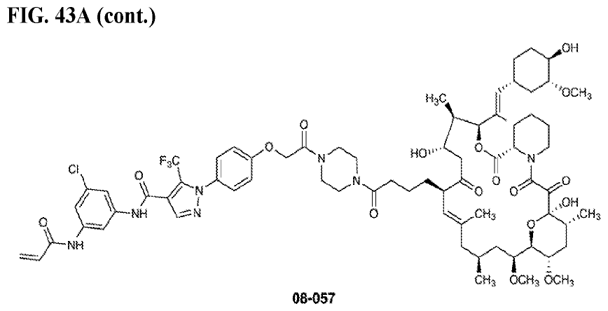

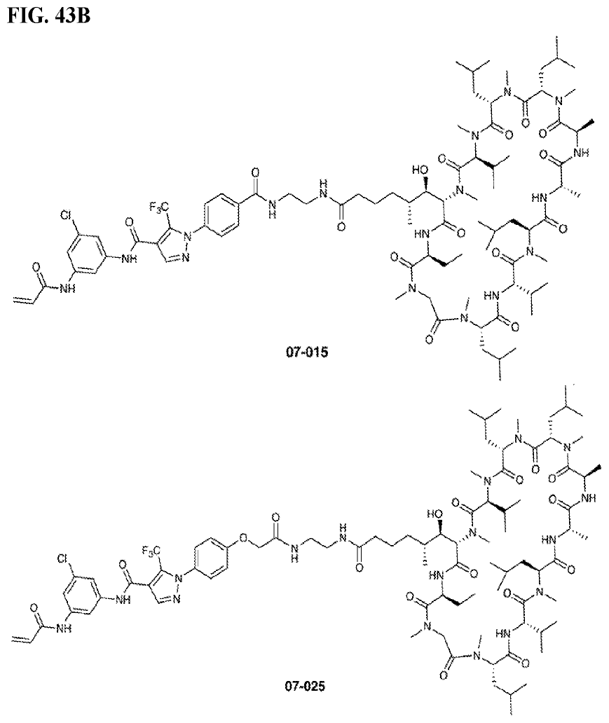

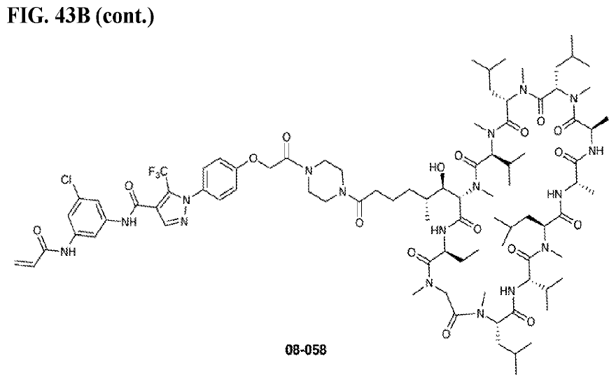

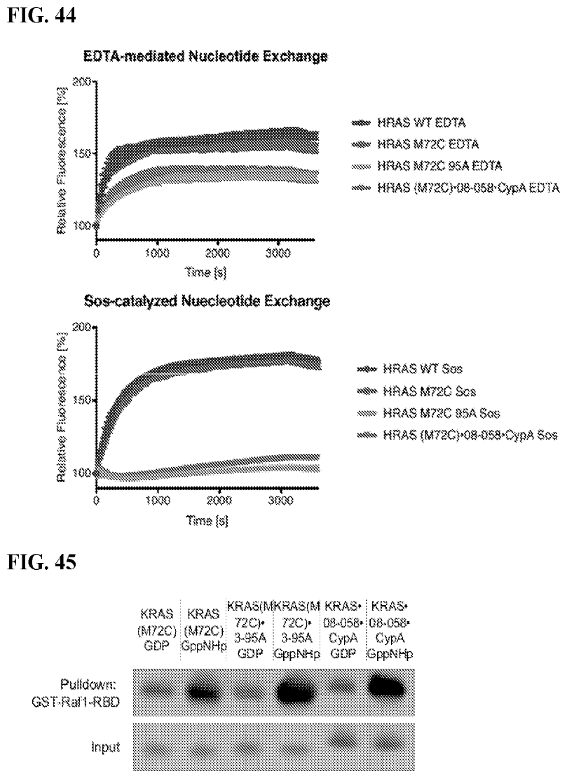

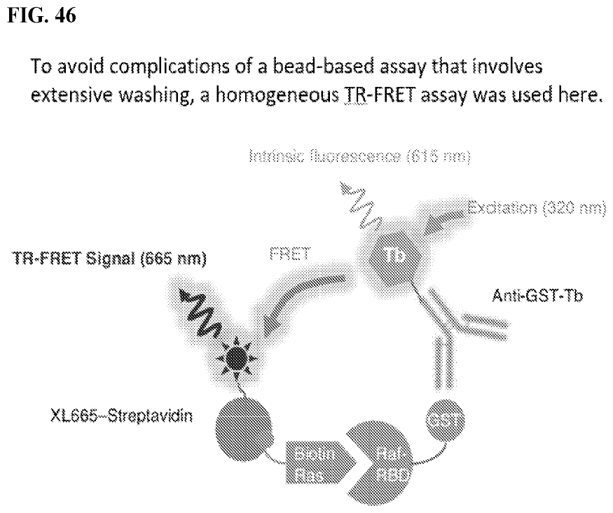

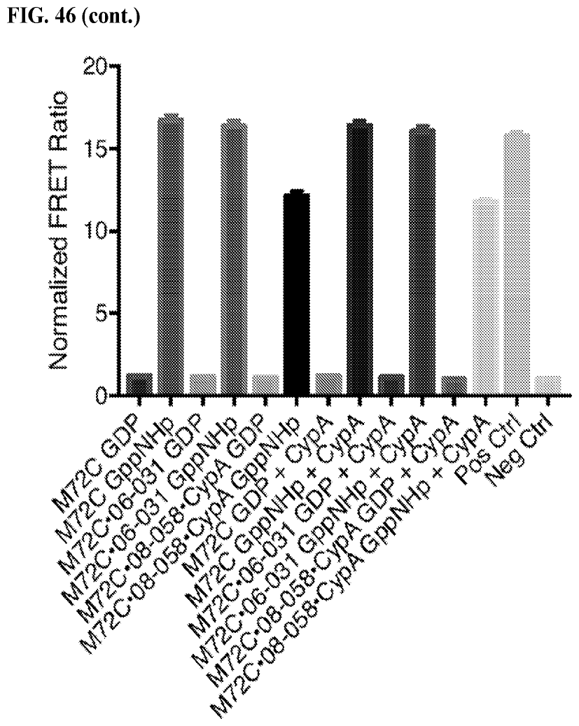

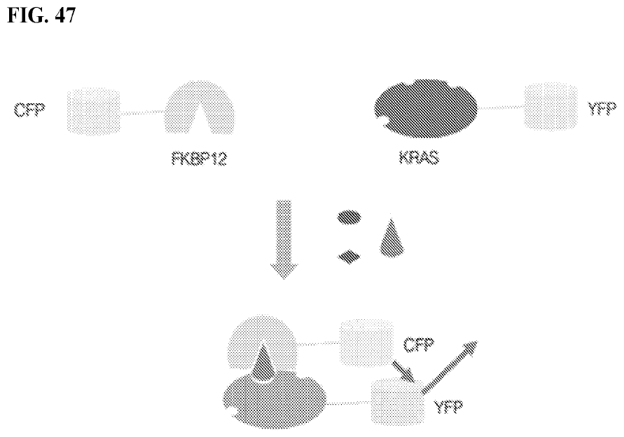

To evaluate the efficacy of FK-Dasatinib in cells, we studied its effect on CD3 crosslinking-triggered T cell activation. Src family kinases, notably Lck and Fyn, are key regulators of T cell receptor (TCR) signal transduction, and dasatinib is known to block T cell activation by inhibiting these kinases. We stimulated Jurkat cells with an anti-CD3 monoclonal antibody (OKT3) in the presence of dasatinib or FK-dasatinib and monitored their activation by Western blot. At 100 nM, both dasatinib and FK-dasatinib dampened the of total phospho-tyrosine level and suppressed the phosphorylation of several proteins involved in TCR signaling including Src-family kinases, PLCgamma1, ZAP70 and LAT (FIG. 3A). Interestingly, three homodimers of dasatinib containing linkers of various length (FIG. 6A) had no measurable inhibition of phosphotyrosine signal. FK-dasatinib was effective at concentrations as low as 10 nM (EC50=3.4 nM, FIG. 3C). To profile the target scope of FK-dasatinib in live cells, we employed a lysine-targeted chemoproteomic probe XO44, which irreversibly reacts with a conserved lysine in the ATP pocket of kinases and allows the quantification of the occupancy of the intracellular kinome by inhibitors by label-free mass spectrometry. We found that with both dasatinib and FK-dasatinib at 100 nM, an identical set of 9 kinases were inhibited >70% (FIG. 3B and FIG. 7) among the 139 kinases captured by the probe. This is consistent with previous knowledge of dasatinib as well as our findings in the biochemical profiling with purified kinases. That the selectivity of FK-dasatinib was indistinguishable from dasatinib was not surprising—none of kinases displaying differential response to the two inhibitors in the biochemical assay (FIG. 1C) were highly expressed in Jurkat cells or detected by the XO44 probe. Notwithstanding, one remarkable distinction of FK-dasatinib from dasatinib we observed was its prolonged residence time in cells. We measured the change of phosphotyrosine levels at various timepoints after treating Jurkat cells with 100 nM dasatinib or FK-dasatinib for 1 h and removing the drug (FIG. 3D). Restored phosphotyrosine bands were seen at as early as 1 h in dasatinib-treated cells. By contrast, no increase in phosphotyrosine signals could be detected even at 24 h after the drug washout in FK-dasatinib-treated cells, suggesting a mechanism that supports durable cellular retention of the drug. Unusually long cellular retention times have also been previously observed with other drugs that engage FKBP proteins. We believe that in these cases, the abundant intracellular FKBP proteins serve as a sink for these drugs, capturing them as in a FKBP-drug complex that cannot cross the plasma membrane to exit the cell and hence significantly lengthening their residence inside the cell.

### Example 4: Design of Ligands Based on EGFR and LRRK2 Inhibitors

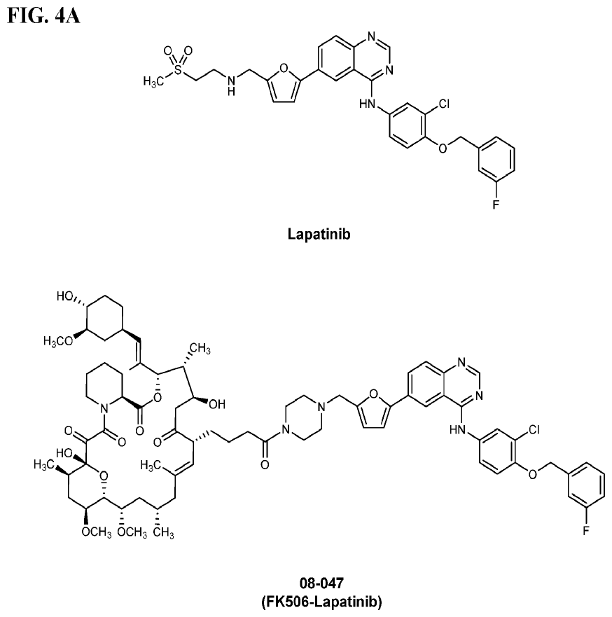

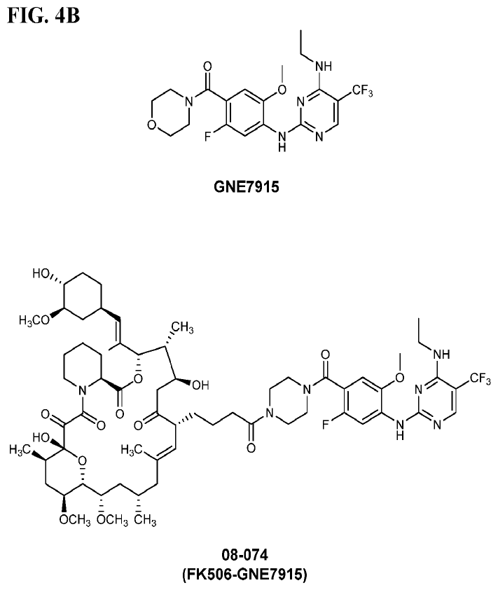

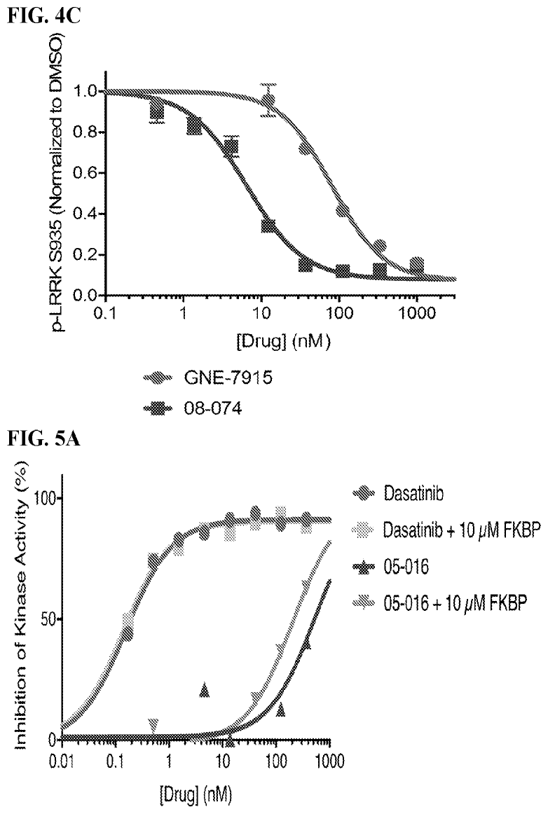

We extended our design strategy and prepared two other bispecific ligands based on the structures of an FDA-approved HER2/EGFR inhibitor (lapatinib) and an LRRK2 inhibitor in clinical development (GNE7915). FK-lapatinib suppressed HER2 signaling in SK-BR-3 cells (a cell line with HER2-amplification) at 1 μM and inhibited the growth of this cell line with an IC50 comparable to that of lapatinib (FIG. 4A). Interestingly, FK-GNE7915 appeared to be more efficacious than its parent molecule GNE7915 at inhibiting the autophosphorylation of LRRK2, a kinase currently pursued as a promising therapeutic target for Parkinson's disease. With the three case studies presented above, we cautiously anticipate that the same workflow could be applied to convert other kinase inhibitors into FKBP-dependent formats with similar if not better cellular activities.

By chemically linking FK506 and ATP-site kinase inhibitors at their respective solvent-exposed sites, we have developed a method to build a new class of kinase inhibitors whose activity depends on an endogenous protein, FKBP12. These inhibitors are characterized by their ability to mediate the formation of a ternary complex of the drug, the target kinase and FKBP12. While we have focused on protein kinases in this study, it seems reasonable to expect that the approach is also applicable to other classes of therapeutic targets, such as GTPases and histone modification enzymes.

### Example 5: Experimental Procedures

Note on rotamers in ¹H NMR data: All of the SLF analogs and FK506 analogs synthesized here exist as a mixture of two amide rotamers in CDCl₃ or CD₃OD. Due to extensive spectral overlap of the two, the coupling pattern of certain protons can be complicated even if they should display clear splitting patterns in theory. Sometimes, overlapping peaks prevent the identification of all peaks of the minor rotamer, and on occasion, of the major rotamer. In this document, only ¹H NMR peaks of the major rotamer are reported in the best effort of resolving the peaks.

Cyclosporin analogs demonstrate more complicated conformational flexibility. In CD₃OD, most compounds exist as >6 conformational isomers (Ko, S. Y.; Dalvit, C. Int. J. Pept. Protein Res. 1992, 40, 380-382). In CDCl₃ the spectra are generally less complicated, and for certain compounds, only two conformational isomers are observed. For these compounds the ¹H NMR spectra in CDCl₃ are resolvable, and peaks belonging to the major conformation are reported.

Mini-workup: When a mini-workup (A/B) is indicated in the procedure, it was performed as follows: an aliquot (5 μL) of the reaction mixture was retrieved with a glass pipet and added to a plastic vial containing 0.2 mL organic solvent A and 0.2 mL aqueous solution B. The vial was shaken vigorously and allowed to stand until the two layers partitioned. The organic layer was then used for TLC or LC-MS analysis as specified in the procedure.

Monitoring Reaction Progress by LC-MS: When analysis of the reaction mixture is indicated in the procedure, it was performed as follows. An aliquot (1 μL) of the reaction mixture (or the organic phase of a mini-workup mixture) was diluted with 100 μL 1:1 acetonitrile:water. 1 μL of the diluted solution was injected onto a Waters Acquity UPLC BEH C18 1.7 μm column and eluted with a linear gradient of 5-95% acetonitrile/water (+0.1% formic acid) over 3.0 min. Chromatograms were recorded with a UV detector set at 254 nm and a time-of-flight mass spectrometer (Waters Xevo G2-XS).

General Experimental Procedures: All reactions were performed in oven-dried glassware fitted with rubber septa under a positive pressure of argon, unless otherwise noted. Air- and moisture-sensitive liquids were transferred via syringe or stainless steel cannula. Solutions were concentrated by rotary evaporation at or below 40° C. Analytical thin-layer chromatography (TLC) was performed using glass plates pre-coated with silica gel (0.25-mm, 60-Å pore size, 230-400 mesh, Merck KGA) impregnated with a fluorescent indicator (254 nm). TLC plates were visualized by exposure to ultraviolet light (UV), then were stained by submersion in a 10% solution of phosphomolybdic acid (PMA) in ethanol or an acidic ethanolic solution of p-anisaldehyde, followed by brief heating on a hot plate. The acidic ethanolic solution of p-anisaldehyde solution was prepared by sequential additions of concentrated sulfuric acid (5.0 mL), glacial acetic acid (1.5 mL) and p-anisaldehyde (3.7 mL) to absolute ethanol (135 mL) at 23° C. with efficient stirring. Flash column chromatography was performed with Teledyne ISCO CombiFlash EZ Prep chromatography system, employing pre-packed silica gel cartridges (Teledyne ISCO RediSep).

Solvents and Reagents: Anhydrous solvents were purchased from Acros Organics. Except for those specified in the Starting Materials section, all chemical reagents were purchased from Sigma-Aldrich and AK Scientific. Commercial solvents and reagents were used as received.

Starting Materials: SLF was purchased from Cayman Chemical and/or synthesized following the synthetic route reported by Holt el al. 3′-desamino-3′-hydroxy SLF was synthesized following the synthetic route reported by Holt et al. (Holt, D. A. et al. J. Am. Chem. Soc. 1993, 115, 9925-9938). Cyclosporin A and FK506 were purchased from LC Laboratories. Sorafenib acid [4-(4-(3-(4-chloro-3-(trifluoromethyl)phenyl)ureido)phenoxy)picolinic acid] was purchased from BOC Sciences. Des(hydroxyethyl)dasatinib [N-(2-chloro-6-methylphenyl)-2-((2-methyl-6-(piperazin-1-yl)pyrimidin-4-yl)amino)thiazole-5-carboxamide] was purchased from 5A Chemicals. Lapatinib aldehyde [5-(4-((3-chloro-4-((3-fluorobenzyl)oxy)phenyl)amino)quinazolin-6-yl)furan-2-carbaldehyde] was purchased from AK Scientific. Desmethoxychloro erlotinib [6-(2-chloroethoxy)-N-(3-ethynylphenyl)-7-(2-methoxyethoxy)quinazolin-4-amine] was purchased from AstaTech. Desmethoxychloro gefitinib [N-(3-chloro-4-fluorophenyl)-6-(3-chloropropoxy)-7-methoxyquinazolin-4-amine] was purchased from AstaTech. Tert-butyl 4-(7-bromo-6-chloro-2-((3-ethoxy-3-oxopropyl)amino)-8-fluoroquinazolin-4-yl)piperazine-1-carboxylate was purchased from Pharmaron Inc.

Instrumentation: Proton nuclear magnetic resonance (¹H NMR) spectra and carbon nuclear magnetic resonance (¹³C NMR) spectra were recorded on Bruker AvanceIII HD 2-channel instrument (400 MHz/100 MHz) at 23° C. Proton chemical shifts are expressed in parts per million (ppm, δ scale) and are referenced to residual protium in the NMR solvent (CHCl₃: δ 7.26, D₂HCOD: δ 3.31). Carbon chemical shifts are expressed in parts per million (ppm, δ scale) and are referenced to the carbon resonance of the NMR solvent (CDCl₃: δ 77.0, CD₃OD: δ 49.0). Data are represented as follows: chemical shift, multiplicity (s=singlet, d=doublet, t=triplet, q=quartet, dd=doublet of doublets, dt=doublet of triplets, m=multiplet, br=broad, app=apparent), integration, and coupling constant (J) in Hertz (Hz). High-resolution mass spectra were obtained using a Waters Xevo G2-XS time-of-flight mass spectrometer. Unless otherwise specified, diastereomeric ratios of products are reported as (major diastereomer):(sum of minor diastereomers).

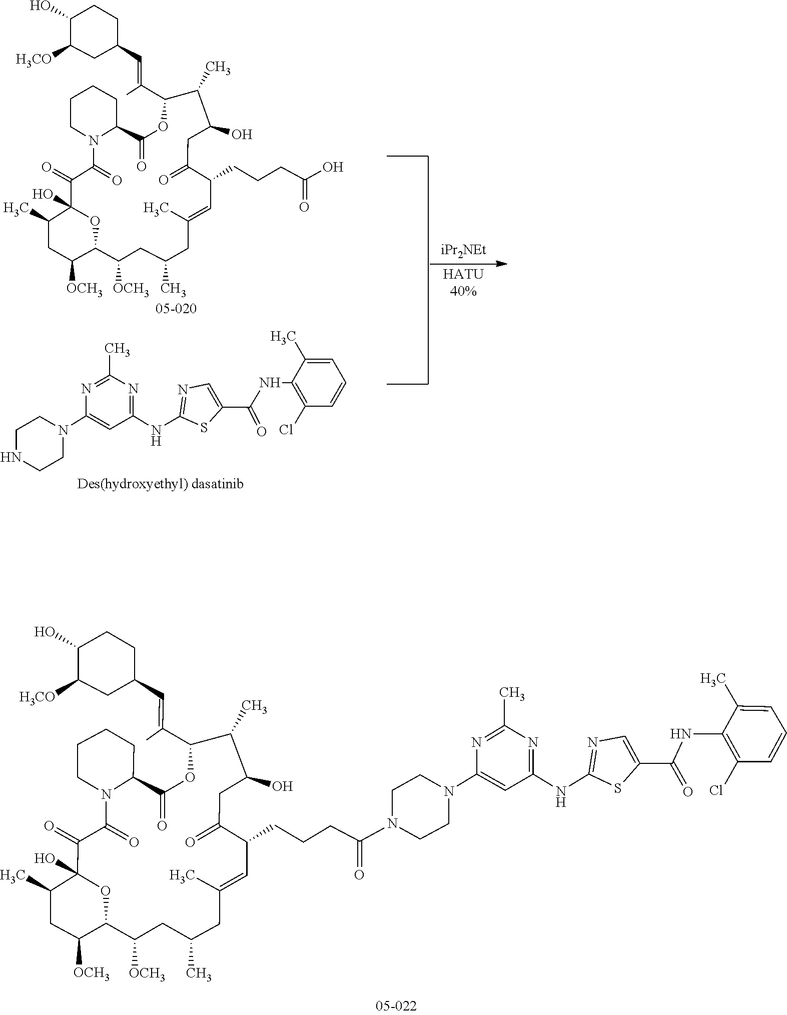 [SMILES: "iPr2NEt" ; CO[C@H]1C[C@@H](C)C/C(C)=C/[C@@H](CCCC(=O)O)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; "HATU40%" ; Cc1nc(Nc2ncc(C(=O)Nc3c(C)cccc3Cl)s2)cc(N2CCNCC2)n1 ; CO[C@H]1C[C@@H](C)C/C(C)=C/[C@@H](CCCC(=O)N2CCN(c3cc(Nc4ncc(C(=O)Nc5c(C)cccc5Cl)s4)nc(C)n3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

An oven-dried 1-dram vial was charged with 05-020 (10 mg, 0.012 mmol), des(hydroxyethyl)dasatinib (5.8 mg, 0.012 mmol), DMF (0.20 mL) and a magnetic stir bar. The solution was cooled to 0° C., then N,N-diisopropylethylamine (6.2 μL, 0.035 mmol) and HATU (5.4 mg, 0.010 mmol) were added sequentially. The resulting mixture was stirred at 0° C. and the reaction progress was monitored by LC-MS. In 30 min, LC-MS analysis indicated that the starting material had been fully consumed. The reaction mixture was diluted with 50% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (5.9 mg, 40%). 3:2 mixture of rotamers. ¹H NMR (400 MHz, Chloroform-d) δ 8.19 (s, 1H), 7.99 (br s, 1H), 7.42-7.31 (m, 2H), 7.26-7.10 (m, 2H), 5.89 (s, 1H), 5.39 (s, 1H), 5.16-5.06 (m, 1H), 4.66 (d, J=5.4 Hz, 1H), 4.48 (d, J=13.8 Hz, 1H), 3.99-3.94 (m, 1H), 3.81-3.68 (m, 5H), 3.68-3.53 (m, 5H), 3.44 (s, 3H), 3.41 (s, 3H), 3.46-3.33 (m, 3H), 3.32 (s, 3H), 3.10-2.98 (m, 3H), 2.79 (d, J=14.7 Hz, 1H), 2.55 (s, 3H), 2.43-2.38 (m, 1H), 2.37 (s, 3H), 2.35-2.25 (m, 2H), 2.24-1.98 (m, 5H), 1.97-1.72 (m, 6H), 1.71-1.62 (m, 6H), 1.62-1.32 (m, 8H), 1.12-1.04 (m, 2H), 1.02 (d, J=6.2 Hz, 3H), 0.95 (d, J=5.6 Hz, 3H), 0.89 (d, J=7.2 Hz, 3H). HRMS (ESI): Calcd for (C₆₅H₉₁ClN₈O₁₄S+H)⁺: 1275.6142, Found: 1275.6085.

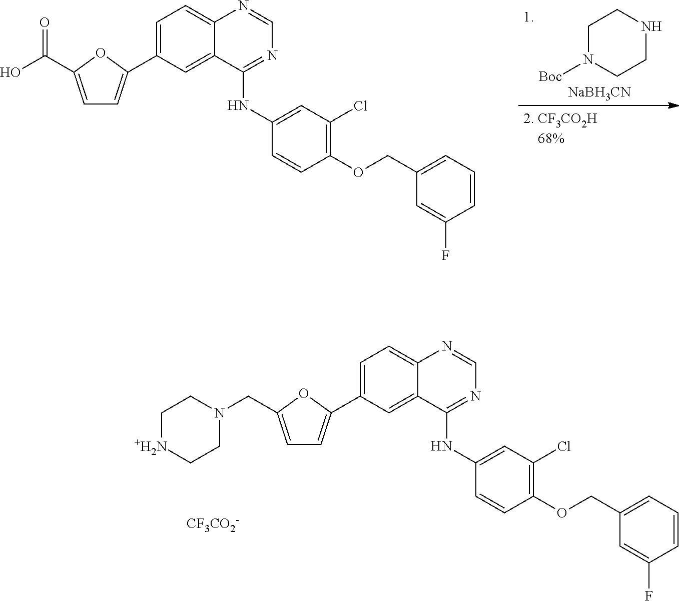

A suspension of 5-[4-[3-chloro-4-[(3-fluorophenyl)methoxy]anilino]quinazolin-6-yl]furan-2-carbaldehyde (100 mg, 0.211 mmol) in 9:1 methanol (1.8 mL):acetic acid (0.2 mL) was sonicated until a fine suspension was formed. 1-Boc-Piperazine (79 mg, 0.42 mmol) was added and the resulting mixture was stirred at 23° C. for 30 min. Sodium cyanoborohydride (20 mg, 0.32 mmol) was added in a single portion at 23° C. The precipitates dissolved over time to a point with a few speckles left in 1 h. At this point TLC analysis (100% ethyl acetate) showed full consumption of the aldehyde starting material. The reaction mixture was concentrated under reduced pressure. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (20-100% ethyl acetate-hexanes) to afford the product as a yellow powder. The yellow powder was resuspended in dichloromethane (2.0 mL), and trifluoroacetic acid (2.0 mL) was added dropwise, giving rise to a bright yellow solution. After standing at 23° C. for 1 h, the solution was concentrated under reduced pressure to afford the product as a yellow powder (93 mg, 68%). HRMS (ESI): Calcd for (C₃₀H₂₇ClFN₅O₂+H)⁺: 544.1910, Found: 544.1882.

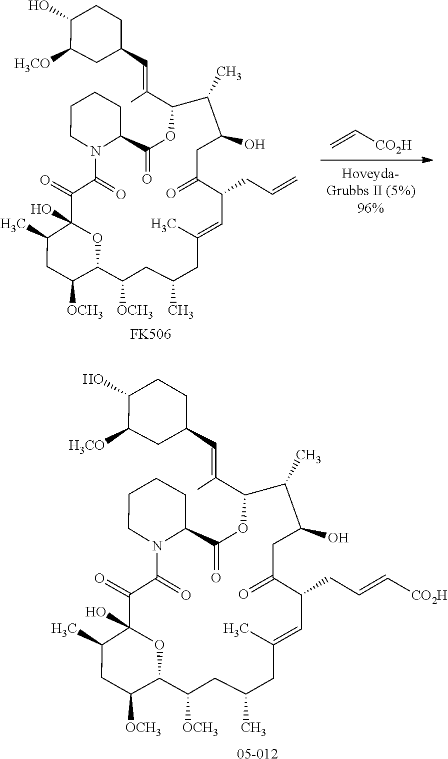

A flame-dried 10-mL microwave vial was flushed with dry argon, and then was charged with FK506 (100 mg, 0.120 mmol), DCE (1.20 mL), and a magnetic stir bar. Acrylic acid (170 mg, 2.49 mmol) and Grubbs Catalyst 2nd Gen (5.3 mg, 0.010 mmol) were added sequentially. The vial was flushed with argon again and sealed with a rubber cap. The reaction mixture was heated at 85° C. for 1 h in a CEM Discover SP microwave reactor. After cooling to 23° C., TLC analysis (100% ethyl acetate) of the reaction mixture showed full disappearance of the starting material and formation of a highly polar new spot. The reaction solution was concentrated in vacuo. The residue was purified by column chromatography (20-100% ethyl acetate-hexanes, 12-g RediSep(R) Rf column, Teledyne ISCO, Lincoln, Nebr.) to afford the product as a yellow powder (101 mg, 96%). NMR: 3:2 mixture of rotamers. ¹H NMR (400 MHz, Chloroform-d) δ 7.01-6.88 (m, 1H), 5.86 (d, J=15.5 Hz, 1H), 5.34 (s, 1H), 5.11 (app t, J=8.8 Hz, 1H), 5.03 (app d, J=8.4 Hz, 1H), 4.68 (d, J=5.2 Hz, 1H), 4.46 (d, J=13.9 Hz, 1H), 3.96-3.84 (m, 1H), 3.72 (d, J=9.3 Hz, 1H), 3.68-3.54 (m, 1H), 3.55-3.41 (m, 3H), 3.43 (s, 3H), 3.41 (s, 3H), 3.32 (s, 3H), 3.08-2.96 (m, 3H), 2.86-2.78 (m, 1H), 2.74-2.62 (m, 1H), 2.49-2.26 (m, 3H), 2.24-2.08 (m, 3H), 2.06-1.98 (m, 2H), 1.98-1.86 (m, 2H), 1.86-1.71 (m, 4H), 1.71-1.60 (m, 6H), 1.59-1.32 (m, 8H), 1.14-1.05 (m, 2H), 1.03 (d, J=6.3 Hz, 3H), 0.96 (d, J=6.4 Hz, 3H), 0.90 (d, J=7.1 Hz, 3H). HRMS (ESI): Calcd for (C₄₅H₆₉NO₁₄−H)⁻: 846.4640, Found: 846.4601.

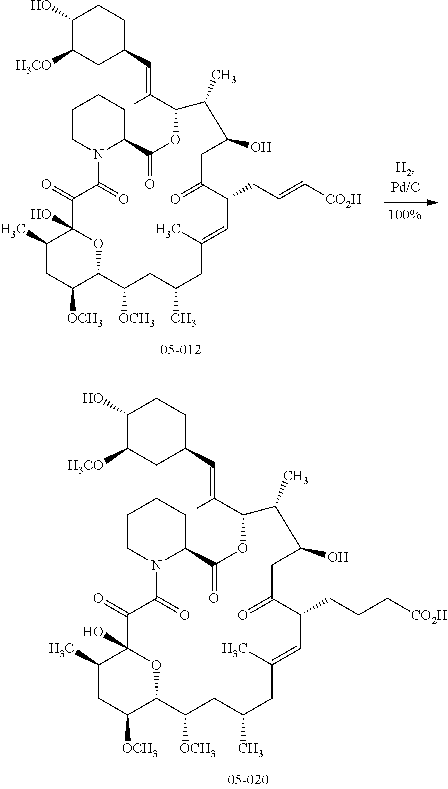 [SMILES: CO[C@H]1C[C@@H](C)C/C(C)=C/[C@@H](C/C=C/C(=O)O)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; CO[C@H]1C[C@@H](C)C/C(C)=C/[C@@H](CCCC(=O)O)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

10 wt % Palladium on carbon (13 mg, 0.010 mmol) was added to a solution of 05-012 (50 mg, 0.060 mmol) in methanol (5 mL) at 23° C. under an atmosphere of argon. The reaction flask was evacuated until effervescence occurred, then flushed with hydrogen gas. The process was repeated three times. The resulting suspension was stirred at 23° C. for 16 h under an atmosphere of hydrogen. The reaction flask was purged with argon, and the reaction suspension was filtered through a pad of Celite. The filter cake was rinsed with ethyl acetate (20 mL). The combined filtrate was concentrated in vacuo, and the residue was purified by column chromatography (0-10% methanol-dichloromethane+0.1% acetic acid) to afford the product as a white solid (50 mg, 100%). 3:2 mixture of rotamers. ¹H NMR (400 MHz, Chloroform-d) δ 5.35-5.31 (m, 1H), 5.13-5.07 (m, 1H), 5.03 (d, J=10.3 Hz, 1H), 4.61 (d, J=5.4 Hz, 1H), 4.43 (d, J=13.8 Hz, 1H), 4.01-3.89 (m, 1H), 3.70 (d, J=9.6 Hz, 1H), 3.64-3.52 (m, 1H), 3.42 (s, 3H), 3.40 (s, 3H), 3.43-3.36 (m, 3H), 3.31 (s, 3H), 3.10-2.93 (m, 3H), 2.79 (dd, J=15.9, 3.0 Hz, 1H), 2.45-2.25 (m, 5H), 2.26-2.10 (m, 3H), 2.09-1.88 (m, 6H), 1.89-1.71 (m, 6H), 1.60 (s, 6H), 1.60-1.35 (m, 8H), 1.13-1.03 (m, 2H), 1.00 (d, J=6.3 Hz, 3H), 0.94 (d, J=6.4 Hz, 3H), 0.88 (d, J=7.1 Hz, 3H). HRMS (ESI): Calcd for (C₄₅H₇₁NO₁₄−H)⁻: 848.4796, Found: 848.4809.

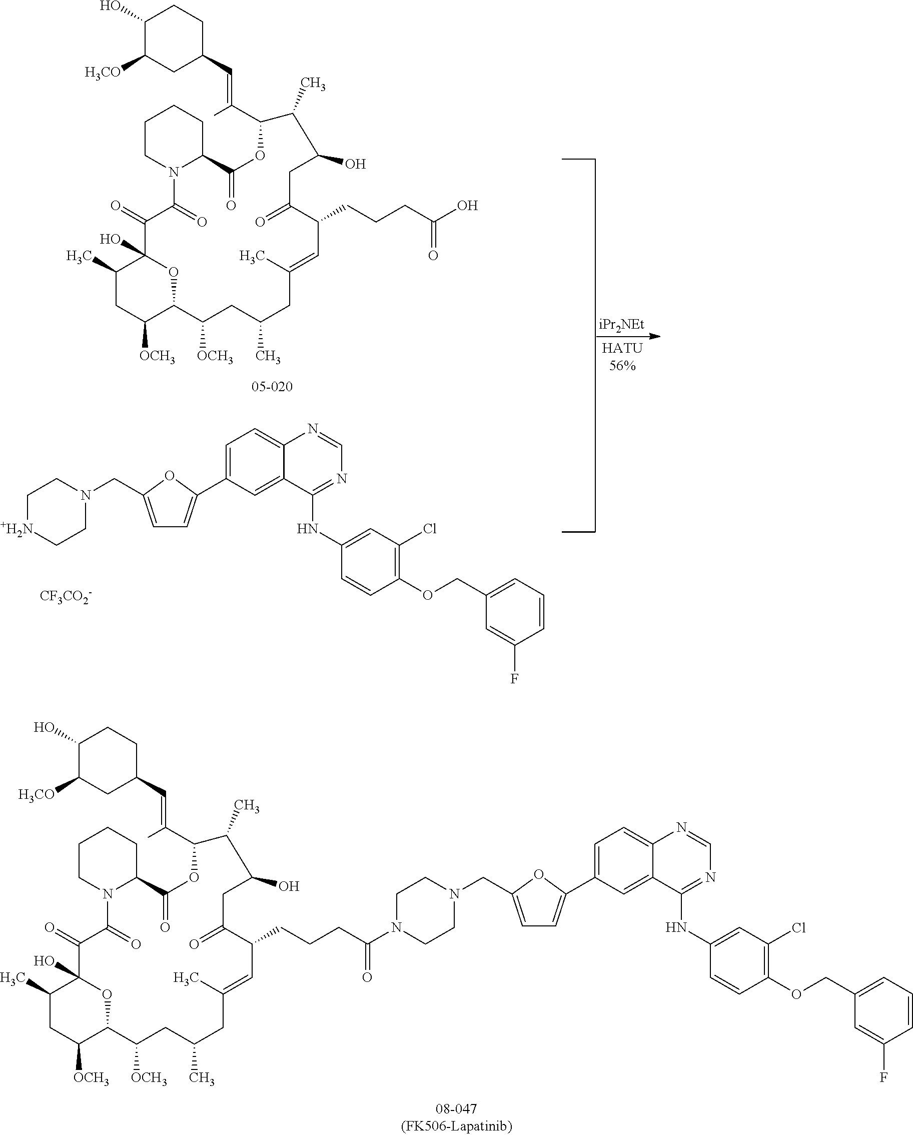

N,N-Diisopropylethylamine (12.3 μL, 0.071 mmol) and HATU (9.8 mg, 0.026 mmol) were added sequentially to a stirred solution of 05-020 (20 mg, 0.024 mmol) and Lapatinib-piperidine (17 mg, 0.026 mmol) in 9:1 dichloromethane (0.9 mL):DMF (0.1 mL). The resulting yellow solution was stirred at 23° C. for 1 h. At this point, LC-MS analysis showed full consumption of the acid starting material and formation of a new specie with the desired m/z. The reaction mixture was concentrated under reduced pressure to remove dichloromethane. The residue was diluted with 50% acetonitrile-water to a volume of 4.1 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a yellow solid (18.7 mg, 56%). 3:2 mixture of rotamers. ¹H NMR (400 MHz, Chloroform-d) δ 8.71 (s, 1H), 8.07-7.94 (m, 2H), 7.90 (d, J=8.7 Hz, 2H), 7.72 (d, J=9.2 Hz, 1H), 7.43-7.34 (m, 1H), 7.27-7.19 (m, 3H), 7.09-6.97 (m, 2H), 6.76 (d, J=3.2 Hz, 1H), 6.55 (s, 1H), 5.42 (s, 1H), 5.18 (d, 3H), 5.12-5.04 (m, 2H), 4.70 (d, J=4.7 Hz, 1H), 4.43 (d, J=13.4 Hz, 1H), 4.07-3.92 (m, 2H), 3.92-3.80 (m, 1H), 3.80-3.66 (m, 2H), 3.61 (d, J=10.1 Hz, 1H), 3.52 (s, 2H), 3.43 (s, 3H), 3.41 (s, 3H), 3.45-3.36 (m, 3H), 3.32 (s, 3H), 3.09-2.95 (m, 2H), 2.95-2.69 (m, 2H), 2.44-2.22 (m, 3H), 2.22-2.09 (m, 3H), 2.05-1.70 (m, 8H), 1.69-1.60 (m, 6H), 1.62-1.32 (m, 8H), 1.11-1.04 (m, 2H), 1.02 (d, J=6.3 Hz, 3H), 0.96 (d, J=6.3 Hz, 3H), 0.86 (d, J=7.3 Hz, 3H). HRMS (ESI): Calcd for (C₇₅H₉₆ClFN₆O₅+2H)^{2+: 688.3381}, Found: 688.3373.

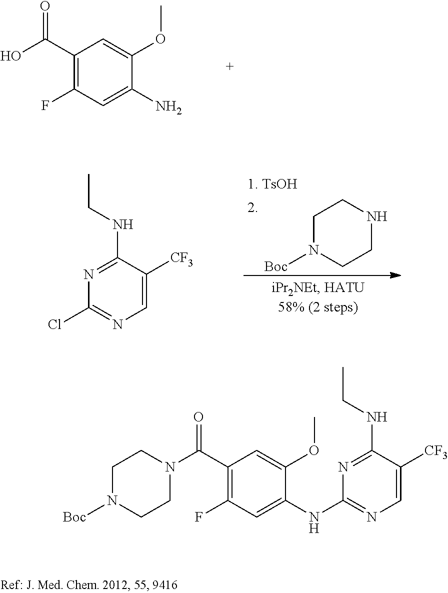 [SMILES: COc1cc(C(=O)O)c(F)cc1N ; CCNc1nc(Cl)ncc1C(F)(F)F ; CC(C)(C)OC(=O)N1CCNCC1 ; CCNc1nc(Nc2cc(F)c(C(=O)N3CCN(C(=O)OC(C)(C)C)CC3)cc2OC)ncc1C(F)(F)F]

A 20-mL vial was charged with 4-amino-2-fluoro-5-methoxy-benzoic acid (100 mg, 0.54 mmol), 2-chloro-N-ethyl-5-(trifluoromethyl)pyrimidin-4-amine (146 mg, 0.650 mmol), p-toluenesulfonic acid monohydrate (51 mg, 0.27 mmol) and 1,4-dioxane (8.1 mL). The mixture was heated to 100° C. with constant stirring. Despite heating not all the solids dissolved. After 2 h, LC-MS analysis showed full conversion to the desired product. The reaction mixture was then cooled to room temperature. The insoluble solids were collected by filtration, washed with 1,4-dioxane (100 mL) and ethyl (50 mL), and air-dried for 12 h to afford the product as a white solid. The crude material was used in the next step without further purification. Dichloromethane (2.67 mL) and N,N-diisopropylamine (93 μL, 0.53 mmol) were added to the crude product from the last reaction. The resulting suspension was cooled to 0° C., and HATU (308 mg, 0.802 mmol) was added in one portion. The mixture was stirred at 0° C. for 15 min before warming to 23° C. and stirring for another 45 min. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (20-50% ethyl acetate-hexanes, 4-g RediSep(R) Rf column, Teledyne ISCO, Lincoln, Nebr.) to afford the product as a white powder (151 mg, 58% over 2 steps). ¹H NMR (400 MHz, Chloroform-d) δ 8.41 (br s, 1H), 8.18 (s, 1H), 6.91 (d, J=6.0 Hz, 1H), 3.92 (s, 3H), 3.76 (s, 2H), 3.68-3.58 (m, 2H), 3.58-3.47 (m, 2H), 3.49-3.35 (m, 4H), 1.48 (s, 9H), 1.34 (t, J=7.2 Hz, 3H). HRMS (ESI): Calcd for (C₂₄H₃₀F₄N₆O₄+H)⁺: 543.2343, Found: 543.2389.

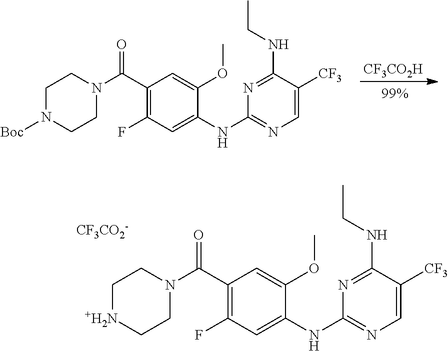 [SMILES: CCNc1nc(Nc2cc(F)c(C(=O)N3CCN(C(=O)OC(C)(C)C)CC3)cc2OC)ncc1C(F)(F)F ; "CF3CO2H" ; "CF3CO2-" ; CCNc1nc(Nc2cc(F)c(C(=O)N3CC[NH2+]CC3)cc2OC)ncc1C(F)(F)F]

Trifluoroacetic acid (0.50 mL) was added dropwise to a solution of tert-butyl 4-[4-[[4-(ethylamino)-5-(trifluoromethyl)pyrimidin-2-yl]amino]-2-fluoro-5-methoxy-benzoyl]piperazine-1-carboxylate (151 mg, 0.28 mmol) in dichloromethane (0.50 mL) at 23° C. and the resulting solution was allowed to stand at 23° C. for 1 h. The reaction mixture was concentrated in vacuo to afford the product as a white solid. To assist removal of residual trifluoroacetic acid, the solids were triturated with ether (10 mL), and the supernatant was removed. The resulting solids were dried under vacuum over night to afford the product as a white powder (153 mg, 99%). ¹H NMR (400 MHz, Methanol-d₄) δ 8.38 (d, J=11.9 Hz, 1H), 8.29 (d, J=1.1 Hz, 1H), 7.14 (d, J=6.1 Hz, 1H), 4.08-4.01 (m, 2H), 4.00 (s, 3H), 3.78-3.69 (m, 2H), 3.66 (q, J=7.2 Hz, 2H), 3.41-3.25 (m, 4H), 1.32 (t, J=7.1 Hz, 3H). HRMS (ESI): Calcd for (C₁₉H₂₁F₄N₆O₂+H)⁺: 443.1813, Found: 443.1786.

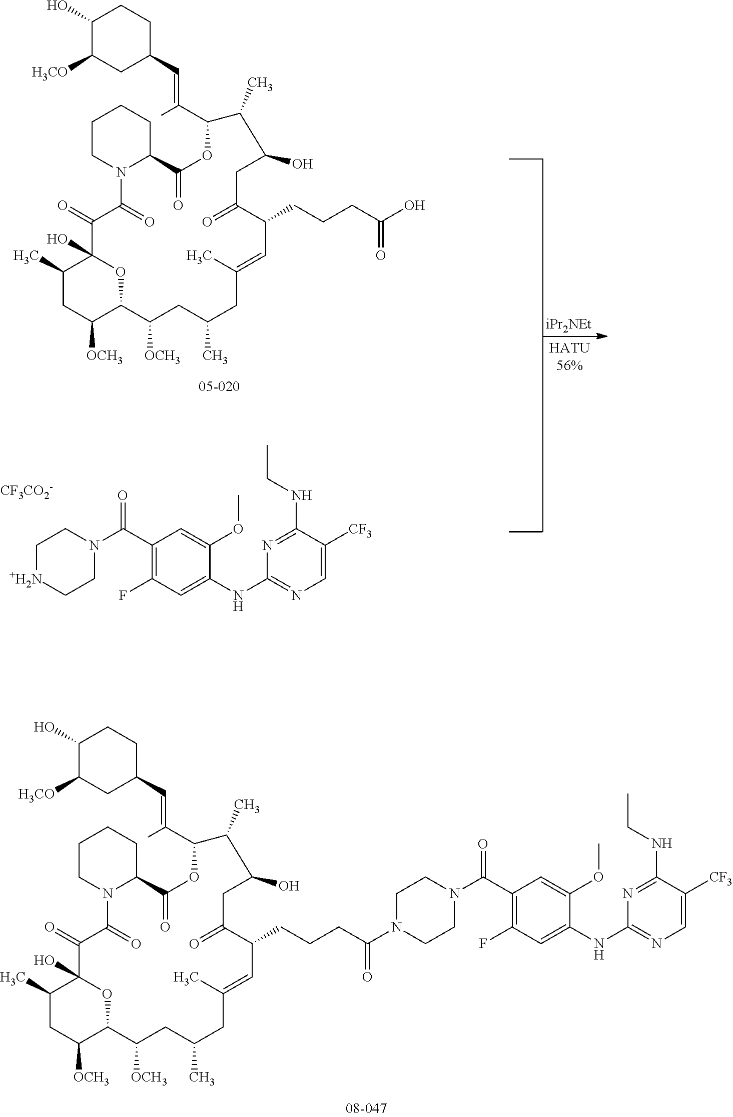

An oven-dried 1-dram vial was charged with 05-020 (20 mg, 0.024 mmol), Pip-GNE7915 (13 mg, 0.023 mmol), DMF (0.12 mL) and a magnetic stir bar. N,N-Diisopropylethylamine (12 μL, 0.071 mmol) was added and the mixture was stirred until all reactants had dissolved. HATU (11 mg, 0.030 mmol) was added as a 10% (w/v) solution in DMF (110 μL), and the reaction progress was monitored by LC-MS. In 30 min, LC-MS analysis showed that the FK506-acid starting material had been fully consumed and a new product with desired m/z had formed. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-10% methanol-dichloromethane, 4-g RediSep(R) Rf column, Teledyne ISCO, Lincoln, Nebr.) to afford the product as a white powder (16.7 mg, 56%). 3:2 mixture of rotamers. ¹H NMR (400 MHz, Chloroform-d) δ 8.45 (d, J=12.4 Hz, 1H), 8.20 (s, 1H), 7.87 (s, 1H), 6.91 (s, 1H), 5.34 (s, 1H), 5.26-5.16 (m, 1H), 5.16-4.92 (m, 2H), 4.61 (d, J=5.4 Hz, 1H), 4.42 (d, J=13.4 Hz, 1H), 3.92 (s, 3H), 3.85-3.65 (m, 5H), 3.65-3.51 (m, 5H), 3.51-3.43 (m, 3H), 3.40 (s, 3H), 3.38 (s, 3H), 3.43-3.31 (m, 3H), 3.29 (s, 3H), 3.24-3.19 (m, 1H), 3.19-3.12 (m, 1H), 3.05-2.96 (m, 2H), 2.79 (dd, J=15.9, 2.4 Hz, 1H), 2.67 (br s, 1H), 2.43-2.22 (m, 5H), 2.22-1.94 (m, 5H), 1.94-1.69 (m, 6H), 1.69-1.60 (m, 6H), 1.60-1.40 (m, 8H), 1.33 (t, J=7.2 Hz, 3H), 1.10-1.02 (m, 2H), 0.99 (d, J=6.3 Hz, 3H), 0.93 (d, J=5.9 Hz, 3H), 0.85 (d, J=7.6 Hz, 3H). HRMS (ESI): Calcd for (C₆₄H₉₁F₄N₇O₁₅+H)⁺: 1274.6587, Found: 1274.6560.

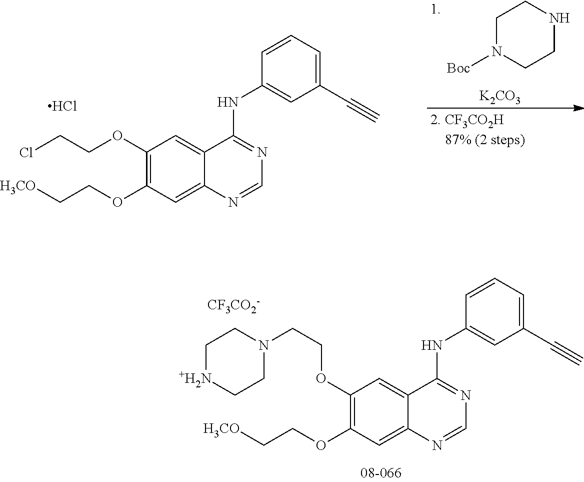

An oven-dried 1-dram vial was charged with 6-(2-chloroethoxy)-N-(3-ethynylphenyl)-7-(2-methoxyethoxy)quinazolin-4-amine hydrochloride (100 mg, 0.23 mmol), 1-Boc-piperazine (86 mg, 0.46 mmol), Potassium carbonate (95 mg, 0.69 mmol). DMF (1.15 mL) was added via syringe. The vial was flushed with argon and closed with a rubber septum fitted with a needle connected to an argon source. The mixture was warmed to 80° C. In 1 h, LC-MS analysis showed consumption of the starting material and formation of one single product with the desired m/z. The reaction mixture was partitioned between ethyl acetate (5 mL) and water (5 mL), and the layers were separated. The aqueous layer was extracted with ethyl acetate (3×5 mL). The combined organic layers were dried over sodium sulfate, and the dried solution was concentrated. The residue was purified by column chromatography (0-10% methanol-dichloromethane) to afford the product as a brown solid (112 mg, 88%). tert-butyl 4-[2-[4-(3-ethynylanilino)-7-(2-methoxyethoxy)quinazolin-6-yl]oxyethyl]piperazine-1-carboxylate (112 mg, 0.20 mmol) was dissolved in 1:1 dichloromethane (0.50 mL):Trifluroacetic Acid (0.50 mL) at 23° C. and the resulting solution was allowed to stand at 23° C. for 1 h. At this point, LC-MS analysis showed full consumption of the starting material and formation of one single product with desired m/z. The reaction mixture was concentrated in vacuo to give a syrup, which was triturated with ether (10 mL) to afford the product as a white powder (113 mg, 98%). ¹H NMR (400 MHz, Methanol-d₄) δ 8.69 (s, 1H), 8.01 (s, 1H), 7.84 (t, J=1.7 Hz, 1H), 7.70 (dt, J=7.7, 1.9 Hz, 1H), 7.49-7.36 (m, 2H), 7.26 (s, 1H), 4.45 (t, J=4.9 Hz, 2H), 4.41-4.33 (m, 2H), 3.88-3.79 (m, 2H), 3.57 (s, 1H), 3.43 (s, 3H), 3.37-3.29 (m, 4H), 3.22 (t, J=4.9 Hz, 2H), 3.17 (d, J=6.8, 3.8 Hz, 4H). HRMS (ESI): Calcd for (C₂₅H₂₉N₅O₃+H)⁺: 448.2343, Found: 448.2350.

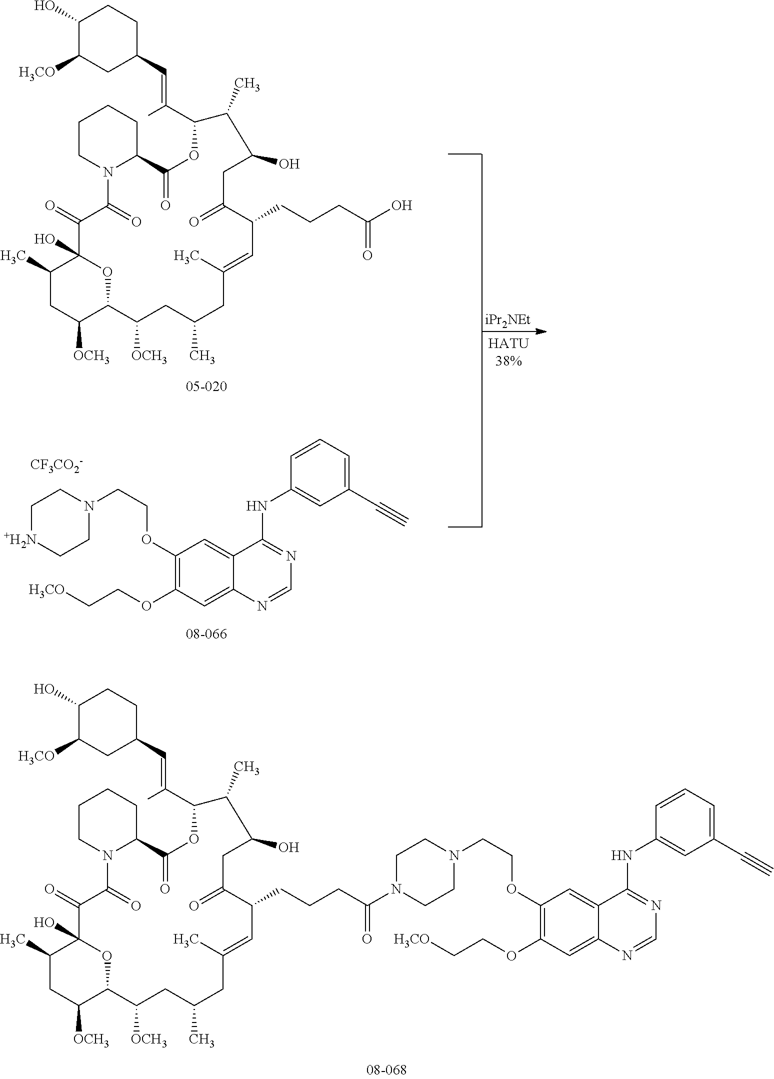

A 1-dram vial was charged with 05-020 (20 mg, 0.024 mmol), Pip-Erlotinib (13 mg, 0.024 mmol), DMF (0.12 mL) and a magnetic stir bar. N,N-Diisopropylethylamine (12 μL, 0.071 mmol) was added and the mixture was stirred until all reactants had gone into solution. HATU (10.7 mg, 0.0282 mmol) was added as a freshly made 10% w/v solution in DMF. The mixture was stirred at 23° C. while the reaction progress was monitored by LC-MS. In a total of 1 h, LC-MS analysis showed full consumption of the starting material. The reaction mixture was partitioned between ethyl acetate (5 mL) and water (5 mL), and the layers were separated. The aqueous layer was extracted with ethyl acetate (2×5 mL). The combined organic layers were dried over sodium sulfate, and the dried solution was concentrated. The residue was purified by column chromatography (0-10% methanol-dichloromethane) to afford the product as a pale-yellow solid (11.5 mg, 38%). 3:2 mixture of rotamers. ¹H NMR (400 MHz, Chloroform-d) δ 8.64 (s, 1H), 7.87 (dt, J=8.4, 1.4 Hz, 1H), 7.81-7.74 (m, 1H), 7.40-7.30 (m, 2H), 7.25-7.23 (m, 2H), 5.37 (s, 1H), 5.09-4.98 (m, 2H), 4.68 (d, J=4.8 Hz, 1H), 4.38 (d, J=12.9 Hz, 1H), 4.31-4.24 (m, 4H), 4.05-3.90 (m, 1H), 3.90-3.80 (m, 4H), 3.66-3.50 (m, 2H), 3.46 (s, 3H), 3.39 (s, 3H), 3.38 (s, 3H), 3.38-3.33 (m, 3H), 3.29 (s, 3H), 3.09 (s, 1H), 3.05-2.93 (m, 2H), 2.80-2.53 (m, 4H), 2.37-2.20 (m, 4H), 2.19-2.06 (m, 4H), 2.06-1.83 (m, 4H), 1.83-1.51 (m, 8H), 1.49-1.28 (m, 14H), 1.10-1.02 (m, 2H), 0.98 (d, J=6.2 Hz, 3H), 0.92 (d, J=5.6 Hz, 3H), 0.85 (d, J=7.2 Hz, 3H). HRMS (ESI): Calcd for (C₇₀H₉₈N₆O₁₆+H)⁻: 1279.7117, Found: 1279.7131.

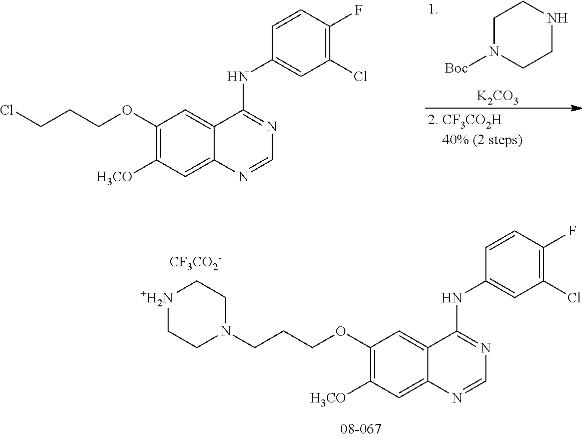

An oven-dried 1-dram vial was charged with N-(3-chloro-4-fluoro-phenyl)-6-(3-chloropropoxy)-7-methoxy-quinazolin-4-amine (100 mg, 0.25 mmol), 1-Boc-piperazine (94 mg, 0.50 mmol), Potassium carbonate (105 mg, 0.76 mmol) and a magnetic stir bar. DMF (2.00 mL) was added via syringe. The vial was flushed with argon and closed with a rubber septum fitted with a needle connected to an argon source. The mixture was warmed to 80° C. In 1 h, LC-MS analysis showed consumption of the starting material and formation of one single product with the desired m/z. The reaction mixture was partitioned between ethyl acetate (5 mL) and water (5 mL), and the layers were separated. The aqueous layer was extracted with ethyl acetate (3×5 mL). The combined organic layers were dried over sodium sulfate, and the dried solution was concentrated. The residue was purified by column chromatography (0-10% methanol-dichloromethane) to afford the product as a white solid (57 mg, 41%). tert-butyl 4-[3-[4-(3-chloro-4-fluoro-anilino)-7-methoxy-quinazolin-6-yl]oxypropyl]piperazine-1-carboxylate (57 mg, 0.104 mmol) was dissolved in 1:1 dichloromethane (0.50 mL):trifluroacetic acid (0.50 mL), and the resulting solution was allowed to stand at 23° C. for 1 h. The solution was concentrated in vacuo to afford the product as a brown solid (58 mg, 99%). ¹H NMR (400 MHz, Methanol-d₄) δ 8.76 (s, 1H), 8.00 (s, 1H), 7.95 (dd, J=6.6, 2.6 Hz, 1H), 7.68 (ddd, J=8.9, 4.2, 2.6 Hz, 1H), 7.40 (t, J=8.9 Hz, 1H), 7.28 (s, 1H), 4.38 (t, J=5.7 Hz, 2H), 4.11 (s, 3H), 3.50 (t, J=5.3 Hz, 4H), 3.32-3.28 (m, 4H, this peak is covered by the CD₂HOD solvent peak), 3.20 (t, J=7.1 Hz, 2H), 2.34 (p, J=6.4 Hz, 2H). HRMS (ESI): Calcd for (C₂₂H₂₅ClN₅O₂+H)⁺: 446.1754, Found: 446.1748.

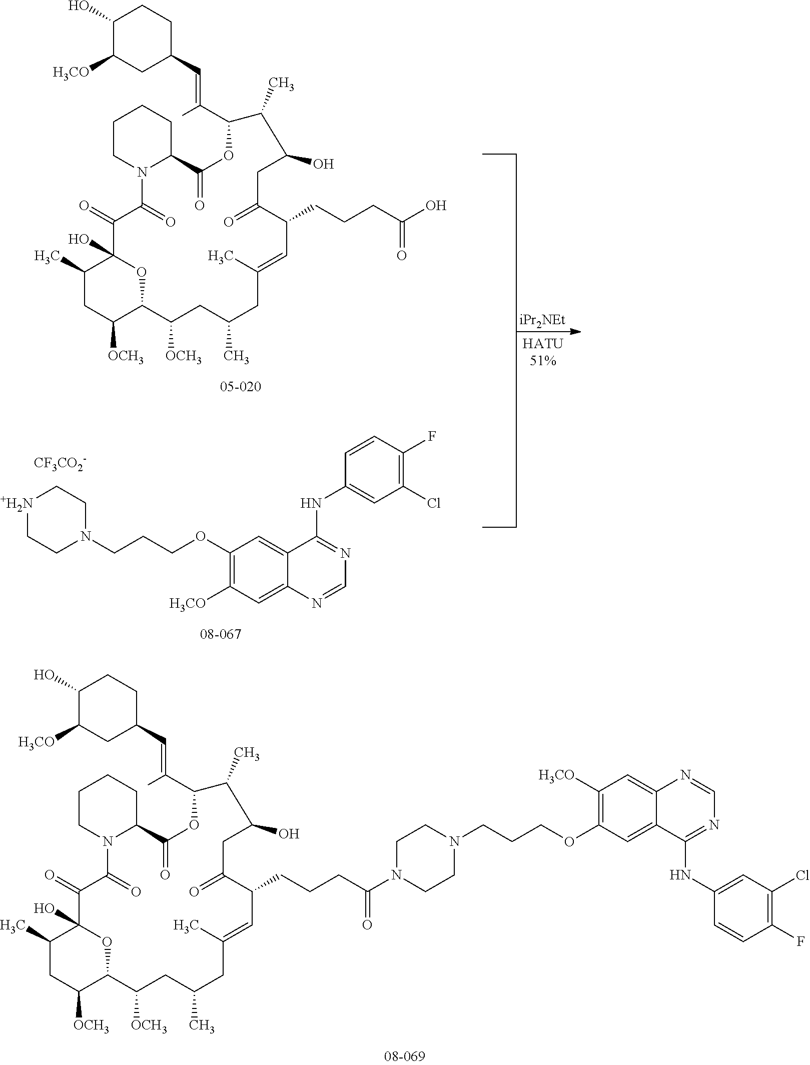

A 1-dram vial was charged with 05-020 (20 mg, 0.024 mmol), Pip-gefetinib (13 mg, 0.024 mmol), DMF (0.12 mL) and a magnetic stir bar. N,N-Diisopropylethylamine (12 μL, 0.071 mmol) was added and the mixture was stirred until all reactants had gone into solution. HATU (10.7 mg, 0.0282 mmol) was added as a 10% w/v solution in DMF. The resulting solution was stirred at 23° C. and the reaction progress was monitored by LC-MS. In 15 min, LC-MS showed ˜80% consumption of the amine starting material. Additional HATU (1.07 mg, as 10% solution in DMF) was added. In a total of 1 h, LC-MS analysis showed full consumption of the starting material. The reaction mixture was partitioned between ethyl acetate (5 mL) and water (5 mL), and the layers were separated. The aqueous layer was extracted with ethyl acetate (2×5 mL). The combined organic layers were dried over sodium sulfate, and the dried solution was concentrated. The residue was purified by column chromatography (0-10% methanol-dichloromethane) to afford the product as a pale-yellow solid (15.5 mg, 51%). 3:2 mixture of rotamers. ¹H NMR (400 MHz, Chloroform-d) δ 8.63 (s, 1H), 8.03-7.87 (m, 1H), 7.70 (d, J=5.7 Hz, 1H), 7.50-7.38 (m, 1H), 7.24 (s, 1H), 7.20-7.12 (m, 2H), 5.40 (s, 1H), 5.21-5.02 (m, 3H), 4.73 (s, 1H), 4.39 (d, J=13.3 Hz, 1H), 4.29-4.14 (m, 4H), 4.14-4.03 (m, 2H), 3.99 (s, 3H), 3.83 (d, J=9.7 Hz, 1H), 3.80-3.56 (m, 4H), 3.51-3.47 (m, 3H), 3.42 (s, 3H), 3.39 (s, 3H), 3.35 (br s, 3H), 3.22 (s, 2H), 3.08-2.71 (m, 6H), 2.40-2.11 (m, 5H), 2.11-1.90 (m, 5H), 1.87-1.65 (m, 6H), 1.62-1.53 (m, 6H), 1.53-1.32 (m, 8H), 1.06-1.01 (m, 2H), 0.96 (d, J=6.0 Hz, 3H), 0.91 (d, J=6.6 Hz, 3H), 0.87 (d, J=6.5 Hz, 3H). HRMS (ESI): Calcd for (C₆₇H₉₄ClFN₆O₁₅+H)⁺: 1277.6528, Found: 1277.6564.

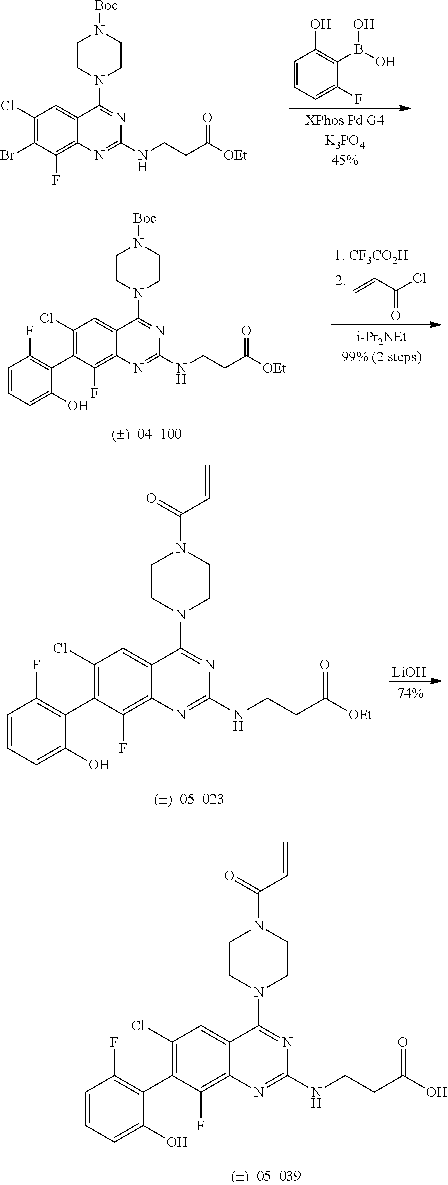

A 20-mL vial was charged with (2-fluoro-6-hydroxy-phenyl)boronic acid (278 mg, 1.78 mmol), tert-butyl 4-[7-bromo-6-chloro-2-[(3-ethoxy-3-oxo-propyl)amino]-8-fluoro-quinazolin-4-yl]piperazine-1-carboxylate (200 mg, 0.36 mmol), XPhos Pd G4 precatalyst (14.2 mg, 0.018 mmol), and 1:1 THF:water (4.0 mL). Argon was bubbled through the mixture for 5 min, then the vial was closed with a rubber septum fitted with a needle connected to an argon source. An 0.5 M aqueous solution of potassium phosphate (0.91 mL, 2.14 mmol) was added dropwise via syringe. After 16 h, both LC-MS and TLC analysis showed only ˜50% conversion. Additional catalyst (14.2 mg) was added and the mixture was stirred at 50° C. for another 2 h. However, no further progress was detected after this second portion of catalyst. The reaction mixture was partitioned between ethyl acetate (10 mL) and 10% citric acid (10 mL). The layers were separated, and the aqueous layer was extracted with ethyl acetate (2×10 ml). The combined organic layers were dried over sodium sulfate, and the dried solution was concentrated. The residue was purified by column chromatography (20-50% ethyl acetate-hexanes, 4-g RediSep(R) Rf column, Teledyne ISCO, Lincoln, Nebr.) to afford the product as a yellow powder (97 mg, 46%) and recovered bromide starting material (72 mg, 36%). ¹H NMR (400 MHz, Chloroform-d) δ 7.39 (s, 1H), 7.34-7.24 (m, 1H), 6.85 (d, J=8.3 Hz, 1H), 6.70 (t, J=8.5 Hz, 1H), 5.66 (s, 1H), 4.16 (q, J=7.1 Hz, 2H), 3.74 (app q, J=6.6 Hz, 2H), 3.71-3.51 (m, 8H), 2.65 (td, J=6.5, 1.4 Hz, 2H), 1.50 (s, 9H), 1.26 (t, J=7.1 Hz, 3H). HRMS (ESI): Calcd for (C₂₈H₃₂ClF₂N₅O₅+H)⁺: 592.2138, Found: 592.2148.

tert-butyl 4-[6-chloro-2-[(3-ethoxy-3-oxo-propyl)amino]-8-fluoro-7-(2-fluoro-6-hydroxy-phenyl)quinazolin-4-yl]piperazine-1-carboxylate (97 mg, 0.16 mmol) was dissolved in 1:1 trifluoroacetic acid (0.50 mL):dichloromethane (0.50 mL) and the resulting mixture was allowed to stand at 23° C. for 1 h. The solution was then concentrated under reduced pressure to afford the product as a yellow foam. dichloromethane (1.45 mL) and N,N-diisopropylethylamine (43 μL, 0.25 mmol) were added sequentially to the foam. After stirring for 10 min, the material had fully dissolved. The solution was cooled to −78° C., and acryloyl chloride (14 μL, 0.1700 mmol) was added dropwise via syringe. The resulting solution was warmed to 0° C., and the reaction progress was monitored by LC-MS. In 30 min, LC-MS analysis showed conversion to a single product with the desired m/z. The reaction mixture was partitioned between water (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (20-80% ethyl acetate-hexanes, 4-g RediSep(R) Rf column, Teledyne ISCO, Lincoln, Nebr.) to afford the product as a yellow powder (100 mg, 99%). ¹H NMR (400 MHz, Methanol-d₄) δ 8.05 (s, 1H), 7.70 (dd, J=13.0, 5.9 Hz, 1H), 7.45-7.29 (m, 3H), 6.82 (dt, J=8.4, 0.8 Hz, 1H), 6.80-6.73 (m, 1H), 4.41 (s, 4H), 3.93-3.84 (m, 2H), 3.56-3.49 (m, 6H), 2.74 (t, J=6.3 Hz, 2H), 1.20 (t, J=7.0 Hz, 3H). HRMS (ESI): Calcd for (C₂₆H₂₆ClF₂N₅O₄+H)⁺: 546.1719, Found: 546.1733.

Lithium hydroxide hydrate (1:1:1) (23 mg, 0.55 mmol) was added to a solution of ethyl 3-[[6-chloro-8-fluoro-7-(2-fluoro-6-hydroxy-phenyl)-4-(4-prop-2-enoylpiperazin-1-yl)quinazolin-2-yl]amino]propanoate (100 mg, 0.18 mmol) in 1:1 Water (1.10 mL):THF (1.10 mL). The resulting mixture was stirred at 23° C., and the reaction progress was monitored by LC-MS. In 2 h, The ethyl ester had fully hydrolyzed. The volatile solvents were removed by rotary evaporation. The remaining aqueous suspension was acidified with 10% citric acid (2 mL), and then extracted with ethyl acetate (3×10 mL). The combined organic layers were dried over sodium sulfate, and the dried solution was concentrated to afford the product (71 mg, 74%). ¹H NMR (400 MHz, Methanol-d₄) δ 7.83 (s, 1H), 7.32 (td, J=8.3, 6.7 Hz, 1H), 6.91-6.77 (m, 2H), 6.73 (ddd, J=9.2, 8.3, 1.0 Hz, 1H), 6.29 (dd, J=16.8, 1.9 Hz, 1H), 5.82 (dd, J=10.6, 1.9 Hz, 1H), 4.11-3.84 (m, 8H), 3.78 (t, J=6.5 Hz, 2H), 2.68 (t, J=6.5 Hz, 2H). HRMS (ESI): Calcd for (C₂₄H₂₂ClF₂N₅O₄+H)⁺: 518.1396, Found: 518.1397.

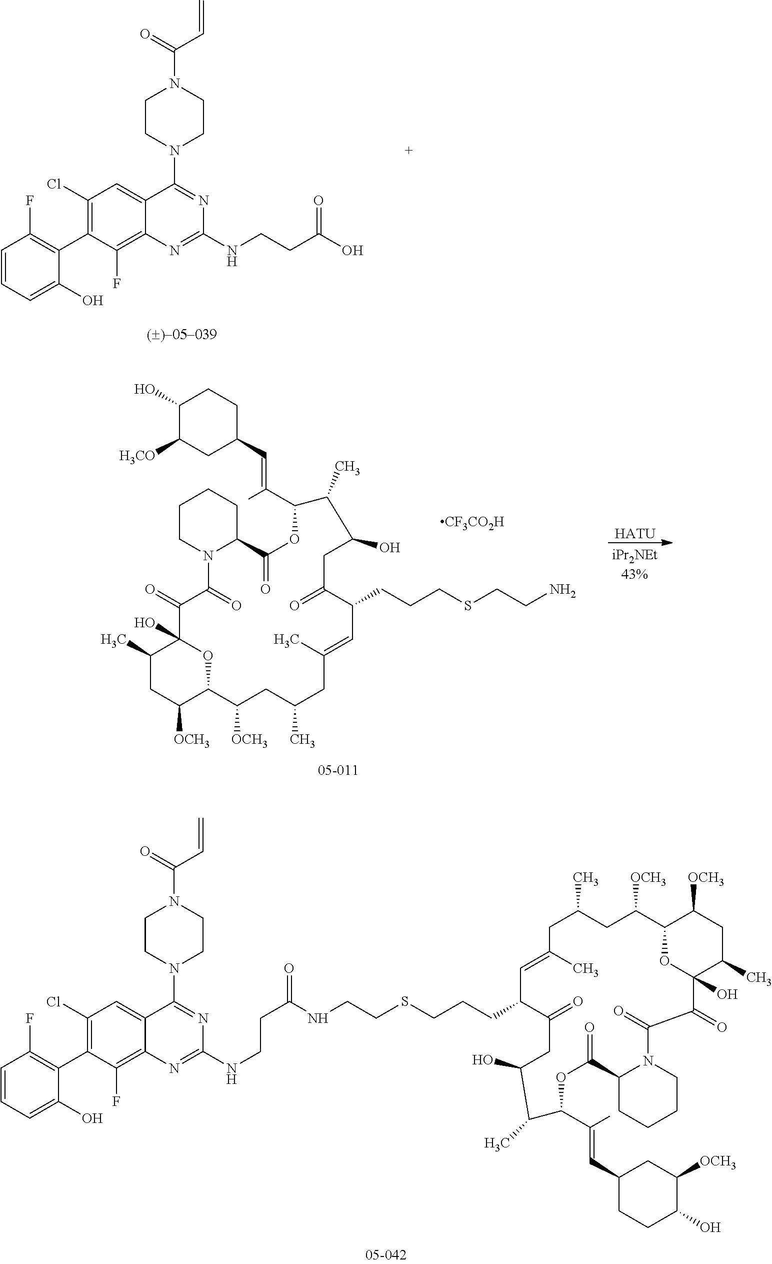 [SMILES: C=CC(=O)N1CCN(c2nc(NCCC(=O)O)nc3c(F)c(-c4c(O)cccc4F)c(Cl)cc23)CC1 ; CO[C@H]1C[C@@H](C)C/C(C)=C/[C@@H](CCCSCCN)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; "·CF3CO2H" ; "HATU" ; C=CC(=O)N1CCN(c2nc(NCCC(=O)NCCSCCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/[C@@H]5CC[C@@H](O)[C@H](OC)C5)[C@H](C)[C@@H](O)CC3=O)[C@H](C)C[C@@H]4OC)nc3c(F)c(-c4c(O)cccc4F)c(Cl)cc23)CC1]

A 1-dram vial was charged with (±)-05-039 (5.0 mg, 0.010 mmol), 05-011 (9.6 mg, 0.010 mmol), and DMF (0.30 mL). N,N-Diisopropylethylamine (5.0 μL, 0.030 mmol) and HATU (4.4 mg, 0.012 mmol) were added sequentially to the solution at 23° C., and the resulting mixture was stirred at 23° C. while the reaction progress was monitored by LC-MS. In 15 min, LC-MS showed full consumption of the amine starting material and formation of a new product with the expected m/z. The reaction mixture was diluted with 50% acetonitrile-water to a volume of 4.7 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (5.7 mg, 43%). 3:2 mixture of rotamers. ¹H NMR (400 MHz, Chloroform-d) δ 8.28 (s, 1H), 7.77-7.62 (m, 1H), 6.90-6.72 (m, 2H), 6.64 (dd, J=16.9, 10.7 Hz, 1H), 6.39 (d, J=16.8 Hz, 1H), 5.80 (d, J=10.3 Hz, 1H), 5.35 (d, J=3.5 Hz, 1H), 5.11-4.96 (m, 2H), 4.61-4.32 (m, 2H), 4.07-3.73 (m, 6H), 3.72-3.53 (m, 2H), 3.42 (s, 3H), 3.42 (s, 3H), 3.41 (s, 3H), 3.40-3.37 (m, 3H), 3.17-2.86 (m, 6H), 2.68-2.52 (m, 4H), 2.49-2.24 (m, 6H), 2.24-1.86 (m, 8H), 1.86-1.70 (m, 6H), 1.70-1.61 (m, 6H), 1.61-1.24 (m, 8H), 1.14-1.05 (m, 2H), 1.05-0.81 (m, 9H). HRMS (ESI): Calcd for (C₇₀H₉₆ClF₂N₇O₁₅S+H)⁺: 1380.6420, Found: 1380.6404.

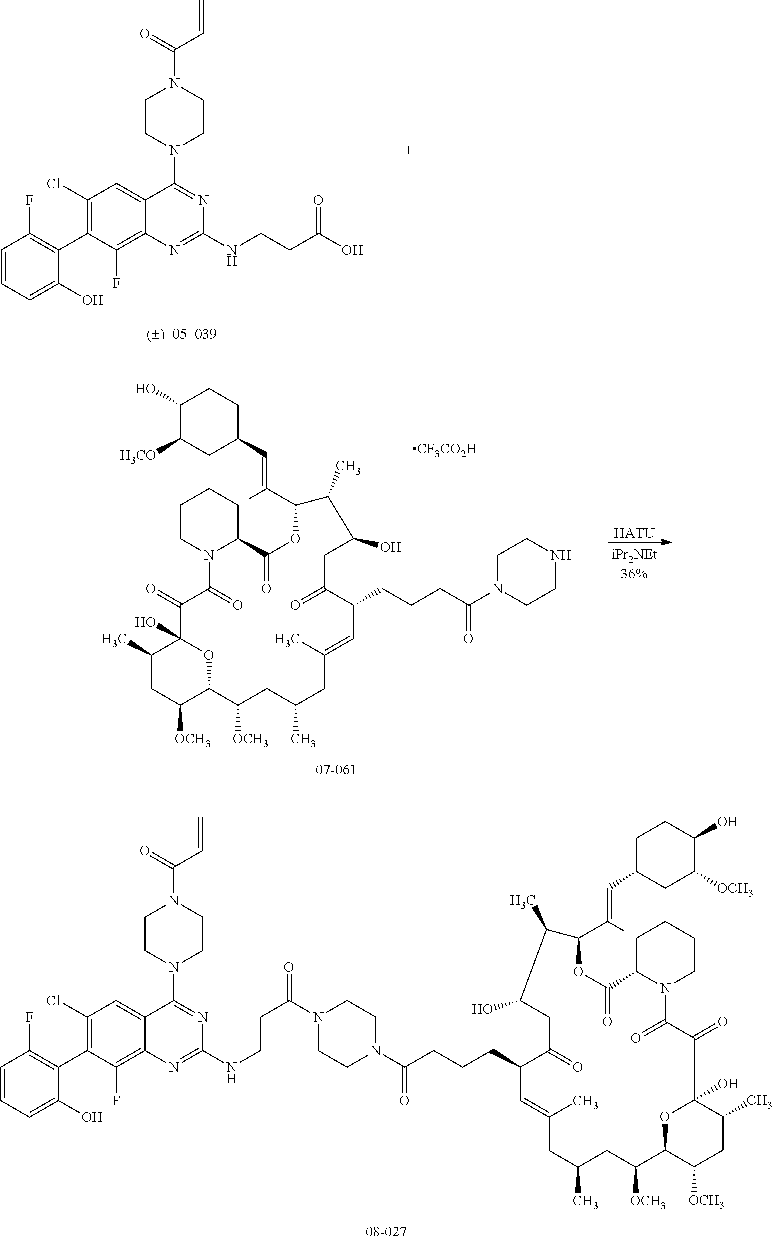

A 1-dram vial was charged with (±)-05-039 (5.5 mg, 0.011 mmol), 08-019 (10.0 mg, 0.010 mmol), and DMF (0.30 mL). N,N-Diisopropylethylamine (5.0 μL, 0.030 mmol) and HATU (7.6 mg, 0.019 mmol) were added sequentially to the solution at 23° C., and the resulting mixture was stirred at 23° C. while the reaction progress was monitored by LC-MS. In 15 min, LC-MS showed full consumption of the amine starting material and formation of a new product with the expected m/z. The reaction mixture was diluted with 50% acetonitrile-water to a volume of 4.2 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (4.9 mg, 36%). 3:2 mixture of rotamers. ¹H NMR (400 MHz, Methanol-d₄) δ 8.09 (s, 1H), 7.46-7.29 (m, 1H), 6.89-6.71 (m, 3H), 6.33 (d, J=16.6 Hz, 1H), 5.85 (d, J=10.6 Hz, 1H), 5.33-5.09 (m, 3H), 4.34 (br s, 4H), 4.06-3.83 (m, 4H), 3.77-3.52 (m, 10H), 3.43 (s, 3H), 3.42 (s, 3H), 3.41-3.38 (m, 2H), 3.35 (s, 3H), 3.09-2.94 (m, 2H), 2.94-2.75 (m, 4H), 2.50-2.27 (m, 6H), 2.27-1.87 (m, 6H), 1.88-1.72 (m, 4H), 1.72-1.52 (m, 6H), 1.52-1.28 (m, 8H), 1.15-1.02 (m, 2H), 1.02-0.86 (m, 9H). HRMS (ESI): Calcd for (C₇₃H₉₉ClF₂N₈O₁₆+H)⁺: 1417.6914, Found: 1417.6904.

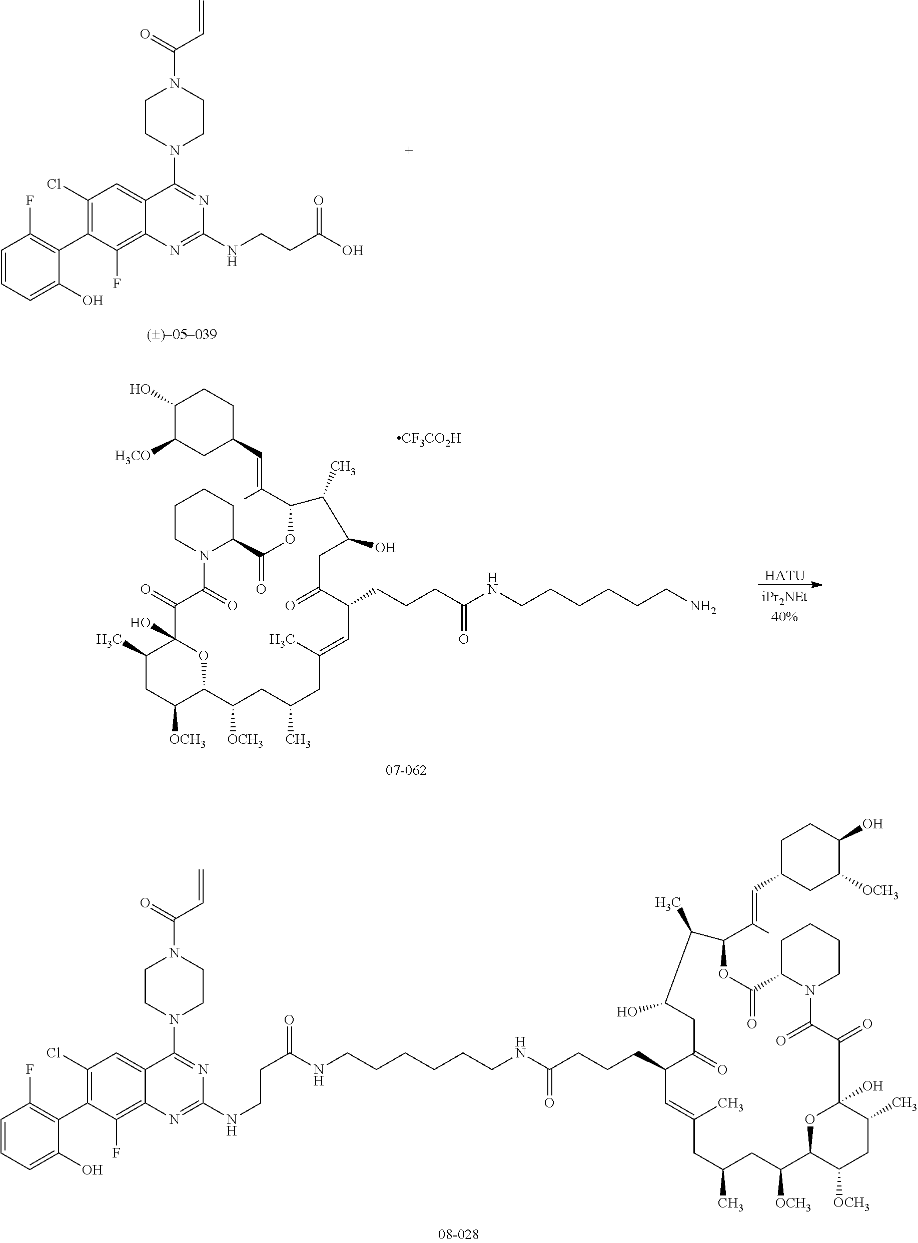

A 1-dram vial was charged with (±)-05-039 (5.5 mg, 0.011 mmol), 08-062 (10.0 mg, 0.010 mmol), and DMF (0.30 mL). N,N-Diisopropylethylamine (5.0 μL, 0.030 mmol) and HATU (7.6 mg, 0.019 mmol) were added sequentially to the solution at 23° C., and the resulting mixture was stirred at 23° C. while the reaction progress was monitored by LC-MS. In 15 min, LC-MS showed full consumption of the amine starting material and formation of a new product with the expected m/z. The reaction mixture was diluted with 50% acetonitrile-water to a volume of 4.2 mL, and the solution was filtered through a 0.45 μM PTFE syringe filter. The filtrate was purified by reverse-phase HPLC (Waters XBridge C₁₈ column 5 μm particle size 30×250 mm, 5-95% acetonitrile-water+0.1% formic acid, 40 min, 20 mL/min) to afford the product as a white solid (5.5 mg, 40%). 3:2 mixture of rotamers. ¹H NMR (400 MHz, Methanol-d₄) δ 7.77 (s, 1H), 7.35-7.26 (m, 1H), 6.89-6.68 (m, 3H), 6.28 (d, J=16.1 Hz, 1H), 5.82 (d, J=12.3 Hz, 1H), 5.28-5.19 (m, 1H), 5.18-4.99 (m, 2H), 4.60 (s, 2H), 4.40-4.26 (m, 1H), 4.17-4.06 (m, 1H), 3.95-3.83 (m, 8H), 3.84-3.74 (m, 2H), 3.56-3.47 (m, 2H), 3.44 (s, 3H), 3.43 (s, 3H), 3.40 (s, 3H), 3.39-3.36 (m, 2H), 3.20-3.02 (m, 4H), 2.61-2.50 (m, 2H), 2.44-2.29 (m, 4H), 2.25-2.09 (m, 4H), 2.09-1.89 (m, 4H), 1.89-1.65 (m, 6H), 1.65-1.33 (m, 10H), 1.31-1.23 (m, 8H), 1.12-1.02 (m, 2H), 1.03-0.78 (m, 9H). HRMS (ESI): Calcd for (C₇₅H₁₀₅ClF₂N₈O₁₆+H)⁺: 1447.7383, Found: 1447.7435.

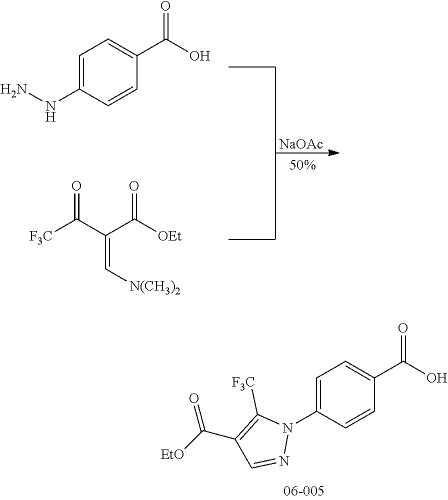 [SMILES: NNc1ccc(C(=O)O)cc1 ; CCOC(=O)/C(=C\N(C)C)C(=O)C(F)(F)F ; CCOC(=O)c1cnn(-c2ccc(C(=O)O)cc2)c1C(F)(F)F ; "NaOAc"]

A 20-mL vial was charged with ethyl (2Z)-2-(dimethylaminomethylene)-4,4,4-trifluoro-3-oxo-butanoate (526 mg, 2.20 mmol), 4-hydrazinobenzoic acid (304 mg, 2.00 mmol), sodium acetate (180 mg, 2.2 mmol) and ethanol (5.0 mL). The mixture was warmed to 70° C. and kept stirred at that temperature. In 4 h, a large amount of precipitation had formed. TLC analysis (50% ethyl acetate-hexanes) showed full conversion of the starting material to a less polar compound. The reaction mixture was filtered through a sintered glass funnel. The collected solids were rinsed with cold ethanol (10 mL) and then ether (10 mL). The combined filtrate was concentrated under reduced pressure. The residue was purified by column chromatography (0-60% ethyl acetate-hexanes+0.1% acetic acid, 4-g RediSep(R) Rf column, Teledyne ISCO, Lincoln, Nebr.) to afford the product as a yellow powder (332 mg, 50%). ¹H NMR (400 MHz, Chloroform-d) δ 8.31-8.22 (m, 2H), 8.16 (d, J=0.7 Hz, 1H), 7.57 (d, J=8.5 Hz, 2H), 4.39 (q, J=7.1 Hz, 2H), 1.40 (t, J=7.2 Hz, 3H). HRMS (ESI): Calcd for (C₁₄H₁₁F₃N₂O₄−H)⁻: 327.0593, Found: 327.0589.

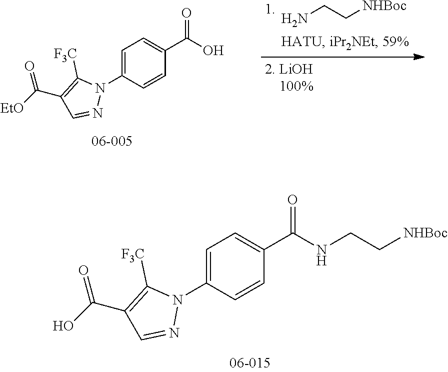 [SMILES: CCOC(=O)c1cnn(-c2ccc(C(=O)O)cc2)c1C(F)(F)F ; CC(C)(C)OC(=O)NCCN ; CC(C)(C)OC(=O)NCCNC(=O)c1ccc(-n2ncc(C(=O)O)c2C(F)(F)F)cc1]

N,N-diisopropylethylamine (0.32 mL, 1.83 mmol) and HATU (278.01 mg, 0.7300 mmol) were added sequentially to a mixed solution of N-tert-Boc-ethylenediamine (117.14 mg, 0.7300 mmol) and 4-[4-ethoxycarbonyl-3-(trifluoromethyl)pyrazol-1-yl]benzoic acid (200 mg, 0.6100 mmol) in 9:1 dichloromethane:DMF (1.2 mL). The resulting mixture was stirred at 23° C. for 30 min, at which point LC-MS analysis showed full consumption of the acid starting material and formation of a slightly less polar compound. The reaction mixture was concentrated under reduced pressure. The residue was purified by column chromatography (20-50% ethyl acetate-hexanes, 4-g RediSep(R) Rf column, Teledyne ISCO, Lincoln, Nebr.) to afford the product as a yellow powder (170 mg, 59%). Lithium hydroxide hydrate (27 mg, 0.64 mmol) was added to a suspension of ethyl 1-[4-[2-(tert-butoxycarbonylamino)ethylcarbamoyl]phenyl]-3-(trifluoromethyl)pyrazole-4-carboxylate (100 mg, 0.21 mmol) in 1:1:1 methanol (0.50 mL):THF (0.50 mL):Water (0.50 mL) at 23° C. The resulting mixture turned into a clear solution immediately. In 30 min, LC-MS analysis showed full conversion to the carboxylic acid. 10% aqueous citric acid (3 mL) was added, and the resulting mixture was extracted with ethyl acetate (3×10 mL). The combined organic layers were dried over sodium sulfate, and the dried solution was concentrated to afford the product as a white powder (99 mg, 100%). ¹H NMR (400 MHz, Chloroform-d) δ 8.21 (s, 1H), 8.01 (d, J=8.3 Hz, 2H), 7.53 (d, J=8.9 Hz, 2H), 3.66-3.58 (m, 2H), 3.54-3.41 (m, 2H), 1.45 (s, 9H). HRMS (ESI): Calcd for (C₁₉H₂₁F₃N₄O₅−H)⁻: 441.1386, Found: 441.1370.

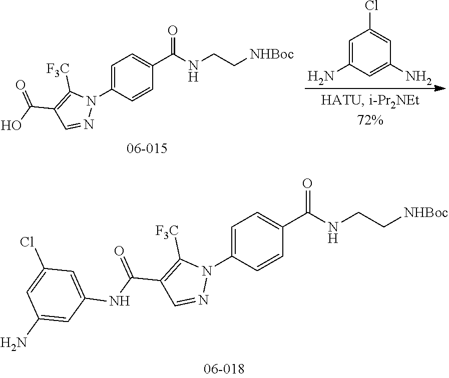 [SMILES: CC(C)(C)OC(=O)NCCNC(=O)c1ccc(-n2ncc(C(=O)O)c2C(F)(F)F)cc1 ; Nc1cc(N)cc(Cl)c1 ; CC(C)(C)OC(=O)NCCNC(=O)c1ccc(-n2ncc(C(=O)Nc3cc(N)cc(Cl)c3)c2C(F)(F)F)cc1]

N,N-Diisopropylamine (87 μL, 0.50 mmol) and HATU (45 mg, 0.12 mmol) were added sequentially to a solution of 1-[4-[2-(tert-butoxycarbonylamino)ethylcarbamoyl]phenyl]-3-(trifluoromethyl)pyrazole-4-carboxylic acid (44 mg, 0.100 mmol) and 5-Chloro-1,3-benzenediamine (57 mg, 0.40 mmol) in 9:1 dichloromethane:DMF (0.5 mL) at 23° C. and the reaction progress was monitored by LC-MS. In 1 h, LC-MS analysis showed full consumption of the acid starting material and formation of a single, desired product. TLC analysis (100% ethyl acetate) showed that the excess diamine was easily separated from the product in this case. The reaction mixture was directly loaded onto a silica gel cartridge (˜2 g). Purification by column chromatography (20-100% ethyl acetate-hexanes, 4-g RediSep Rf Column, Teledyne ISCO, Lincoln, Nebr.) afforded the product as a white solid (51 mg, 72%). ¹H NMR (400 MHz, Methanol-d₄) δ 8.11 (s, 1H), 8.03 (d, J=8.5 Hz, 2H), 7.61 (d, J=8.6 Hz, 2H), 7.02 (s, 1H), 6.98 (s, 1H), 6.52 (t, J=1.9 Hz, 1H), 3.59-3.45 (m, 2H), 3.43-3.28 (m, 2H), 1.43 (s, 9H). HRMS (ESI): Calcd for (C₂₅H₂₇ClF₃N₆O₄+H)⁺: 567.1734, Found: 567.1751.

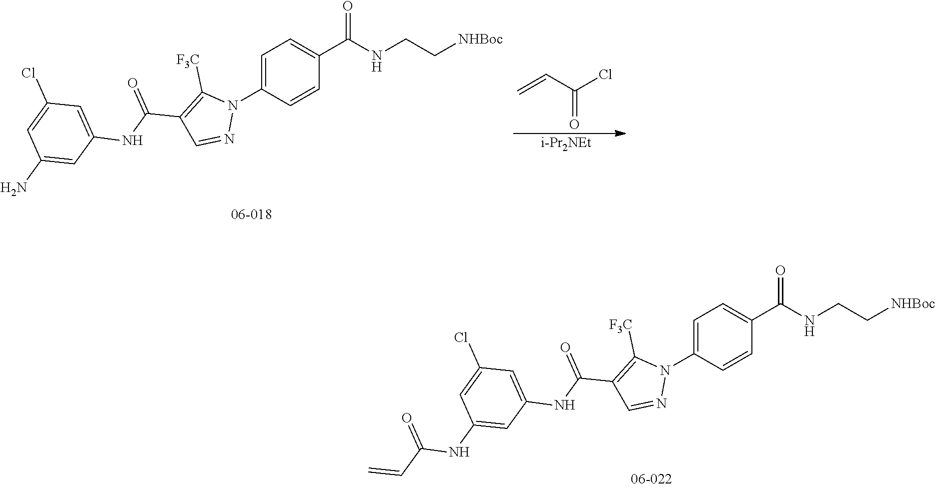

Acryloyl chloride (10.7 μL, 0.13 mmol) was added dropwise to an ice-cold solution of tert-butyl N-[2-[[4-[4-[(3-amino-5-chloro-phenyl)carbamoyl]-5-(trifluoromethyl)pyrazol-1-yl]benzoyl]amino]ethyl]carbamate (51 mg, 0.090 mmol) and triethylamine (37 μL, 0.27 mmol) in dichloromethane (1.0 mL) at 0° C. Upon addition the reaction mixture turned into a polymer-like gel. Nevertheless, TLC analysis (100% ethyl acetate) showed a new UV-active spot being formed. LC-MS analysis of an aliquot of filtered material showed full consumption of the starting material. The reaction mixture was partitioned between saturated aqueous sodium bicarbonate solution (5 mL) and dichloromethane (5 mL). The mixture was filtered to remove the polymeric material. The layers were separated, and the aqueous layer was extracted with dichloromethane (2×25 mL). The combined organic layers were dried over sodium sulfate. The dried solution was filtered, and the filtrate was concentrated. The residue was purified by column chromatography (20-100% ethyl acetate-hexanes, 4-g RediSep(R) Rf column, Teledyne ISCO, Lincoln, Nebr.) to afford the product as a yellow solid (22 mg, 39%). ¹H NMR (400 MHz, Methanol-d₄) δ 8.18 (s, 1H), 8.05 (d, J=8.3 Hz, 2H), 8.02-7.96 (m, 1H), 7.68-7.61 (m, 3H), 7.56 (s, 1H), 6.47-6.40 (m, 2H), 5.82 (dd, J=8.9, 2.9 Hz, 1H), 3.54-3.45 (m, 4H), 1.44 (s, 9H). HRMS (ESI): Calcd for (C₂₈H₂₈ClF₃N₆O₅+H)⁺: 621.1840, Found: 621.1855.

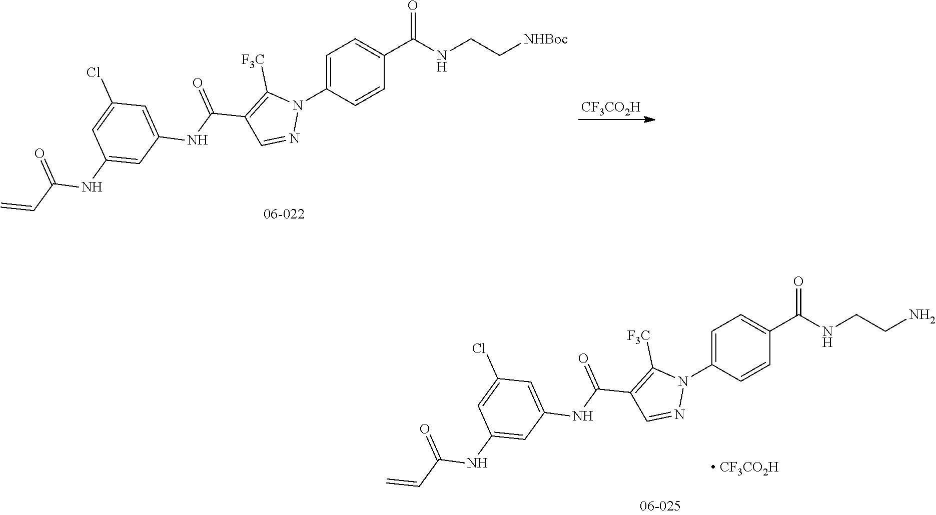 [SMILES: C=CC(=O)Nc1cc(Cl)cc(NC(=O)c2cnn(-c3ccc(C(=O)NCCNC(=O)OC(C)(C)C)cc3)c2C(F)(F)F)c1 ; "CF3CO2H" ; C=CC(=O)Nc1cc(Cl)cc(NC(=O)c2cnn(-c3ccc(C(=O)NCCN)cc3)c2C(F)(F)F)c1 ; "· CF3CO2H"]

tert-butyl N-[2-[[4-[4-[[3-chloro-5-(prop-2-enoylamino)phenyl]carbamoyl]-5-(trifluoromethyl)pyrazol-1-yl]benzoyl]amino]ethyl]carbamate (23 mg, 0.0400 mmol) was dissolved in 50% trifluoroacetic acid-dichloromethane (1.0 mL). The resulting solution was allowed to stand at 23° C. for 1 h. LC-MS analysis at this point showed full convertion of the starting material to a single product. The reaction mixture was directly concentrated to afford the product as a white solid (23 mg, 98%). ¹H NMR (400 MHz, Methanol-d₄) δ 8.22-8.19 (m, 1H), 8.10 (d, J=8.8 Hz, 2H), 8.00 (t, J=1.9 Hz, 1H), 7.68 (d, J=8.5 Hz, 2H), 7.61 (t, J=1.9 Hz, 1H), 7.56 (t, J=1.9 Hz, 1H), 6.46-6.36 (m, 2H), 5.82 (dd, J=9.0, 2.9 Hz, 1H), 3.77-3.69 (m, 2H), 3.22 (t, J=5.9 Hz, 2H). HRMS (ESI): Calcd for (C₂₃H₂₀ClF₃N₆O₃+H)⁺: 521.1316, Found: 521.1319.

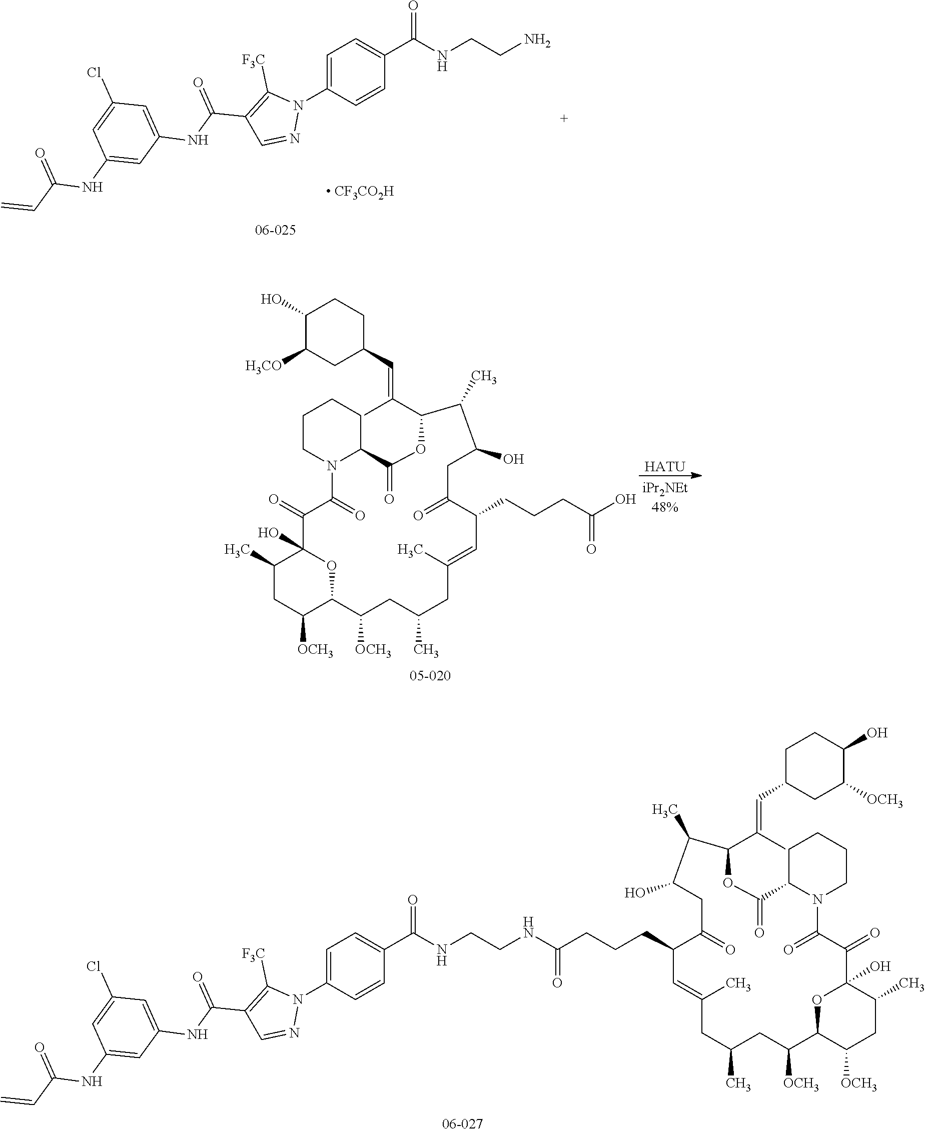

N,N-Diisopropylethylamine (8.2 μL, 0.047 mmol) and HATU (9.0 mg, 0.024 mmol) were added sequentially to a mixed solution of 05-020 (14.7 mg, 0.017 mmol) and 06-025 (10 mg, 0.016 mmol) in DMF (0.20 mL) at 23° C. The resulting mixture was stirred at 23° C., and the reaction progress was monitored by LC-MS. In 1 h, LC-MS analysis showed full consumption of the acid starting material. The residue was diluted with 50% acetonitrile-water to a volume of 3.5 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (10.2 mg, 48%). 3:2 mixture of rotamers. ¹H NMR (400 MHz, Chloroform-d) δ 9.01 (s, 1H), 8.21 (s, 1H), 8.00 (s, 1H), 7.92 (d, J=8.5 Hz, 3H), 7.78 (s, 1H), 7.64 (d, J=13.4 Hz, 2H), 7.59-7.46 (m, 3H), 6.52-6.46 (m, 1H), 6.43 (d, J=17.1 Hz, 1H), 6.28 (dd, J=16.8, 10.2 Hz, 1H), 5.76 (d, J=11.1 Hz, 1H), 5.30 (s, 1H), 5.07-4.94 (m, 2H), 4.63 (d, J=4.3 Hz, 1H), 4.39 (d, J=13.5 Hz, 1H), 3.96-3.88 (m, 2H), 3.68-3.44 (m, 5H), 3.39 (s, 3H), 3.38 (s, 3H), 3.36-3.33 (m, 3H), 3.28 (s, 3H), 3.25-3.16 (m, 2H), 3.06-2.95 (m, 2H), 2.90 (t, J=12.7 Hz, 1H), 2.62 (d, J=15.6 Hz, 1H), 2.39-1.91 (m, 10H), 1.91-1.61 (m, 6H), 1.61-1.55 (m, 6H), 1.56-1.29 (m, 8H), 1.06-1.00 (m, 2H), 0.96 (d, J=6.3 Hz, 3H), 0.91 (d, J=5.7 Hz, 3H), 0.83 (d, J=7.2 Hz, 3H). HRMS (ESI): Calcd for (C₆₈H₈₉ClF₃N₇O₁₆−H)⁻: 1350.5928, Found: 1350.5908.

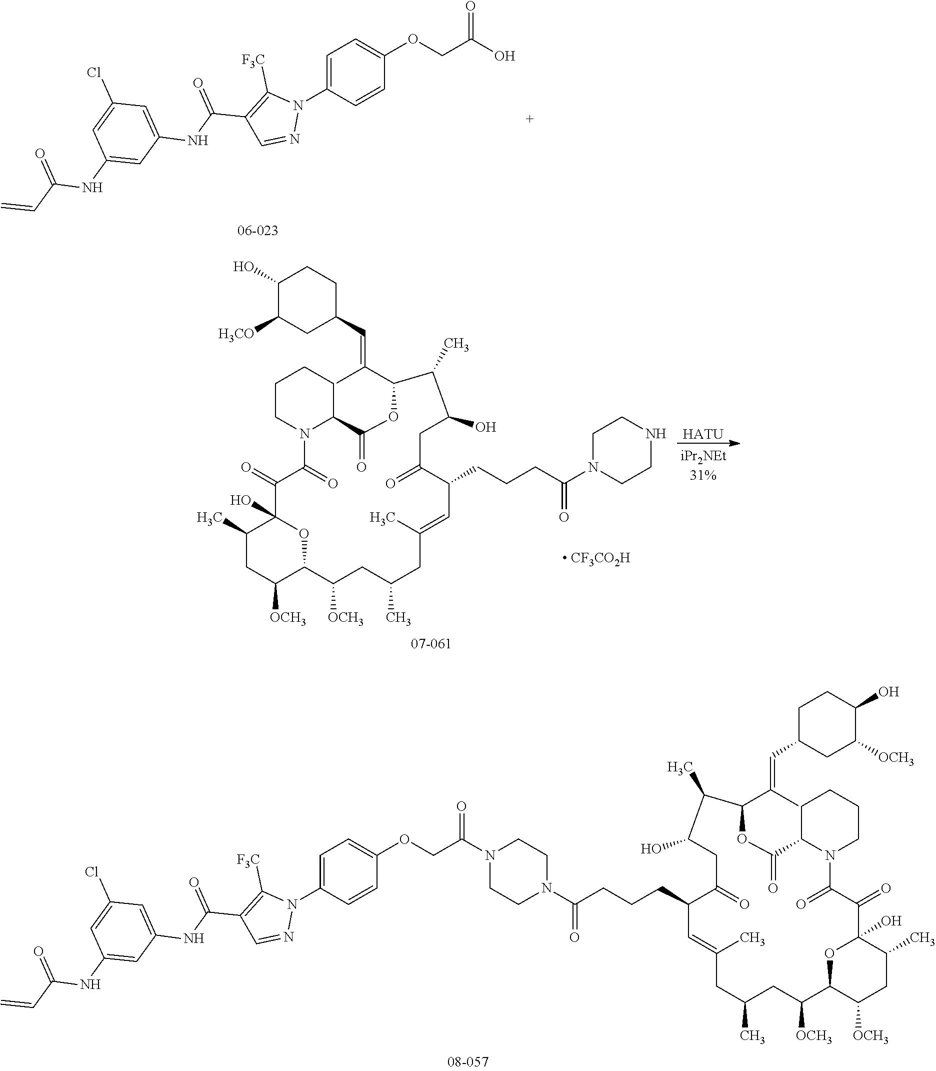

An oven-dried 1-dram vial was charged with 06-023 (5.4 mg, 0.011 mmol) and 07-061 (10 mg, 0.010 mmol), DMF (0.20 mL) and magnetic stir bar. N,N-Diisopropylethylamine (8.4 μL, 0.048 mmol) and HATU (4.4 mg, 0.012 mmol) were added sequentially to the solution, and the mixture was stirred at 23° C. while the reaction progress was monitored by LC-MS. In 1 h, LC-MS analysis showed full consumption of the FK506 starting material. The residue was diluted with 50% acetonitrile-water to a volume of 4.4 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a yellow solid (4.3 mg, 31%). ¹H NMR (400 MHz, Chloroform-d) δ 7.99 (s, 1H), 7.94-7.80 (m, 1H), 7.61-7.47 (m, 2H), 7.43-7.34 (m, 2H), 7.12-7.04 (m, 2H), 6.45 (d, J=16.9 Hz, 1H), 6.29-6.18 (m, 1H), 5.81 (d, J=10.4 Hz, 1H), 5.35 (s, 1H), 5.15-4.94 (m, 2H), 4.84-4.77 (m, 3H), 4.45-4.31 (m, 1H), 3.99-3.77 (m, 2H), 3.72-3.53 (m, 10H), 3.41 (s, 3H), 3.37 (s, 3H), 3.35-3.31 (m, 3H), 3.30 (s, 3H), 3.05-2.97 (m, 2H), 2.84-2.63 (m, 1H), 2.42-2.20 (m, 5H), 2.01 (d, 5H), 1.67 (s, 6H), 1.61-1.52 (m, 6H), 1.51-1.26 (m, 8H), 1.10-1.01 (m, 2H), 1.00 (d, J=6.3 Hz, 3H), 0.93 (d, J=6.8 Hz, 3H), 0.85 (d, J=7.1 Hz, 3H). HRMS (ESI): Calcd for (C₇₁H₉₃ClF₃N₇O₁₇+H)⁺: 1408.6347, Found: 1408.6415.

Cyclosporin Derivatives

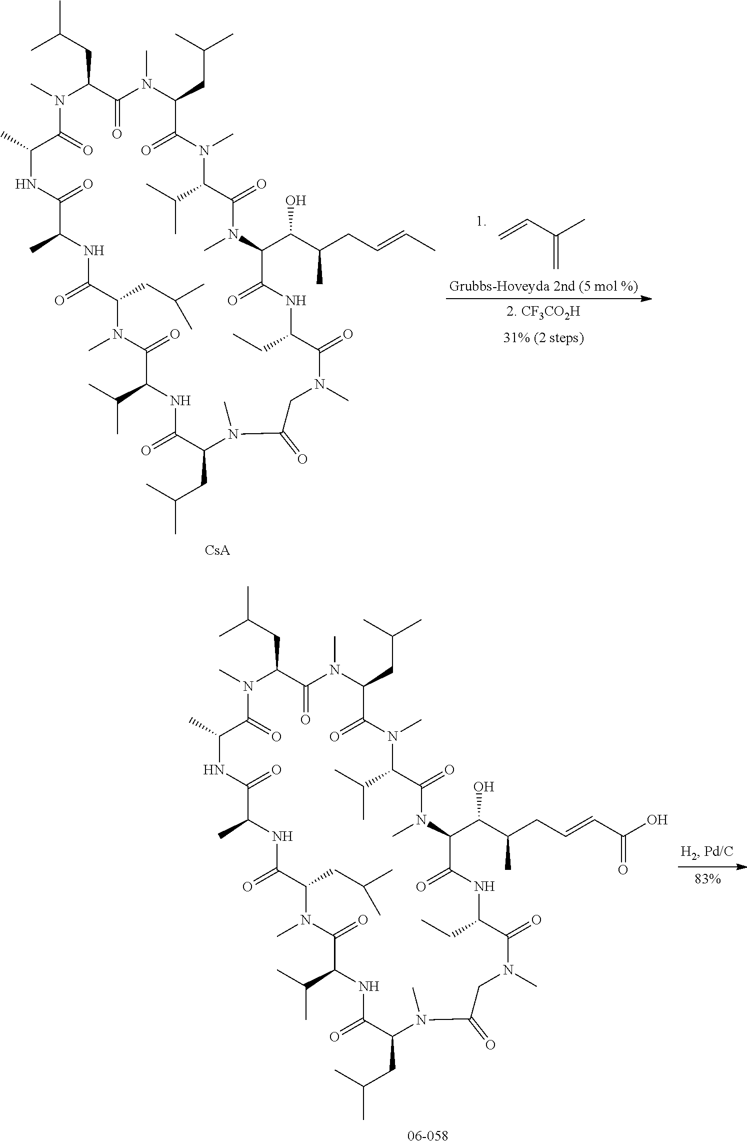

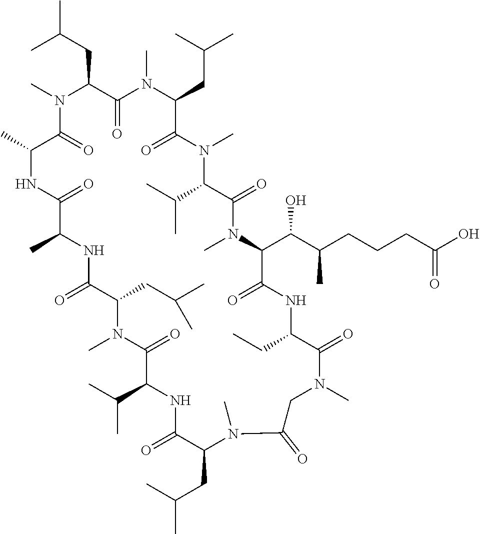 [SMILES: CC[C@@H]1NC(=O)[C@H]([C@H](O)[C@H](C)CCCC(=O)O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

A flame-dried 10-mL microwave vial was flushed with dry argon, and then was charged with cyclosporin A (100 mg, 0.083 mmol), 1,2-dichloroethane (1.24 mL), and a magnetic stir bar. tert-Butyl acrylate (0.24 mL, 1.66 mmol) and Grubbs-Hoveyda Catalyst 2nd Gen (3.5 mg, 0.042 mmol) were added sequentially. The vial was flushed with argon again and sealed with a rubber cap. The reaction mixture was heated at 70° C. for 1 h in a CEM Discover SP microwave reactor with constant stirring. After cooling to 23° C., LC-MS analysis showed ˜50% conversion to the desired product mass. The vial was returned to the microwave reactor and heated for an additional 3 h at 70° C. The reaction mixture was cooled to 23° C. and directly loaded onto a silica gel cartridge (˜4 g). Purification by column chromatography (0-20% methanol-dichloromethane, 4-g RediSep Rf Column, Teledyne ISCO, Lincoln, Nebr.) afforded the product as a brown solid. The purity of this material was ˜80% by ¹H NMR analysis.

Trifluoroacetic acid (0.5 mL) was added to a solution of the product from the cross-metathesis reaction in dichloromethane (0.5 mL). In 1 h, LC-MS analysis showed full consumption of the tert-butyl ester starting material (m/z=1288). The reaction mixture was concentrated under vacuum. The residue was diluted with 50% acetonitrile-water to a volume of 4.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (31 mg, 31% over 2 steps).

A 100-mL round bottom flask was charged with 06-058 (412 mg, 0.33 mmol), 1:1 Ethyl acetate (6.6 mL):methanol (6.6 mL) and a magnetic stir bar. Argon was bubbled through the solution for 5 min, then Palladium on carbon (10 wt %, 71 mg, 0.033 mmol) was added. The vessel was fitted with a rubber septum and a hydrogen balloon was attached via a 19-gauge needle. An additional needle was attached to allow a gentle flow of hydrogen to bubble through the solution at a continuous rate. At 3 h, LC-MS could no longer detect any starting material. The hydrogen balloon was switched to one filled with argon, and bubbling was continued for 5 min. The reaction mixture was then filtered through a tightly packed plug of Celite (˜2 g). Concentration of the filtrate afforded a colorless glass. The material was purified by reverse-phase HPLC in multiple batches with the following procedure. The residue was divided into batches and dissolved in 50% methanol-water (100 mg in 5 mL, 150 mg in 8 mL, then 150 mg in 8 mL), and the solutions were filtered through a 0.45 μM PTFE syringe filter. The filtrate was purified by reverse-phase HPLC (Waters XBridge C18 column 5 μm particle size 30×250 mm, 50-95% acetonitrile-water+0.1% formic acid, 40 min, 20 mL/min) in batches, and the product-containing fractions were pooled to afford the product as a white solid (343 mg, 83%). ¹H NMR (400 MHz, Chloroform-d) δ 7.99 (d, J=9.2 Hz, 1H), 7.63 (d, J=7.5 Hz, 1H), 5.68 (dd, J=11.0, 4.1 Hz, 1H), 5.39 (d, J=7.3 Hz, 1H), 5.31-5.23 (m, 1H), 5.17-5.05 (m, 3H), 5.00 (q, J=7.5 Hz, 1H), 4.88-4.81 (m, 1H), 4.72 (d, J=14.3 Hz, 1H), 4.63 (t, J=8.8 Hz, 1H), 4.51 (dt, J=14.6, 7.5 Hz, 1H), 3.87 (t, J=6.3 Hz, 1H), 3.42 (s, 3H), 3.40-3.35 (m, 4H), 3.30 (d, J=11.8 Hz, 1H), 3.23 (s, 3H), 3.19 (s, 3H), 3.18 (d, J=17.2 Hz, 1H), 3.09 (s, 3H), 2.87 (d, J=17.6 Hz, 1H), 2.78-2.68 (m, 1H), 2.69 (s, 3H), 2.67 (s, 3H), 2.48-2.30 (m, 2H), 2.23 (t, J=6.9 Hz, 2H), 2.22-1.87 (m, 5H), 1.80-1.37 (m, 13H), 1.34 (d, J=7.3 Hz, 3H), 1.26 (d, J=6.9 Hz, 3H), 1.24-1.12 (m, 3H), 1.07-0.79 (m, 39H, 13 methyl doublets). HRMS (ESI): Calcd for (C₆₂H₁₁₁N₁₁O₁₄−H)⁺: 1232.8234, Found: 1232.8215.

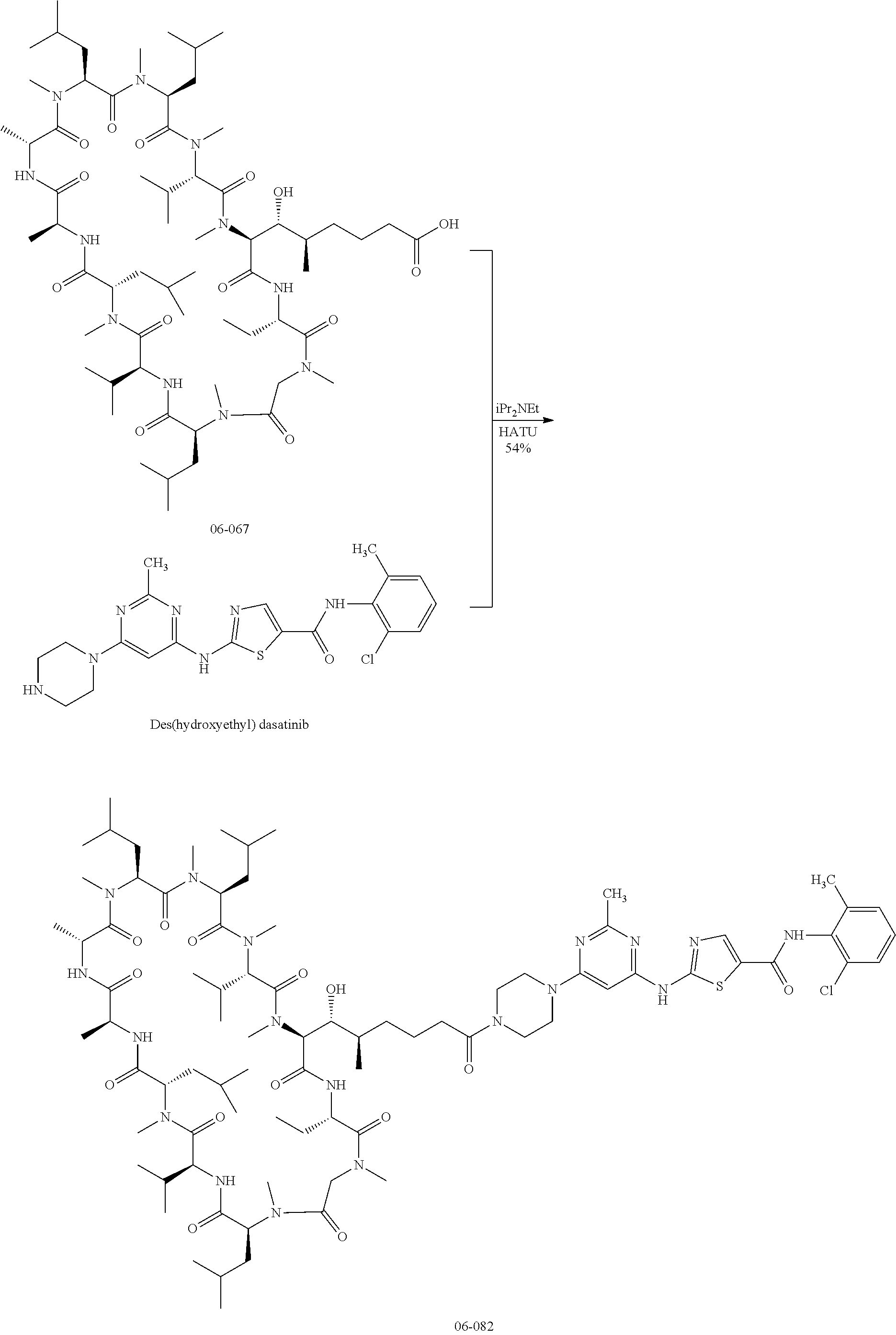

A 1-dram vial was charged with Cyclosporin C4 Acid (20 mg, 0.016 mmol), des(hydroxyethyl)dasatinib (7.9 mg, 0.018 mmol), DMF (0.20 mL), N,N-Diisopropylethylamine (8.5 μL, 0.049 mmol) and a magnetic stir bar. The solution was cooled to 0° C., then HATU (7.4 mg, 0.019 mmol) was added as a solid. The resulting mixture was stirred at 0° C. In 30 min, LC-MS analysis indicated that the starting material had been fully consumed. The reaction mixture was diluted with 50% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (14.5 mg, 54%). ¹H NMR spectrum of this compound contains at least three conformational isomers and cannot be resolved. HRMS (ESI): Calcd for (C₈₂H₁₃₁ClN₁₈O₁₄S+2H)²⁺: 830.4789, Found: 830.4791.

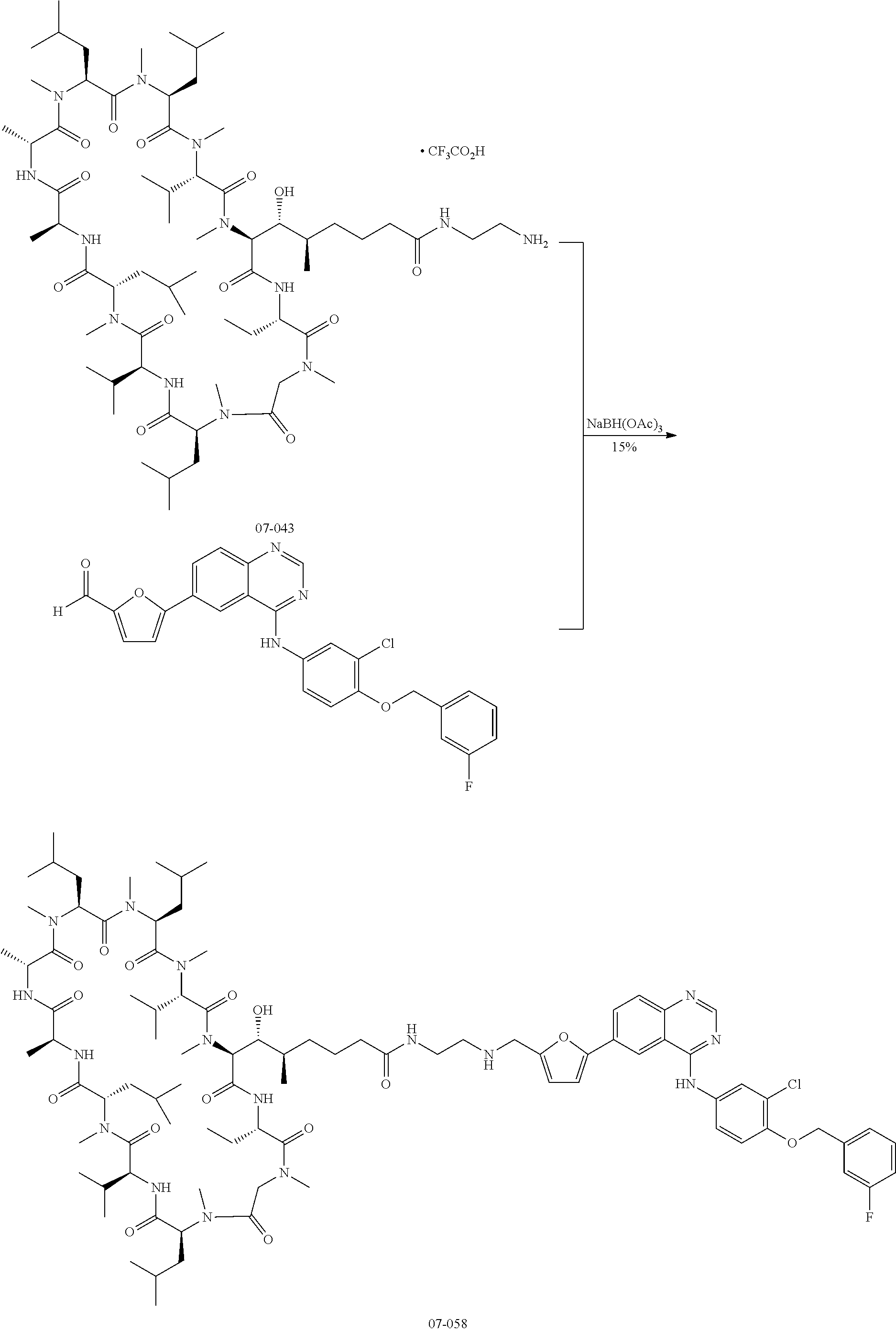

An oven-dried 1-dram vial was charged with 07-043 (15 mg, 0.011 mmol), Lapatinib aldehyde (5.0 mg, 0.011 mmol), dichloromethane (0.11 mL) and a magnetic stir bar. Sodium triacetoxyborohydride (4.5 mg, 0.021 mmol) was added as a solid. In about 5 min, all the solids had gone into solution. The reaction mixture was kept stirred for a total of 2 h, at which point LC-MS still showed presence of both starting materials. Additional sodium triacetoxyborohydride (4.5 mg, 0.021 mmol) was added. In a total of 6 h, LC-MS showed full consumption of the amine starting material. The reaction solution was concentrated to dryness until vacuum. The residue was diluted with 50% acetonitrile-water to a volume of 3.5 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a yellow solid (2.7 mg, 15%). ¹H NMR spectrum of this compound contains at least three conformational isomers and cannot be resolved. HRMS (ESI): Calcd for (C₉₀H₁₃₄ClFN₁₆O₁₅+2H)²⁺: 867.5021, Found: 867.5022.

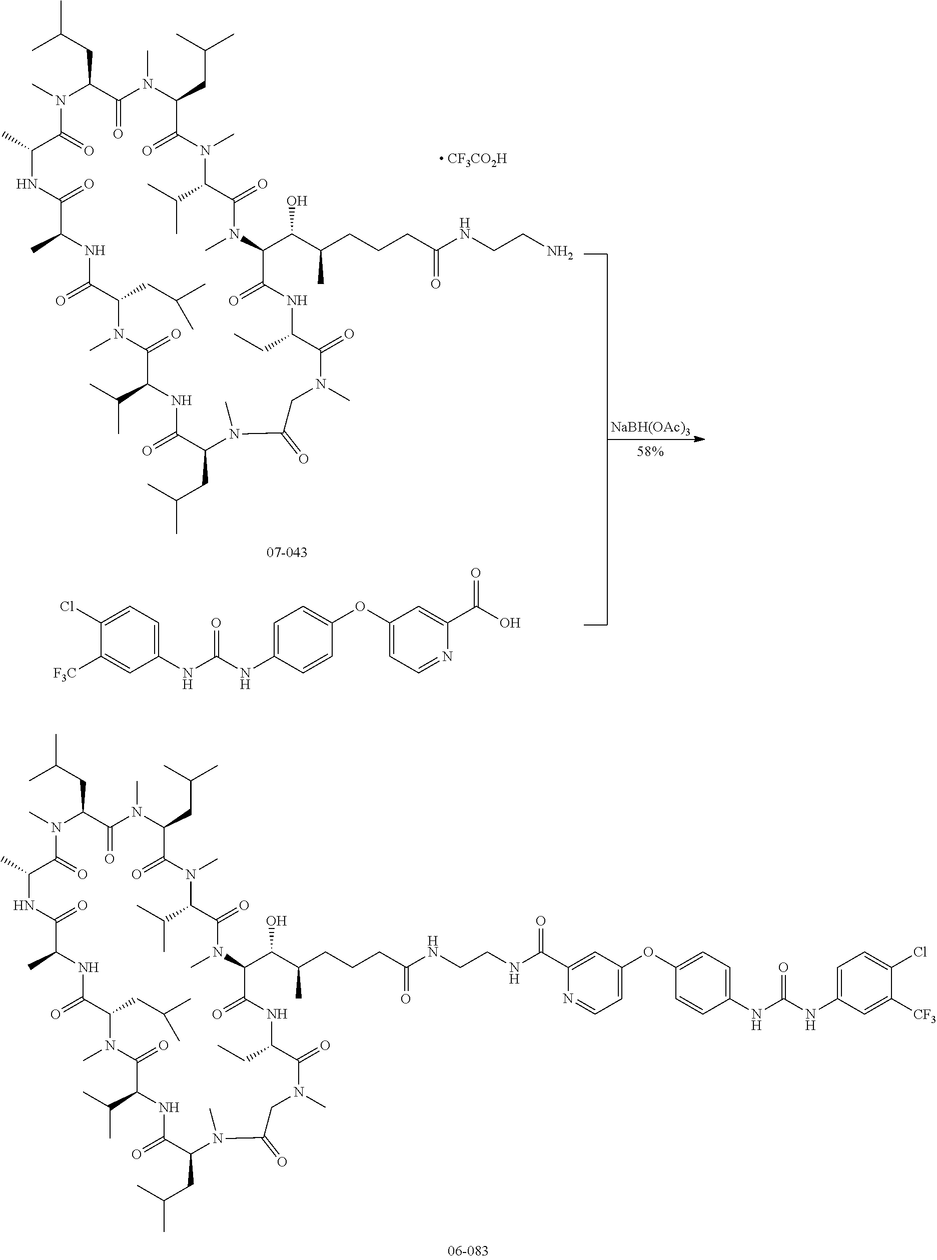

A 1-dram vial was charged with 07-043 (20 mg, 0.016 mmol), sorafenib acid (11 mg, 0.018 mmol), DMF (0.20 mL), N,N-Diisopropylethylamine (8.5 μL, 0.048 mmol) and a magnetic stir bar. The solution was cooled to 0° C., then HATU (7.4 mg, 0.019 mmol) was added as a solid. The resulting mixture was stirred at 0° C. and the reaction progress was monitored by LC-MS. In 30 min, LC-MS analysis indicated that the starting material had been fully consumed. The reaction mixture was diluted with 50% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (16.2 mg, 58%). ¹H NMR spectrum of this compound contains at least three conformational isomers and cannot be resolved. HRMS (ESI): Calcd for (C₈₄H₁₂₈ClF₃N₁₆O₁₆+2H)⁺: 855.4746, Found: 855.4745.

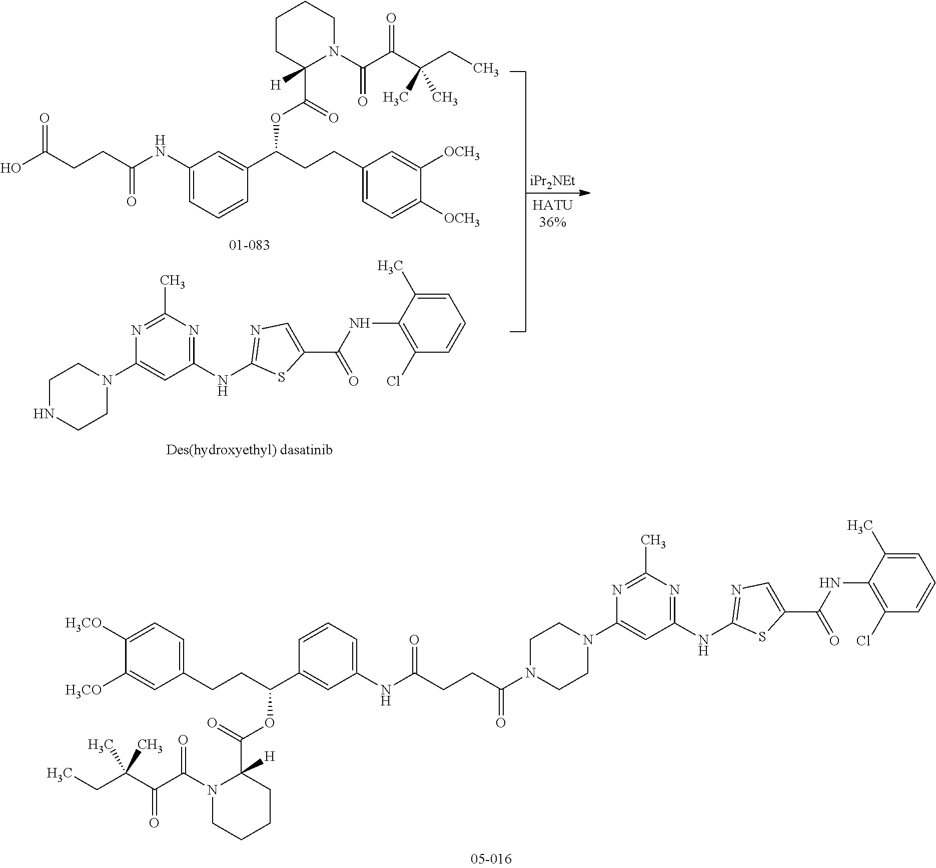

An oven-dried 1-dram vial was charged with des(hydroxyethyl)dasatinib (10 mg, 0.023 mmol), 01-083 (14 mg, 0.023 mmol), DMF (0.11 mL), N,N-diisopropylethylamine (12 μL, 0.068 mmol) and a magnetic stir bar. The solution was cooled to 0° C., then HATU (12.8 mg, 0.034 mmol) was added as a solid. The resulting mixture was stirred at 0° C. and the reaction progress was monitored by LC-MS. In 30 min, LC-MS analysis indicated that the starting material had been fully consumed. The residue was diluted with 50% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (8.5 mg, 36%). 6:1 mixture of rotamers. ¹H NMR (400 MHz, Chloroform-d) δ 9.80 (br s, 1H), 8.10 (br s, 1H), 7.86 (br s, 1H), 7.66 (d, J=8.1 Hz, 1H), 7.40-7.27 (m, 2H), 7.24-7.13 (m, 3H), 6.96 (d, J=7.8 Hz, 1H), 6.79-6.71 (m, 1H), 6.70-6.58 (m, 2H), 5.98 (s, 1H), 5.69 (t, J=6.8 Hz, 1H), 5.22 (d, J=5.6 Hz, 1H), 3.95-3.84 (m, 1H), 3.83 (s, 3H), 3.82 (s, 3H), 3.81-3.71 (m, 1H), 3.66 (d, 4H), 3.45-3.39 (m, 4H), 3.27 (d, J=13.6 Hz, 1H), 3.05 (t, J=13.1 Hz, 1H), 2.95-2.76 (m, 3H), 2.52 (q, 5H), 2.34 (s, 3H), 2.27-2.11 (m, 2H), 2.08-1.93 (m, 1H), 1.72-1.46 (m, 5H), 1.46-1.30 (m, 2H), 1.17 (s, 3H), 1.16 (s, 3H), 0.85 (t, J=7.4 Hz, 3H). HRMS (ESI): Calcd for (C₅₄H₆₄ClN₉O₉S+H)⁺: 1050.4314, Found: 1050.4298.

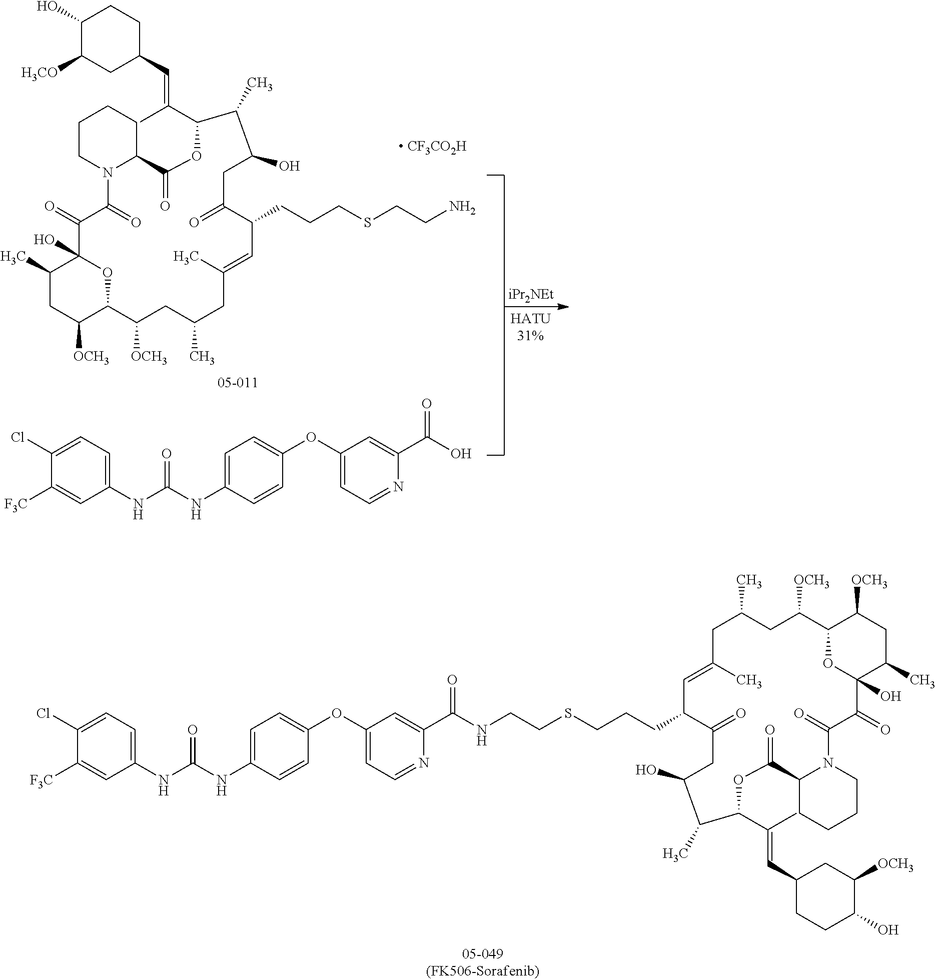 [SMILES: "iPr2NEt" ; CO[C@H]1C[C@@H](C)C/C(C)=C/[C@@H](CCCSCCN)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; "· CF3CO2H" ; O=C(Nc1ccc(Oc2ccnc(C(=O)O)c2)cc1)Nc1ccc(Cl)c(C(F)(F)F)c1 ; CO[C@H]1C[C@@H](C)C/C(C)=C/[C@@H](CCCSCCNC(=O)c2cc(Oc3ccc(NC(=O)Nc4ccc(Cl)c(C(F)(F)F)c4)cc3)ccn2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

A 1-dram vial was charged with 05-011 (22 mg, 0.022 mmol), 4-[4-[[4-chloro-3-(trifluoromethyl)phenyl]carbamoylamino]phenoxy]pyridine-2-carboxylic acid (10 mg, 0.022 mmol), DMF (0.11 mL), and a magnetic stir bar. The resulting mixture was stirred until all reactants had dissolved. N,N-diisopropylamine (12 μL, 0.066 mmol) and HATU (10 mg, 0.026 mmol) were added sequentially at 23° C., and the resulting solution was stirred at 23° C. for 15 min. LC-MS analysis at this point showed full consumption of the starting material and formation of the desired product. The reaction mixture was diluted with 50% acetonitrile-water to a volume of 4.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (9.1 mg, 31%). 3:2 mixture of rotamers. ¹H NMR (400 MHz, Chloroform-d) δ 8.51-8.37 (m, 2H), 7.93 (s, 1H), 7.82-7.68 (m, 2H), 7.60-7.49 (m, 2H), 7.49-7.35 (m, 1H), 7.18-7.12 (m, 1H), 7.12-7.00 (m, 2H), 5.36 (s, 1H), 5.12-4.99 (m, 2H), 4.62 (s, 1H), 4.43 (d, J=13.4 Hz, 1H), 4.34 (s, 1H), 4.04-3.97 (m, 1H), 3.73-3.63 (m, 2H), 3.63-3.48 (m, 2H), 3.42 (s, 3H), 3.41 (s, 3H), 3.40-3.36 (m, 3H), 3.33 (s, 3H), 3.09-2.99 (m, 1H), 2.94 (t, J=12.1 Hz, 1H), 2.85-2.66 (m, 4H), 2.54 (t, J=7.5 Hz, 2H), 2.40-2.24 (m, 2H), 2.23-1.94 (m, 5H), 1.94-1.60 (m, 6H), 1.57 (d, J=8.4 Hz, 6H), 1.55-1.26 (m, 8H), 1.12-0.99 (m, 2H), 0.96 (d, J=6.3 Hz, 3H), 0.88 (d, J=7.2 Hz, 3H). HRMS (ESI): Calcd for (C₆₆H₈₇ClF₃N₅O₁₅S+H)⁺: 1314.5638, Found: 1314.5674.

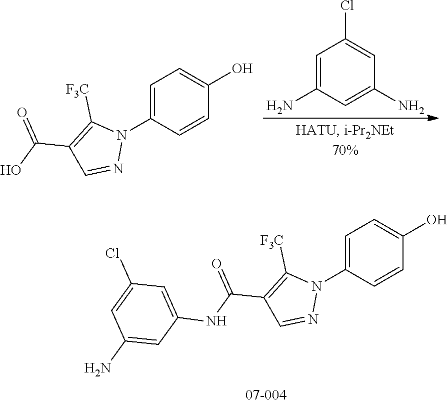 [SMILES: O=C(O)c1cnn(-c2ccc(O)cc2)c1C(F)(F)F ; Nc1cc(N)cc(Cl)c1 ; Nc1cc(Cl)cc(NC(=O)c2cnn(-c3ccc(O)cc3)c2C(F)(F)F)c1]

An oven-dried 20-mL vial was charged with 1-(4-hydroxyphenyl)-5-(trifluoromethyl)pyrazole-4-carboxylic acid (50 mg, 0.18 mmol), 5-Chloro-1,3-benzenediamine (131 mg, 0.92 mmol) and a magnetic stir bar. DMF (0.37 mL) was added and the mixture was stirred until all reactants had dissolved. N,N-Diisopropylethylamine (96 μL, 0.55 mmol) and HATU (91 mg, 0.24 mmol) were added sequentially. Stirring was continued and the reaction progress was monitored by LC-MS. In 8 h, LC-MS analysis showed full consumption of the starting material and formation of one major product and one minor product. The minor product had an m/z that matched a dimer (bis-acylation). The reaction mixture was diluted with 50% acetonitrile-water to a volume of 10.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (51 mg, 70%). ¹H NMR (400 MHz, Methanol-d₄) δ 8.05 (s, 1H), 7.30 (d, J=8.8 Hz, 2H), 7.03-6.98 (m, 2H), 6.98-6.89 (m, 2H), 6.52 (t, J=1.9 Hz, 1H). HRMS (ESI): Calcd for (C₁₇H₁₂ClF₃N₄O₂+H)⁺: 397.0679, Found: 397.0689.

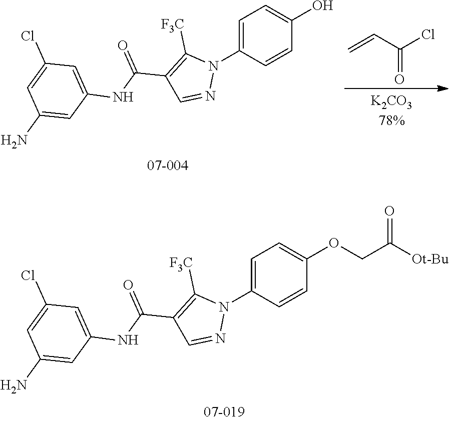

An oven-dried 1-dram vial was charged with N-(3-amino-5-chloro-phenyl)-1-(4-hydroxyphenyl)-5-(trifluoromethyl)pyrazole-4-carboxamide (30 mg, 0.076 mmol), Potassium carbonate (21 mg, 0.15 mmol), DMF (0.13 mL), and a magnetic stir bar. Tert-butyl bromoacetate (11 μL, 0.076 mmol) was added via pipette, and the resulting mixture was stirred at 23° C. In 16 h, LC-MS indicated that the starting material had been fully consumed and two products had formed. One had the desired m/z and the other seemed to be a bis-alkylation product. The reaction mixture was partitioned between saturated aqueous sodium bicarbonate solution (5 mL) and ethyl acetate (5 mL). The layers were separated, and the aqueous layer was extracted with ethyl acetate (2×5 mL). The combined organic layers were dried over sodium sulfate. The dried solution was filtered, and the filtrate was concentrated. The residue was purified by column chromatography (20-80% ethyl acetate-hexanes, 4-g RediSep(R) Rf column, Teledyne ISCO, Lincoln, Nebr.) to afford the product as a yellow powder (30 mg, 78%). ¹H NMR (400 MHz, Chloroform-d) δ 7.96 (s, 1H), 7.51 (s, 1H), 7.40-7.32 (m, 2H), 7.10 (d, J=2.3 Hz, 1H), 7.04-6.94 (m, 2H), 6.79 (t, J=1.8 Hz, 1H), 6.47 (t, J=1.9 Hz, 1H), 4.58 (s, 2H), 3.83 (s, 2H), 1.49 (s, 9H). HRMS (ESI): Calcd for (C₂₃H₂₂ClF₃N₄O₄+H)⁺: 511.1360, Found: 511.1376.

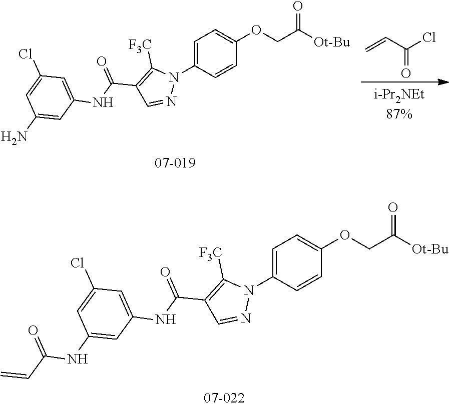

A solution of tert-butyl 2-[4-[4-[(3-amino-5-chloro-phenyl)carbamoyl]-5-(trifluoromethyl)pyrazol-1-yl]phenoxy]acetate (30 mg, 0.060 mmol) in dichloromethane (0.39 mL) was cooled to 0° C. Triethylamine (16.37 μL, 0.12 mmol) and Acryloyl chloride (5.7 μL, 0.071 mmol) were added sequentially via syringe. The resulting solution was stirred at 0° C. for 30 min, at which point TLC analysis (50% ethyl acetate-hexanes) indicated full conversion of the starting material to a slightly less polar spot. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (20-80% ethyl acetate-hexanes, 4-g RediSep(R) Rf column, Teledyne ISCO, Lincoln, Nebr.) to afford the product as a yellow powder (29 mg, 87%). ¹H NMR (400 MHz, Chloroform-d) δ 8.81 (s, 1H), 8.33 (s, 1H), 7.94 (s, 1H), 7.82-7.75 (m, 1H), 7.38 (dt, J=11.3, 1.8 Hz, 2H), 7.27 (d, J=7.1 Hz, 1H), 7.00-6.88 (m, 2H), 6.32 (dd, J=16.9, 1.4 Hz, 1H), 6.18 (dd, J=16.9, 10.2 Hz, 1H), 5.68 (dd, J=10.1, 1.4 Hz, 1H), 4.55 (s, 2H), 1.48 (s, 9H). HRMS (ESI): Calcd for (C₂₆H₂₄ClF₃N₄O₅+H)⁺: 565.1466, Found: 565.1466.

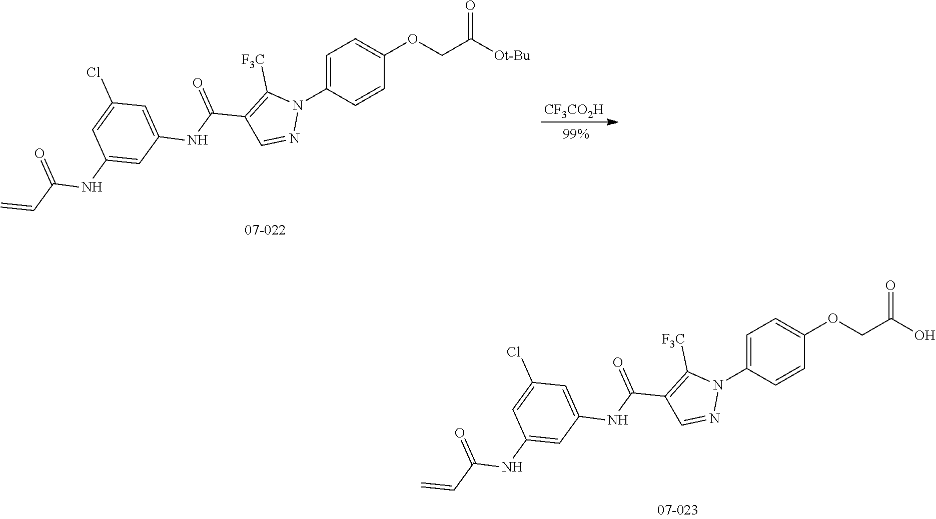 [SMILES: C=CC(=O)Nc1cc(Cl)cc(NC(=O)c2cnn(-c3ccc(OCC(=O)OC(C)(C)C)cc3)c2C(F)(F)F)c1 ; "CF3CO2H" ; C=CC(=O)Nc1cc(Cl)cc(NC(=O)c2cnn(-c3ccc(OCC(=O)O)cc3)c2C(F)(F)F)c1]

tert-butyl 2-[4-[4-[[3-chloro-5-(prop-2-enoylamino)phenyl]carbamoyl]-5-(trifluoromethyl)pyrazol-1-yl]phenoxy]acetate (29 mg, 0.050 mmol) was dissolved in 1:1 dichloromethane (0.2000 mL):trifluroacetic Acid (0.2000 mL) and the resulting solution was allowed to stand at 23° C. for 1 h. The solution was then concentrated to afford the product as a white solid (26 mg, 99%). HRMS (ESI): Calcd for (C₂₂H₁₆ClF₃N₄O₅+H)⁺: 509.0840, Found: 509.0847.

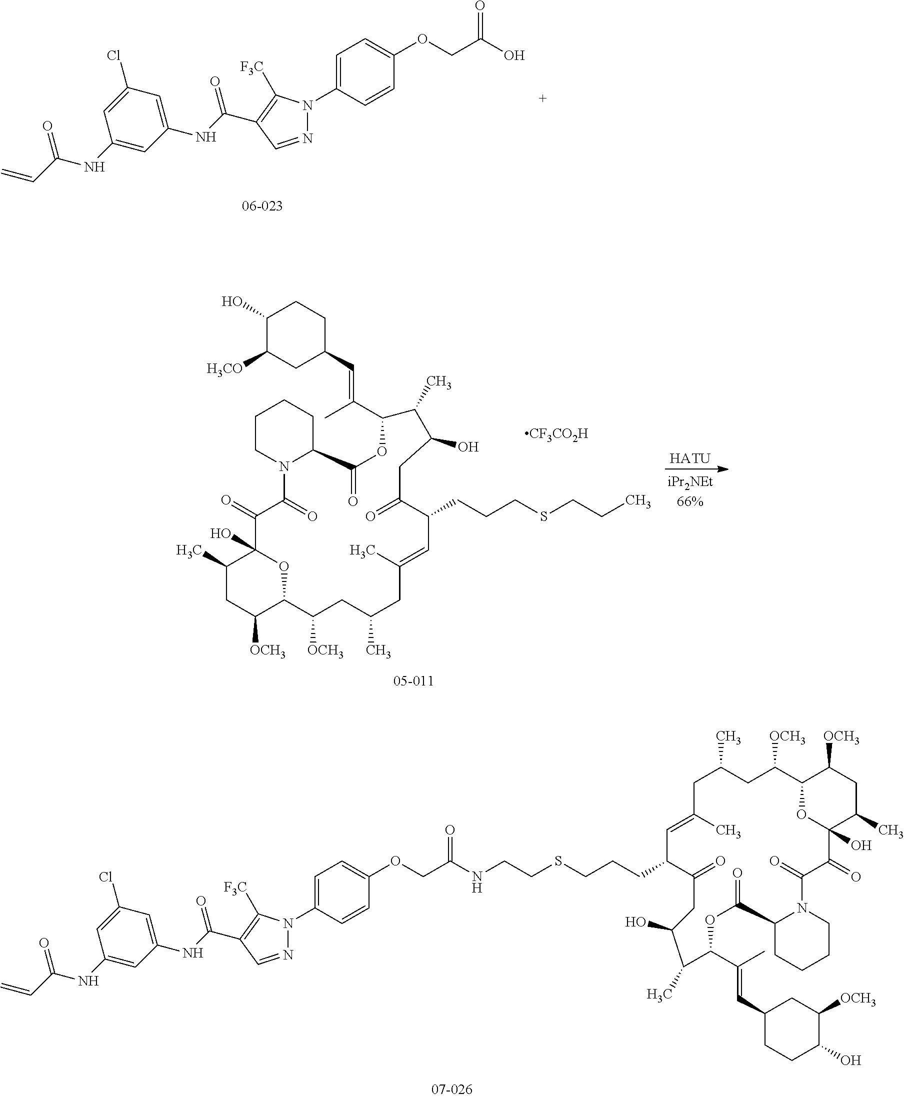 [SMILES: C=CC(=O)Nc1cc(Cl)cc(NC(=O)c2cnn(-c3ccc(OCC(=O)O)cc3)c2C(F)(F)F)c1 ; CCCSCCC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC ; "·CF3CO2H" ; "HATU" ; C=CC(=O)Nc1cc(Cl)cc(NC(=O)c2cnn(-c3ccc(OCC(=O)NCCSCCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)cc3)c2C(F)(F)F)c1]

An oven-dried 1-dram vial was charged with 06-023 (7.7 mg, 0.015 mmol), 05-011 (15 mg, 0.015 mmol), DMF (0.11 mL), and a magnetic stir bar. N,N-Diisopropylethylamine (7.9 μL, 0.045 mmol) and HATU (6.9 mg, 0.018 mmol) were added sequentially, and the resulting solution was stirred at 23° C. Within 30 min, LC-MS analysis showed that the starting material (FK-amine) was fully consumed. The residue was diluted with 1:1:1 methanol-acetonitrile-water to a volume of 5.0 mL, and the solution was filtered through a 0.45 μM PTFE syringe filter. The filtrate was purified by reverse-phase HPLC (Waters XBridge C₁₈ column 5 μm particle size 30×250 mm, 5-95% acetonitrile-water+0.1% formic acid, 40 min, 20 mL/min) to afford the product as a white solid (13.7 mg, 66%). ¹H NMR (400 MHz, Chloroform-d) δ 8.37 (s, 1H), 8.07-7.99 (m, 1H), 7.65 (s, 1H), 7.59 (s, 1H), 7.45 (d, J=8.8 Hz, 2H), 7.09 (d, J=8.9 Hz, 2H), 6.47 (dd, J=16.8, 1.3 Hz, 1H), 6.28 (dd, J=16.8, 10.3 Hz, 1H), 5.81 (d, J=9.9 Hz, 1H), 5.32 (s, 1H), 5.11-4.97 (m, 2H), 4.66-4.62 (m, 3H), 4.43 (d, J=13.3 Hz, 1H), 4.22 (s, 1H), 3.95-3.85 (m, 2H), 3.68 (d, J=9.4 Hz, 1H), 3.64-3.51 (m, 4H), 3.42 (s, 3H), 3.41 (s, 3H), 3.41-3.37 (m, 3H), 3.32 (s, 3H), 3.08-2.98 (m, 2H), 2.70 (app t, J=6.4 Hz, 4H), 2.43 (d, J=7.0 Hz, 1H), 2.38-2.24 (m, 2H), 2.24-2.13 (m, 3H), 2.12-1.86 (m, 5H), 1.85-1.68 (m, 6H), 1.66-1.54 (m, 6H), 1.53-1.25 (m, 8H), 1.10-1.03 (m, 2H), 1.00 (d, J=6.3 Hz, 3H), 0.96 (d, J=6.2 Hz, 3H), 0.88 (d, J=7.1 Hz, 3H). HRMS (ESI): Calcd for (C₆₈H₉₀ClF₃N₆O₁₆S−H)⁻: 1369.5697, Found: 1369.5664.

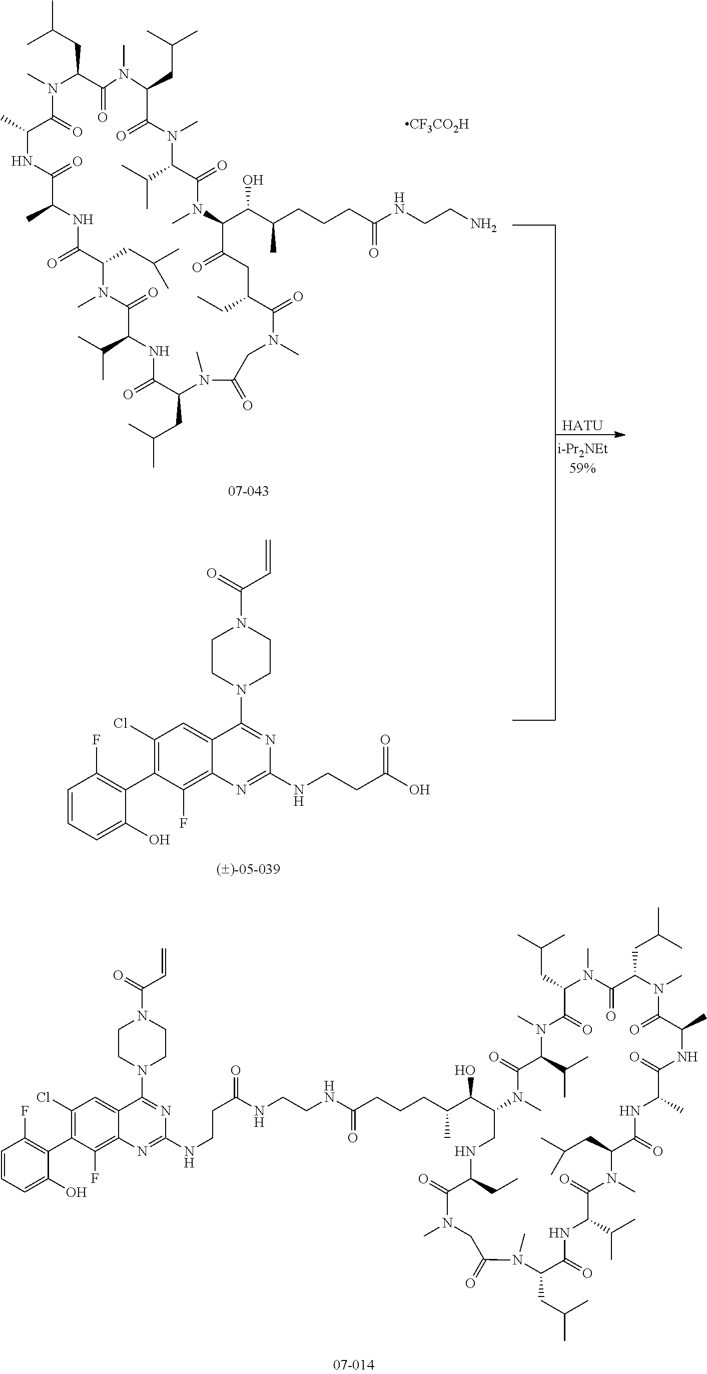

An oven dried one-dram vial was charged with 07-043 (10 mg, 0.076 mmol), 05-039 (4.3 mg, 0.083 mmol), DMF (0.10 mL), and a magnetic stir bar. N,N-Diisopropylethylamine (4.0 μL, 0.023 mmol) and HATU (4.3 mg, 0.011 mmol) were added sequentially to the reaction mixture at 23° C. In 40 min, LC-MS analysis showed 100% conversion. The reaction mixture was diluted with 50% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white powder (7.9 mg, 58%). HRMS (ESI): Calcd for (C₈₈H₁₃₇ClF₂N₁₈O₁₆+2H)⁺: 888.5136, Found: 888.5137.

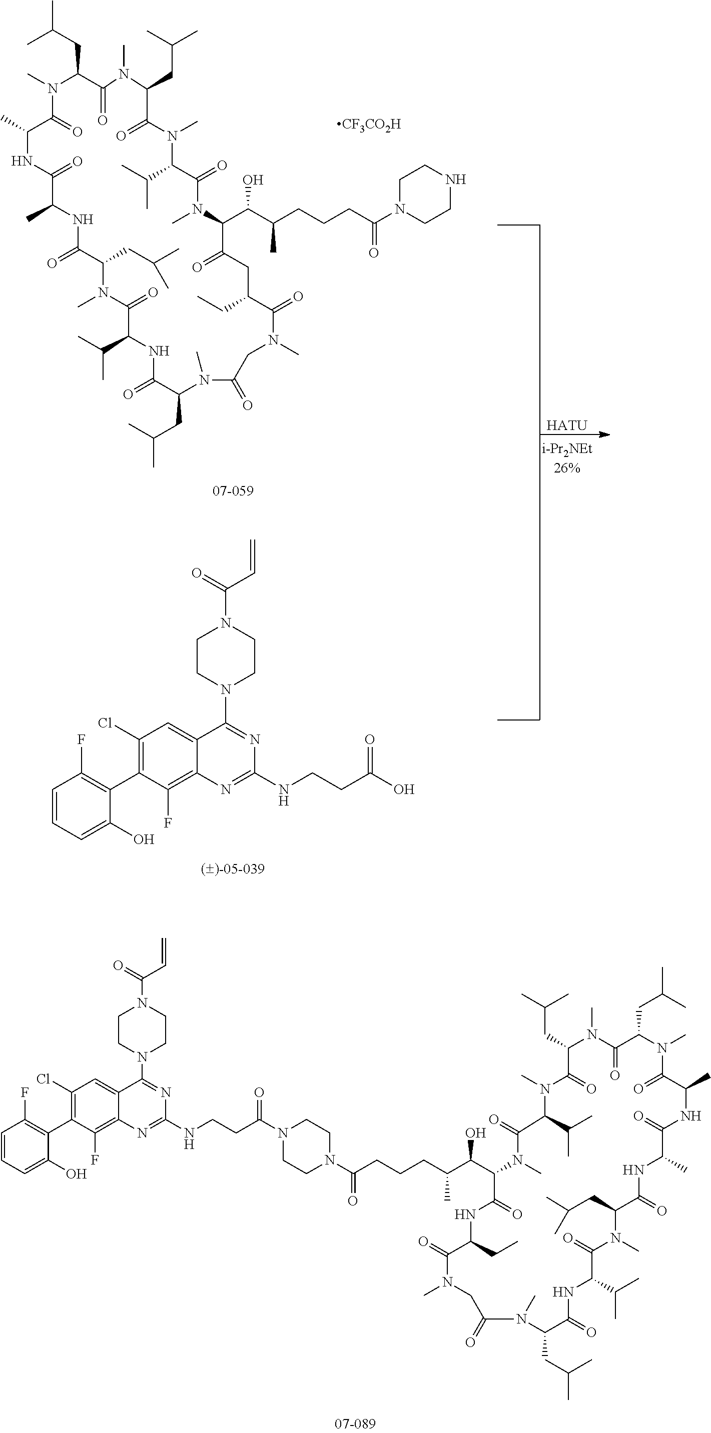

An oven dried one-dram vial was charged with 07-059 (10 mg, 0.071 mmol), 05-039 (4.0 mg, 0.078 mmol), DMF (0.10 mL), and a magnetic stir bar. N,N-Diisopropylethylamine (3.7 μL, 0.023 mmol) and HATU (3.0 mg, 0.011 mmol) were added sequentially to the reaction mixture at 23° C. In 1 h, LC-MS analysis showed full consumption of the starting material. The reaction mixture was diluted with 50% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white powder (3.3 mg, 26%). HRMS (ESI): Calcd for (C₉₀H₁₃₉ClF₂N₁₈O₁₆+2H)⁺: 901.5214, Found: 901.5244.

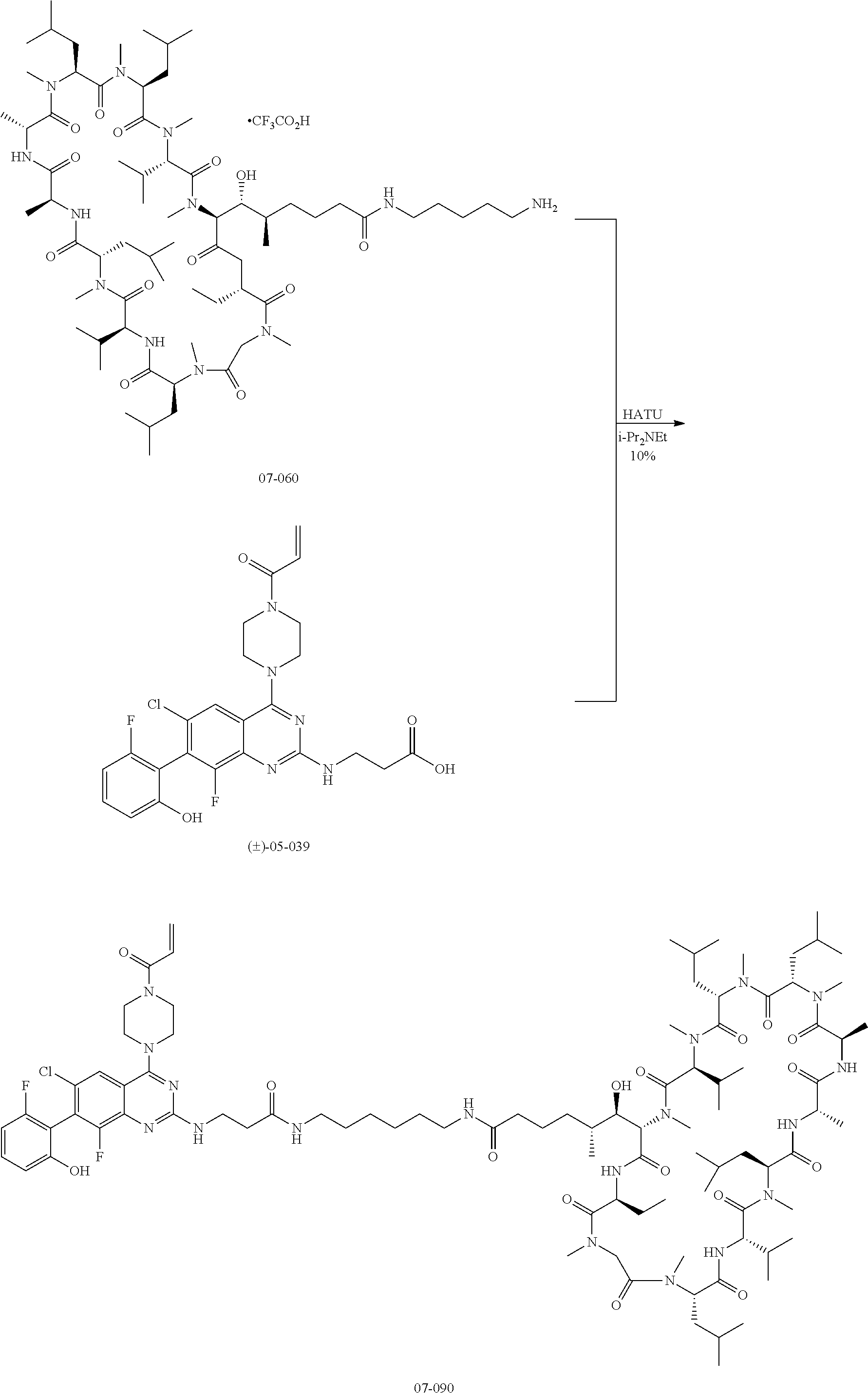

An oven dried one-dram vial was charged with 07-060 (10 mg, 0.071 mmol), 05-039 (4.0 mg, 0.078 mmol), DMF (0.10 mL), and a magnetic stir bar. N,N-Diisopropylethylamine (3.7 μL, 0.023 mmol) and HATU (3.0 mg, 0.011 mmol) were added sequentially to the reaction mixture at 23° C. In 1 h, LC-MS analysis showed full consumption of the starting material. The reaction mixture was diluted with 50% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white powder (1.3 mg, 10%). HRMS (ESI): Calcd for (C₉₂H₁₄₅ClF₂N₁₈O₁₆+2H)⁺: 916.5450, Found: 916.5455.

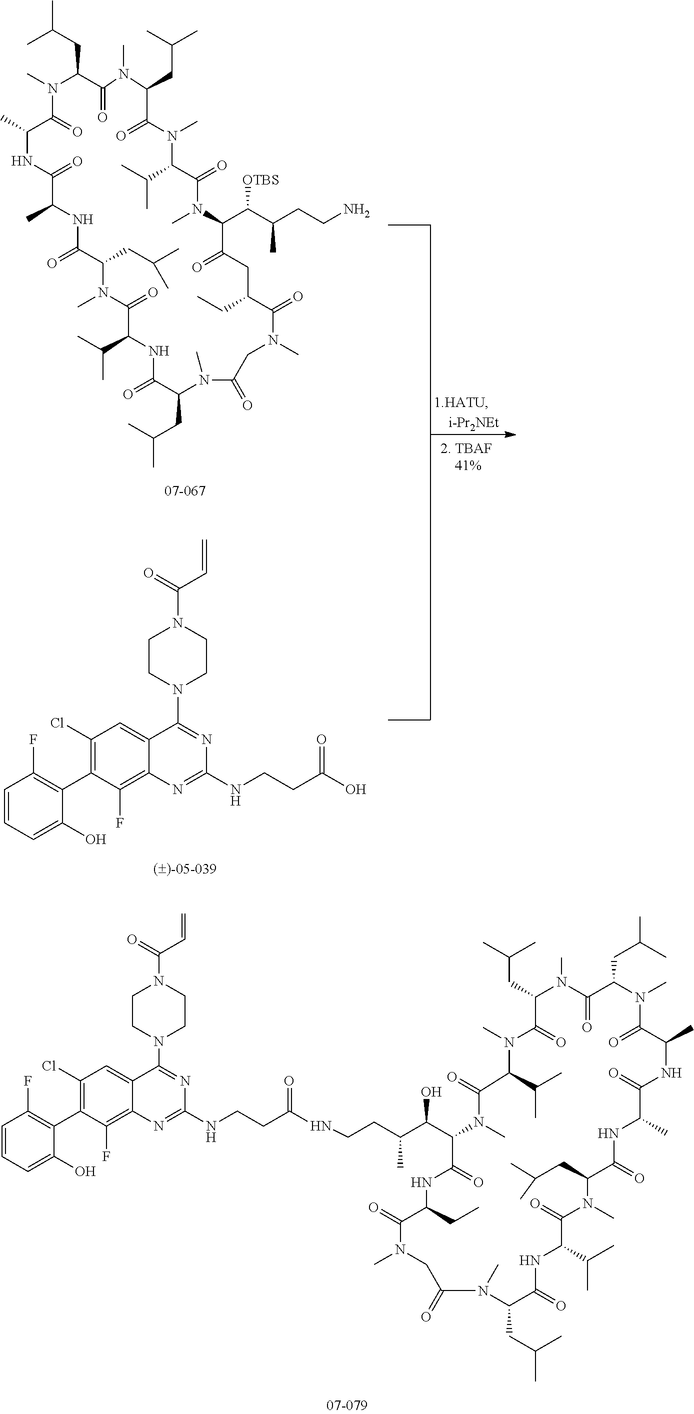

A 1-dram vial was charged with 07-067 (7.0 mg, 0.0054 mmol), 05-039 (3.3 mg, 0.0064 mmol) and a magnetic stir bar. DMF (0.10 mL) and N,N-Diisopropylethylamine (4.7 μL, 0.027 mmol) were added sequentially via pipette. HATU (2.5 mg, 0.0064 mmol) was added as a freshly prepared 10% w/v DMF solution via pipette. The resulting solution was stirred at 23° C. and the reaction progress was monitored by LC-MS. In 12 h, LC-MS analysis showed full consumption of the amine starting material and formation of a new species. The reaction mixture was diluted with 0.1 mL THF, and a 1.0 M solution of tetra-n-butylammonium fluoride in THE (27 μL, 0.027 mmol) was added dropwise via syringe. In 2 h, LC-MS showed full deprotection of the TBS group. The residue was diluted with 50% acetonitrile-water to a volume of 3.9 mL, and the solution was filtered through a 0.45 μM PTFE syringe filter. The filtrate was purified by reverse-phase HPLC (Waters XBridge C18 column 5 μm particle size 30×250 mm, 50-95% acetonitrile-water+0.1% formic acid, 40 min, 20 mL/min) to afford the product as a white solid (3.7 mg, 41%). HRMS (ESI): Calcd for (C₈₄H₁₃₀ClF₂N₁₇O₁₅−H)⁻: 1688.9511, Found: 1688.9529.

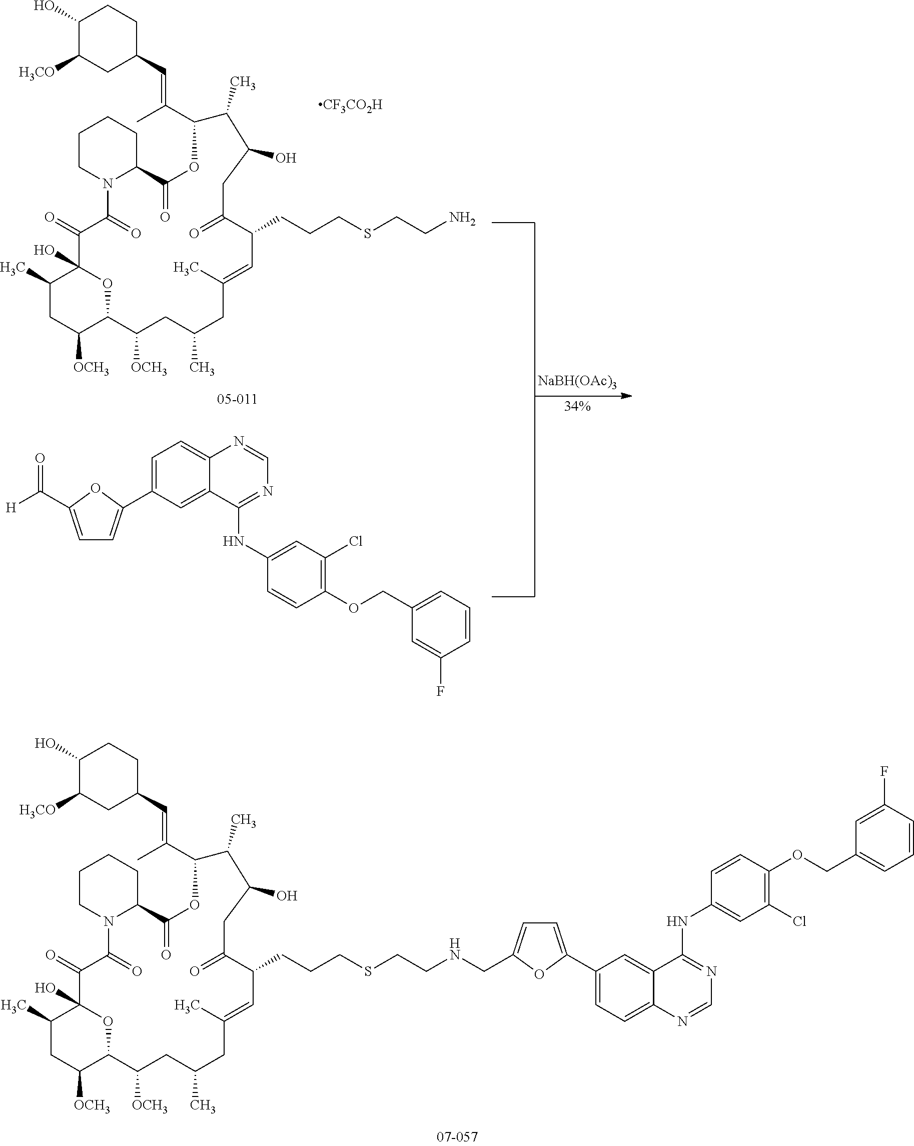 [SMILES: CO[C@H]1C[C@@H](C)C/C(C)=C/[C@@H](CCCSCCN)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; "·CF3CO2H" ; O=Cc1ccc(-c2ccc3ncnc(Nc4ccc(OCc5cccc(F)c5)c(Cl)c4)c3c2)o1 ; CO[C@H]1C[C@@H](C)C/C(C)=C/[C@@H](CCCSCCNCc2ccc(-c3ccc4ncnc(Nc5ccc(OCc6cccc(F)c6)c(Cl)c5)c4c3)o2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; "NaBH(OAc)3"]

An oven-dried one-dram vial was charged with 05-011 (10.5 mg, 0.0106 mmol), Lapatinib aldehyde (5.0 mg, 0.0106 mmol) and a magnetic stir bar. DCM (0.11 mL) was added via syringe. The resulting mixture was stirred at 23° C. for 10 min. The aldehyde reactant did not fully dissolve. Sodium triacetoxyborohydride (4.5 mg, 0.0211 mmol) was added as a solid. In about 5 min, all the solids had gone into solution. The reaction mixture was kept stirred for a total of 2 h, at which point no starting material amine could be detected. The reaction solution was concentrated to dryness under vacuum. The residue was diluted with 50% acetonitrile-water to a volume of 3.5 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a yellow solid (4.8 mg, 34%). HRMS (ESI): Calcd for (C₇₂H₉₃ClFN₅O₁₄S+2H)²+: 669.8134, Found: 669.7951.

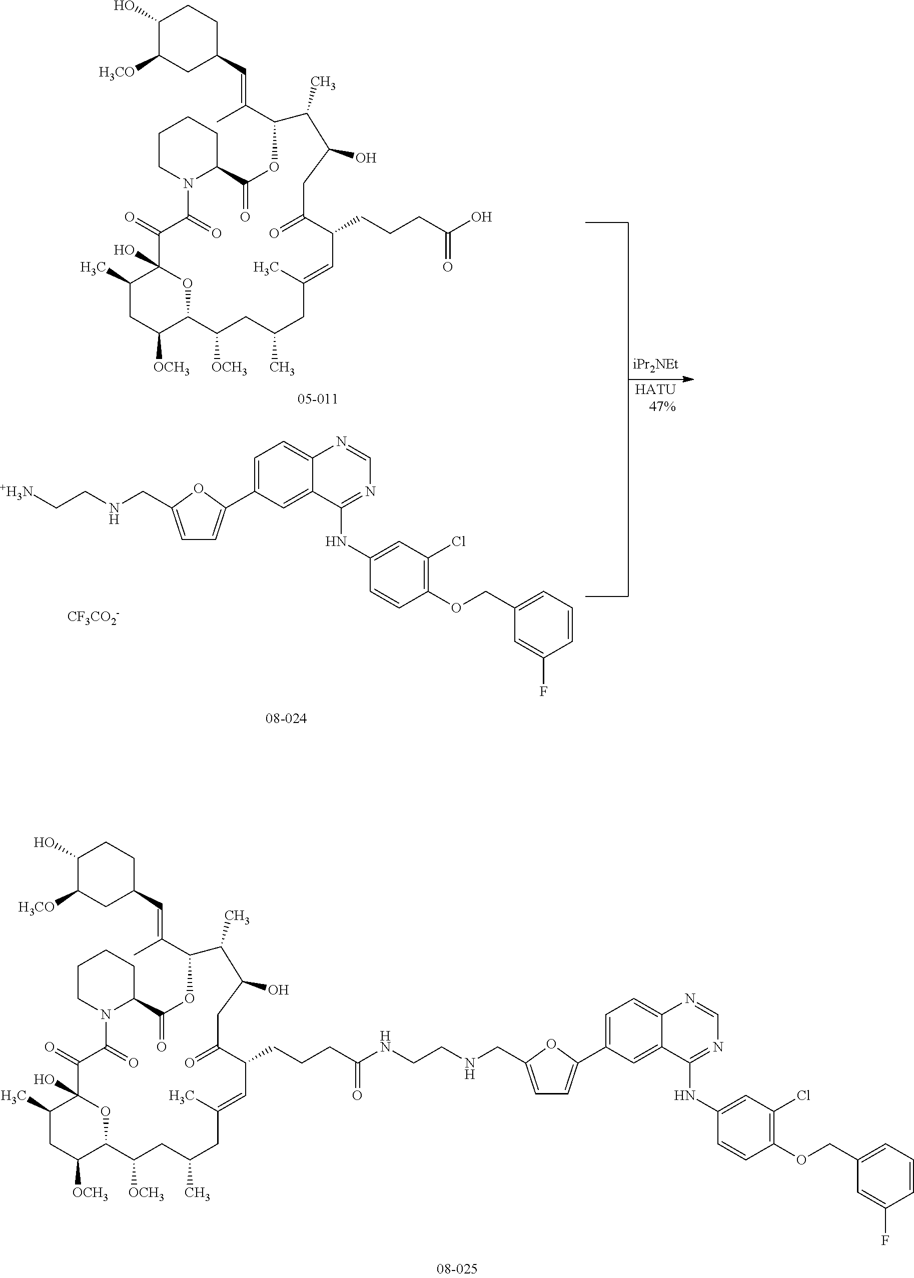 [SMILES: CO[C@H]1C[C@@H](C)C/C(C)=C/[C@@H](CCCC(=O)O)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; "iPr2NEt" ; [NH3+]CCNCc1ccc(-c2ccc3ncnc(Nc4ccc(OCc5cccc(F)c5)c(Cl)c4)c3c2)o1 ; "CF3CO2-" ; CO[C@H]1C[C@@H](C)C/C(C)=C/[C@@H](CCCC(=O)NCCNCc2ccc(-c3ccc4ncnc(Nc5ccc(OCc6cccc(F)c6)c(Cl)c5)c4c3)o2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

An oven-dried 1-dram vial was charged 05-020 (20 mg, 0.024 mmol), 08-024 (15 mg, 0.024 mmol), DMF (0.12 mL) and a magnetic stir bar. N,N-Diisopropylethylamine (12.3 μL, 0.071 mmol) was added and the mixture was stirred until all reactants had gone into solution. HATU (10.7 mg, 0.0282 mmol) was added as a 10% solution in DMF. The resulting solution was stirred at 23° C. and the reaction progress was monitored by LC-MS. In 15 min, LC-MS showed full consumption of the amine starting material. The reaction mixture was diluted with 50% acetonitrile-water to a volume of 4.2 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a yellow solid (15 mg, 47%). HRMS (ESI): Calcd for (C₇₃H₉₄ClFN₆O₁₅+2H)²+: 675.3303, Found: 675.3334.

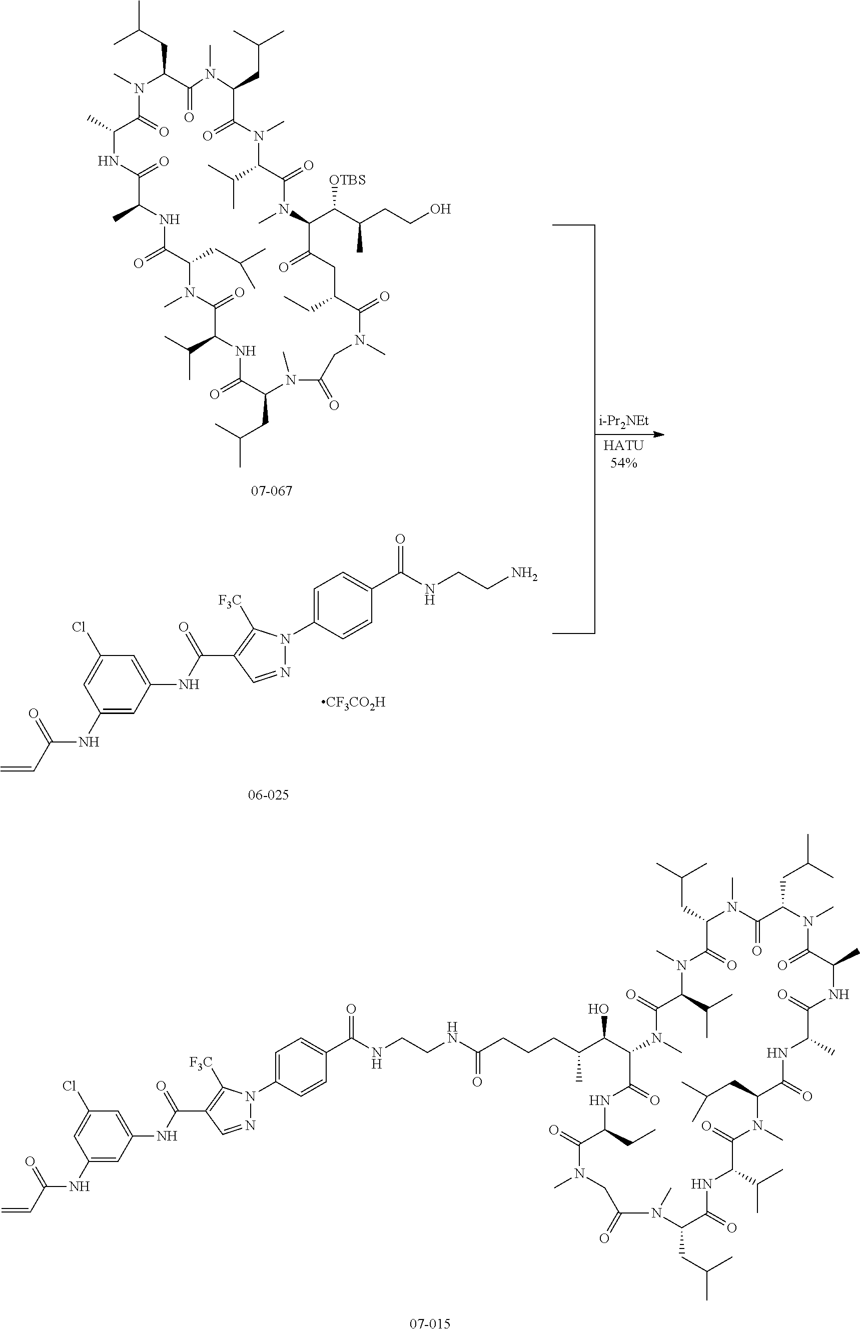

An oven-dried 1-dram vial was charged 06-067 (19.5 mg, 0.016 mmol), 06-025 (10 mg, 0.016 mmol), DMF (0.10 mL) and a magnetic stir bar. N,N-Diisopropylethylamine (8.2 μL, 0.047 mmol) and HATU (7.8 mg, 0.020 mmol) were added sequentially to the reaction mixture at 23° C. In 2 h, LC-MS showed full conversion of the starting material. The reaction mixture was diluted with 500% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.100 formic acid, 40 min, 20 mL/min) to afford the product as a white solid (13.6 mg, 54%). HRMS (ESI): Calcd for (C₈₅H₁₂₉ClF₃N₁₇O₁₆−H)⁻: 1734.9366, Found: 1734.9539.

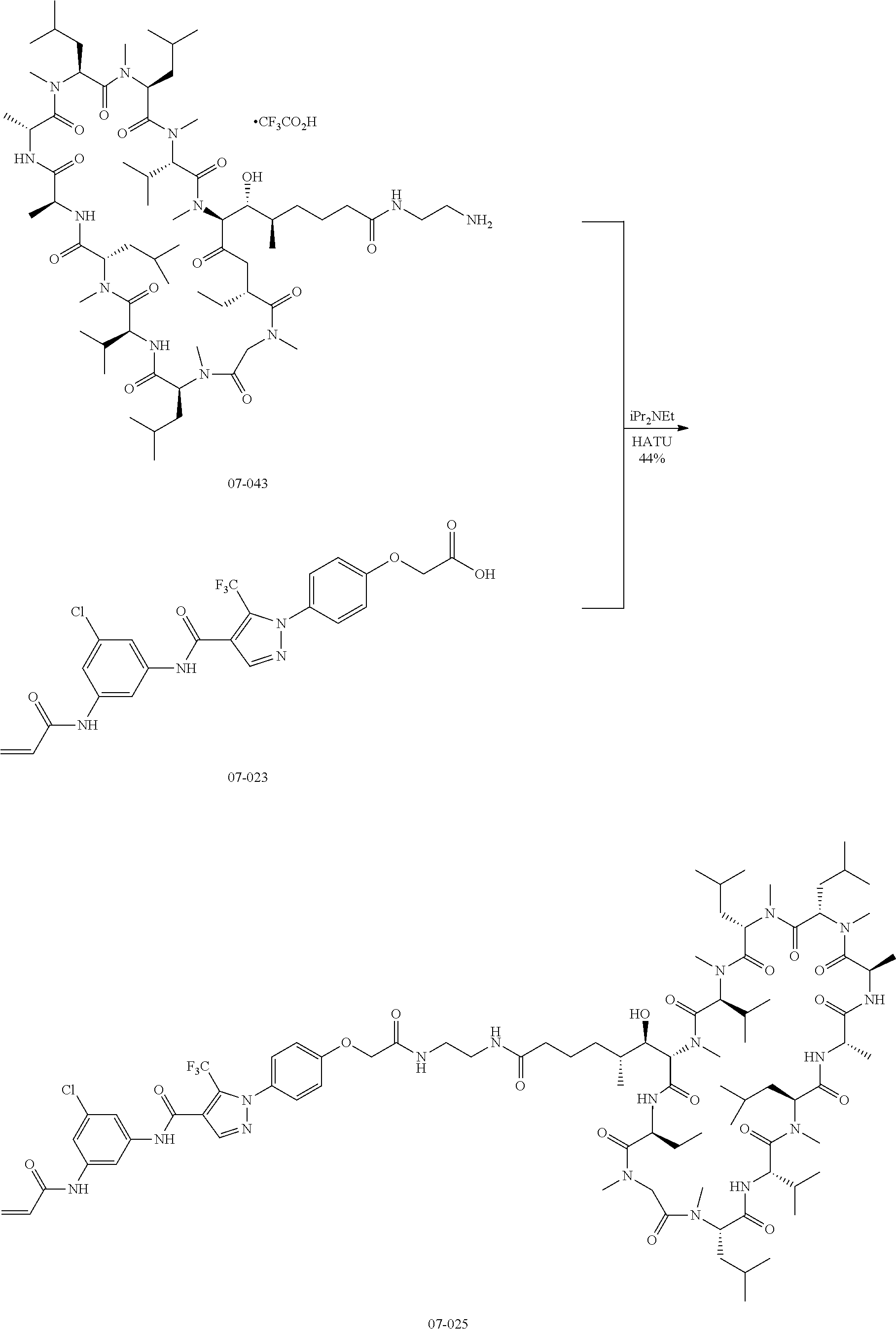

An oven-dried 1-dram vial was charged 07-043 (15 mg, 0.011 mmol), 07-023 (5.7 mg, 0.011 mmol), DMF (0.11 mL) and a magnetic stir bar. N,N-Diisopropylethylamine (5.9 μL, 0.034 mmol) and HATU (5.2 mg, 0.014 mmol) were added sequentially to the reaction mixture at 23° C. In 2 h, LC-MS showed full conversion of the starting material. The reaction mixture was diluted with 500% acetonitrile-water to a volume of 5.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (8.9 mg, 440%). HRMS (ESI): Calcd for (C₈₆H₁₃₁ClF₃N₁₇O₁₇−H)⁻: 1764.9471, Found: 1764.9409.

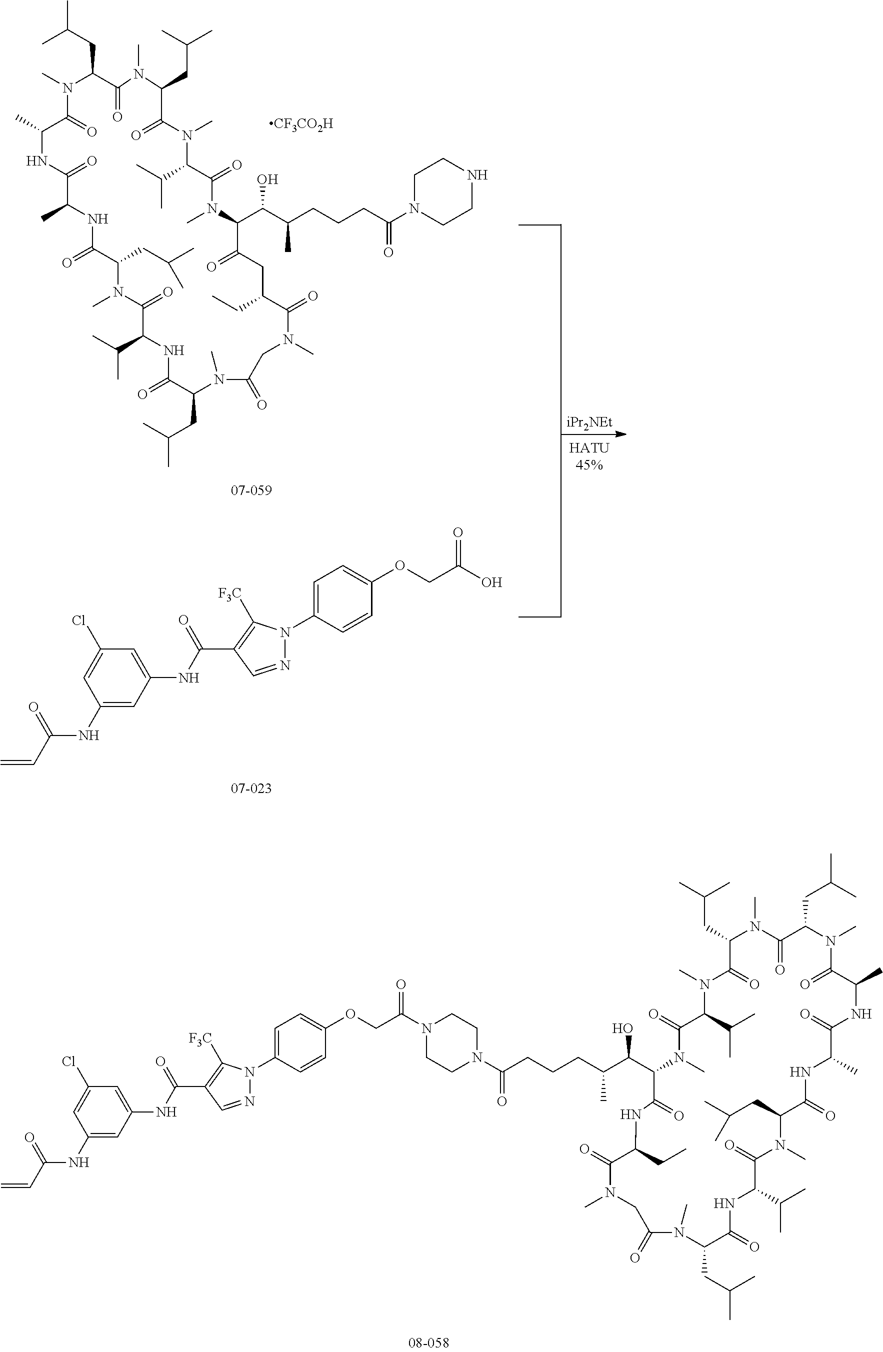

A mixture of 07-059 (12 mg, 0.0085 mmol) and 07-023 (4.7 mg, 0.0093 mmol) was dried by azeotropic evaporation of their suspension in benzene (1 mL). The residue was dissolved in DMF (0.20 mL). N,N-Diisopropylethylamine (8.4 μL, 0.048 mmol) and HATU (3.5 mg, 0.0093 mmol) were added sequentially to the solution, and the mixture was stirred at 23° C. while the reaction progress was monitored by LC-MS. In 1 h, LC-MS showed full consumption of the 07-059 starting material. The residue was diluted with 50% acetonitrile-water to a volume of 4.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (6.9 mg, 45%). HRMS (ESI): Calcd for (C₈₈H₁₃₃ClF₃N₁₇O₁₇+2H)²⁺: 896.9931, Found: 896.9883.

### Example 6: Analogs

TABLE 2
EGFR Analogs

| Compound | Structure |
|---|---|
| 07-057 | 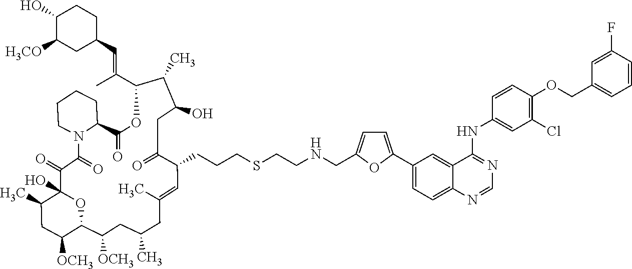 [SMILES: CO[C@H]1C[C@@H](C)C/C(C)=C/[C@@H](CCCSCCNCc2ccc(-c3ccc4ncnc(Nc5ccc(OCc6cccc(F)c6)c(Cl)c5)c4c3)o2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C] |
| 07-058 | 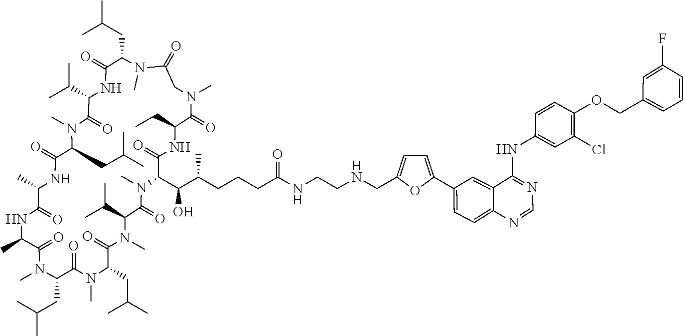 [SMILES: CC[C@@H]1NC(=O)[C@H]([C@H](O)[C@H](C)CCCC(=O)NCCNCc2ccc(-c3ccc4ncnc(Nc5ccc(OCc6cccc(F)c6)c(Cl)c5)c4c3)o2)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O] |
| 08-025 | 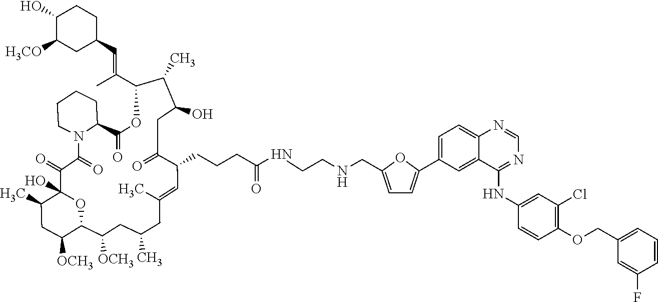 [SMILES: CO[C@H]1C[C@@H](C)C/C(C)=C/[C@@H](CCCC(=O)NCCNCc2ccc(-c3ccc4ncnc(Nc5ccc(OCc6cccc(F)c6)c(Cl)c5)c4c3)o2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C] |
| 08-047 | 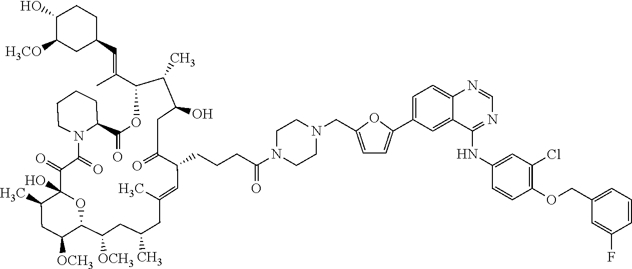 [SMILES: CO[C@H]1C[C@@H](C)C/C(C)=C/[C@@H](CCCC(=O)N2CCN(Cc3ccc(-c4ccc5ncnc(Nc6ccc(OCc7cccc(F)c7)c(Cl)c6)c5c4)o3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C] |
| 08-068 | 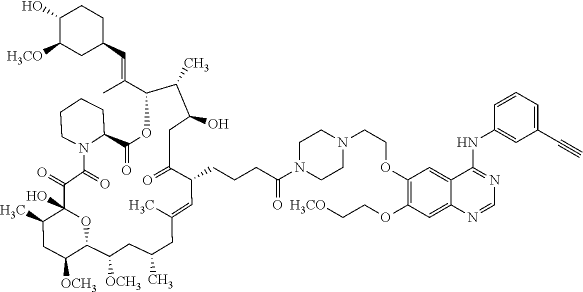 [SMILES: C#Cc1cccc(Nc2ncnc3cc(OCCOC)c(OCCN4CCN(C(=O)CCC[C@@H]5/C=C(\C)C[C@H](C)C[C@H](OC)[C@H]6O[C@@](O)(C(=O)C(=O)N7CCCC[C@H]7C(=O)O[C@H](/C(C)=C/[C@@H]7CC[C@@H](O)[C@H](OC)C7)[C@H](C)[C@@H](O)CC5=O)[C@H](C)C[C@@H]6OC)CC4)cc23)c1] |
| 08-069 | 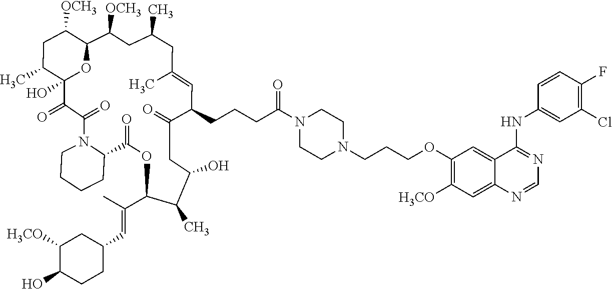 [SMILES: COc1cc2ncnc(Nc3ccc(F)c(Cl)c3)c2cc1OCCCN1CCN(C(=O)CCC[C@@H]2/C=C(\C)C[C@H](C)C[C@H](OC)[C@H]3O[C@@](O)(C(=O)C(=O)N4CCCC[C@H]4C(=O)O[C@H](/C(C)=C/[C@@H]4CC[C@@H](O)[C@H](OC)C4)[C@H](C)[C@@H](O)CC2=O)[C@H](C)C[C@@H]3OC)CC1] |

TABLE 3
LRRK Analogs

| Compound | Structure |
|---|---|
| 08-074 | 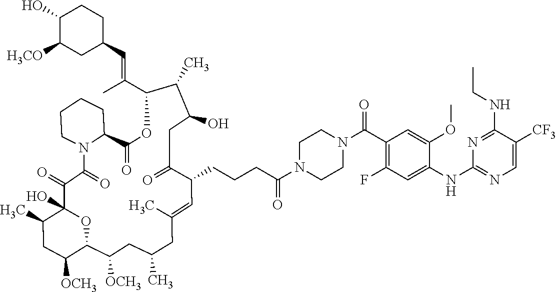 [SMILES: CCNc1nc(Nc2cc(F)c(C(=O)N3CCN(C(=O)CCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)CC3)cc2OC)ncc1C(F)(F)F] |

TABLE 4
KRAS Analogs

| Compound | Structure |
|---|---|
| 06-027 | 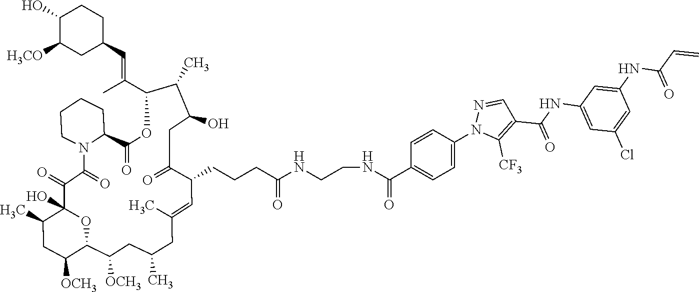 [SMILES: C=CC(=O)Nc1cc(Cl)cc(NC(=O)c2cnn(-c3ccc(C(=O)NCCNC(=O)CCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)cc3)c2C(F)(F)F)c1] |
| 07-015 | 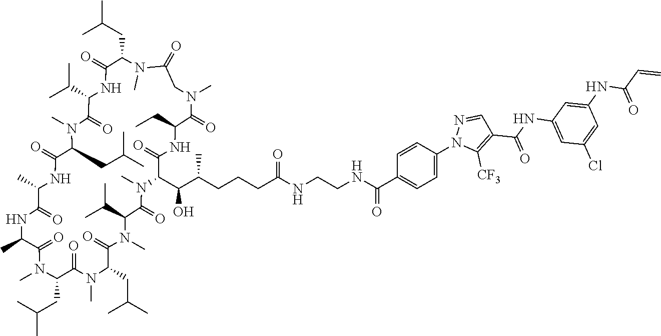 [SMILES: C=CC(=O)Nc1cc(Cl)cc(NC(=O)c2cnn(-c3ccc(C(=O)NCCNC(=O)CCC[C@@H](C)[C@@H](O)[C@H]4C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N4C)cc3)c2C(F)(F)F)c1] |
| 07-025 | 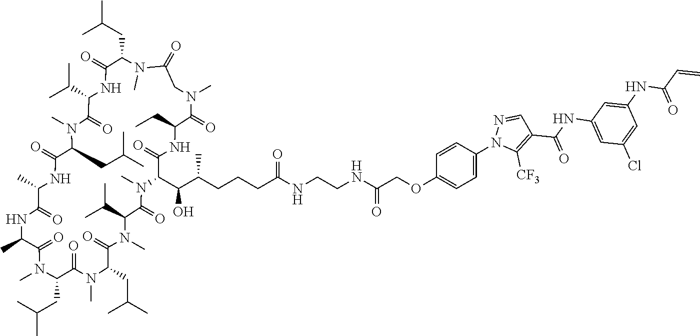 [SMILES: C=CC(=O)Nc1cc(Cl)cc(NC(=O)c2cnn(-c3ccc(OCC(=O)NCCNC(=O)CCC[C@@H](C)[C@@H](O)[C@H]4C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N4C)cc3)c2C(F)(F)F)c1] |
| 08-027 | 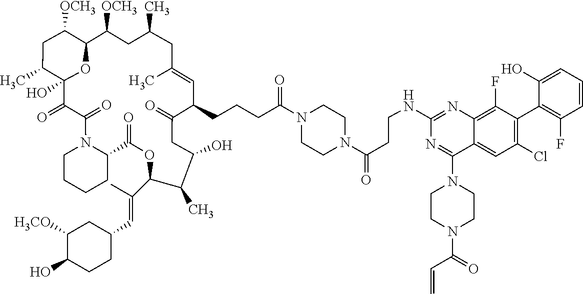 [SMILES: C=CC(=O)N1CCN(c2nc(NCCC(=O)N3CCN(C(=O)CCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)CC3)nc3c(F)c(-c4c(O)cccc4F)c(Cl)cc23)CC1] |
| 08-028 | 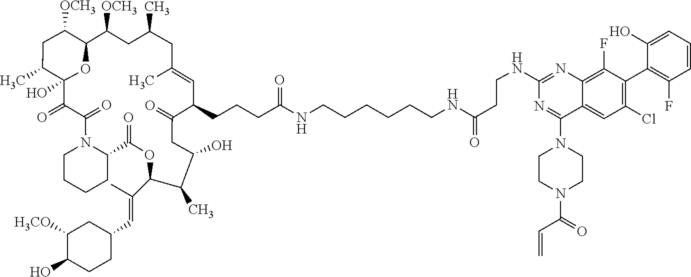 [SMILES: C=CC(=O)N1CCN(c2nc(NCCC(=O)NCCCCCCNC(=O)CCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/[C@@H]5CC[C@@H](O)[C@H](OC)C5)[C@H](C)[C@@H](O)CC3=O)[C@H](C)C[C@@H]4OC)nc3c(F)c(-c4c(O)cccc4F)c(Cl)cc23)CC1] |
| 08-057 | 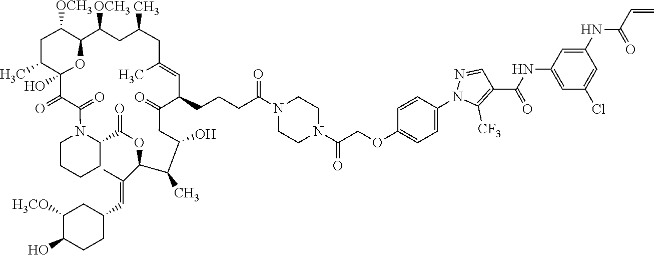 [SMILES: C=CC(=O)Nc1cc(Cl)cc(NC(=O)c2cnn(-c3ccc(OCC(=O)N4CCN(C(=O)CCC[C@@H]5/C=C(\C)C[C@H](C)C[C@H](OC)[C@H]6O[C@@](O)(C(=O)C(=O)N7CCCC[C@H]7C(=O)O[C@H](/C(C)=C/[C@@H]7CC[C@@H](O)[C@H](OC)C7)[C@H](C)[C@@H](O)CC5=O)[C@H](C)C[C@@H]6OC)CC4)cc3)c2C(F)(F)F)c1] |
| 08-058 | 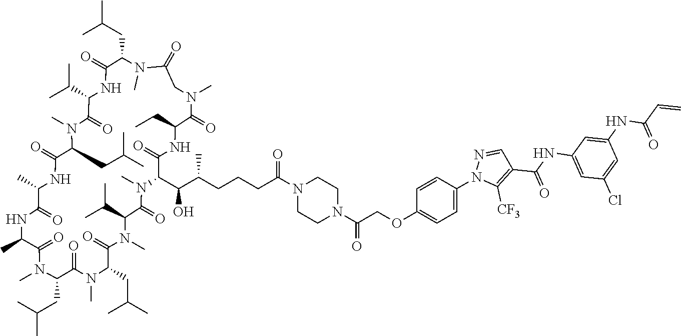 [SMILES: C=CC(=O)Nc1cc(Cl)cc(NC(=O)c2cnn(-c3ccc(OCC(=O)N4CCN(C(=O)CCC[C@@H](C)[C@@H](O)[C@H]5C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N5C)CC4)cc3)c2C(F)(F)F)c1] |
| 05-042, 07-028 | 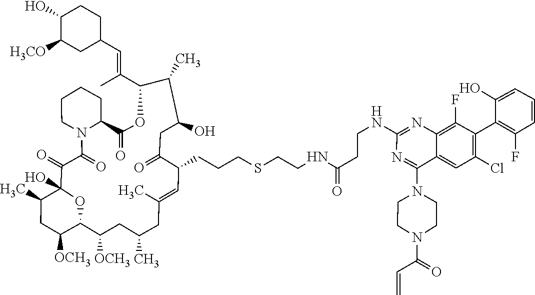 [SMILES: C=CC(=O)N1CCN(c2nc(NCCC(=O)NCCSCCC[C@@H]3/C=C(\C)C[C@H](C)C[C@H](OC)[C@H]4O[C@@](O)(C(=O)C(=O)N5CCCC[C@H]5C(=O)O[C@H](/C(C)=C/C5CC[C@@H](O)[C@H](OC)C5)[C@H](C)[C@@H](O)CC3=O)[C@H](C)C[C@@H]4OC)nc3c(F)c(-c4c(O)cccc4F)c(Cl)cc23)CC1] |
| 07-014 | 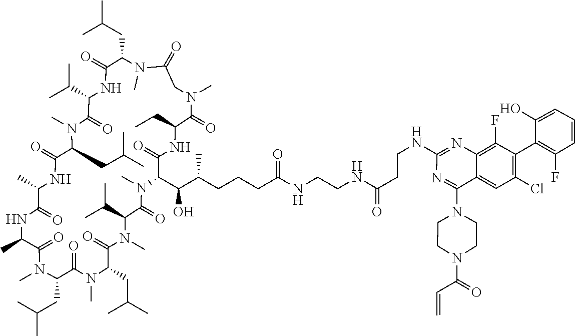 [SMILES: C=CC(=O)N1CCN(c2nc(NCCC(=O)NCCNC(=O)CCC[C@@H](C)[C@@H](O)[C@H]3C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N3C)nc3c(F)c(-c4c(O)cccc4F)c(Cl)cc23)CC1] |
| 07-079 | 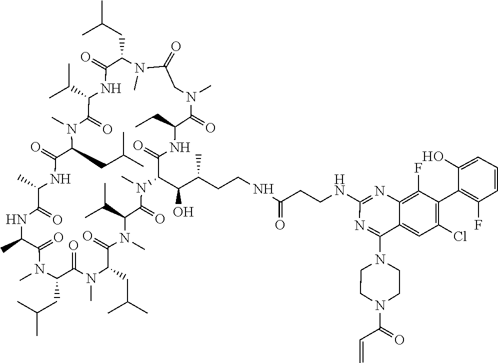 [SMILES: C=CC(=O)N1CCN(c2nc(NCCC(=O)NCC[C@@H](C)[C@@H](O)[C@H]3C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N3C)nc3c(F)c(-c4c(O)cccc4F)c(Cl)cc23)CC1] |
| 07-089 | 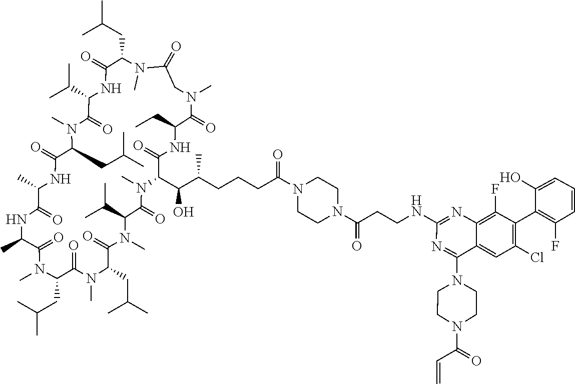 [SMILES: C=CC(=O)N1CCN(c2nc(NCCC(=O)N3CCN(C(=O)CCC[C@@H](C)[C@@H](O)[C@H]4C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N4C)CC3)nc3c(F)c(-c4c(O)cccc4F)c(Cl)cc23)CC1] |
| 07-090 | 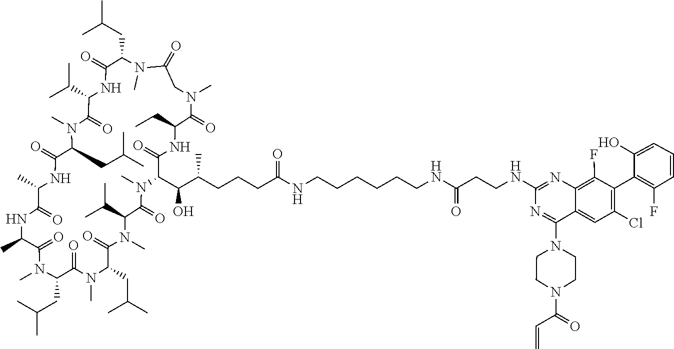 [SMILES: C=CC(=O)N1CCN(c2nc(NCCC(=O)NCCCCCCNC(=O)CCC[C@@H](C)[C@@H](O)[C@H]3C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N3C)nc3c(F)c(-c4c(O)cccc4F)c(Cl)cc23)CC1] |

TABLE 5
Cyclosporin Analogs

| Compound | Structure |
|---|---|
| 06-082 | 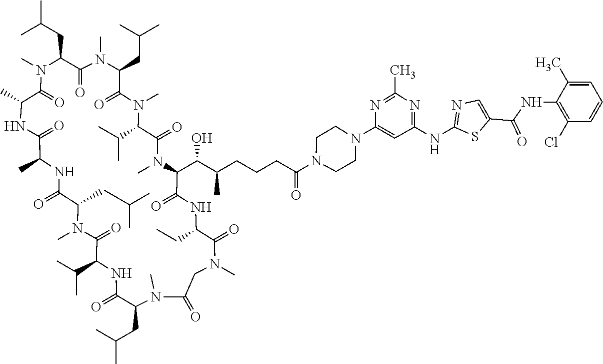 [SMILES: CC[C@@H]1NC(=O)[C@H]([C@H](O)[C@H](C)CCCC(=O)N2CCN(c3cc(Nc4ncc(C(=O)Nc5c(C)cccc5Cl)s4)nc(C)n3)CC2)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O] |
| 06-083 | 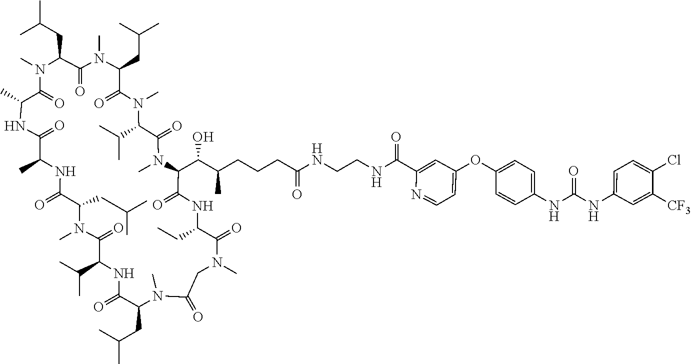 [SMILES: CC[C@@H]1NC(=O)[C@H]([C@H](O)[C@H](C)CCCC(=O)NCCNC(=O)c2cc(Oc3ccc(NC(=O)Nc4ccc(Cl)c(C(F)(F)F)c4)cc3)ccn2)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O] |

TABLE 6
Additional Analogs

| Compound | Structure |
|---|---|
| 05-016 | 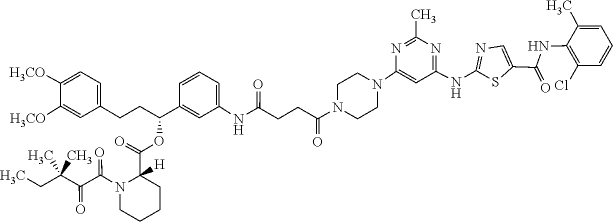 |
| 05-049 | 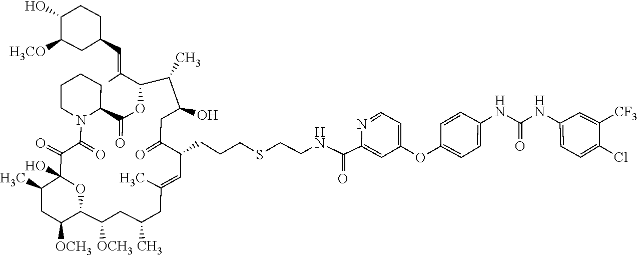 [SMILES: CO[C@H]1C[C@@H](C)C/C(C)=C/[C@@H](CCCSCCNC(=O)c2cc(Oc3ccc(NC(=O)Nc4ccc(Cl)c(C(F)(F)F)c4)cc3)ccn2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C] |
| 07-026 | 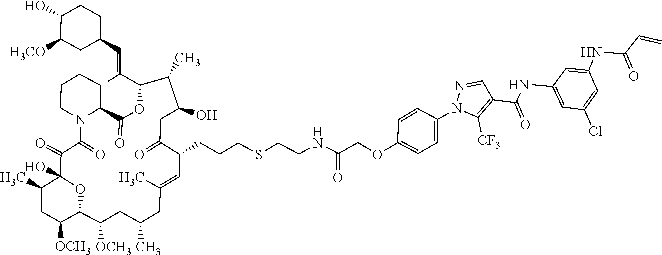 [SMILES: C=CC(=O)Nc1cc(Cl)cc(NC(=O)c2cnn(-c3ccc(OCC(=O)NCCSCCC[C@@H]4/C=C(\C)C[C@H](C)C[C@H](OC)[C@H]5O[C@@](O)(C(=O)C(=O)N6CCCC[C@H]6C(=O)O[C@H](/C(C)=C/[C@@H]6CC[C@@H](O)[C@H](OC)C6)[C@H](C)[C@@H](O)CC4=O)[C@H](C)C[C@@H]5OC)cc3)c2C(F)(F)F)c1] |
| 05-022 | 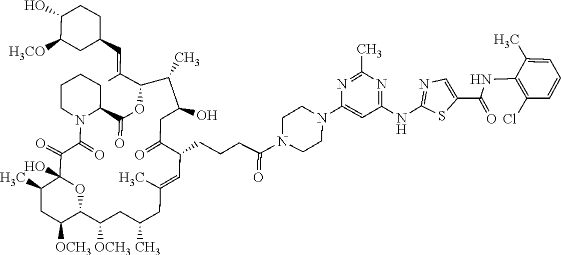 [SMILES: CO[C@H]1C[C@@H](C)C/C(C)=C/[C@@H](CCCC(=O)N2CCN(c3cc(Nc4ncc(C(=O)Nc5c(C)cccc5Cl)s4)nc(C)n3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C] |

### Example 7: HGK Inhibitors

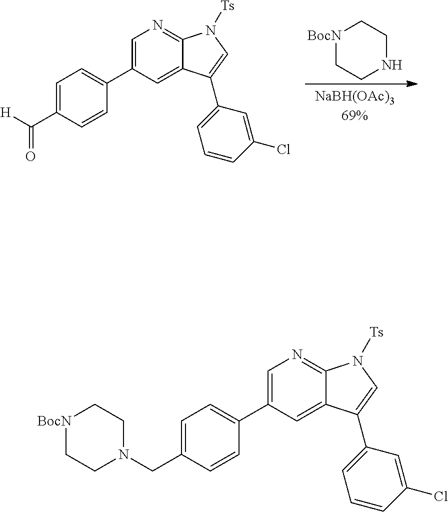 [SMILES: Cc1ccc(S(=O)(=O)n2cc(-c3cccc(Cl)c3)c3cc(-c4ccc(C=O)cc4)cnc32)cc1 ; CC(C)(C)OC(=O)N1CCNCC1 ; Cc1ccc(S(=O)(=O)n2cc(-c3cccc(Cl)c3)c3cc(-c4ccc(CN5CCN(C(=O)OC(C)(C)C)CC5)cc4)cnc32)cc1]

N-Boc-Piperazine (229 mg, 1.23 mmol) and sodium triacetoxyborohydride (196 mg, 0.924 mmol) were added sequentially to a stirred solution of 4-[3-(3-chlorophenyl)-1-(p-tolylsulfonyl)pyrrolo[2,3-b]pyridin-5-yl]benzaldehyde (44) (300 mg, 0.6161 mmol) in DCM (3.0803 mL) at 23° C. The resulting mixture was stirred at 23° C. and the reaction progress was monitored by LC-MS. In 18 h, LC-MS analysis showed full consumption of the aldehyde starting material. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (20-50% ethyl acetate-hexanes, 4-g RediSep(R) Rf column, Teledyne ISCO, Lincoln, Nebr.) to afford the product as a yellow powder (279 mg, 69%). ¹H NMR (400 MHz, CDCl₃) δ 8.72 (d, J=2.1 Hz, 1H), 8.23-8.13 (m, 3H), 7.94 (s, 1H), 7.62 (t, J=1.8 Hz, 1H), 7.59-7.49 (m, 3H), 7.47-7.42 (m, 2H), 7.43-7.30 (m, 4H), 3.58 (s, 2H), 3.55-3.43 (m, 4H), 2.50-2.34 (m, 7H), 1.48 (s, 9H). HRMS (ESI): Calcd for (C₃₆H₃₇ClN₄O₄S+H)⁺: 657.2302, Found: 657 2278.

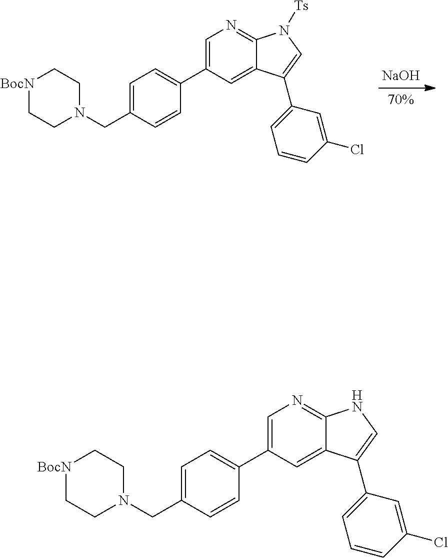 [SMILES: Cc1ccc(S(=O)(=O)n2cc(-c3cccc(Cl)c3)c3cc(-c4ccc(CN5CCN(C(=O)OC(C)(C)C)CC5)cc4)cnc32)cc1 ; "NaOH" ; CC(C)(C)OC(=O)N1CCN(Cc2ccc(-c3cnc4[nH]cc(-c5cccc(Cl)c5)c4c3)cc2)CC1]

tert-butyl 4-[[4-[3-(3-chlorophenyl)-1-(p-tolylsulfonyl)pyrrolo[2,3-b]pyridin-5-yl]phenyl]methyl]piperazine-1-carboxylate (279 mg, 0.43 mmol) was dissolved in a 1:1:1 mixture of acetone (2 mL):methanol (2 mL): 2 M aqueous NaOH (2 mL). The mixture was heated to 65° C. In 1 h, LC-MS analysis showed full deprotection of the tosyl group. The reaction mixture was partitioned between ethyl acetate (10 mL) and 1 N NaOH (10 mL). The aqueous layer was extracted with ethyl acetate (2×10 mL). The combined organic layers were dried over sodium sulfate, and the dried solution was concentrated. The residue was purified by column chromatography (20-50% ethyl acetate-hexanes, 4-g RediSep(R) Rf column, Teledyne ISCO, Lincoln, Nebr.) to afford the product as a yellow powder (150 mg, 70%). ¹H NMR (400 MHz, CDCl₃) δ 9.43 (s, 1H), 8.64 (d, J=2.1 Hz, 1H), 8.38 (d, J=2.0 Hz, 1H), 7.70-7.55 (m, 5H), 7.50-7.37 (m, 3H), 7.32 (ddd, J=8.0, 2.1, 1.1 Hz, 1H), 3.61 (s, 2H), 3.48 (t, J=5.1 Hz, 4H), 2.46 (t, J=5.1 Hz, 4H), 1.49 (s, 9H). HRMS (ESI): Calcd for (C₂₉H₃₁ClN₄O₂+H)⁺: 503.2214, Found: 503.2233.

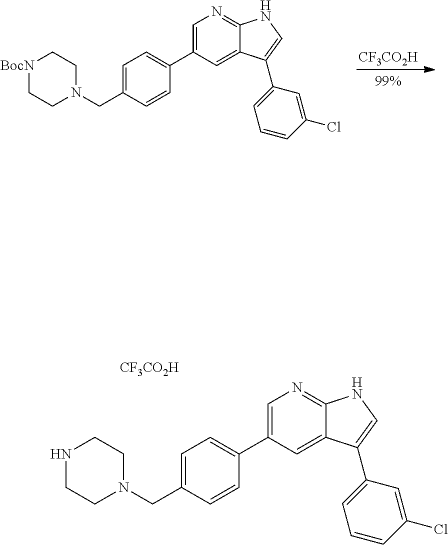 [SMILES: CC(C)(C)OC(=O)N1CCN(Cc2ccc(-c3cnc4[nH]cc(-c5cccc(Cl)c5)c4c3)cc2)CC1 ; "CF3CO2H" ; Clc1cccc(-c2c[nH]c3ncc(-c4ccc(CN5CCNCC5)cc4)cc23)c1 ; "CF3CO2H"]

tert-butyl 4-[[4-[3-(3-chlorophenyl)-1H-pyrrolo[2,3-b]pyridin-5-yl]phenyl]methyl]piperazine-1-carboxylate (150 mg, 0.299 mmol) was dissolved in 50% trifluoroacetic acid-dichloromethane (2.0 mL) and the resulting solution was allowed to stand at 23° C. for 1 h. At this point, LC-MS analysis showed full consumption of the starting material and formation of the desired product. The reaction mixture was concentrated under reduced pressure and the residue was triturated with ether and dried under vacuum to afford the product as a yellow solid (151 mg, 99%). ¹H NMR (400 MHz, CDCl₃) δ 9.43 (s, 1H), 8.64 (d, J=2.1 Hz, 1H), 8.38 (d, J=2.0 Hz, 1H), 7.70-7.55 (m, 5H), 7.50-7.37 (m, 3H), 7.32 (ddd, J=8.0, 2.1, 1.1 Hz, 1H), 3.61 (s, 2H), 3.48 (t, J=5.1 Hz, 4H), 2.46 (t, J=5.1 Hz, 4H), 1.49 (s, 9H). HRMS (ESI): Calcd for (C₂₄H₂₃ClN₄+H)⁺: 403.1689, Found: 403.1698.

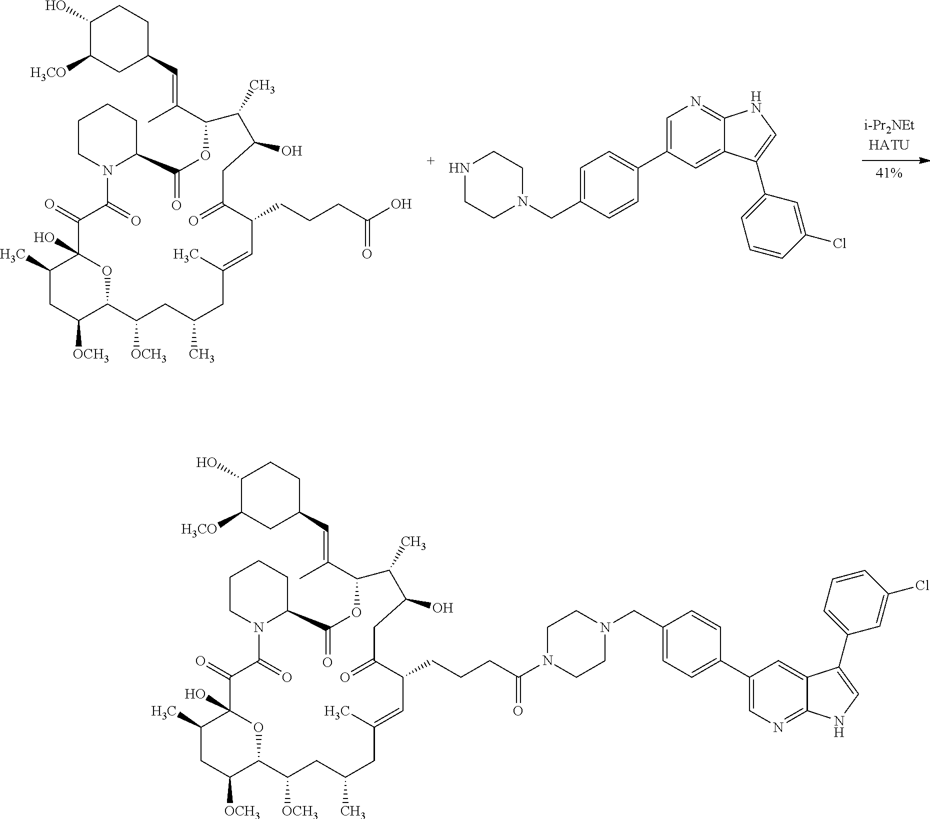 [SMILES: CO[C@H]1C[C@@H](C)C/C(C)=C/[C@@H](CCCC(=O)O)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C ; Clc1cccc(-c2c[nH]c3ncc(-c4ccc(CN5CCNCC5)cc4)cc23)c1 ; CO[C@H]1C[C@@H](C)C/C(C)=C/[C@@H](CCCC(=O)N2CCN(Cc3ccc(-c4cnc5[nH]cc(-c6cccc(Cl)c6)c5c4)cc3)CC2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

N,N-Diisopropylethylamine (6.2 μL, 0.035 mmol) and HATU (4.5 mg, 0.012 mmol) were added sequentially to a stirred solution of 3-(3-chlorophenyl)-5-[4-(piperazin-1-ylmethyl)phenyl]-1H-pyrrolo[2,3-b]pyridine trifluoroacetic acid salt (6.1 mg, 0.012 mmol) and FK506-C4-Acid (10 mg, 0.012 mmol) in DMF (0.2 mL) at 23° C. The resulting mixture quickly turned yellow, and LC-MS analysis at 15 min showed full consumption of the FK506 acid starting material. The reaction mixture was diluted with 50% acetonitrile-water to a volume of 3.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (5.9 mg, 41%). HRMS (ESI): Calcd for (C₆₉H₉₂ClN₅O₁₃+2H)²⁺: 617.8268, Found: 617.8257.

### REFERENCES

Briesewitz, R., Ray, G. T., Wandless, T. J. & Crabtree, G. R. Affinity modulation of small-molecule ligands by borrowing endogenous protein surfaces. Proc. Natl. Acad. Sci. U.S.A 96, 1953-8 (1999).
Modulating a pharmacokinetic property of a drug by administering a bifunctional molecule containing the drug. US 20050209265, U.S. Pat. No. 6,887,842
Administering bifunctional molecules containing a drug moiety and presenter protein ligand for therapy. US20050209146
Bifunctional molecules and therapies based thereon. US20020147133
Targeted bifunctional molecules and therapies based thereon U.S. Pat. No. 7,498,025
Small Molecule Composite Surfaces As Inhibitors Of Protein-Protein Interactions. US 20160333054 Gestwicki, J. E. Harnessing Chaperones to Generate Small-Molecule Inhibitors of Amyloid “Aggregation. 865, 865-870 (2011).
Neurodegenerative protein aggregation inhibition methods and compounds. US20050209173
Marinec, P. S. et al. FK506-binding protein (FKBP) partitions a modified HIV protease inhibitor into blood cells and prolongs its lifetime in vivo. Proc. Natl. Acad. Sci. U.S.A 106, 1336-1341 (2009).
Marinec, P. S., Lancia, J. K. & Gestwicki, J. E. Bifunctional molecules evade cytochrome P450 metabolism by forming protective complexes with FK506-binding protein. Mol. Biosyst. 4, 571-578 (2008).
Dunyak, B. M., Nakamura, R. L., Frankel, A. D. & Gestwicki, J. E. Selective Targeting of Cells via Bispecific Molecules That Exploit Coexpression of Two Intracellular Proteins. ACS Chem. Biol. 10, 2441-2447 (2015).
Methods of screening bifunctional molecules for modulated pharmacokinetic properties US 20070054348
Pharmacokinetics of protease inhibitors and other drugs. US 20080306098
Braun, P. D. et al. A bifunctional molecule that displays context-dependent cellular activity. J. Am. Chem. Soc. 125, 7575-7580 (2003).
Sellmyer, M. A., Stankunas, K., Briesewitz, R., Crabtree, G. R. & Wandless, T. J. Engineering small molecule specificity in nearly identical cellular environments. Bioorganic Med. Chem. Lett. 17, 2703-2705 (2007).
Wu, X. et al. Creating diverse target-binding surfaces on FKBP12: Synthesis and evaluation of a rapamycin analogue library. ACS Comb. Sci. 13, 486-495 (2011).
Small Molecule Composite Surfaces As Inhibitors Of Protein-Protein Interactions. U.S. Pat. No. 9,260,484. US20160333054. US 20140200186
Guo, Z. et al. Rapamycin-inspired macrocycles with new target specificity. Nat. Chem. (2018). doi:10.1038/s41557-018-0187-4
Synthesis and composition of rapafucin libraries. WO2017136708A1.
Guo, Z.-F., Zhang, R. & Liang, F.-S. Facile functionalization of FK506 for biological studies by the thiol-ene ‘click’ reaction. RSC Adv. 4, 11400 (2014).
Marinec, P. S. et al. Synthesis of orthogonally reactive FK506 derivatives via olefin cross metathesis. Bioorganic Med. Chem. 17, 5763-5768 (2009).
Nambu, M. et al. A calcineurin antifungal strategy with analogs of FK506. Bioorganic Med. Chem. Lett. 27, 2465-2471 (2017).
Preparation of cyclosporin A analogs as Cyclophilin A inhibitors for treating dry eye and other conditions. PCT Int. Appl., 2013181339
Synthesis of cyclosporin derivatives for use in diagnosis or treatment of disease, plant protection, or pest control. PCT Int. Appl., 2013030208
Preparation of non-immunosuppressive cyclosporin A analogs modified on the (4R)-4-[(E)-2-butenyl]-4-methyl-L-threonine side chain by heterocyclic rings as potent cyclophilin A inhibitors for treating dry eye and other conditions. PCT Int. Appl., 2016160362
Preparation of non-immunosuppressive analogs of cyclosporin A derivatives possessing a (4R)-4-[(E)-2-butenyl]-4-methyl-L-threonine cyclized side-chain as potent inhibitors of cyclophilin D. PCT Int. Appl., 2016112321,
Preparation of cyclosporins for the treatment of immune disorders. U.S. Pat. Appl. Publ., 20040110666
Preparation of cyclosporins for the treatment of immune disorders. U.S. Pat. Appl. Publ., 20040157768
Preparation of cyclosporin alkyne analogs for preventing or treating viral-induced disorders. U.S. Pat. No. 7,696,165
Preparation of cyclosporin alkene analogues for preventing or treating viral-induced disorders. PCT Int. Appl., 2007112345
Preparation of cyclosporin alkyne/alkene analogues for preventing or treating viral-induced disorders. PCT Int. Appl., 2007112352
Preparation of cyclosporin alkynes as pharmaceutical agents. U.S. Pat. Appl. Publ., 20060074015
Preparation of cyclosporin alkyne analogues for preventing or treating viral-induced disorders. PCT Int. Appl., 2007112357
Preparation of cyclosporin analogues for pharmaceutical use. U.S. Pat. Appl. Publ., 20060069015,
Preparation of novel cyclosporins. PCT Int. Appl., 200408262
Preparation of cyclosporin A analogs modified at amino acid 1 and 3 as cyclophilin ligands with reduced immunosuppressivity for treating cyclophilin mediated diseases. PCT Int. Appl., 2012079172
A semisynthetic approach to olefinic analogs of amino acid one (MeBMT) in cyclosporin A. Tetrahedron Letters, 30(32), 4215-18; 1989
Semisynthetic di- and tri-functionalized non-immunosuppressive cyclosporin A derivatives as potential anti-HIV 1 drugs. Synlett, (2), 316-320; 2004
Preparation of novel cyclosporin A derivatives. Bioconjugate Chemistry, 3(1), 32-6; 1992
Dual Leucine Zipper Kinase Inhibitors for the Treatment of Neurodegeneration. J. Med. Chem., 2018, 61, 8078-8087.
Development of MAP4 Kinase Inhibitors as Motor Neuron-Protecting Agent. Cell Chemical Biology, 2019, 26, 1703-1715.
Design and Structural Characterization of Potent and Selective Inhibitors of Phosphatidylinositol 4 Kinase IIIβ. J. Med. Chem., 2016, 59 (5), 1830-1839.

1. A compound having the formula:
A-L¹-R¹;
wherein
A is an immunophilin-binding moiety;
L¹ is a bond or a covalent linker; and
R¹ is a kinase inhibitor, a pseudokinase inhibitor, a GTPase inhibitor, a histone-modifying enzyme inhibitor, or a monovalent anti-viral agent; wherein the compound is not

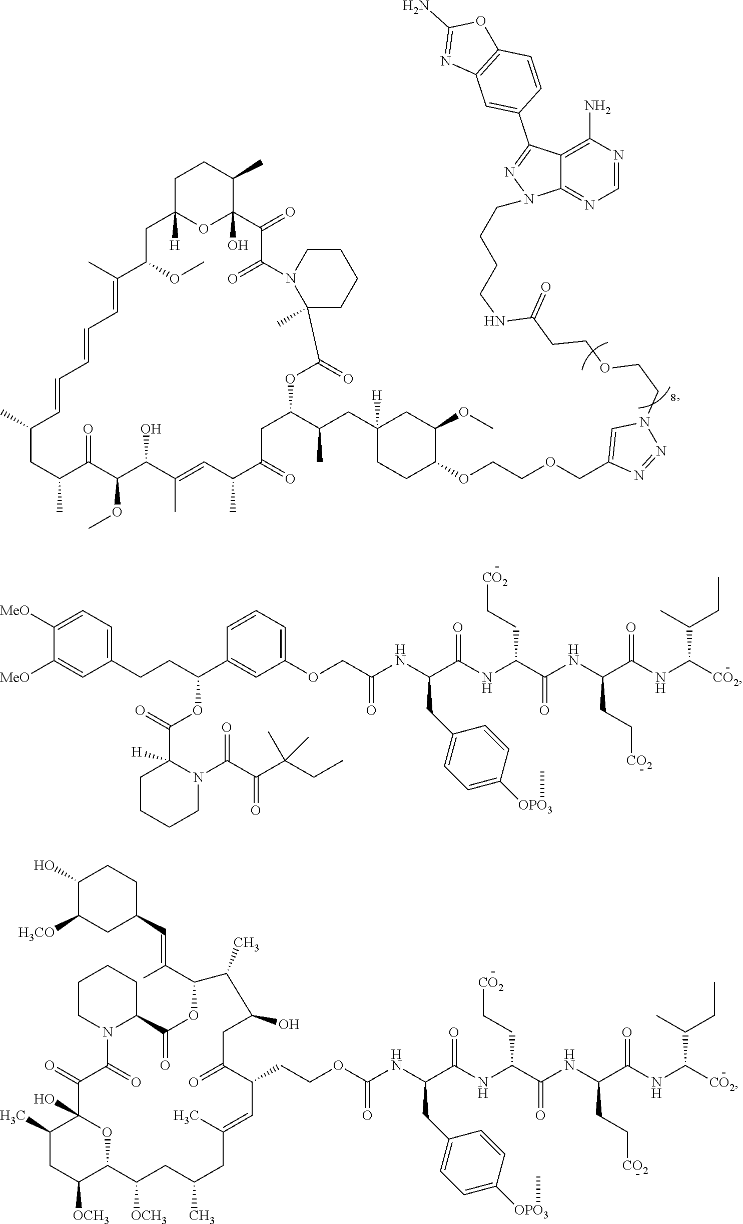

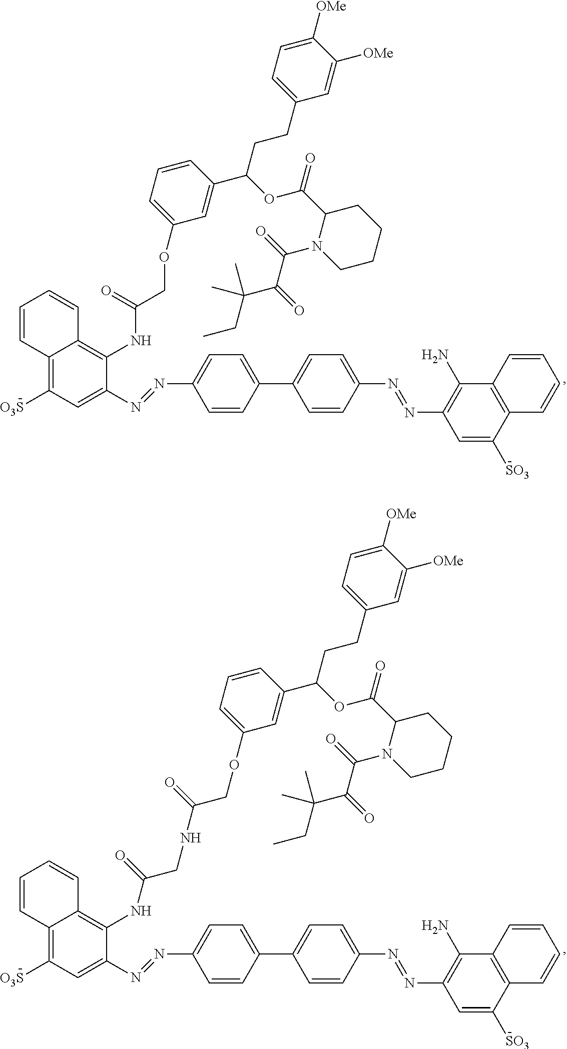

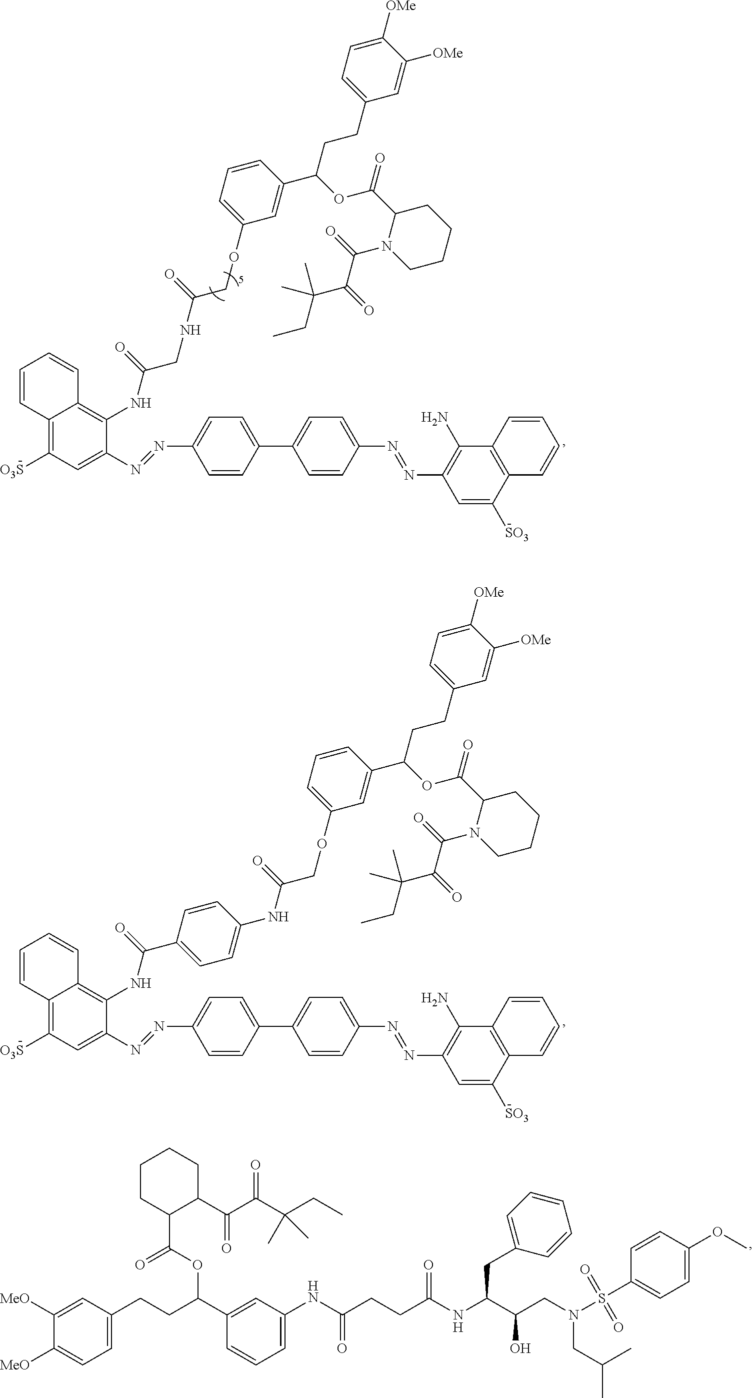

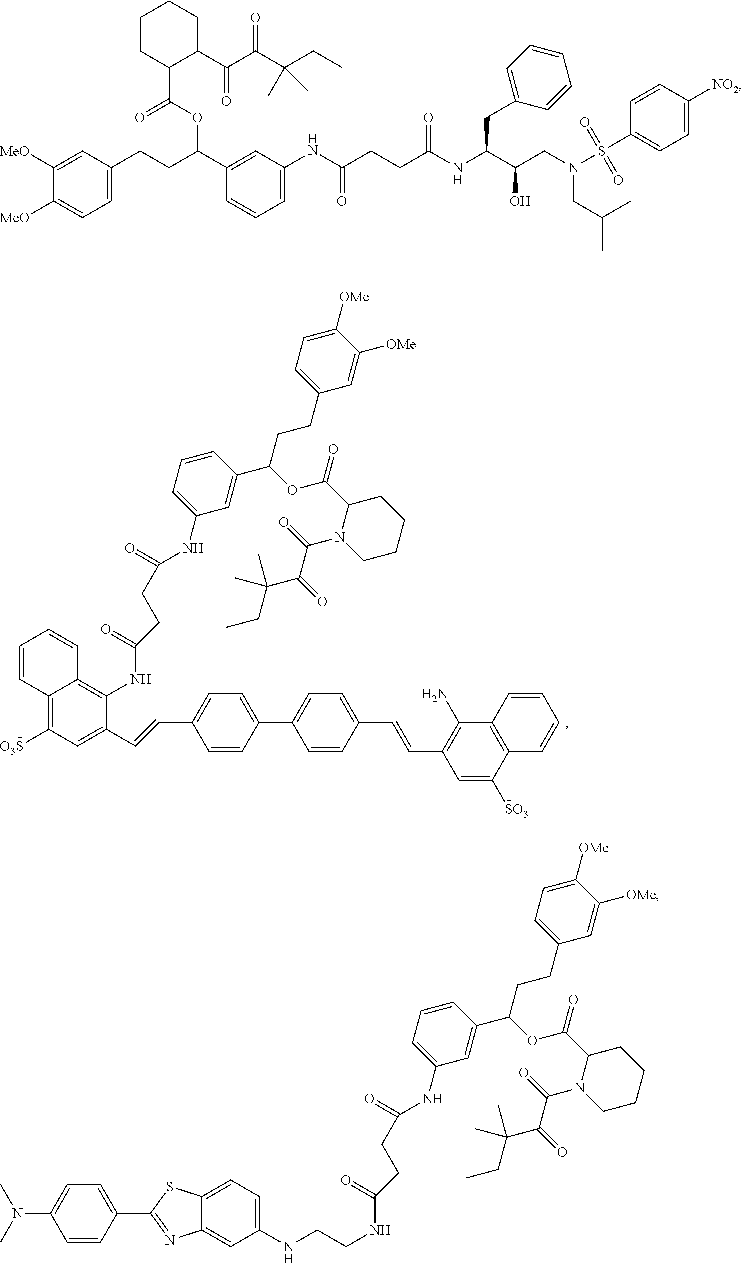

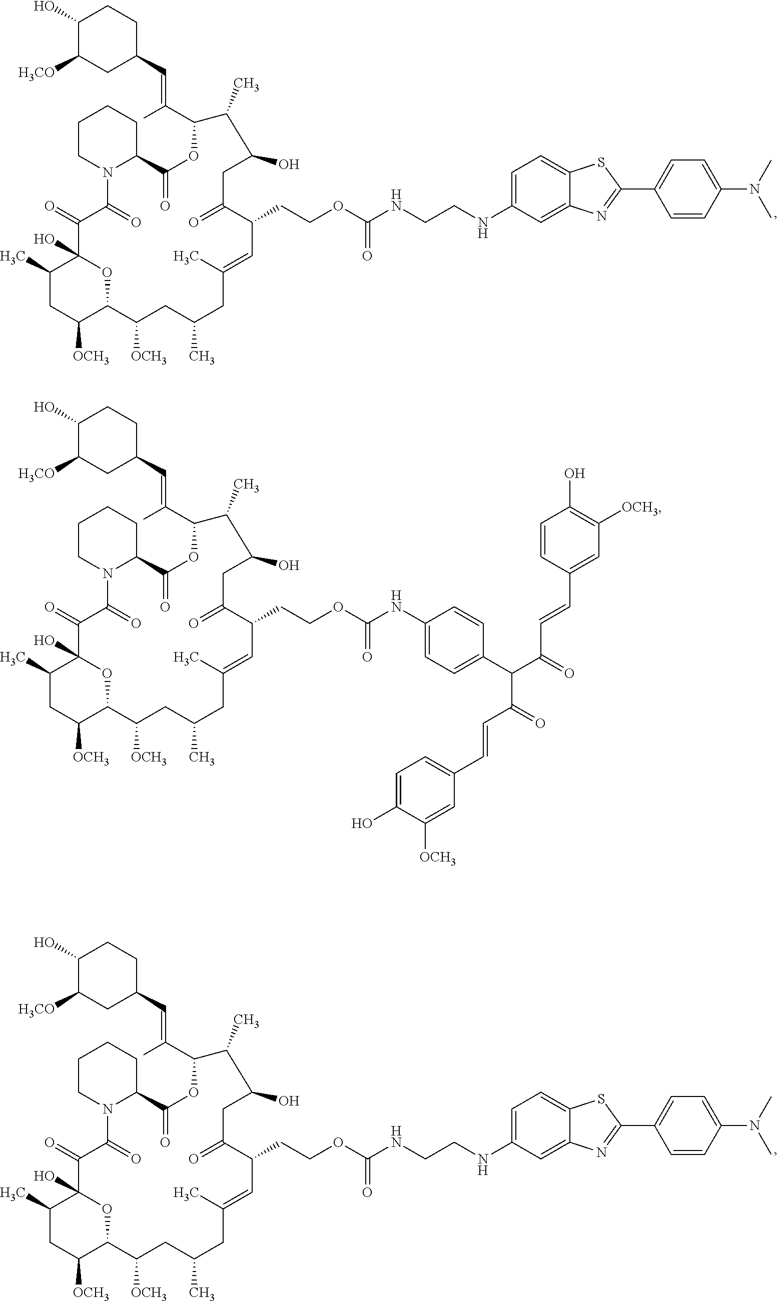

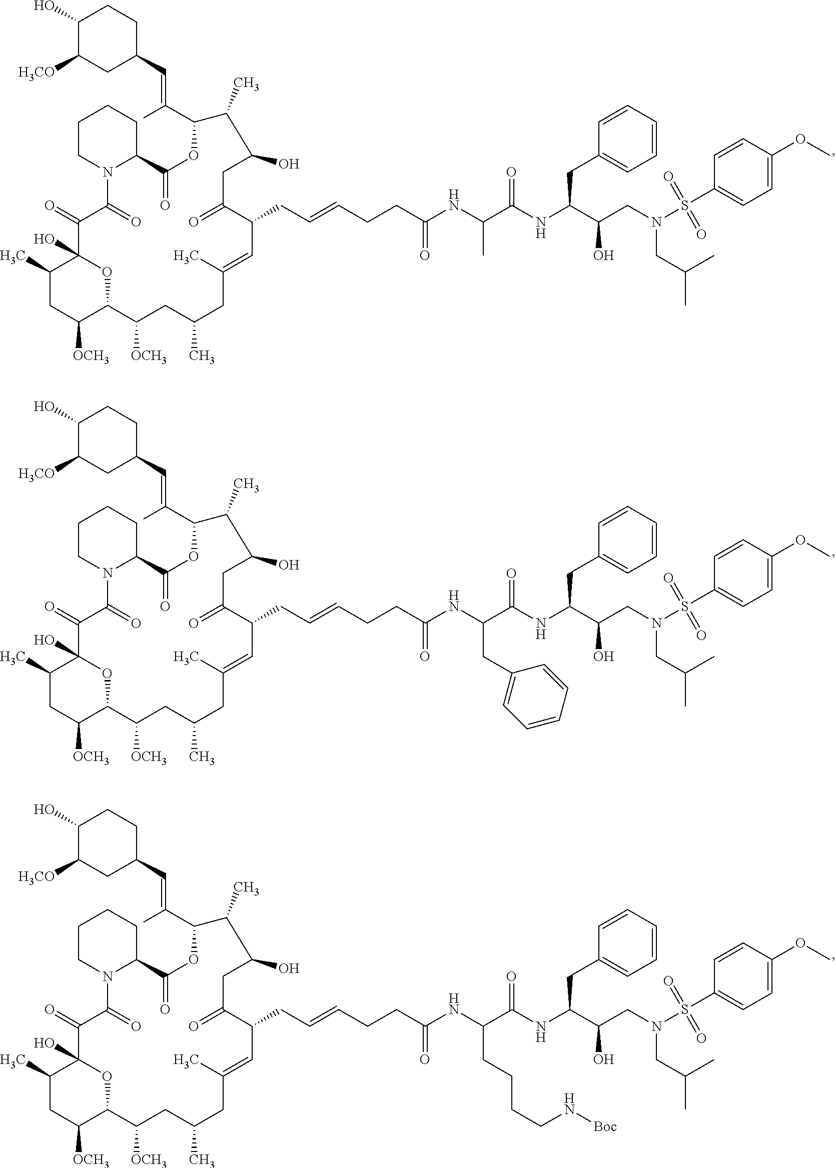

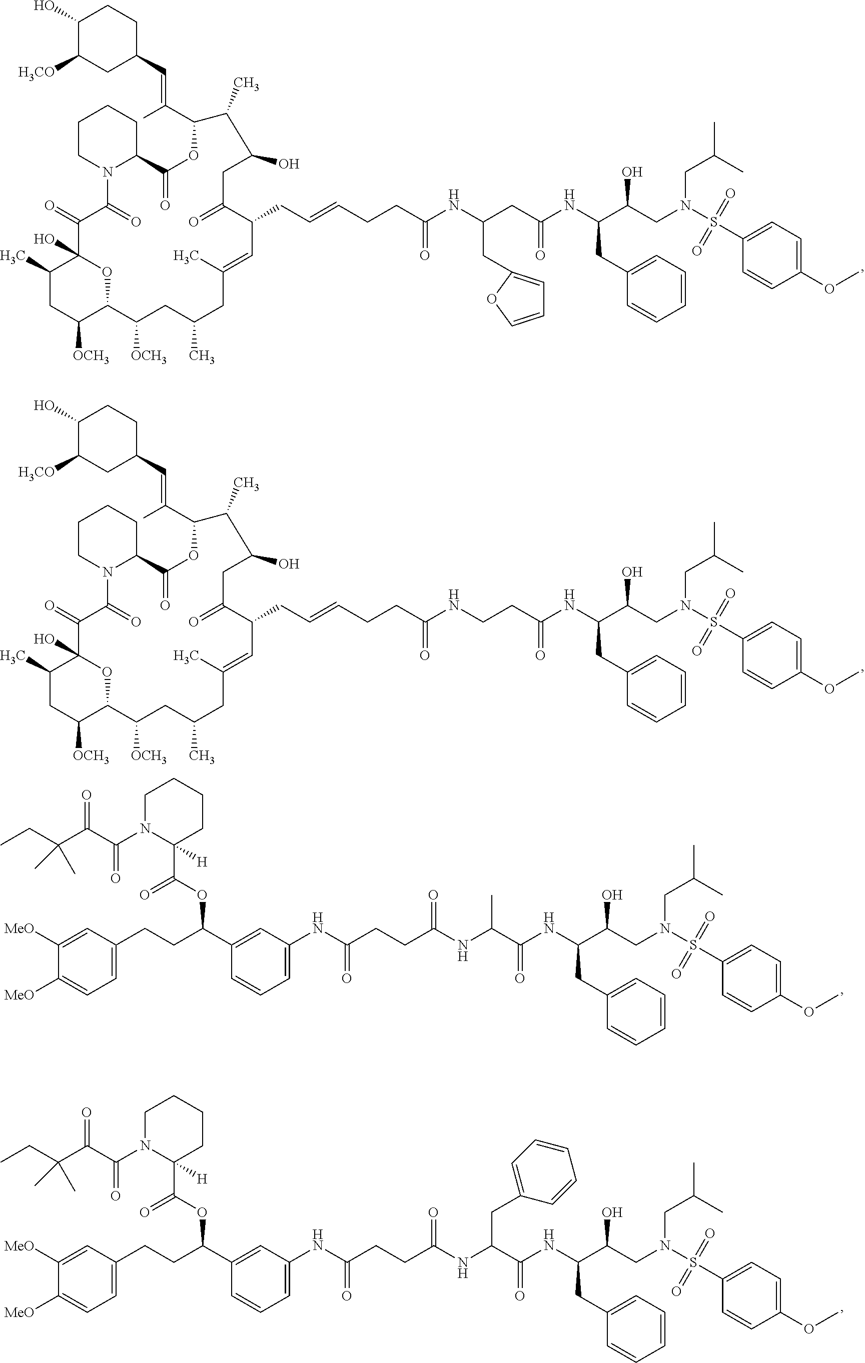

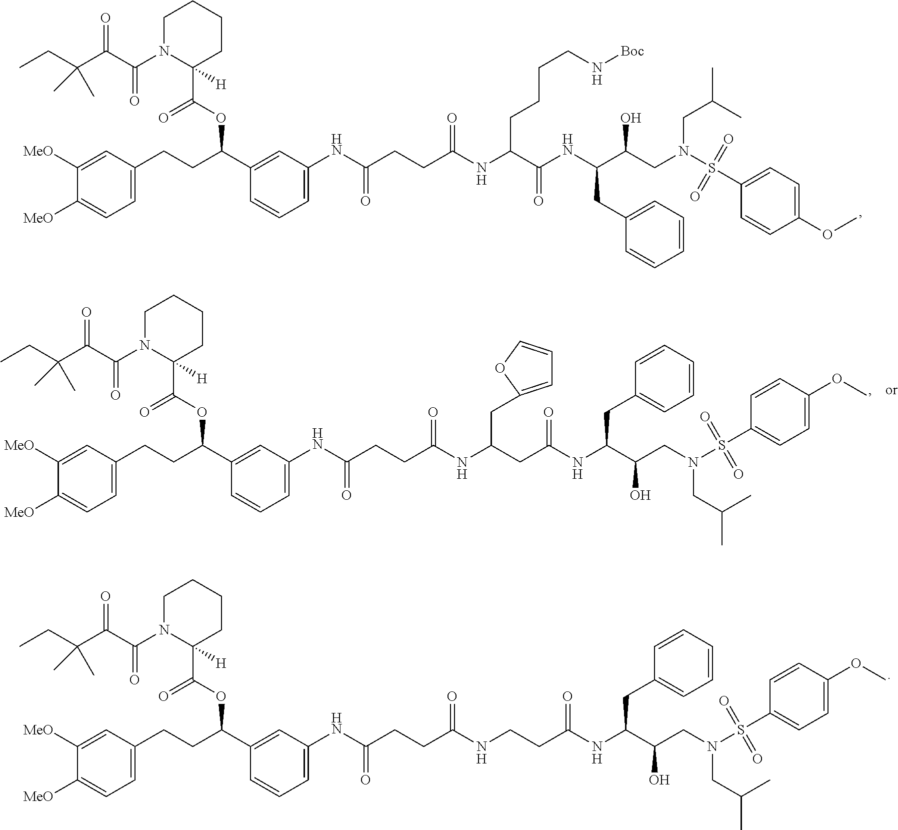

2. The compound of claim 1, wherein R¹ is not a monovalent human immunodeficiency (HIV) protease inhibitor or an amyloid β aggregation inhibitor.

3. The compound of claim 1, wherein the immunophilin-binding moiety is a cyclophilin-binding moiety or an FKBP-binding moiety.

4. The compound of claim 1, wherein the immunophilin-binding moiety is

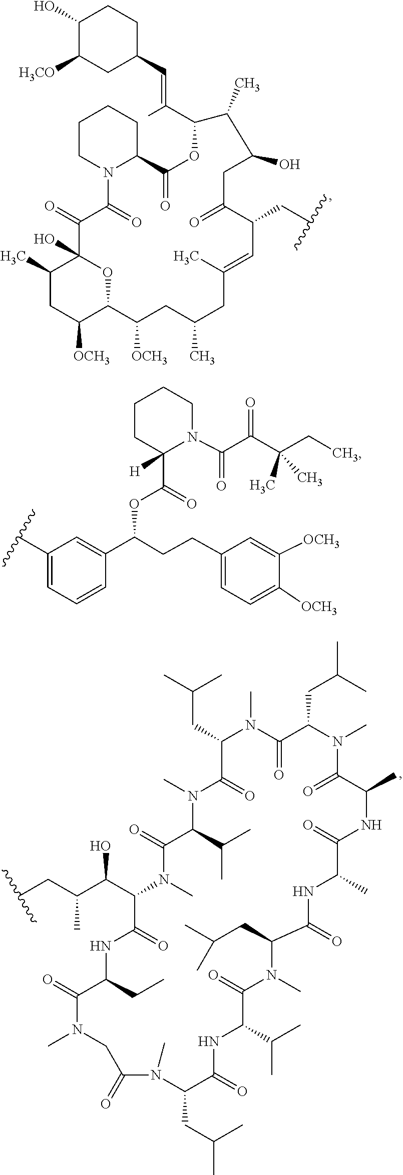

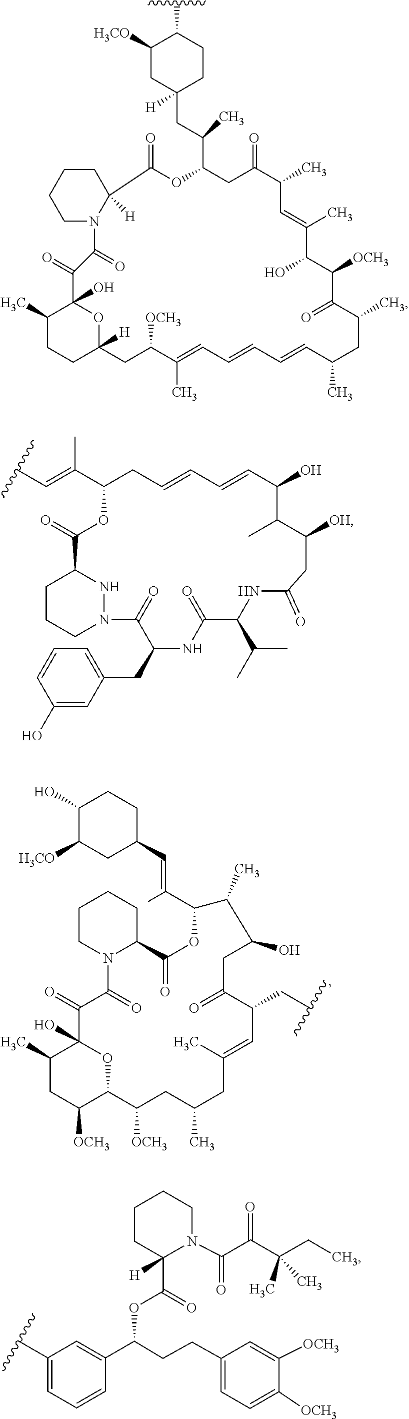

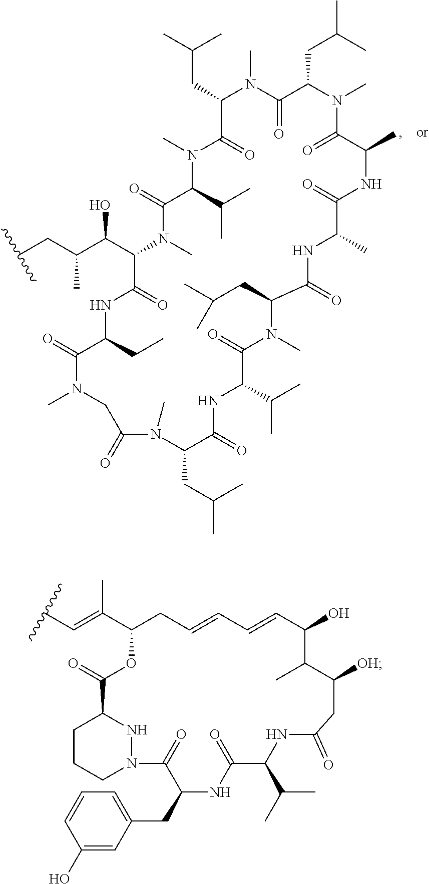 [SMILES: *C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; */C=C(\C)[C@@H]1C/C=C/C=C/[C@@H](O)C(C)[C@@H](O)CC(=O)N[C@@H](C(C)C)C(=O)N[C@@H](Cc2cccc(O)c2)C(=O)N2CCC[C@H](N2)C(=O)O1]

5. (canceled)

6. The compound of claim 1, wherein L¹ is L²-L³-L⁴-L⁵-L⁶;
L² is connected directly to the moiety of an immunophilin-binding compound;
L² is a bond, —S(O)₂—, —N(R²)—, —O—, —S—, —C(O)—, —C(O)N(R²)—, —N(R²)C(O)—, —N(R²)C(O)NH—, —NHC(O)N(R²)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —S(O)₂—, —N(R³)—, —O—, —S—, —C(O)—, —C(O)N(R³)—, —N(R³)C(O)—, —N(R³)C(O)NH—, —NHC(O)N(R³)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is a bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁵ is a bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L⁶ is a bond, —S(O)₂—, —N(R⁶)—, —O—, —S—, —C(O)—, —C(O)N(R⁶)—, —N(R⁶)C(O)—, —N(R⁶)C(O)NH—, —NHC(O)N(R⁶)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R², R³, R⁴, R⁵, and R⁶ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

7-9. (canceled)

10. The compound of claim 1, wherein L¹ is a bond, a substituted or unsubstituted alkylene or substituted or unsubstituted heteroalkylene, an unsubstituted C₃-C₇ alkylene, an oxo-substituted C₃-C₇ alkylene, an unsubstituted 3 to 17 membered heteroalkylene, or an oxo-substituted 3 to 17 membered heteroalkylene.

11. The compound of claim 1, wherein L¹ is a bond,

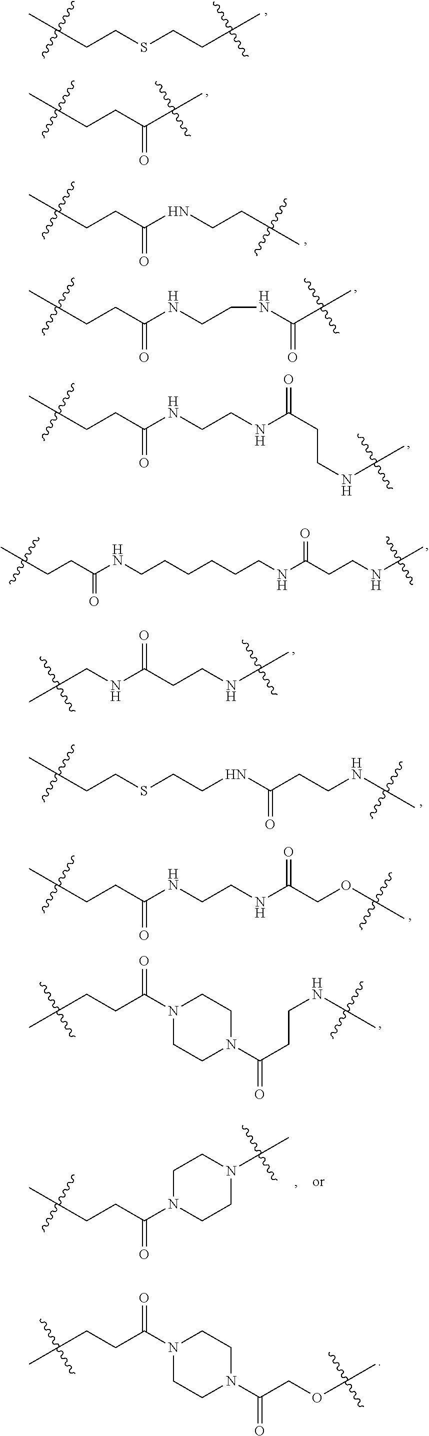

12-13. (canceled)

14. The compound of claim 1, wherein R¹ is a monovalent kinase inhibitor; and/or the kinase is not mTOR.

15. (canceled)

16. The compound of claim 14, wherein the monovalent kinase inhibitor is a monovalent Src kinase inhibitor, the monovalent Src kinase inhibitor is a monovalent dasatinib or monovalent dasatinib derivative, and/or the monovalent dasatinib derivative has the formula:

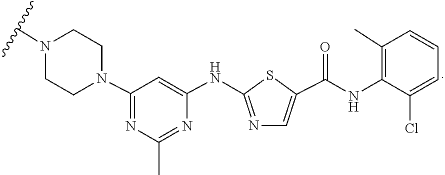 [SMILES: *N1CCN(c2cc(Nc3ncc(C(=O)Nc4c(C)cccc4Cl)s3)nc(C)n2)CC1]

17-18. (canceled)

19. The compound of claim 14, wherein the monovalent kinase inhibitor is a monovalent Raf inhibitor, VEGFR inhibitor, PDGFR inhibitor, or c-Kit inhibitor, the monovalent Raf inhibitor, VEGFR inhibitor, PDGFR inhibitor, or c-Kit inhibitor is a monovalent sorafenib or monovalent sorafenib derivative, and/or the monovalent sorafenib derivative has the formula:

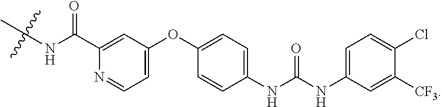 [SMILES: *NC(=O)c1cc(Oc2ccc(NC(=O)Nc3ccc(Cl)c(C(F)(F)F)c3)cc2)ccn1]

20-21. (canceled)

22. The compound of claim 14, wherein the monovalent kinase inhibitor is a monovalent EGFR inhibitor, the monovalent EGFR inhibitor is a monovalent lapatinib, monovalent lapatinib derivative, monovalent erlotinib, monovalent erlotinib derivative, monovalent gefitinib, or monovalent gefitinib derivative, and/or the monovalent EGFR inhibitor has the formula:

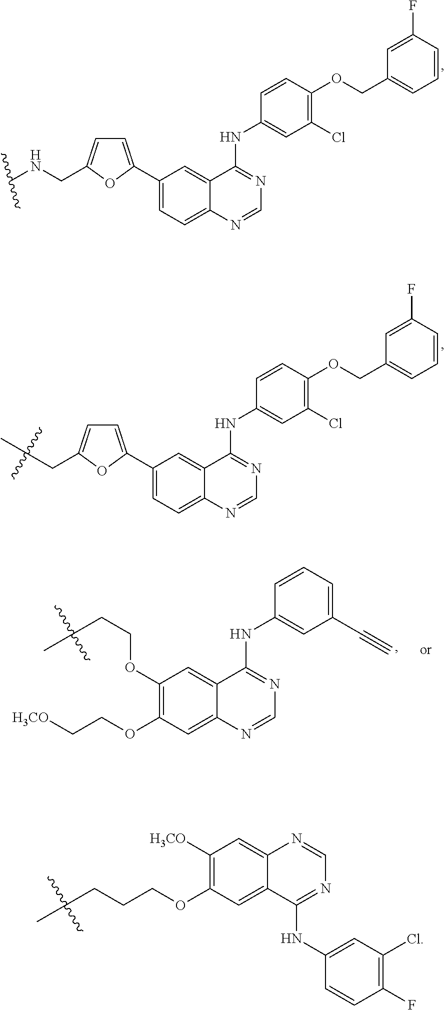 [SMILES: *NCc1ccc(-c2ccc3ncnc(Nc4ccc(OCc5cccc(F)c5)c(Cl)c4)c3c2)o1 ; *Cc1ccc(-c2ccc3ncnc(Nc4ccc(OCc5cccc(F)c5)c(Cl)c4)c3c2)o1 ; *CCOc1cc2c(Nc3cccc(C#C)c3)ncnc2cc1OCCOC ; *CCCOc1cc2c(Nc3ccc(F)c(Cl)c3)ncnc2cc1OC]

23-24. (canceled)

25. The compound of claim 14, wherein the monovalent kinase inhibitor is a monovalent LRRK2 inhibitor, the monovalent LRRK2 inhibitor is a monovalent GNE-7915 or monovalent GNE-7915 derivative, and/or the monovalent GNE-7915 derivative has the formula:

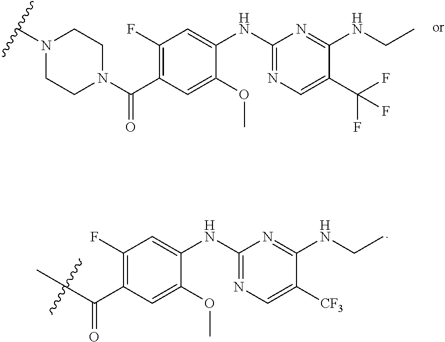 [SMILES: *N1CCN(C(=O)c2cc(OC)c(Nc3ncc(C(F)(F)F)c(NCC)n3)cc2F)CC1 ; *C(=O)c1cc(OC)c(Nc2ncc(C(F)(F)F)c(NCC)n2)cc1F]

26-27. (canceled)

28. The compound of claim 14, wherein the monovalent kinase inhibitor is a monovalent MAP4K inhibitor, the monovalent MAP4K inhibitor is a monovalent HGK inhibitor and/or the monovalent HGK inhibitor has the formula:

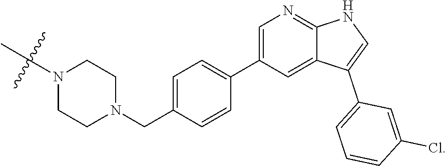 [SMILES: *N1CCN(Cc2ccc(-c3cnc4[nH]cc(-c5cccc(Cl)c5)c4c3)cc2)CC1]

29-30. (canceled)

31. The compound of claim 14, wherein the monovalent kinase inhibitor is a monovalent MAP3K inhibitor, the monovalent MAP3K inhibitor is a monovalent DLK inhibitor, and/or the monovalent DLK inhibitor has the formula:

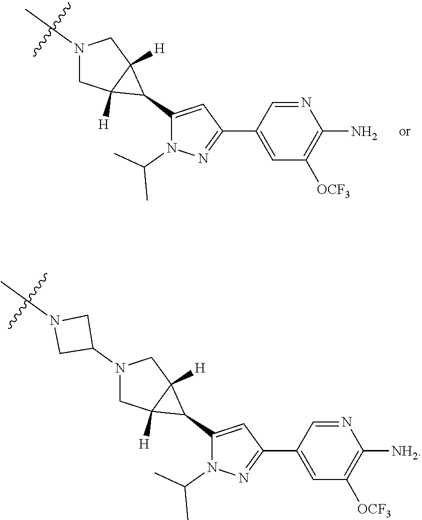 [SMILES: *N1C[C@@H]2[C@H](C1)[C@H]2c1cc(-c2cnc(N)c(OC(F)(F)F)c2)nn1C(C)C ; *N1CC(N2C[C@@H]3[C@H](C2)[C@H]3c2cc(-c3cnc(N)c(OC(F)(F)F)c3)nn2C(C)C)C1]

32-33. (canceled)

34. The compound of claim 1, wherein R¹ is a monovalent KRAS inhibitor.

35. The compound of claim 34, wherein the monovalent KRAS inhibitor is a monovalent KRAS G12C inhibitor or a monovalent KRAS M72C inhibitor, and the monovalent KRAS inhibitor has the formula:

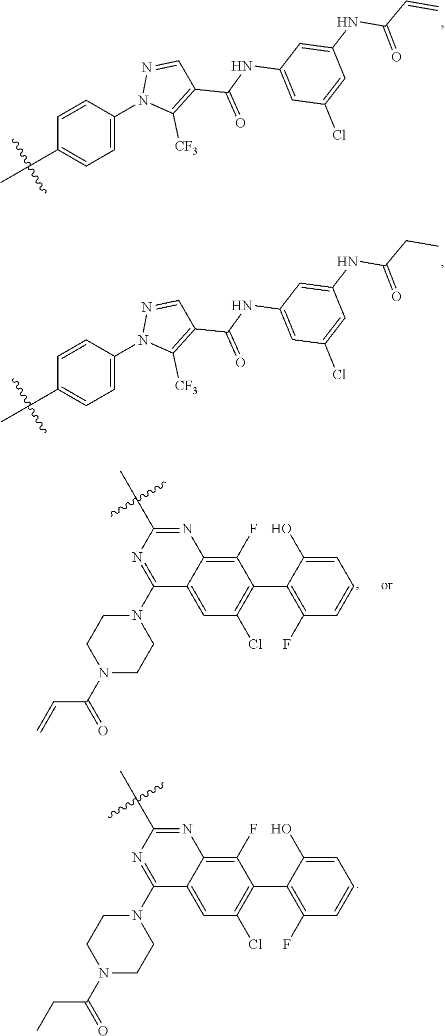

36. (canceled)

37. The compound of claim 1, wherein the compound is not a calcineurin inhibitor.

38. A pharmaceutical composition comprising a pharmaceutically acceptable excipient and a compound of one of claim 1.

39. A method of treating a disease associated with aberrant enzyme activity in a subject in need of such treatment, comprising administering a compound of claim 1 to the subject.

40. The method of claim 39, wherein the enzyme activity is a kinase activity, and the kinase activity is in the CNS of the subject.

1. (canceled)

42. A method of treating a disease in a subject in need of such treatment, comprising administering a compound of claim 1 to the subject, wherein the disease is a viral disease, cancer, or a neurodegenerative disease.

43. (canceled)

44. The method of claim 42, wherein the cancer is glioblastoma or glioma, and the neurodegenerative is Parkinson's Disease, Amyotrophic lateral sclerosis (ALS), or Alzheimer's disease.

45-48. (canceled)
